(19) United States
(12) Patent Application Publication
(10) Pub. No.: US 2002/0192681 A1
(43) Pub. Date: Dec. 19, 2002

(54) Response of dendritic cells to a diverse set of pathogens

(75) Inventors: Nir Hacohen, Cambridge, MA (US); Qian Huang, Arlington, MA (US); Dongyu Liu, Malden, MA (US)

Correspondence Address: Lisa M. Treannie, Esq., HAMILTON, BROOK, SMITH & REYNOLDS, P.C., 530 Virginia Road, P.O. Box 9133, Concord, MA, 01742-9133, US

(73) Assignee: Whitehead Institute for Biomedical Research, Cambridge, MA

(21) Appl. No.: 10/085,982

(22) Filed: Oct. 24, 2001

Related U.S. Application Data
(60) Provisional application No. 60/242,800, filed on Oct. 24, 2000.

(51) Int. Cl. C12Q 1/68; G06F 19/00; G01N 33/48; G01N 33/50

(52) U.S. Cl. 435/6; 702/20

(57) ABSTRACT

Distinct gene expression programs activated in response to different pathogens in dendritic cells are disclosed. Methods of diagnosis and treatment are also disclosed.

### RELATED APPLICATIONS

This application claims the benefit of U.S. Provisional Application No. 60/242,800, filed Oct. 24, 2000, the entire teachings of which are incorporated herein by reference.

### BACKGROUND OF THE INVENTION

The human immune system is continually challenged by a diverse array of pathogens. The immune system first reacts to a pathogen with the innate immune response via cytokines and chemokines that modulate neutrophils, natural killer cells and other cell types, followed later by the adaptive immune response. It has been hypothesized that the innate immune system is equipped with receptors capable of recognizing many of the invading infectious agents and able to react to optimize the immune response.

The initiation of the immune response and the adaptive response is critically dependent on the activation of a subset of the antigen presenting cells termed “dendritic cells”. Dendritic cells reside in most tissues, mainly in an immature stage, where they survey for incoming pathogens or antigens. Once they capture and process antigens, they undergo maturation and activation, which dramatically increases their ability to activate T cells. Upon activation, dendritic cells transport antigens to lymphoid organs where they can trigger a specific T cell adaptive immune response. The capacity of dendritic cells to recognize invading pathogens and become activated is the first critical event in the initiation of the immune response, such as activation of naive CD4+ and CD8+ cells which in turn can kill infected cells or tumor cells. In contrast, dendritic cells are also involved in maintaining tolerance to antigens by deleting autoreactive lymphocytes, thereby reducing autoimmune responses. Although dendritic cells are essential in immunity, little is known about how dendritic cells control the innate and adaptive immune response to such an array of pathogens.

### SUMMARY OF THE INVENTION

The present invention provides a method for identifying a pathogen by the pattern of gene expression induced by dendritic cells in response the pathogen. As described herein, distinct gene expression programs are activated in response to different pathogens. The present invention is useful in the diagnosis, prognosis and treatment of disease.

In one embodiment, the present invention is directed toward a method of identifying infection by a pathogen comprising the steps of isolating mRNA from one or more dendritic cells; and determining gene expression of at least one stimulus-specific gene, wherein expression of a stimulus-specific gene is indicative of infection by a pathogen to which the stimulus-specific gene is specific. In an alternate embodiment, the present invention is directed toward a method of identifying a pathogen comprising the steps of contacting one or more immature dendritic cells with a pathogen or with one or more immunogenic components thereof; isolating and labeling mRNA from said dendritic cells; detecting labeled mRNA from said dendritic cells such that a gene expression profile is produced; and analyzing the gene expression profile relative to one or more reference gene expression profiles such that at least one stimulus-specific gene is identified, thereby identifying a pathogen for which the stimulus is specific.

In another embodiment, the present invention is directed toward a method of diagnosing infection in a mammal comprising the steps of isolating mRNA from one or more dendritic cells from said mammal; contacting said mRNA with at least one stimulus-responsive gene probe wherein hybridization of a stimulus-responsive gene probe to said mRNA is indicative of infection in said mammal. In a particular embodiment, the stimulus-responsive gene is stimulus-specific. In another particular embodiment, the stimulus-responsive gene is a common stimulus-responsive gene.

In another embodiment, the present invention is directed toward a method of diagnosing infection in a mammal comprising the steps of isolating proteins from one or more dendritic cells from said mammal; contacting said proteins with at least one antibody to a protein encoded by a stimulus-specific gene, wherein binding of said antibody to said protein is indicative of infection in said mammal.

In another embodiment, the present invention is directed toward a method of diagnosing infection by a pathogen in a mammal comprising the steps of isolating mRNA from one or more dendritic cells in a mammal; and determining gene expression of at least one stimulus-specific gene, wherein expression of a stimulus-specific gene is indicative of infection by pathogen to which the stimulus-specific gene is specific.

In a particular embodiment, stimulus-responsive gene expression is decreased. In another particular embodiment, stimulus-responsive gene expression is increased.

In another embodiment, the present invention is directed toward a method of predicting prognosis for an infected individual by analyzing gene profiles of stimulus-responsive genes, wherein a gene profile is correlated with a clinical outcome.

In another embodiment, the present invention is directed to a method of formulating a therapeutic regimen comprising the steps of identifying the pathogen, and formulating a therapeutic regimen accordingly. Additionally, repeated assessment of a patient for a pathogen can determine the effectiveness of a therapeutic regimen which can then be altered accordingly.

In another embodiment, the present invention is directed toward a method of optimizing a vaccine comprising the steps of contacting one or more immature dendritic cells with test vaccines; isolating mRNA from said dendritic cells; determining gene profiles in said dendritic cells; and selecting a test vaccine which elicits the gene expression profile which is indicative of an optimized vaccine.

In another embodiment, the present invention is directed toward an ex vivo therapeutic treatment for a pathogen comprising the steps of contacting a patient's dendritic cells with a pathogen or components thereof such that said dendritic cells become activated; and returning the activated dendritic cells to the patient such that activated dendritic cells trigger an immune response against said pathogen.

In another embodiment, the present invention is directed toward an ex vivo therapeutic treatment for a tumor comprising the steps of contacting a patient's dendritic cells with tumor cells or components thereof such that said dendritic cells become activated; and returning the activated dendritic cells to the patient such that activated dendritic cells trigger an immune response against said tumor cells or components thereof.

In another embodiment, the present invention is directed toward an ex vivo therapeutic treatment for autoimmunity comprising the steps of contacting a patient's dendritic cells with self-antigens or components thereof such that said dendritic cells become activated; and returning the activated dendritic cells to the patient such that activated dendritic cells do not trigger an immune response against said self-antigens or components thereof.

In another embodiment, the present invention is directed toward an ex vivo therapeutic treatment for graft-rejection comprising the steps of contacting a patient's dendritic cells with graft-tissue or components thereof such that said dendritic cells become activated; returning the activated dendritic cells to the patient such that activated dendritic cells do not trigger an immune response against said graft-tissue or components thereof.

In another embodiment, the present invention is directed to a method of measuring the immune response to a stimulus comprising the steps of contacting one or more dendritic cells with a stimulus; isolating mRNA from said dendritic cells and determining a gene profile such that at least one stimulus-responsive gene is identified which is indicative of an immune response.

In another embodiment, the present invention is directed to a method of measuring the immune response to a stimulus comprising the steps of contacting one or more dendritic cells with a stimulus; isolating and labeling mRNA from said dendritic cells; contacting a DNA microarray with labeled mRNA from said dendritic cells; and analyzing the gene profile relative to control stimulus such that at least one stimulus-responsive gene is identified which is indicative of an immune response. In a particular embodiment, the dendritic cells are obtained from peripheral blood. In another particular embodiment, the stimulus is selected from the group consisting of bacteria, fungi, viruses, or components thereof. In another particular embodiment, the stimulus is selected from the group consisting of Escherichia coli, Staphylococcus aurens, influenza virus, Candida albicans, lipopolysaccharide (LPS), polyI:C, and yeast mannan. In a particular embodiment, the stimulus is selected from the group consisting of physical, chemical, or electrical stimulus. In another particular embodiment, the stimulus is selected from the group consisting of inorganic chemicals and organic chemicals. In another embodiment, the stimulus comprises a combination selected from the group consisting of: inorganic chemicals and organic chemicals. In a particular embodiment, the DNA microarray is Affymetrix GeneChip® HU 6800. In another particular embodiment, the DNA microarray is Affymetrix GeneChip® Human Genome U95 set.

In another embodiment, the stimulus-responsive gene's expression is increased in response to the stimulus. In another embodiment, the stimulus-responsive gene's expression is decreased in response to the stimulus. In another embodiment, the stimulus-responsive gene is stimulus-specific.

In another embodiment the present invention is directed toward a method of measuring the gene profile in dendritic cells in response to a stimulus comprising the steps of contacting immature dendritic cells with a stimulus, isolating and labeling mRNA from said dendritic cells and measuring and analyzing the gene profile relative to control stimulus such that at least one stimulus-responsive gene is identified.

In another embodiment the present invention is directed toward the production of databases containing stimulus-responsive genes comprising the steps of contacting dendritic cells with a stimulus; isolating and labeling mRNA from said dendritic cells; contacting a DNA microarray with labeled mRNA from said dendritic cells; and measuring and analyzing the gene profile relative to control stimulus such that a database containing at least one stimulus-responsive gene is generated. In another embodiment the present invention is directed toward the formation of databases containing stimulus-specific genes comprising the steps of contacting dendritic cells with a stimulus; isolating and labeling mRNA from said dendritic cells; contacting a DNA microarray with labeled mRNA from said dendritic cells; and measuring and analyzing the gene profile relative to control stimulus such that a database containing at least one stimulus-specific gene is generated. In another embodiment the present invention is directed toward the formation of a database containing common-immune response genes comprising the steps of contacting dendritic cells with a stimulus; isolating and labeling mRNA from said dendritic cells; contacting a DNA microarray with labeled mRNA from said dendritic cells; and measuring and analyzing the gene profile relative to control stimulus such that a database containing at least one common stimulus-responsive gene is generated. In another embodiment, the invention relates to databases of stimulus-responsive genes, databases of stimulus-specific genes and databases of common-immune response genes.

In another embodiment, the invention is directed to a method of identifying a pathogen comprising the steps of contacting one or more immature dendritic cells with a stimulus; isolating mRNA from said dendritic cells; and determining a gene profile such that at least one stimulus-specific gene is identified thereby identifying a pathogen for which the stimulus-specific gene is specific. In another embodiment, the invention is directed to a method of identifying a pathogen comprising the steps of contacting one or more immature dendritic cells with a stimulus; isolating and labeling mRNA from said dendritic cells; contacting a DNA microarray with labeled mRNA from said dendritic cells; and measuring and analyzing the gene profile relative to control stimulus such that at least one stimulus-specific gene is identified thereby identifying a pathogen for which the stimulus-specific gene is specific.

In another embodiment, the invention is directed a method of diagnosing infection by a pathogen comprising the steps of: isolating mRNA from dendritic cells; and determining a gene profile such that at least one stimulus-specific gene is identified thereby identifying a pathogen for which the stimulus-specific gene is specific which is indicative of infection. In another embodiment, the invention is directed a method of diagnosing infection by a pathogen comprising the steps of isolating and labeling mRNA from dendritic cells; contacting a DNA microarray with labeled mRNA from said dendritic cells; and measuring and analyzing the gene profile relative to control stimulus such that at least one stimulus-specific gene is identified thereby identifying a pathogen for which the stimulus-specific gene is specific which is indicative of infection.

The identification of stimulus-specific, stimulus-responsive and common stimulus-responsive genes in dendritic cells provides new therapeutic, diagnostic and prognostic tools of importance in medicine and research.

### BRIEF DESCRIPTION OF THE DRAWINGS

FIGS. 1A-1GG list dendritic cell genes induced upon stimulation with E. coli. Column one cites the Affymetrix identification number (from the Affymetrix GeneChip® used as described herein). Column two cites the Genbank or TIGR accession number. Column three cites the gene abbreviation. Column four cites the gene name. Columns five through seven cite the gene score for stimulation with E. coli (one donor per column). Columns eight through ten cite the gene score for stimulation with influenza (one donor per column). Columns eleven through thirteen cite the gene score for stimulation with Candida albicans (one donor per column).

FIGS. 2A-2Z list dendritic cell genes induced upon stimulation with influenza virus. Column one cites the Affymetrix identification number (from the Affymetrix GeneChip® used as described herein). Column two cites the Genbank or TIGR accession number. Column three cites the gene abbreviation. Column four cites the gene name. Columns five through seven cite the gene score for stimulation with E. coli (one donor per column). Columns eight through ten cite the gene score for stimulation with influenza (one donor per column). Columns eleven through thirteen cite the gene score for stimulation with Candida albicans (one donor per column).

FIGS. 3A-3N list dendritic cell genes induced upon stimulation with Candida albicans. Column one cites the Affymetrix identification number (from the Affymetrix GeneChip® used as described herein). Column two cites the Genbank or TIGR accession number. Column three cites the gene abbreviation. Column four cites the gene name. Columns five through seven cite the gene score for stimulation with E. coli (one donor per column). Columns eight through ten cite the gene score for stimulation with influenza (one donor per column). Columns eleven through thirteen cite the gene score for stimulation with Candida albicans (one donor per column).

FIGS. 4A-4F list dendritic cell genes specifically induced by stimulation with E. coli. Column one cites the Affymetrix identification number (from the Affymetrix GeneChip® used as described herein). Column two cites the Genbank or TIGR accession number. Column three cites the gene abbreviation. Column four cites the gene name. Columns five through seven cite the gene score for stimulation with E. coli (one donor per column). Columns eight through ten cite the gene score for stimulation with influenza (one donor per column). Columns eleven through thirteen cite the gene score for stimulation with Candida albicans (one donor per column).

FIGS. 5A-5C list dendritic cell genes specifically induced by stimulation with influenza virus. Column one cites the Affymetrix identification number (from the Affymetrix GeneChip® used as described herein). Column two cites the Genbank or TIGR accession number. Column three cites the gene abbreviation. Column four cites the gene name. Columns five through seven cite the gene score for stimulation with E. coli (one donor per column). Columns eight through ten cite the gene score for stimulation with influenza (one donor per column). Columns eleven through thirteen cite the gene score for stimulation with Candida albicans (one donor per column).

FIGS. 6A-6I list dendritic cell genes induced by E. coli, influenza virus, and Candida albicans. Column one cites the Affymetrix identification number (from the Affymetrix GeneChip® used as described herein). Column two cites the Genbank or TIGR accession number. Column three cites the gene abbreviation. Column four cites the gene name. Columns five through seven cite the gene score for stimulation with E. coli (one donor per column). Columns eight through ten cite the gene score for stimulation with influenza (one donor per column). Columns eleven through thirteen cite the gene score for stimulation with Candida albicans (one donor per column).

FIG. 7A is a diagram which depicts the number of dendritic cell genes regulated for each stimulus and the number of genes which overlap in response to various stimuli.

FIG. 7B is an image of a gene profile and a diagram which represents the dendritic cell genes regulated in response to E. coli and C. albicans over time.

FIG. 7C is an image of a gene profile and a diagram which represents the dendritic cell genes regulated in response to E. coli and influenza over time.

FIG. 7D is an image of a gene profile of common response dendritic cell genes stimulated by E. coli, C. albicans and influenza.

FIG. 7E is an image of a gene profile of differential response dendritic cell genes stimulated by E. coli, C. albicans and influenza.

FIG. 8A is a schematic diagram of expression kinetics of common response genes in dendritic cells over time. Genes were grouped by the temporal nature of their expression and duration of their expression (i.e. early transient, ET; early sustained, ES; middle transient, MT; middle sustained, MS; late transient, LT; and late sustained, LS).

FIG. 8B is a list of dendritic cell genes grouped by their temporal expression kinetics and gene type.

FIG. 8C is a diagram dendritic cell life cycle stages. The relative timing of dendritic cell maturation which include phagocytosis of pathogens, activation of the innate immune response, migration to the lymph node, antigen presentation and stimulation of the adaptive immune cells, and apoptosis are shown.

FIG. 9A is a diagram of the number of dendritic cell genes regulated in response to E. coli, C. albicans, or influenza or their component molecule LPS, mannan or dsRNA, respectively.

FIG. 9B is a diagram of the number of dendritic cell genes regulated in response to E. coli or the mannan or dsRNA.

FIGS. 10A-C are a list of the functional categories of dendritic cells genes differentially regulated in response to E. coli, C. albicans, and influenza.

FIGS. 11A-11AAAAAAA list dendritic cell genes induced upon stimulation with E. coli using the GeneChip® human genome U95 set. Column one cites the Affymetrix identification number (from the Affymetrix GeneChips® used as described herein). Column two cites the gene name. Column three cites the gene abbreviation. Column four cites the Genbank or TIGR accession number. Column five cites the gene score for stimulation with E. coli. Column six cites the gene score for stimulation with Candida albicans. Column seven cites the gene score for stimulation with influenza.

FIGS. 12A-12AA list dendritic cell genes specifically induced by stimulation with E. coli using the GeneChip® human genome U95 set. Column one cites the Affymetrix identification number (from the Affymetrix GeneChip® used as described herein). Column two cites the gene name. Column three cites the gene abbreviation. Column four cites the Genbank or TIGR accession number. Column five cites the gene score for stimulation with E. coli. Column six cites the gene score for stimulation with Candida albicans. Column seven cites the gene score for stimulation with influenza.

FIGS. 13A-13ZZZZZZ list dendritic cell genes induced upon stimulation with influenza virus using the GeneChip® human genome U95 set. Column one cites the Affymetrix identification number (from the Affymetrix GeneChip® used as described herein). Column two cites the gene name. Column three cites the gene abbreviation. Column four cites the Genbank or TIGR accession number. Column five cites the gene score for stimulation with E. coli. Column six cites the gene score for stimulation with Candida albicans. Column seven cites the gene score for stimulation with influenza.

FIGS. 14A-14BB list dendritic genes specifically induced by stimulation with influenza virus. Column one cites the Affymetrix identification number (from the Affymetrix GeneChip® used as described herein). Column two cites the gene name. Column three cites the gene abbreviation. Column four cites the Genbank or TIGR accession number. Column five cites the gene score for stimulation with E. coli. Column six cites the gene score for stimulation with Candida albicans. Column seven cites the gene score for stimulation with influenza.

FIGS. 15A-15KKKK list dendritic cell genes induced upon stimulation with Candida albicans using the GeneChip® human genome U95 set. Column one cites the Affymetrix identification number (from the Affymetrix GeneChip® used as described herein). Column two cites the gene name. Column three cites the gene abbreviation. Column four cites the Genbank or TIGR accession number. Column five cites the gene score for stimulation with E. coli. Column six cites the gene score for stimulation with Candida albicans. Column seven cites the gene score for stimulation with influenza.

FIGS. 16A-16F list dendritic cell genes specifically induced upon stimulation with Candida albicans using the GeneChip® human genome U95 set. Column one cites the Affymetrix identification number (from the Affymetrix GeneChip® used as described herein). Column two cites the gene name. Column three cites the gene abbreviation. Column four cites the Genbank or TIGR accession number. Column five cites the gene score for stimulation with E. coli. Column six cites the gene score for stimulation with Candida albicans. Column seven cites the gene score for stimulation with influenza.

FIGS. 17A-17II list dendritic genes induced by E. coli, influenza virus, and Candida albicans. Column one cites the Affymetrix identification number (from the Affymetrix GeneChip® used as described herein). Column two cites the gene name. Column three cites the gene abbreviation. Column four cites the Genbank or TIGR accession number. Column five cites the gene score for stimulation with E. coli. Column six cites the gene score for stimulation with Candida albicans. Column seven cites the gene score for stimulation with influenza.

### DETAILED DESCRIPTION OF THE INVENTION

Although dendritic cells play a critical role in the immune response to a wide array of pathogens, little is known about the molecular components of this response. The present invention is based, at least in part, on the discovery that exposure of dendritic cells to a pathogen results in a pathogen-specific pattern of gene expression. Thus, the present invention can be used to identify infectious agents based on gene expression profiles described herein. The present invention also provides methods for determining pathogen-specific pattern of gene expression for additional pathogens or components thereof. The findings described herein determine the key component(s) in each pathogen that triggers an immune response, thus providing robust targets for immunotherapy. Therefore, the present invention provides a better understanding of host-pathogen interaction, aids in identification of known and novel pathogens, and improves the diagnosis and treatment of infectious diseases.

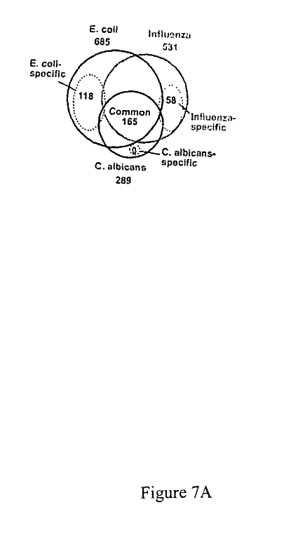

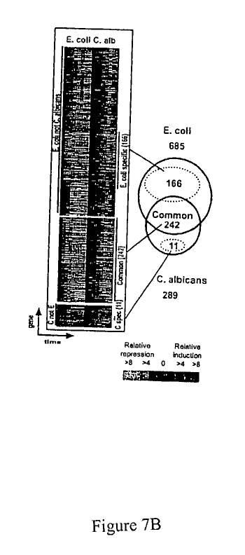

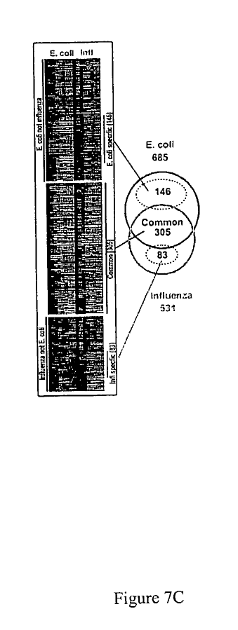

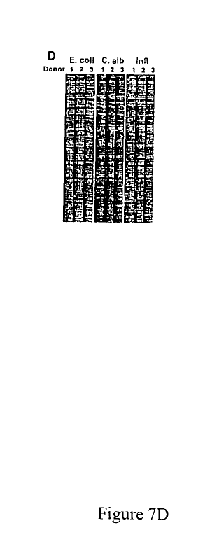

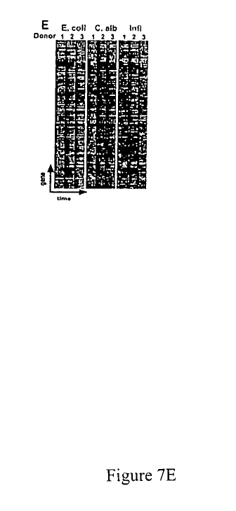

Identification of pathogens which cause disease is of paramount importance in providing effective treatment, as treatments may vary widely depending on the pathogen. In one embodiment, the present invention is a method of identifying a pathogen which has infected a vertebrate (e.g., a mammal) comprising the steps of isolating mRNA from one or more dendritic cells from the vertebrate; and determining a gene expression profile of at least one stimulus-specific gene, wherein the gene expression profile is indicative of infection by a pathogen to which the stimulus-specific gene(s) is specific. Methods of isolating RNA are described herein and well known to one of skill in the art. “Gene profile” or “gene expression profile” as used herein are defined as the level or amount of gene expression of particular genes as assessed by methods described herein or other methods known in the art. The gene expression profile can comprise data on one or more genes and can be measured at a single time point or over a period of time (see FIG. 7B). Pathogens can be identified by comparing the gene expression profile obtained from dendritic cells obtained from an infected vertebrate with one or more stimulus-specific gene expression profiles (e.g., in a database) such as those shown in FIG. 7E. The pathogen may be a family or class of pathogens which is identified by a particular gene profile. For example, the pathogen can be a member of the Eschericia family (e.g., E. coli), a member of the Candida family (e.g., Candida albicans) or a member of the influenza virus family. Therefore, the present invention can identify the class of pathogen causing a disease or illness and thus, aid in the selection of a treatment regime. A “treatment regime” as used herein refers to the clinical therapy a patient receives to ablate, alleviate or attenuate the disease or malady.

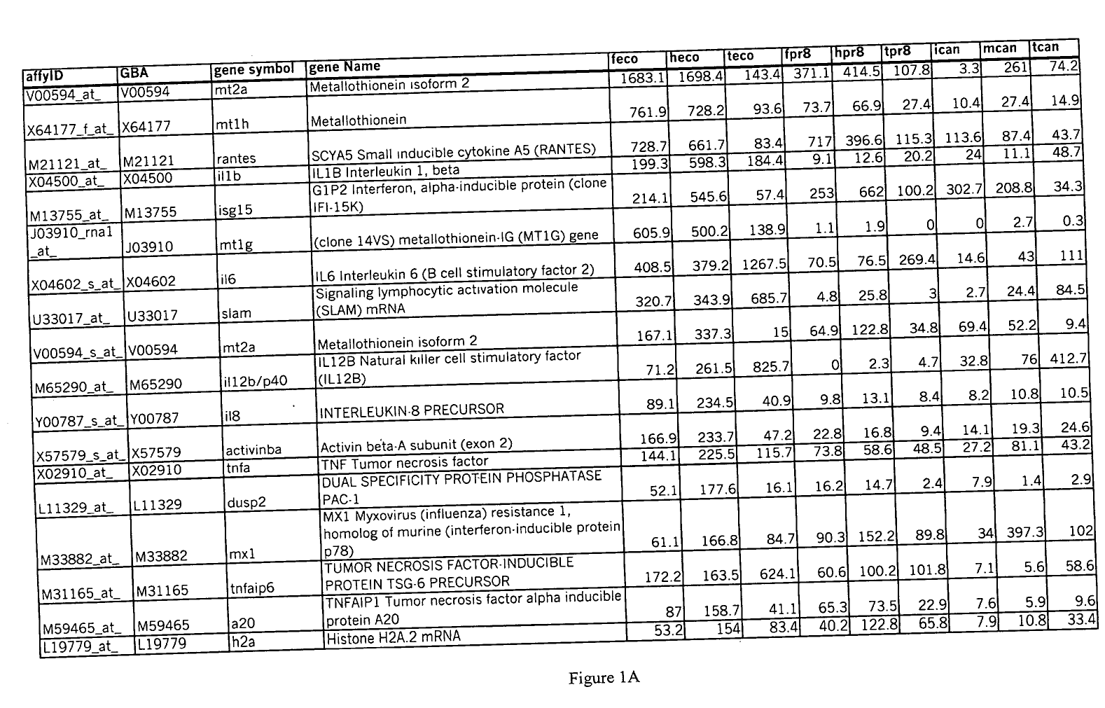

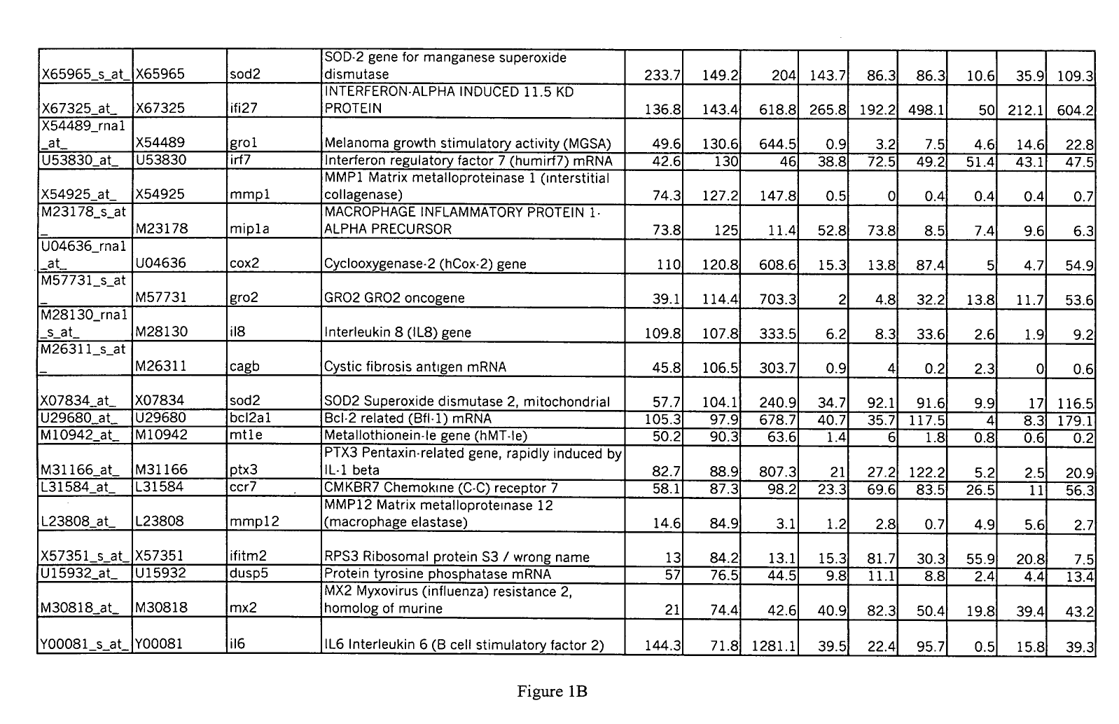

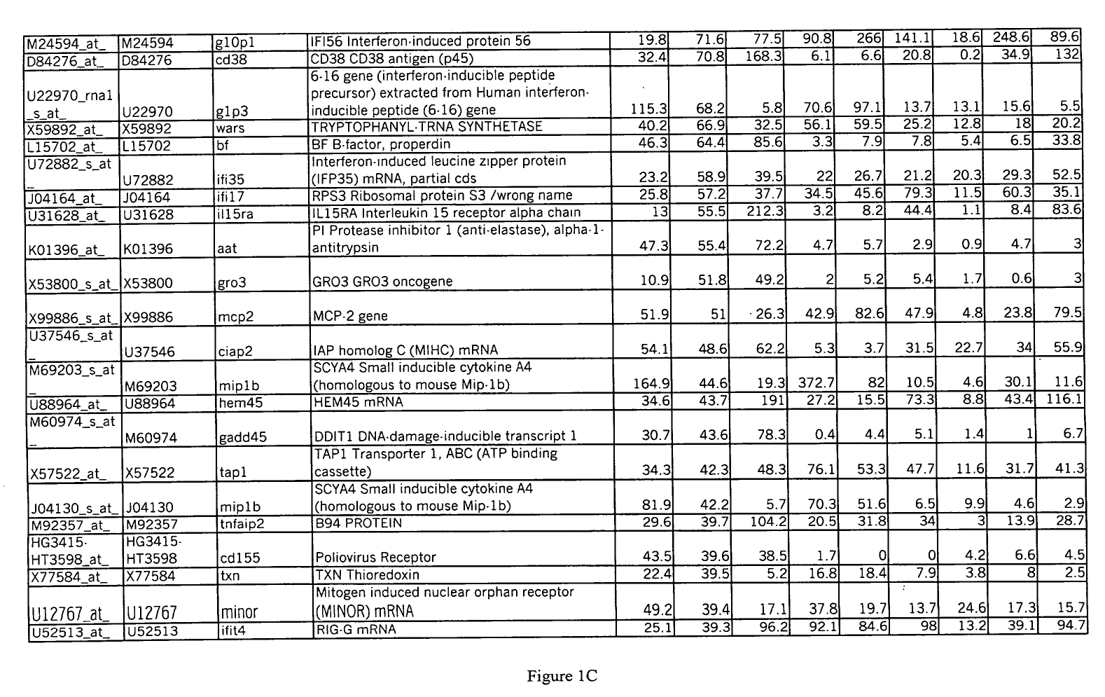

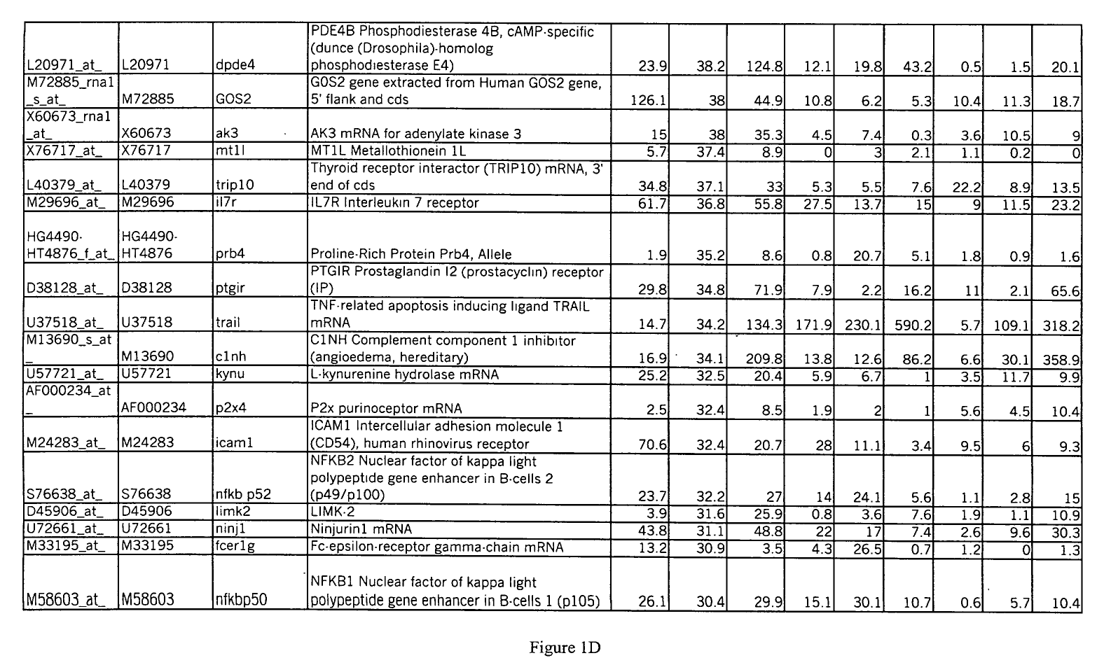

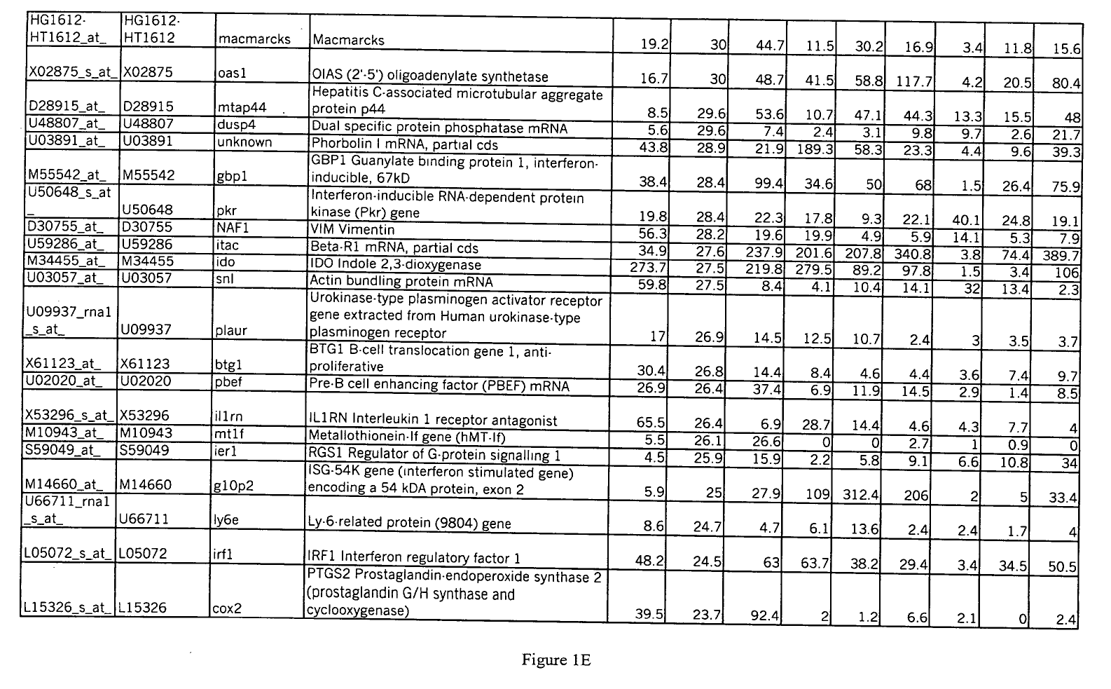

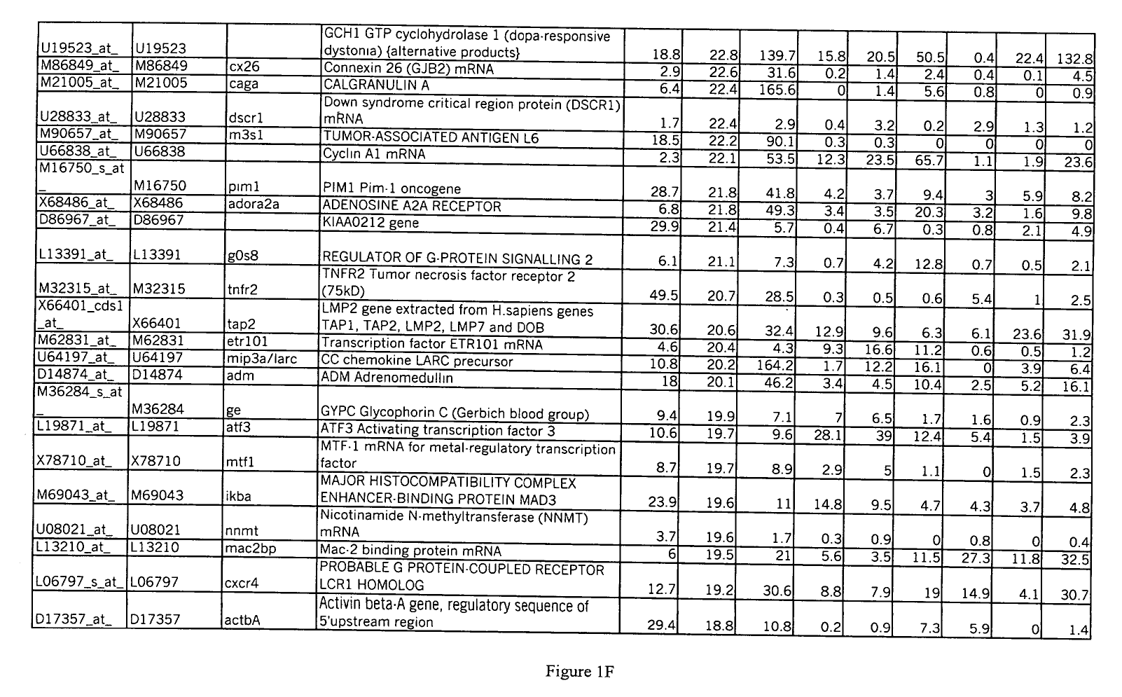

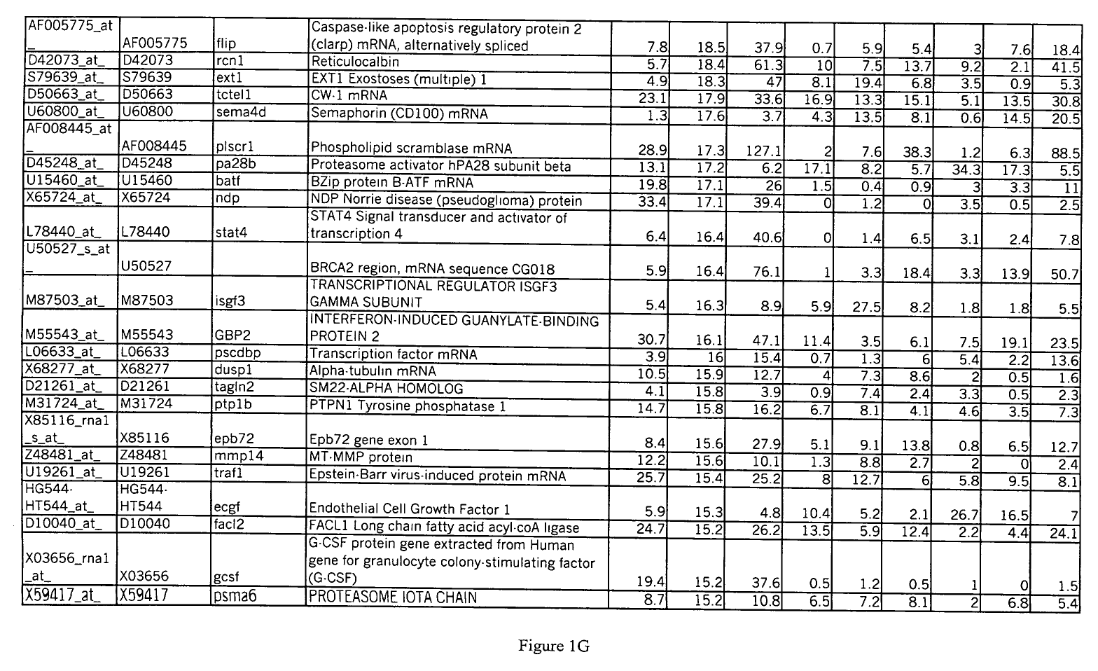

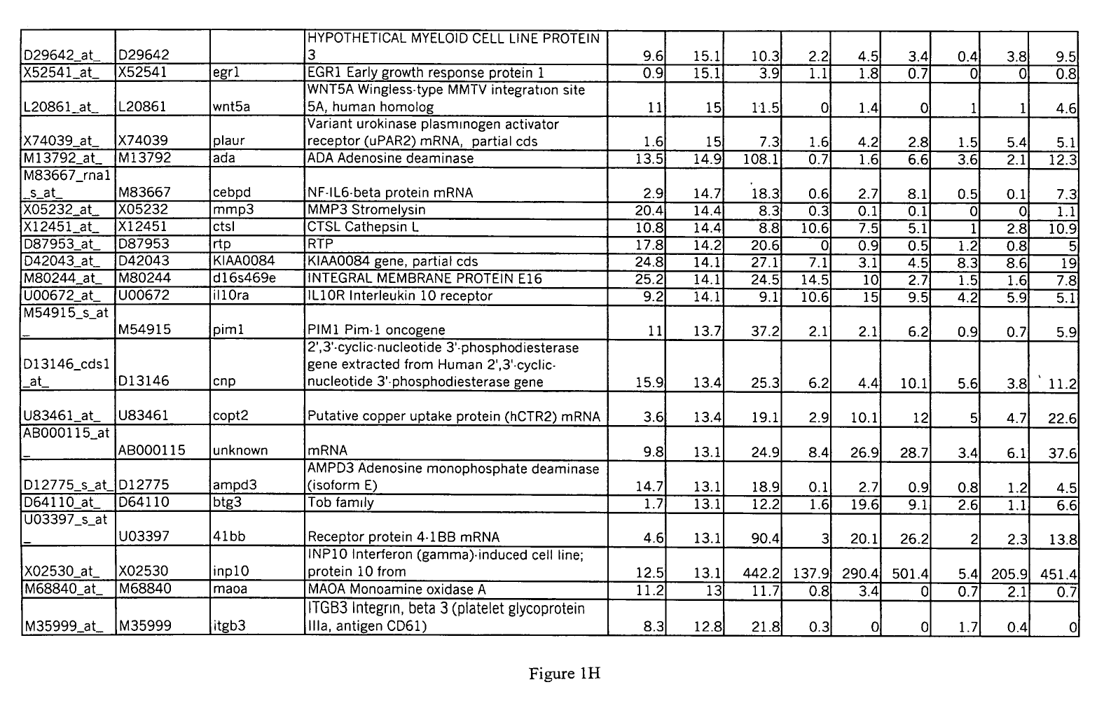

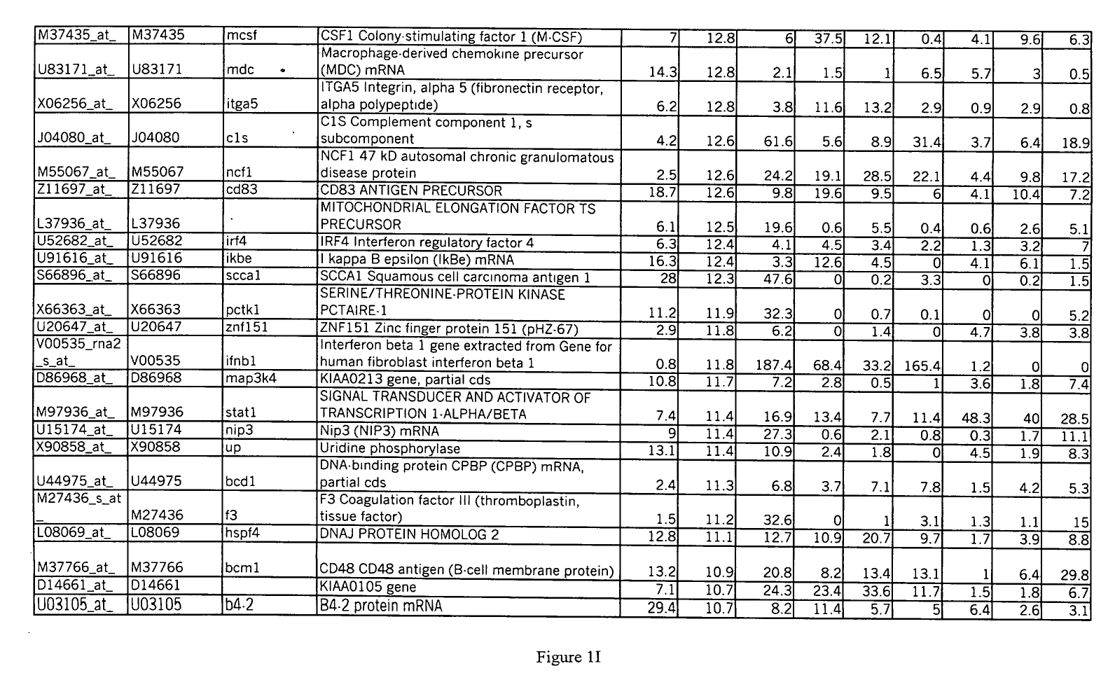

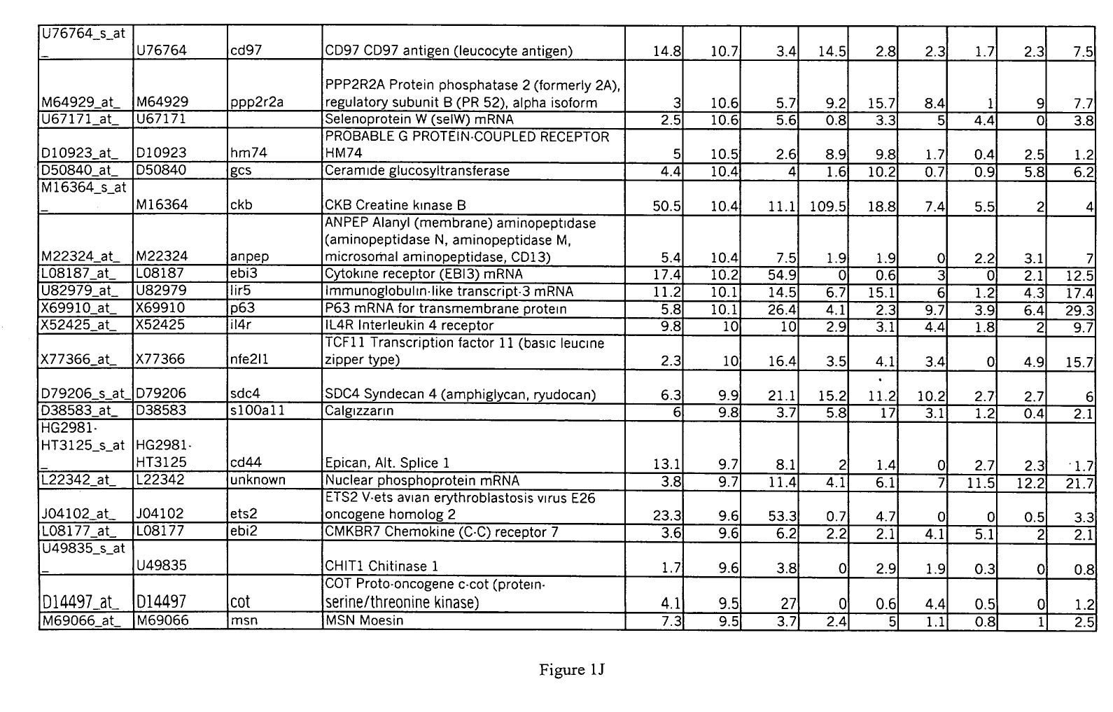

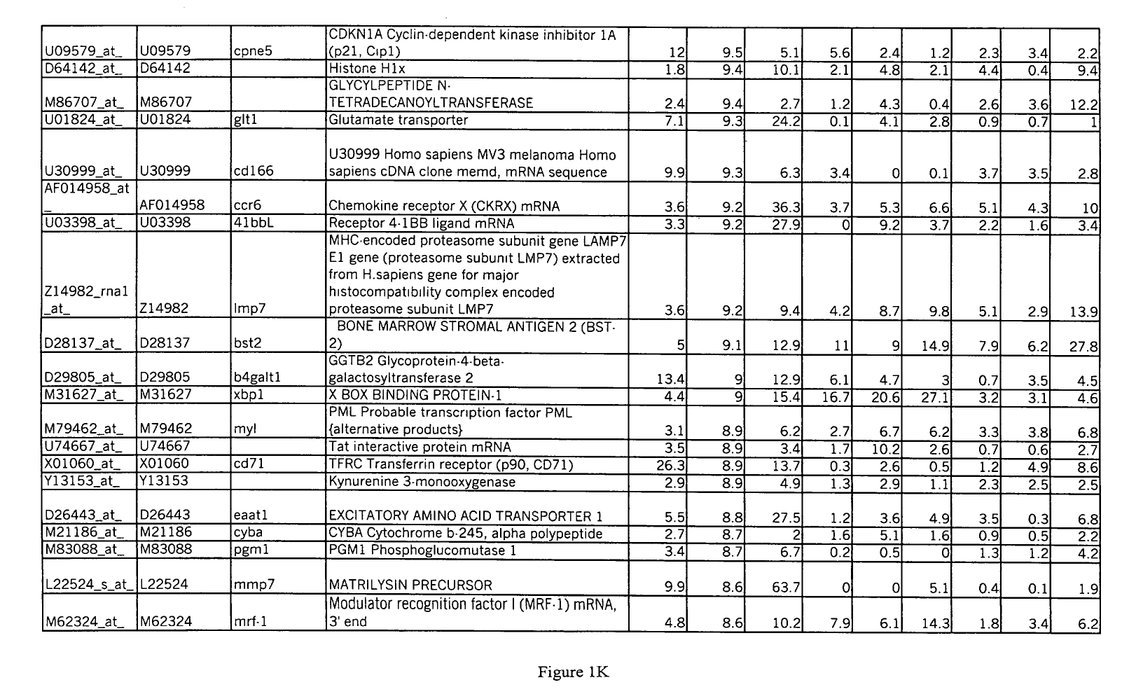

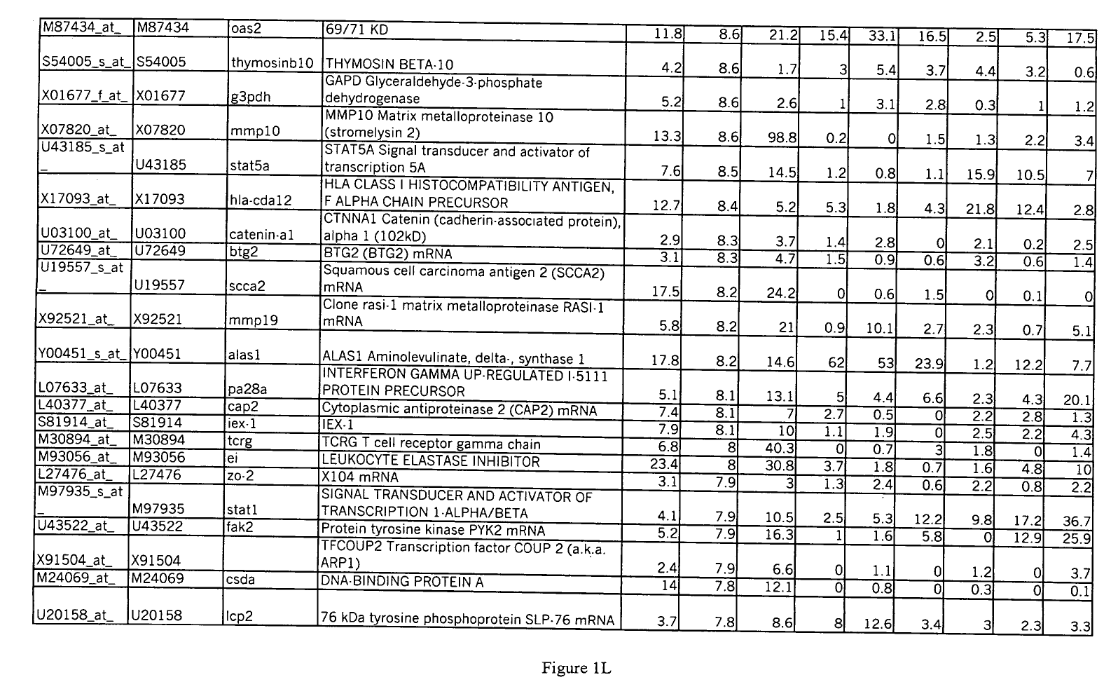

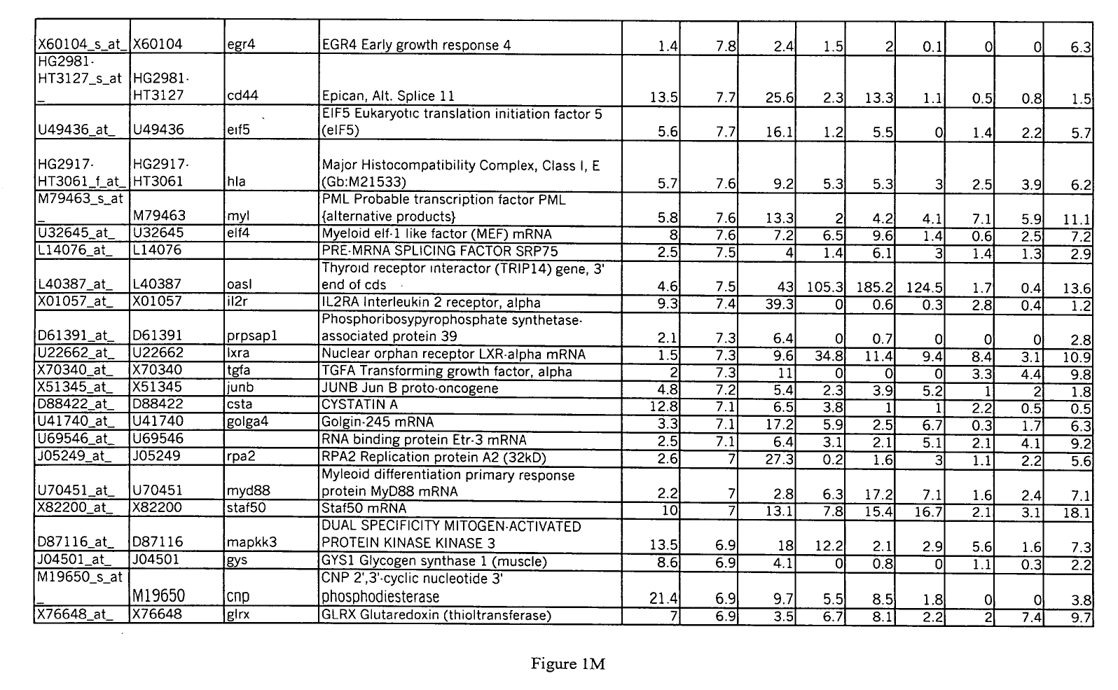

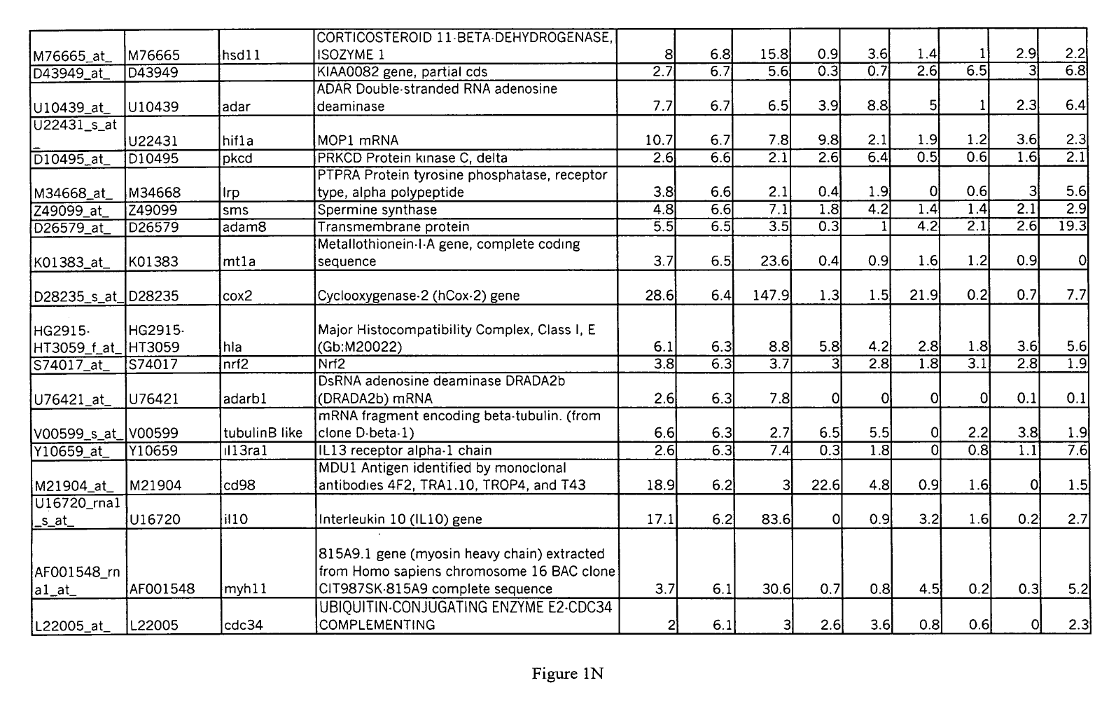

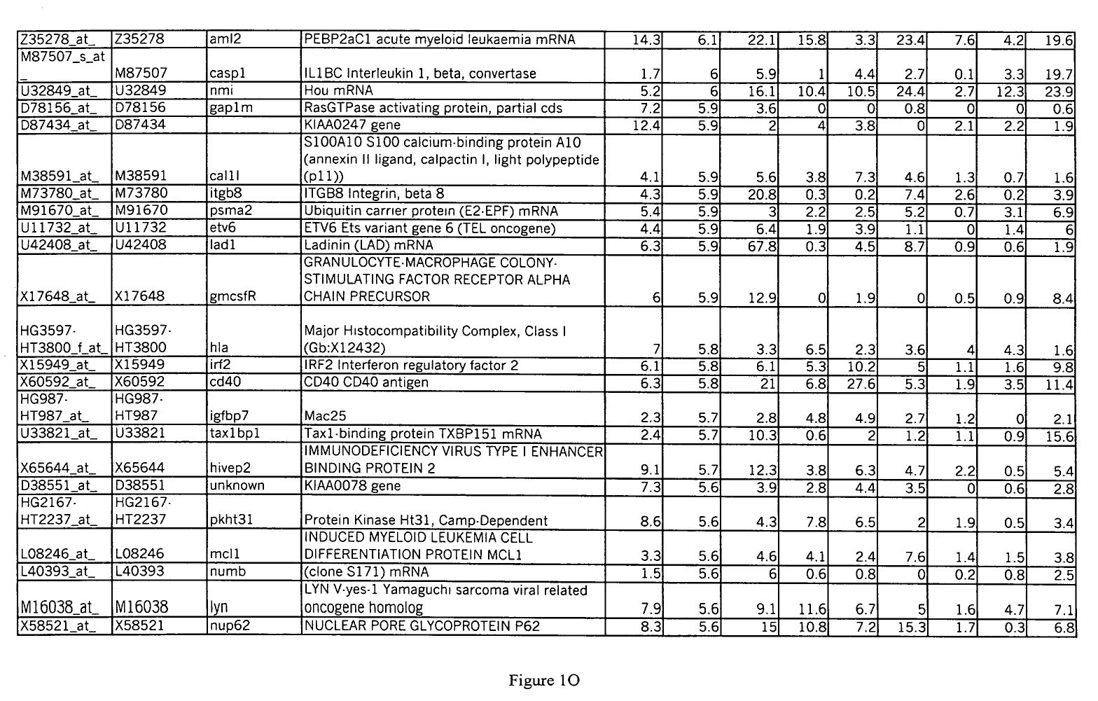

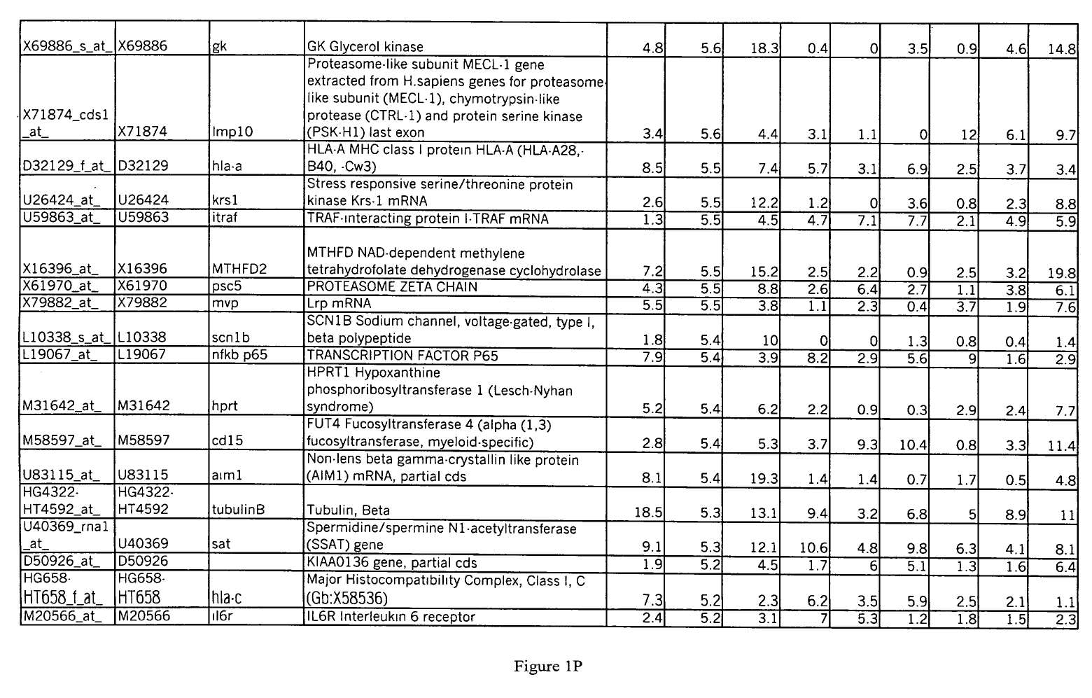

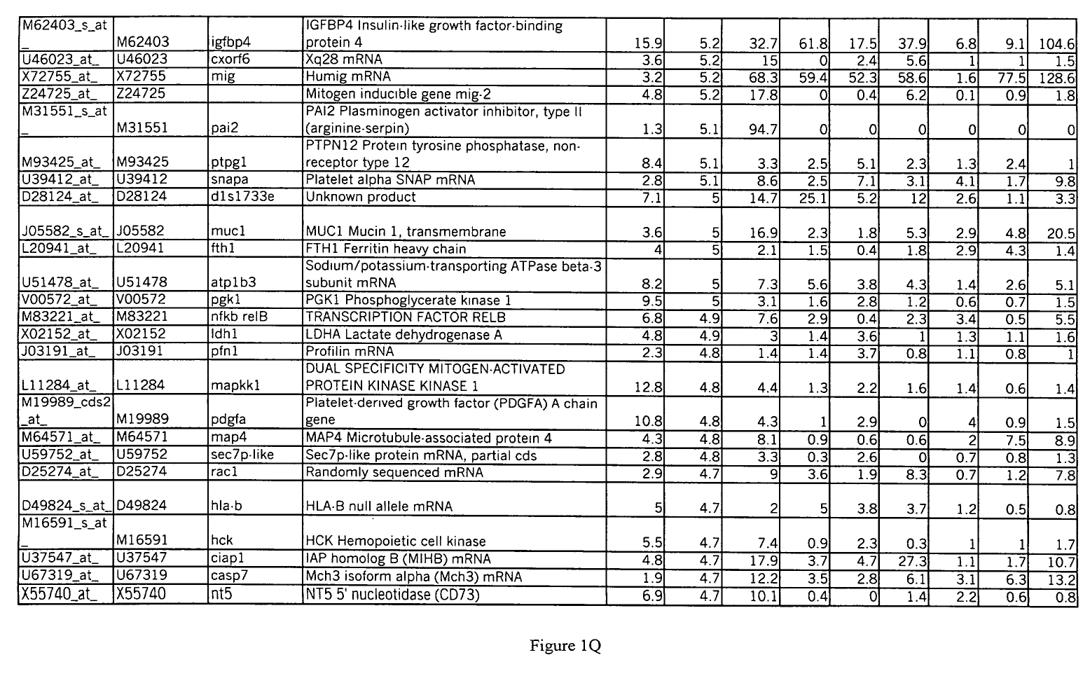

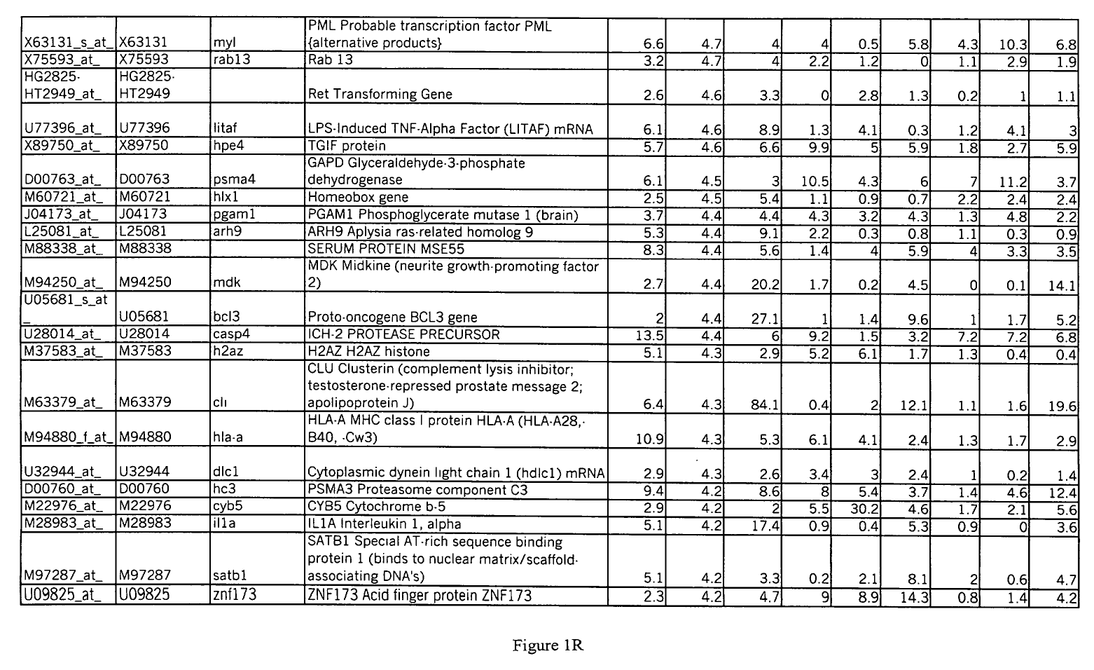

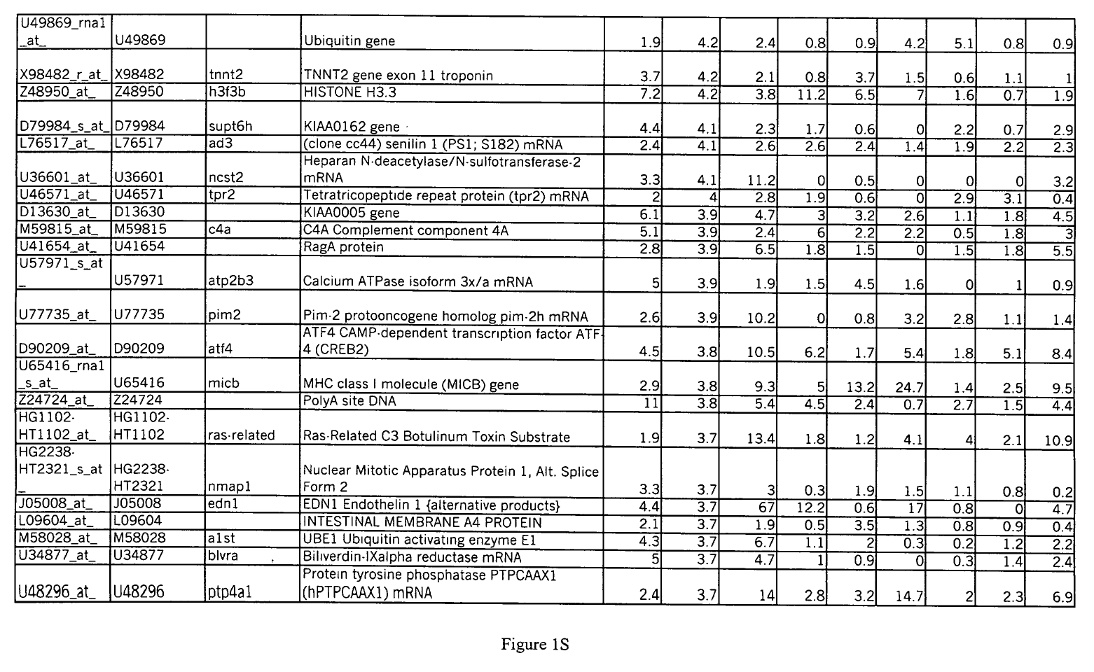

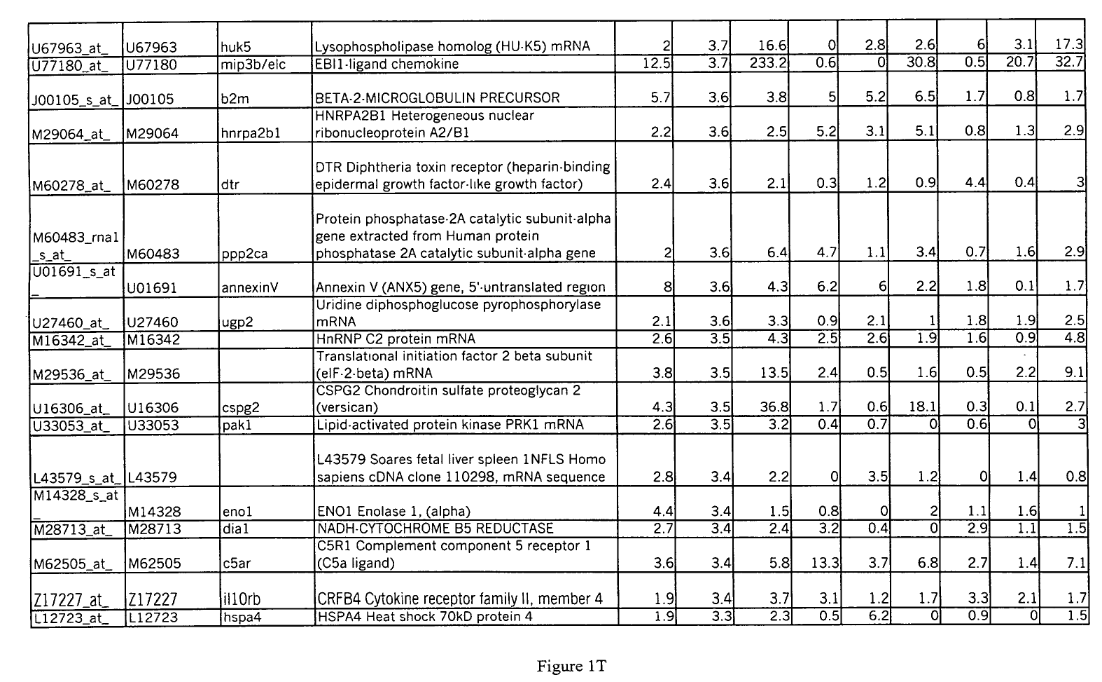

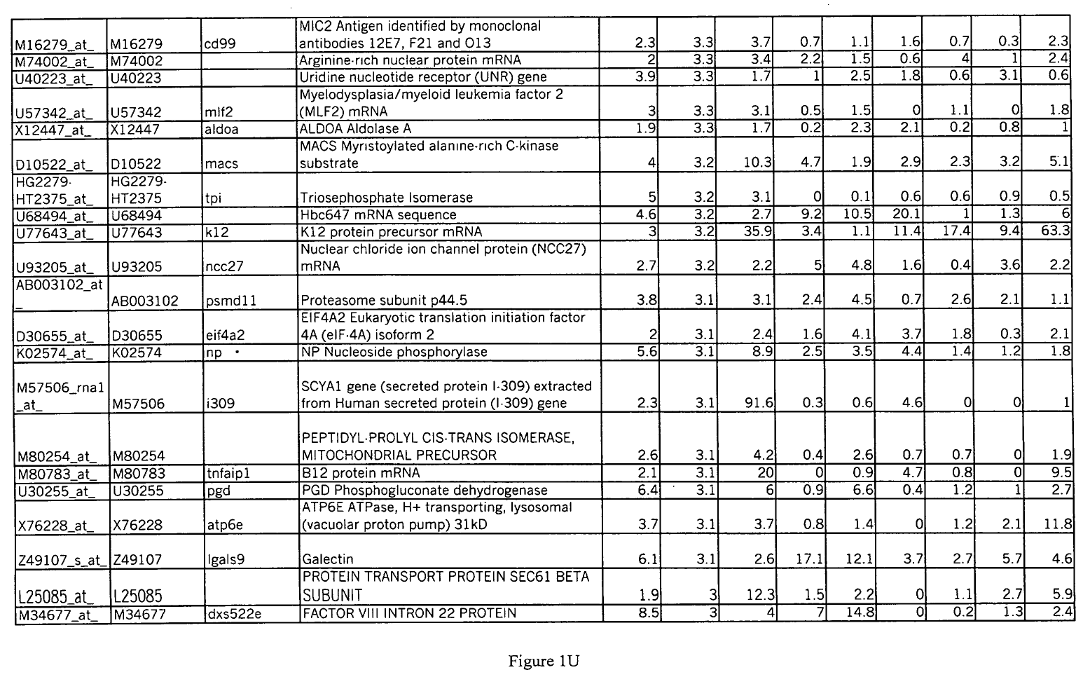

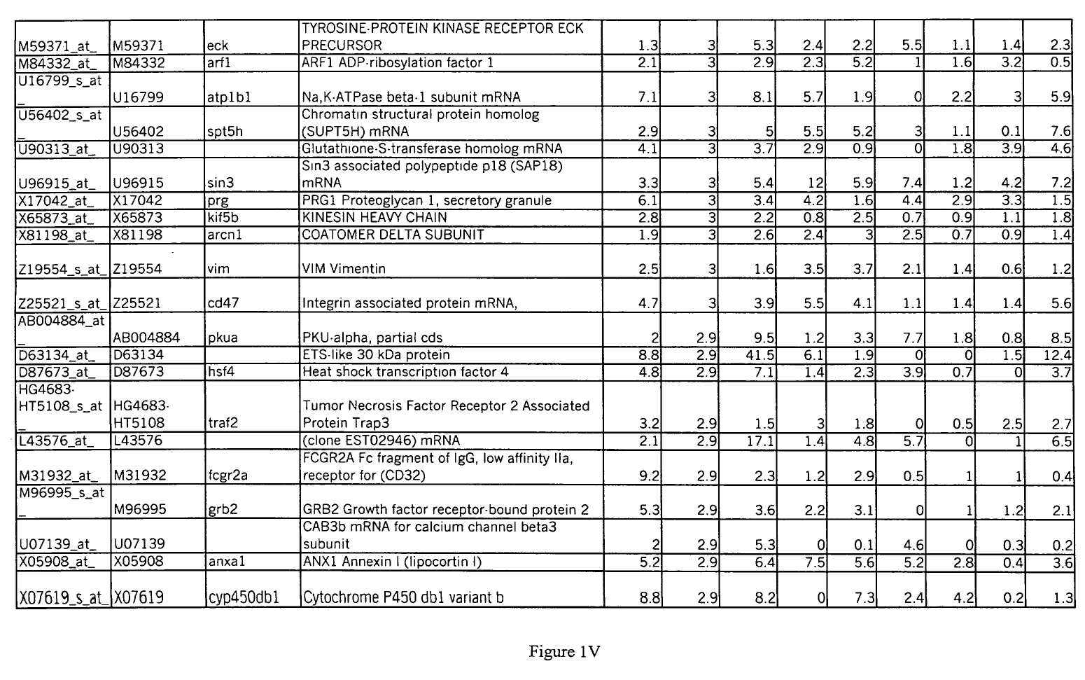

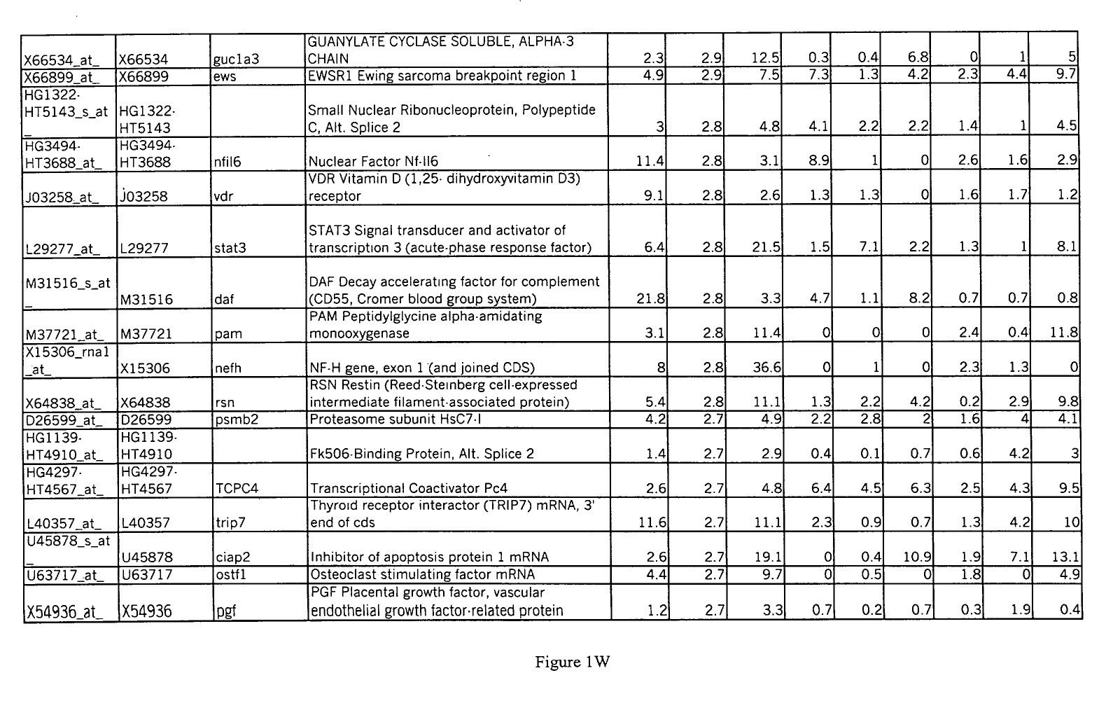

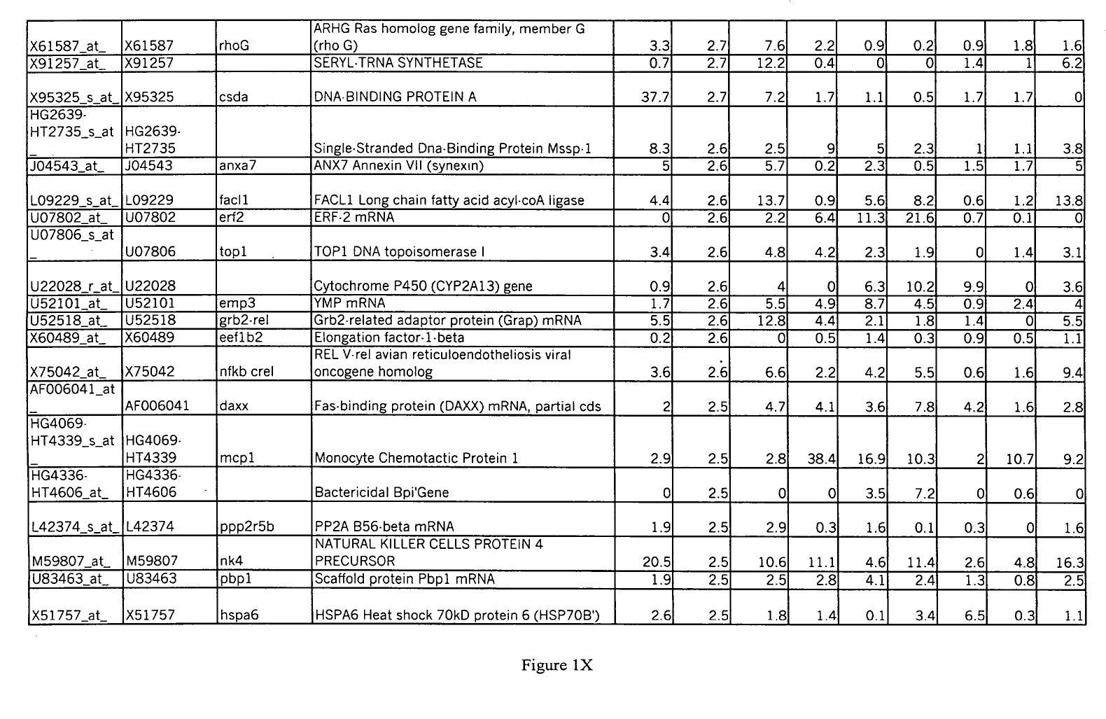

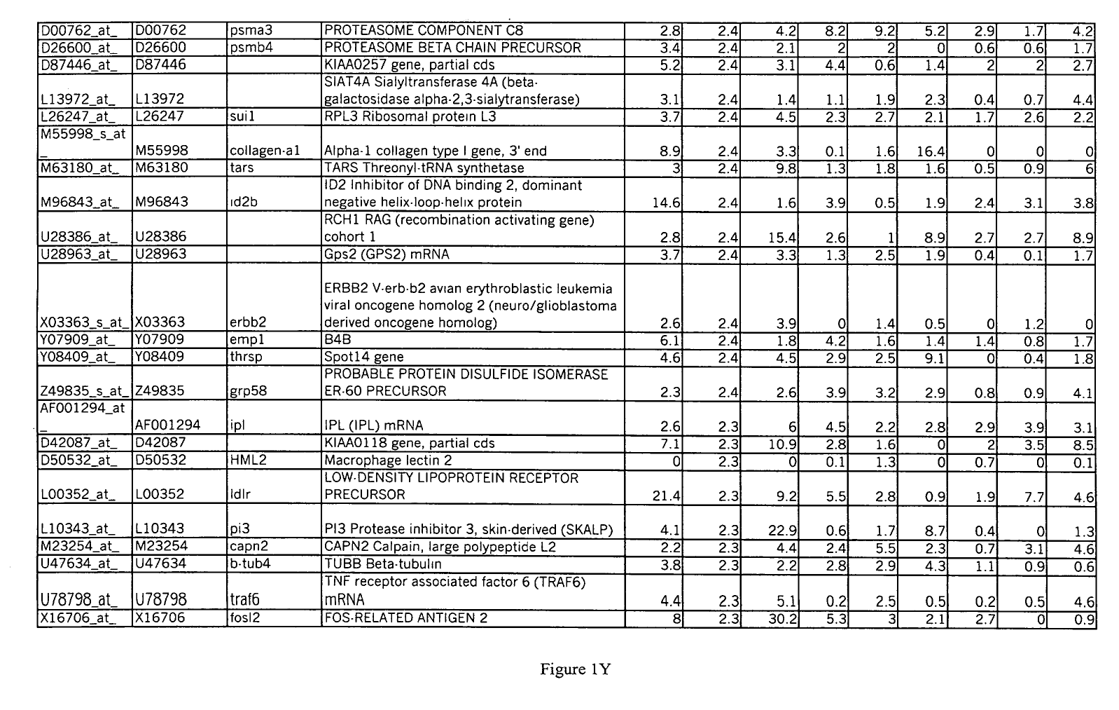

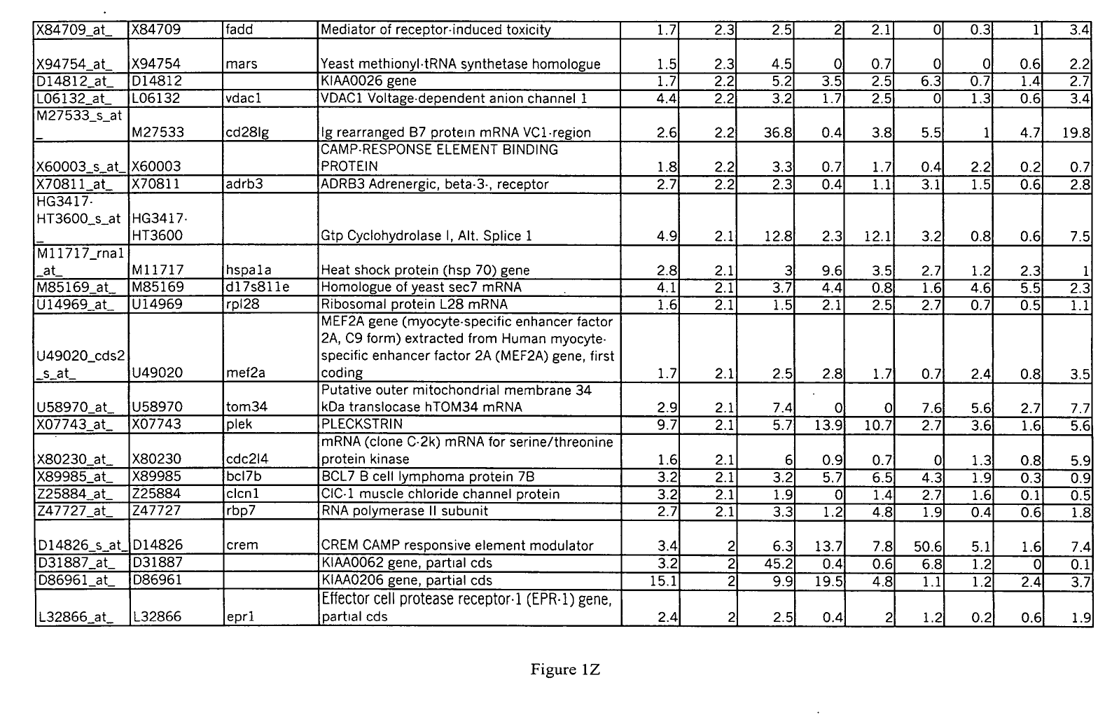

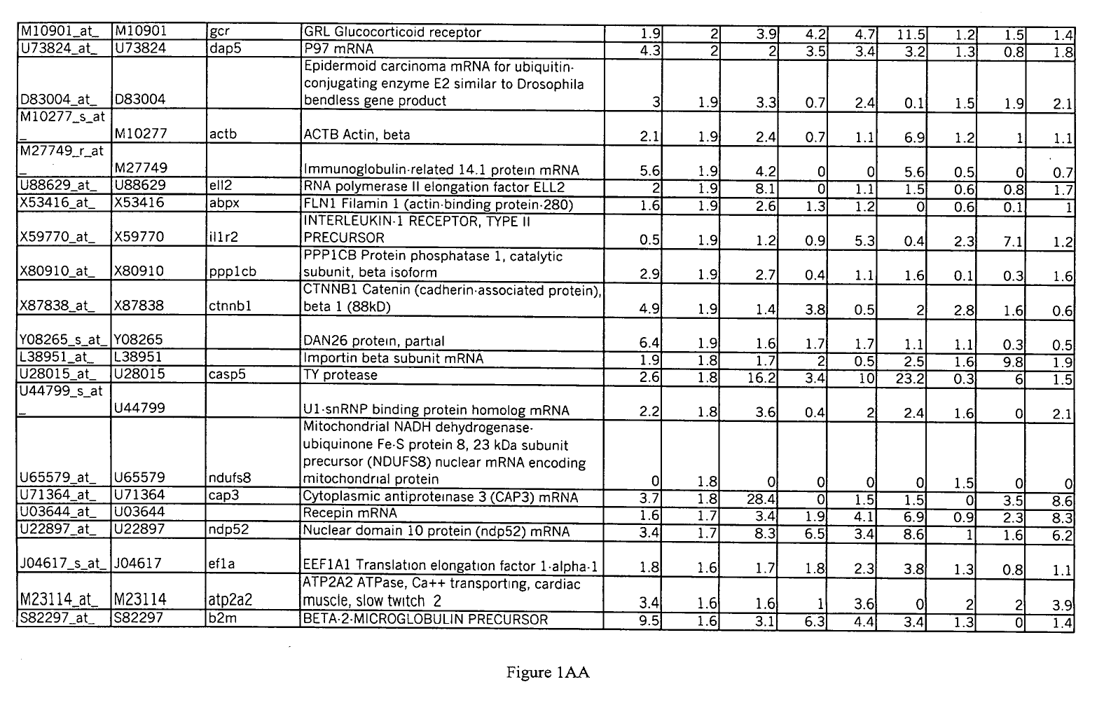

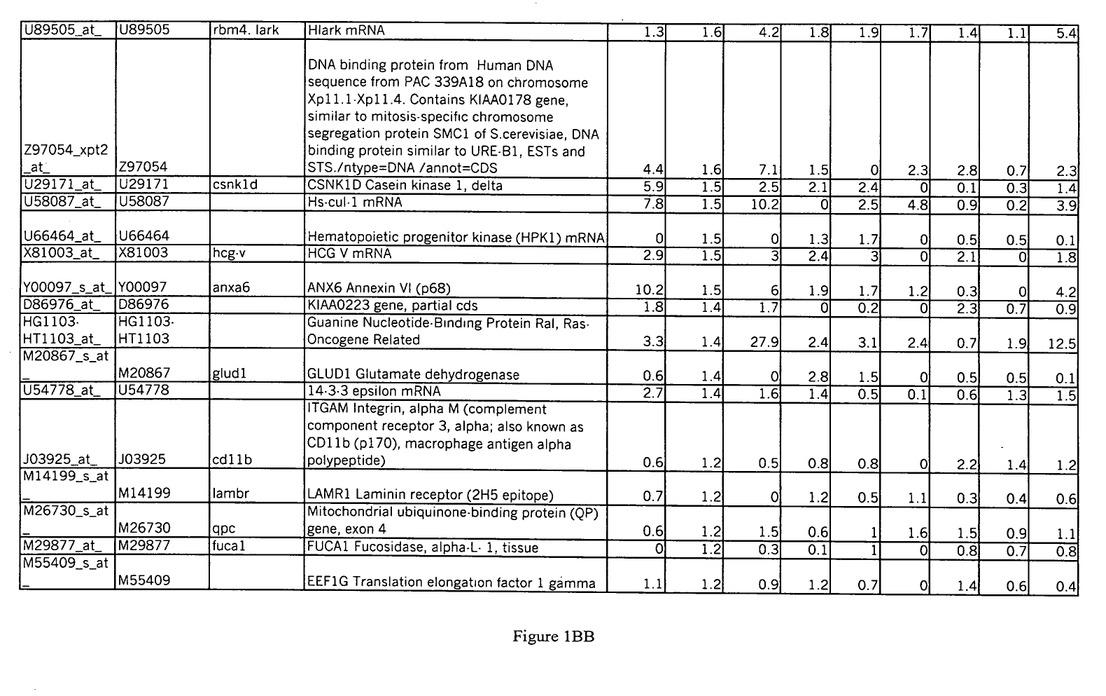

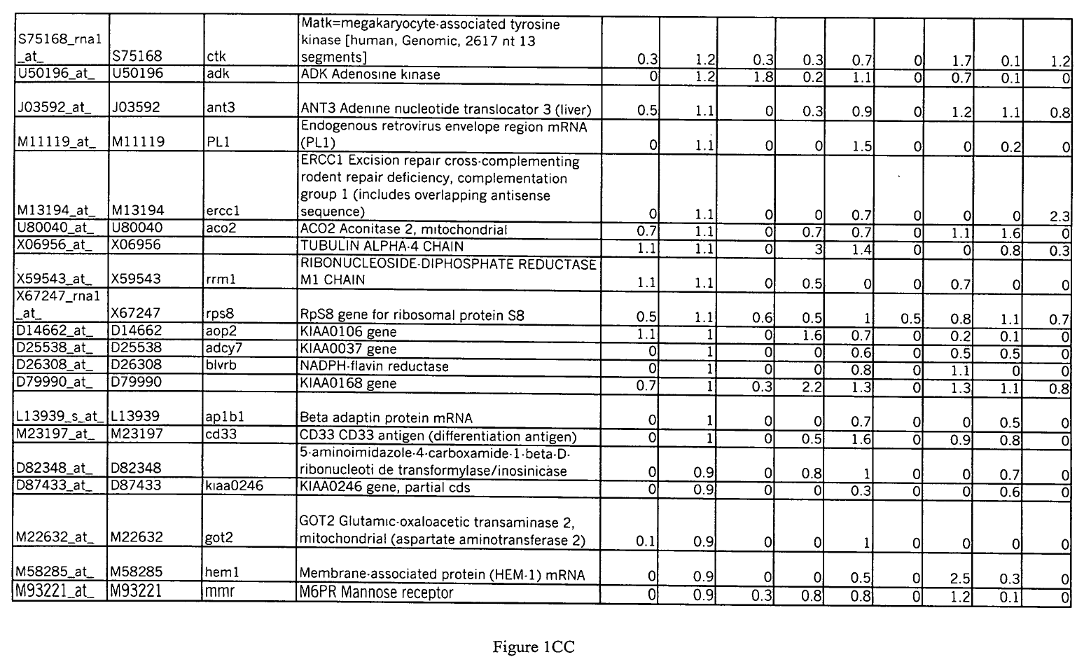

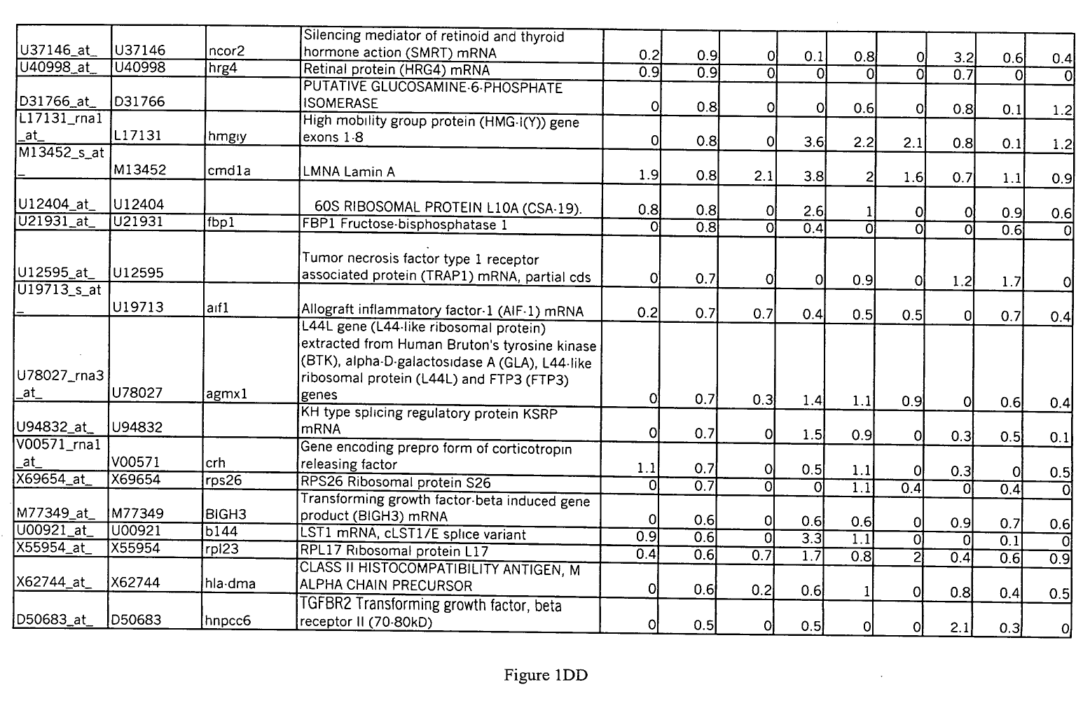

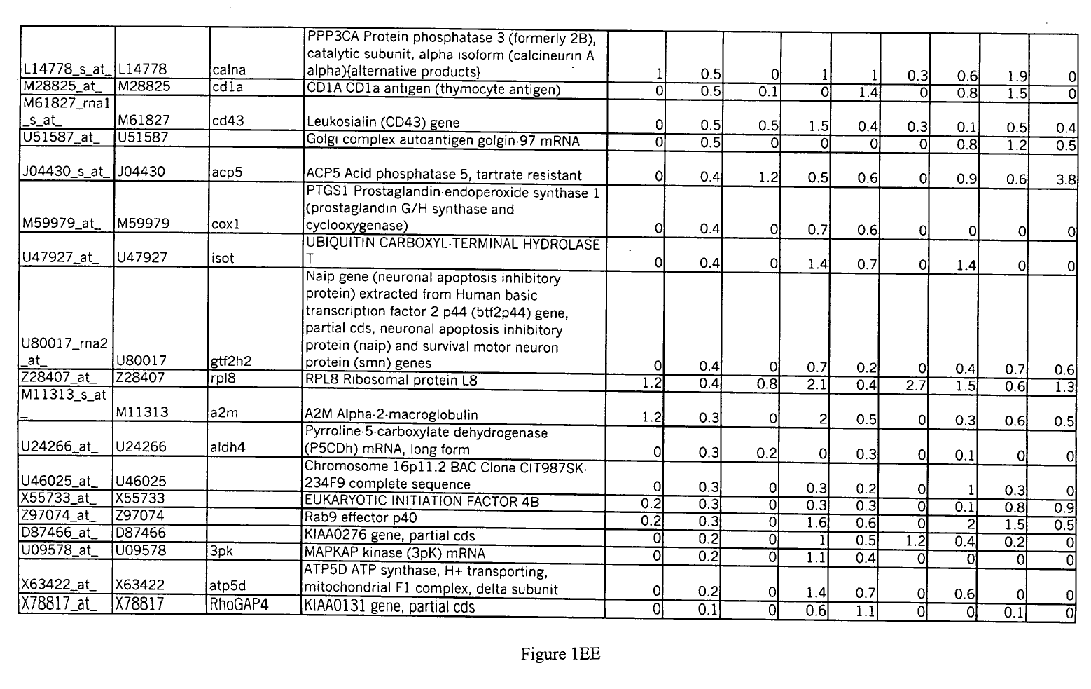

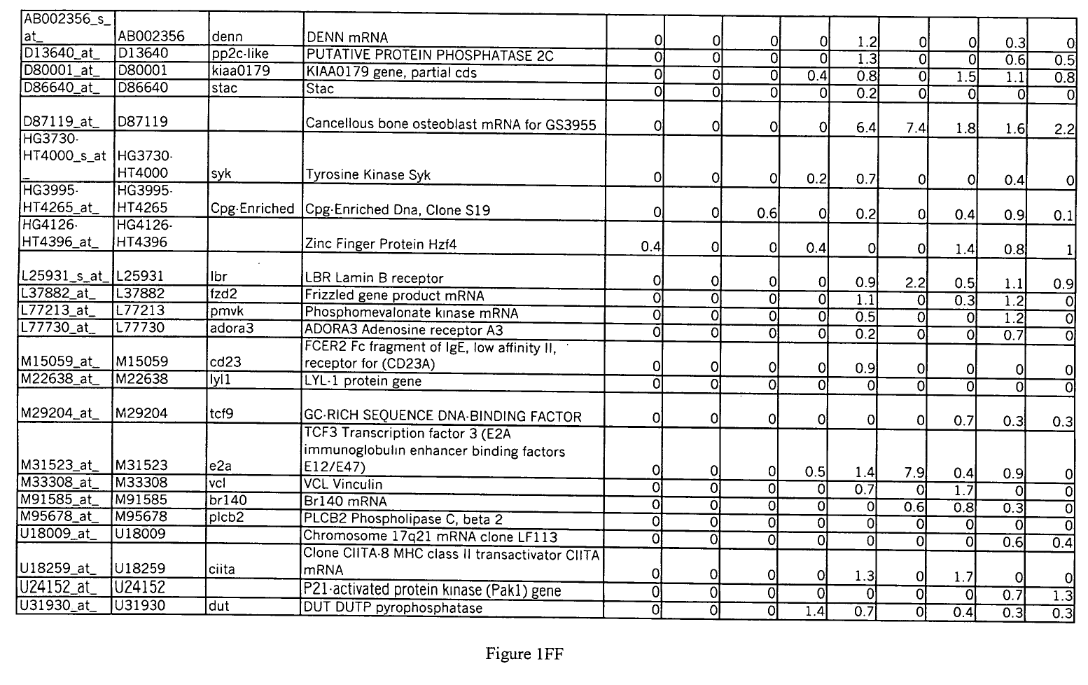

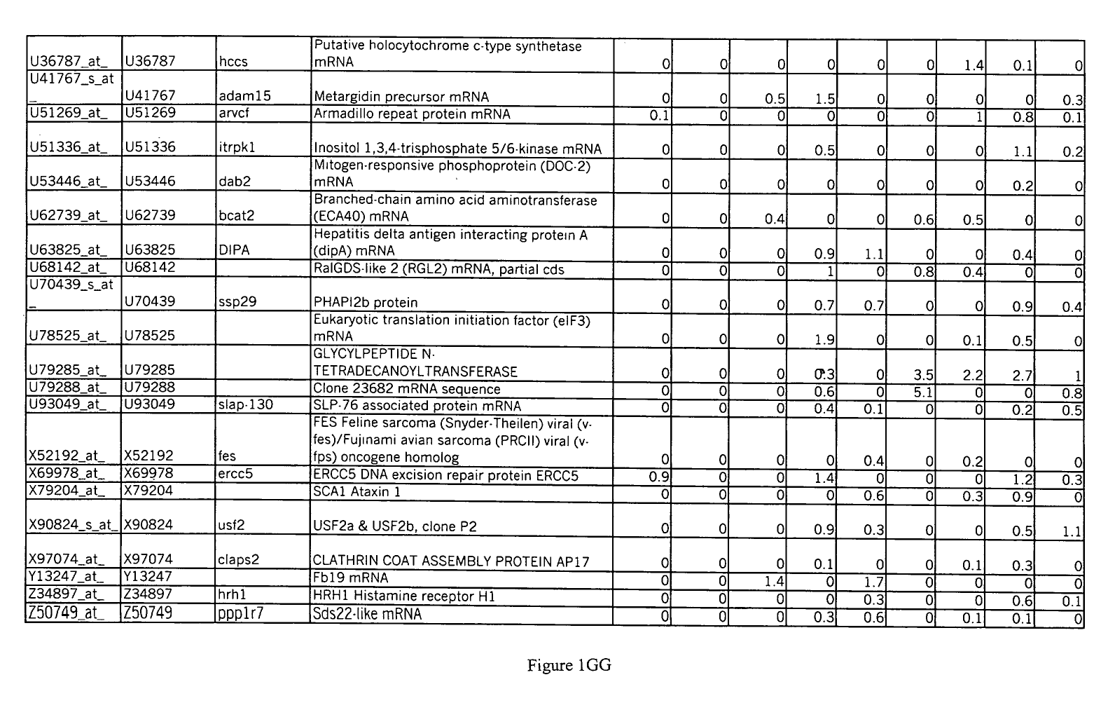

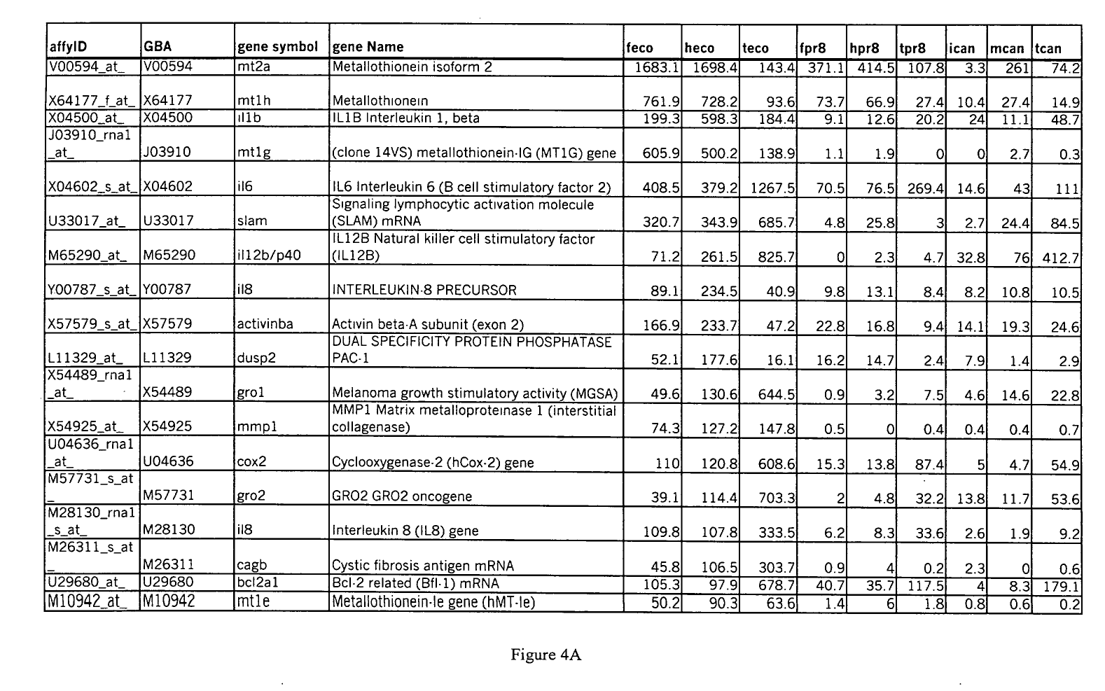

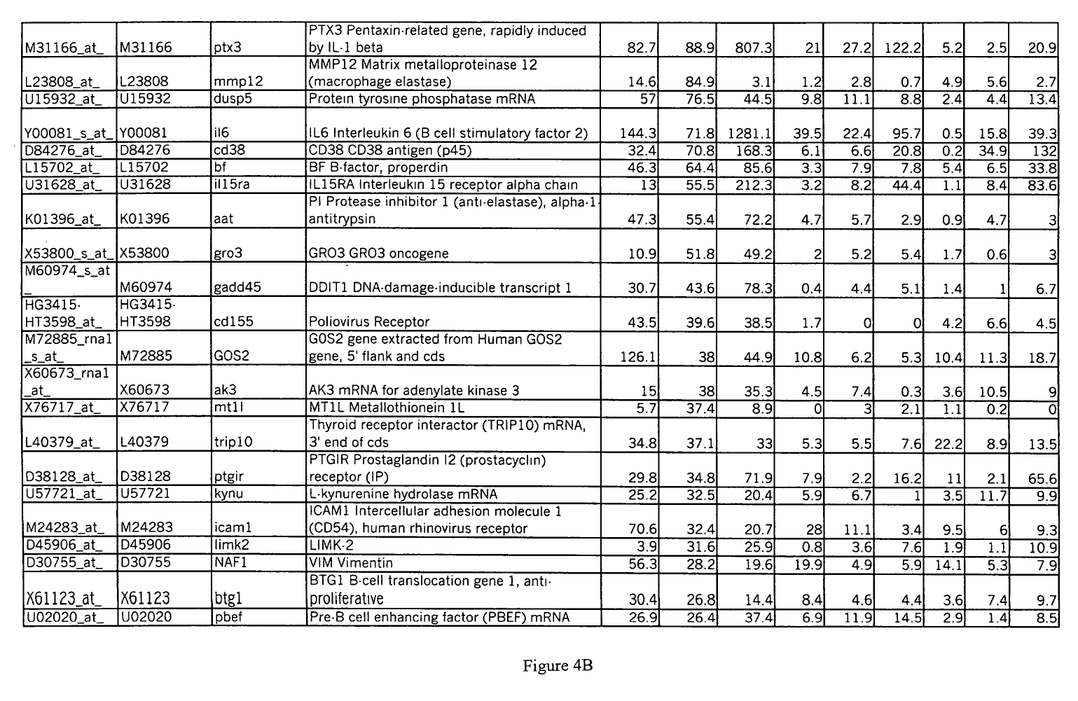

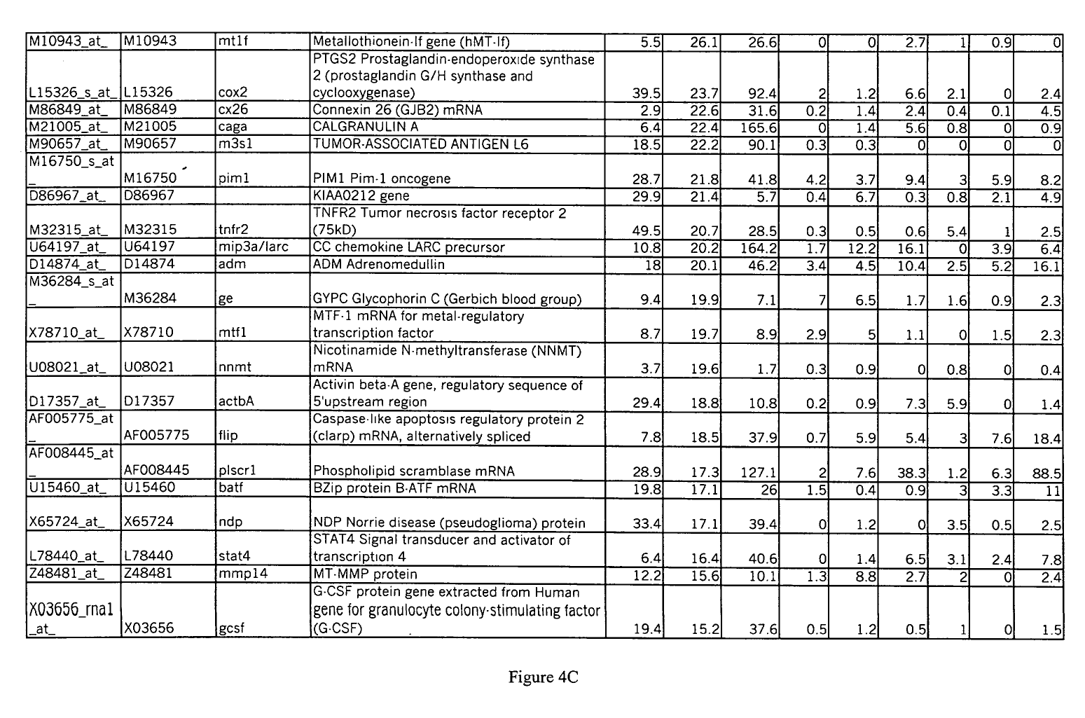

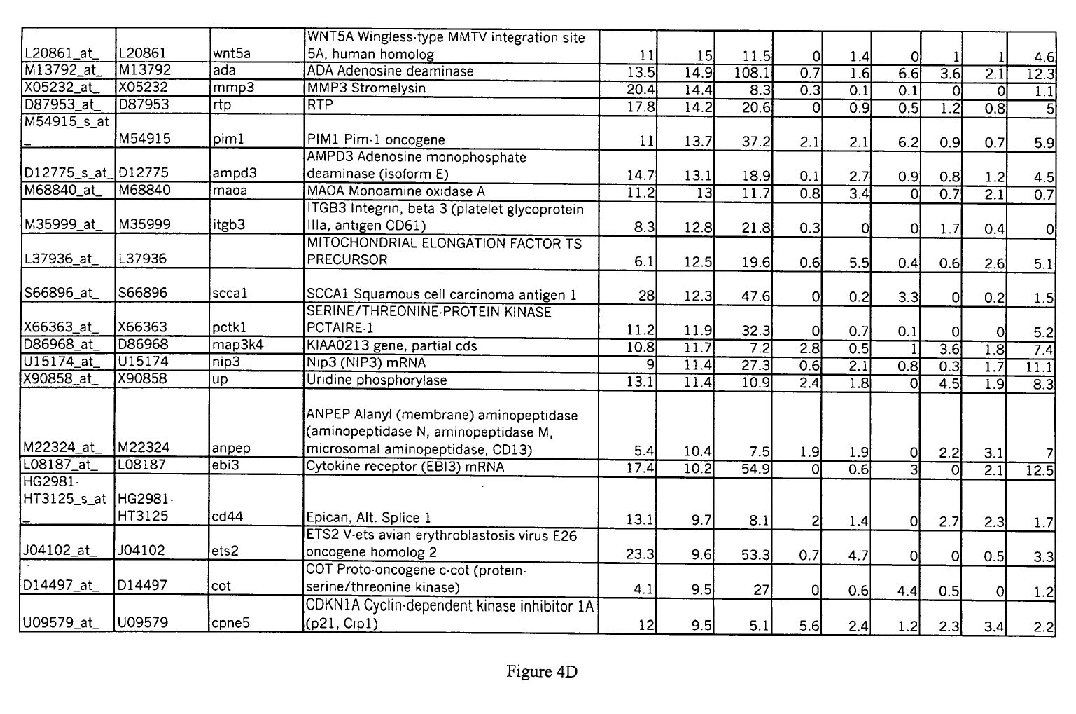

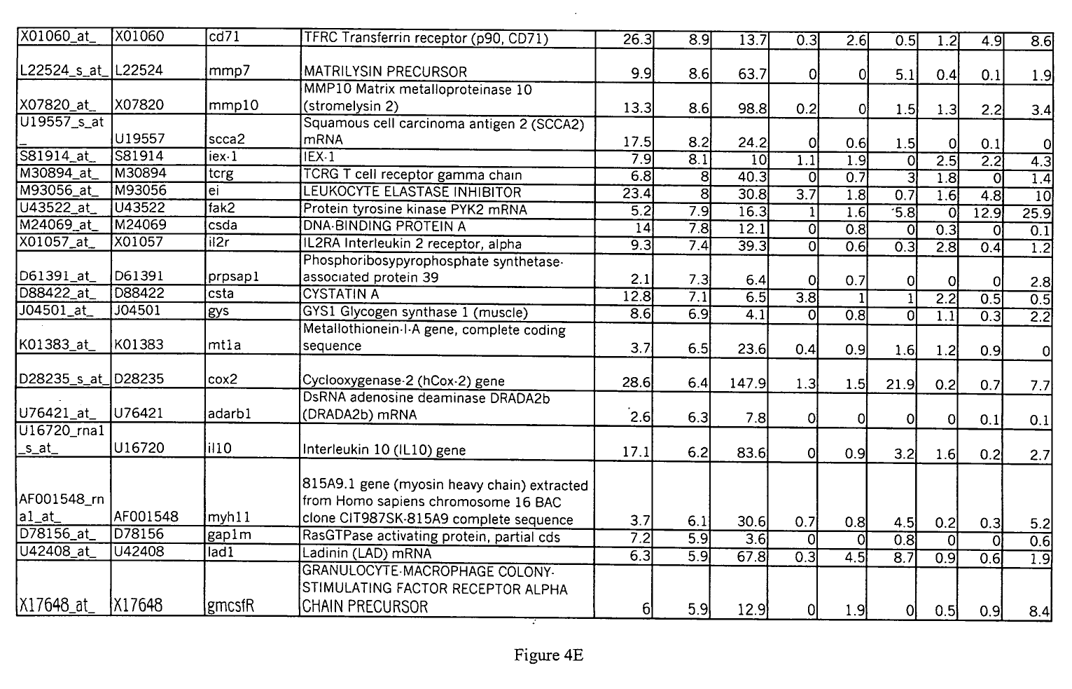

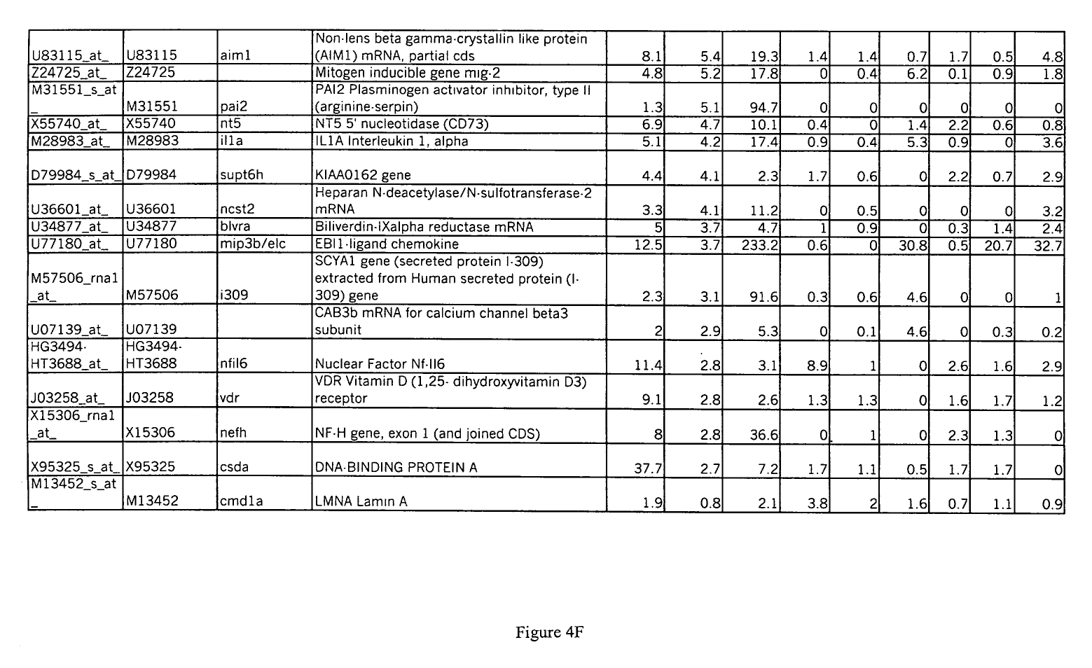

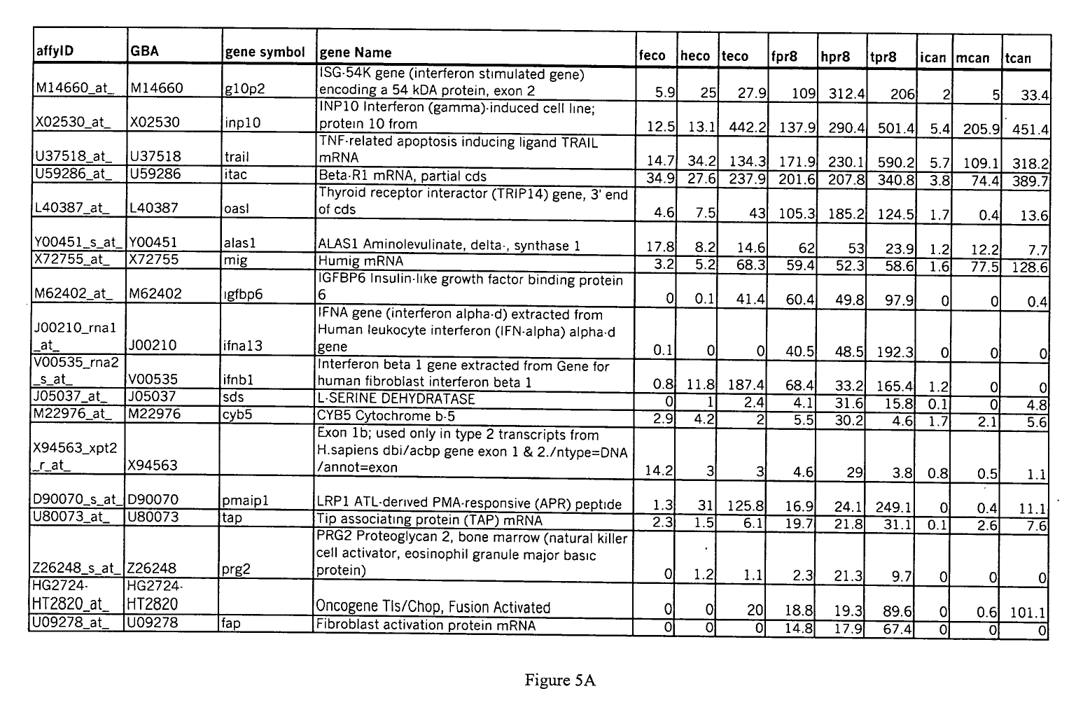

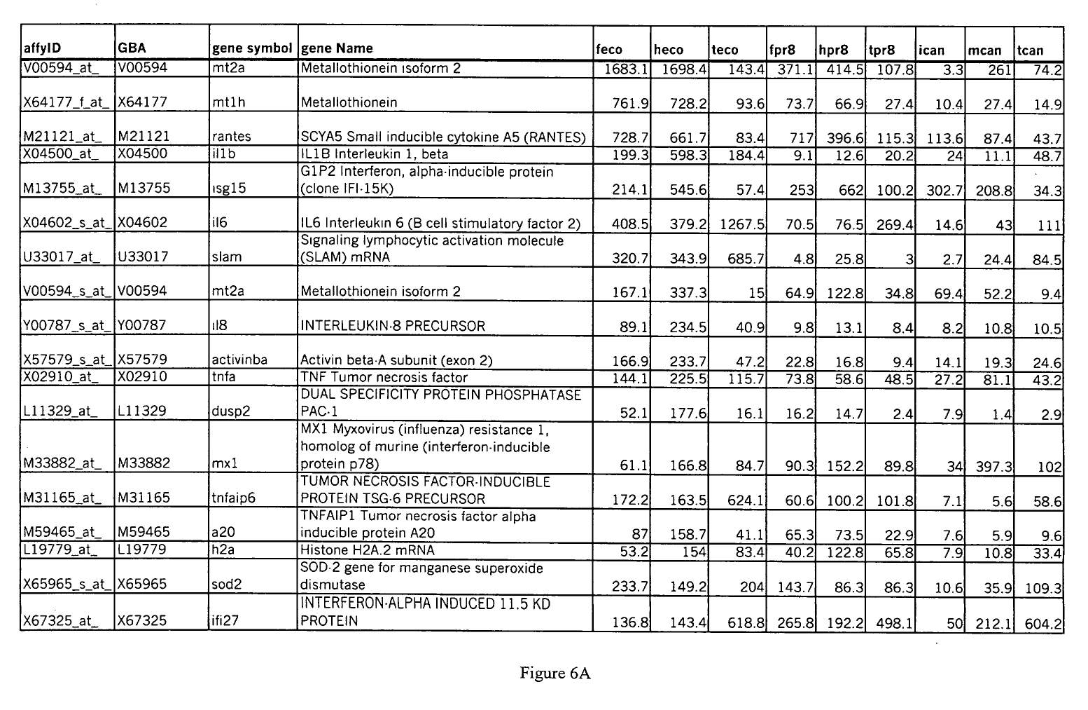

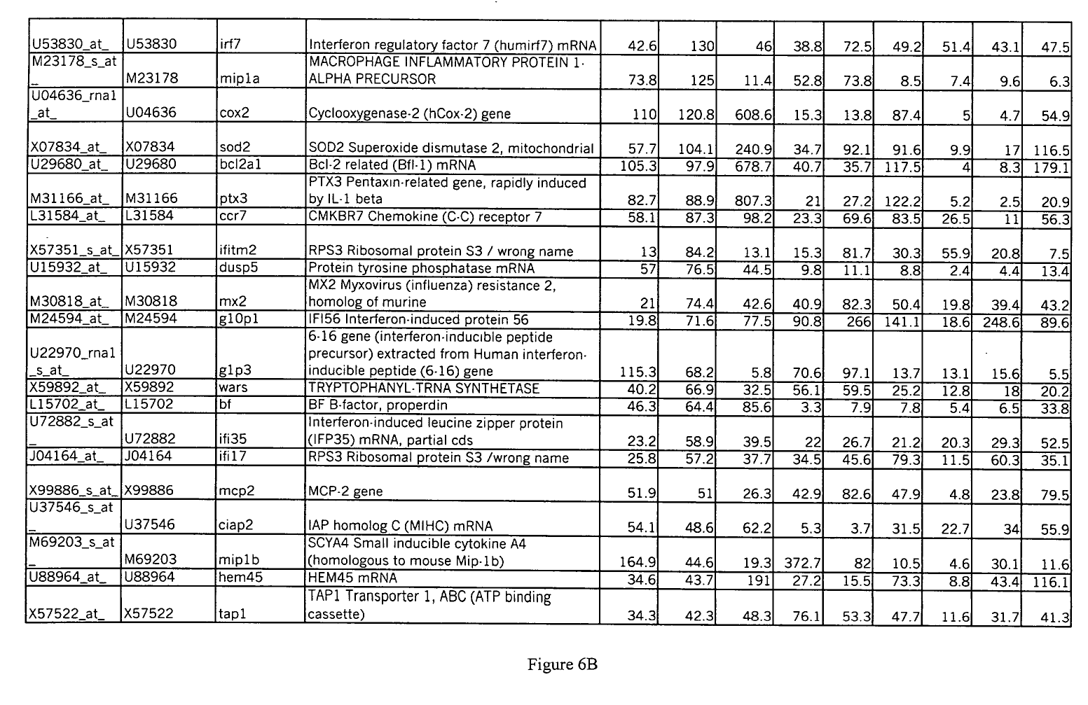

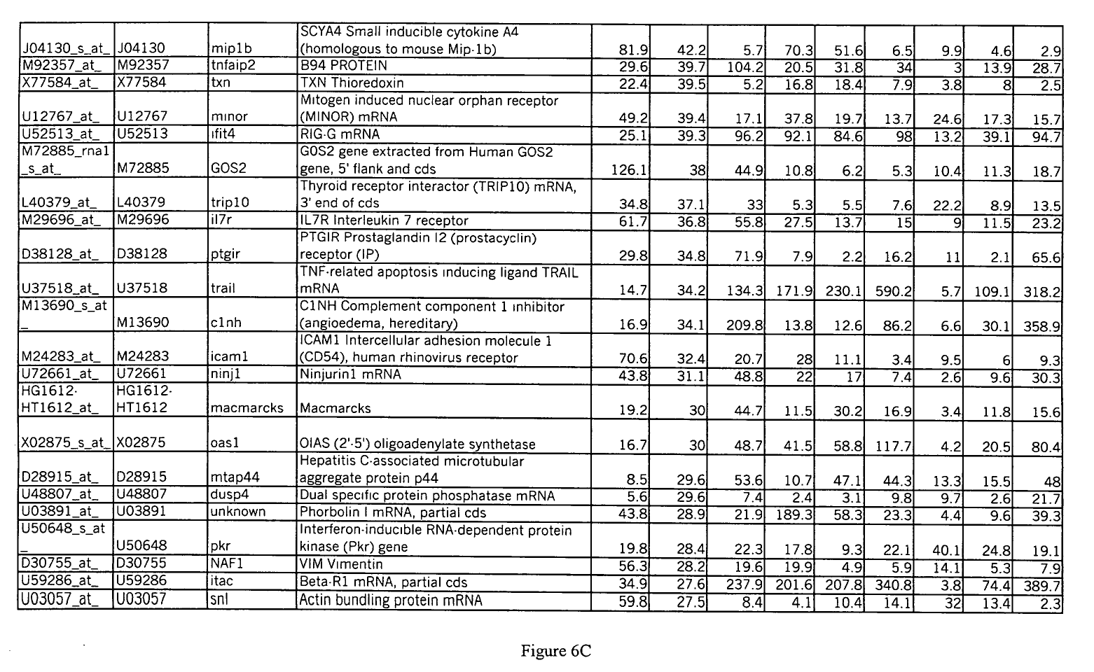

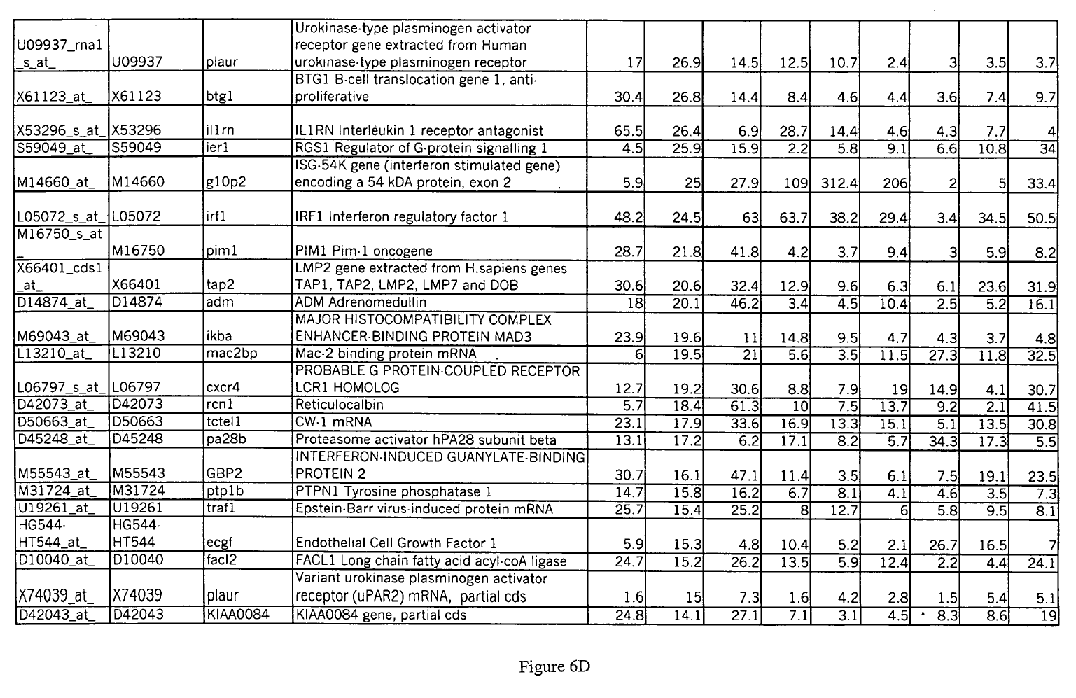

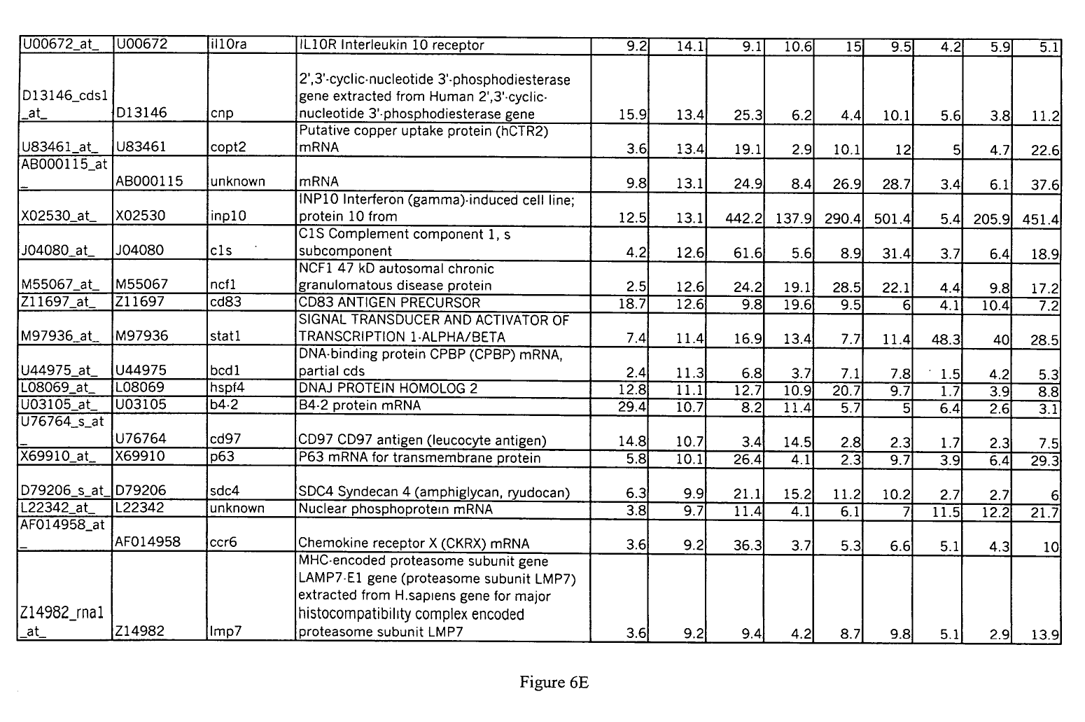

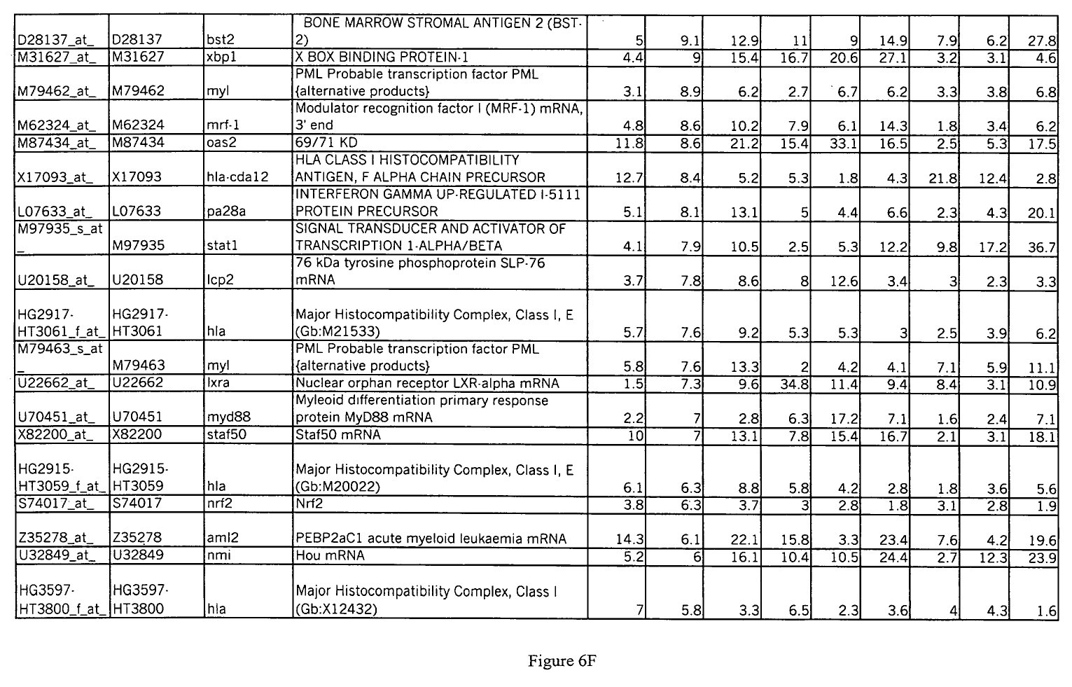

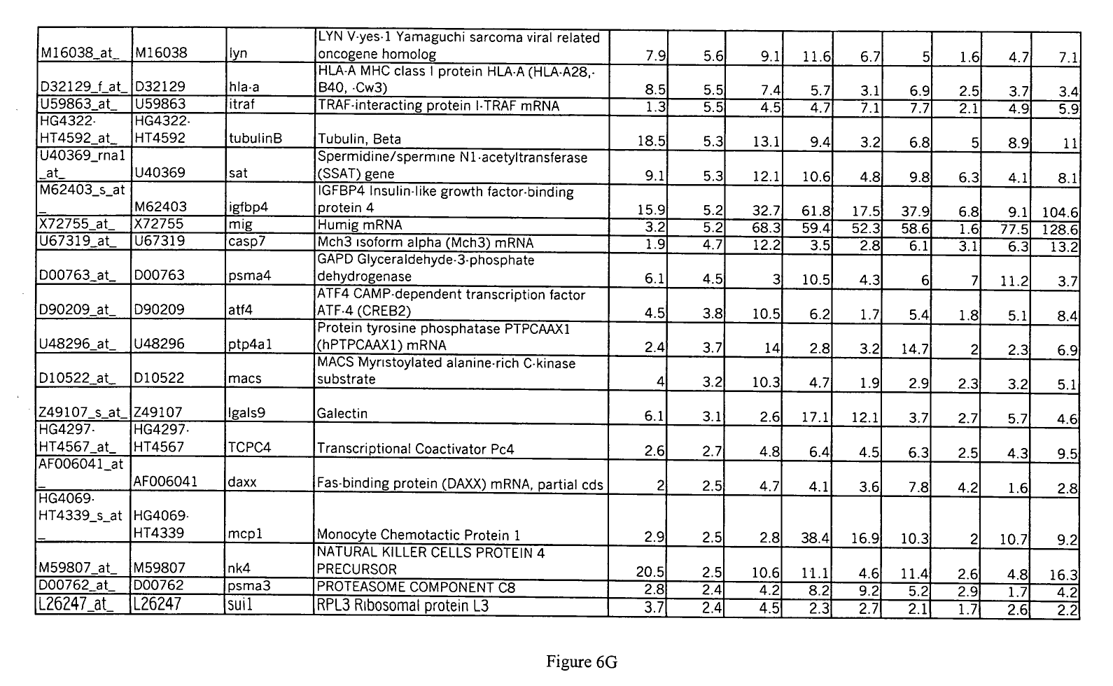

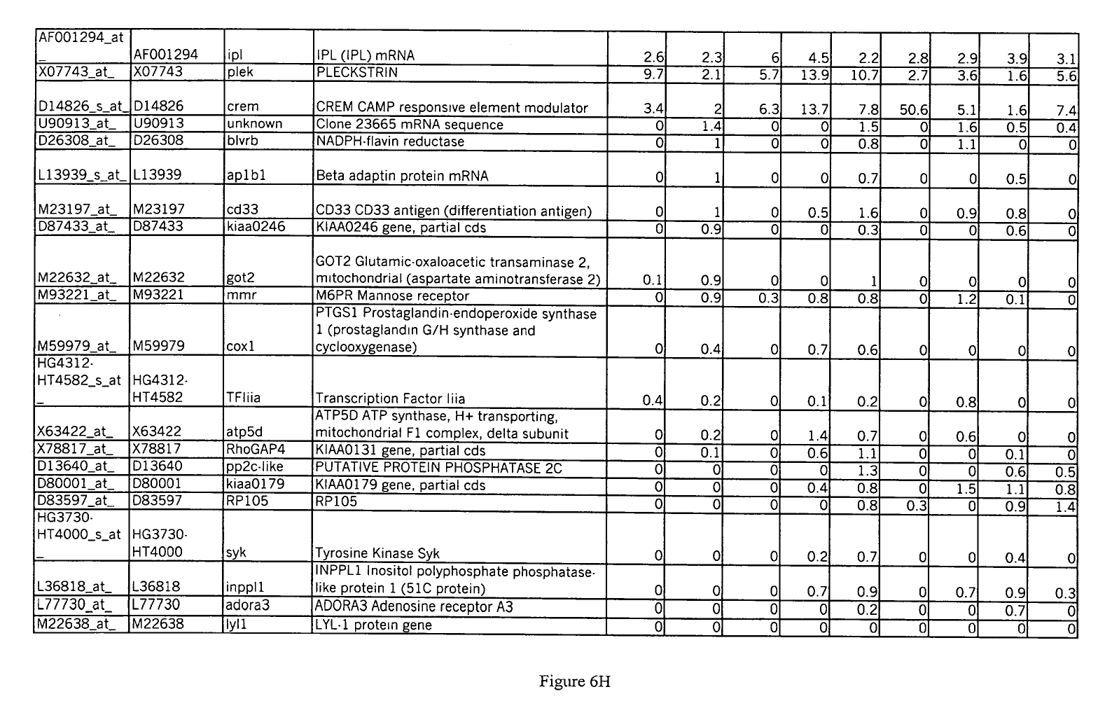

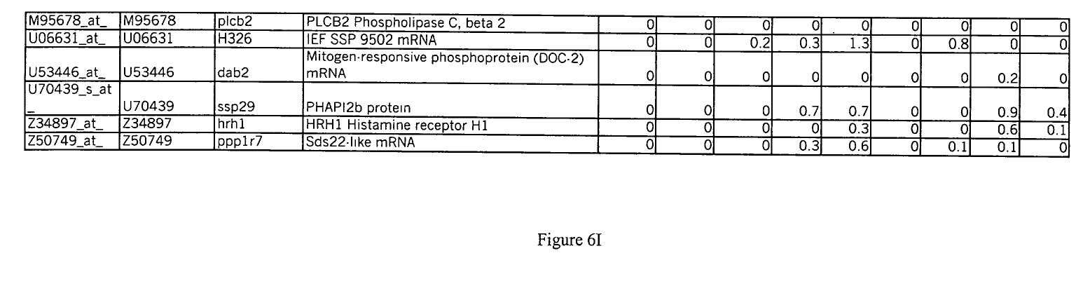

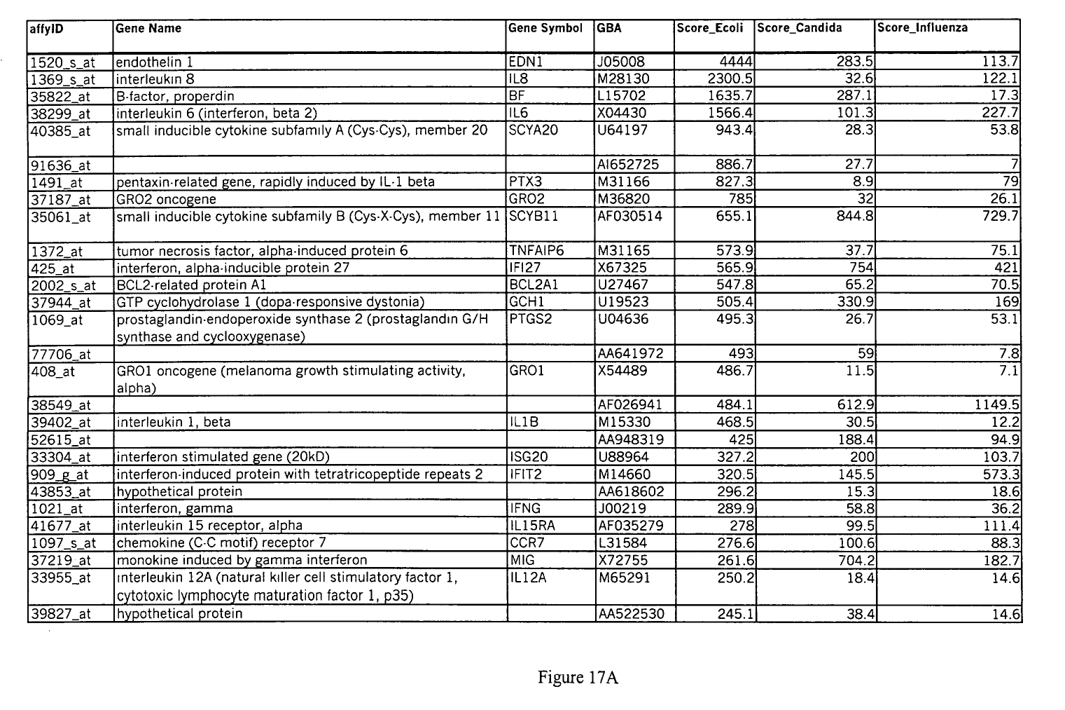

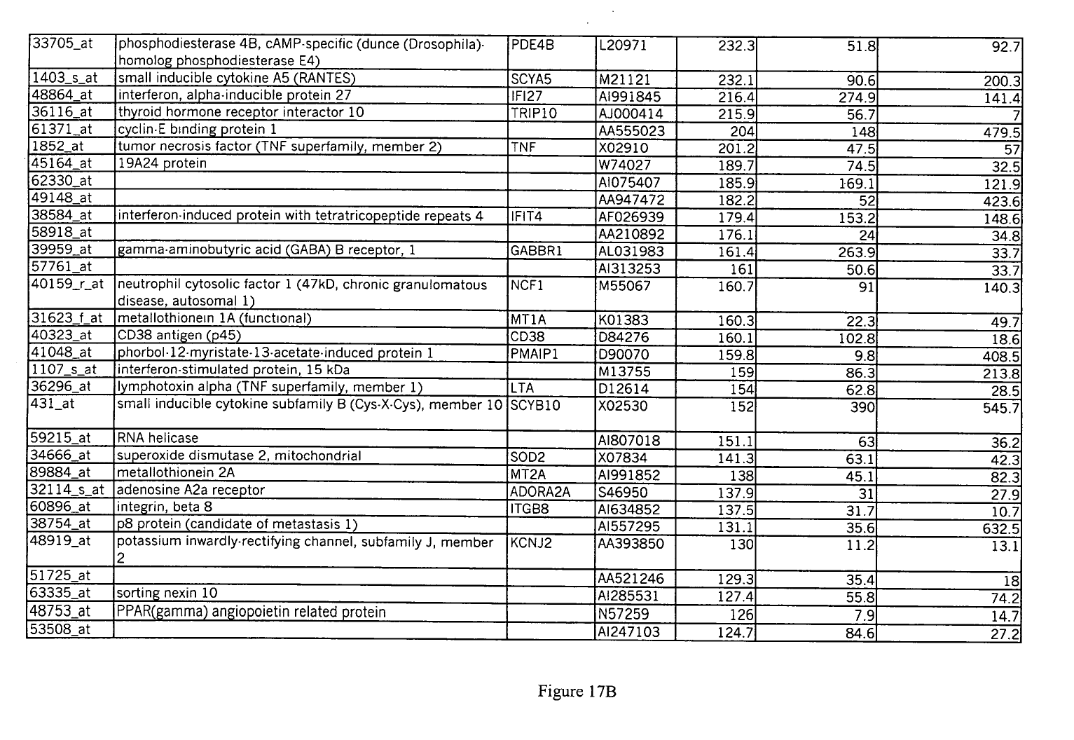

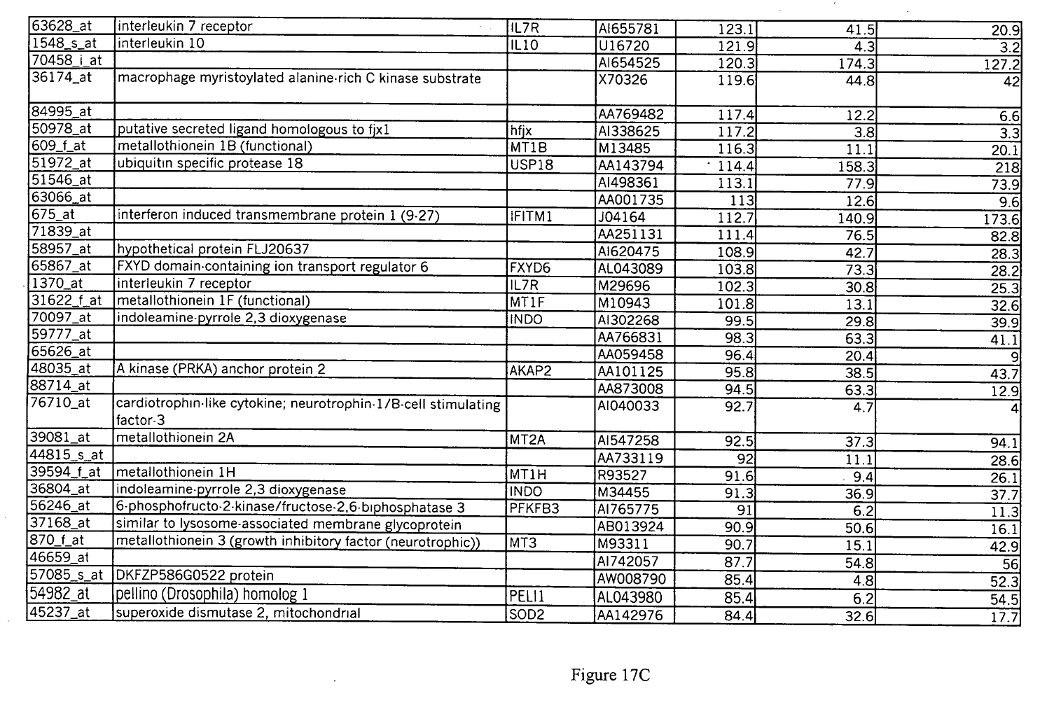

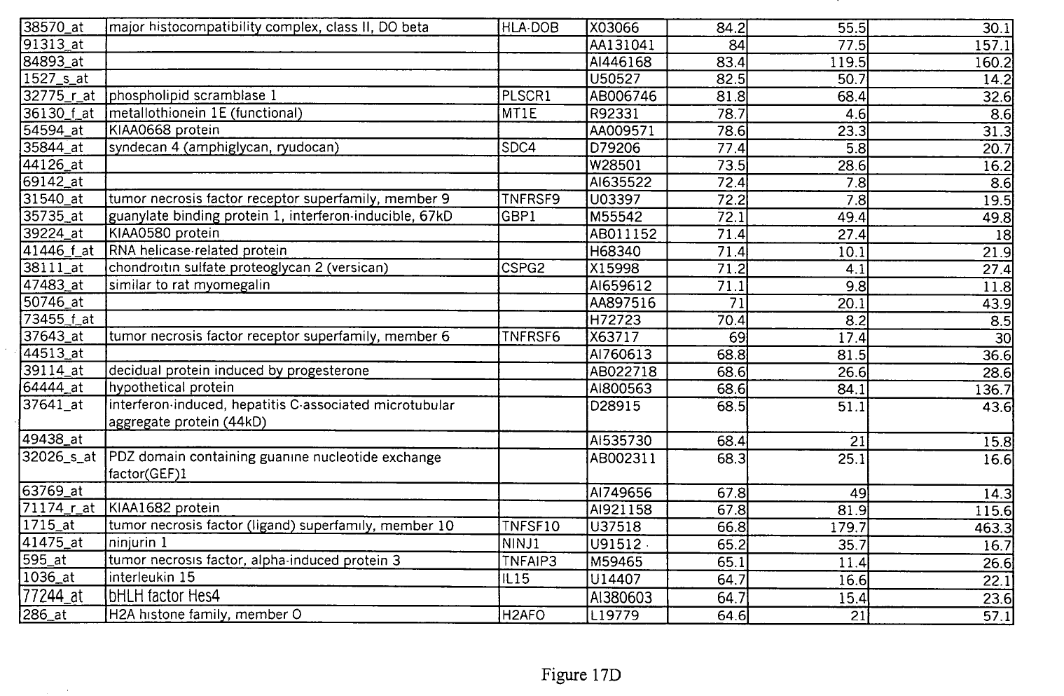

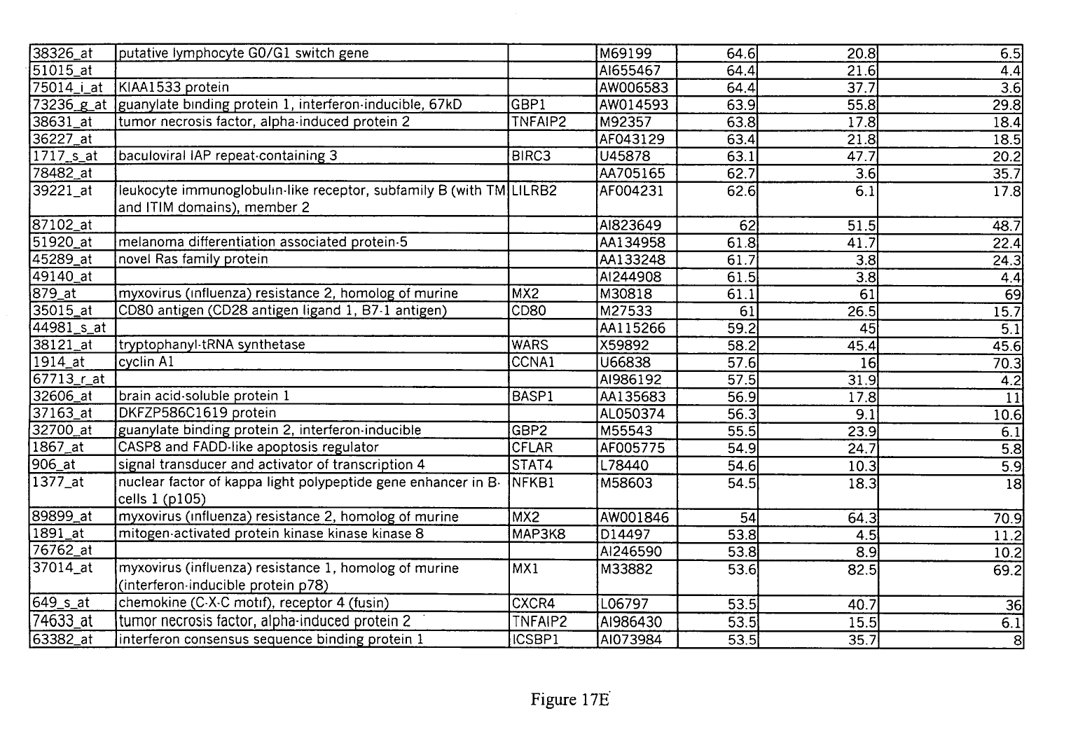

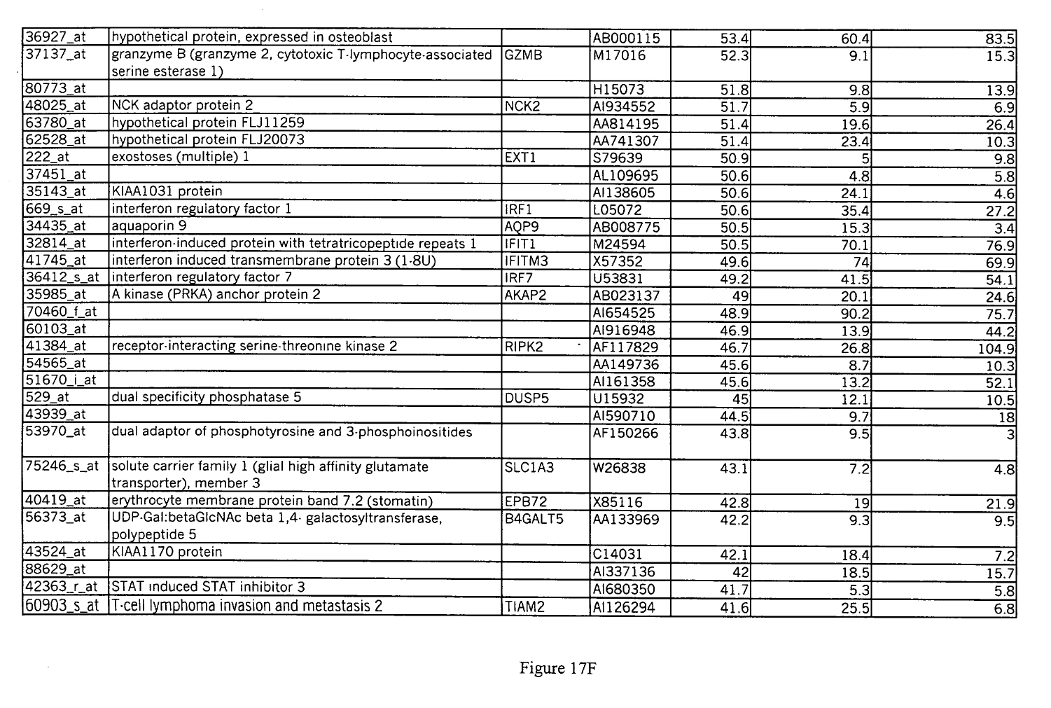

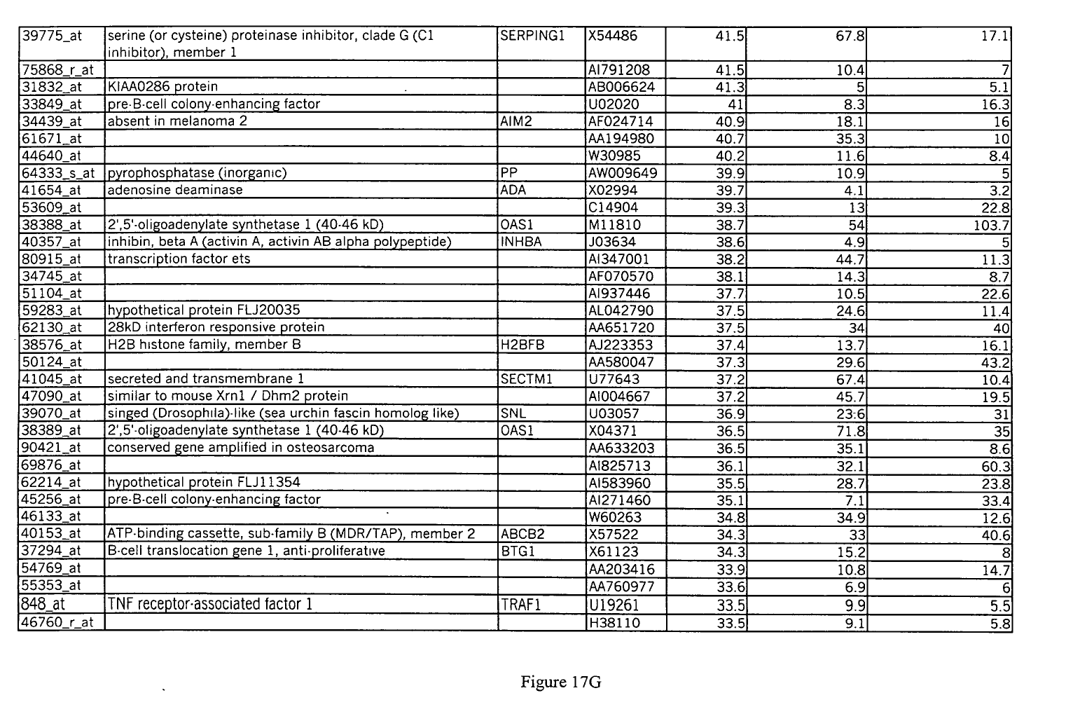

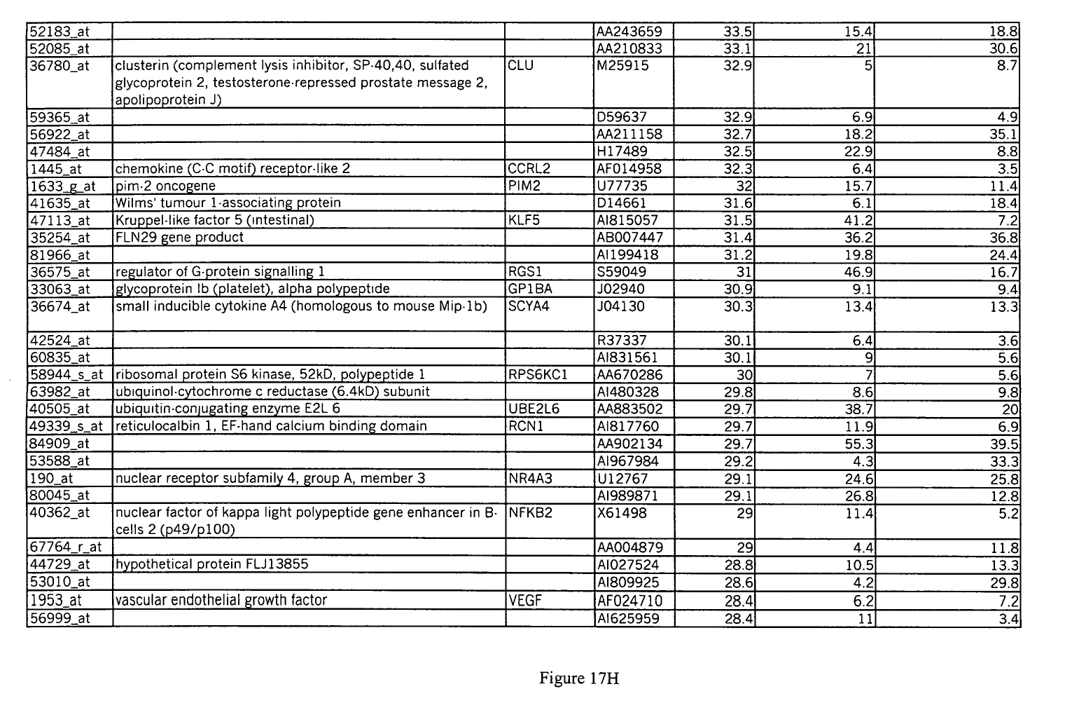

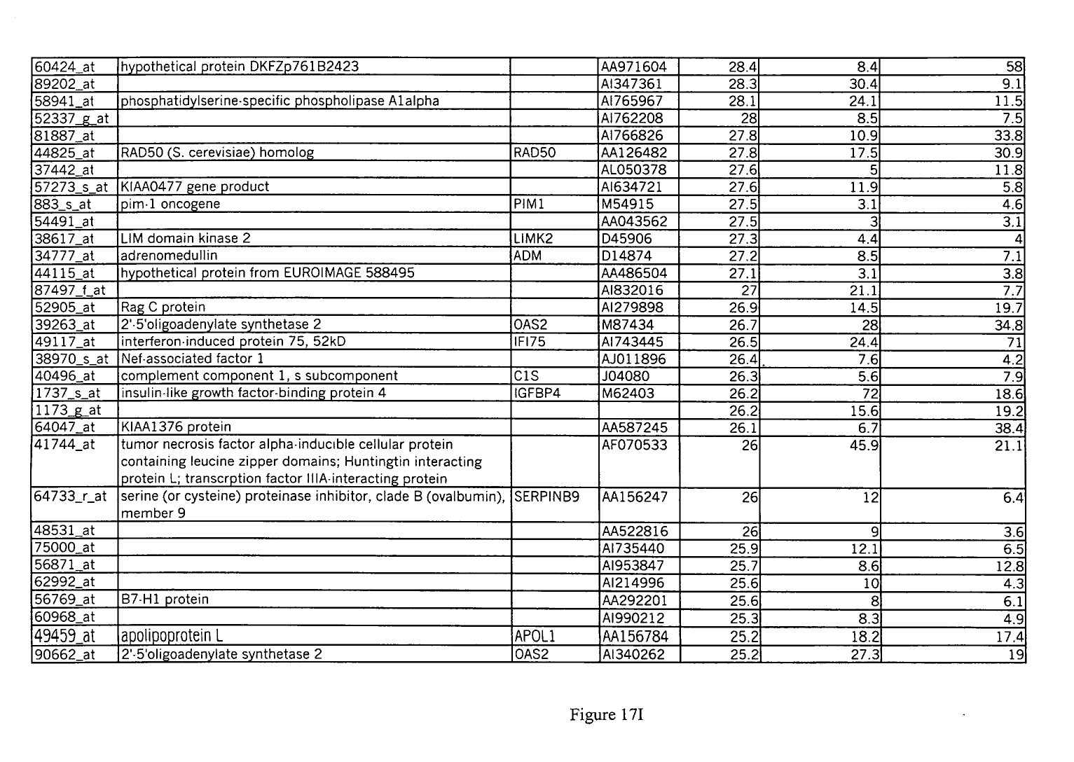

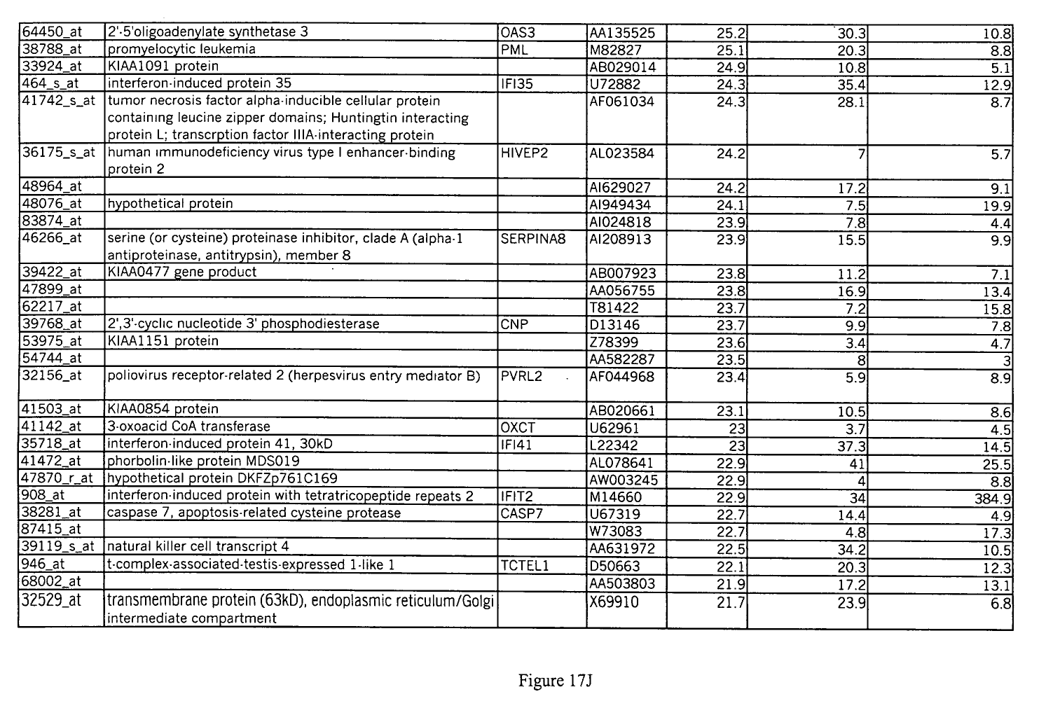

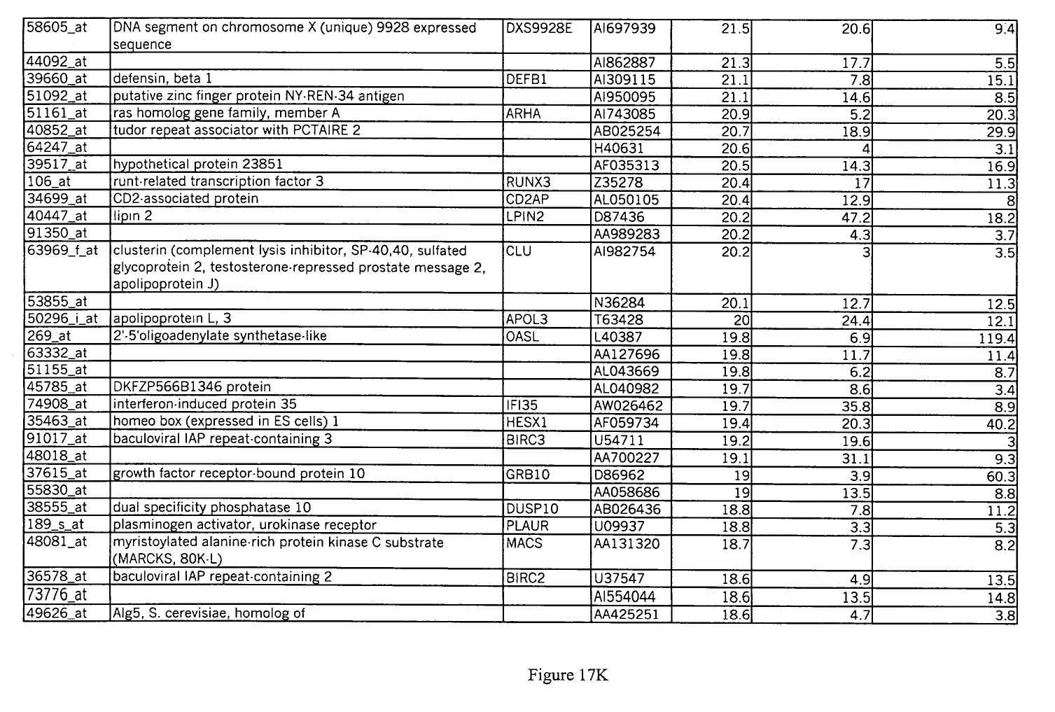

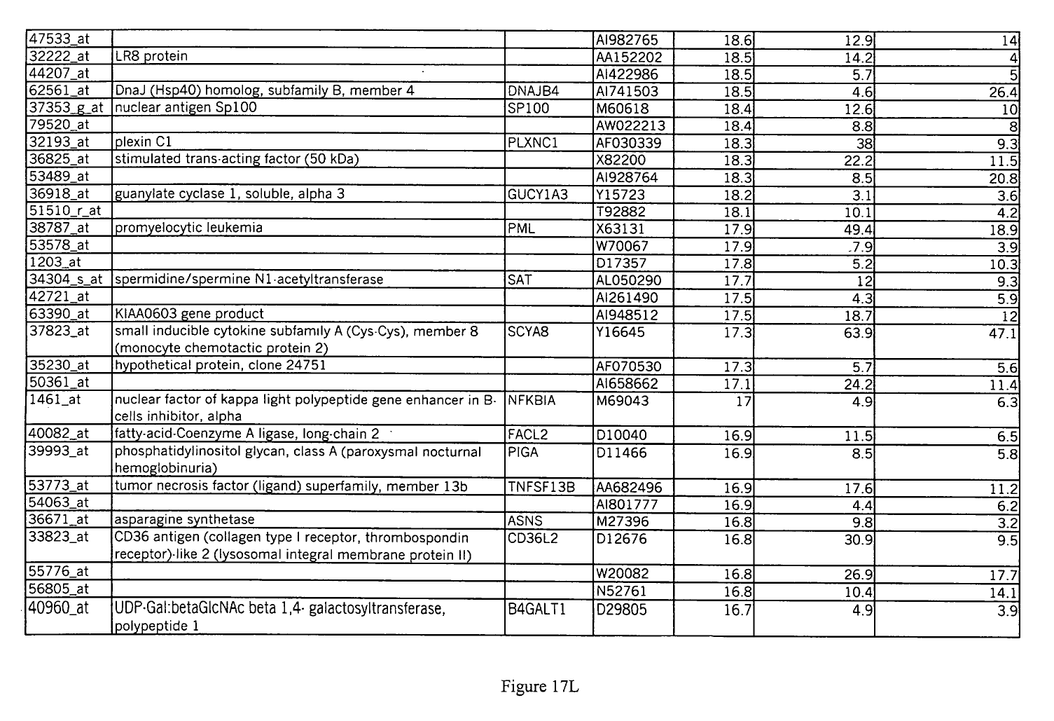

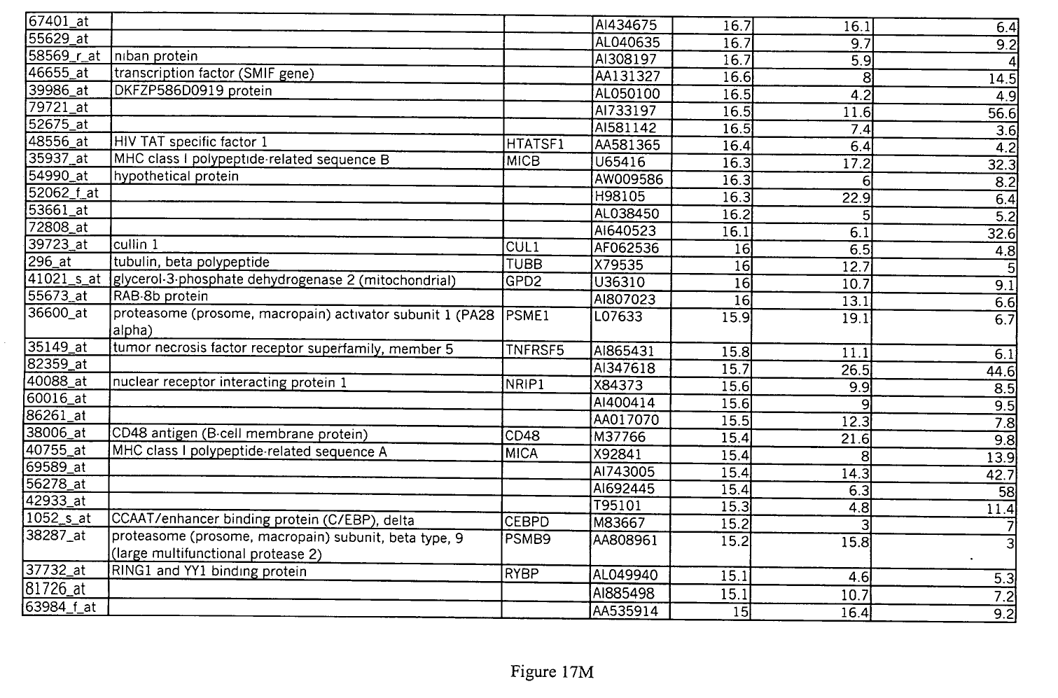

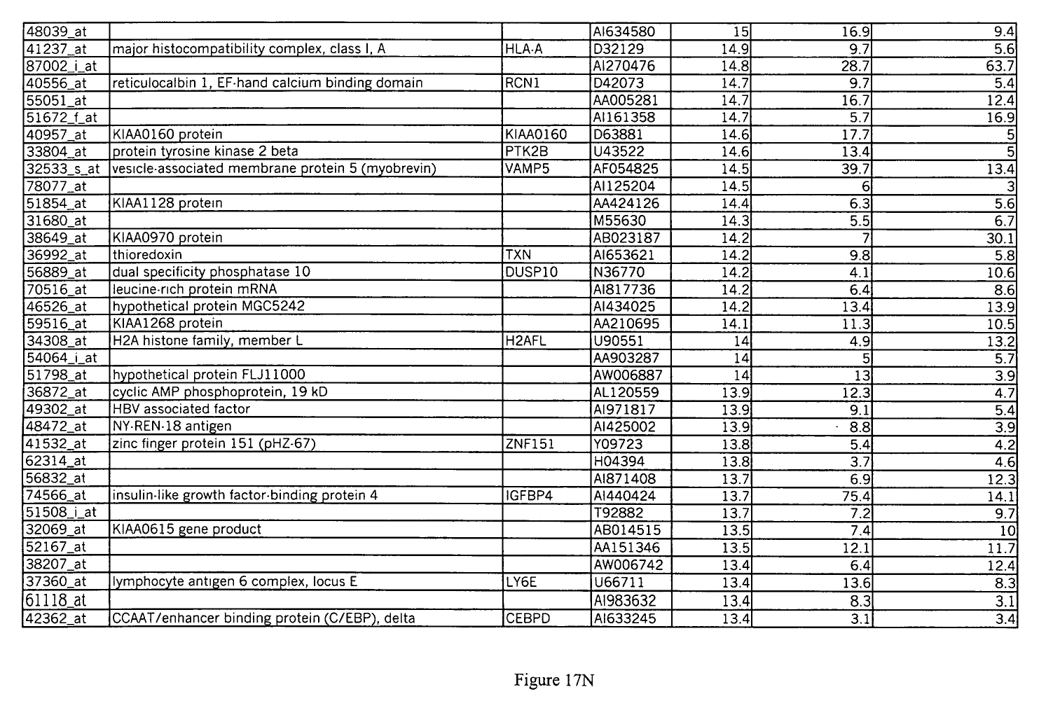

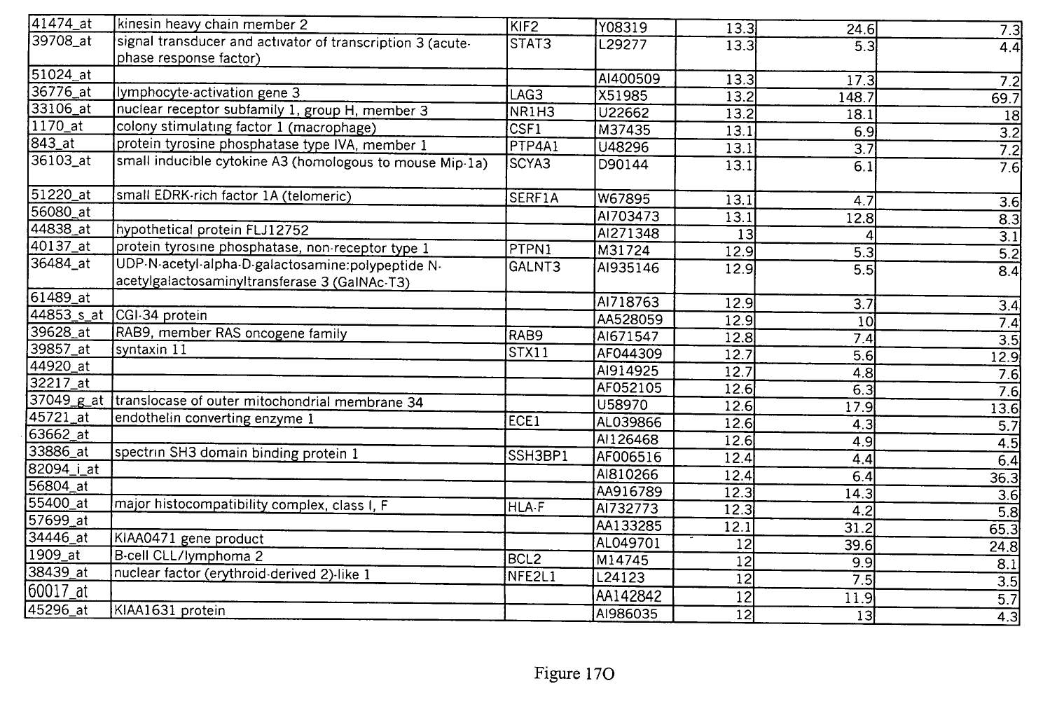

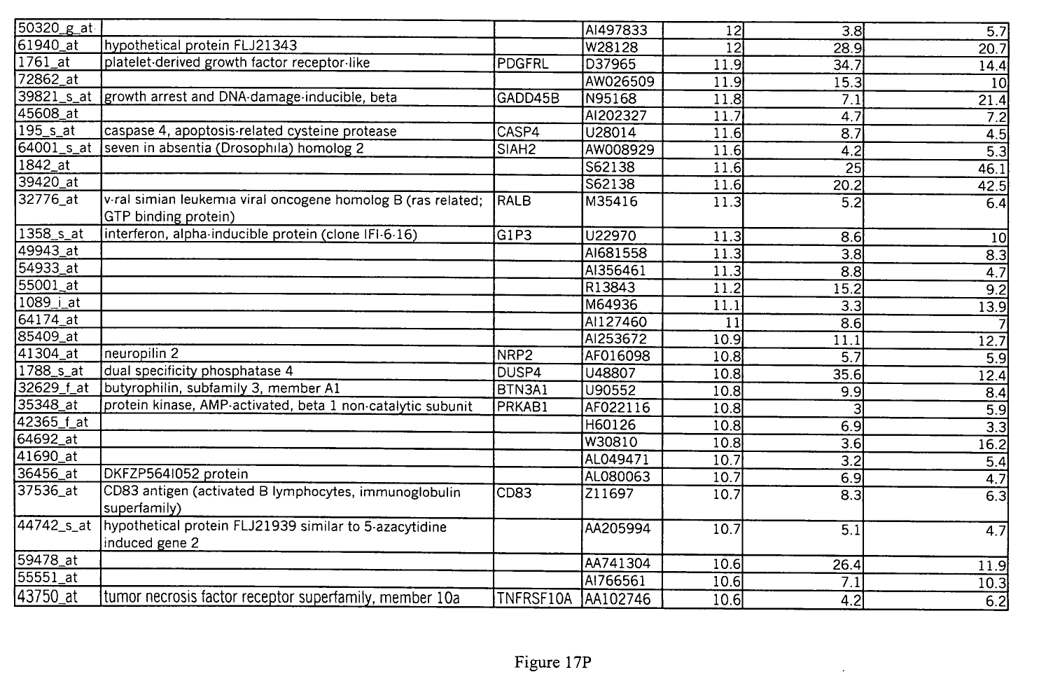

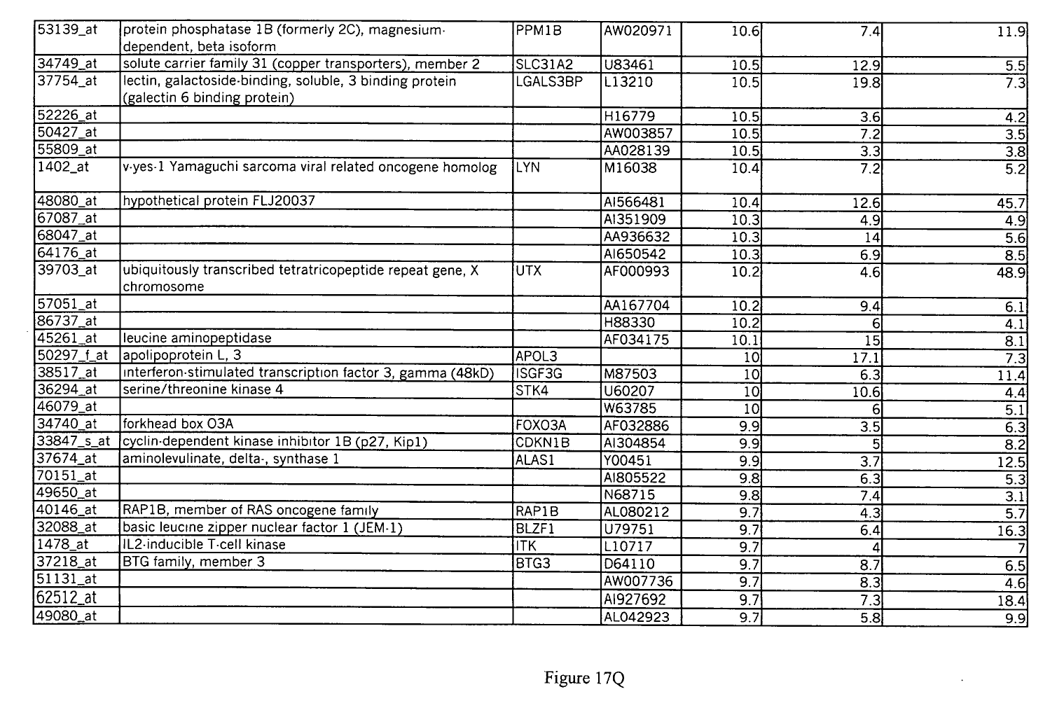

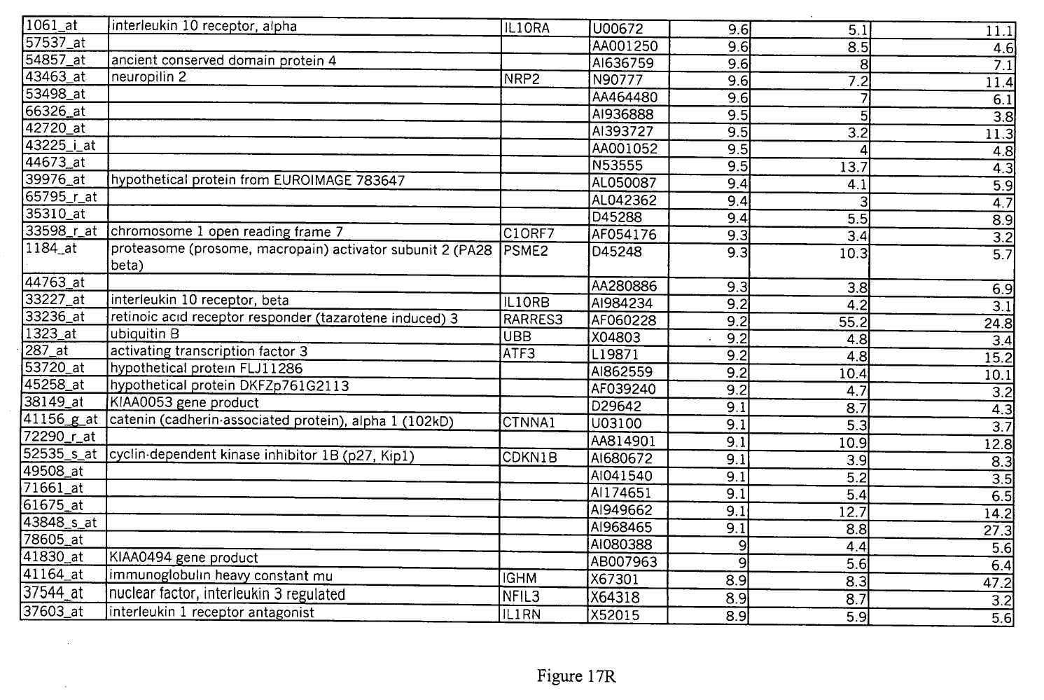

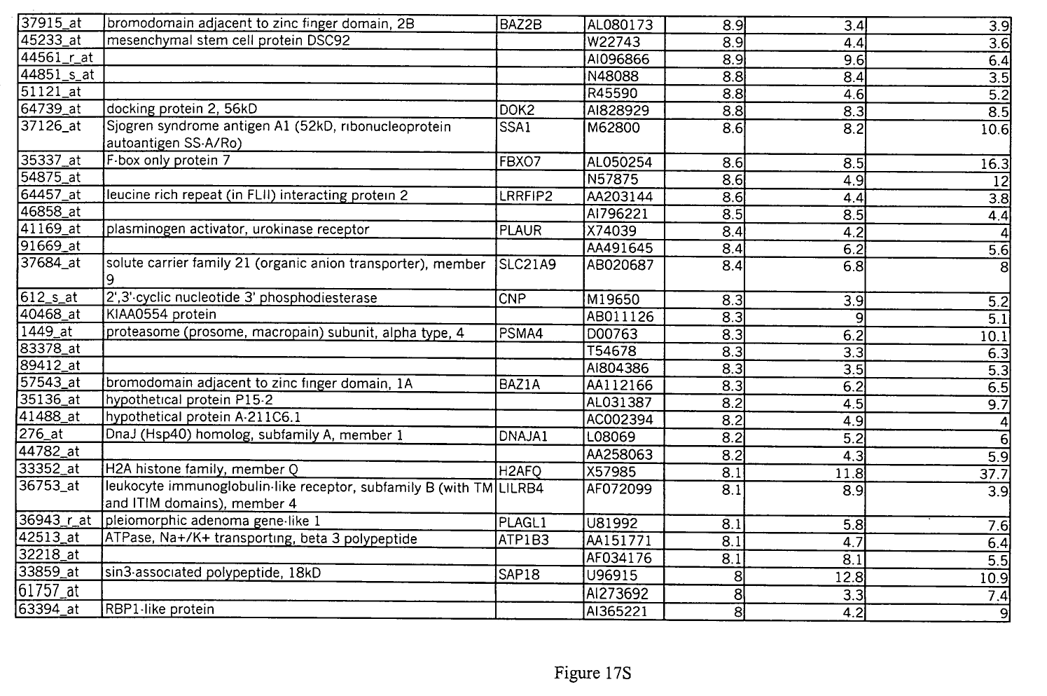

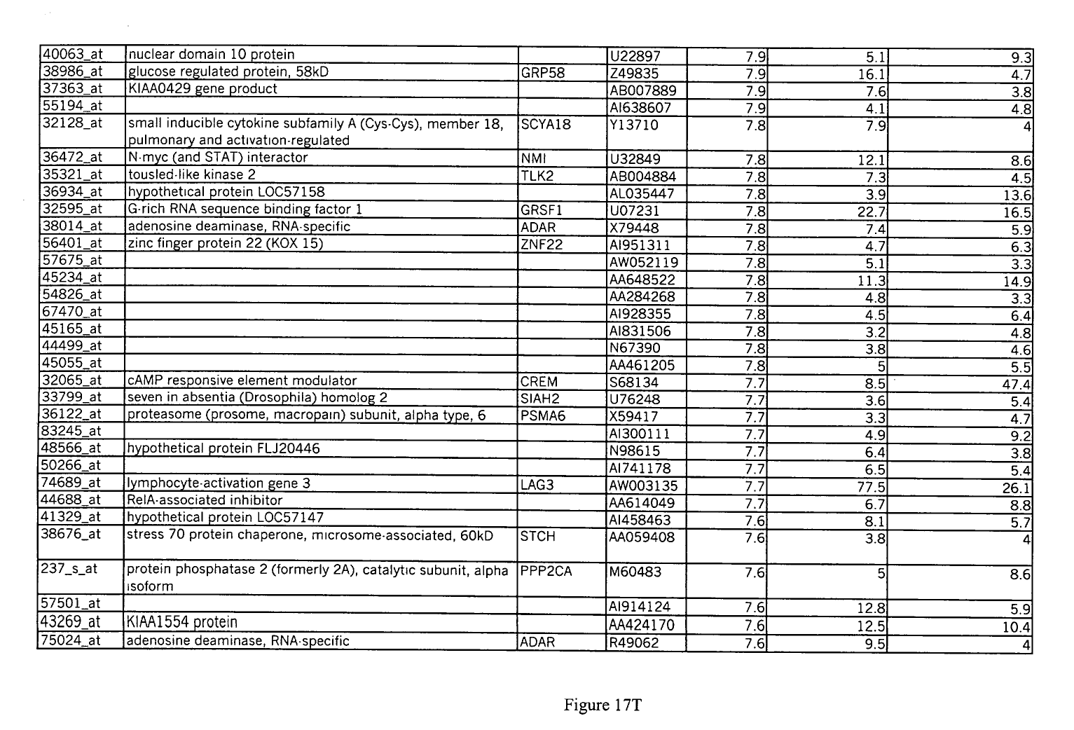

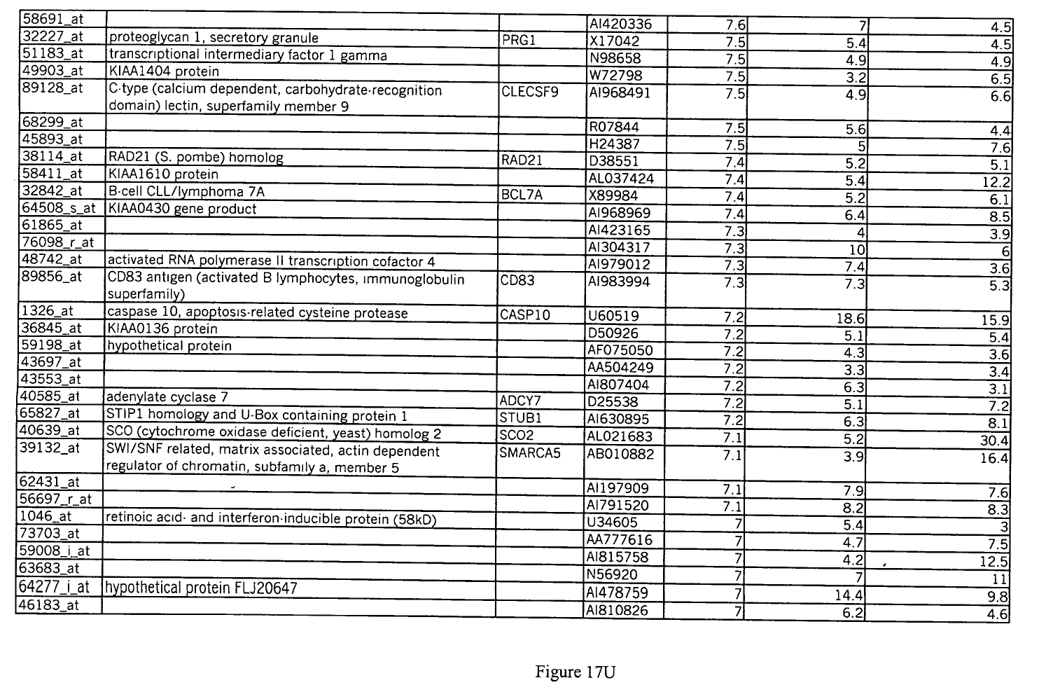

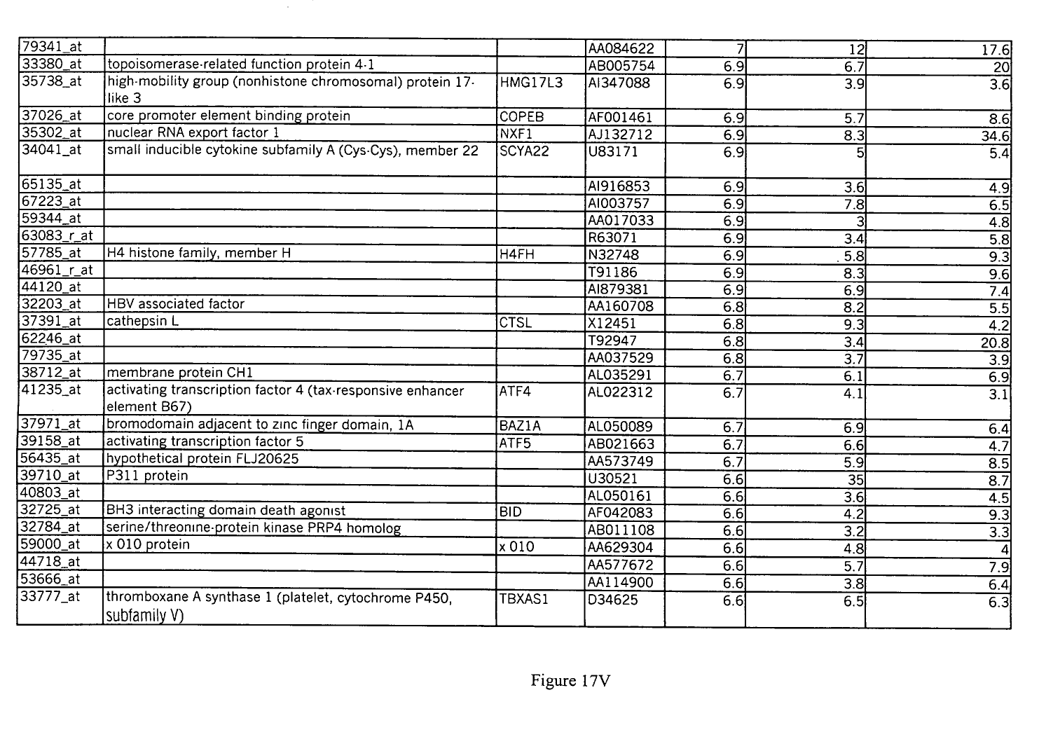

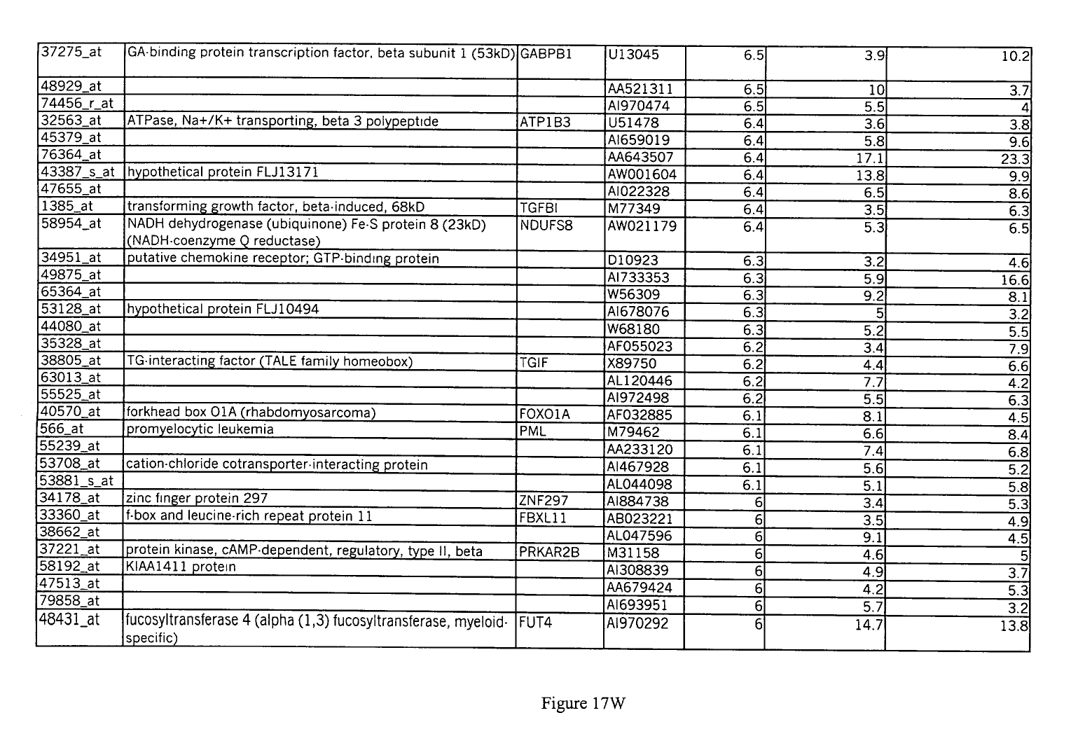

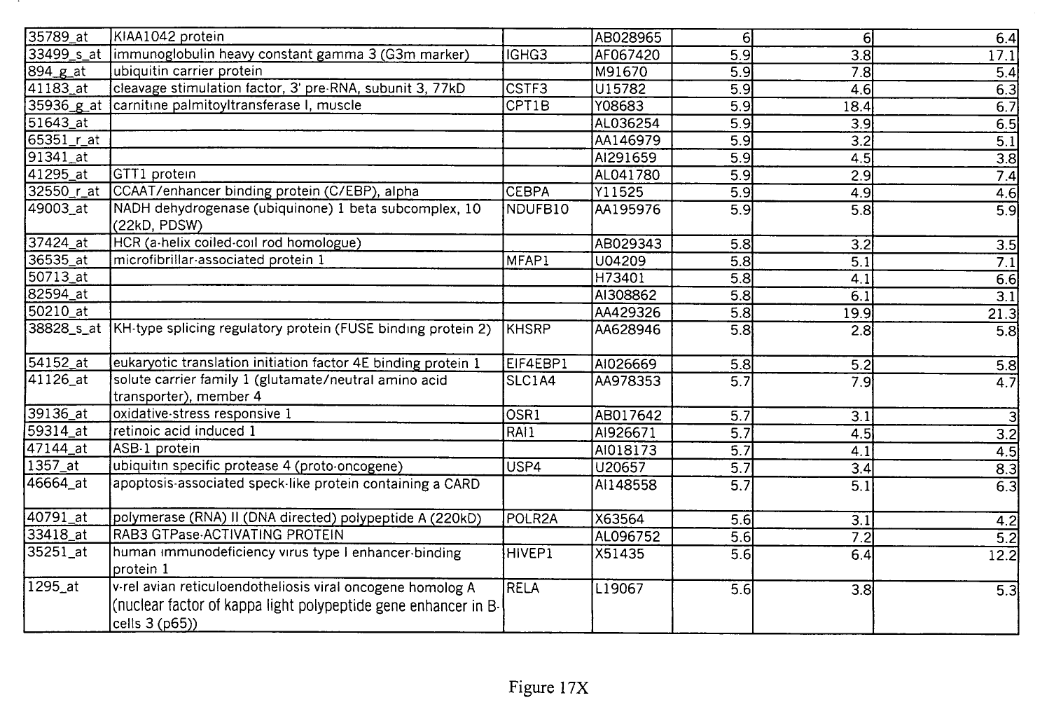

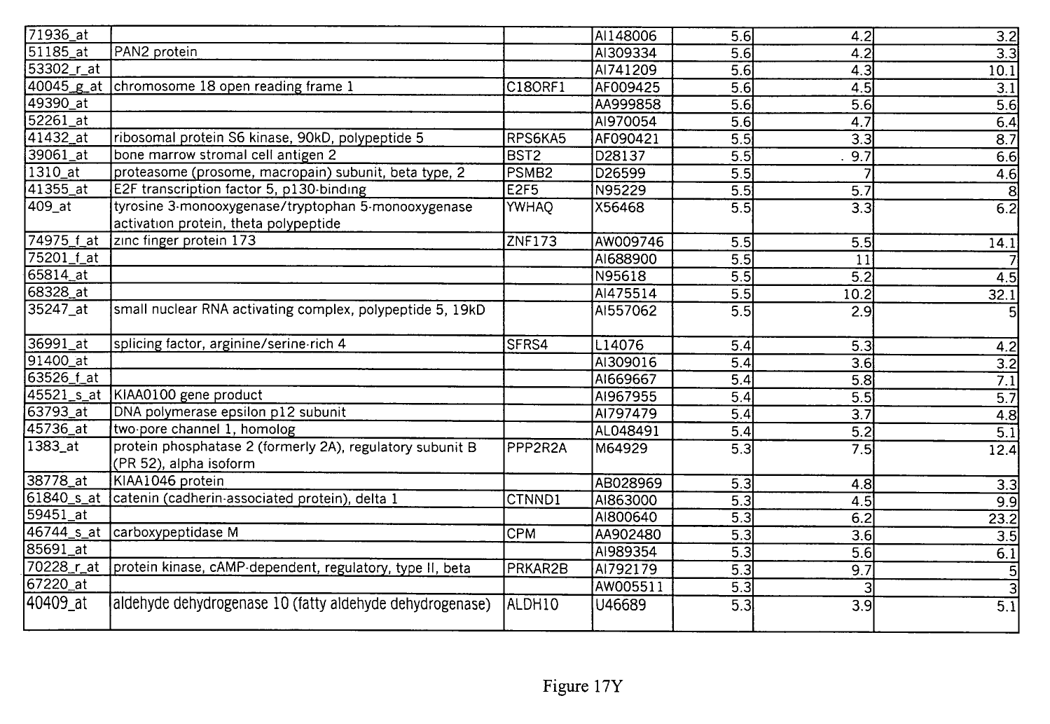

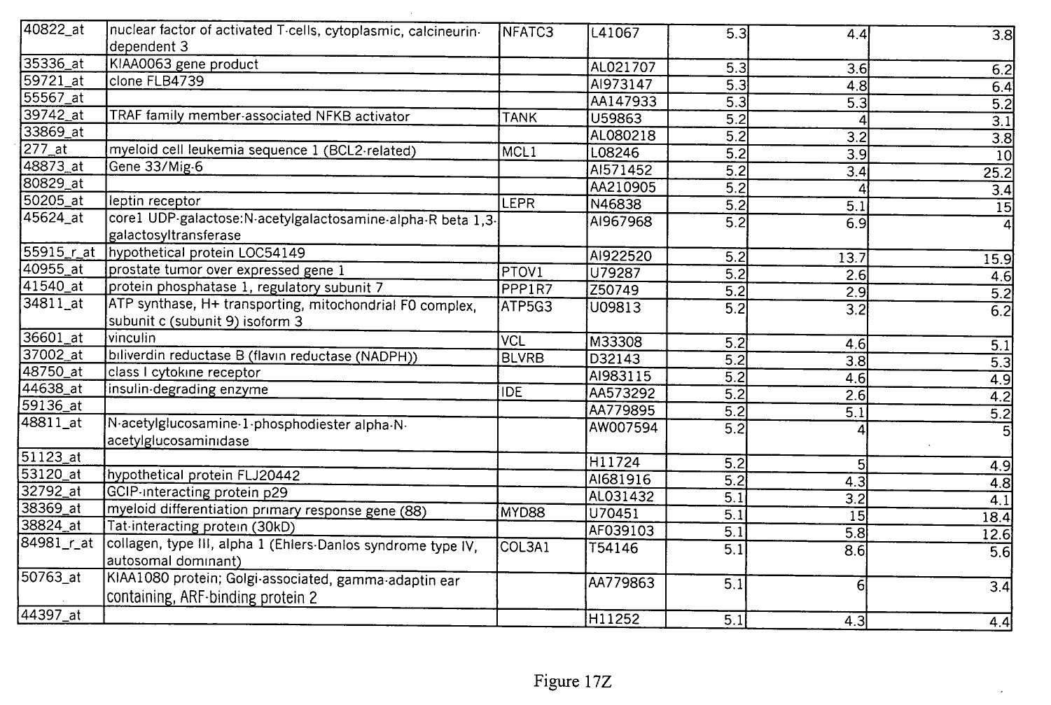

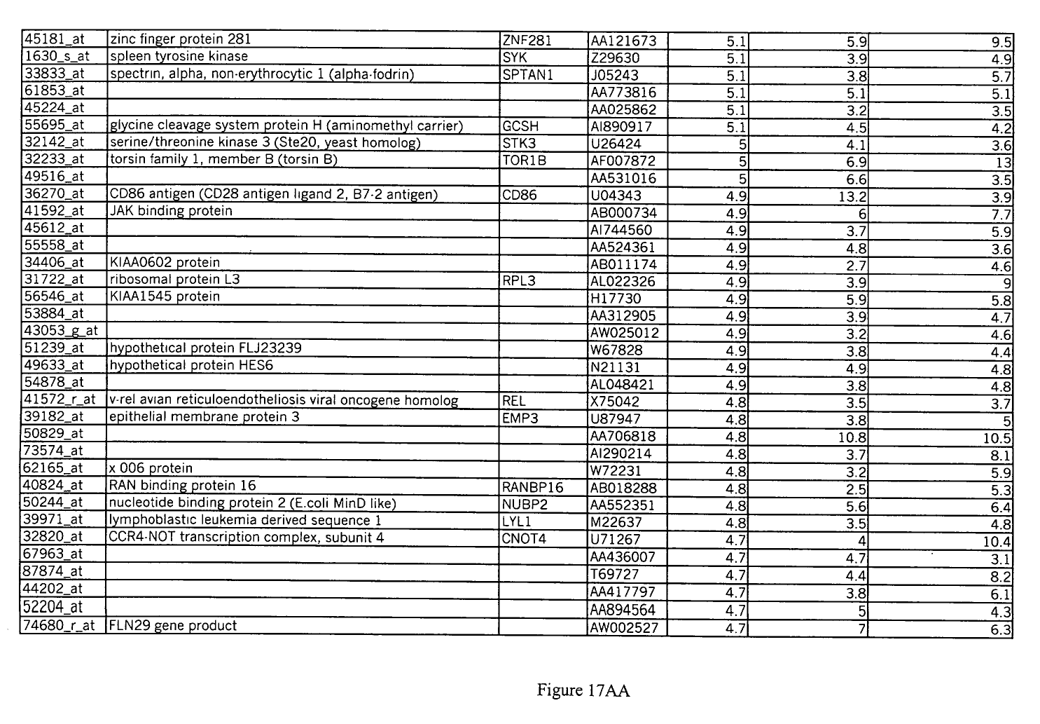

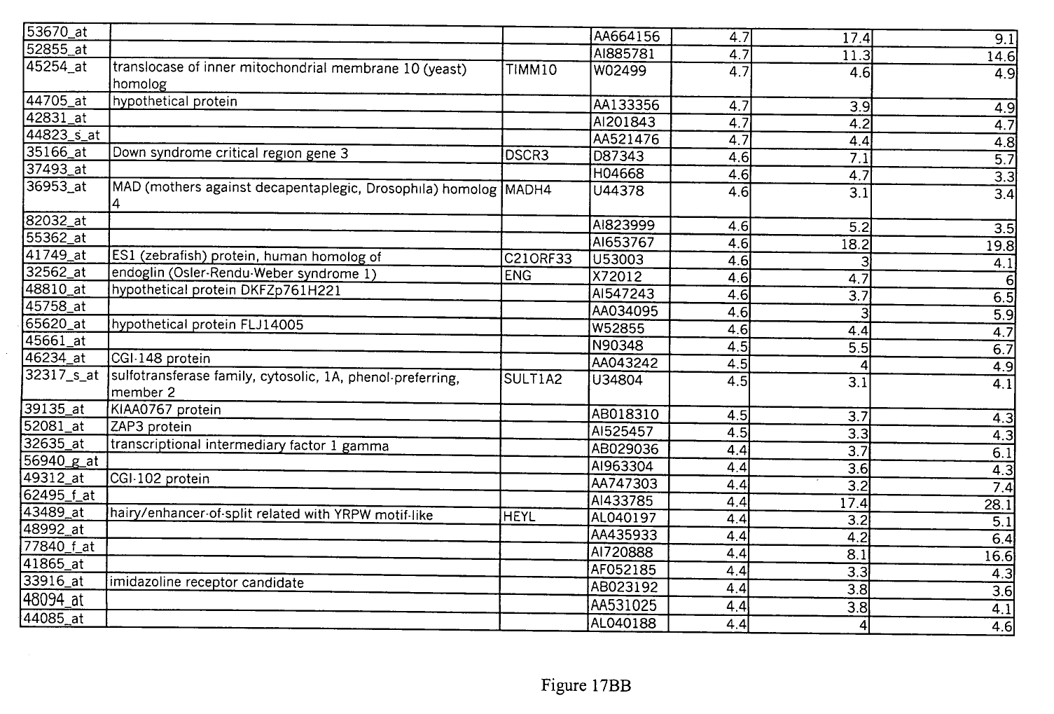

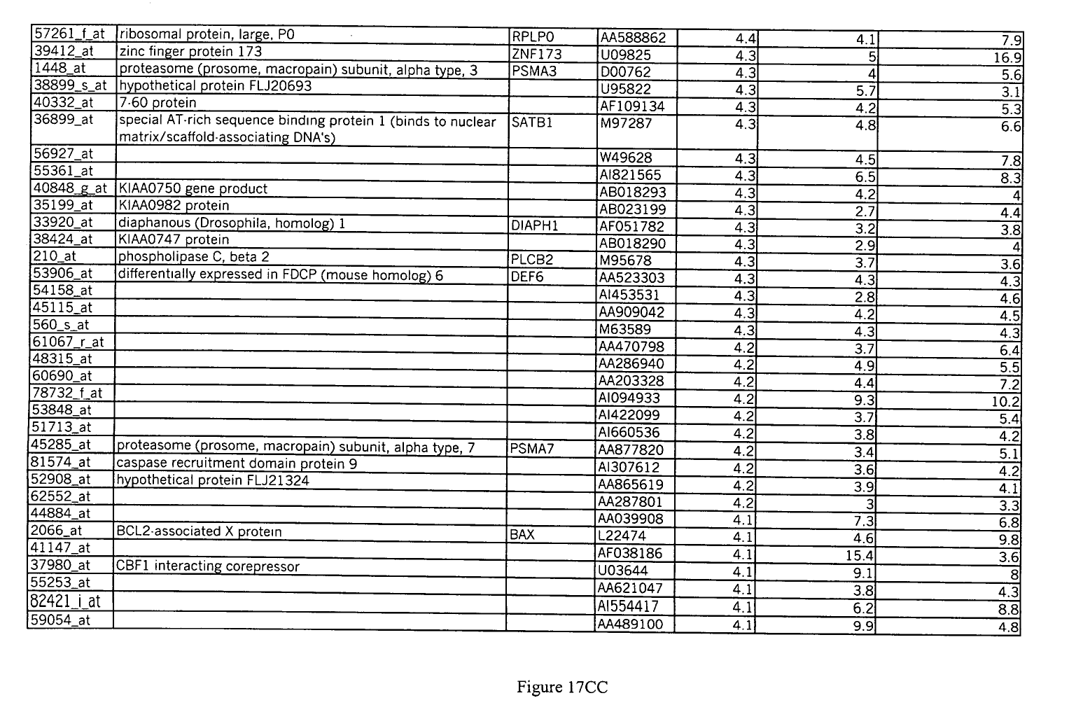

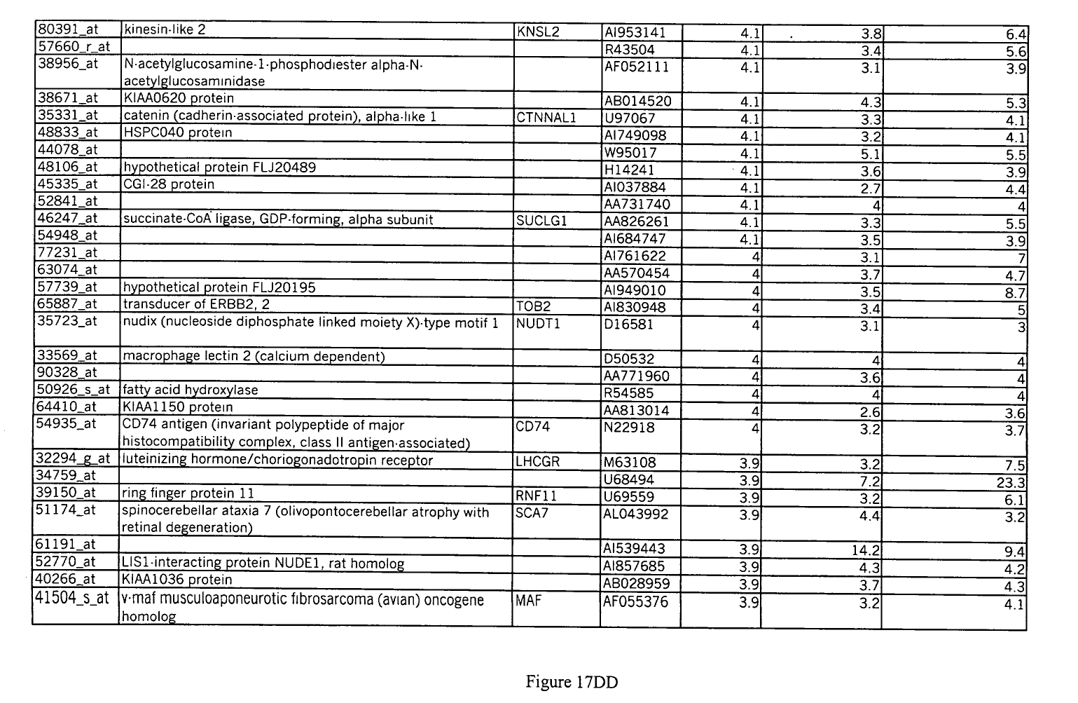

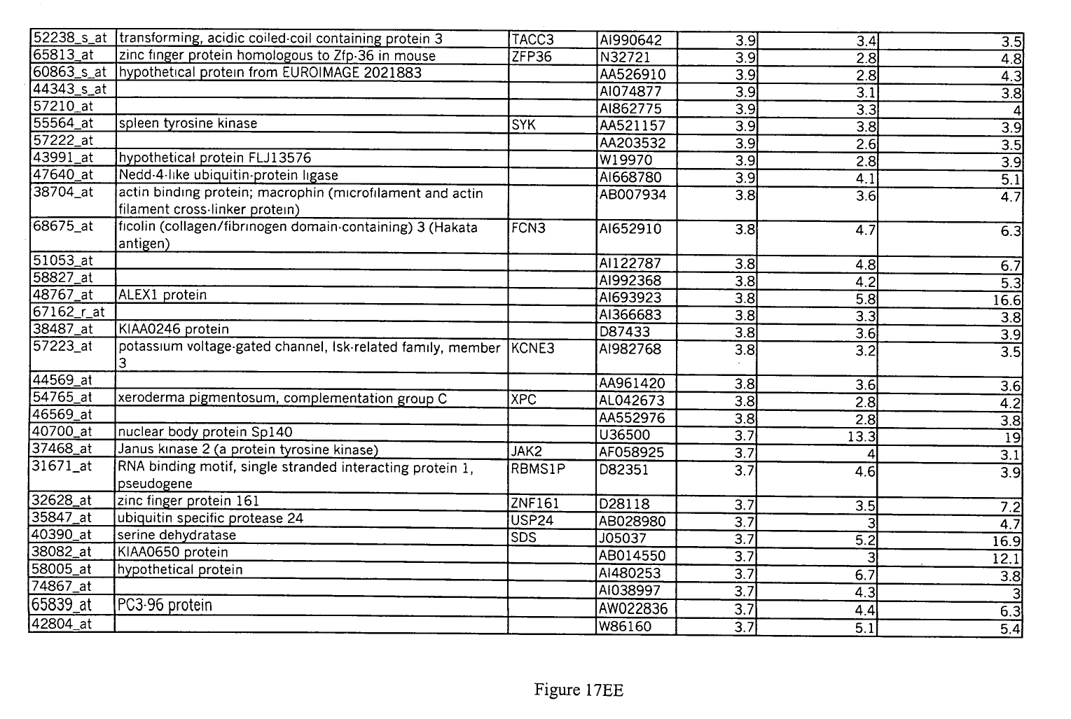

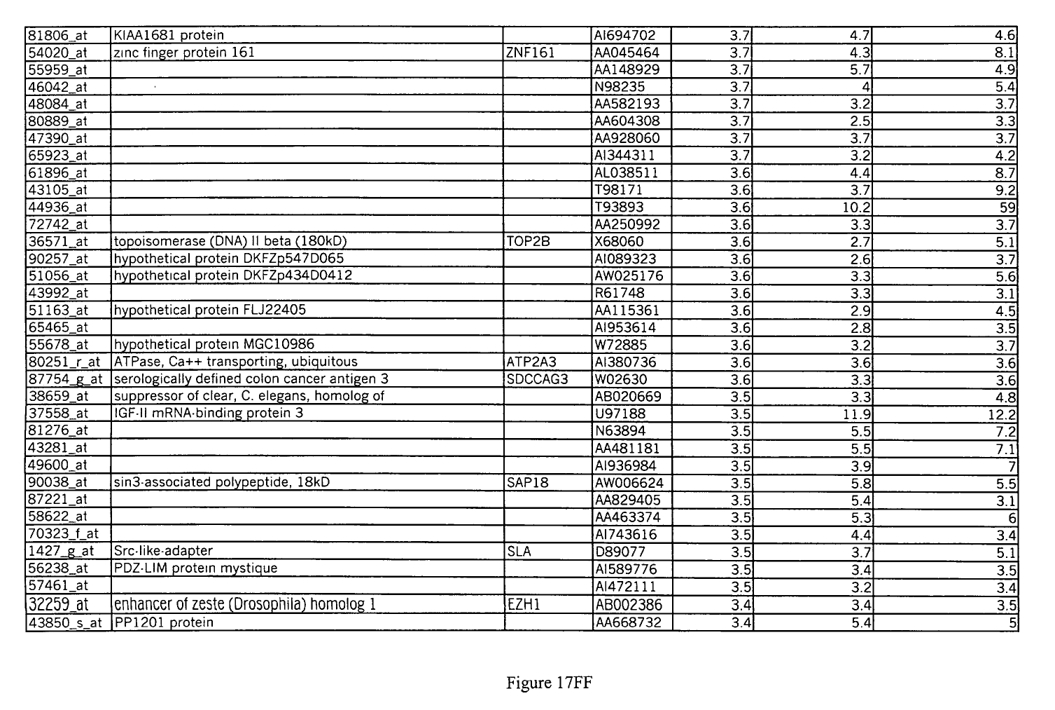

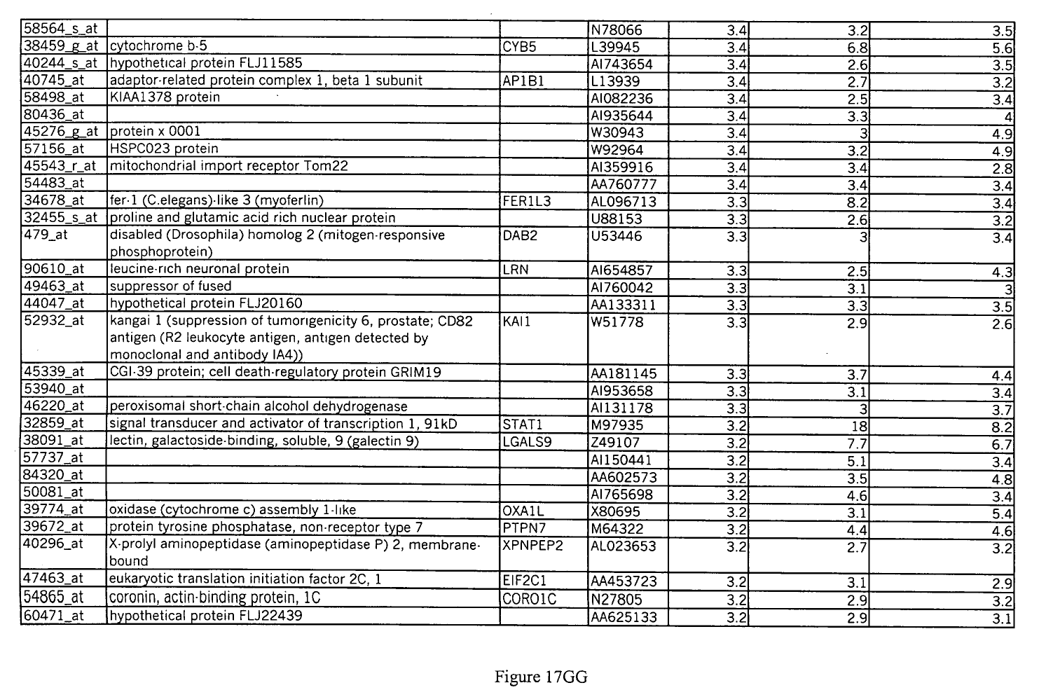

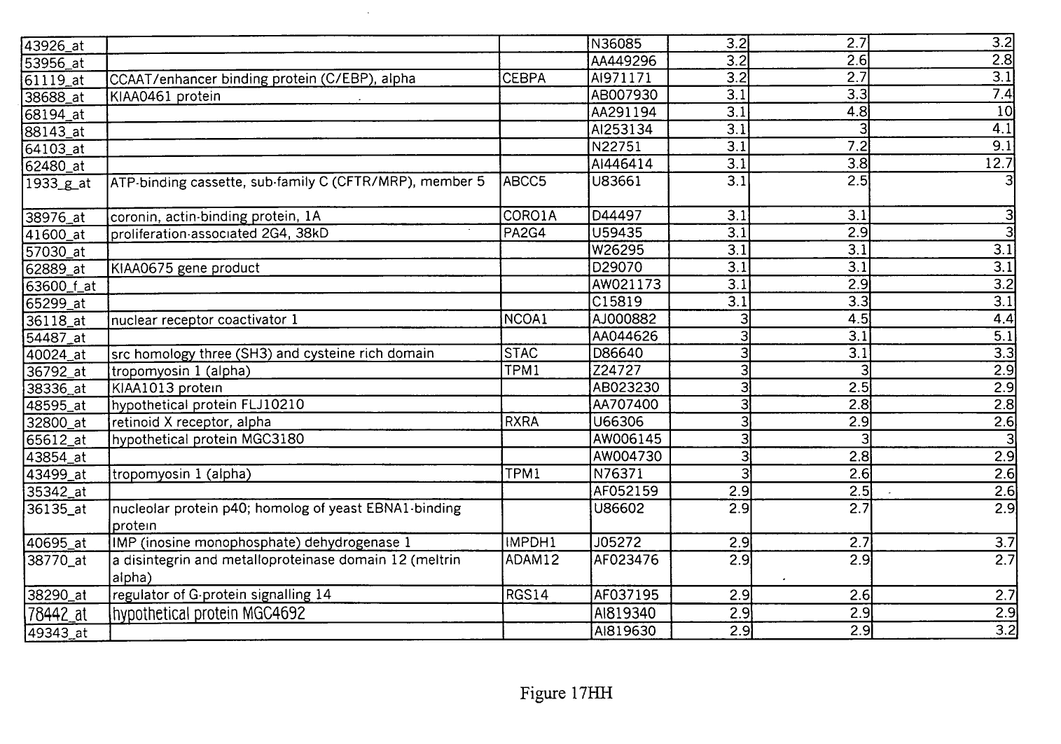

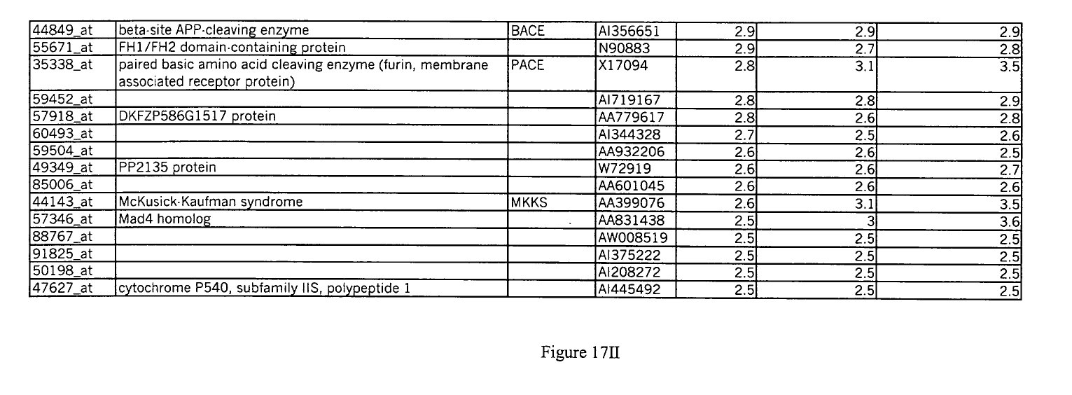

Additionally, the present invention provides stimulus-responsive (FIGS. 1A-1GG, 2A-2Z, 3A-3N, 11A-11AAAAAAA, 13A-13ZZZZZZ, and 15A-15KKKK), stimulus-specific (FIGS. 4A-4F, 5A-5C, 12A-12AA, 14A-14BB, 16A-16F) and common stimulus-responsive (FIGS. 6A-6E and 17A-17II) genes. Thus, the present invention provides information regarding the genes that are important in the selective immune response to a specific pathogen and in the common immune response elicited by all infecting organisms (FIG. 7A), thereby providing additional targets for diagnosis and therapy.

“Stimulus-responsive” genes, as used herein, refers to genes that are regulated in response to a pathogen or a component of a pathogen which elicits an immune response. “Stimulus-responsive” and “pathogen-regulated” can be used interchangeably. “Stimulus-specific” genes, as used herein, refers to genes that are specifically-regulated by a pathogen, pathogen-class or component thereof. “Stimulus-specific” and “pathogen-specific” can be used interchangeably. “Common stimulus-responsive genes”, as used herein, refers to genes that are regulated in response to two or more pathogens, pathogen classes, or components thereof. “Common stimulus-responsive” and “common regulated” genes can be used interchangeably.

As used herein, stimulus-responsive genes are determined (i.e., selected) by satisfying one of the following criteria: the ratio of gene expression score in response to a stimulus differs by greater than 1.2-fold compared to control media at two consecutive time points or greater than 4-fold for one timepoint for upregulated genes; or less than minus 1.4-fold for four consecutive timepoints for down-regulated genes. Common regulated genes are selected based on an intersection of the pathogen upregulated genes described above across all pathogens. Common down-regulated genes were selected based on a ratio less than −1.2-fold for three consecutive points across all pathogens.

Stimulus-specific genes are identified (i.e., selected) by satisfying the following criteria: the ratio of gene expression score in response to the pathogens differs by greater than 2.5-fold compared to control media for all three donors or greater than 1.4-fold at two consecutive time points for all three donors. It is clear that the present invention can be used to generate databases comprising stimulus-responsive genes, stimulus-specific genes, and/or common stimulus-responsive genes (see FIGS. 1A-1GG, 2A-2Z, 3A-3N, 4A-4F, 5A-5C, 6A-6I, 11A-11AAAAAAA, 12A-12AA, 13A-13ZZZZZZ, 14A-14BB, 15A-15KKKK, 16A-16F and 17A-17II). These databases will have many applications in medicine, research and industry. Specifically, the gene profiles described herein and identified by the methods of the present invention can be used to identify a pathogen or pathogen class to which a dendritic cell has been exposed.

In an alternate embodiment, the present invention is directed toward a method of identifying a pathogen comprising the steps of contacting one or more immature dendritic cells with a pathogen or with one or more immunogenic components thereof; isolating and labeling mRNA from said dendritic cells; detecting labeled mRNA from said dendritic cells such that a gene expression profile is produced; and analyzing the gene expression profile relative to one or more reference gene expression profiles such that at least one stimulus-specific gene is identified, thereby identifying a pathogen for which the stimulus-specific gene is specific. The present invention is useful in many clinical and non-clinical methods including but not limited to testing environmental, industrial and residential isolates for pathogens.

Diagnosis of infection is the first critical step in treatment of patients. In another embodiment, the present invention is directed toward a method of diagnosing infection in a mammal comprising the steps of isolating mRNA from one or more dendritic cells in a mammal; contacting said mRNA with at least one stimulus-responsive gene probe wherein hybridization of a stimulus-responsive gene probe to said mRNA is indicative of infection in said mammal. The ratio of gene expression of the stimulus-specific gene in comparison to a control, as described herein, identifies the pathogen (if present) responsible for the infection. Additionally, there are variations of the present invention which are well within the abilities of one of ordinary skill in the art. For example, to identify infection in a vertebrate, the gene probe can be a stimulus-specific or a common stimulus-responsive gene probe. The criteria for use of each probe type are described herein. Methods of determining hybridization of a probe to its target are well characterized in the art, for example, affinity chromatography, nucleic acid blotting, and microarrays. The amount or level of hybridization of the stimulus-responsive probe can be positive or negative, or a combination of both when measured in a time course. Additionally, the absence of a gene can be indicative of infection.

In another embodiment, the present invention is directed toward a method of diagnosing infection in a mammal comprising the steps of isolating proteins from one or more dendritic cells from said mammal; contacting said proteins with at least one stimulus-specific antibody wherein binding of a stimulus-specific antibody to said proteins are indicative of infection in said mammal. Methods of protein extracts from cells and preparing antibodies are well known in the art. Antibodies can be polyclonal and/or monoclonal. Mixtures of stimulus-specific antibodies can also be used to detect stimulus-specific proteins. Lack of binding of a stimulus-specific antibody to the extracted proteins can be diagnostic of infection by a pathogen(s) or type of pathogens(s).

In another embodiment, the present invention is directed toward a method of diagnosing infection by a specific pathogen in a mammal comprising the steps of isolating mRNA from one or more dendritic cells in a mammal; determining gene profile of at least one stimulus-specific gene wherein expression of a stimulus-specific gene is indicative of infection by a pathogen to which the stimulus-specific gene is specific. The profile of the stimulus-specific gene can be positive or negative. “Positive” as defined herein means an increase in gene expression profile relative to a control gene profile. “Negative” as defined herein means a decrease in the gene expression profile relative to a control profile.

In another embodiment, the present invention is directed toward a method of predicting prognosis for an infected individual by analyzing gene expression profiles of stimulus-responsive genes, wherein a specific expression profile is correlated with a clinical outcome. For example, the prognosis of a patient whose gene profile as determined by the present invention correlates with a poor prognosis. The advantage of the present invention would be to utilize a more aggressive treatment for the patient. Correlation is performed for a population of individuals who have been tested for gene profiles of stimulus-specific or common stimulus-responsive genes as described herein. To perform such analysis, the gene profile for at least one stimulus-specific or common stimulus-responsive gene is determined for a set of the individuals, some of whom exhibit a particular trait, and some of which exhibit lack of the trait. Correlation can be performed by standard statistical methods such as a Chi-squared test and statistically significant correlations between gene profile(s) and phenotypic characteristics are noted. For example, it might be found that the presence of Candida-specific gene profile correlates with eczema.

Such correlations can be exploited in several ways. In the case of a strong correlation between a gene-profile and a disease for which treatment is available, detection of a stimulus-responsive gene profile in a human or animal patient may justify immediate administration of treatment, or at least the institution of regular monitoring of the patient. In the case of a weaker, but still statistically significant correlation between a stimulus-specific gene profile and human disease, immediate therapeutic intervention or monitoring may not be justified. Nevertheless, the patient can be motivated to begin simple life-style changes (e.g., diet, exercise) that can be accomplished at little cost to the patient but confer potential benefits in reducing the risk of conditions to which the patient may have increased susceptibility by virtue of a stimulus-responsive gene profile. Identification of a stimulus-responsive gene profile in a patient correlated with enhanced receptiveness to one of several treatment regimes for a disease indicates that this treatment regime should be followed.

Alternatively, the present invention could be used to formulate a therapeutic regimen. As used herein “therapeutic regimen” is defined as treatment of a patient with pharmacological agents. In one embodiment, the present invention is directed to a method of formulating a therapeutic regimen comprising the steps of identifying the pathogen, and formulating a therapeutic regimen accordingly. Additionally, repeated assessment of a patient for a pathogen can determine the effectiveness of a therapeutic regimen which can then be altered accordingly. Methods of determining the effectiveness of a therapeutic regimen is known in the art.

Vaccine development is one of the most important prospective health safeguards in medicine. The present invention is ideally suited for vaccine optimization. In one embodiment, the present invention is directed to a method of vaccine optimization comprising the steps of contacting dendritic cells with a test vaccine, isolating mRNA from said dendritic cells; determining gene expression profiles in said dendritic cells; and selecting a test vaccine which elicits the gene expression profile which is indicative of an optimized vaccine. Construction of vaccines and generation of variant test vaccines are known to those of skill in the art. As used herein “variant test vaccine” is a different composition of the original test vaccine. The optimized immune response refers to the immune response which promotes the highest level of pathogen immunity or killing. Methods of measuring immunity and killing of pathogens is well characterized in the art. The optimized vaccine is determined as described herein and defined as the test vaccine which elicits the strongest immune response. For example, the optimized vaccine should at least have the response produced by the standard vaccine for the pathogen, if available. The gene expression profile of the optimized vaccine may differ from other non-related vaccines.

Since dendritic cells initiate the innate and adaptive immune response, they are well suited to triggering an immune response to a specific antigen by the present invention. In one embodiment, the present invention is directed to an ex vivo therapeutic treatment for a pathogen comprising the steps of contacting a patient's dendritic cells with a pathogen or immunogenic components thereof such that said dendritic cells become activated; returning activated dendritic cells to the patient such that the activated dendritic cells trigger an immune response against said pathogen. Activated dendritic cells express specific cell-surface markers including CD86, CD83, and HLA-DR. Methods of screening for markers is known to those of skill in the art.

In another embodiment, the present invention is directed to an ex vivo therapeutic treatment of tumors comprising the steps of contacting a patient's dendritic cells with tumor cells or immunogenic components thereof ex vivo such that said dendritic cells become activated; and returning activated dendritic cells to the patient such that the activated dendritic cells trigger an immune response against said tumor cells or components thereof. Any type of tumor cells may be used.

Dendritic cells are also involved in antigen tolerance which is important in autoimmune diseases (such as, for example, arthritis) and in organ transplantation. In another embodiment, the present invention is directed to an ex vivo therapeutic treatment of autoimmunity comprising the steps of contacting a patient's dendritic cells with self-antigens or immunogenic components thereof ex vivo such that said dendritic cells become activated; returning activated dendritic cells to the patient such that the activated dendritic cells do not trigger an immune response against said self-antigens or components thereof.

The most common problem associated with organ transplantation is graft-rejection. Graft-rejection is the process by which the transplanted tissue is recognized as non-self and triggers an immune response which leads to the destruction of the transplanted tissue. In another embodiment, the present invention is directed to an ex vivo therapeutic treatment of graft-rejection comprising the steps of contacting a patient's dendritic cells with graft-tissue or components thereof such that said dendritic cells become activated; returning activated dendritic cells to the patient such that activated dendritic cells do not trigger an immune response against said graft-tissue or components thereof.

In another embodiment, the present invention is directed to a method of measuring the immune response to a stimulus comprising the steps of contacting dendritic cells with a stimulus; isolating mRNA from said dendritic cells which determining a gene profile such that at least one stimulus-responsive gene is identified which is indicative of an immune response. The gene profile of one or more genes can be measured as described herein and by methods known to one of skill in the art and include, but are not limited to, affinity chromatography, nucleic acid blotting and microarrays.

In another embodiment, the present invention is directed to a method of measuring the immune response to a stimulus comprising the steps of contacting dendritic cells with a stimulus; isolating and labeling mRNA from said dendritic cells; contacting a DNA microarray with labeled mRNA from said dendritic cells; and measuring the gene profile relative to control stimulus such that at least one stimulus-responsive gene is identified. In another embodiment, the present invention is directed toward measuring the gene profile of dendritic cells in response to a stimulus comprising the steps of contacting dendritic cells with a stimulus; isolating and labeling RNA from said dendritic cells; contacting a DNA microarray with labeled mRNA from said dendritic cells; and measuring the gene profile relative to control stimulus such that at least one stimulus-responsive gene is identified. The preferred source of dendritic cells is peripheral blood, although they may be isolated from any source including other body fluids, tissues, organs or obtained from a commercially available source. The methods of dendritic cell isolation and culture are well known to those of skill in the art and described herein. As used herein “immature” dendritic cells are defined as dendritic cells which have not been exposed to an antigen. Methods for production of immature dendritic cells are described herein.

Stimulus (e.g., pathogen) suitable for use in the present invention are bacteria, yeast, fungi, and viruses including Escherichia coli, Staphylococcus aurens, influenza virus, Candida albicans, lipopolysaccharide (LPS), polyI:C, yeast mannan and ds RNA (double-stranded RNA) or components thereof. Bacteria can be gram positive or gram negative. Viruses can be RNA (single-stranded or double-stranded) or DNA viruses. Bacteria, yeast, and viruses should be used at a concentration sufficient to ensure that the majority of dendritic cells are contacted with a microbe such that every cell responds (but still low enough that the microbe does not overgrow in the tissue culture plate and/or kill the dendritic cells). Bacteria and fungi can be used at a multiplicity of infection (MOI) from about 1 to 10,000. In particular embodiments, the bacterial or fungi can be used at MOI from about 1.5 to about 1000; about 2 to about 50; about 3 to about 20; or about 5 to about 10 MOI. Additionally, the stimulus can be physical, chemical, or electrical. Furthermore, the stimulus can comprise inorganic chemicals, organic chemicals or a combination thereof. The stimulus preferably elicits an immune response when exposed to dendritic cells. Methods of measuring an immune response are well characterized in the art.

In one embodiment of the present invention, dendritic cells are cultured with a stimulus. Subsequently, mRNA is isolated from dendritic cells at various time points after exposure. mRNA isolation from cells in culture is art standard (see Sambrook et al., Molecular Cloning: A Laboratory Manual, Cold Spring Harbor Laboratory Press; Ausubel, F. M., et al., Current Protocols in Molecular Biology, Greene Publishing Assoc. and Wiley-Interscience 1987, & Supp. 49, 2000, the teachings of which are incorporated herein by reference). mRNA is then labeled by standard methods (see above references). mRNA may be labeled with any appropriate molecule, for example fluorophores, biotin, radioactive nucleotide, and dye. In a preferred embodiment, mRNA is fluorescently labeled. In one embodiment, labeled mRNA can then be hybridized to DNA microarrays. The DNA microarray can be any high-density oligonucleotide microarray, for example, GeneChip® HU 6800 (Affymetrix, Santa Clara, Calif.). Hybridization of labeled mRNA to the DNA microarray is known to those of skill in the art (see Tamayo et al. Proceedings of the National Academy of Science (1999) 96:2907-2912; Eisen et al. Methods Enzymology (1999) 303:179-205).

Quantitation of gene profiles from the hybridization of labeled mRNA/DNA microarray is done by scanning the microarrays to measure the amount of hybridization at each position on the microarray with an Affymetrix scanner (Affymetrix, Santa Clara, Calif.). For each stimulus a time series of mRNA levels (C={C1, C2, C3, . . . Cn}) and a corresponding time series of mRNA levels (M={M1, M2, M3, . . . Mn}) in control medium in the same experiment as the stimulus is obtained. Quantitative data is then analyzed. Ci and Mi are defined as relative steady-state mRNA levels, where i refers to the ith timepoint and n to the total number of timepoints of the entire timecourse. μM and σM are defined as the mean and standard deviation of the control time course, respectively.

Alternatively, labeled RNA can be hybridized to a filter or other solid support containing target nucleic acids which comprise the pathogen-specific or common regulated genes described herein. Hybridization conditions should be stringent enough to ensure specific binding between labeled RNA and target genes. Stringent hybridization conditions are known to one of ordinary skill (see Sambrook et al., Molecular Cloning: A Laboratory Manual, Cold Spring Harbor Laboratory Press; Ausubel, F. M., et al., Current Protocols in Molecular Biology, Greene Publishing Assoc. and Wiley-Interscience 1987, & Supp. 49, 2000). Quantitation of specific hybridization can be performed by any suitable method including scintilation and densitometry.

The generation of the stimulus-specific databases by the present invention will provide a method for identifying a pathogen in a patient. Since dendritic cells are found in peripheral blood, they are easily obtained by venous puncture. In one embodiment, the present invention is directed to a method of identifying a pathogen comprising the steps of isolating a patient's dendritic cells, isolating and labeling mRNA from said dendritic cells, contacting a DNA microarray with labeled mRNA from dendritic cells, and measuring and analyzing the gene profile such that at least one stimulus-specific gene is identified. The stimulus-specific gene is then used to search the stimulus-specific database such that a pathogen is identified. The stimulus-specific gene may be specific for more than one pathogen. Also, the pathogen can belong to a family of pathogens.

The present invention encompasses kits for all of the disclosed embodiments. The present invention provides a list of genes regulated in response to E. coli, Candida albicans, influenza virus and components thereof. The methods of the present invention can be used to identify pathogens and diagnose infections. Additionally, the present invention provides methods for optimizing vaccines and treating autoimmune diseases such as arthritis and graft-rejection.

While this invention has been particularly shown and described with references to preferred embodiments thereof, it will be understood by those skilled in the art that various changes in form and details may be made therein without departing from the scope of the invention encompassed by the appended claims. The teachings of all references cited herein are incorporated herein by reference.

### EXAMPLE 1

Preparation of Dendritic Cells

Elutriated human monocytes (Advanced Biotechnologies Inc., Columbia Md.) were grown in RPMI with 10% fetal bovine serum (Life Technologies, Rockville, Md.) supplemented with 1000 U/ml GM-CSF (R&D Systems) and 1000 U/ml IL-4 (R&D Systems) for 7 days in 24 well plates (106 cells in 1 ml medium/well), and fed every 2 days after plating (1).

Stimulation of Dendritic Cells

On day 7, dendritic cells (DC) (described above) were harvested and aliquoted into 100 mm plates at 1×10⁷ cells/plate and incubated at 37° for 60 min. Pathogens or their components were then added to DC cultures at amounts shown below. Stimulated cells showed mature DC dendrite formation, were less adherent at 24 hours, expressed typical DC markers (cd83, cd86), and stimulated allogeneic T cell proliferation at DC:T ratios of 1:1000-1:10. The following micro-organisms were used at the titers shown: E. coli SD54 (ATCC) (5:1 MOI) (kindly provided by J. Nau), Influenza A/PR/8/34 (750 HAU/ml (Hemagglutinin units/ml); the amount of virus needed to infect 50% of the cells was approximately 5-10 HAU/ml, gift of J. Hermann), Candida albicans HLC54 (5:1 MOI) (gift of G. Fink); and the following components at the concentrations shown: LPS (lipopolysaccharide) from E. coli 055:B5 (1 mg/ml, Sigma L-2880), polyI:C (25 mg/ml, Pharmacia; endotoxin levels were <0.2 EU/ml, which are not sufficient to induce TNFα expression in DC), mannan from S. cerevisiae (1 mg/ml, Sigma, M 7504). One concern is that the observed pathogen specific responses are due to different titers of each pathogen, rather than to intrinsic differences in pathogen recognition. This is unlikely because many genes within the common response are induced to equal levels by all stimuli, suggesting that some pathways can be engaged equally by all pathogens; the titers used were shown to saturate expression of known maturation markers; all the pathogens were phagocytosed by DC; and in some cases, we repeated microarray measurements with lower titers and concentrations and found similar gene expression profiles (data not shown).

Summary of Donors with Pathogens and Components

The following table is a summary of the stimuli added to DC from each donor.

| DONOR | STIMULI |
|---|---|
| Donor D | polyI:C |
| Donor F | E. coli, influenza, LPS, polyI:C |
| Donor H | E. coli, influenza, LPS |
| Donor I | C. albicans |
| Donor M | C. albicans, mannan |
| Donor O | mannan |
| Donor T | E. coli, influenza, C. albicans |

RNA Preparation and Microarray Hybridization

At each timepoint (0, 1 hr, 2 hr, 4 hr, 8 hr, 12 hr, 18 hr, 24 hr, 36 hr), 1×10⁷ cells were harvested and lysed using Trizol (Molecular Research Center). Total RNA was isolated, labeled and prepared for hybridization to HuGeneFL oligonucleotide arrays (Affymetrix, Santa Clara, Calif.) using standard methods (Golub, et al., Science 286:531-537 (1999)).

Hybridization was carried out overnight with 15 mg of labeled RNA product, and arrays were scanned on Affymetrix scanners. Measurements were made at multiple timepoints after DC stimulation in order to allow comparison of response levels between six unrelated human donors with variable kinetics and to define different phases in DC activation. influenza and E. coli responses were always measured in the same donor.

Gene Expression Measurements

Gene expression measurements were stored, analyzed, using analysis described herein. mRNA expression kinetics and induction levels were validated with enzym-linked immunosorbent assay (ELISA) measurement of tumor necrosis factor (TNF) α, IL 12/p40, IL-10 and MCP-1 protein levels.

Statistical Analysis

Data Collection and Validation

1.7×10⁶ individual gene measurements were stored, analyzed and visualized using a set of database and analysis tools developed in the lab. Array measurements were normalized with a reference array hybridized with sample from the same donor, using the median of the hybridization signals of all genes with P-calls (P-calls according to the Affymetrix software) as a scaling factor. Data for several missing timepoints were interpolated using flanking timepoints. Gene expression profiles were validated by measuring the protein levels of four secreted factors (TNFα, IL12p40, IL10, MCP-1) using standard ELISA measurements which confirmed both the kinetics and the relative induction levels in response to each stimulus.

Scoring System

A time series of mRNA fluorescence levels, R={R₁, R₂, R₃, . . . , Rₙ} were collected in dendritic cells (DC) exposed to each pathogen or compound, and a control series of mRNA levels, C={C₁, C₂, C₃, . . . , Cₙ}, in untreated DC from the same donor. Rᵢ and Cᵢ are steady-state mRNA hybridization measurements (“average difference” in Affymetrix terminology) at the ith timepoint; n is the total number of timepoints. A score, Sᵢ=(Rᵢ−μc)/σc, was devised to measure significant deviation of the stimulated expression level at one timepoint, Rᵢ, from the mean μc of the control timecourse. By using σc, which is the standard deviation of the control timecourse, the score penalizes genes with high noise in the media control, thus allowing extraction of the most robust data.

Pathogen-Regulated Gene

Upregulated genes were selected according to the following criteria applied at the same time to all three donors: (i) Sᵢ>1.2 for ≧two consecutive timepoints or (ii) Sᵢ>4 for ≧one timepoint. Downregulated genes were selected if Sᵢ<−1.4 for ≧four consecutive timepoints; however, since the signal-to-noise was lower than for upregulated genes, the filter was relaxed further and the most robust down-regulated genes that passed the filter for 2 out of 3 donors and had the same trend in the third donor were selected.

Common Regulated Genes

Common upregulated genes were selected based on an intersection of the pathogen upregulated genes defined above (i and ii). Common downregulated genes were selected based on Sᵢ<−1.2 for ≧three consecutive timepoints across all pathogens.

Stimulus-Specific Genes

Fold expression level in response to a stimulus, A, and relative to control is defined as F_{A}=max{L₁, L₂, . . . , Lₙ₋₁} where L₁=geomean{R₁, R_{I+1}}/geomean{Cᵢ, Cᵢ₊₁}. The ratio of fold expression levels for a gene between stimulus A and B is defined as RF_{AB}=F_{A}/F_{B}. Genes that were regulated more strongly by one stimulus (A) than another stimulus (B) were identified in one of two ways: (i) average RF_{AB}=2.5 in three donors; or (ii) in three donors, Sᵢ>1.4 for ≧two consecutive timepoints for stimulus A (selects for a gene profile that is significantly different from control timecourse) and Sᵢ=1.2 for all points for stimulus B (gene profile closely matches the control timecourse). The final set of stimulus-specific genes (for example, FIG. 7A, stippled circles) were used to describe how the DC response differs biologically for each stimulus. The remaining genes that were regulated in stimulus A but not in stimulus B and did not pass the stimulus-specific filters above were not used in a comparative analysis since they are on the border between the groups of common and stimulus-specific genes.

All filters were used as described above except for:

(1) two-way comparisons (between DC responses to stimulus A vs. stimulus B) when the responses to A and B were not measured in the same donor for all replicates:

(i) Component-Pathogen Comparisons.

All component responses were measured in two donors while all pathogen responses were measured in three donors. To increase the stringency of comparison between component and pathogen, we required that the average RF_{AB}=2.5 (as for all other comparisons) and that F_{A}/F_{B}=2 for one experiment in which the component and pathogen are measured in the same batch of DC.

(ii) C. albicans-Pathogen Comparisons.

Since the same batch of DC were exposed only in one experiment (donor T) to all the pathogens (C. albicans, E. coli and influenza), the stringency of any pathogen comparison to C. albicans was increased by requiring that the average RF_{AB}=2.5 and that F_{A}/F_{B}=2 for the experiment in which all the pathogen responses were measured (donor T).

(2) regulated genes for component responses, which were measured in two donors, require a more stringent filter: upregulated genes were selected according to the following criteria for one experiment: (i) Sᵢ>2 for ≧two consecutive timepoints or (ii) Sᵢ>1.2 for ≧three consecutive timepoints or (iii) Sᵢ>5 for ≧one timepoint; the same criteria were applied to an independent experiment from a second donor, except (i) was replaced with a less stringent filter, Sᵢ>1.4 for ≧two consecutive timepoints, to take into account variations in level of expression between donors. Downregulated genes were selected if Sᵢ<−1.4 for ≧two consecutive timepoints.

Temporal Clustering

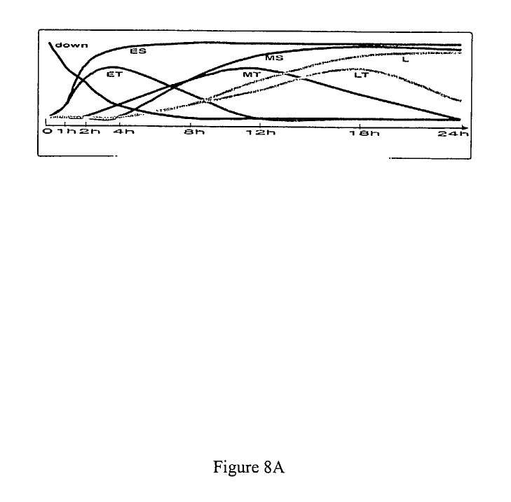

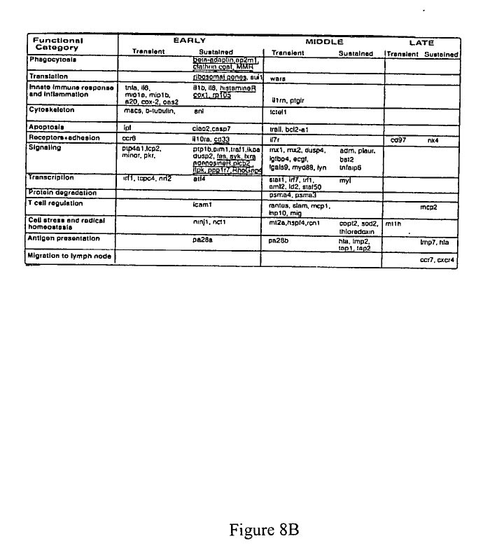

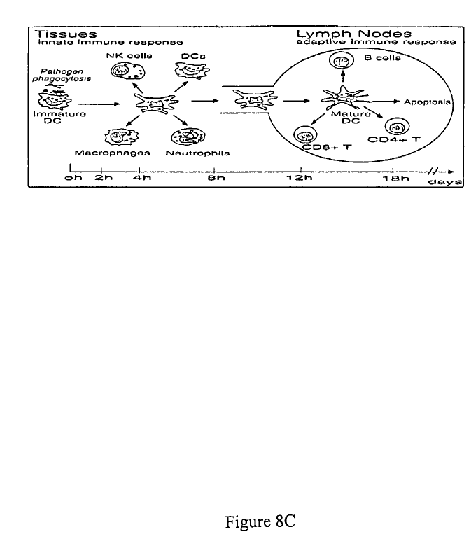

A self-organizing map algorithm (Tamayo, et al., Proc Natl Acad Sci U S A 96:2907-12 (1999)) was used to cluster genes together based on the similarity of their temporal expression profiles. This procedure was used to aid in classifying genes into six basic groups: three major groups which consist of genes that are expressed in an early, middle or late phase of the timecourse; and for each of these groups, the genes are further divided up into those that are expressed transiently or in a sustained fashion (FIG. 8A).

Functional Categories

Each of the regulated genes was also assigned into functional categories (e.g. chemokines, glycolysis, etc.) according to the public databases. The assignment of functional groups was verified and refined by comparison to the Proteome annotated database of genes, Human PSD™ (kindly provided through a collaboration with Proteome, Inc).

Neutrophil Migration

Neutrophil isolation and migration assays were done with standard techniques (J. E. Coligan et al., Eds., Current Protocols in Immunology (John Wiley & Sonds, 1999)). Directed neutrophil migration was measured using 96-well chambers with filters (Neuroprobe, Gaithersburg, Md.). 20,000 neutrophils were fluorescently labelled with PKH26 (Sigma, St. Louis, Mo.) and placed on top of the filter; DC supernatants were placed below (1:10 dilution). After incubating for 30′ at 37° C. and 10′ at 4° C., neutrophils were counted on the bottom of each well.

### EXAMPLE 2

### Identification of Pathogen Regulated Genes Using Affymetrix GeneChip® HuGenF1 Chip (6800 Genes) (3 Donors)

Oligonucleotide microarrays were used to test what extent DC discriminate between phylogenetically diverse pathogens and whether the commonly studied components of these pathogens are sufficient to account for the live pathogen response.

Human monocyte derived dendritic cells (DC) were exposed to a diverse set of organisms and compounds: a Gram-negative bacterial species, E. coli, and its cell wall component, lipopolysaccharide (LPS); a fungus, C. albicans, and yeast cell wall-derived mannan; and an RNA virus, influenza A, and double-stranded RNA (dsRNA). DC were cultured with pathogens or their components between 1 and 36 hours and RNA was isolated, labeled, and hybridized to microarrays (as described herein). Each pathogen stimulation was repeated in three independent donors, and each component stimulation was repeated in two donors. Genes with expression levels that changed in response to stimuli (termed regulated genes) were selected on the basis of significant and repeated differences in the expression levels of the treated and untreated samples across multiple time points. Of the approximately 6800 genes represented on the oligonucleotide array, a total of 1330 genes changed their expression significantly upon encounter with one of the pathogens or components. Such a large-scale change in gene expression demonstrated that DC are able to undergo a marked transformation in their cellular phenotype.

Analysis of the individual responses to pathogens showed that a unique number of genes was regulated by each pathogen. influenza and E. coli were able to modulate the expression of exclusive subsets of genes (7A and 7C), whereas C. albicans only modulated the expression of a subset of E.coli-regulated genes (FIG. 7B). In addition, gene expression was most rapidly induced by E. coli, less rapidly by C. albicans, and most slowly by influenza.

The intersection of the three different pathogen responses revealed a common set of 165 highly regulated genes (FIGS. 7A and 7D). To describe the dynamics of DC response after exposure to any of the three pathogens, we classified these genes according to their kinetics of expression and known biological functions (FIGS. 8A and 8B). Immediately after contact with any of the three pathogens, a rapid decline was observed in the transcript levels of genes associated with phagocytosis and pathogen recognition (FIG. 8B). At the same time, there was a transient increase in the expression of immune cytokines, chemokines, and receptors that contribute to the recruitment of monocytes, DC, and macrophages to the site of infection. Also strongly induced was a set of cytoskeletal induced was a set of cytoskeletal genes that may potentially mediate shape change and migratory behavior of activated DC. The induction of signaling genes and transcription factors in the middle phase may be involved in preparing the DC to be receptive to regulatory signals in the lymphatics and lymph nodes. In addition, several antigen processing and presentation genes were induced to high levels in a sustained fashion. Genes involved in generating reactive oxygen species (ROS) were induced across the time course, which suggests that infecting organisms are killed throughout DC maturation and migration. Finally, during the late phase, chemokine receptors known to mediate responses to lymph node chemokines, thereby mediating DC migration, were up-regulated. The set of 165 genes described here thus constitutes part of a core DC response. This response is elicited independently of pathogen characteristics and unfolds as a temporally ordered cascade that modulates both innate and adaptive immune responses (FIG. 8C).

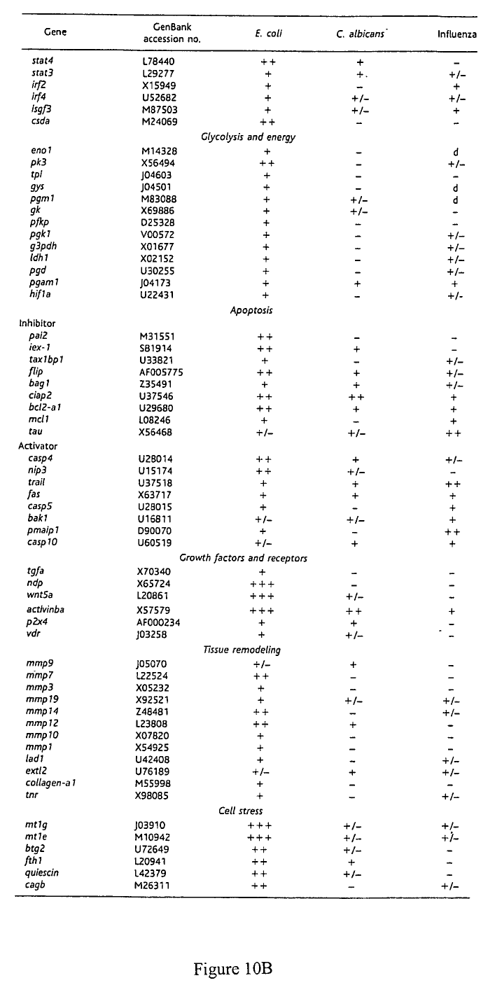

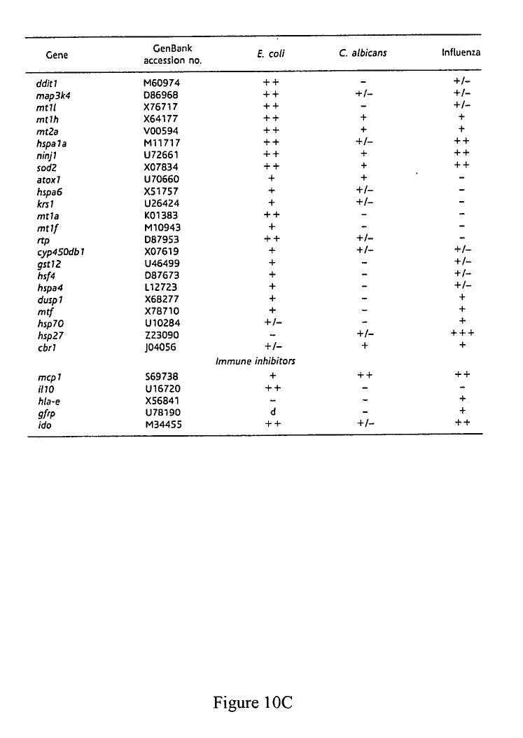

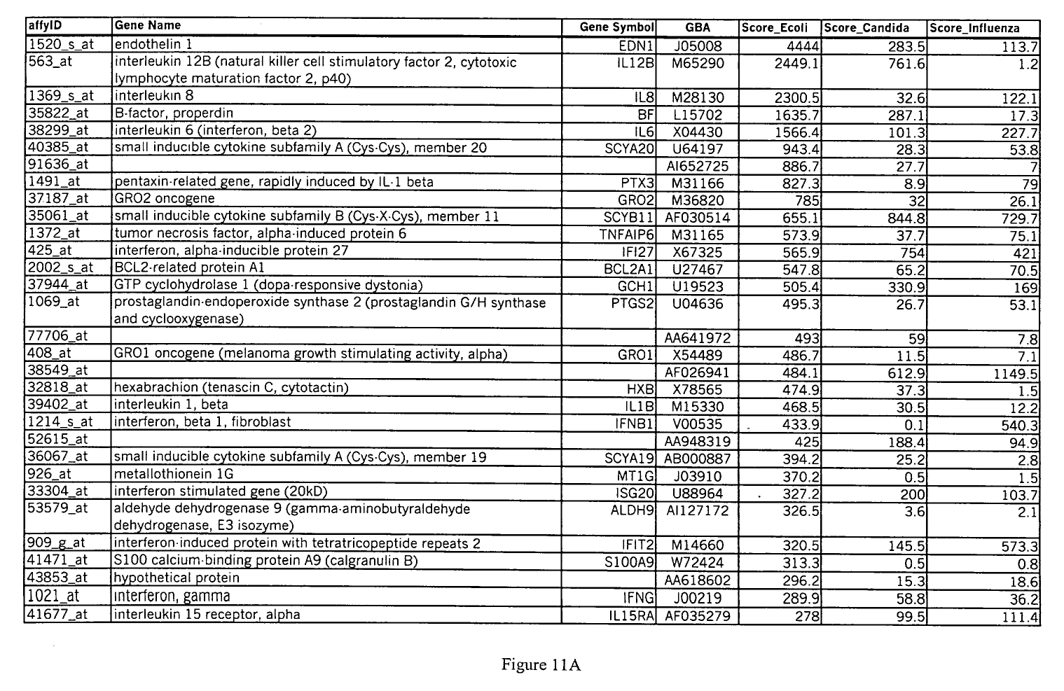

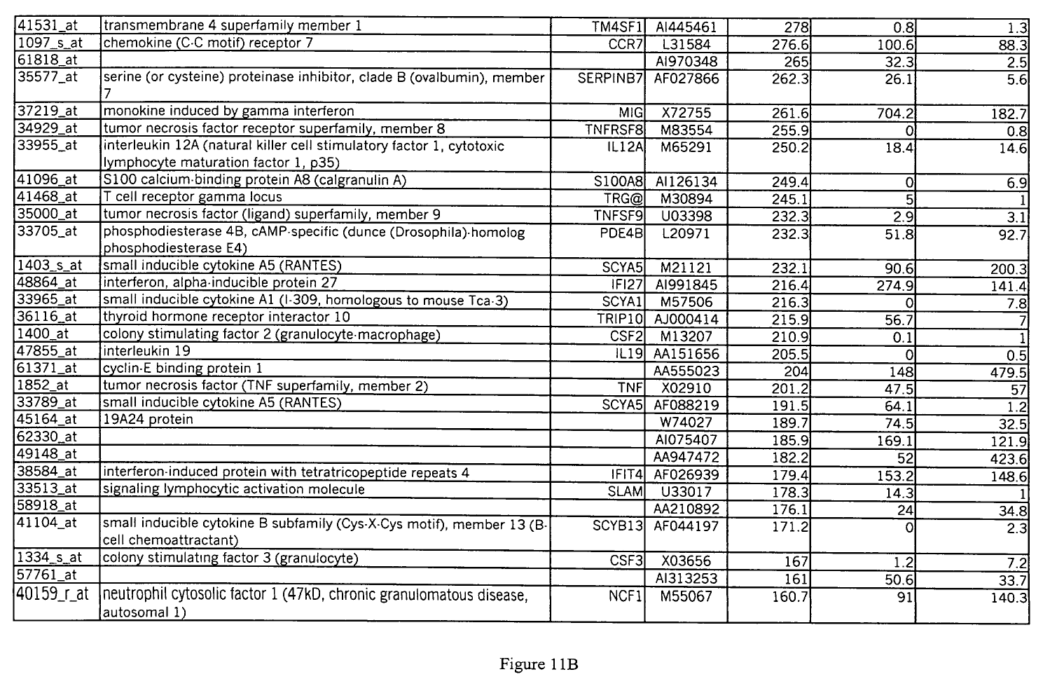

In contrast, analysis of the E. coli-specific genes (FIGS. 7E and 10A-10C) showed that DC also strongly and rapidly up-regulated most innate immune genes on the array, including inflammatory cytokines, neutrophil- and monocyte-attracting chemokines, and prostaglandin pathway components. This potent inflammatory response probably is partially counteracted by interleukin-10 (IL-10) that is induced in the middle phase. At later times, genes that regulate the adaptive immune response were induced, including T cell stimulating genes, secreted cytokines, and a subset of chemokines that are thought to attract naive T_{H} 2 T helper cells (von Andrian and Mackay, New Engl. J. Med. 343:1020-1034 (2000)). An unexpected class of cytokine receptors that share a common γ chain (IL-2R, IL-7R, IL-15R, and IL-4R) were also induced. The expression of these receptors may allow DC to respond to lymphocyte-derived interleukins within the lymph node.

All these immunostimulatory responses may be enhanced through induction of additional induced gene families (FIGS. 10A-10C), for example, cell stress genes that modulate levels of antimicrobial ROS, anti-apoptotic genes that may extend the lifetime of the infected DC (O'Reilly and Strasser, Inflamm. Res. 48:5-21(1999)), and the late-expressing matrix metalloproteases that may allow processing of cytokines and DC migration to lymph nodes (Murphy and Gavrilovic, Curr. Opin. Cell Biol. 11:614-621 (1999)). Genes with undefined roles in DC function were also regulated by E. coli, including signaling molecules, transcription factors, adhesion molecules, and many of the glycolytic genes. HIF1α, a known transcription factor of glycolysis genes, was also up-regulated (Wenger, J. Exp. Biol. 203:1253-1263 (2000)). Collectively, these diverse changes of gene expression in response to E. coli and LPS reflect a significant cellular and immunological reprogramming of the DC.

Relative to the response of DC to E. coli, their response to C. albicans was greatly attenuated in many functional categories and constituted a subset of the E. coli response, with a much smaller number of immune genes and with no robust C. albicans-specific genes (FIGS. 7A, 7B and 10A-10C). Because many of the immune genes are known to be regulated by the transcription factor NF-κB (Ghosh, et al., Annu. Rev. Immunol. 16:225-260 (1998)), this difference may be partially explained by the relatively weak NF-κB up-regulation (FIGS. 10A-10C).

DC regulated a large number of genes in response to influenza, comparable to the number regulated in response to E. coli. However, the innate immune response was relatively weak and completely devoid of genes capable of stimulating neutrophils, as confirmed by a neutrophil chemotaxis assay. The adaptive response to influenza was also distinct from the response to E. coli. The antiviral genes those encoding interferon (IFN) α and β were strongly induced, as were the interferon-inducible chemokine genes (FIGS. 10A-10C). This suggests possible effects on induction and migration of naive T_{H} 1 cells ((von Andrian and Mackay, New Engl. J. Med. 343:1020-1034 (2000)). An important subset of genes induced by influenza are linked with the inhibition of the immune response at certain stages. These include proapoptotic genes that may lead to early death of infected cells (O'Reilly and Strasser, Inflamm. Res. 48:5-21 (1999)) as well as genes encoding mcp-1, which can block IL-12 production in macrophages (Braun, et al., J. Immunol. 164:3009-30017 (2000)); HLA-E, which can inhibit natural killer cells (Tomasec, et al., Science 287:1031 (2000)); Gfrp, a close homolog of a protein that inhibits NO synthesis (Milstien, et al., J. Biol. Chem. 271:19743-19751 1996)); and IDO, which can inhibit T cell activation (Hwu, et al., J. Immunol. 164:3596-3599 (2000)). Influenza also modulated the expression of a large set of genes involved in diverse cellular functions and whose contribution to pathogen-host interactions may not have been studied previously.

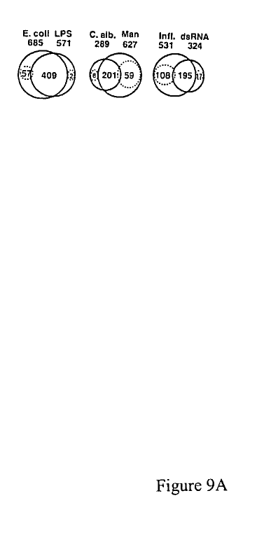

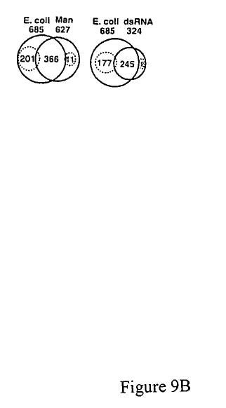

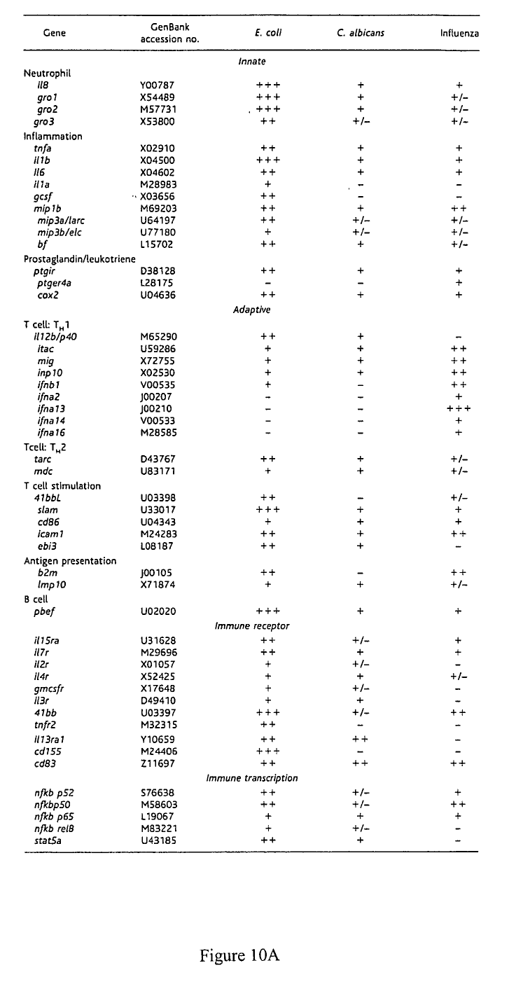

To further dissect the ability of DC to discriminate pathogens, the sufficiency of individual pathogen components to elicit these differential pathogen responses was investigated. Despite additional active molecules known to be present on bacteria, LPS was able to mimic and account for almost the entire bacterial response (FIG. 9A). Unexpectedly, the fungal component mannan mimicked the magnitude and biological character of the bacterial response more closely than it did the fungal or viral response profiles (FIGS. 9A and 9B). Although dsRNA was a less potent stimulator of the bacterial response, it did elicit a strong innate response comparable to that induced by bacteria, and at the same time elicited aspects seen in the viral response (FIGS. 9A and 9B). Thus, all three components were able to elicit the expression of many innate immune genes as well as most of the genes in the common pathogenresponse. This finding shows that the core DC program can be triggered by multiple stimuli with diverse molecular structures.

Genome-scale studies of DC transcriptional responses have allowed us to demonstrate the existence of common and differential pathogen recognition pathways. Although there have been reports of changes in gene expression in DC in response to LPS, the response to pathogens has not been thoroughly investigated (Hashimoto, et al., Blood 96:2206-2214 (2000); Dietz, et al., Biochem. Biophys. Res. Commun. 275:731-738 (2000)). Differential immune responses to pathogens have been described in clinical and animal studies (Ashman and Papadimitriou, Microbiol. Rev. 59:646-672 (1995); Ludwig, et al., Viral Immunol. 12:175-196 (1999); Reis e Sousa, et al., Curr. Opin. Immunol. 11:392-399 (1999)), and results described herein show that these responses are reflectedby changes in DC gene expression.

The temporal cascade of gene expression in the common response to pathogens, including Gram-positive Staphylococcus aureus (data not disclosed), accounts for core DC functions and delineates the essential role of DC in linking innate recognition of pathogens with antigen presentation and the development of an adaptive T cell response (Cella, et al., J. Exp. Med. 189:821-829 (1999); Bhardwaj, et al., J. Clin. Invest. 94: 797-807 (1994); Rescigno, et al., Proc. Natl. Acad. Sci. U.S.A. 95:5229-5234 (1998); Hemmi, et al., Nature 408:740-745 (2000). The existence of this common response reflects a convergence of pathways from receptors that are known to distinguish some of these components and pathogens. In contrast, the presence of pathogen-specific gene expression in most functional categories (including transcription factors and cytokines) suggests that distinct pathways are activated by different pathogens. These differential responses demonstrate that human monocyte-derived DC are flexible in their responses and may even exhibit a diversity of responses similar to that of the different DC subtypes (Rissoan, et al., Science 283:1183-1186 (1999); Banchereau, et al., Annu. Rev. Immunol. 18:767-811 (2000)).

The extensive plasticity of the DC observed in our experiments indicates that the concept of DC maturation cannot be simply defined by the modulation of a standard set of markers (Banchereau and Steinman, Nature 392:245-252 (1998)). Instead, work described herein indicates that DC not only are capable of generating a core response to any pathogen, but also exhibit stimulus-specific maturation and activation. For each stimulus, particular subsets of genes are modulated and lead to important physiological consequences. There is likely to be even more differential regulation in vivo, depending on DC subtype, cell interactions, and tissue location. Determining whether these unique responses are advantageous to the pathogen, or to the host, is essential for understanding host-pathogen interactions (d'Ostiani, et al., J. Exp. Med. 191:1661-1674 (2000)). Further studies of these pathogen-regulated genes may thus enhance the understanding of DC maturation and provide future targets for immunotherapy.

Summary of HU6800 Results

Stimulus-Responsive Genes

The results are as follows (p<0.00005):

E. coli: 685 genes (FIGS. 1A-1GG)

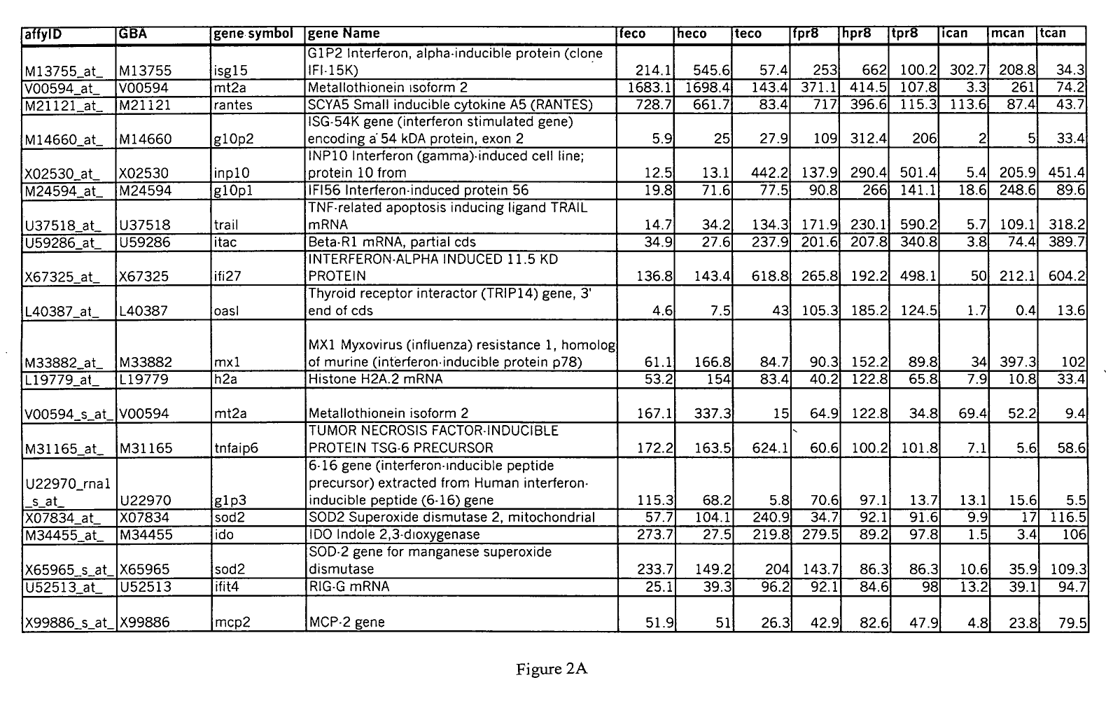

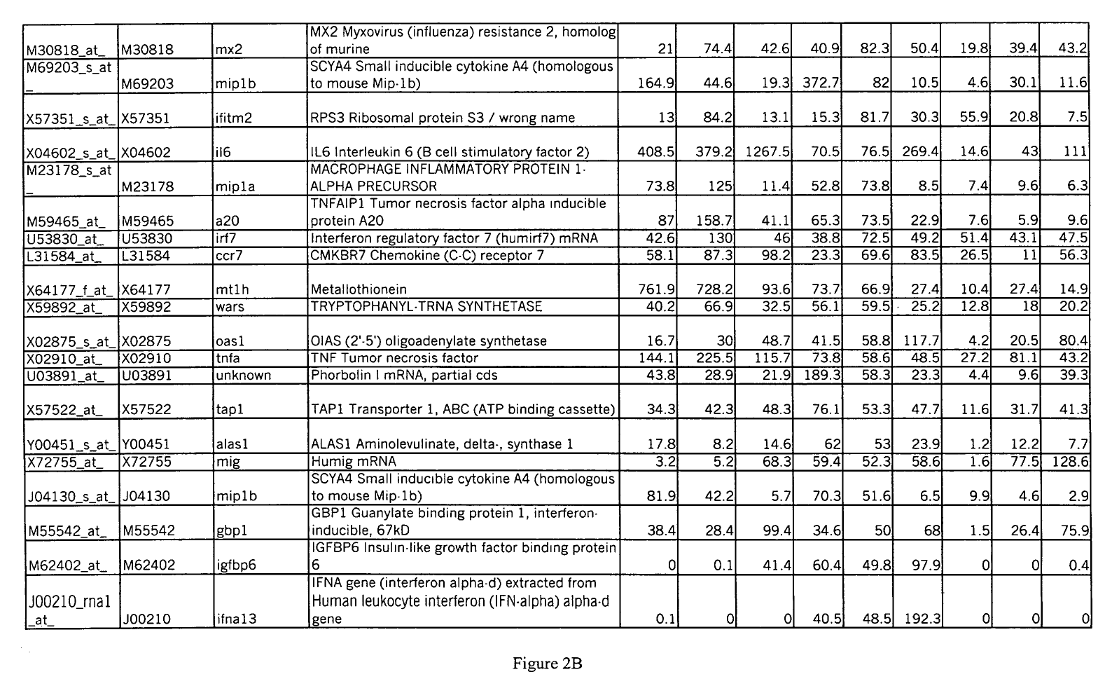

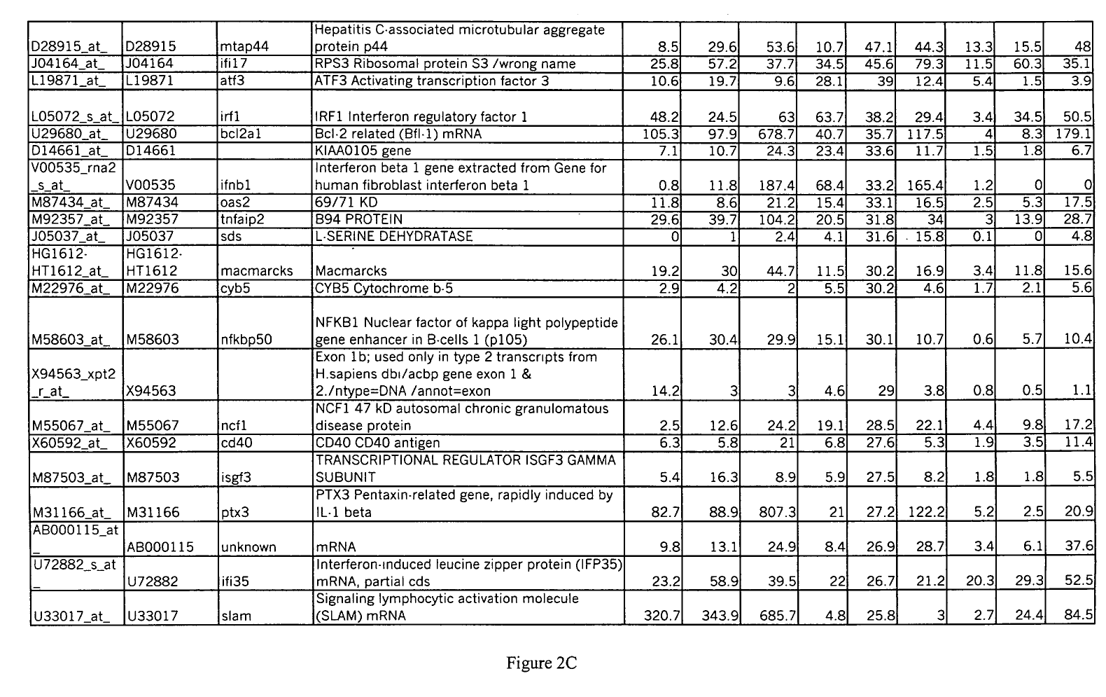

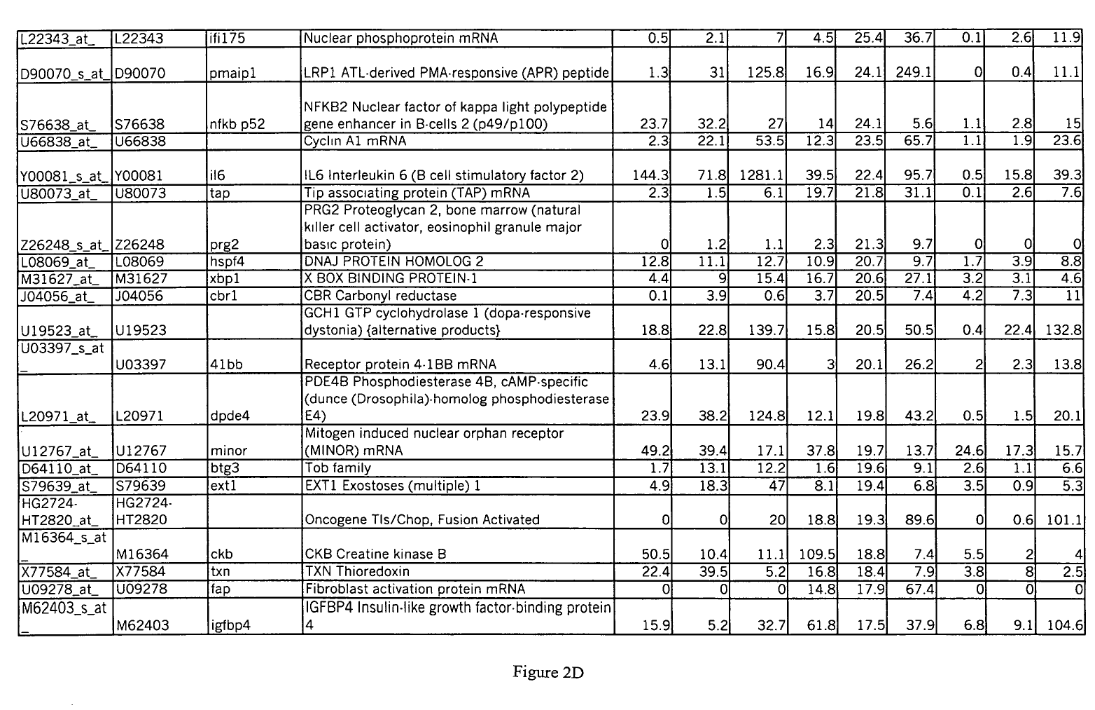

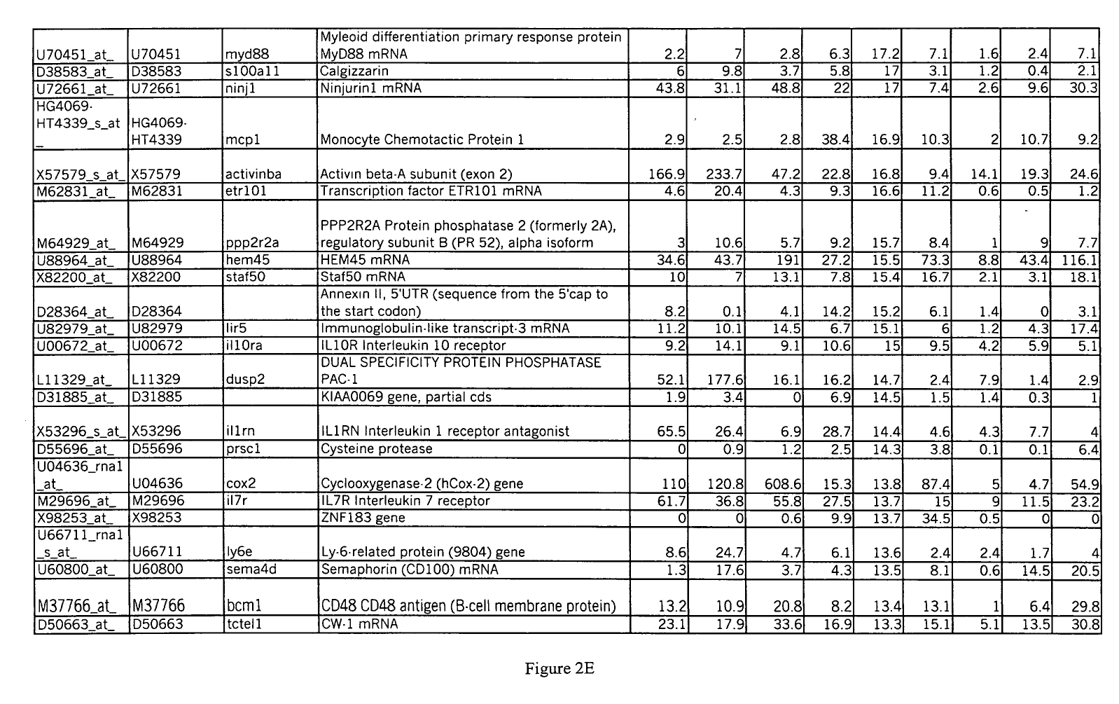

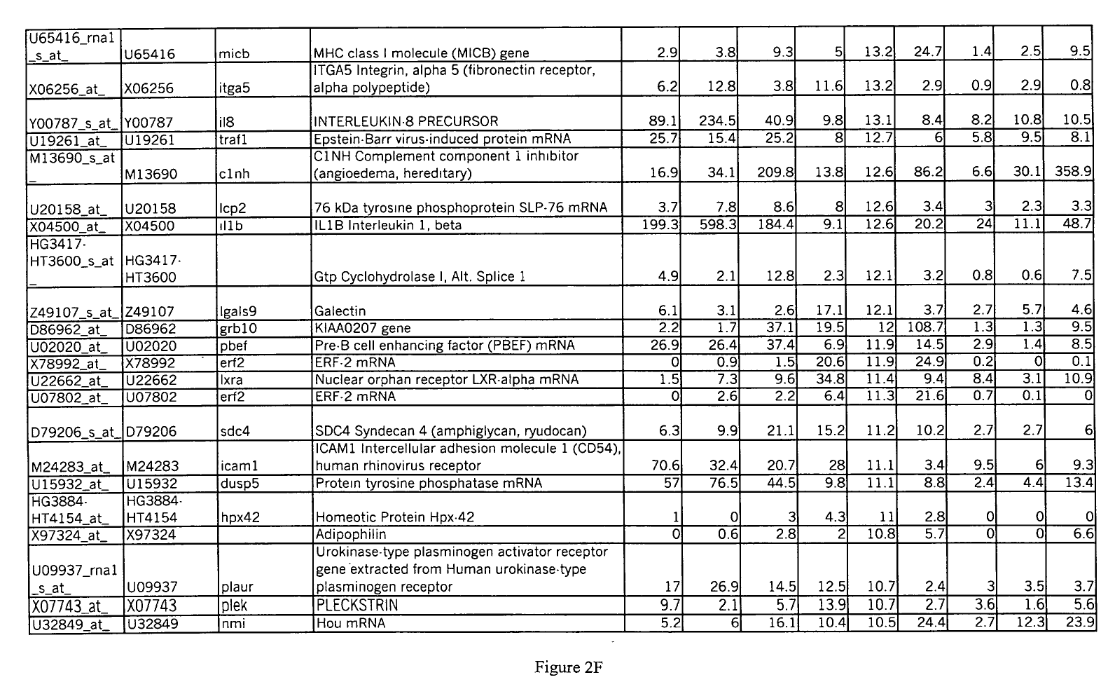

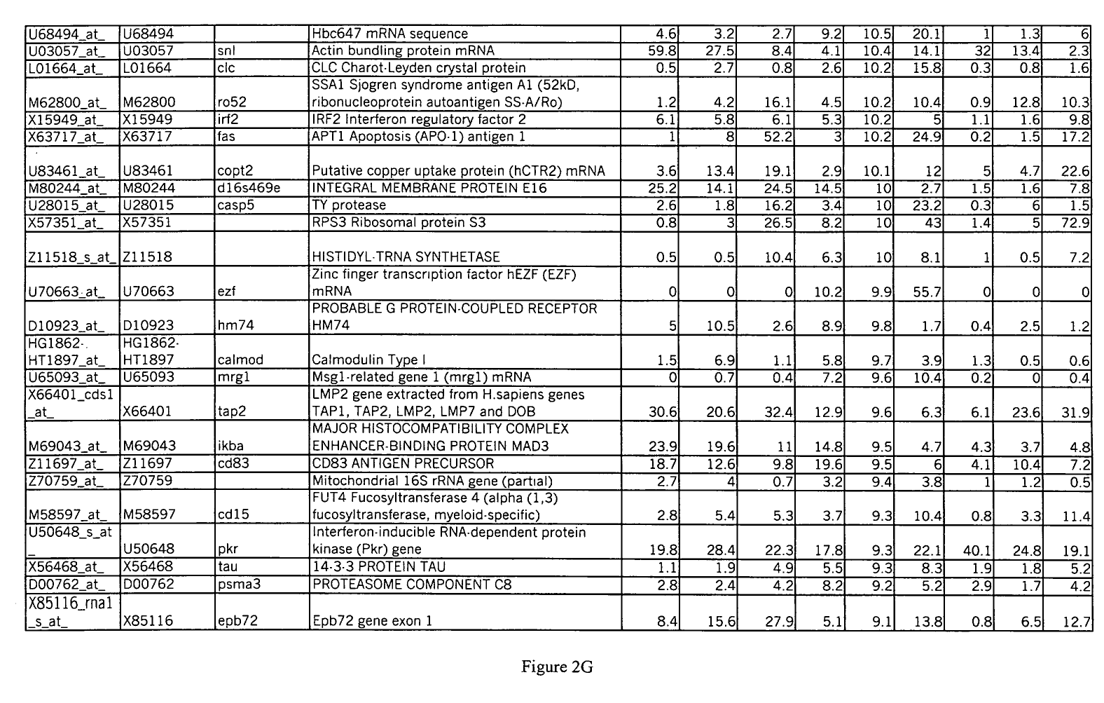

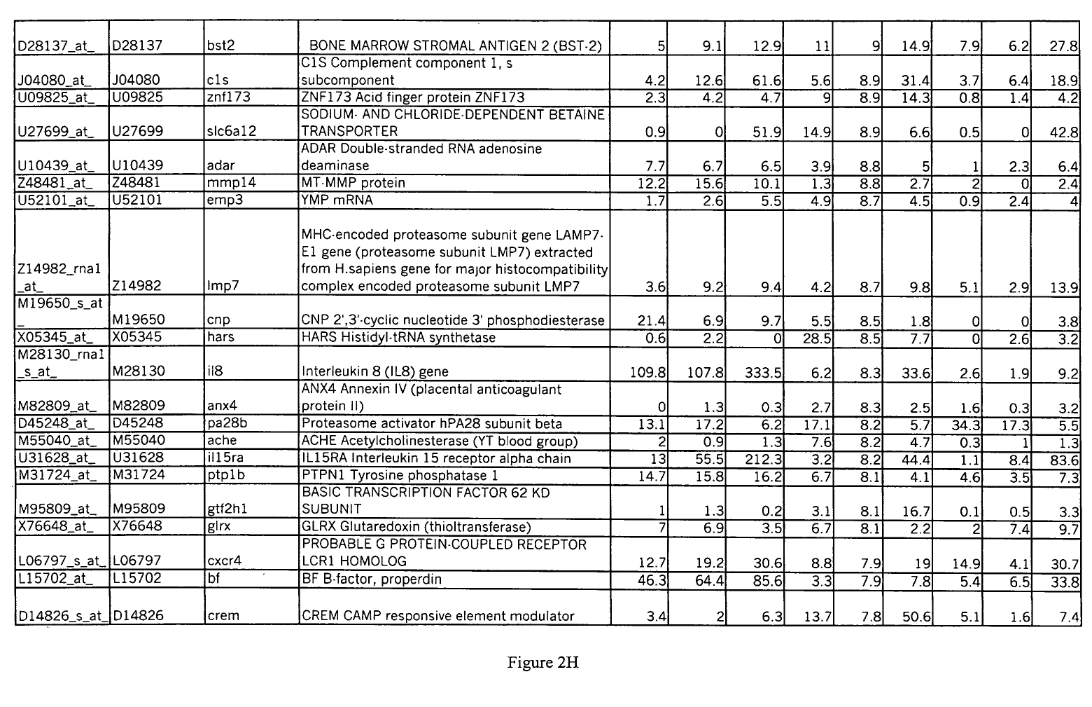

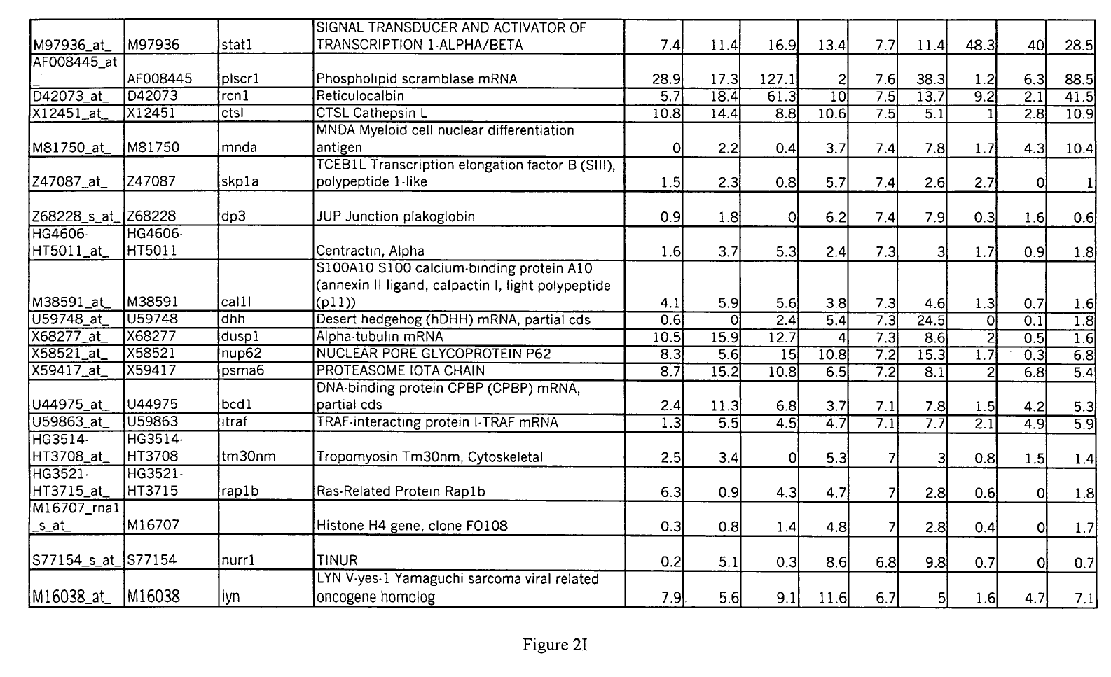

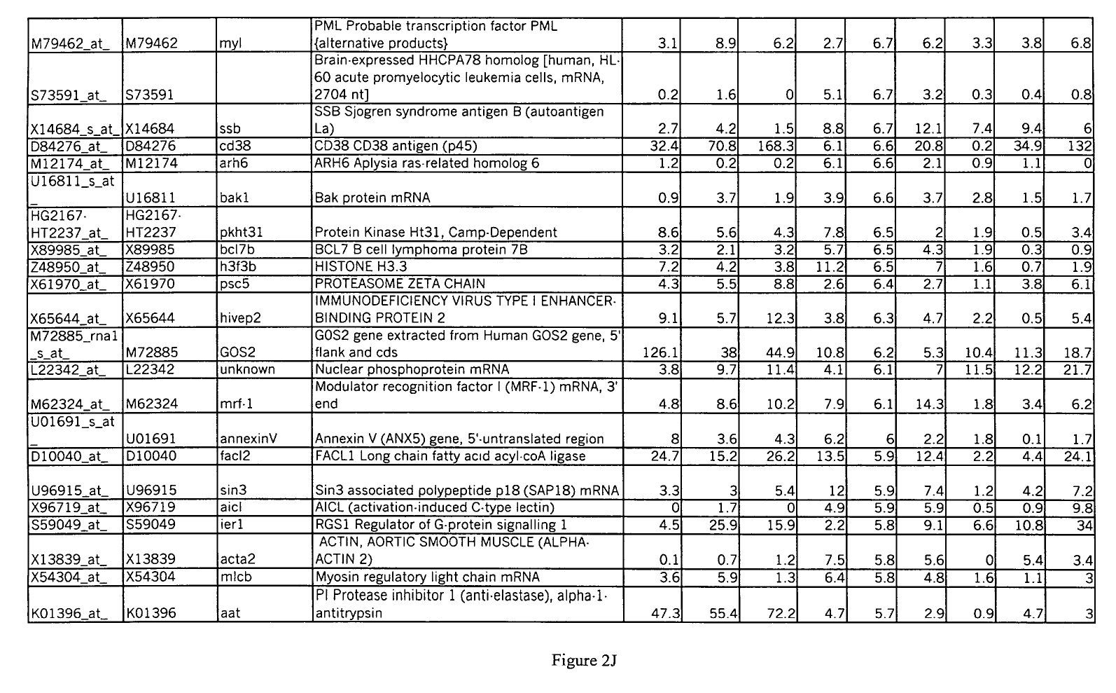

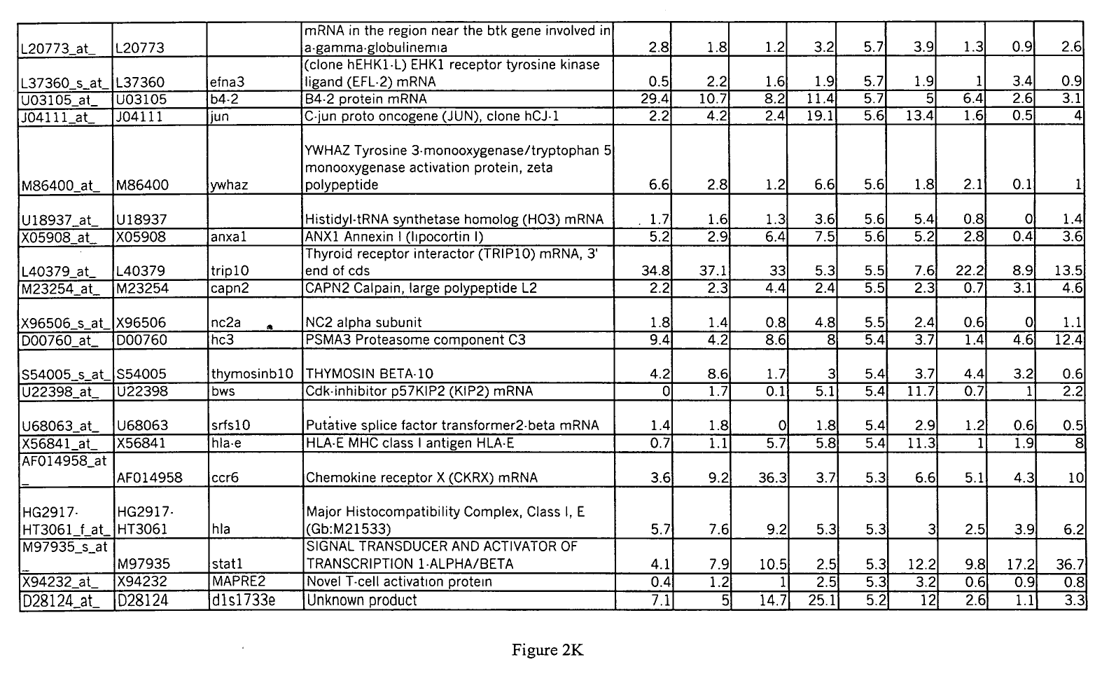

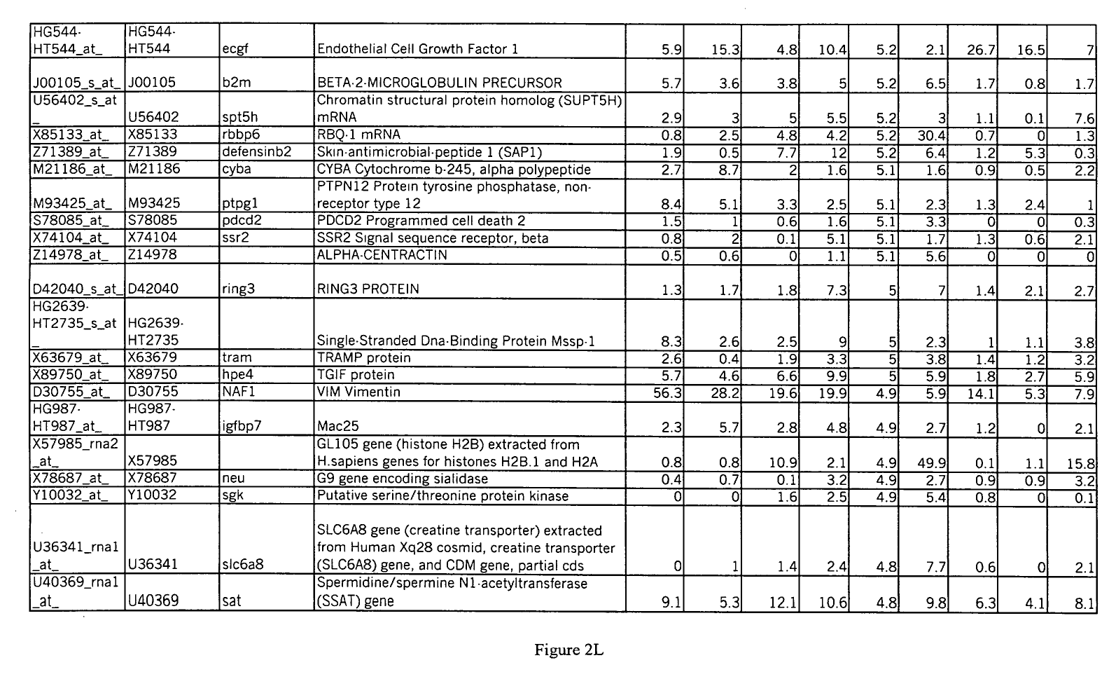

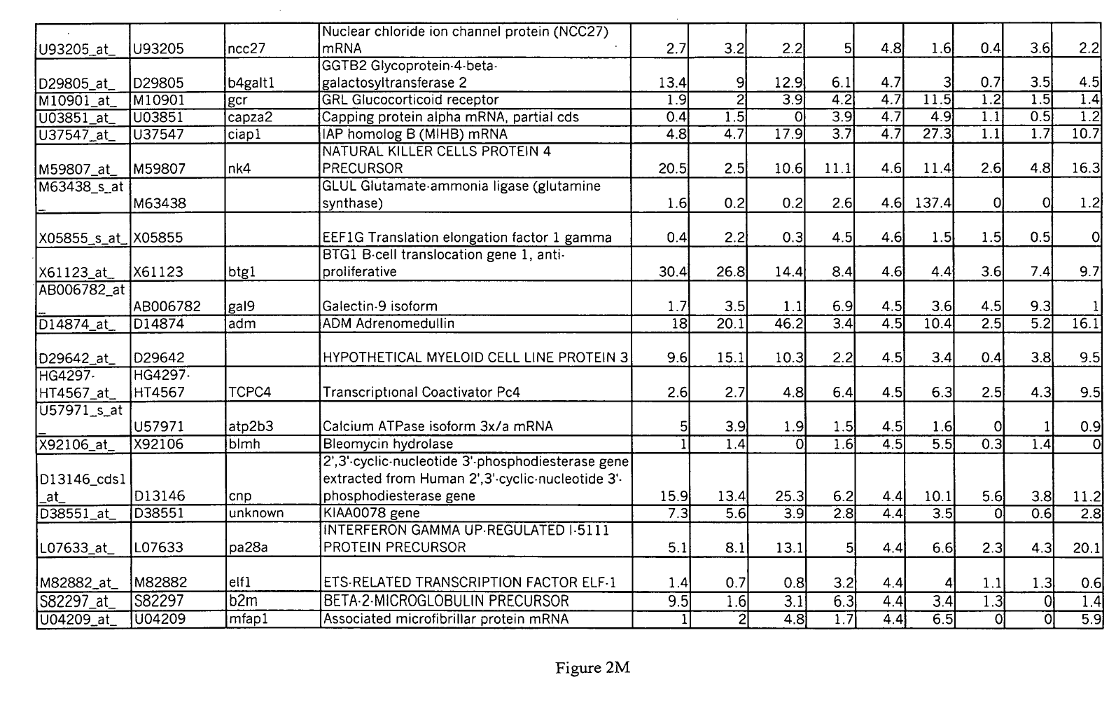

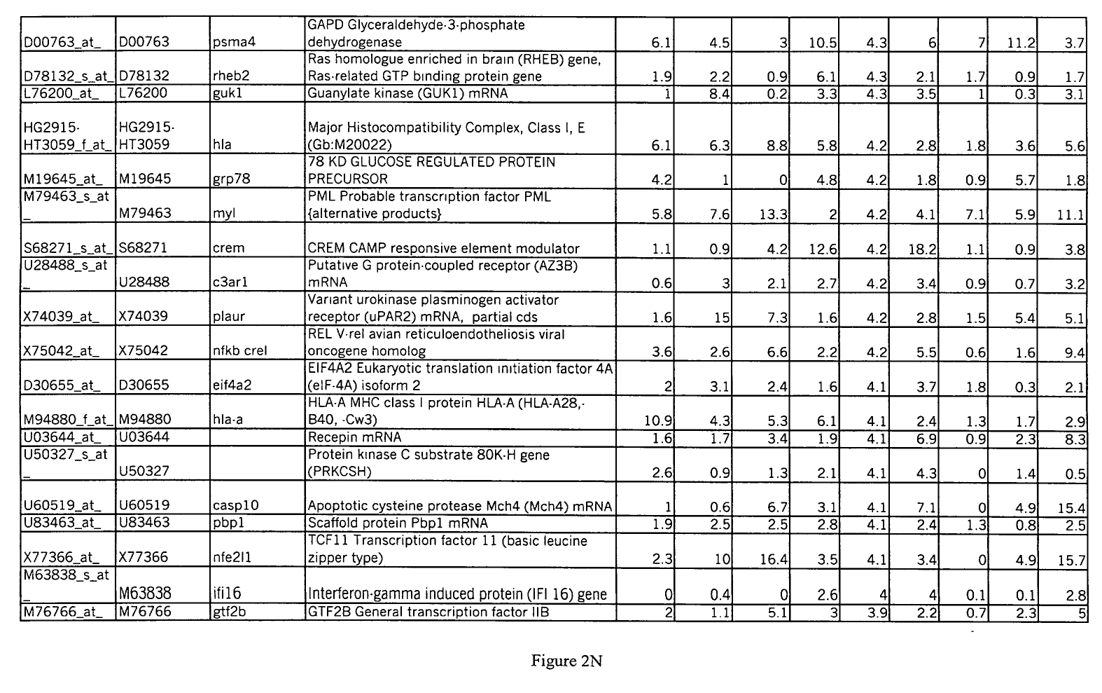

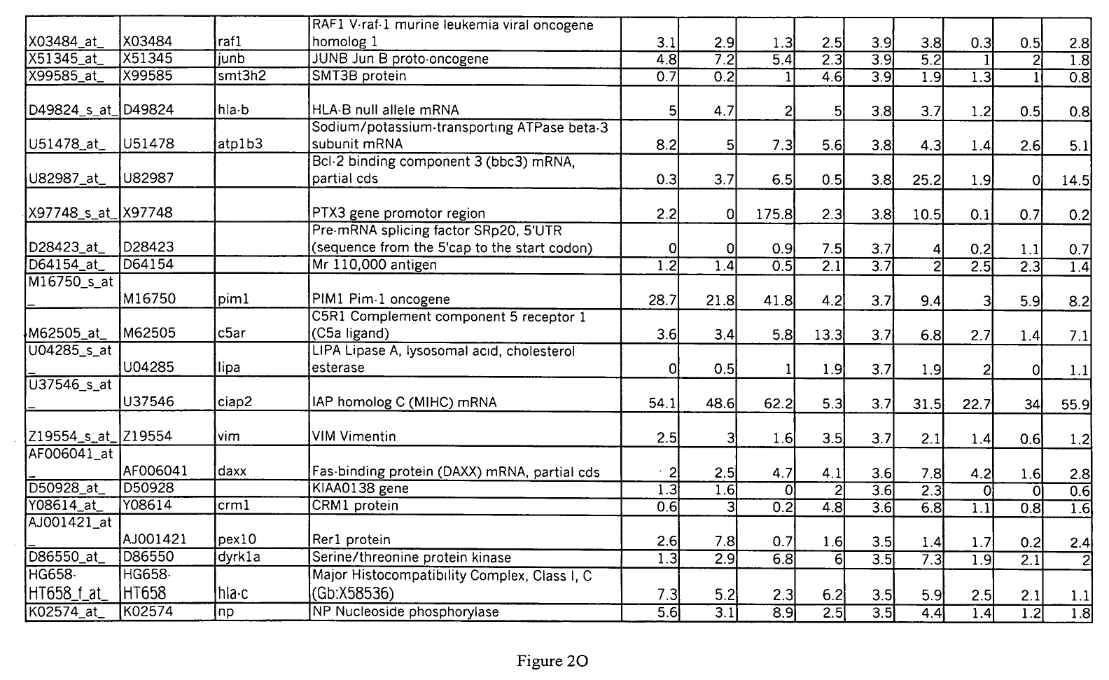

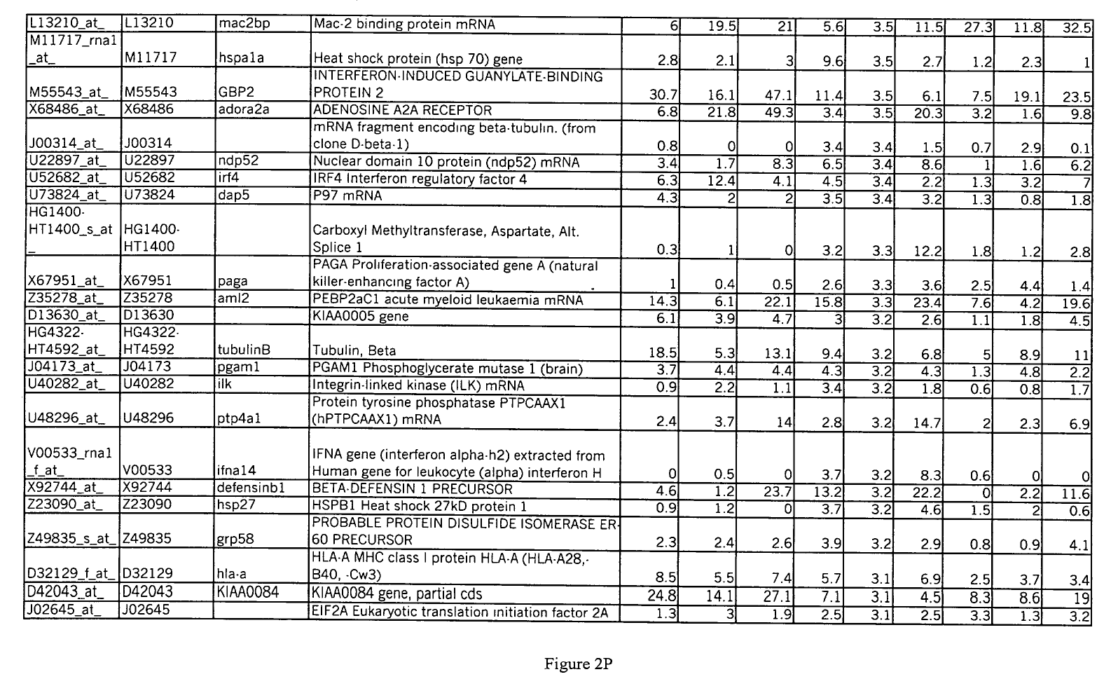

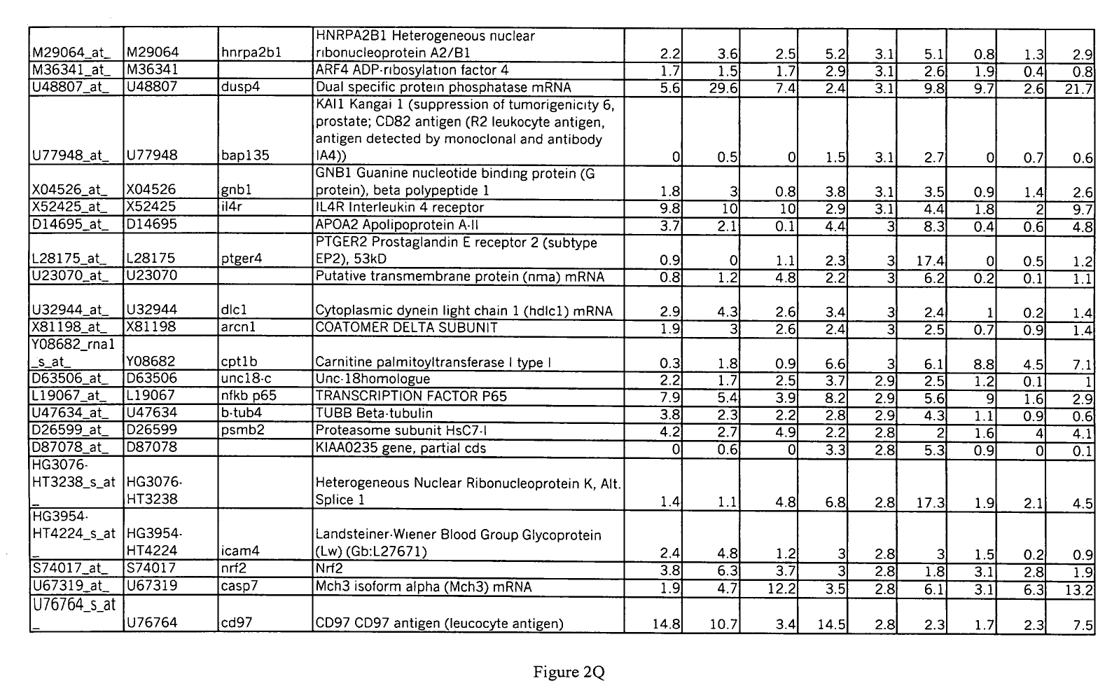

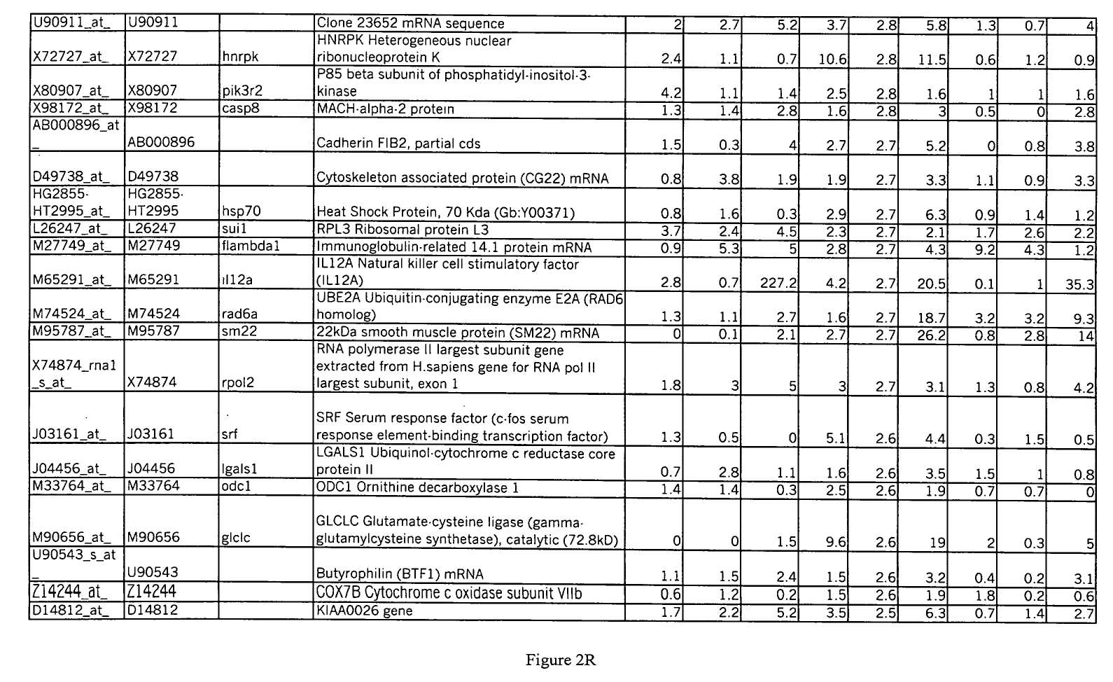

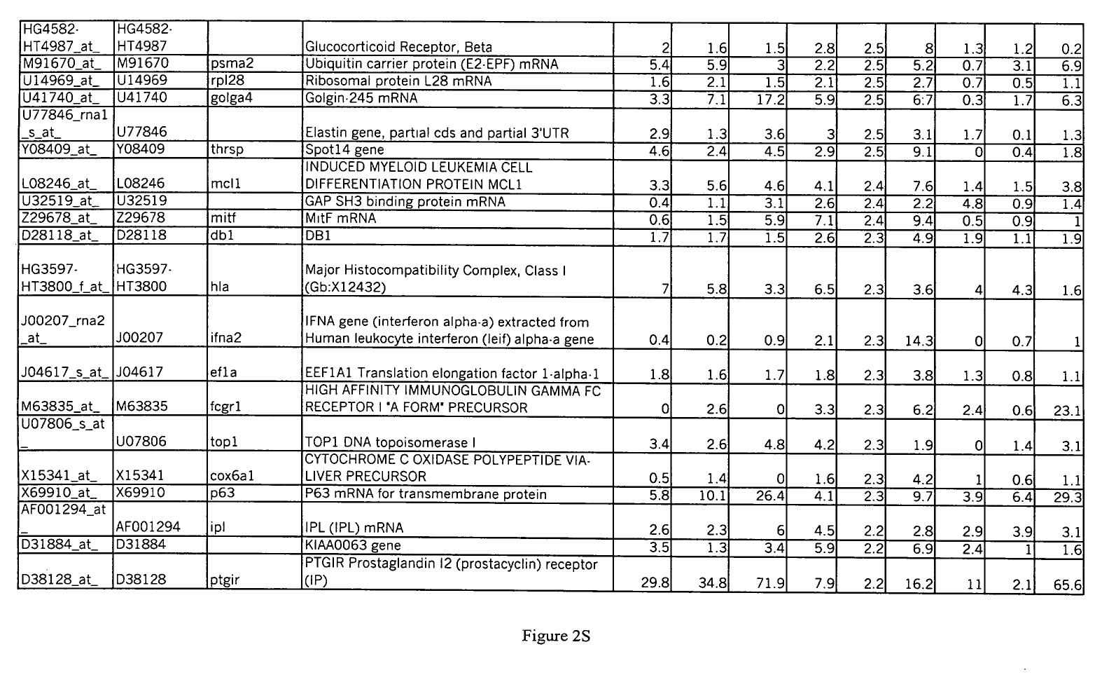

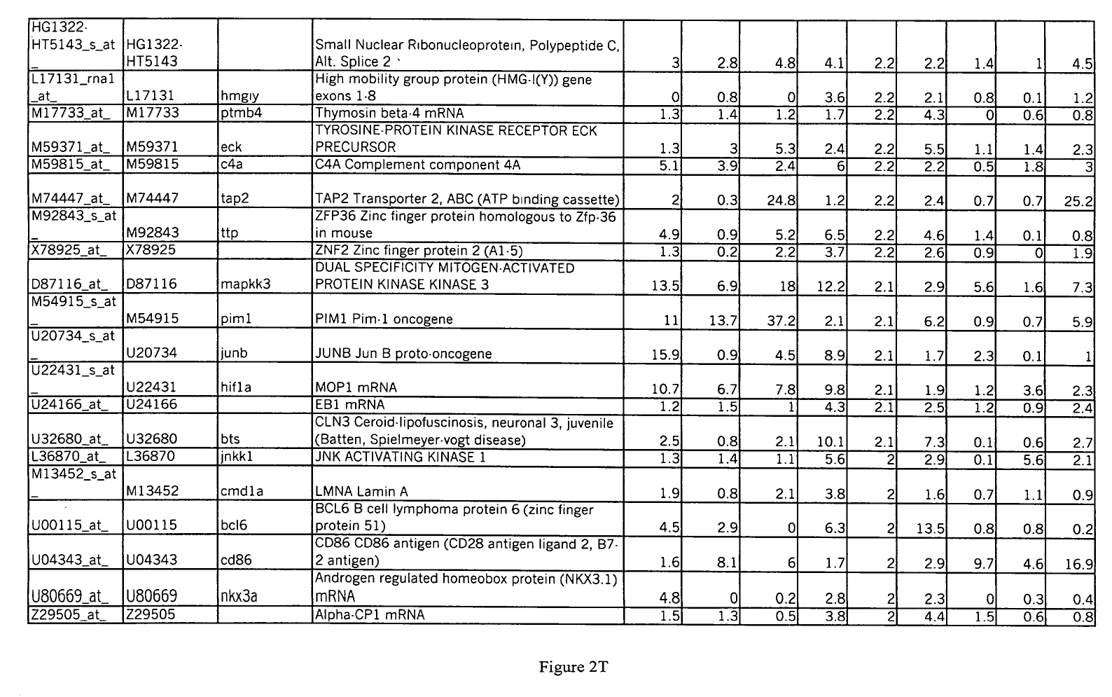

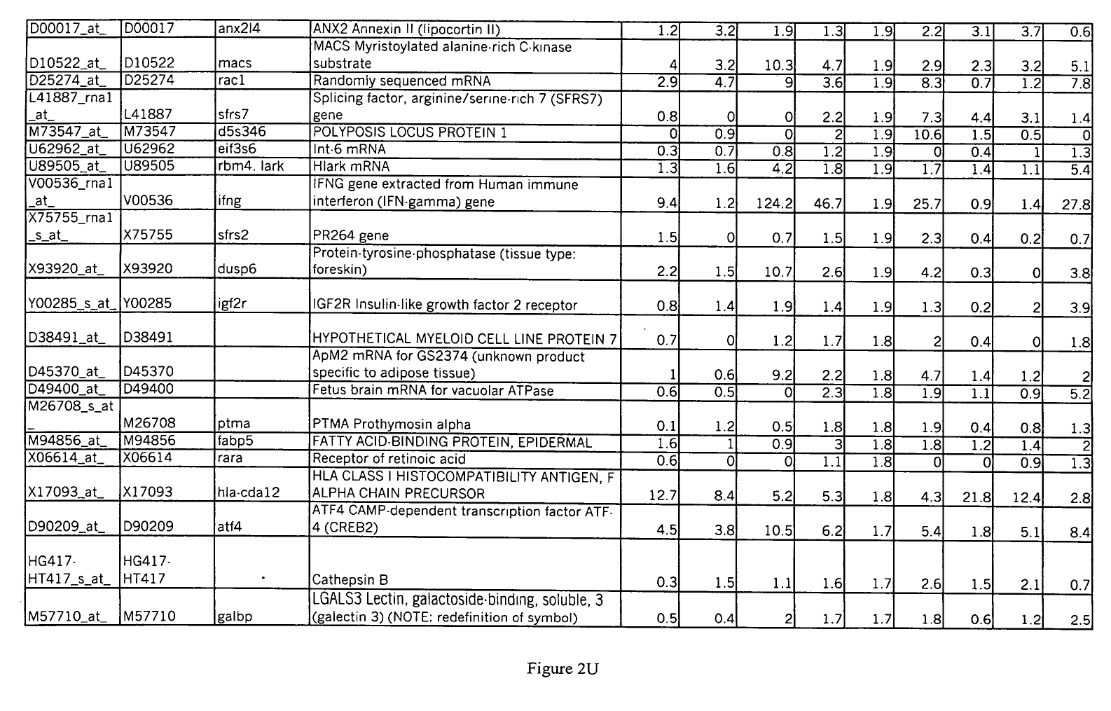

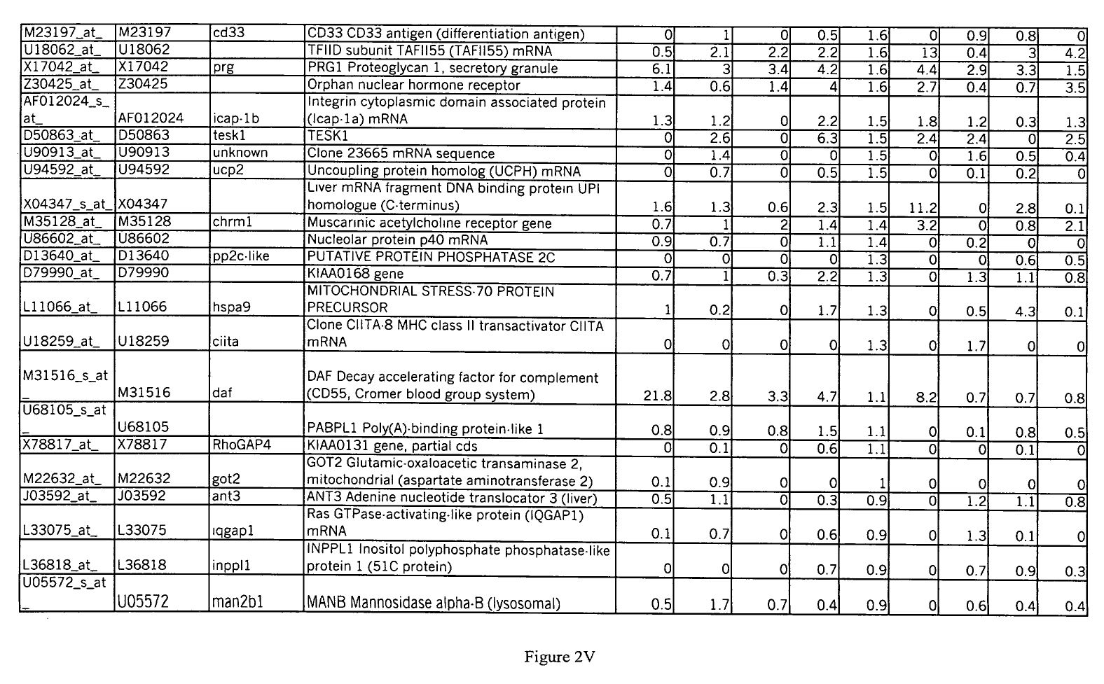

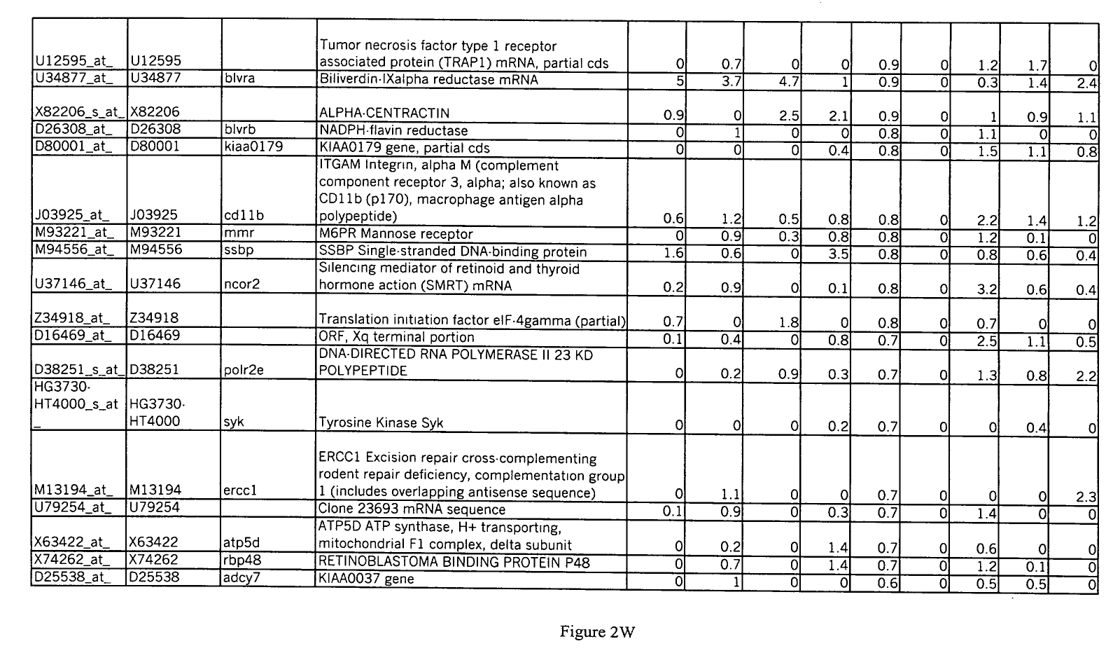

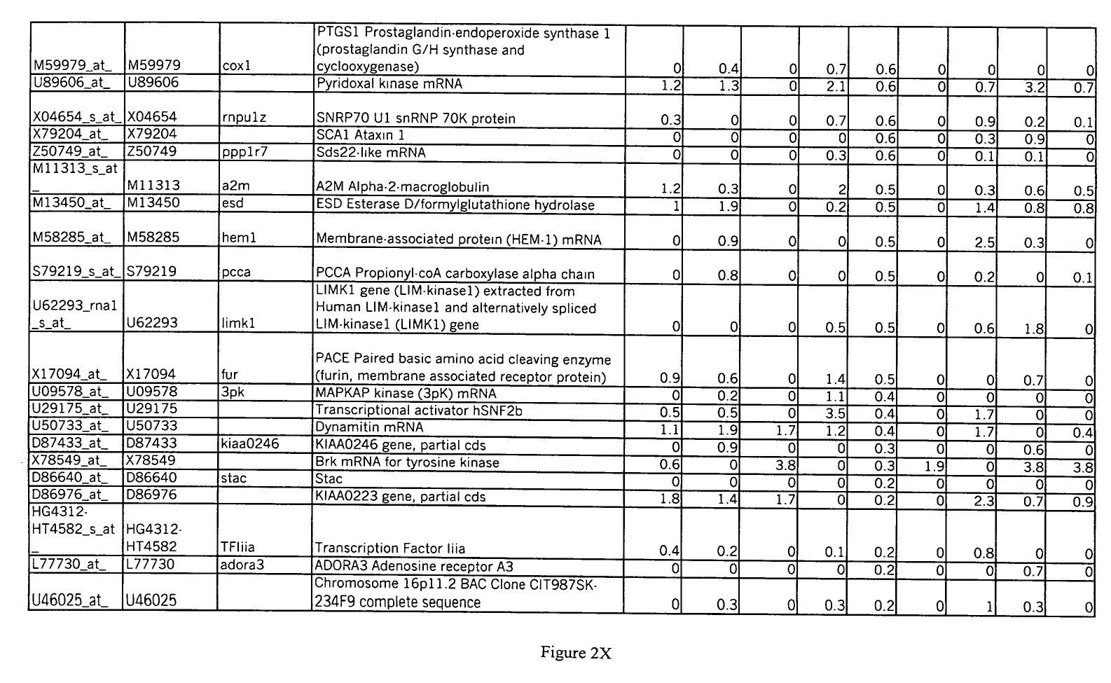

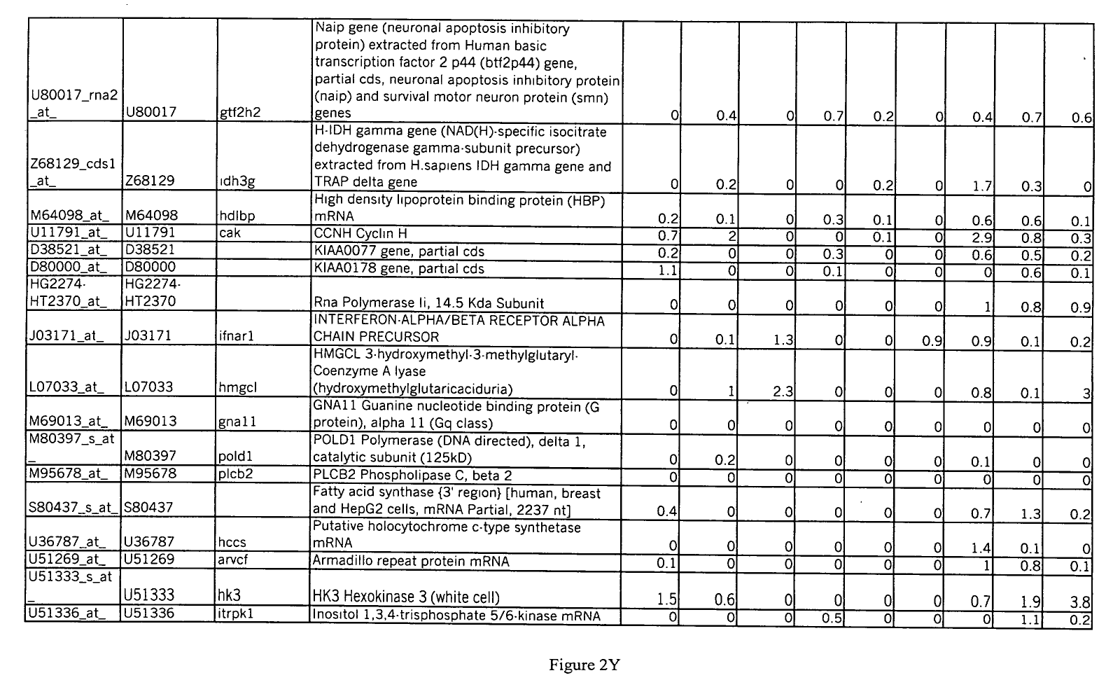

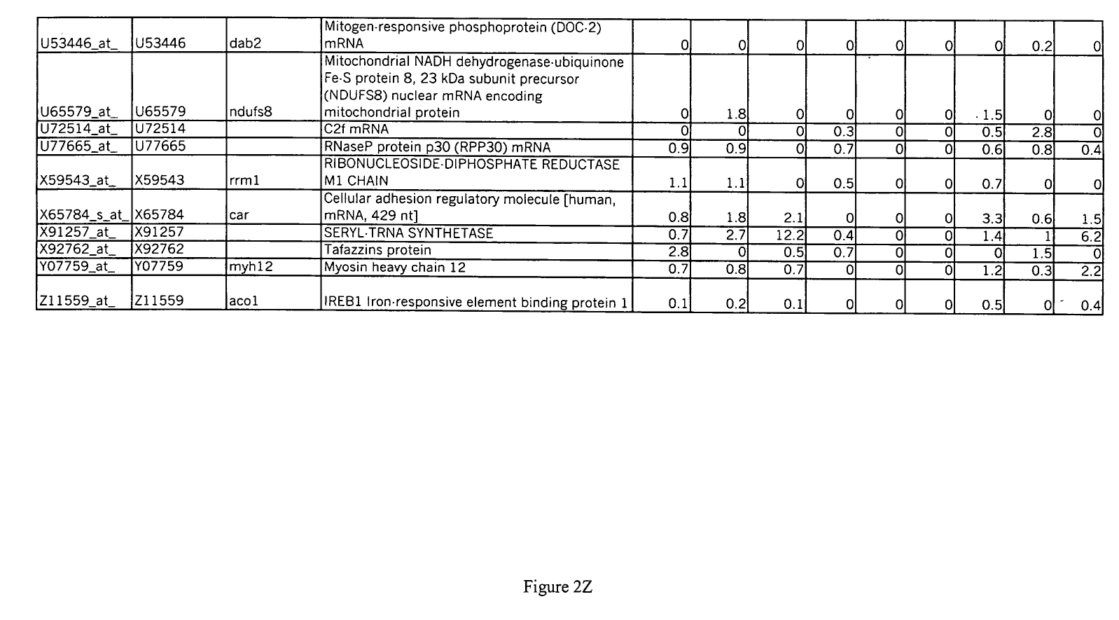

influenza: 531 genes (FIGS. 2A-2Z)

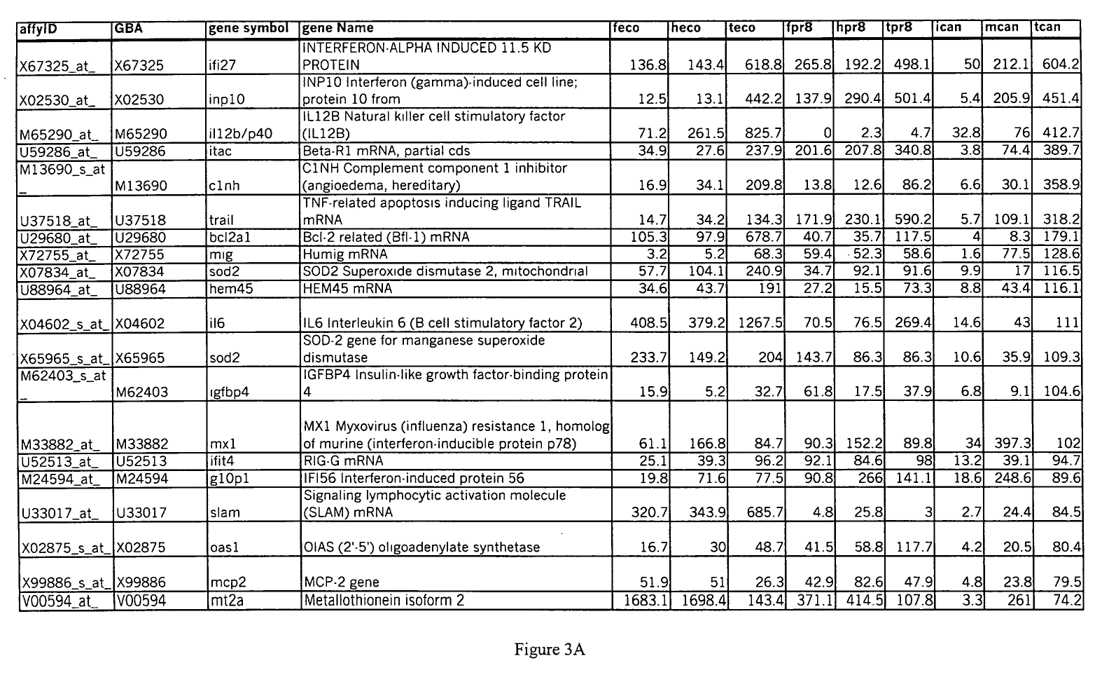

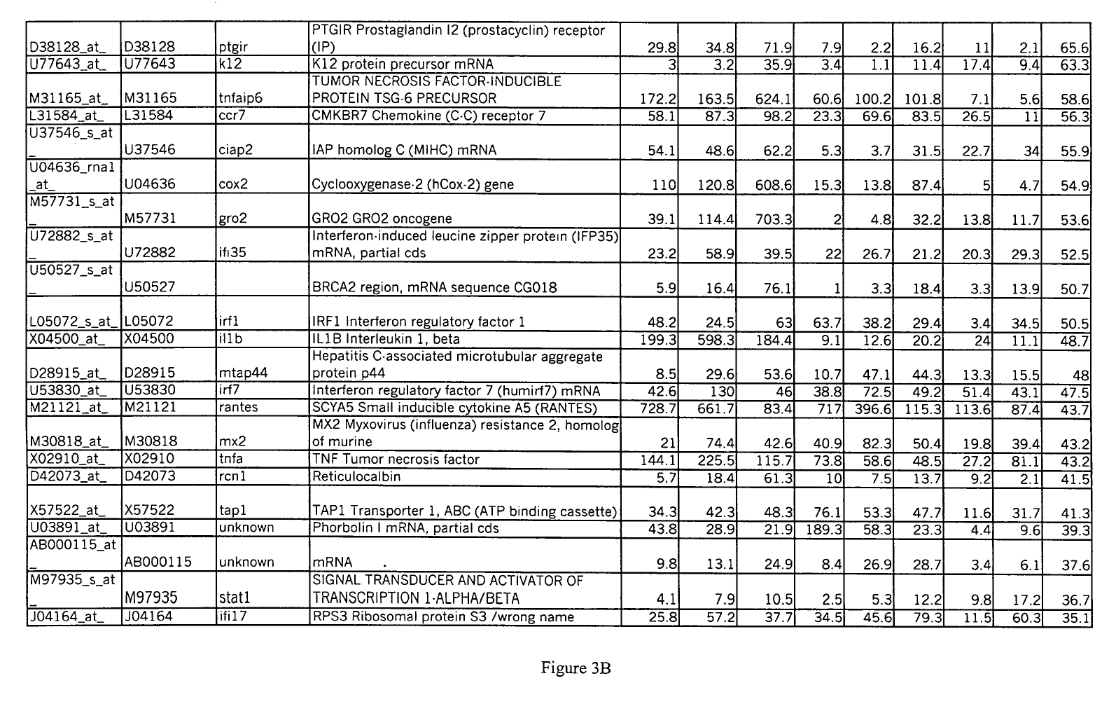

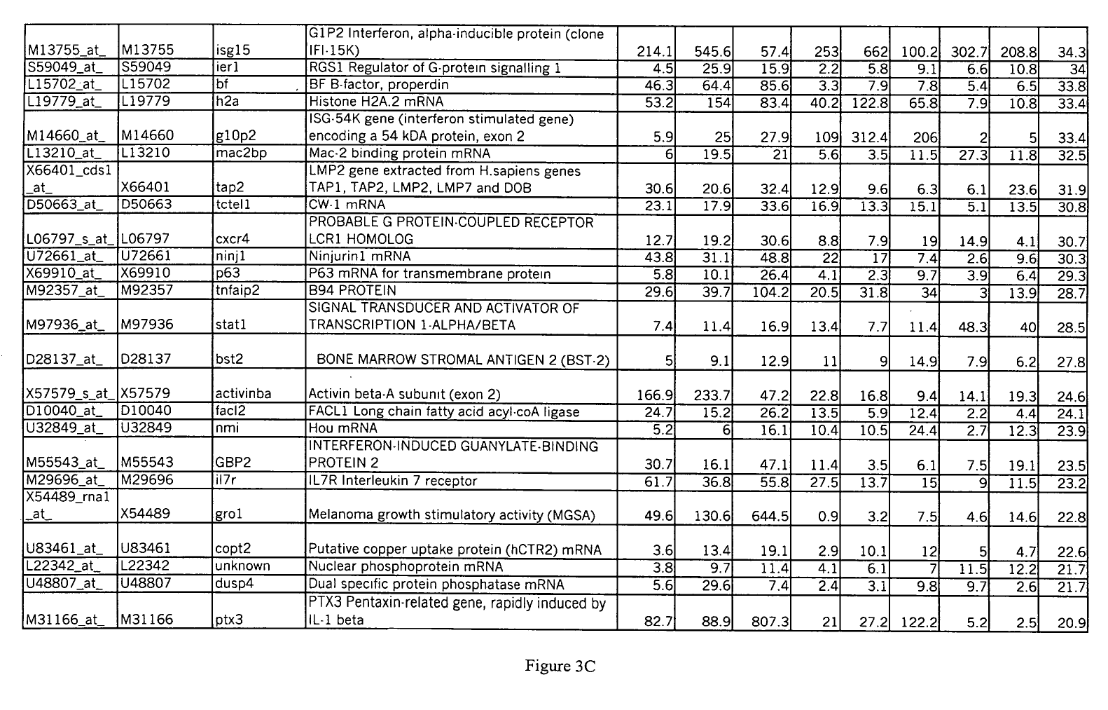

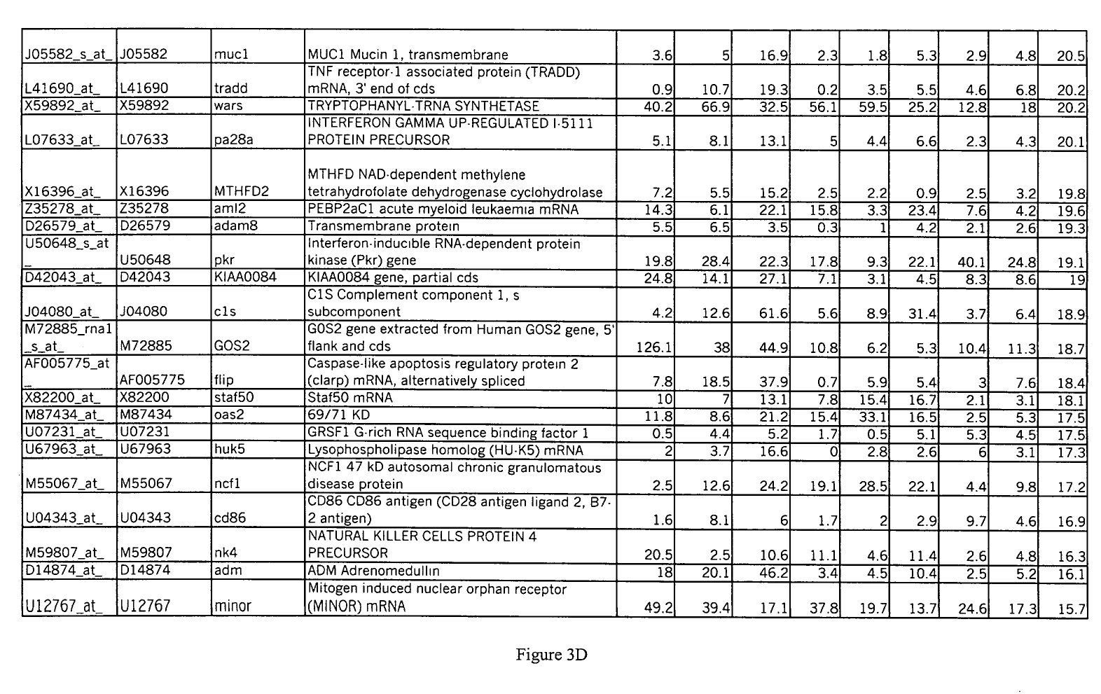

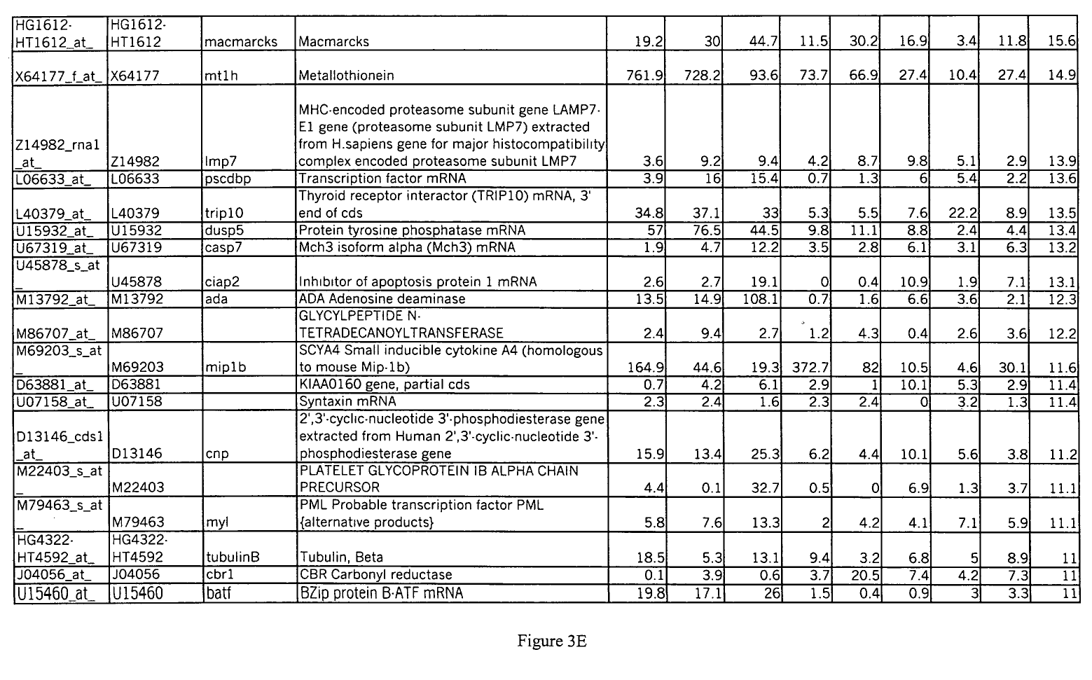

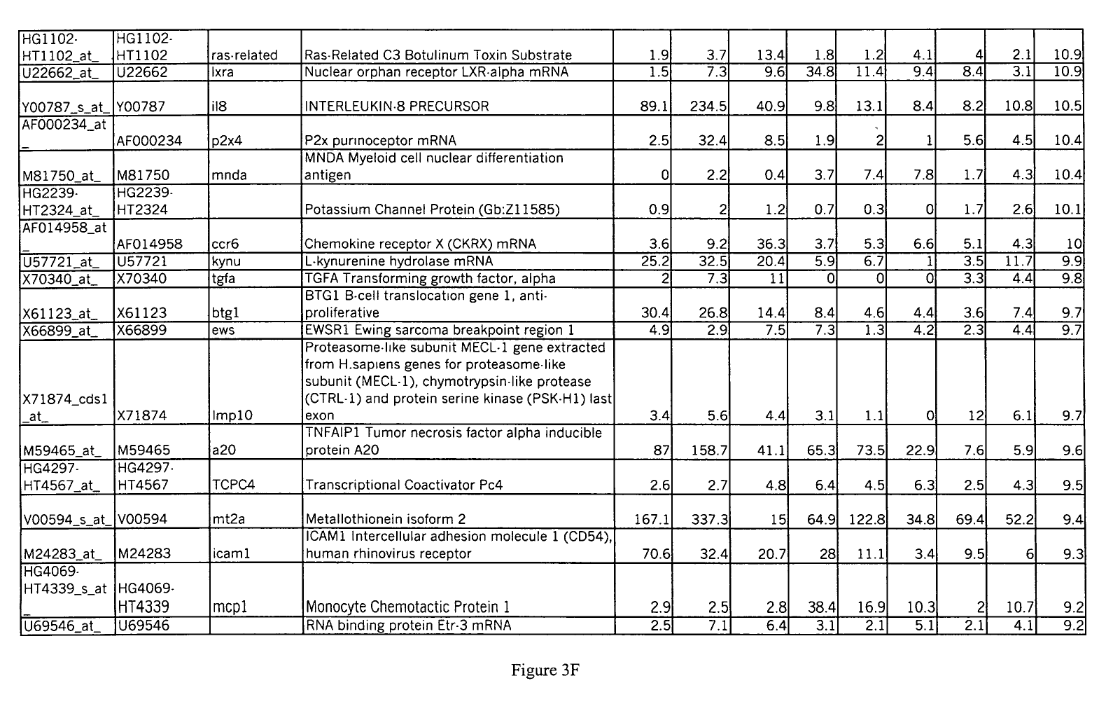

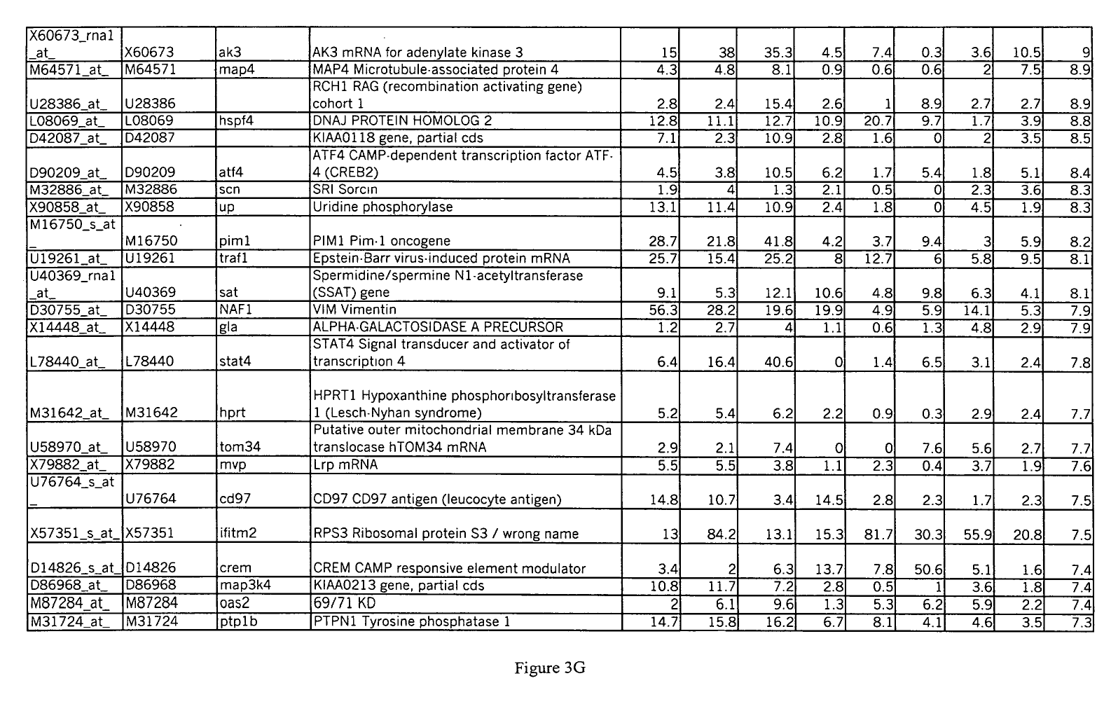

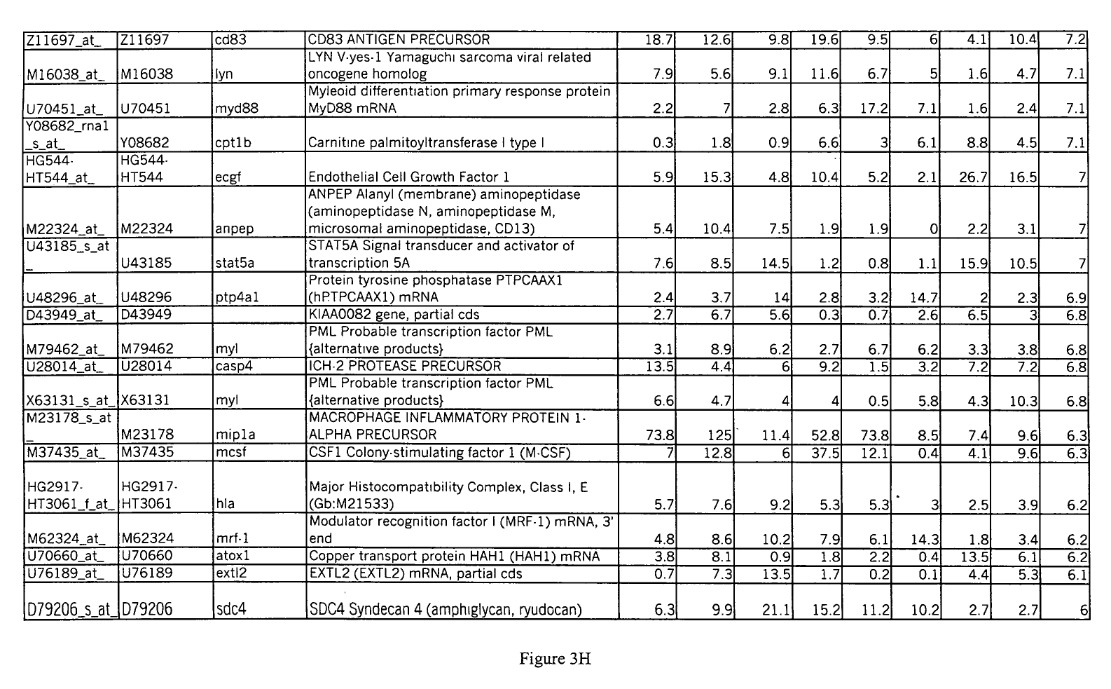

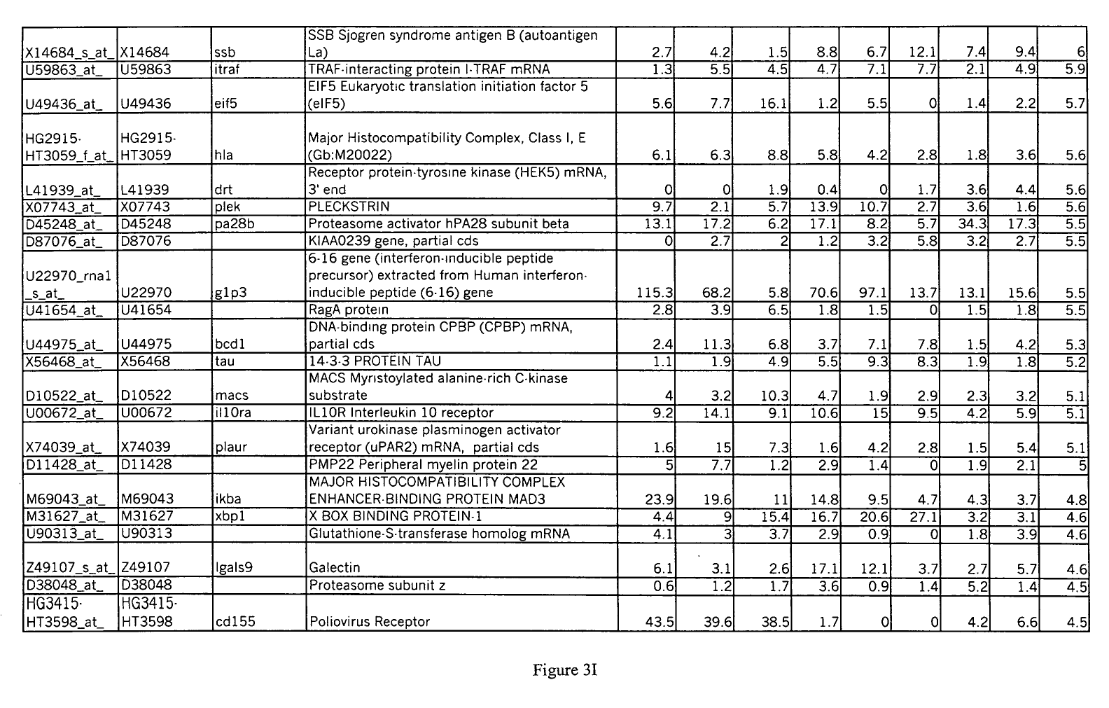

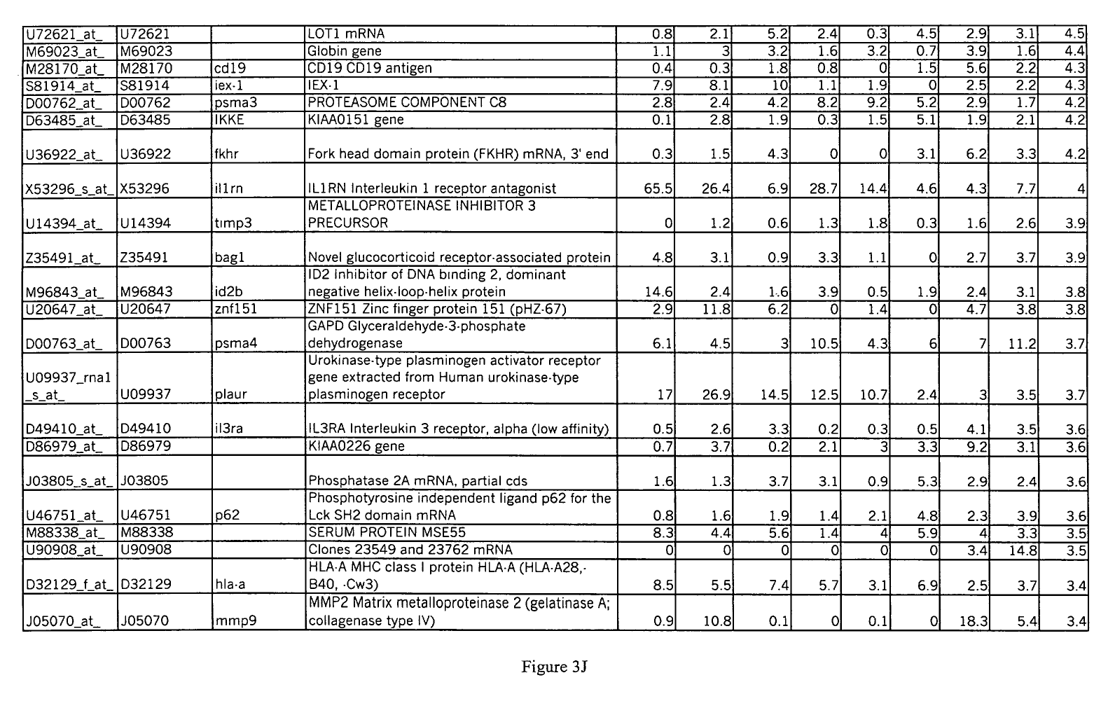

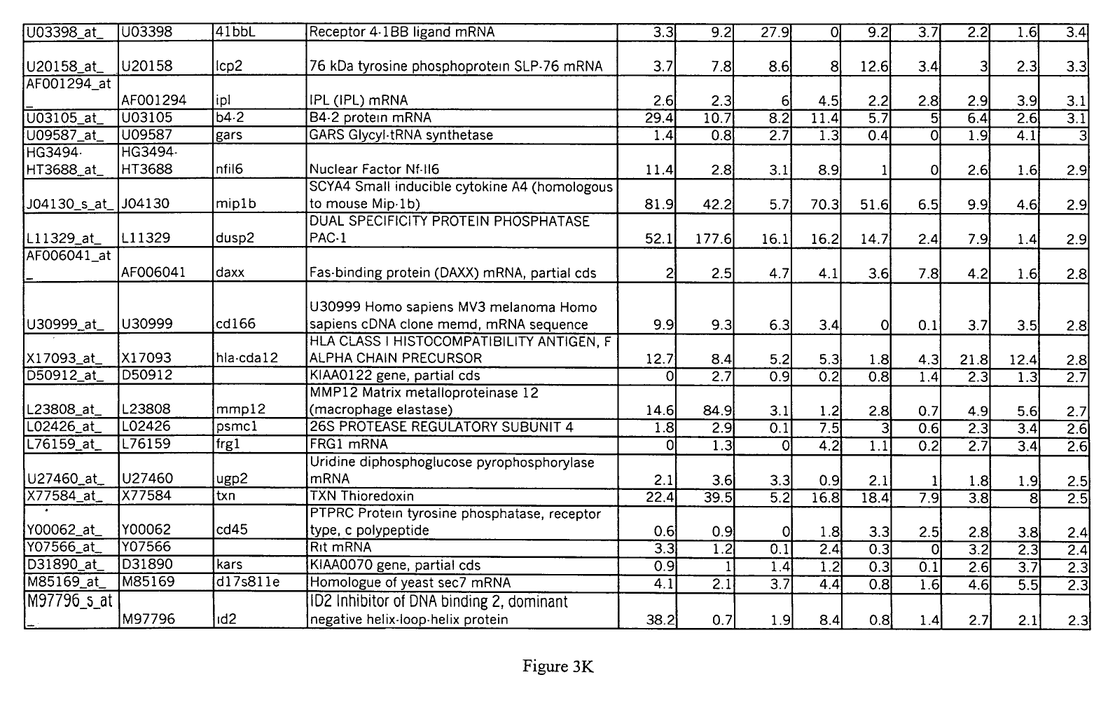

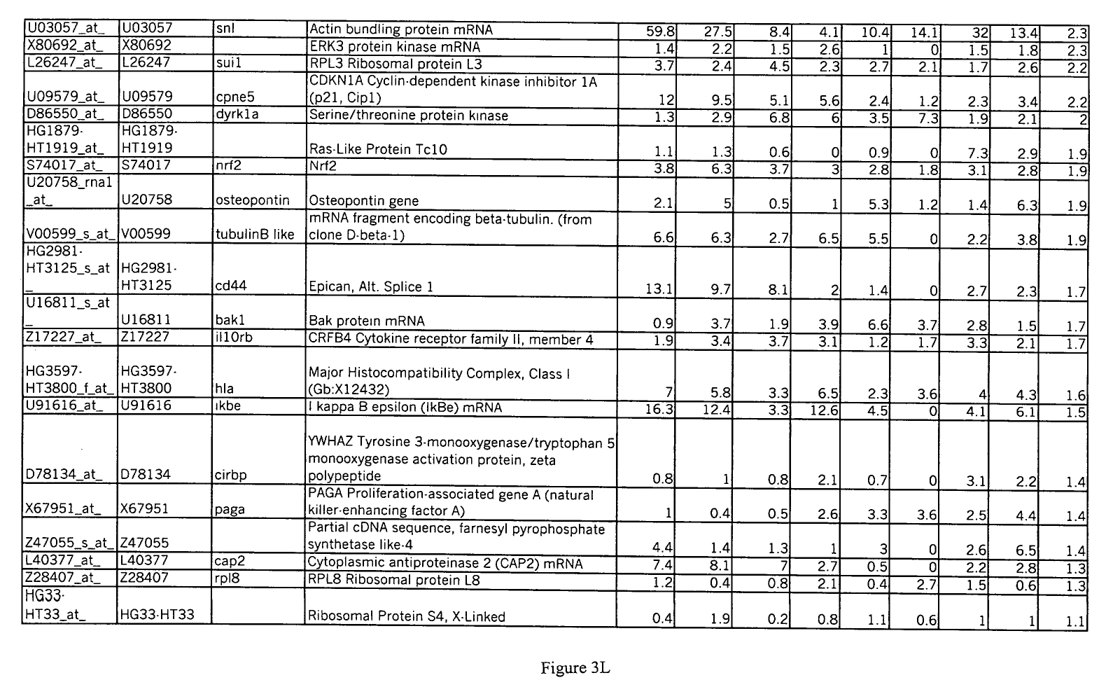

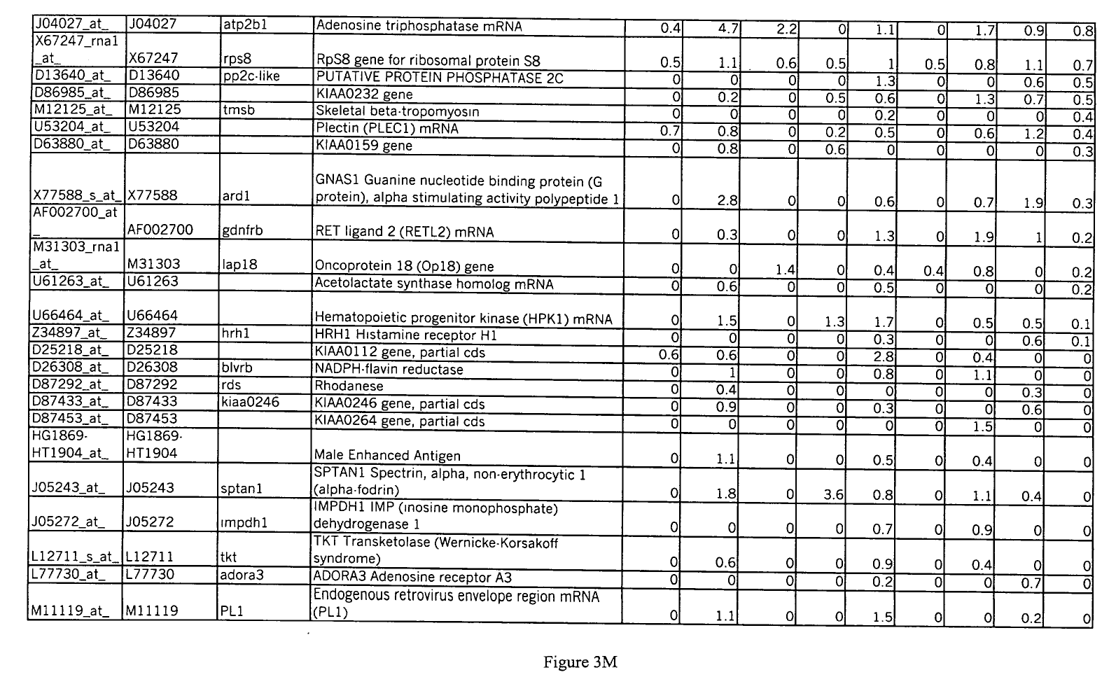

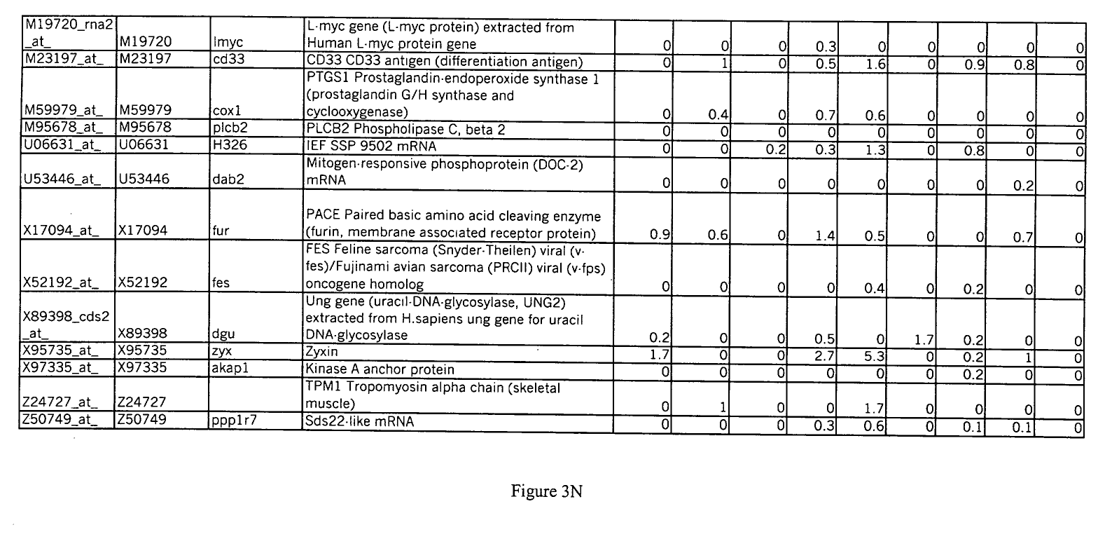

Candida: 289 genes (FIGS. 3A-3N)

Stimulus-Specific Genes

E. coli (not influenza, not Candida): 118 genes (FIGS. 4A-4F)

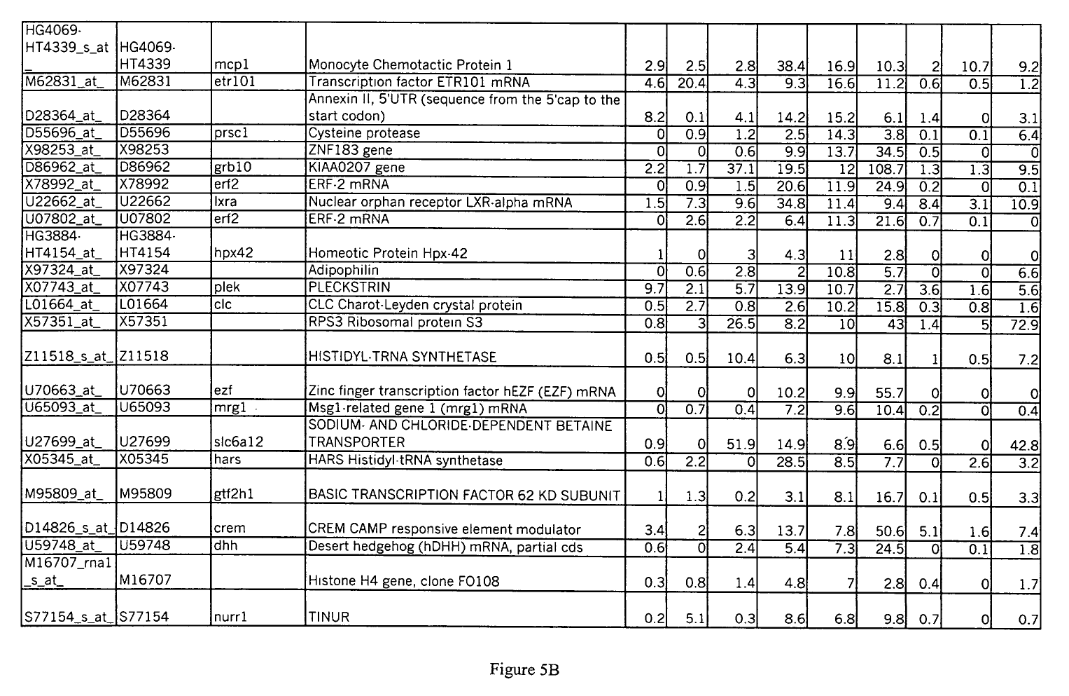

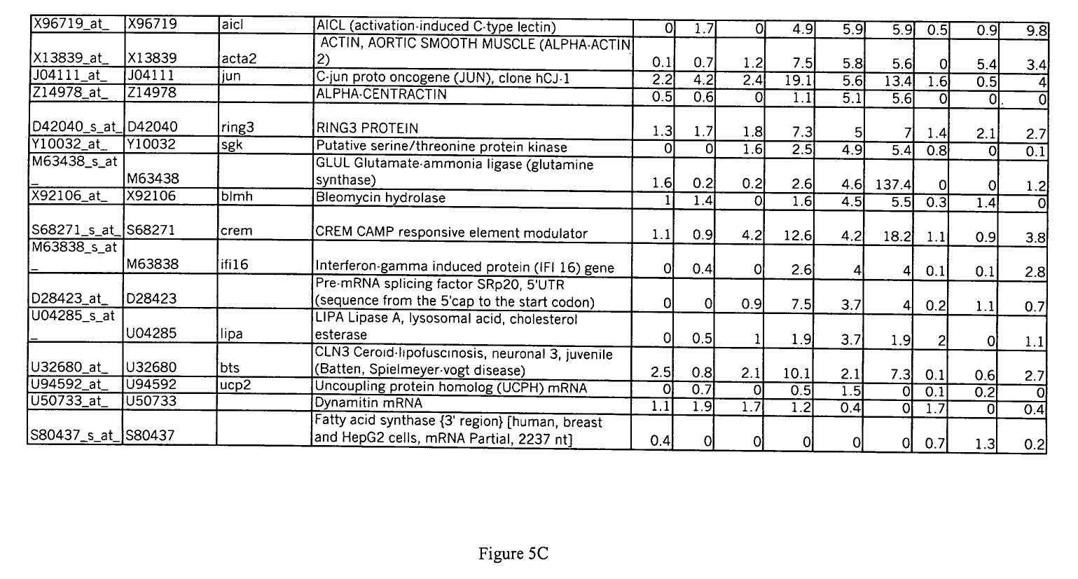

influenza (not E. coli, not Candida): 58 genes (FIGS. 5A-5C)

Candida (not E. coli, not influenza): 0 genes

Common Stimulus-Responsive Genes

FIGS. 6A-6E, 165 common stimulus-responsive genes were identified. A schematic representation of common stimulus-responsive genes is shown in FIG. 7A.

A list of the different types or classes of genes that respond to the stimuli are shown in FIGS. 10A-10C.

### EXAMPLE 3

### Identification of Pathogen Regulated Genes Using Affymetrix GeneChip® Human Genome U95 Set (60,000 Genes) (1 Donor)

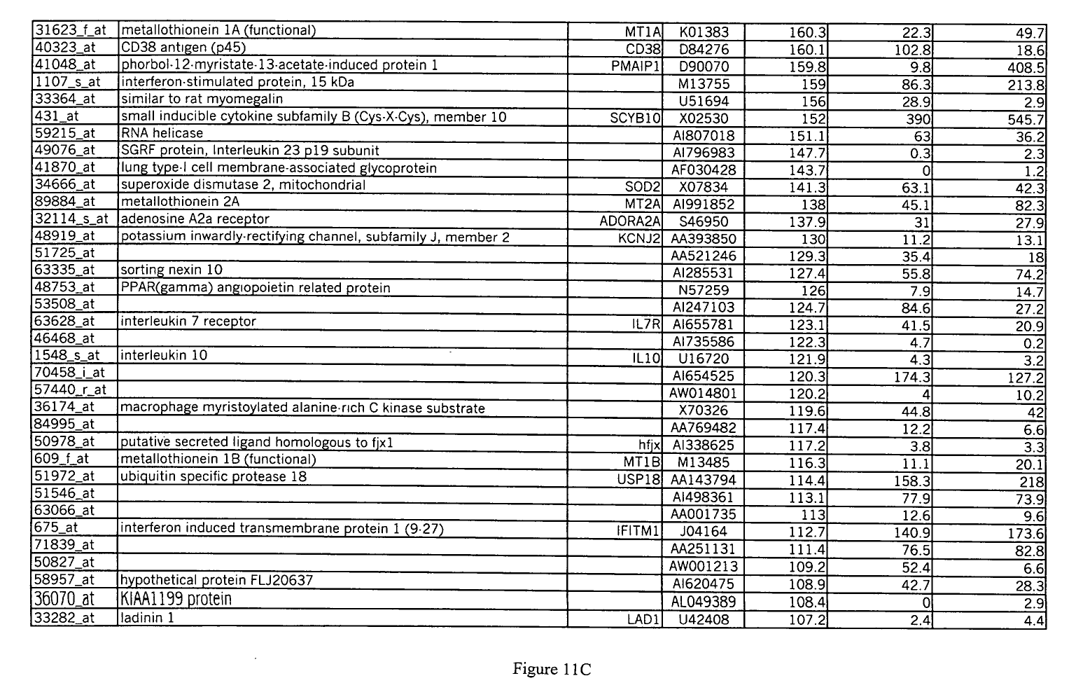

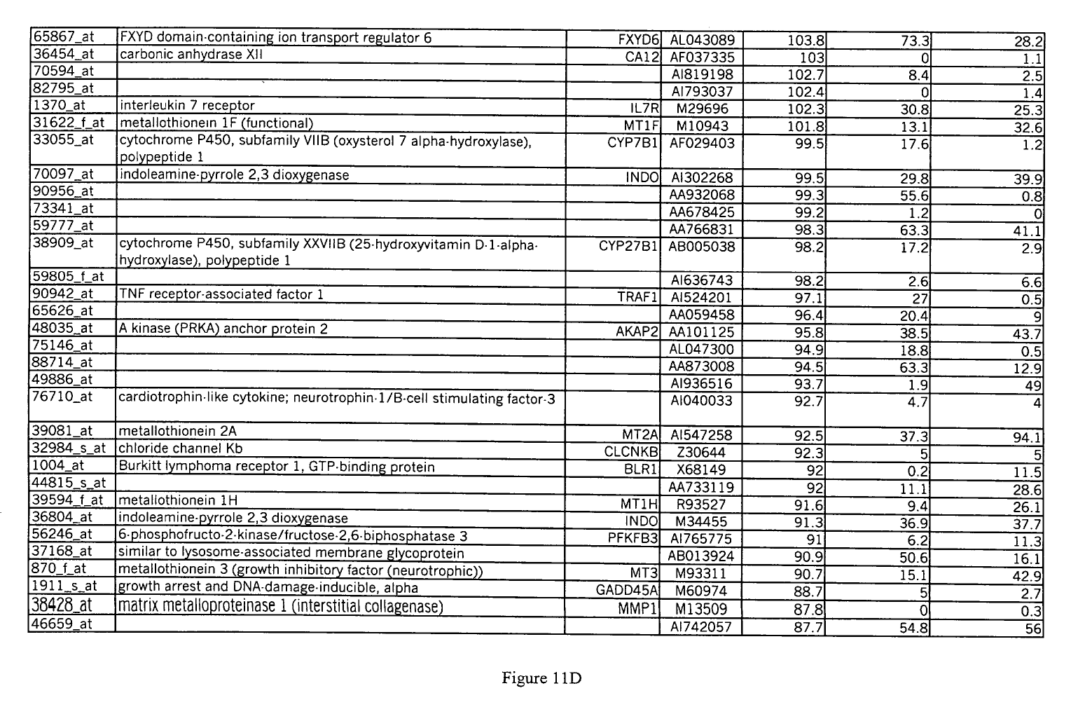

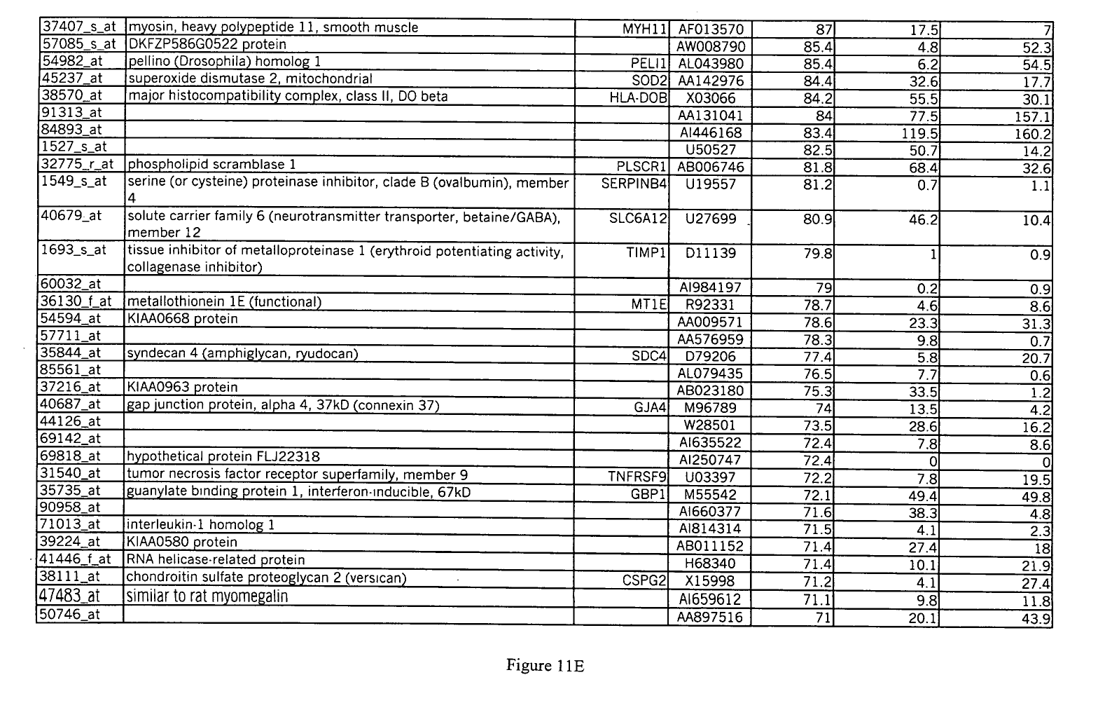

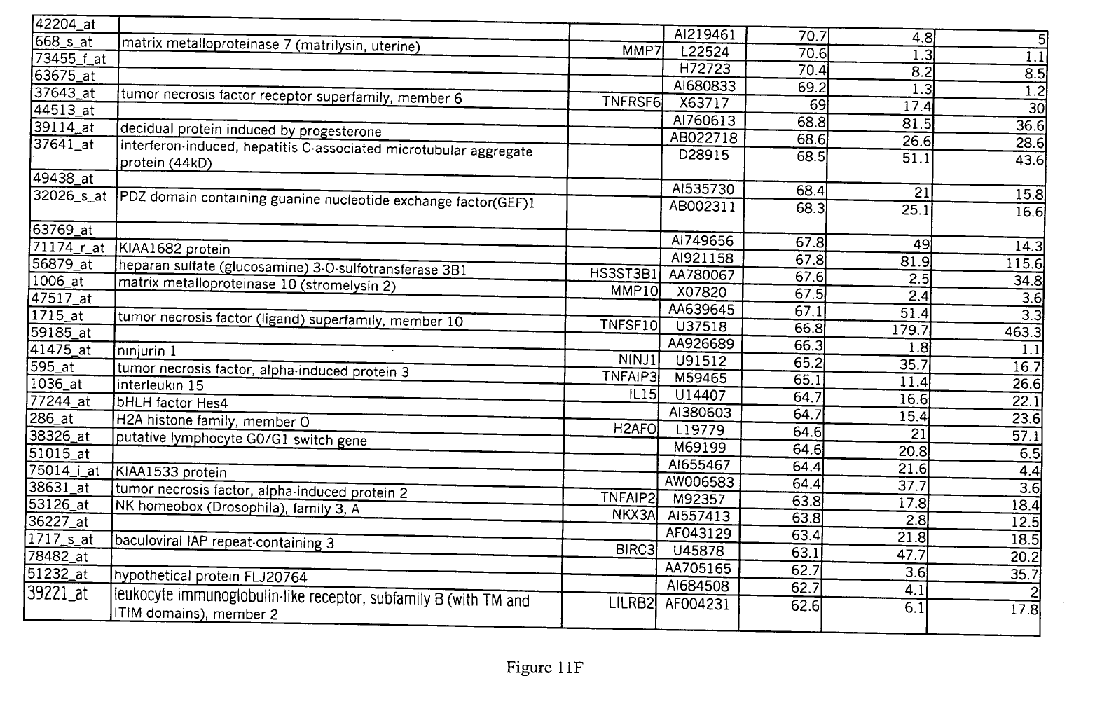

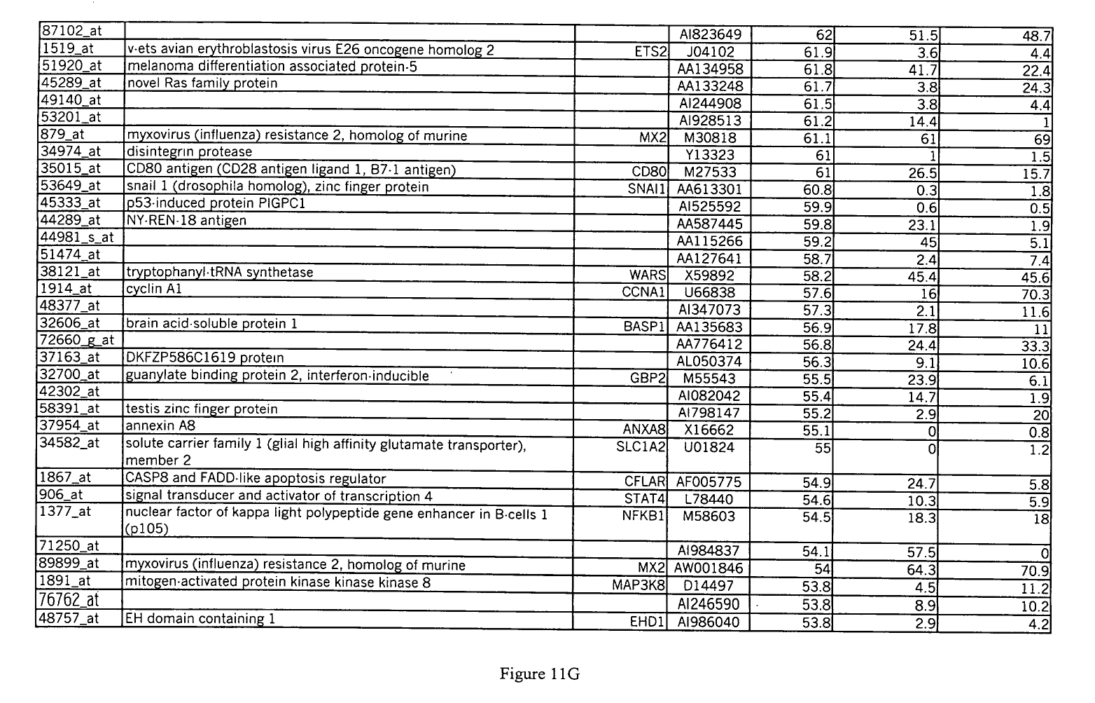

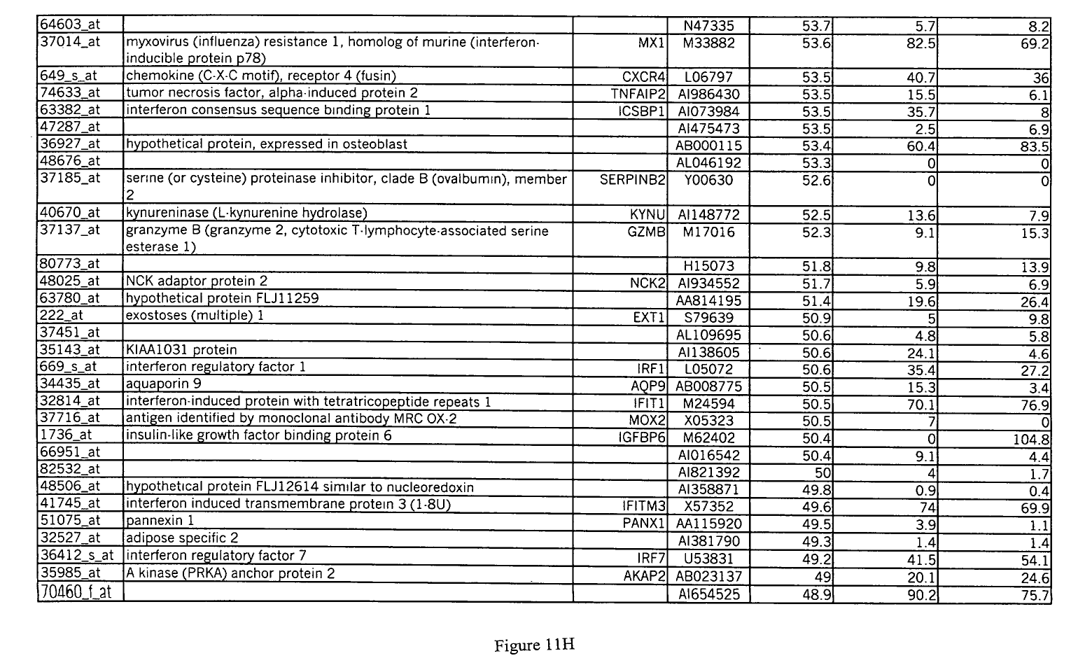

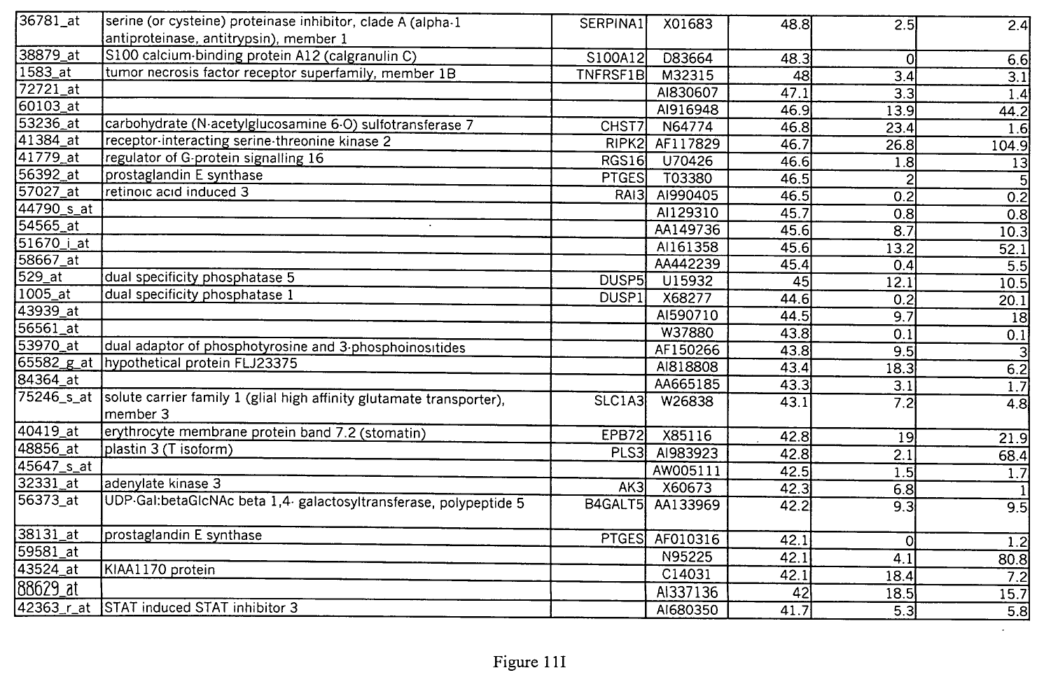

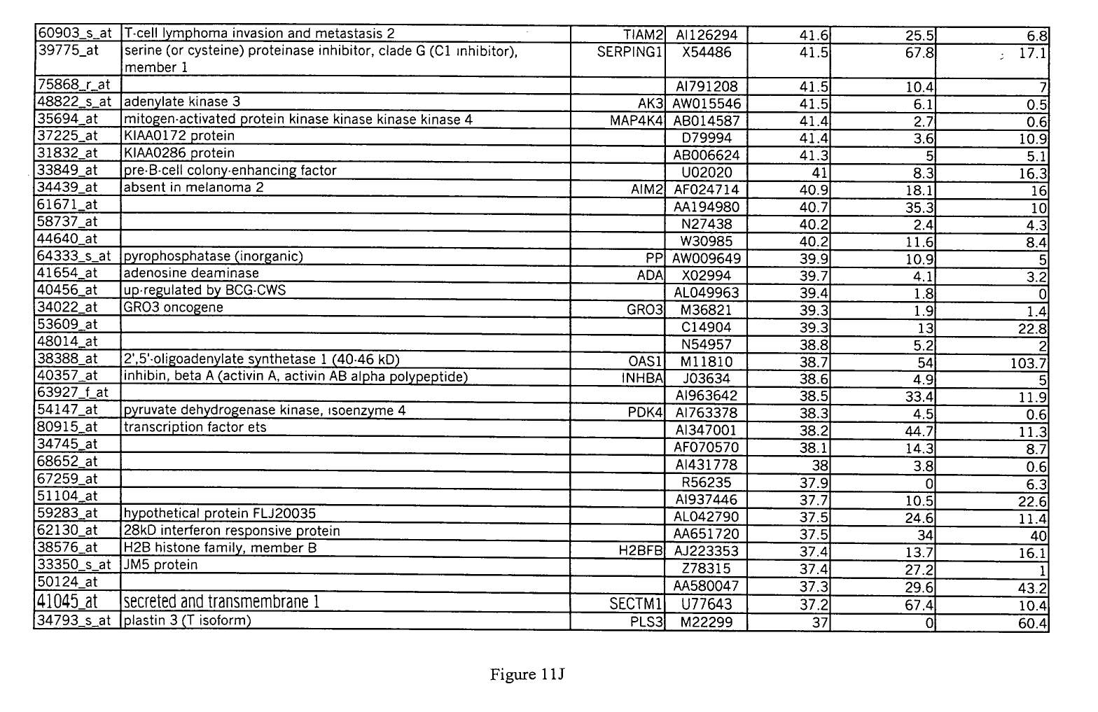

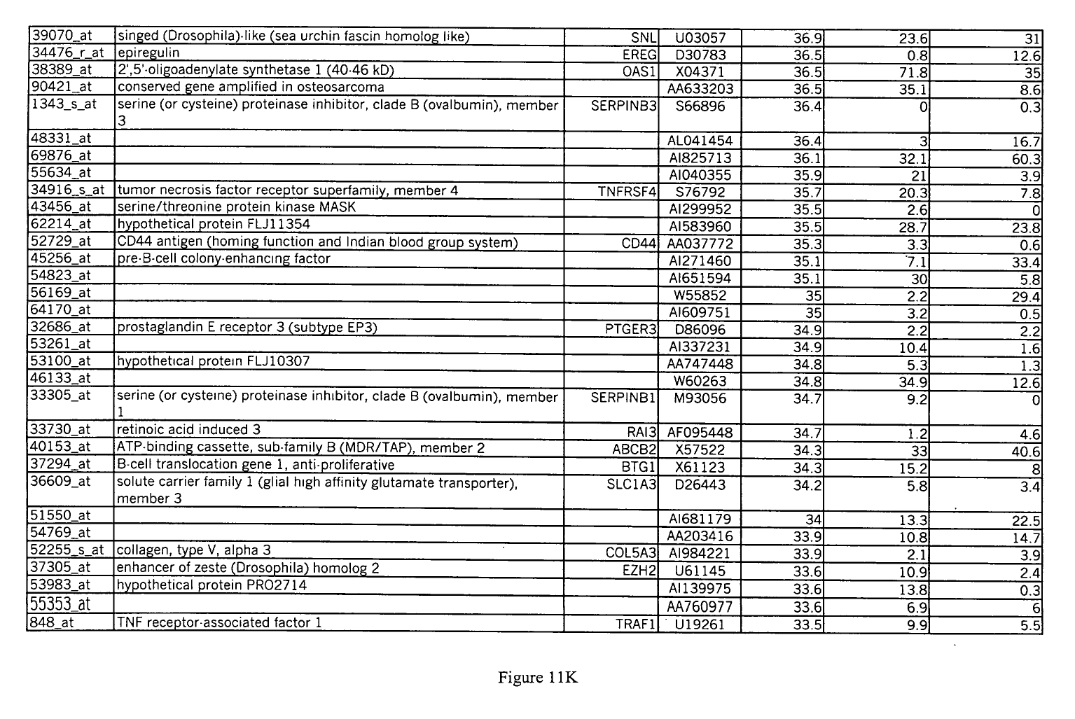

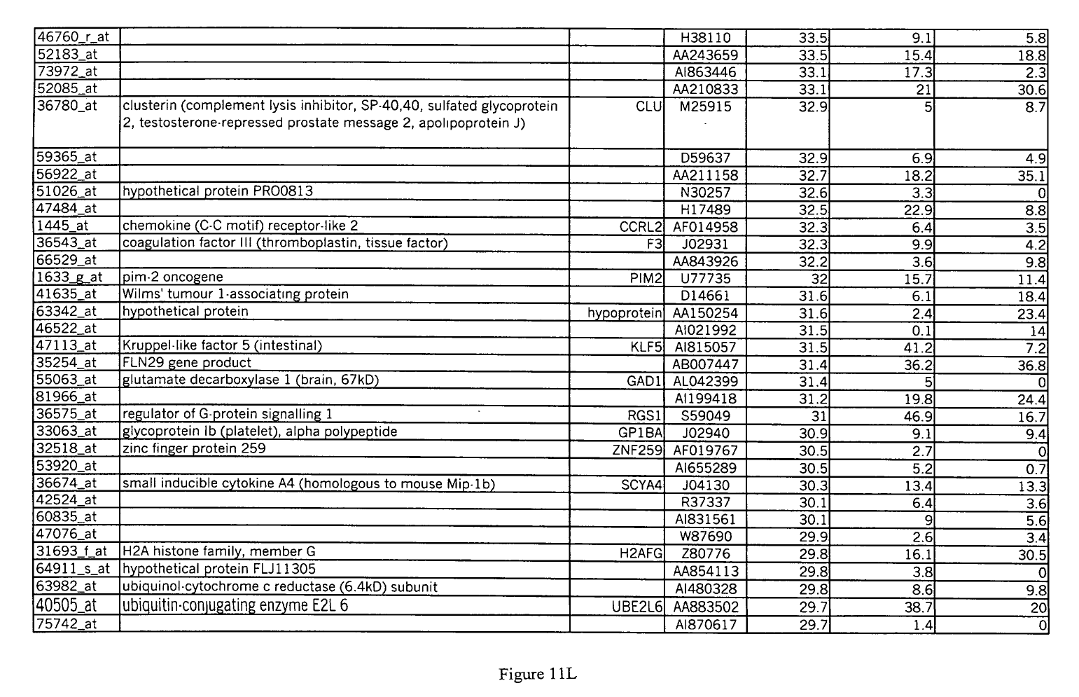

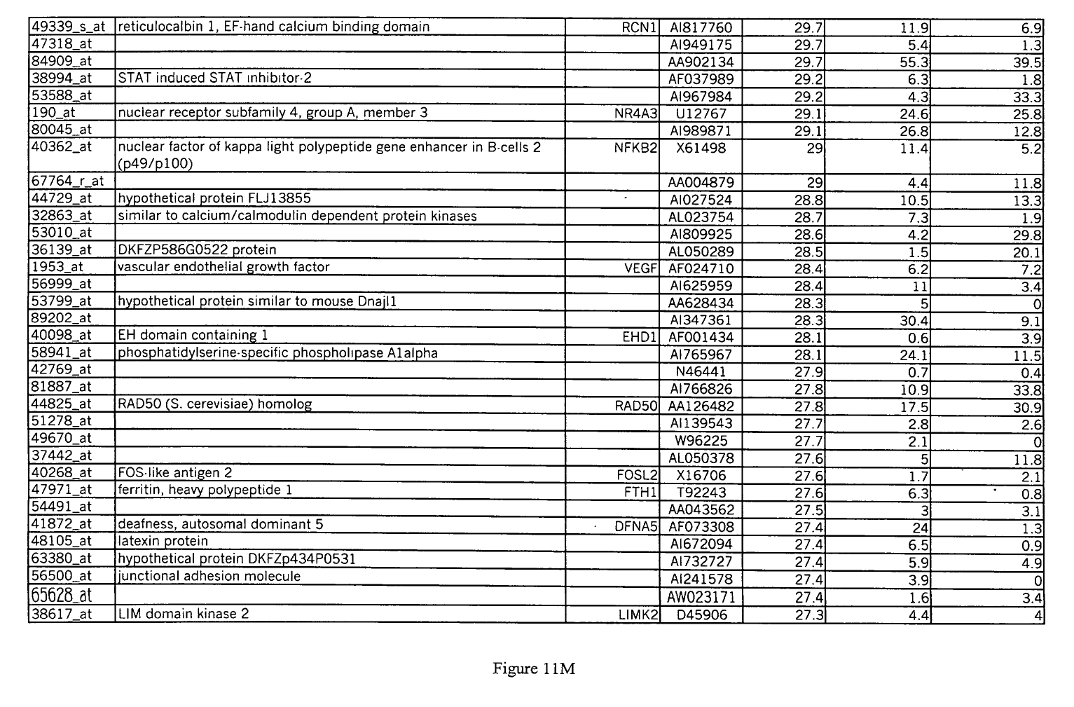

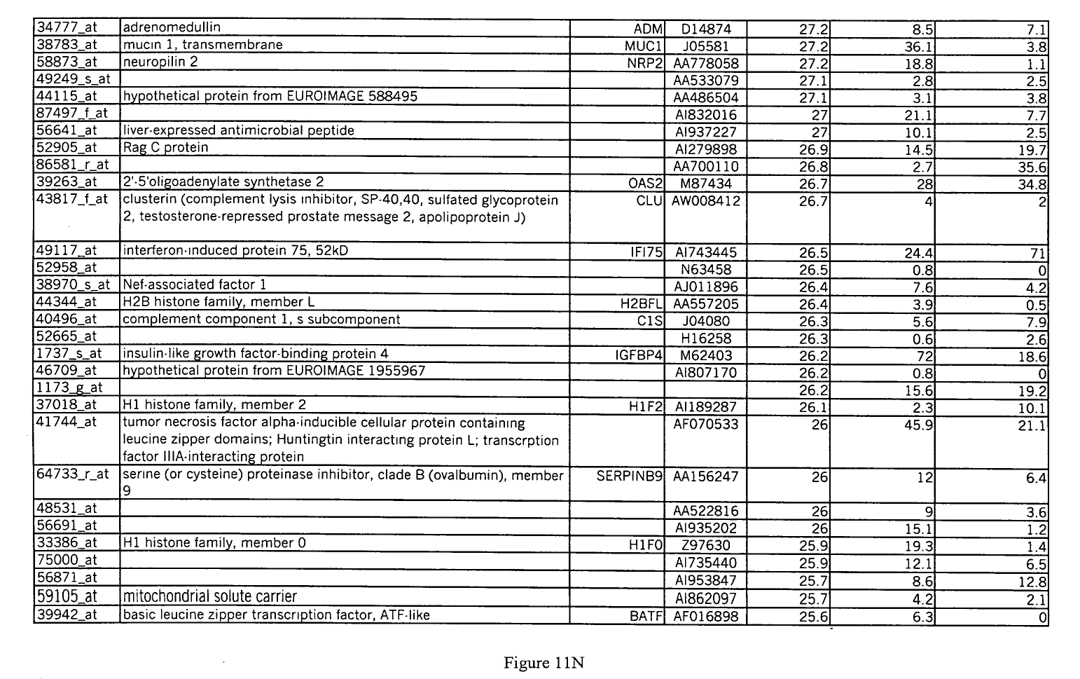

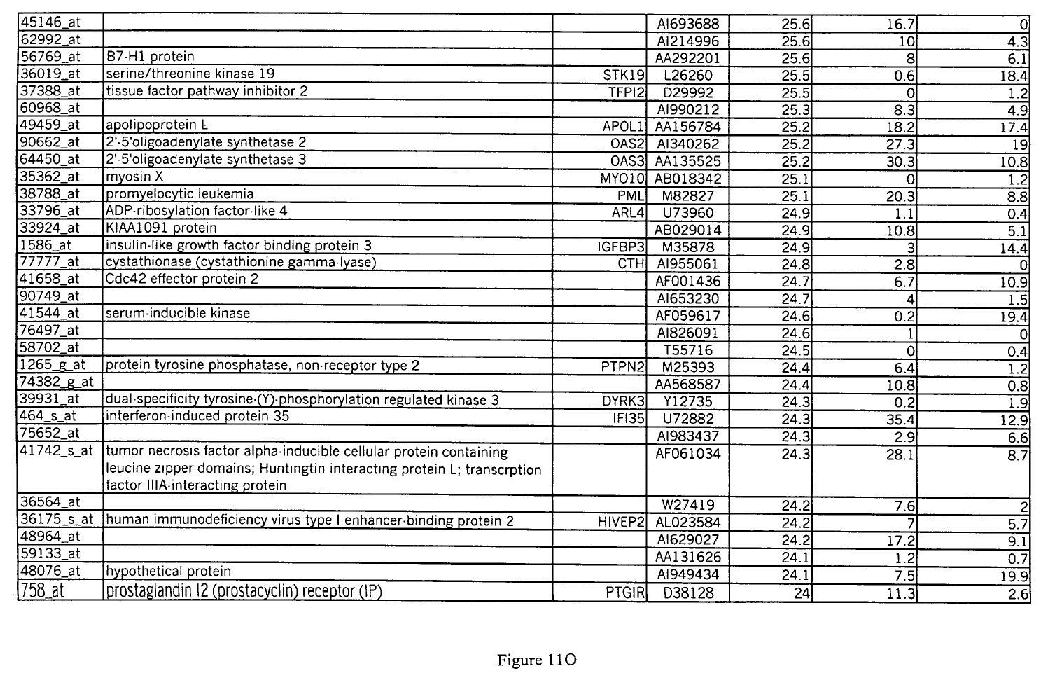

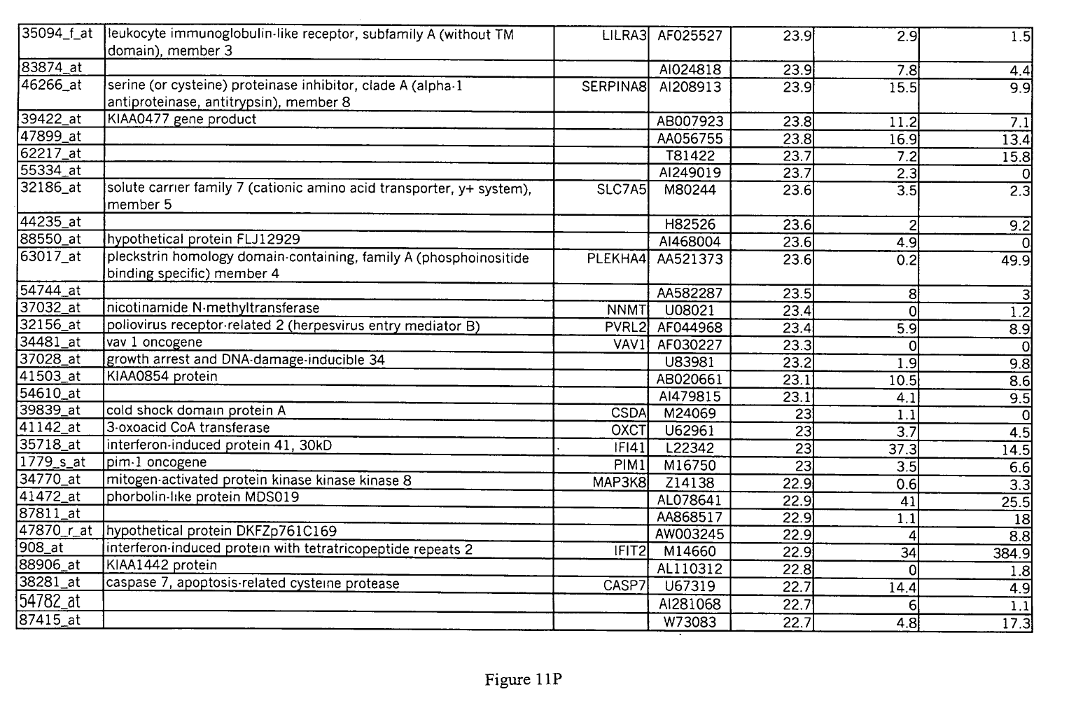

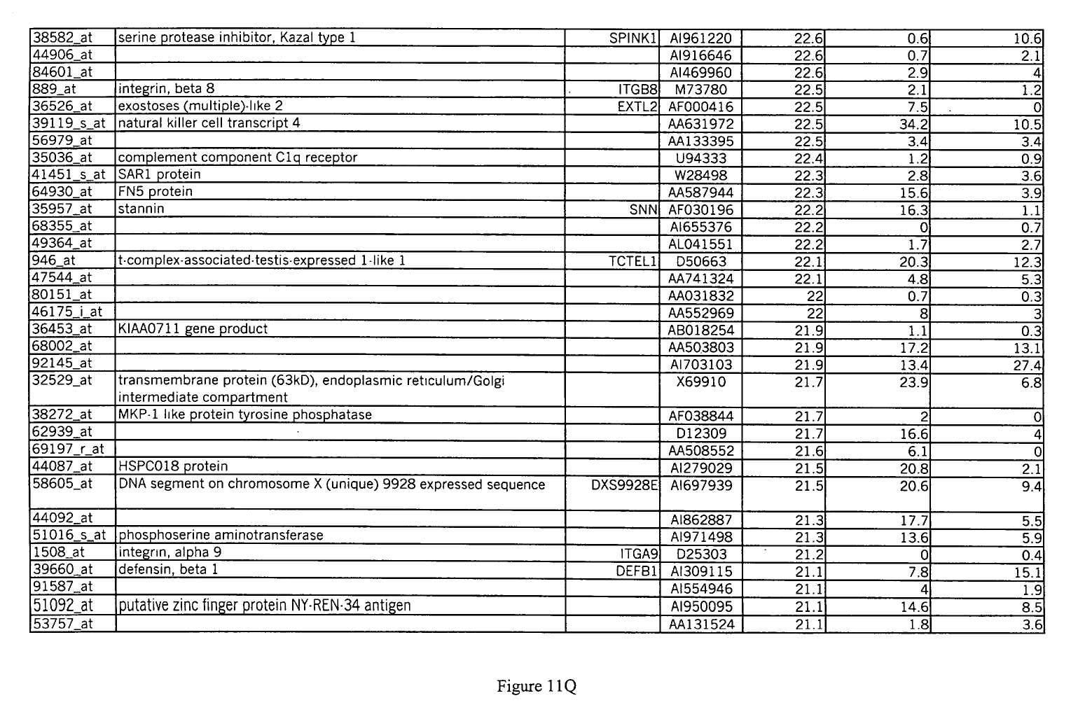

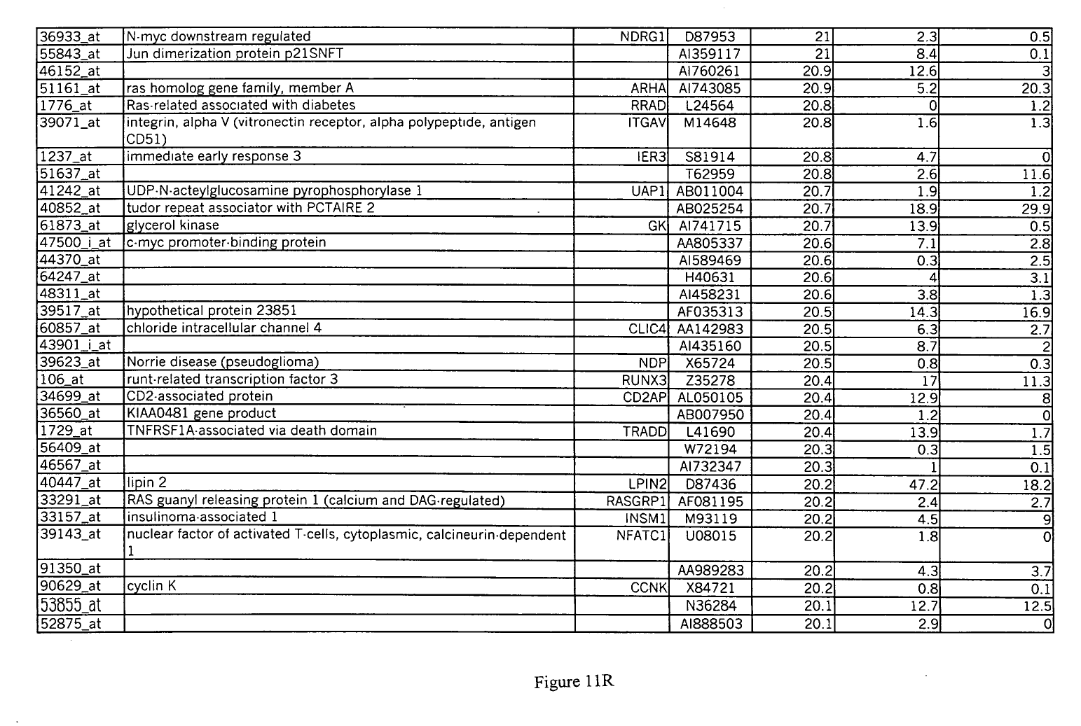

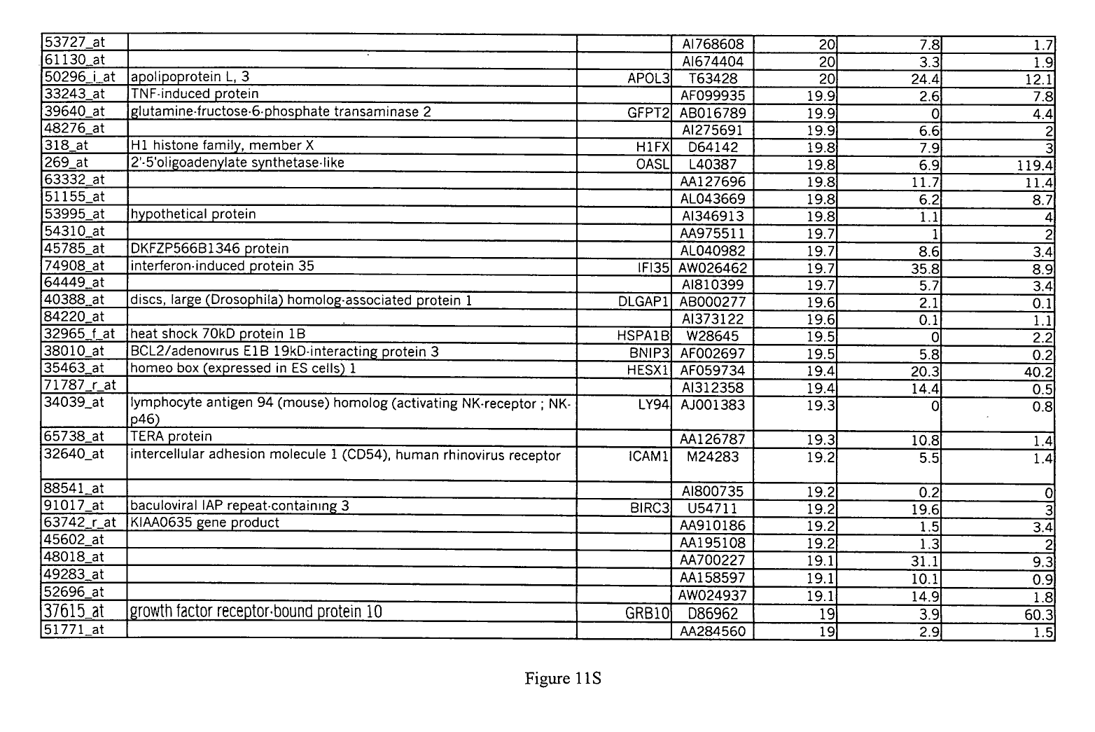

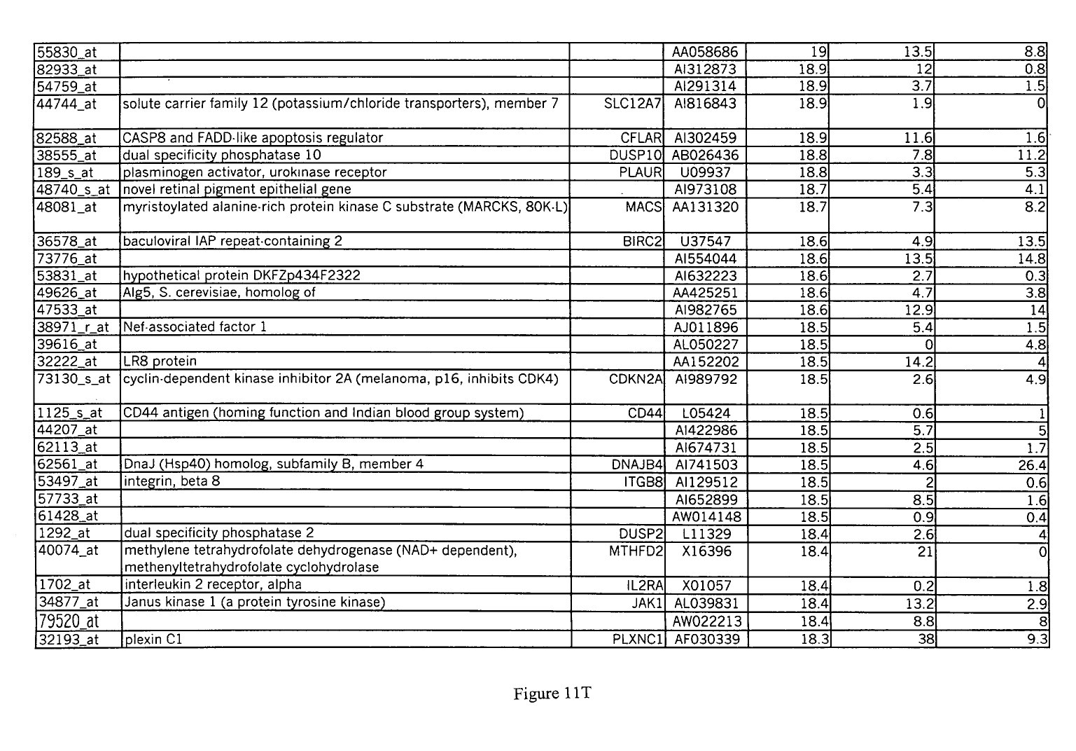

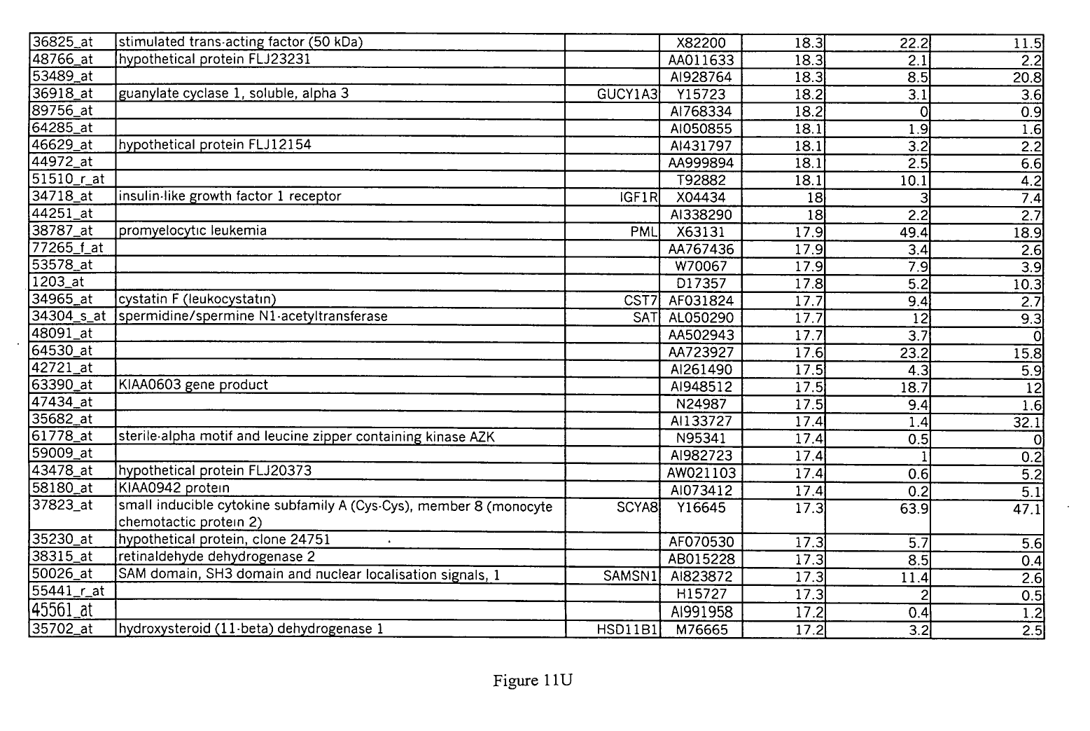

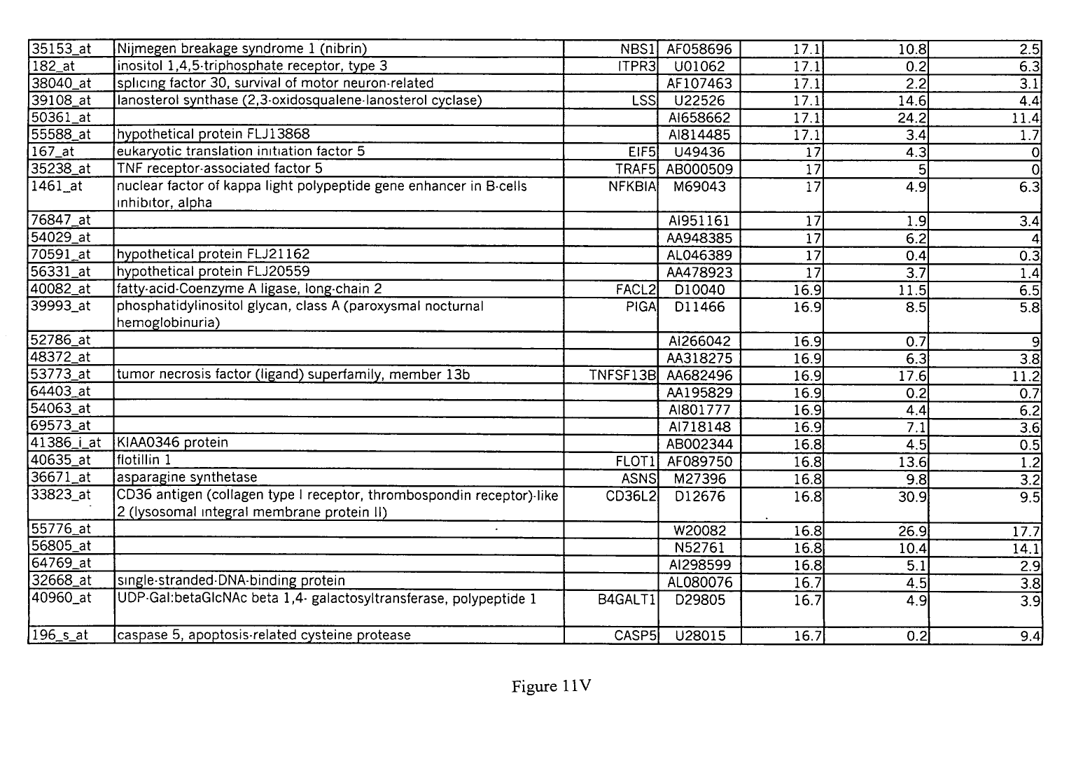

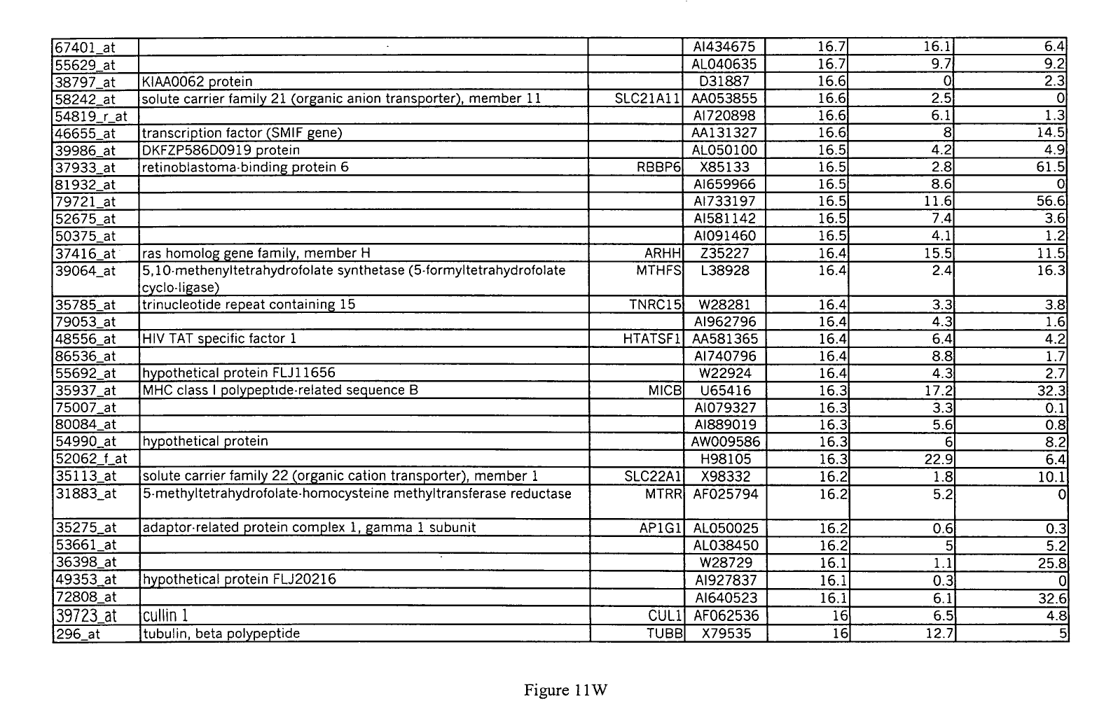

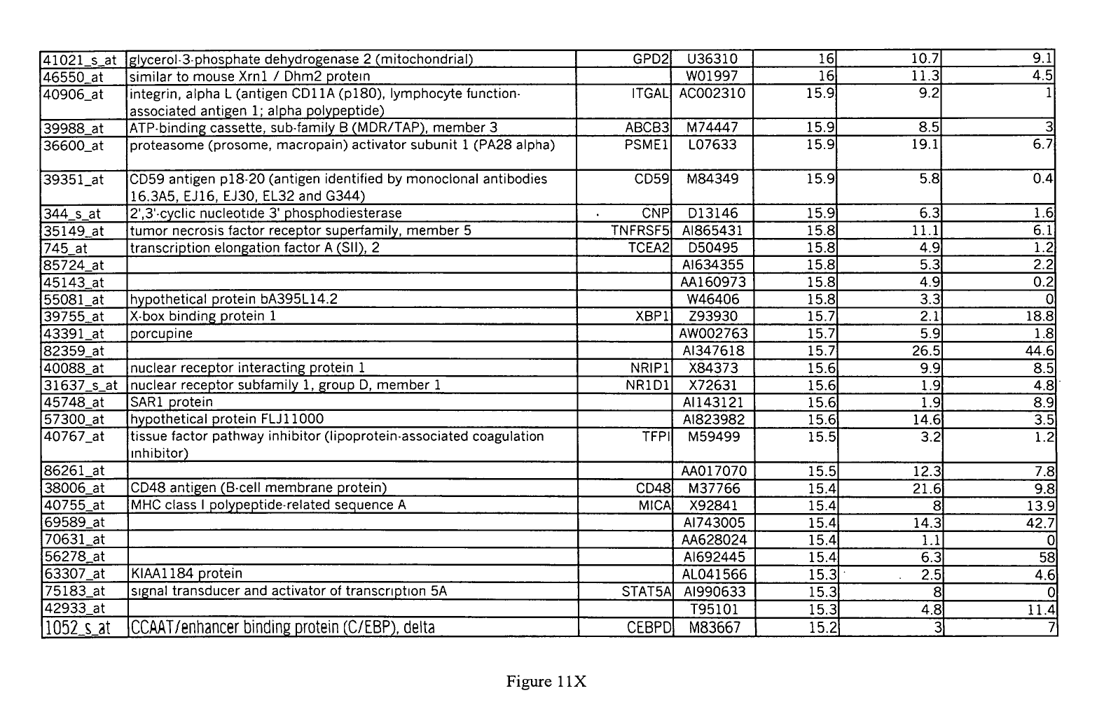

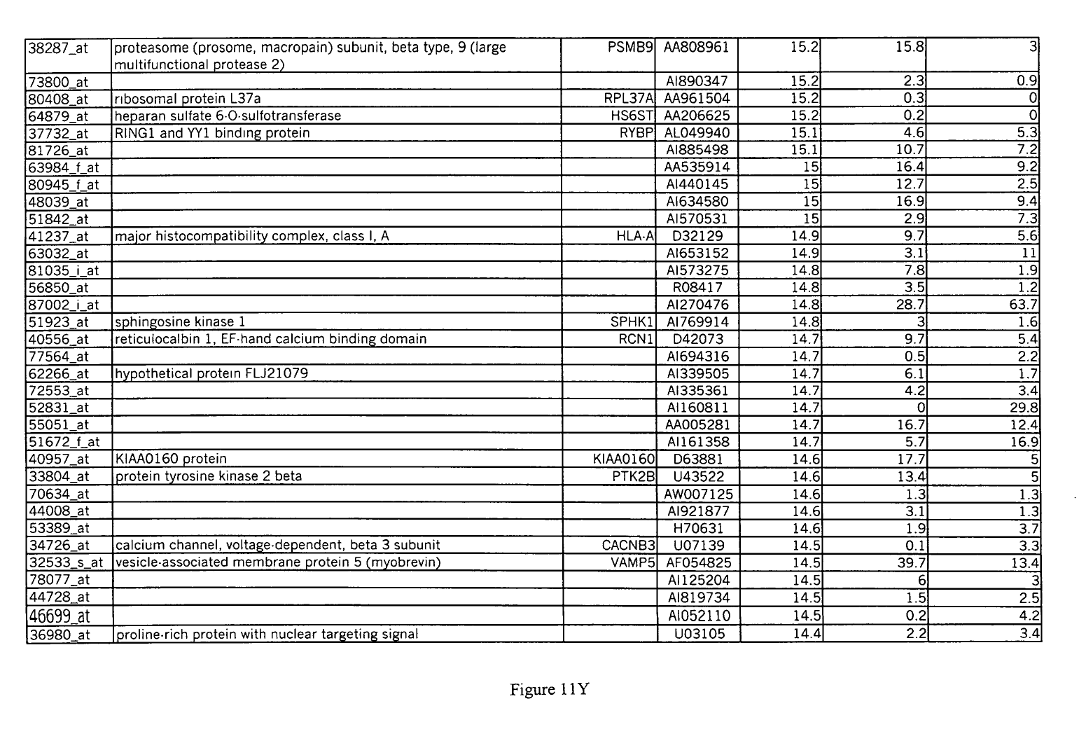

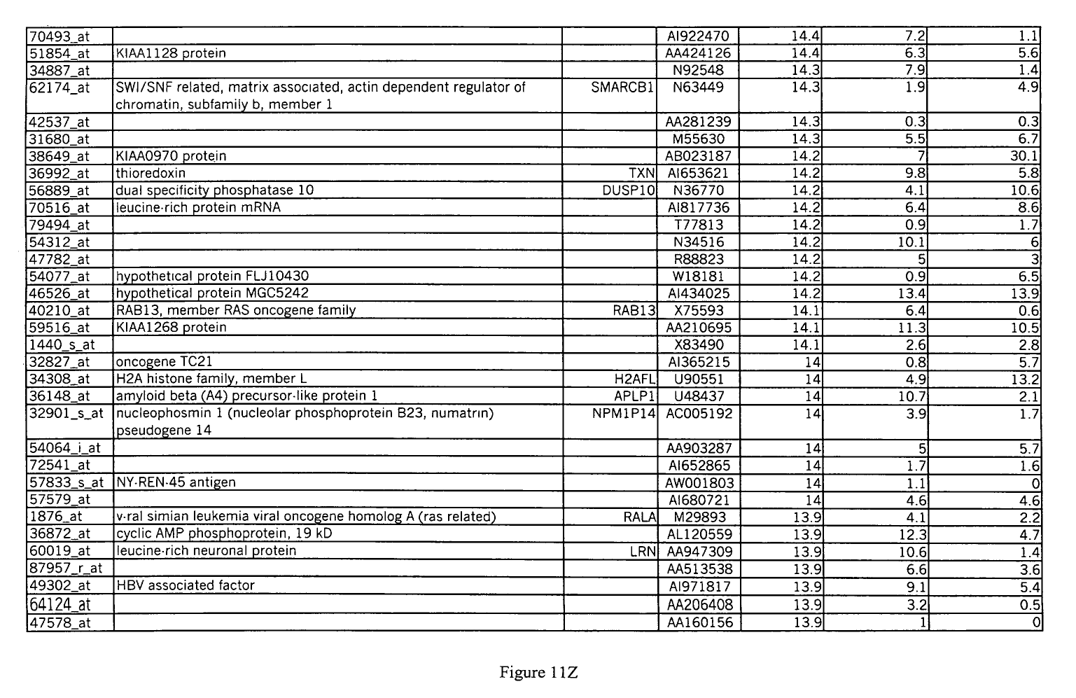

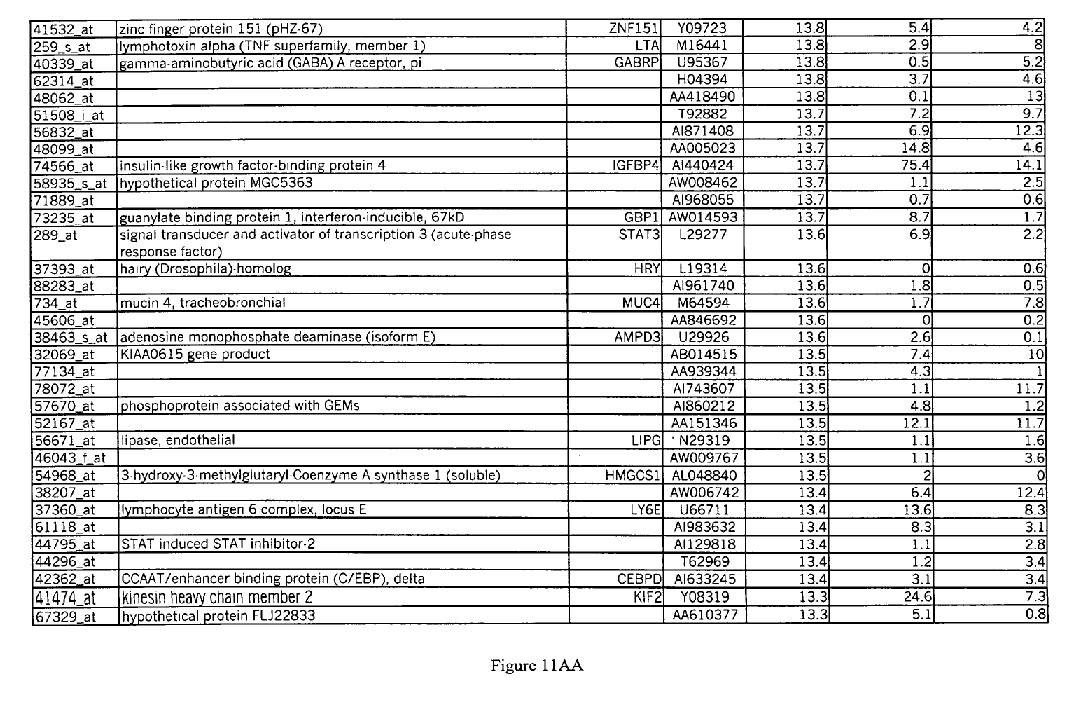

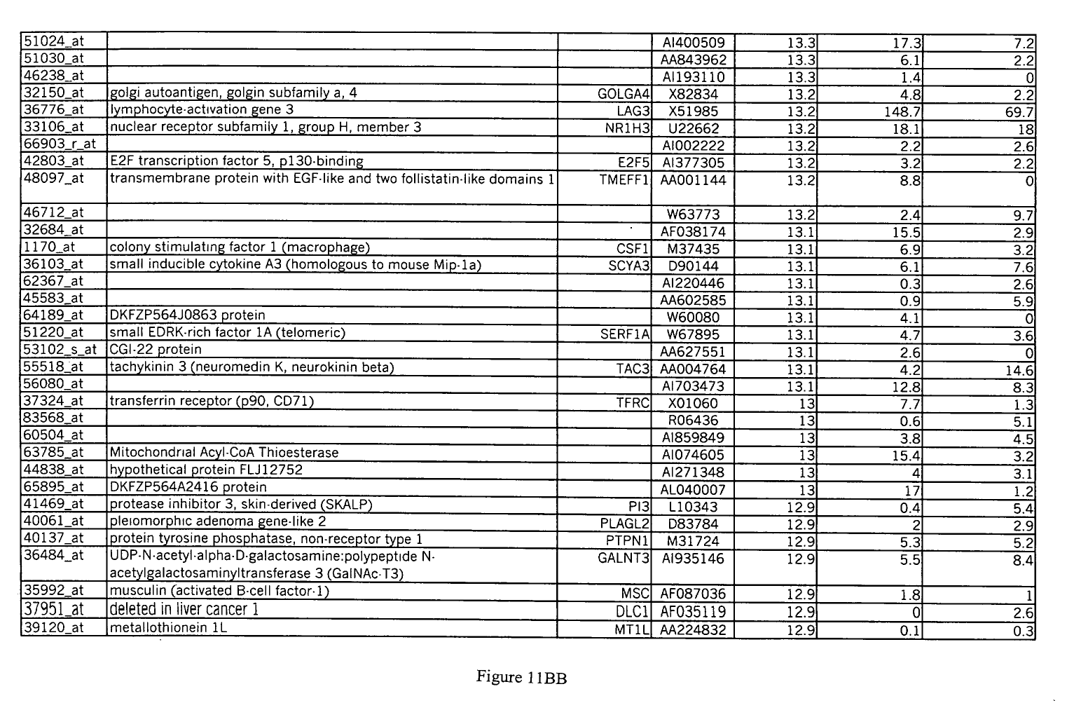

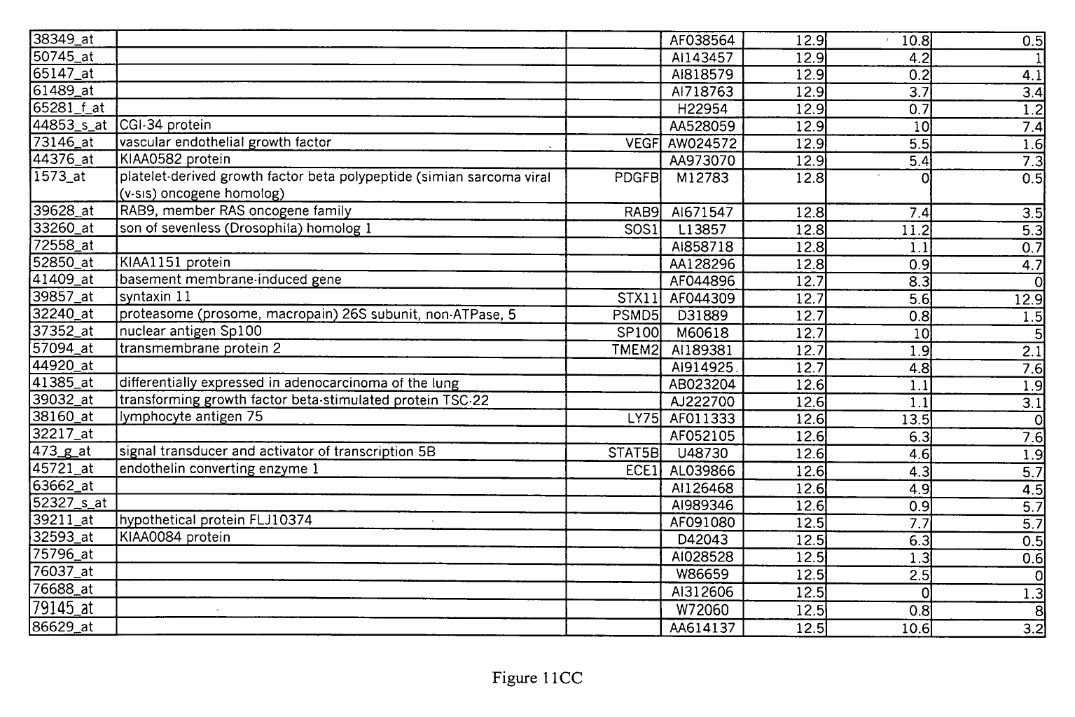

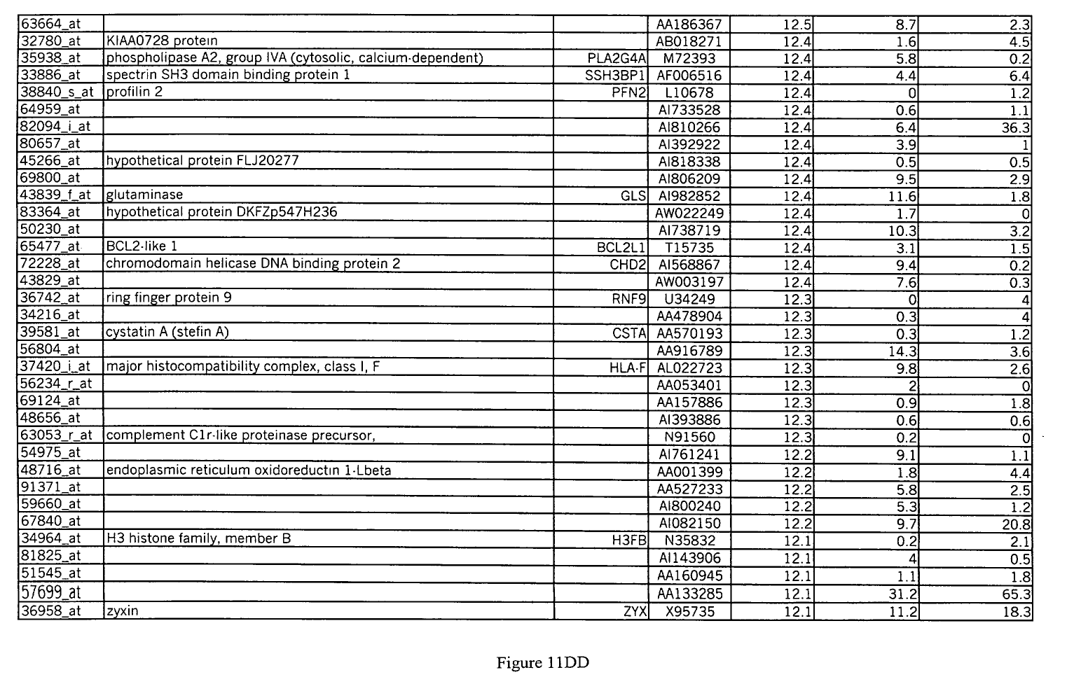

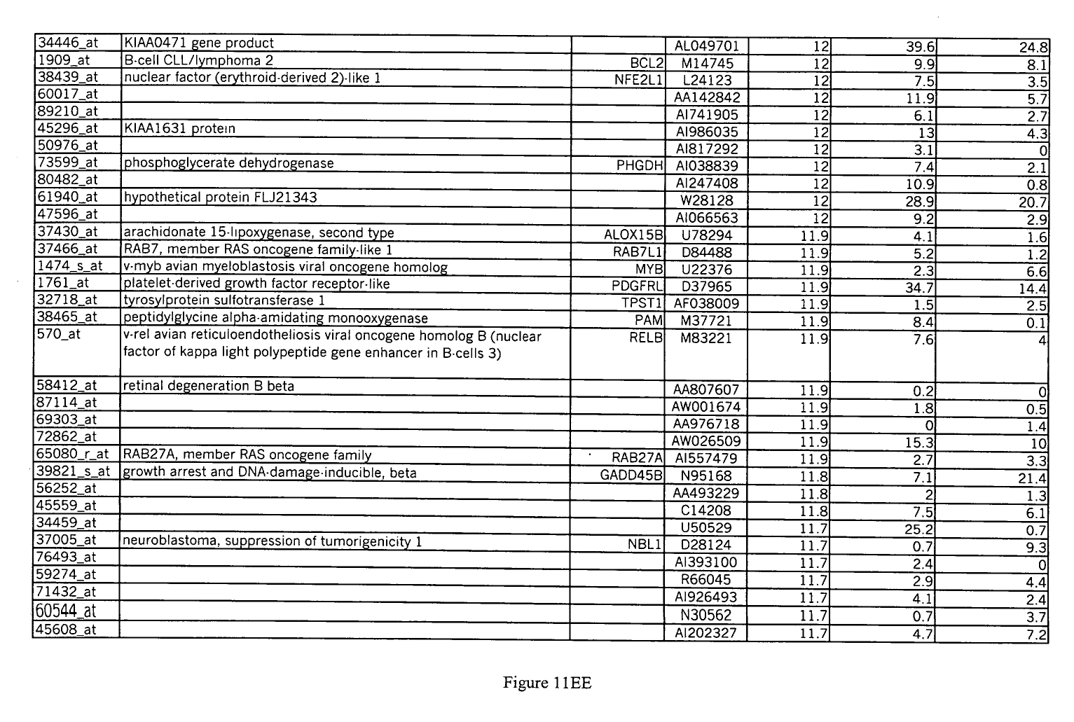

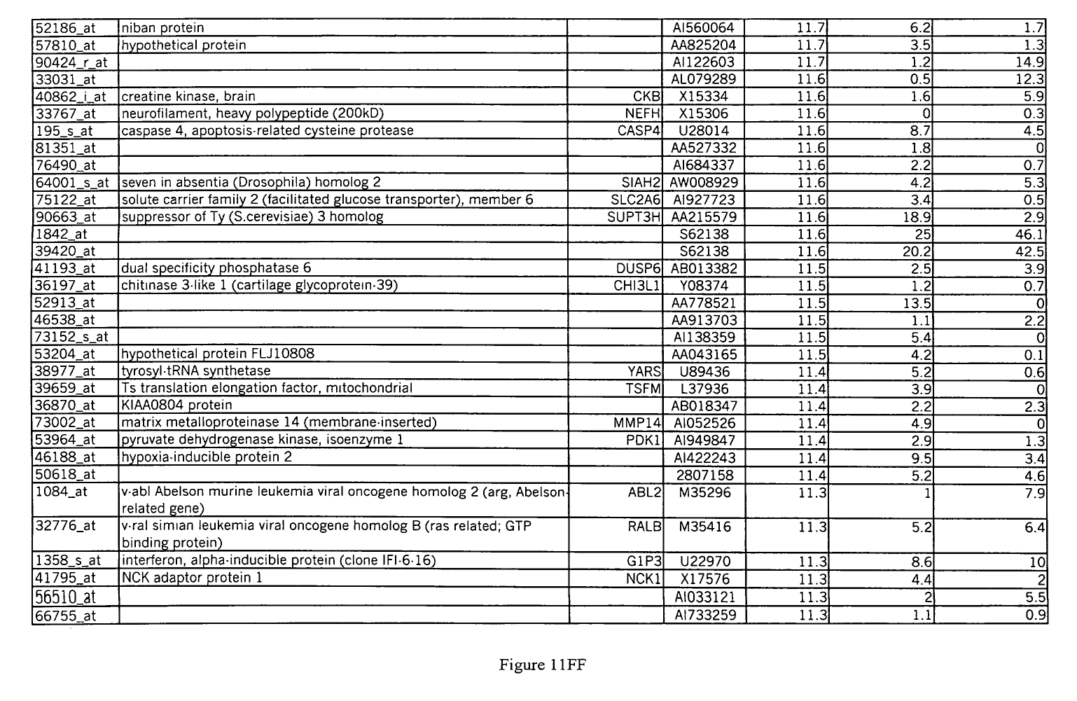

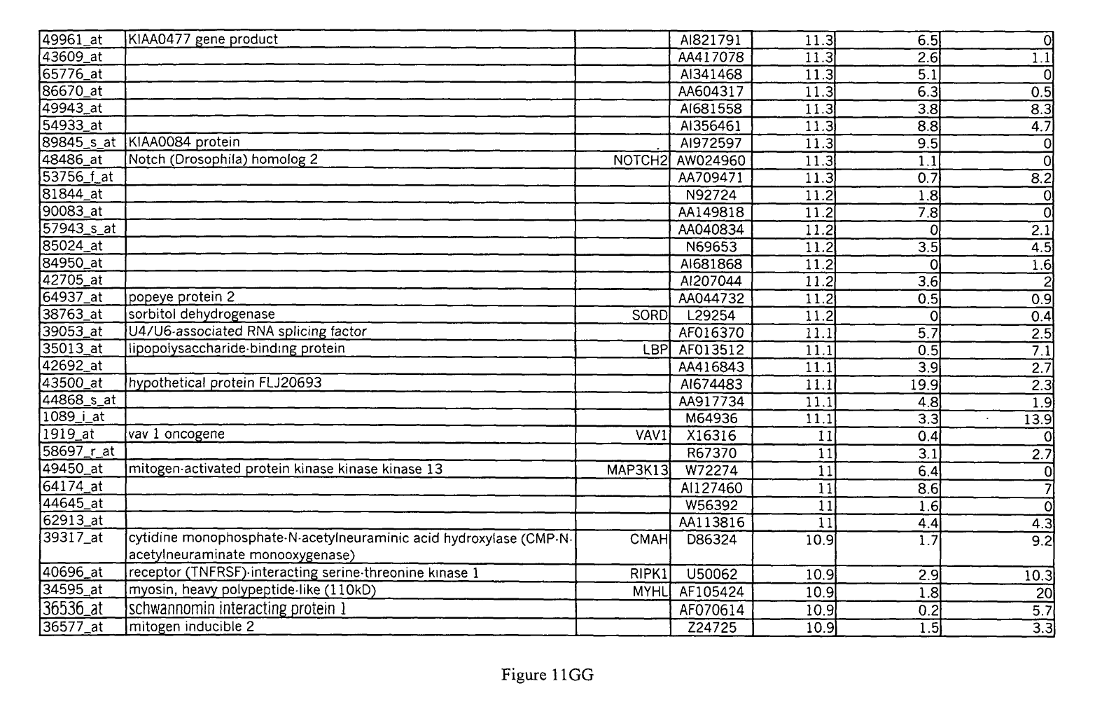

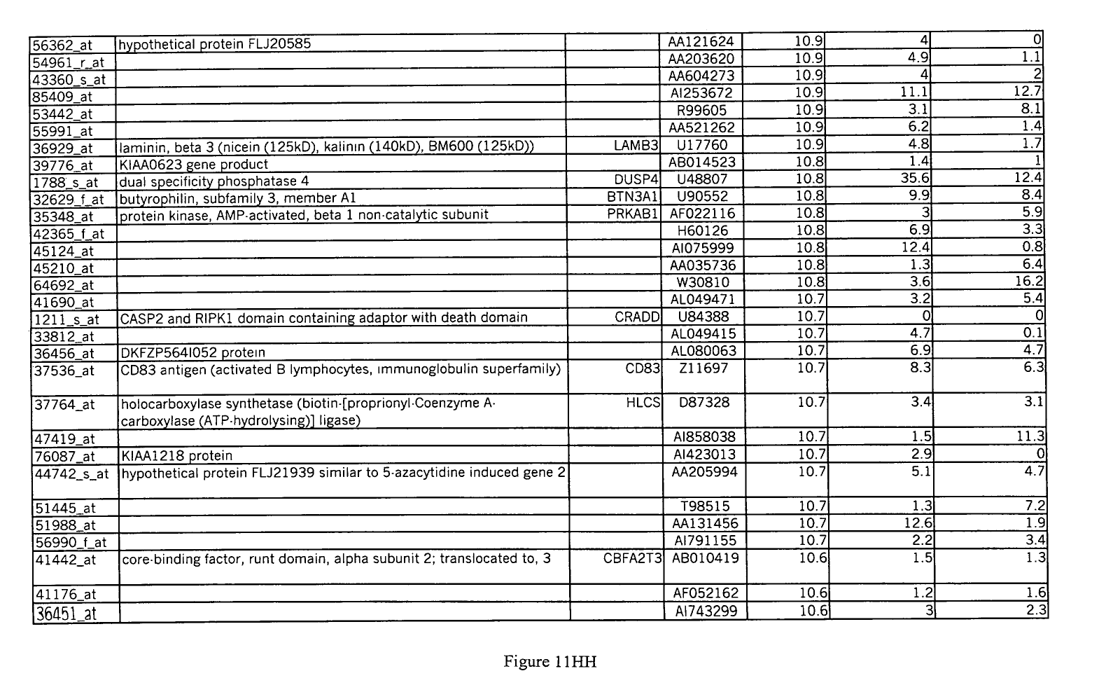

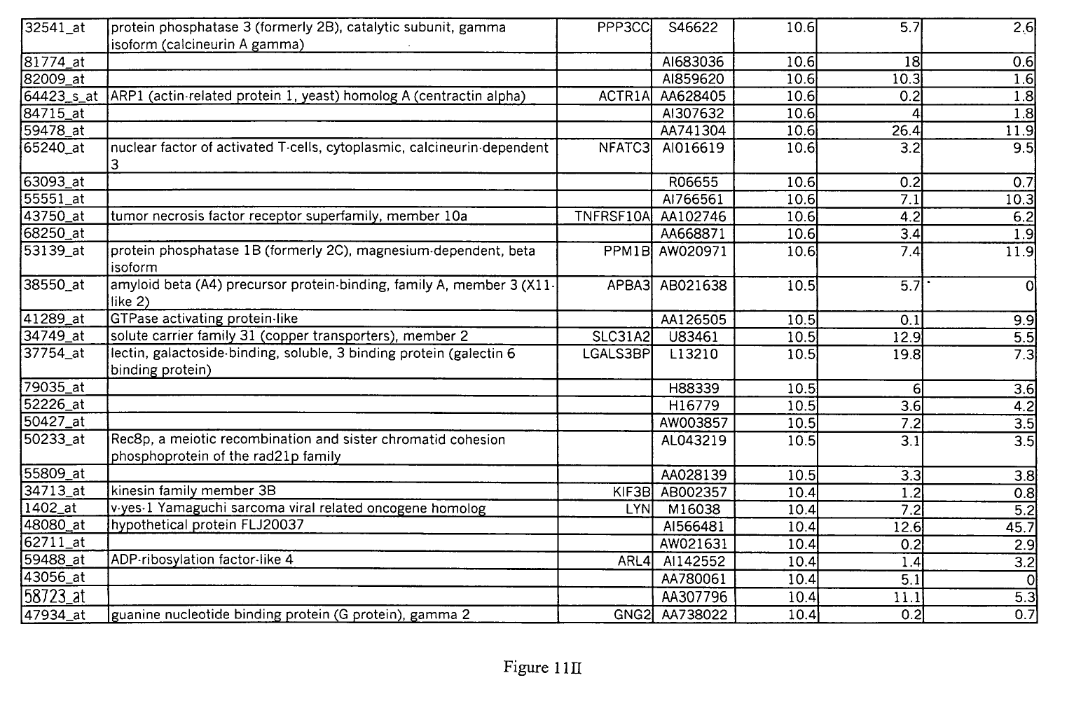

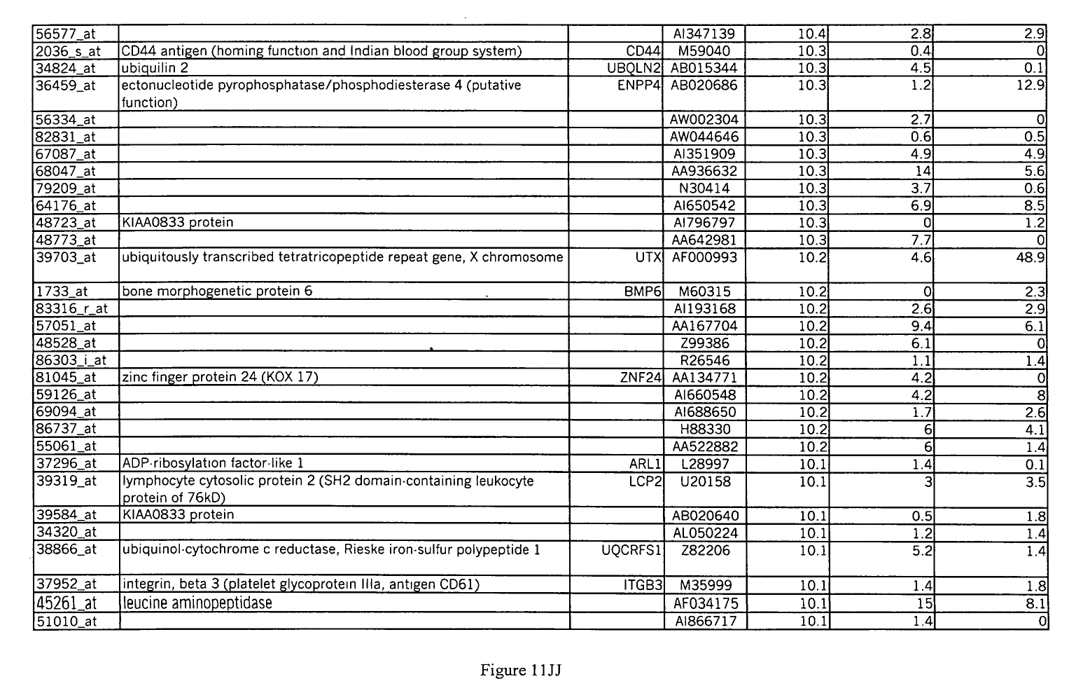

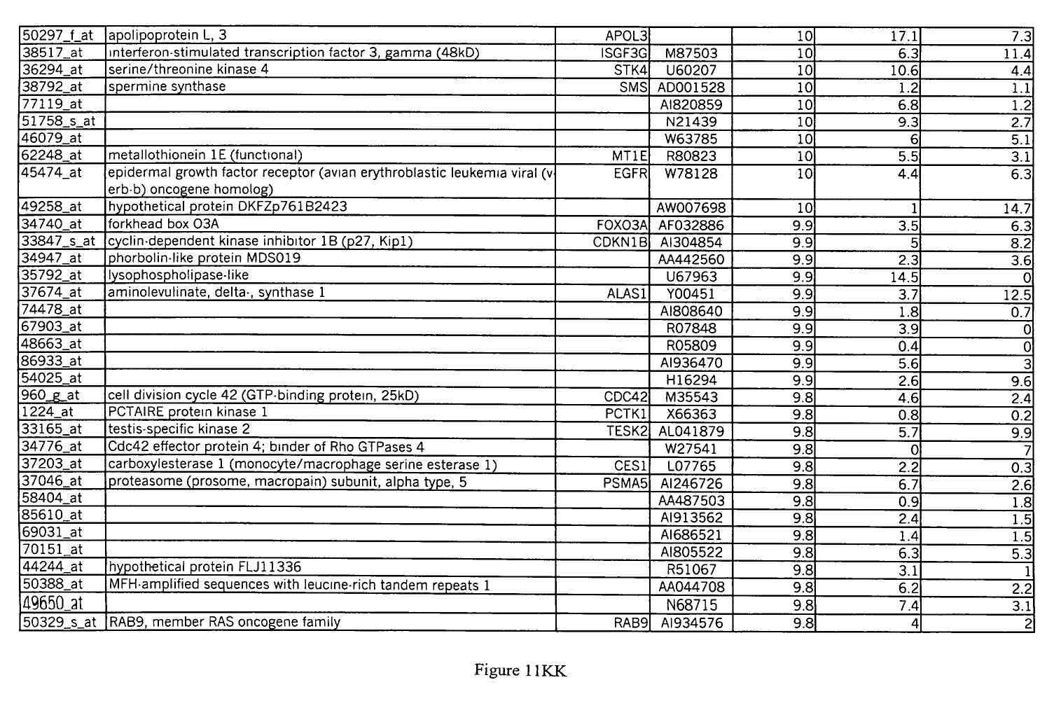

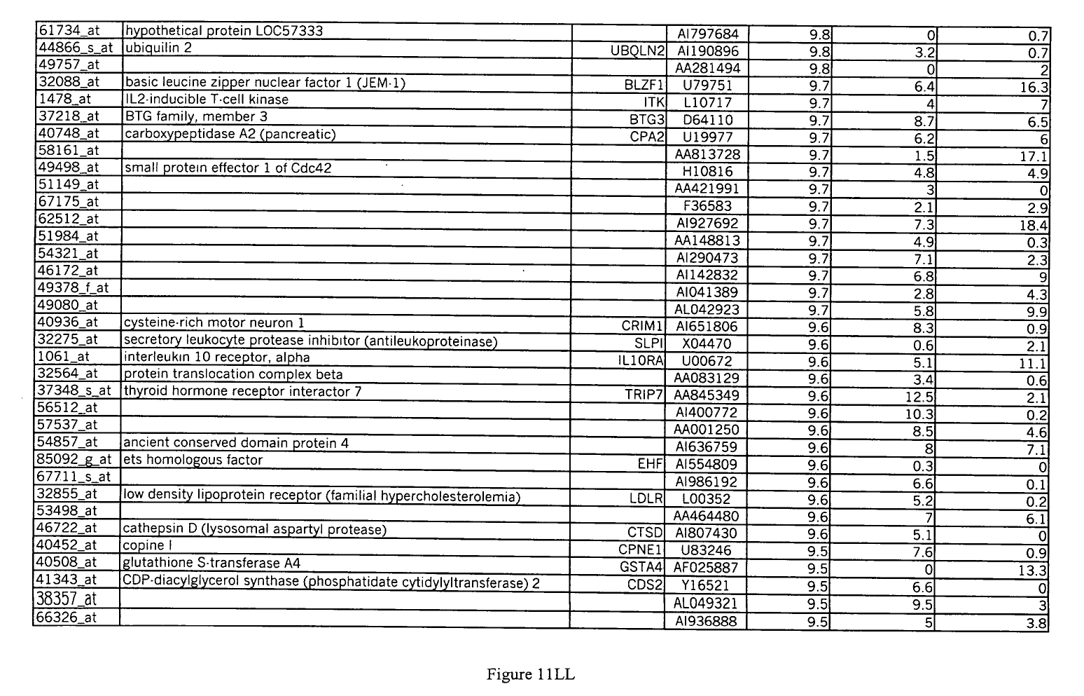

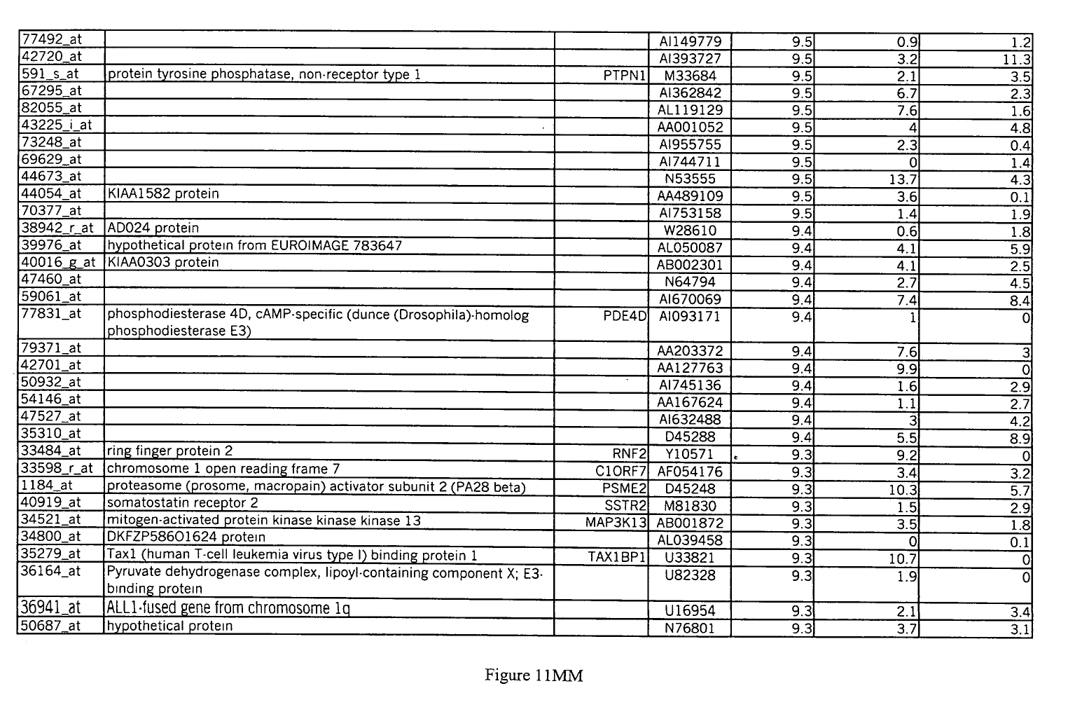

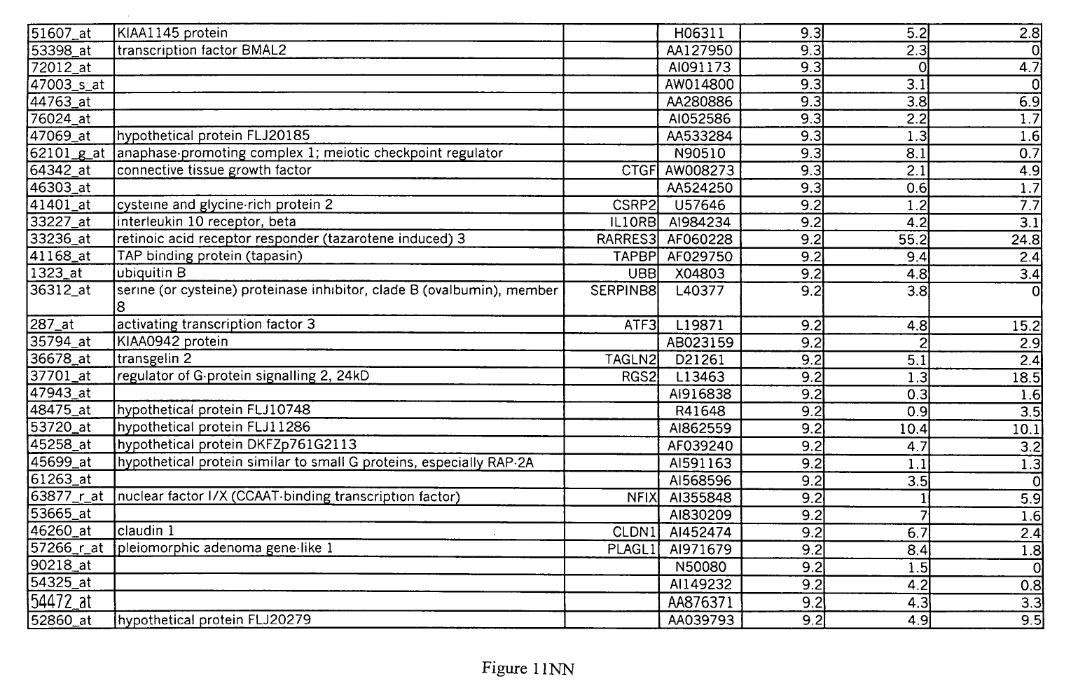

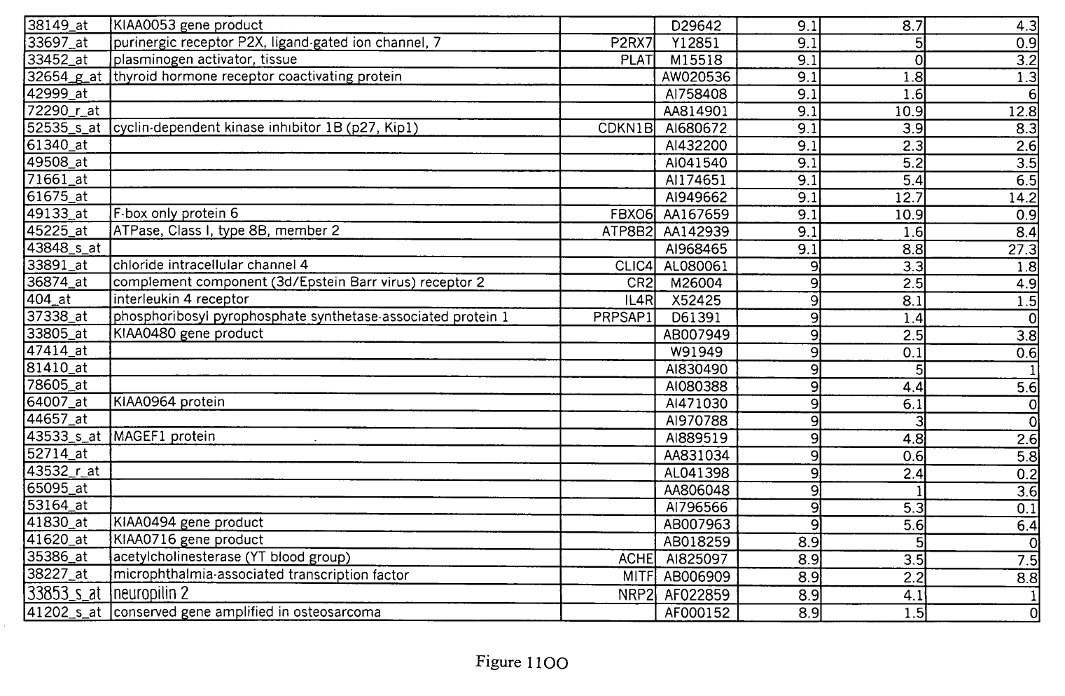

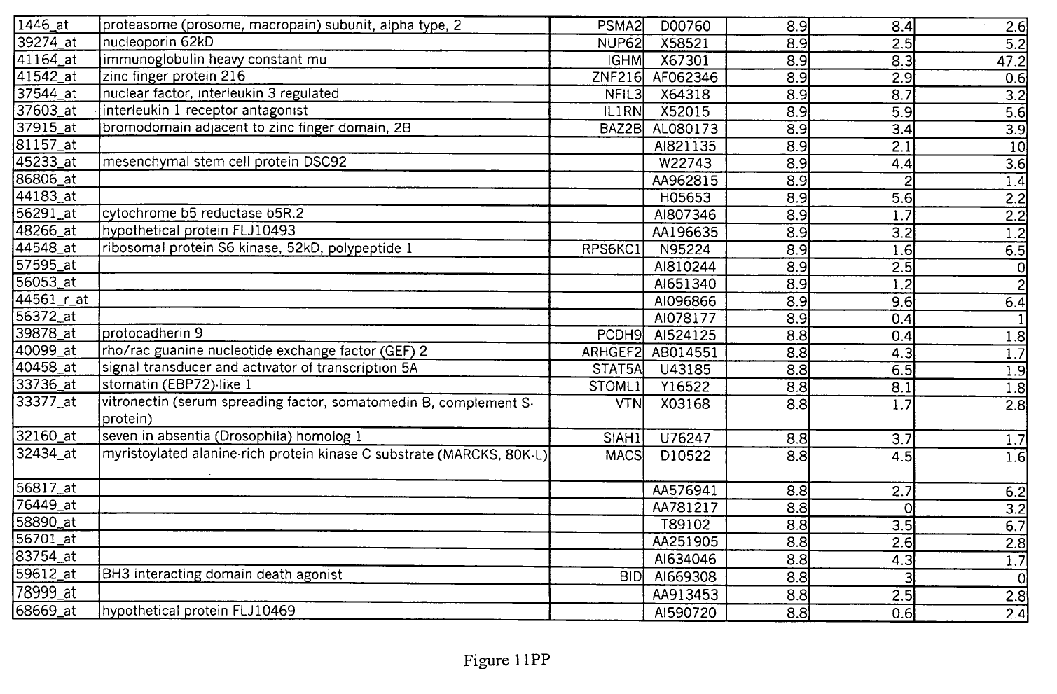

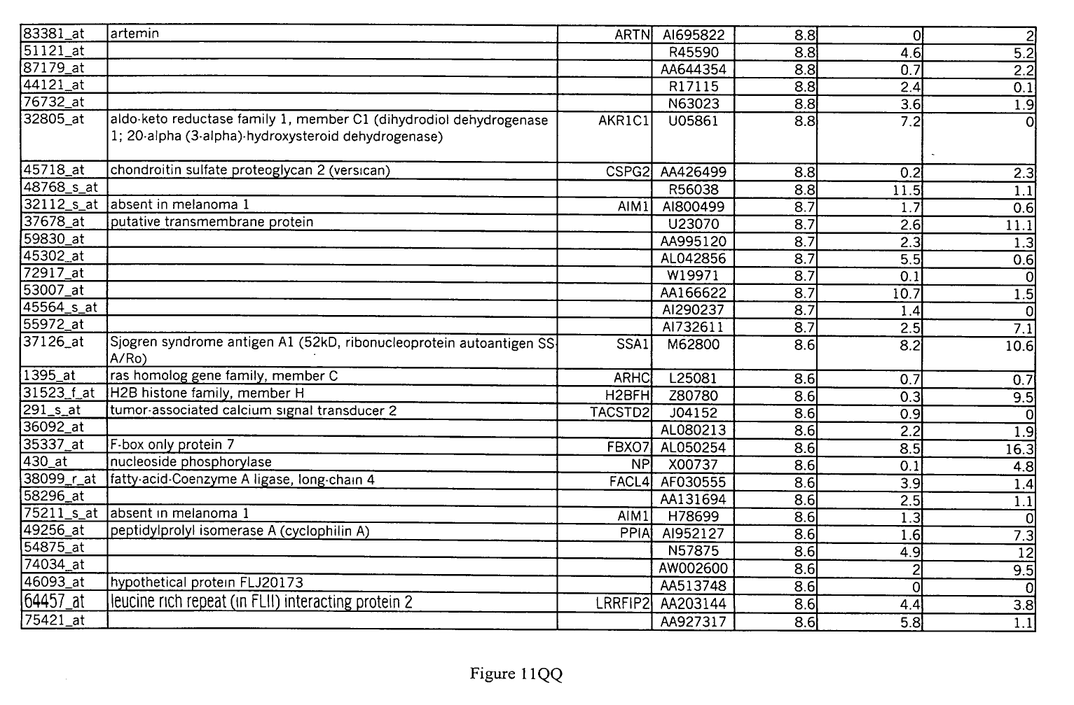

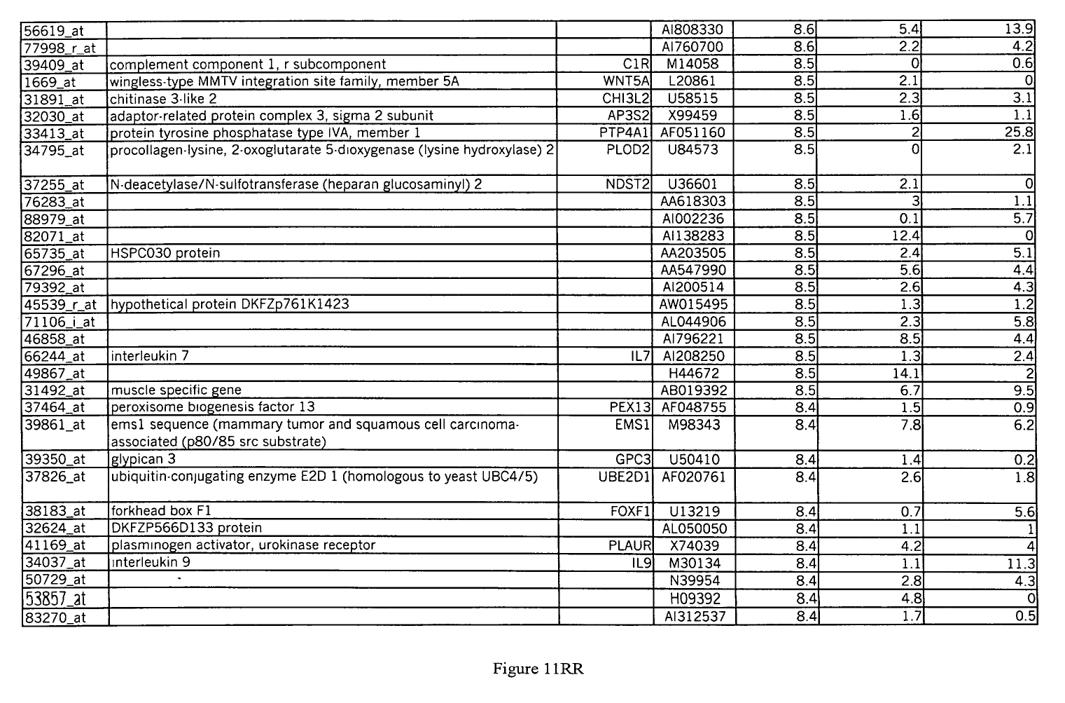

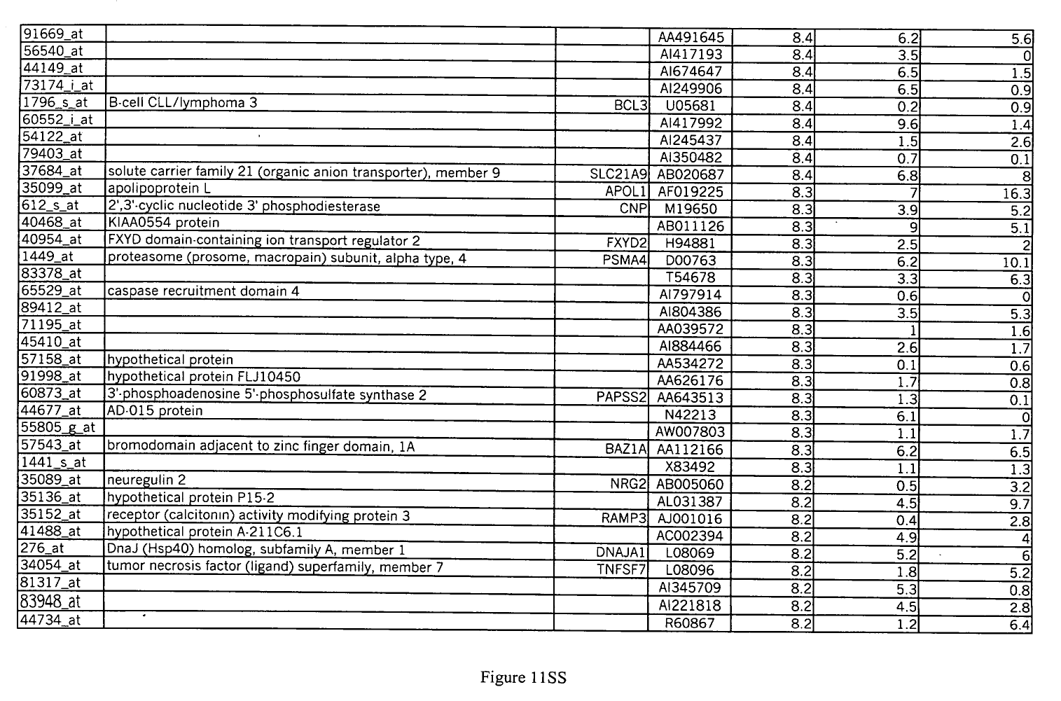

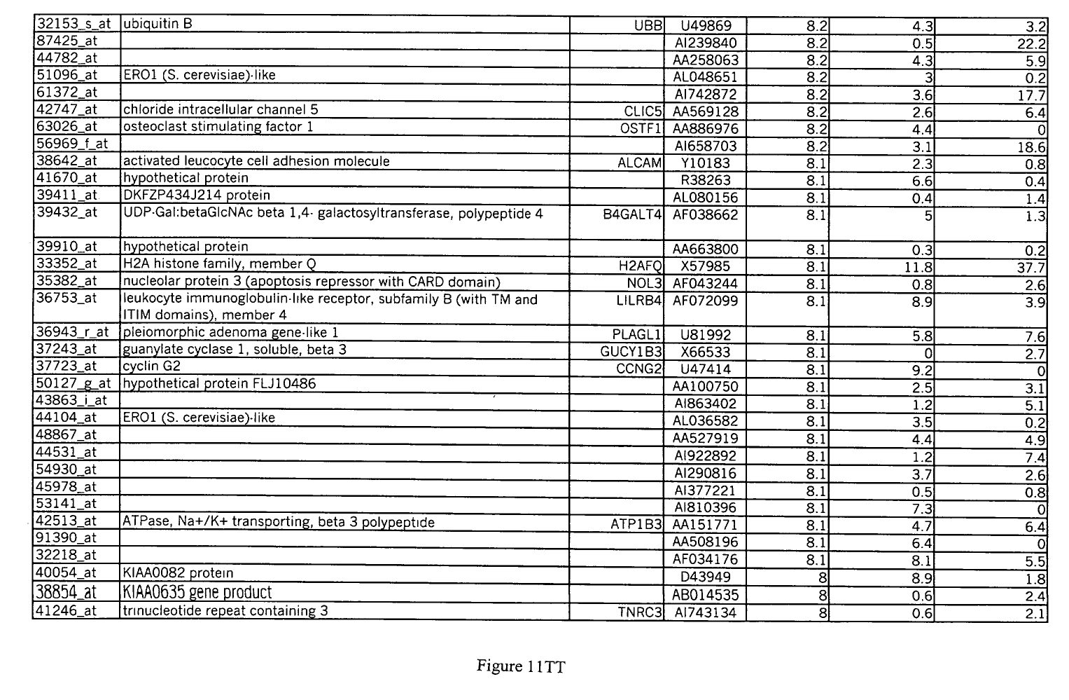

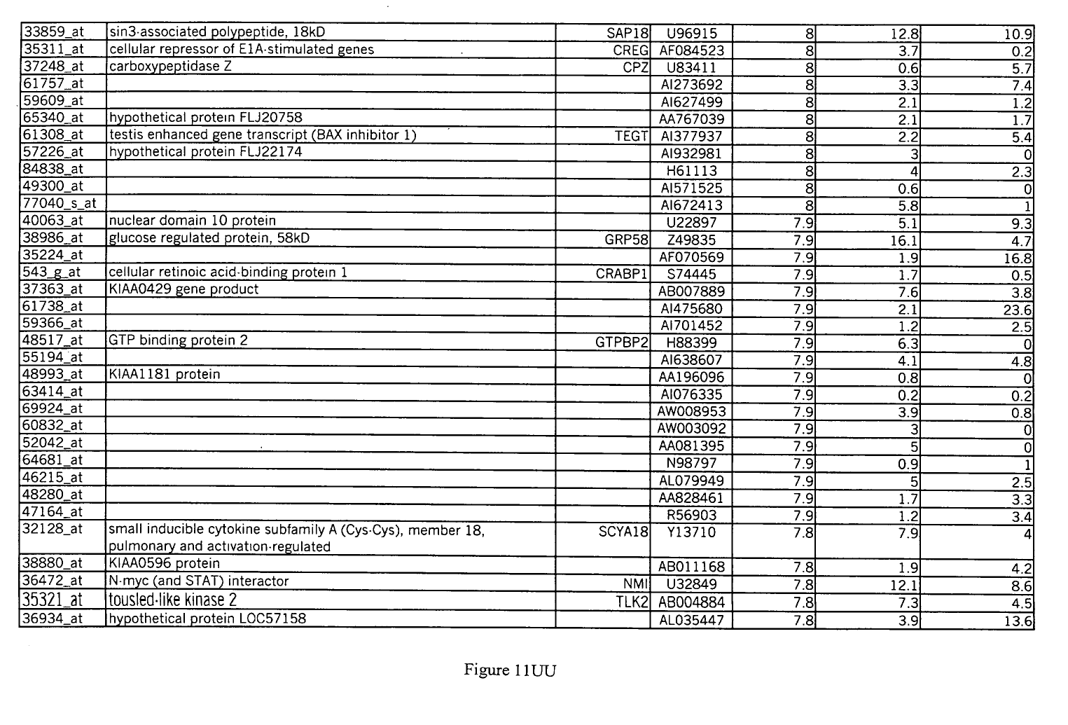

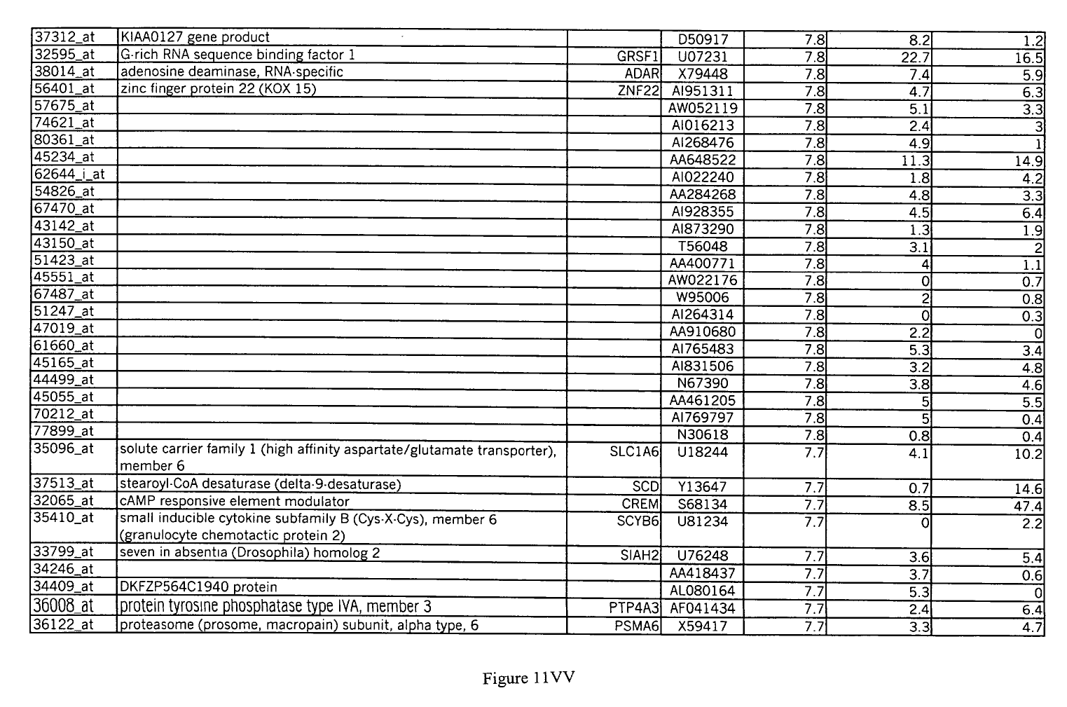

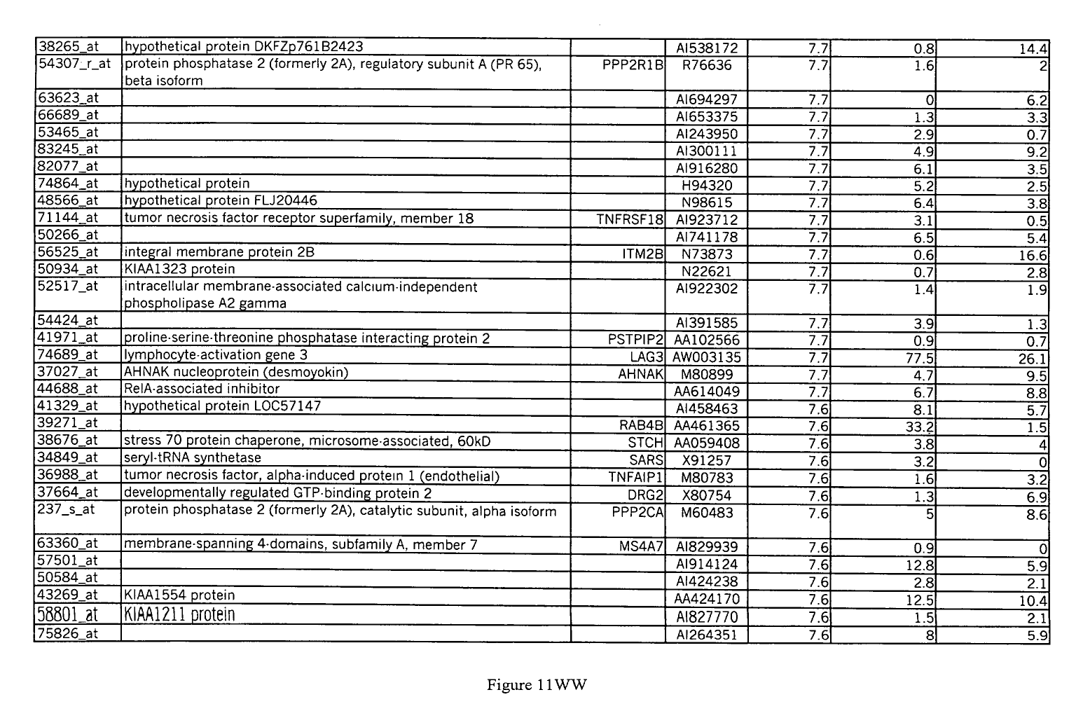

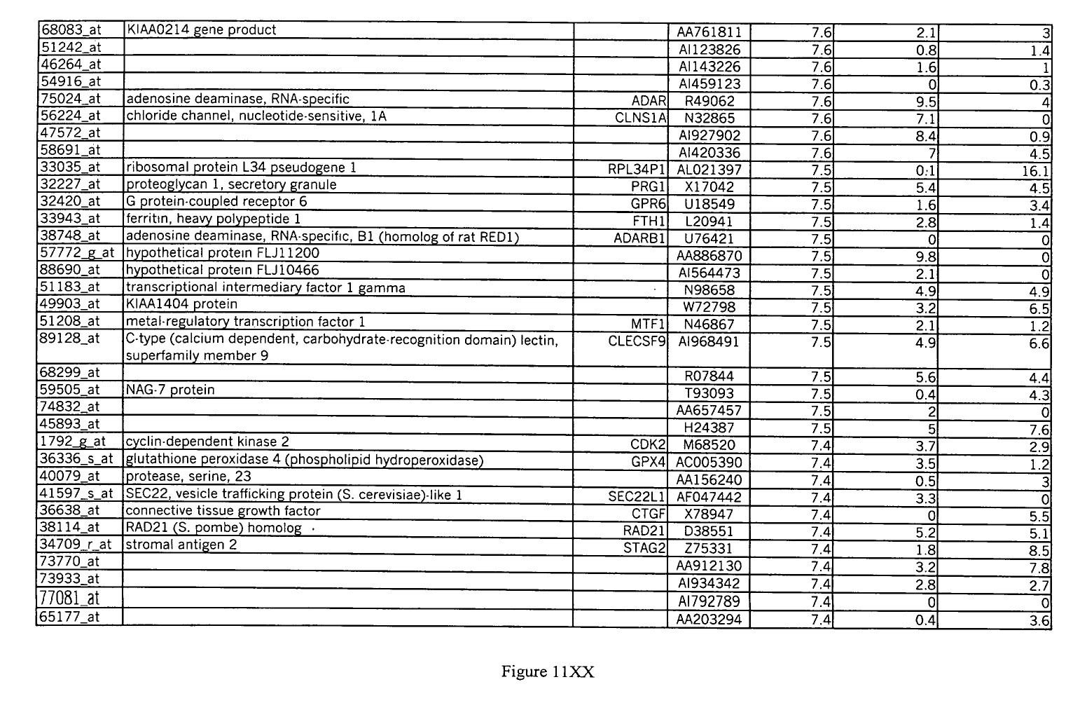

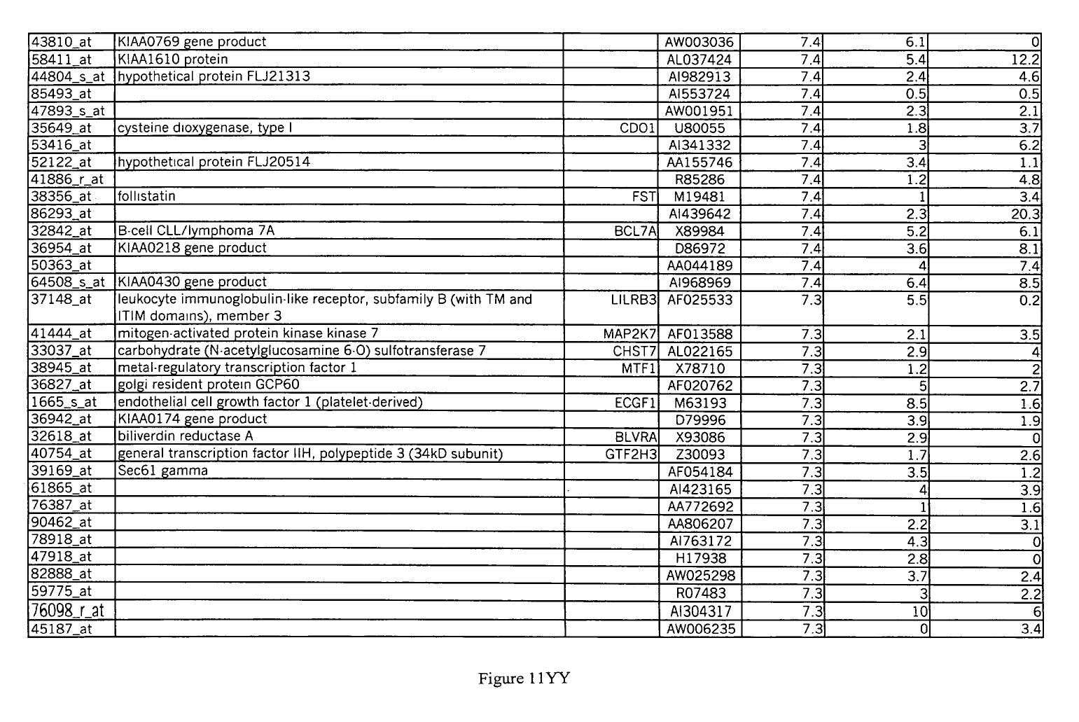

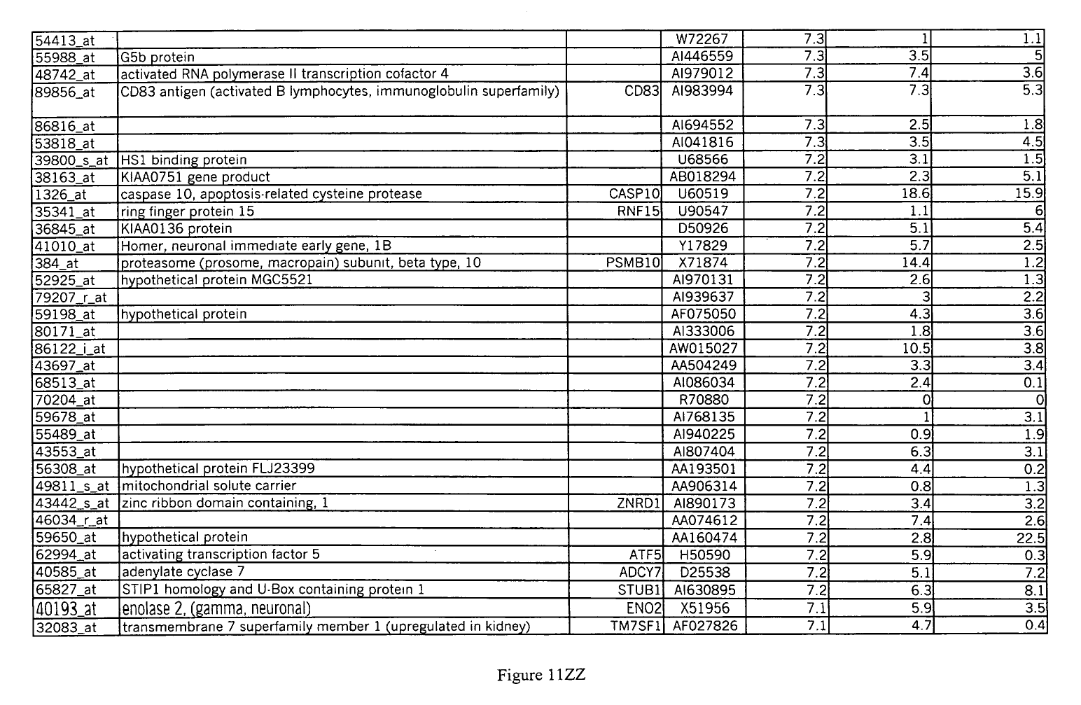

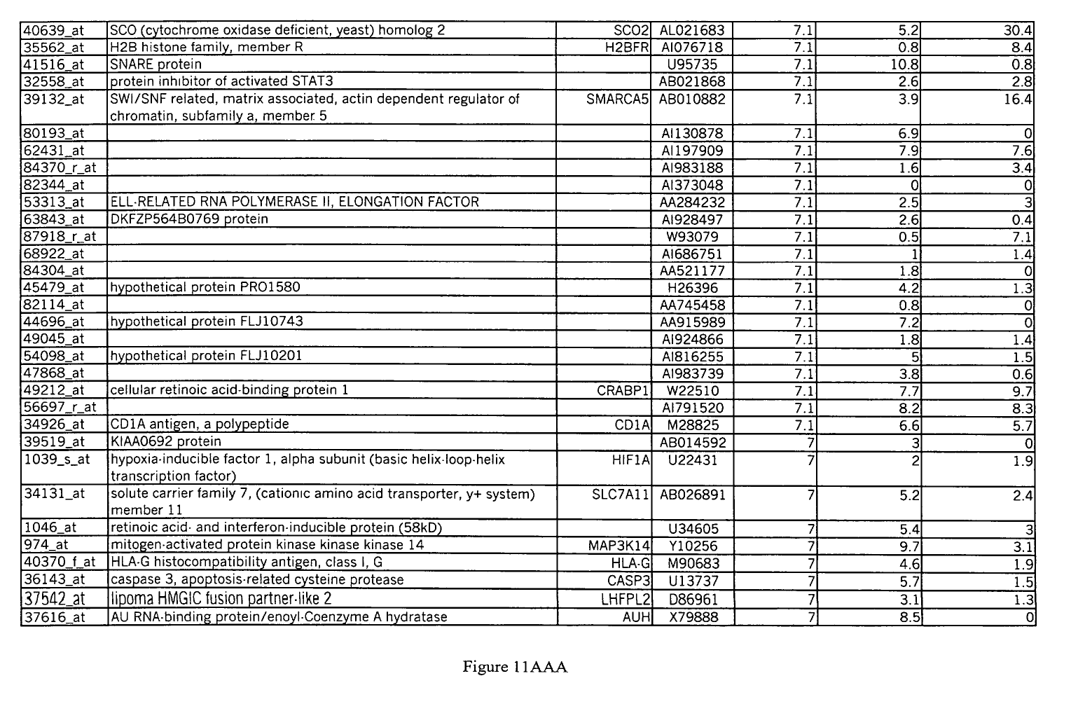

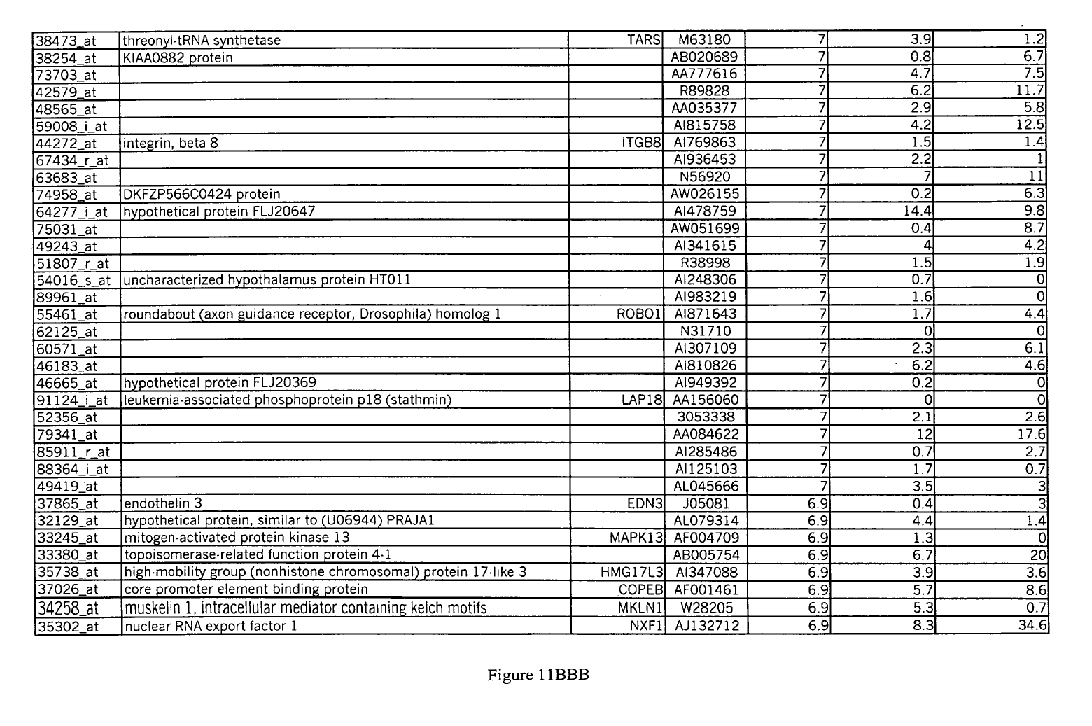

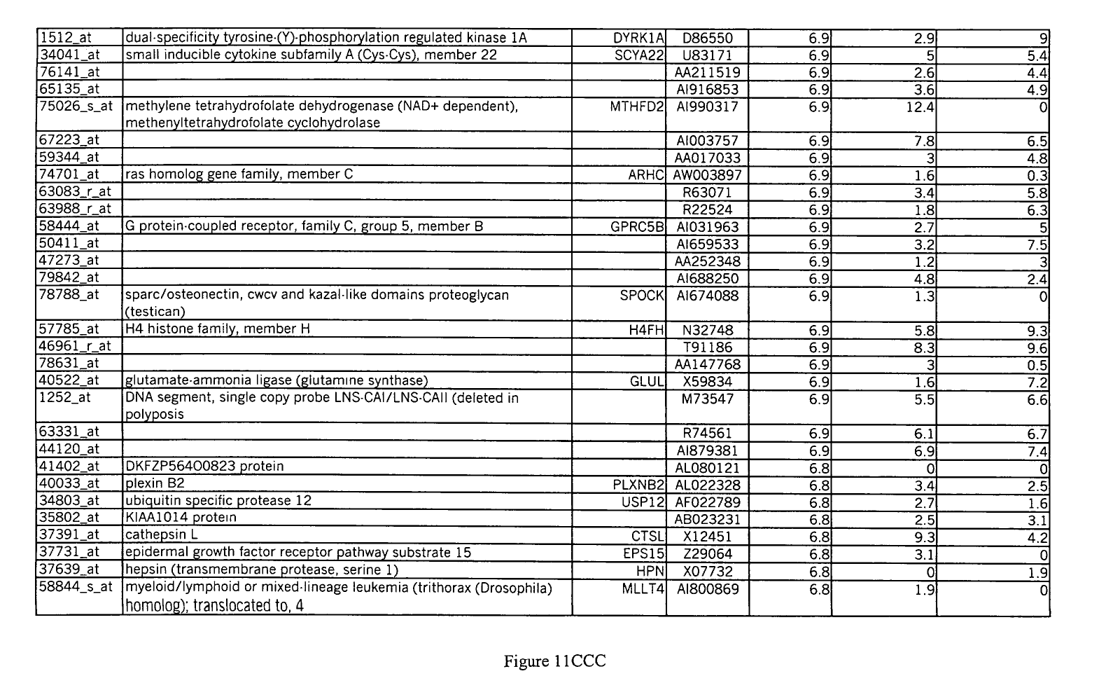

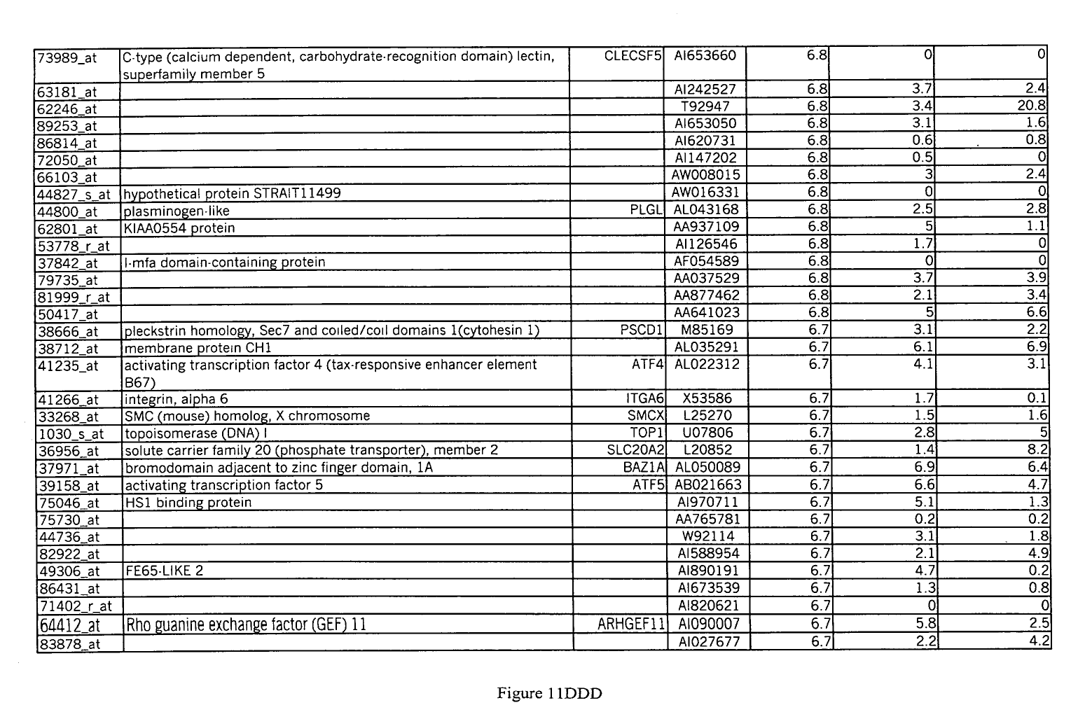

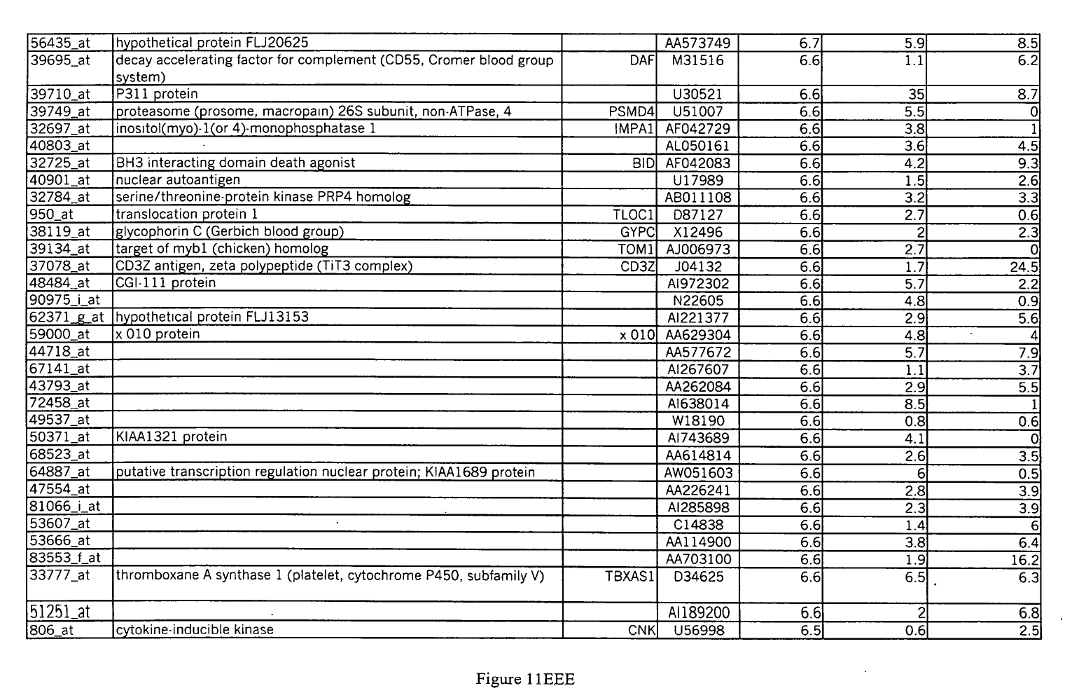

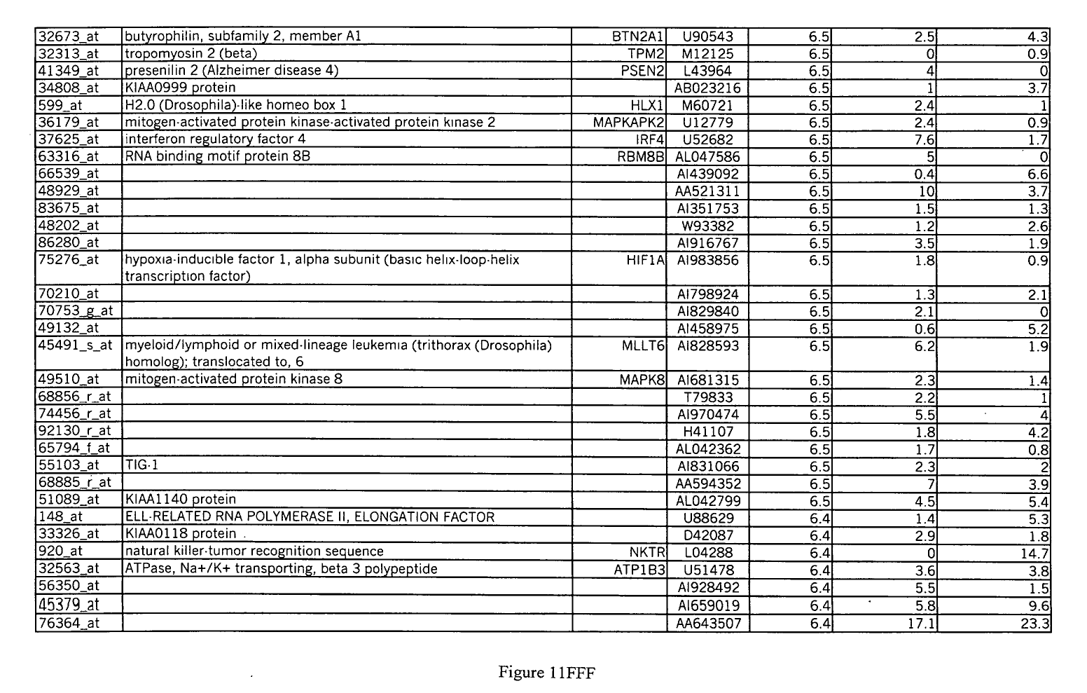

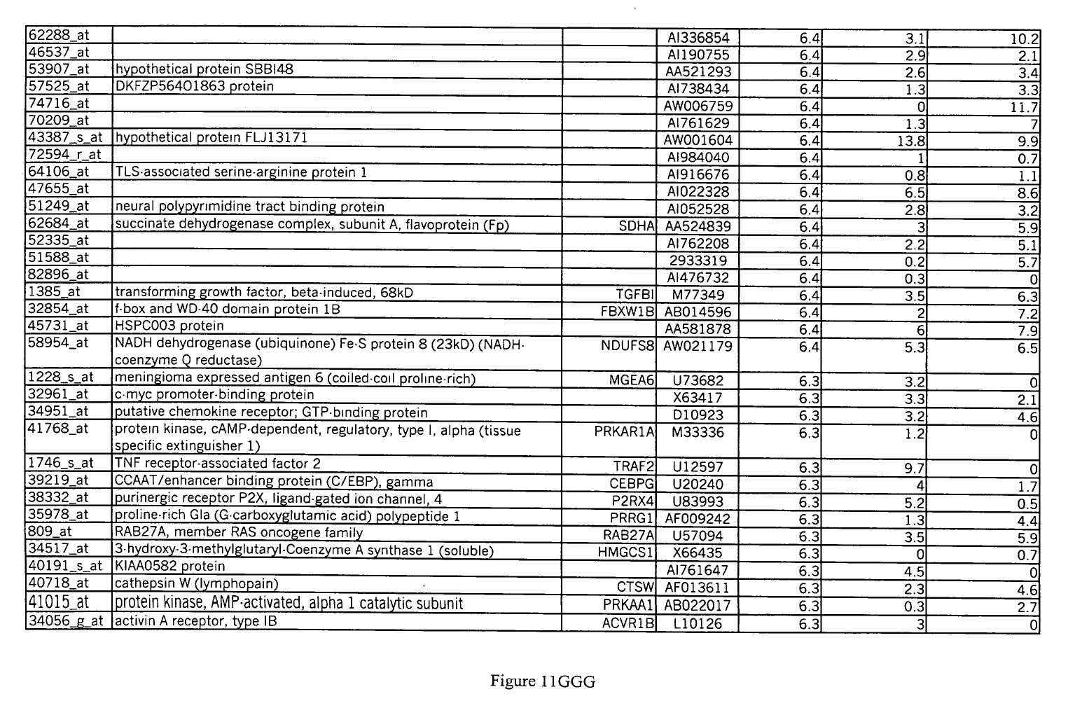

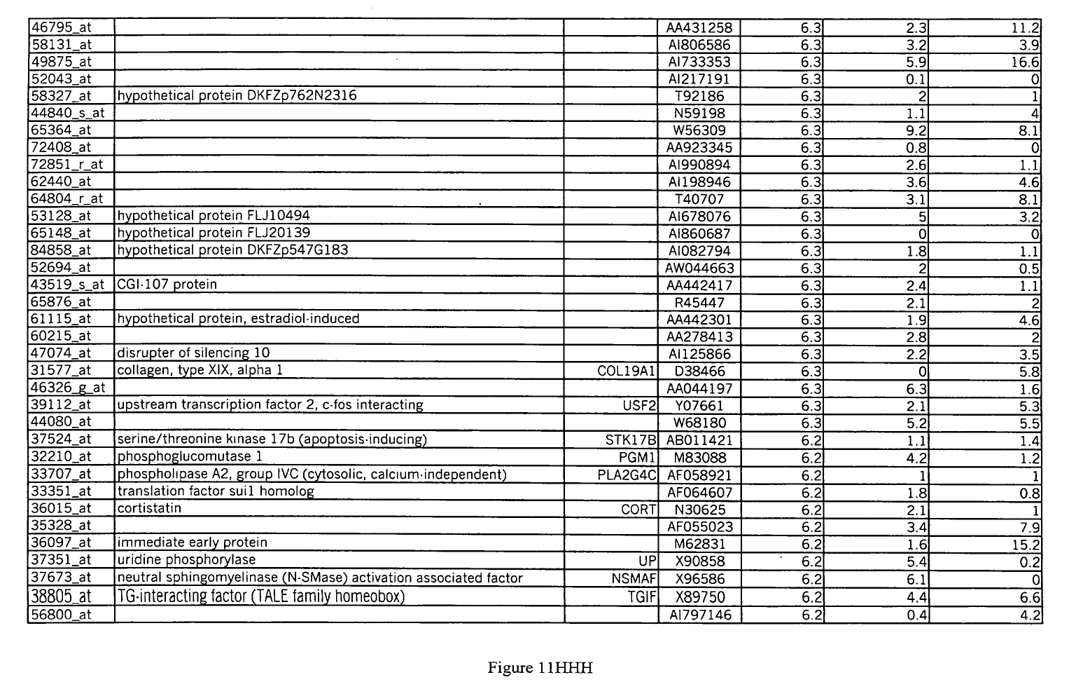

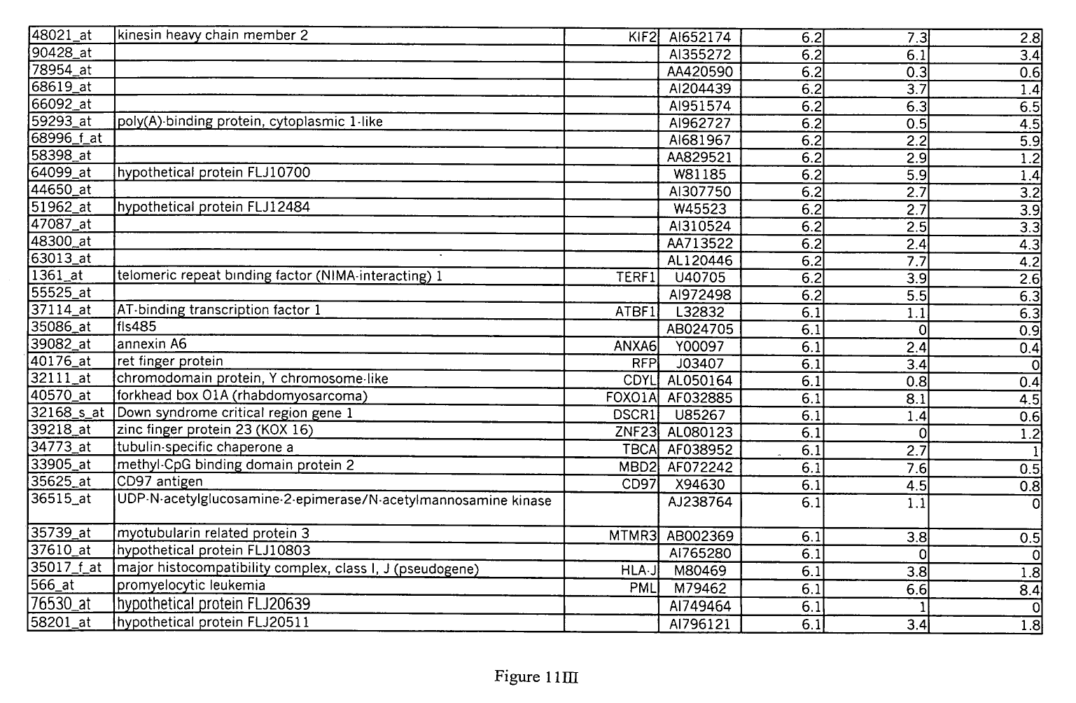

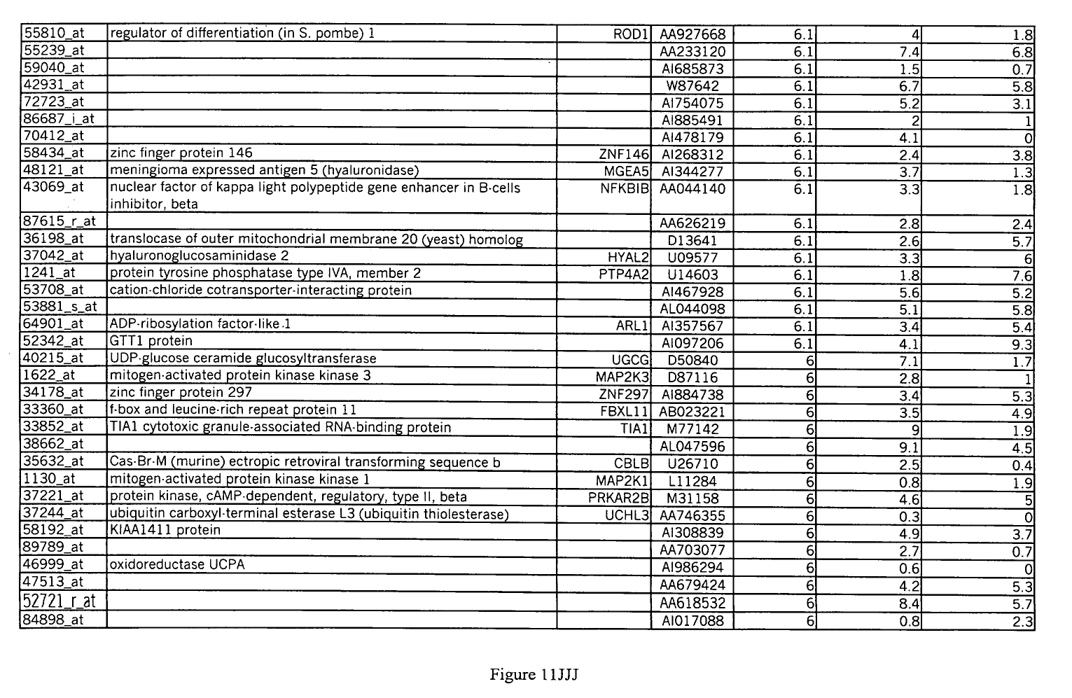

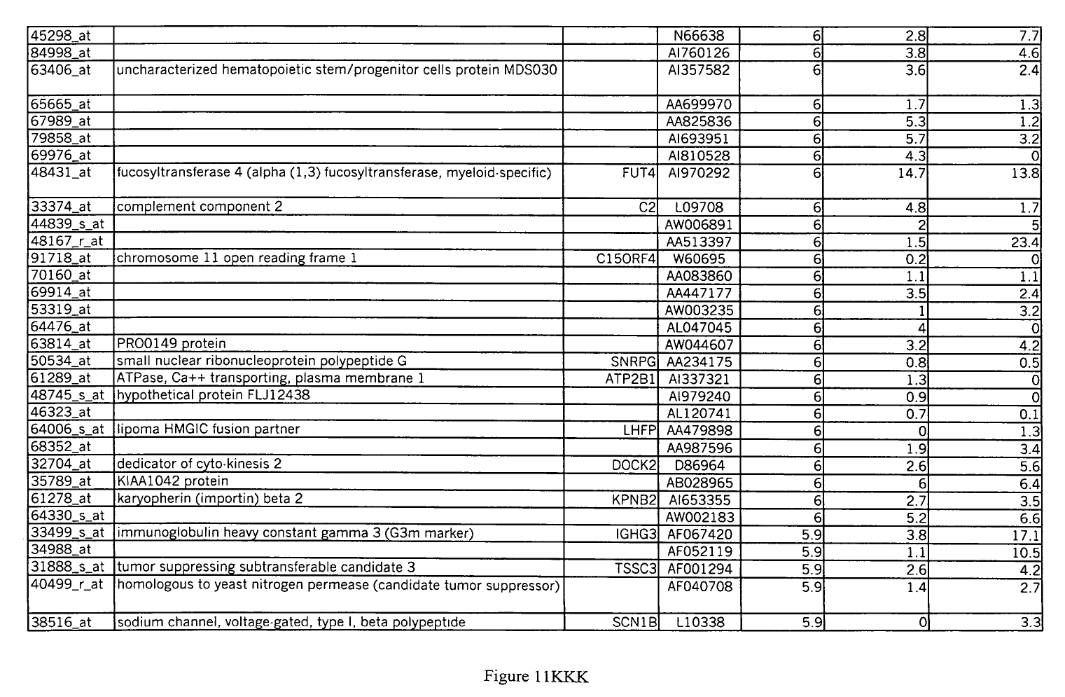

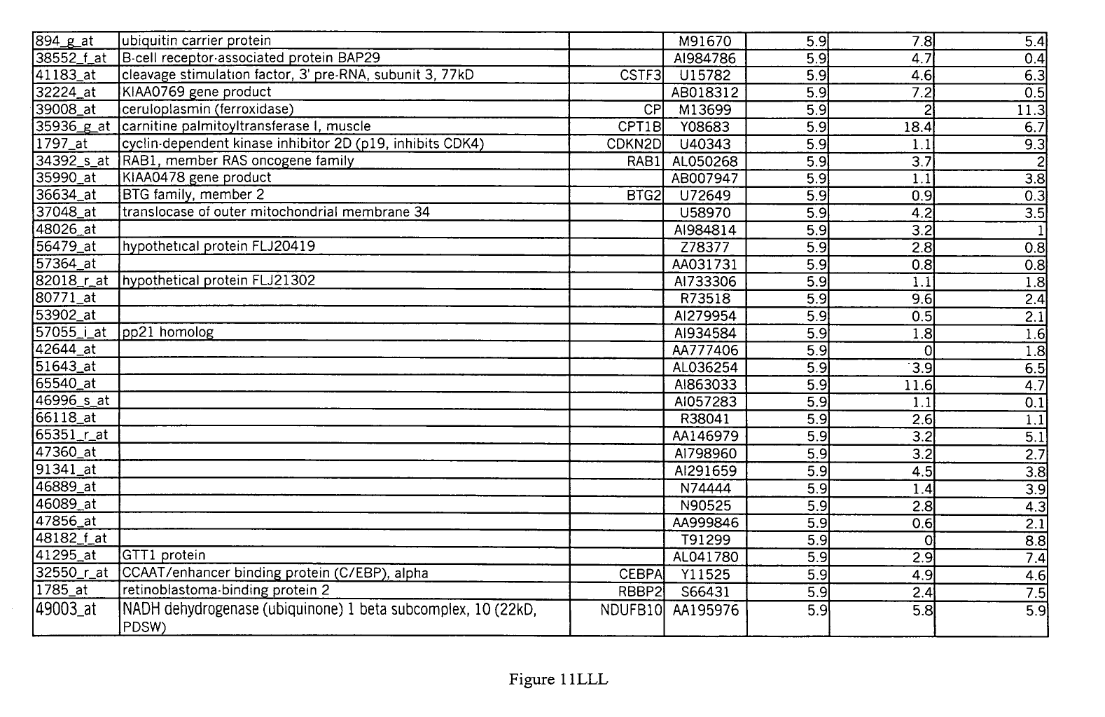

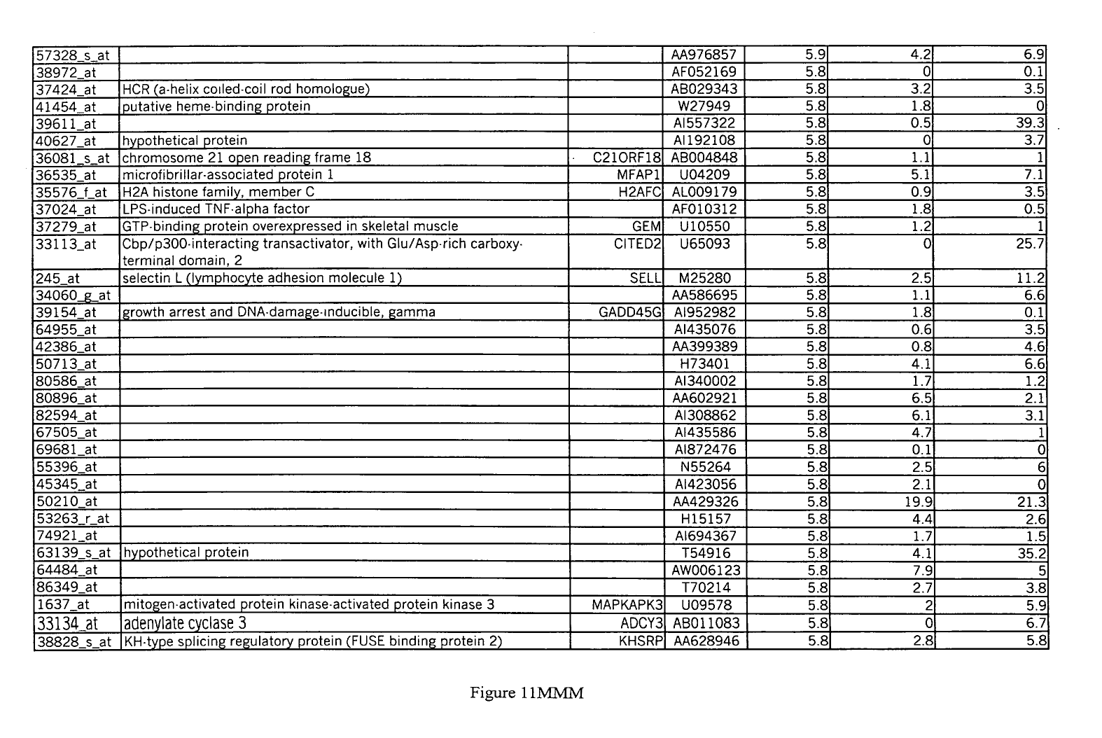

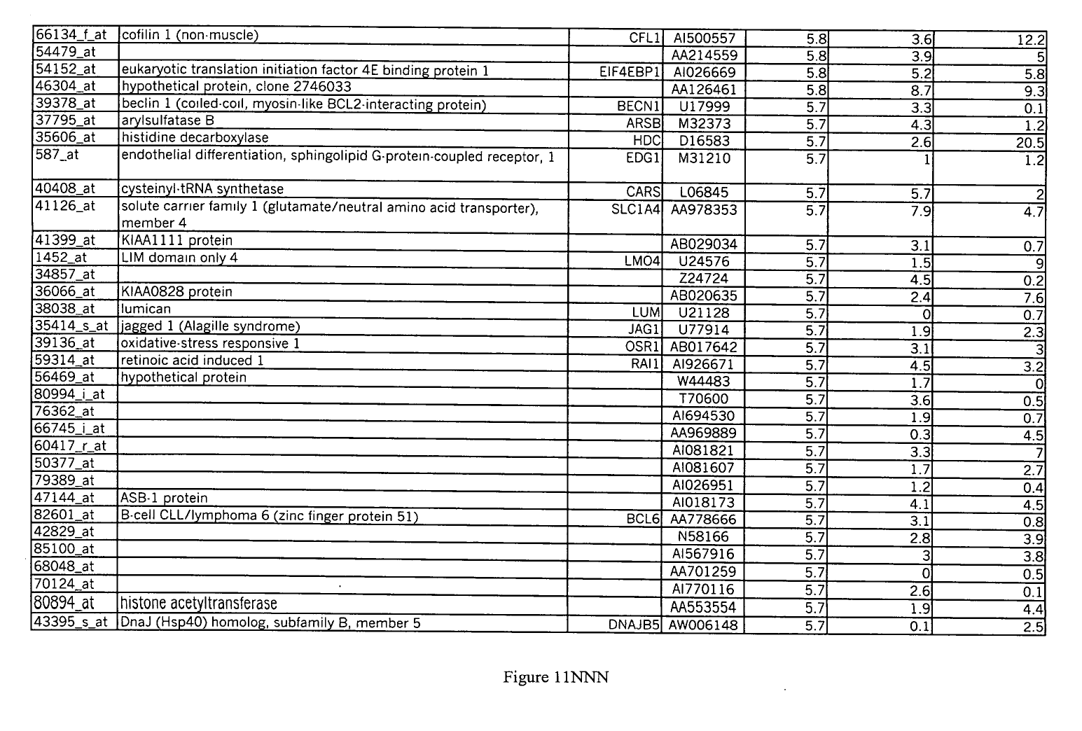

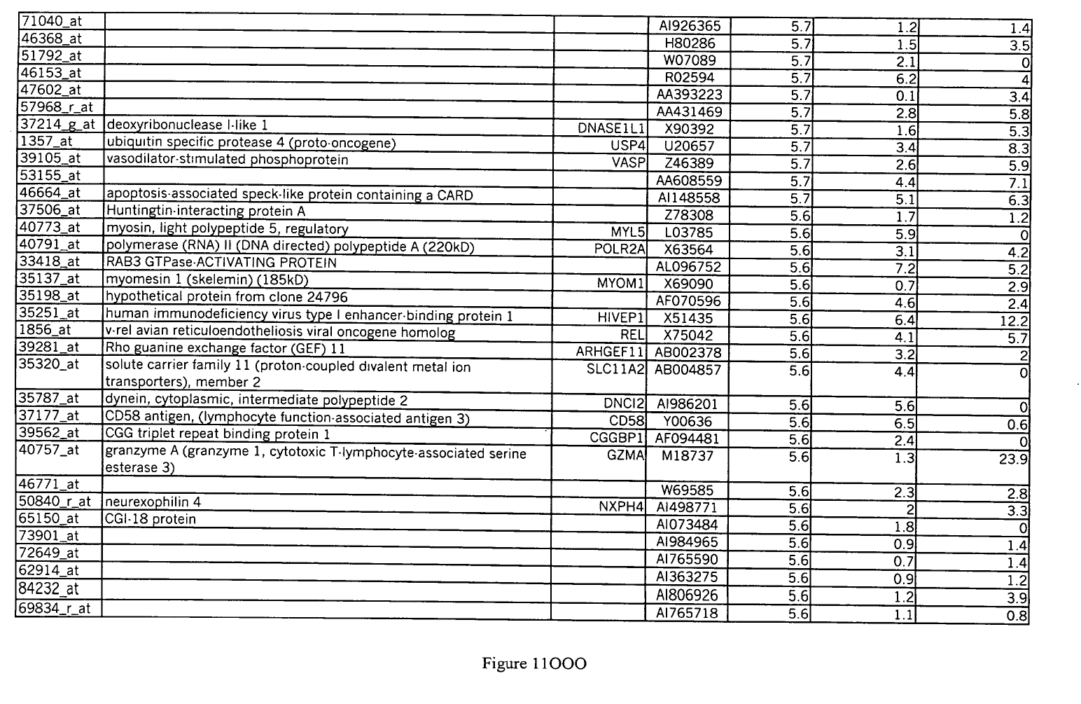

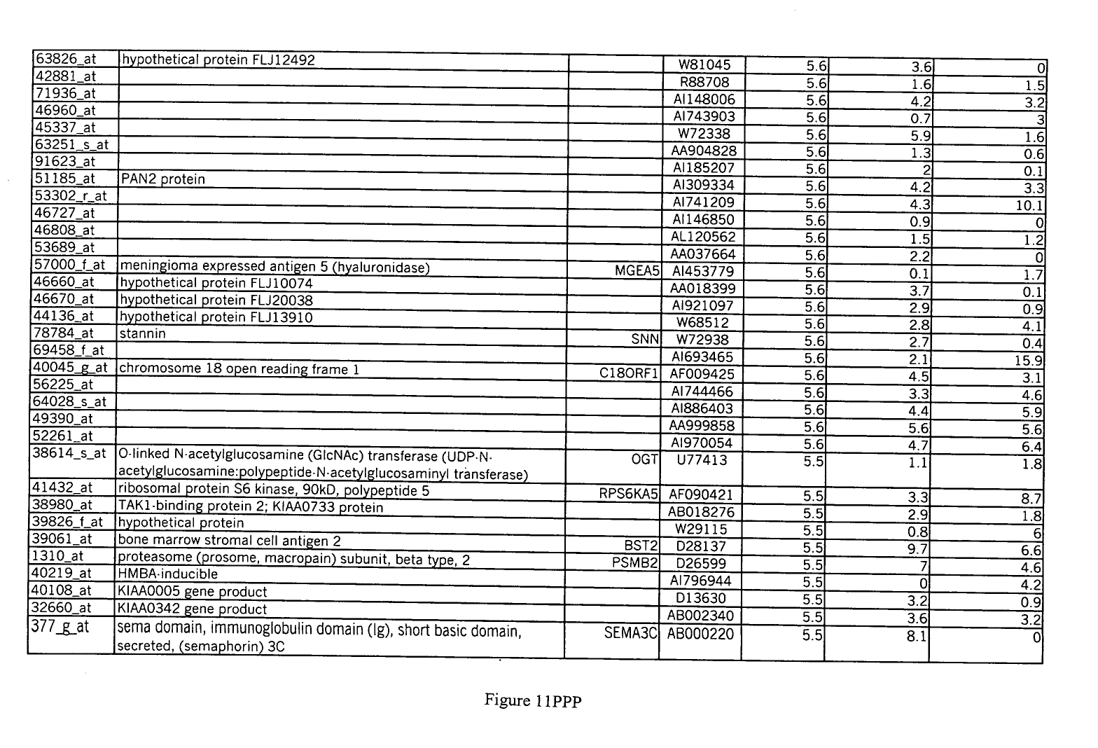

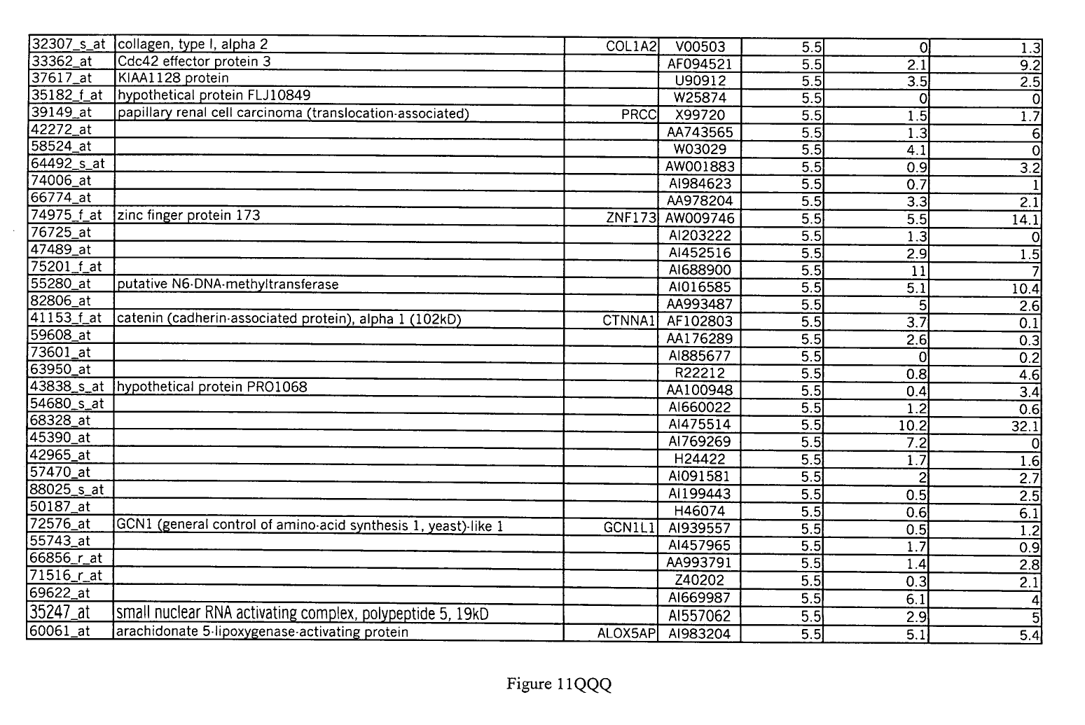

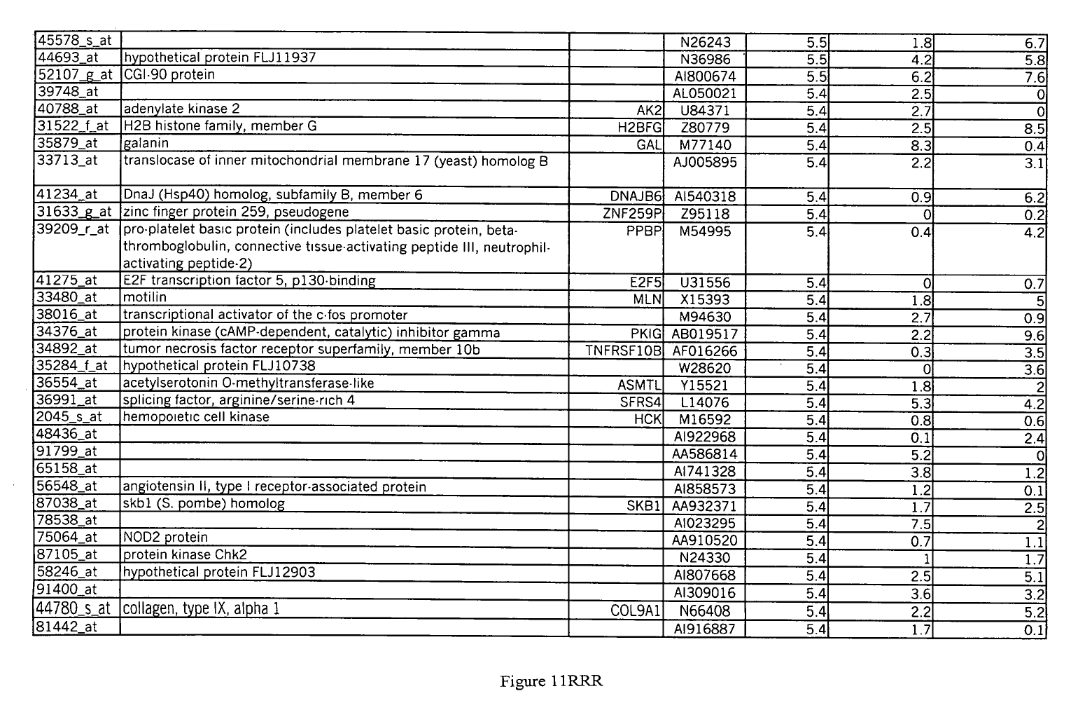

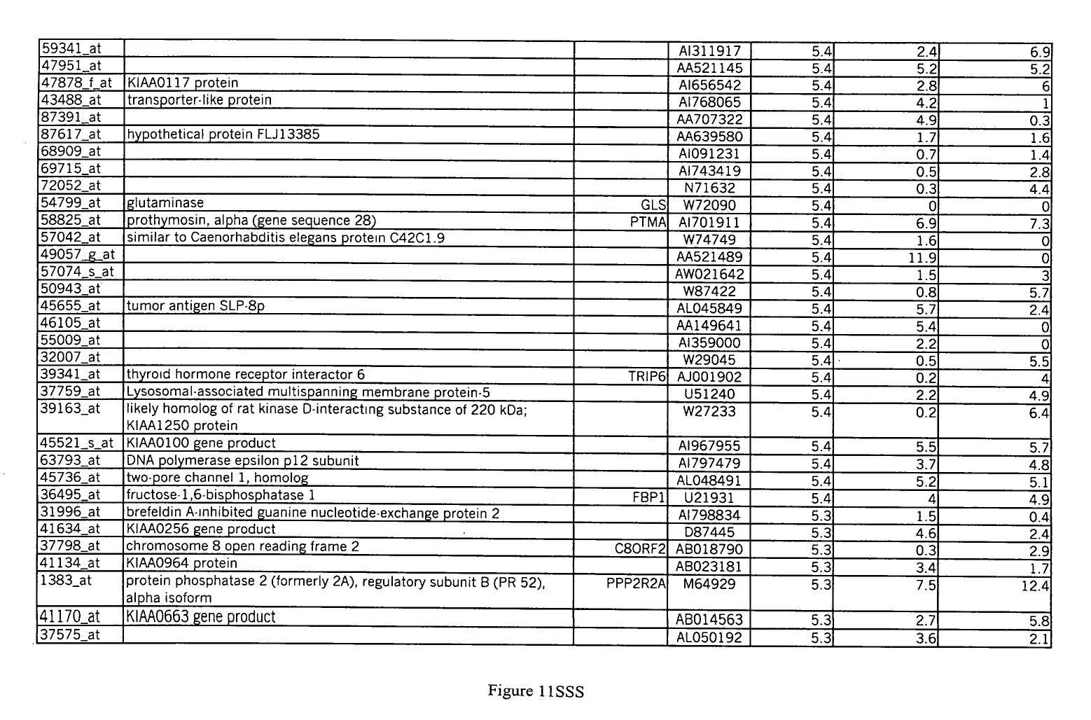

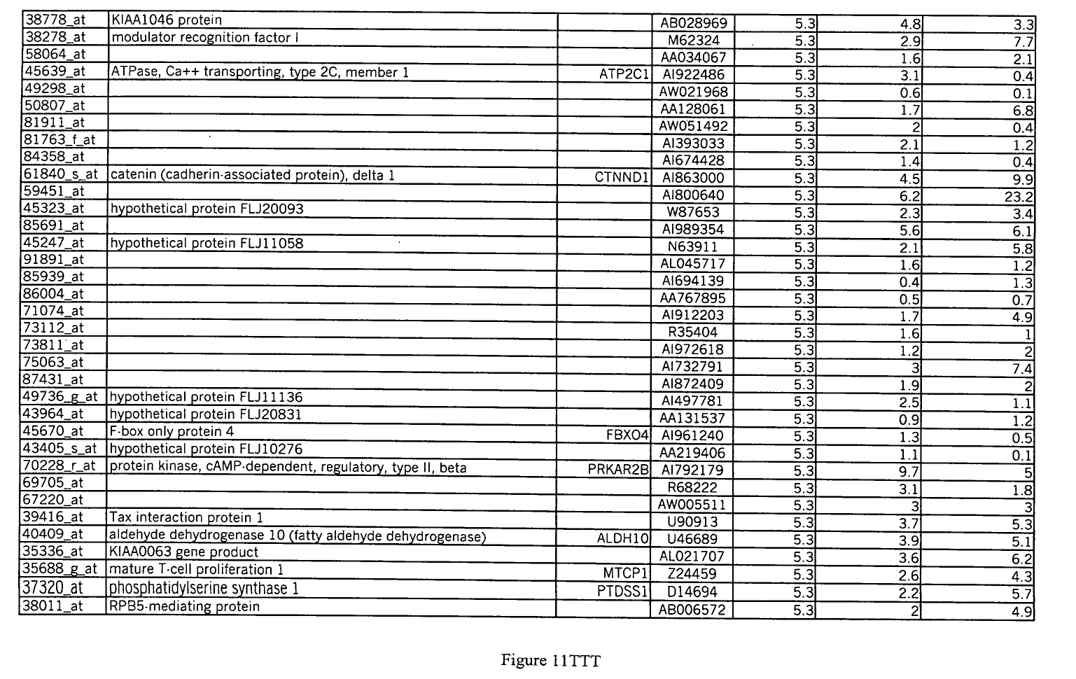

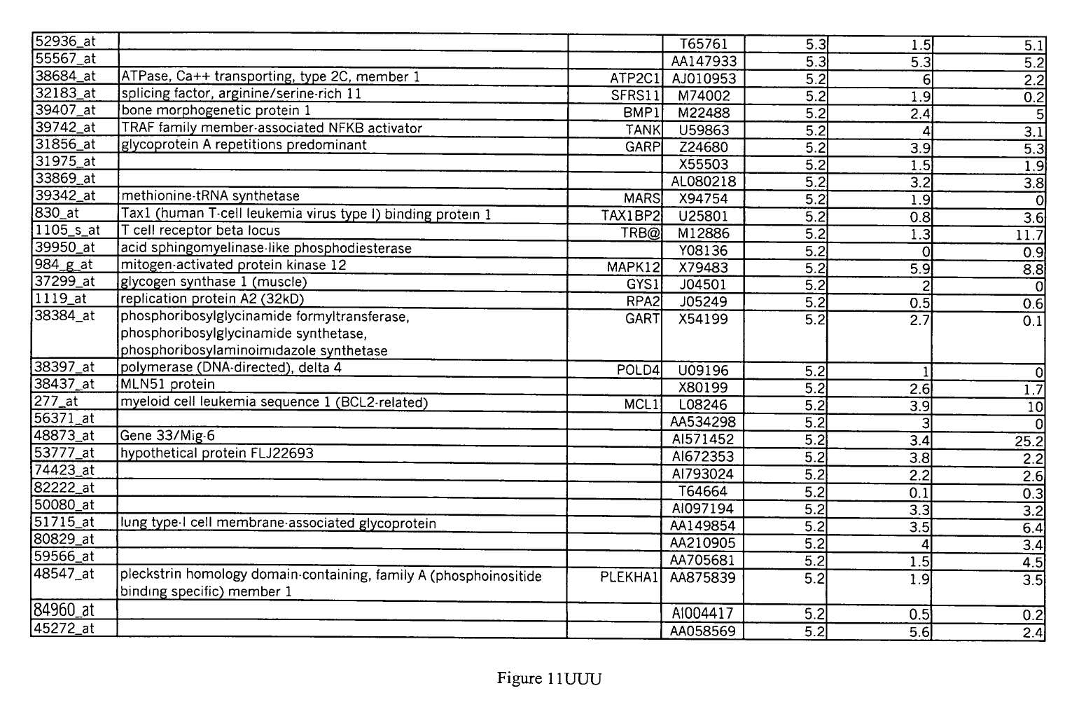

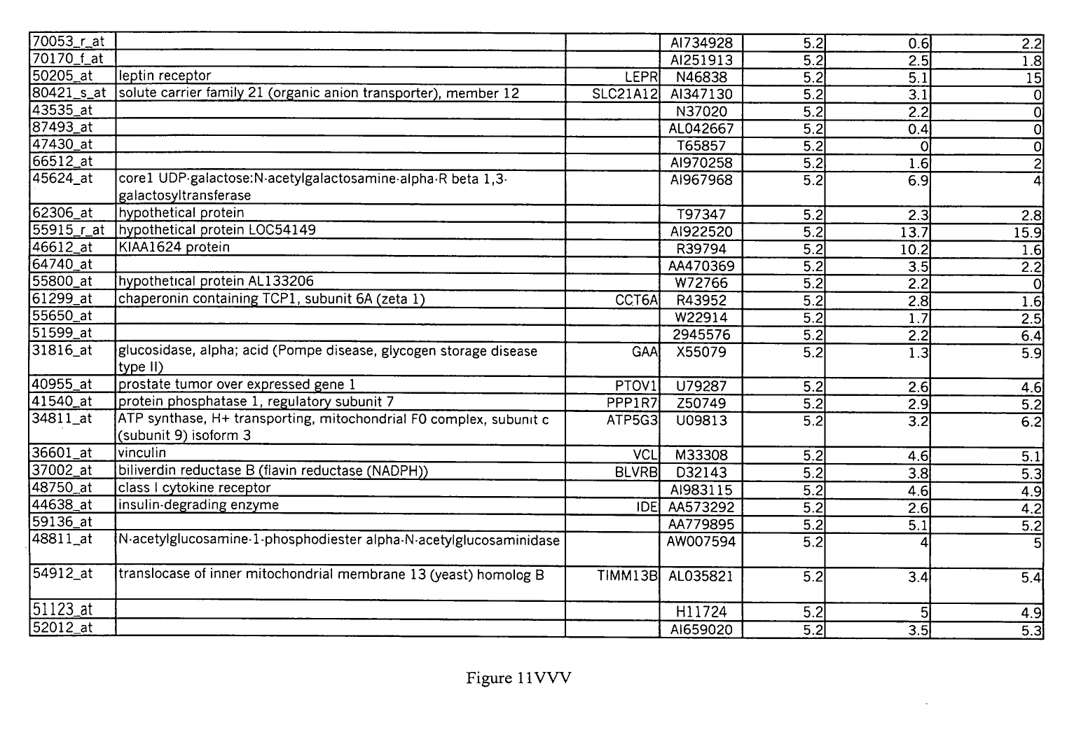

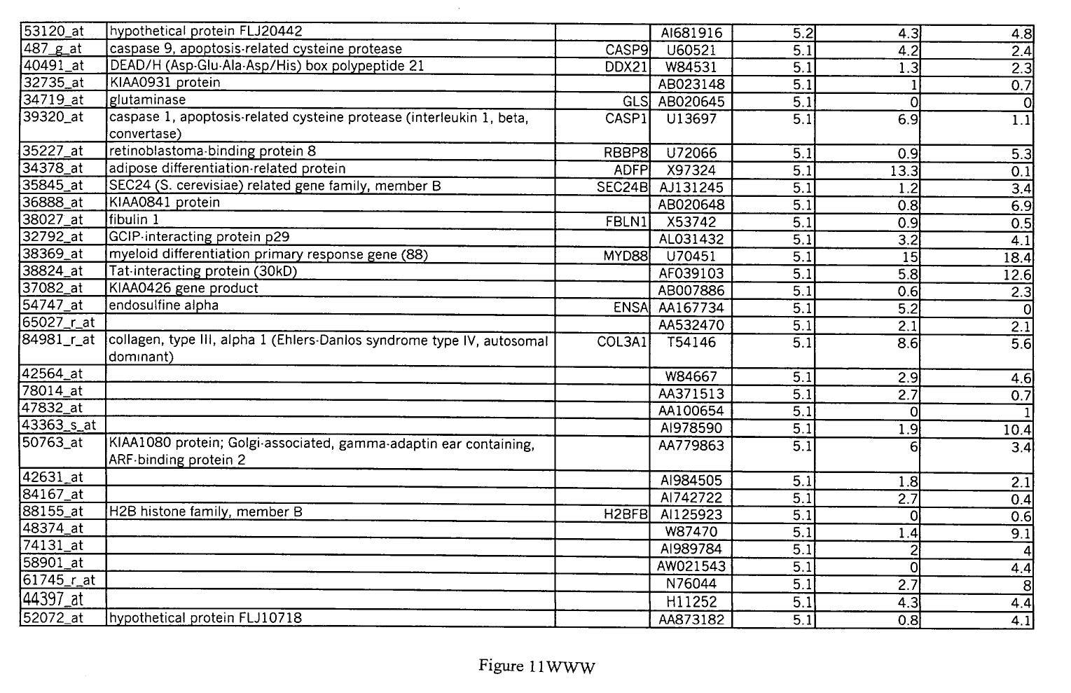

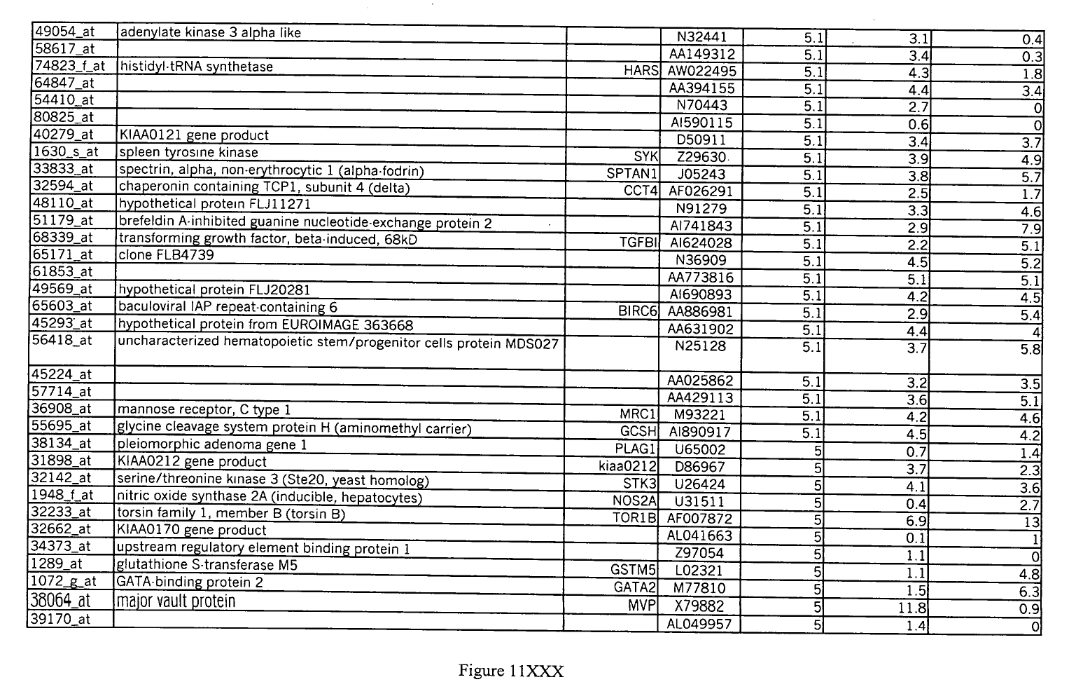

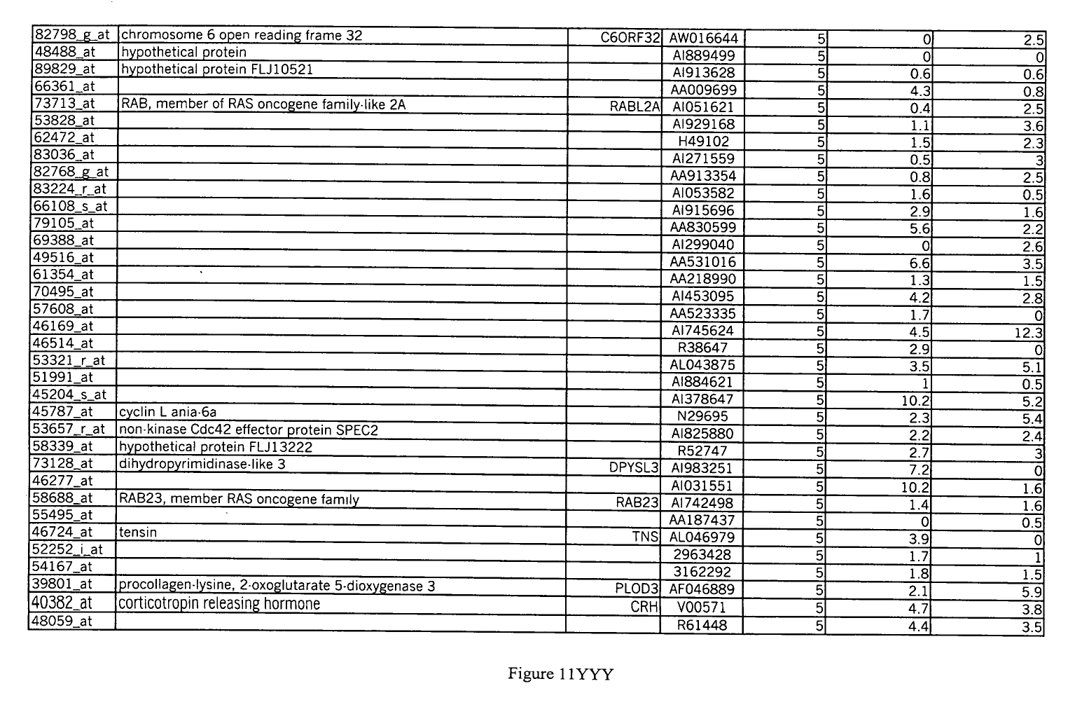

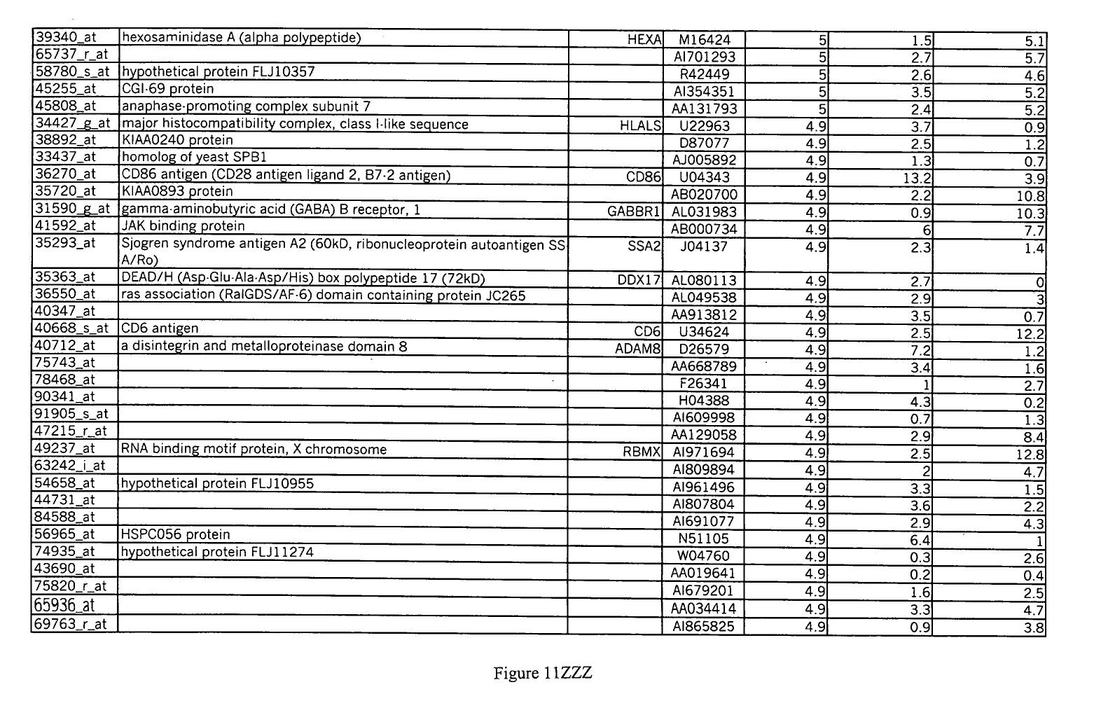

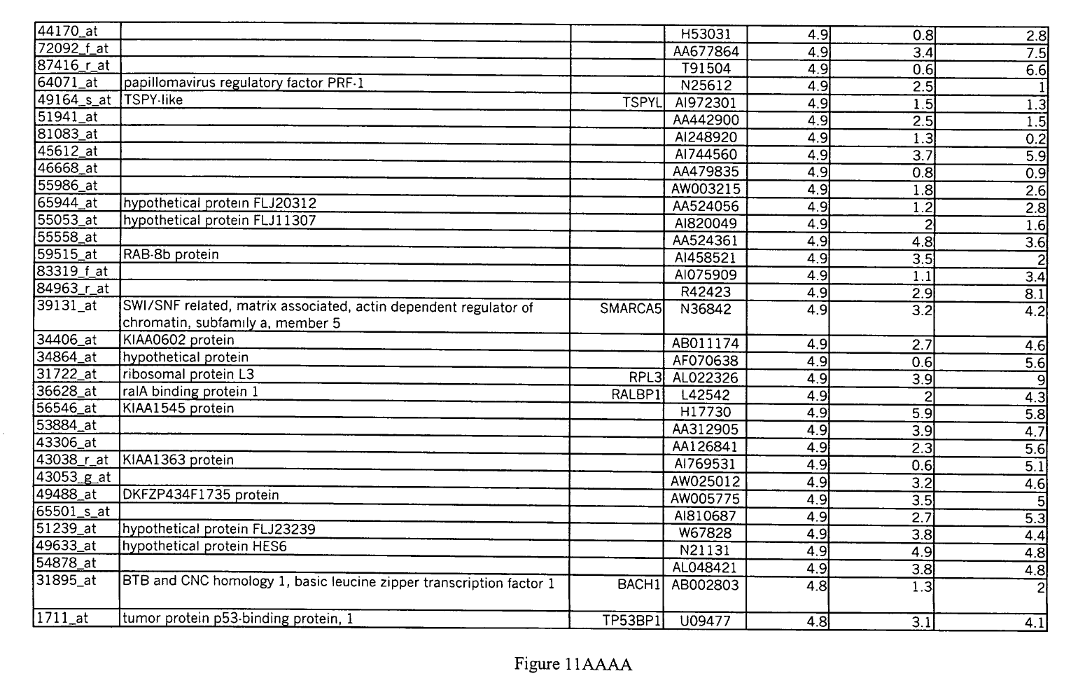

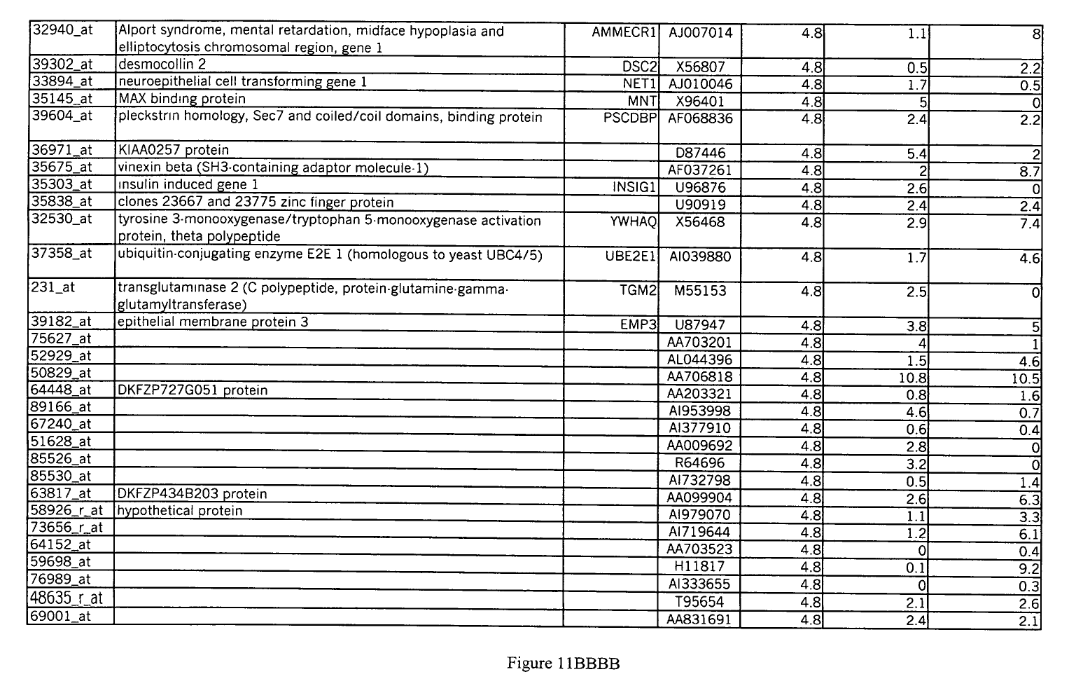

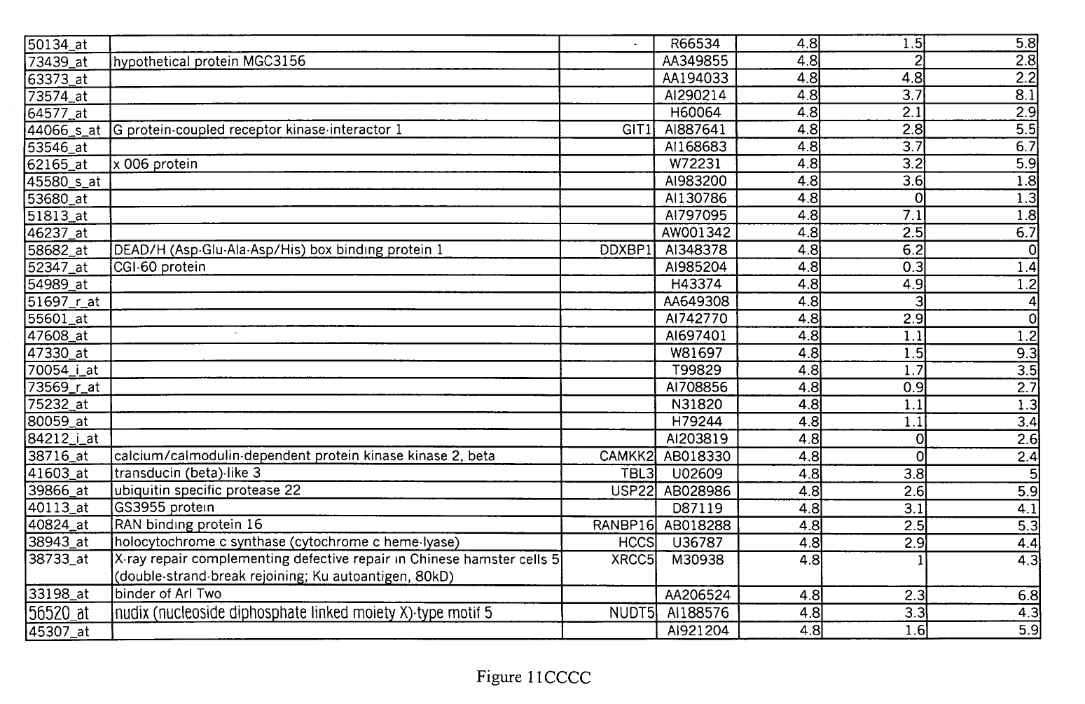

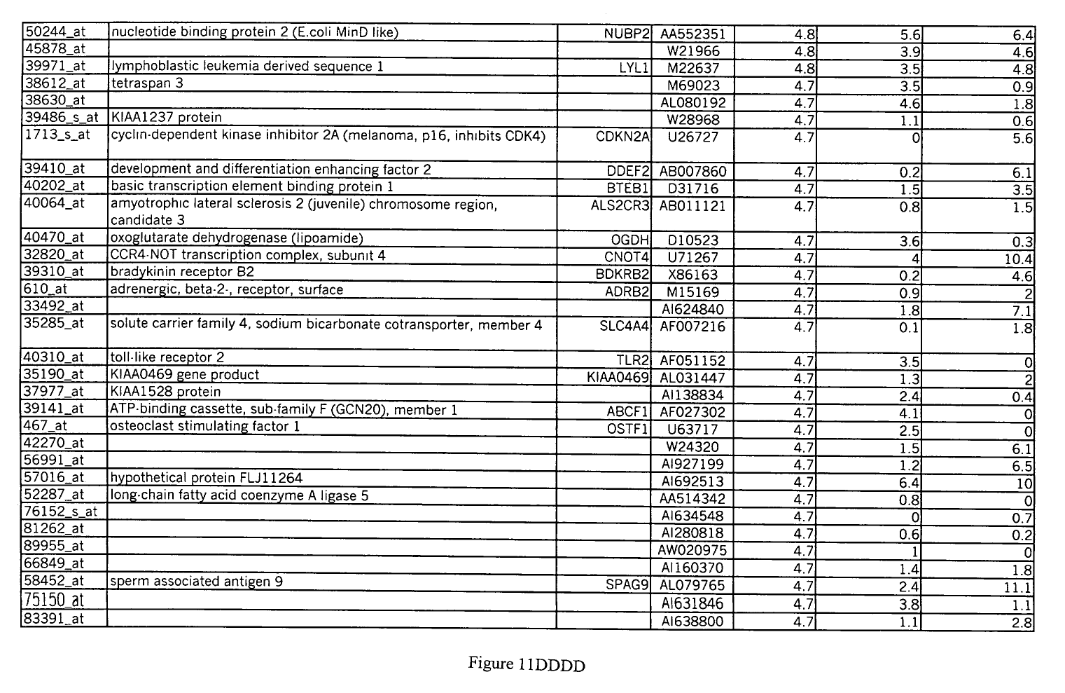

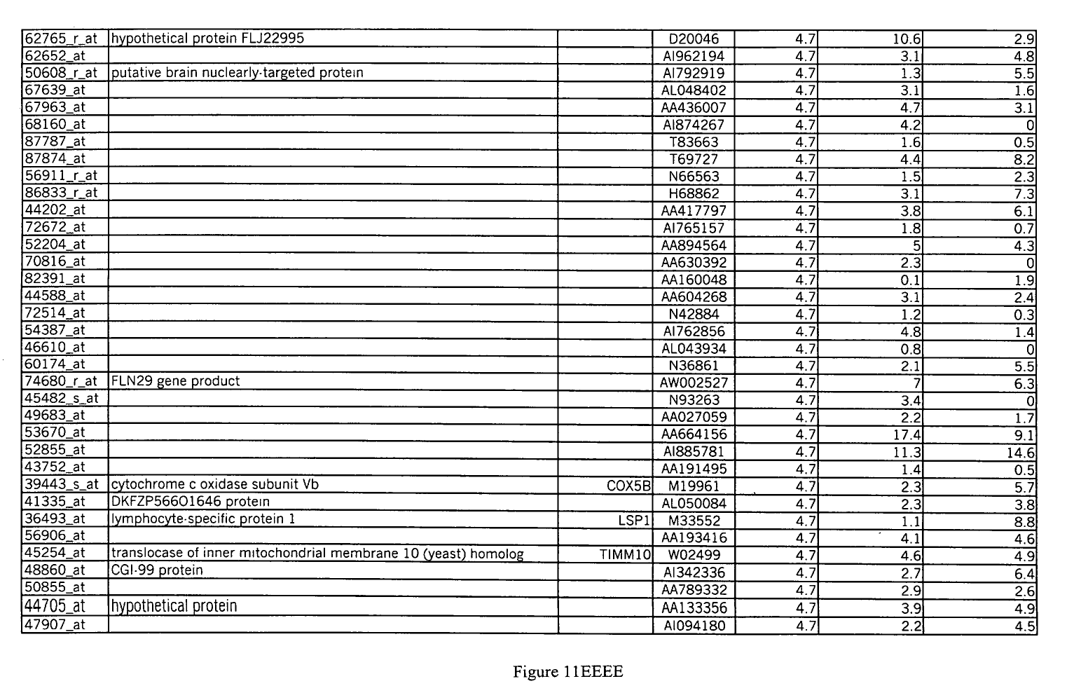

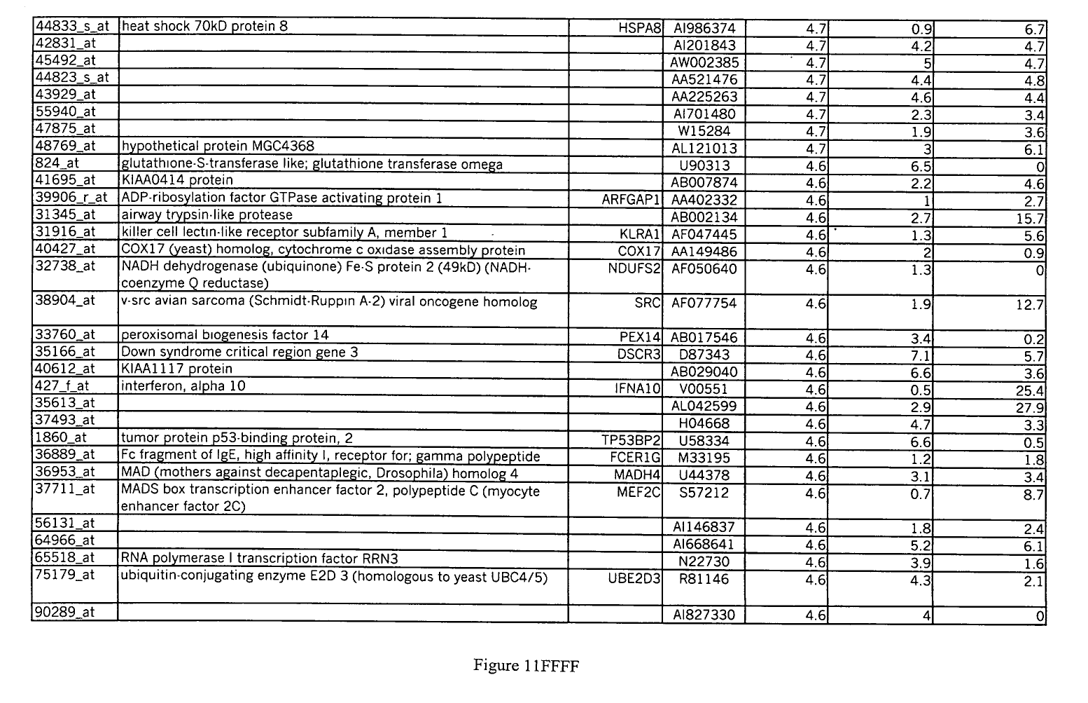

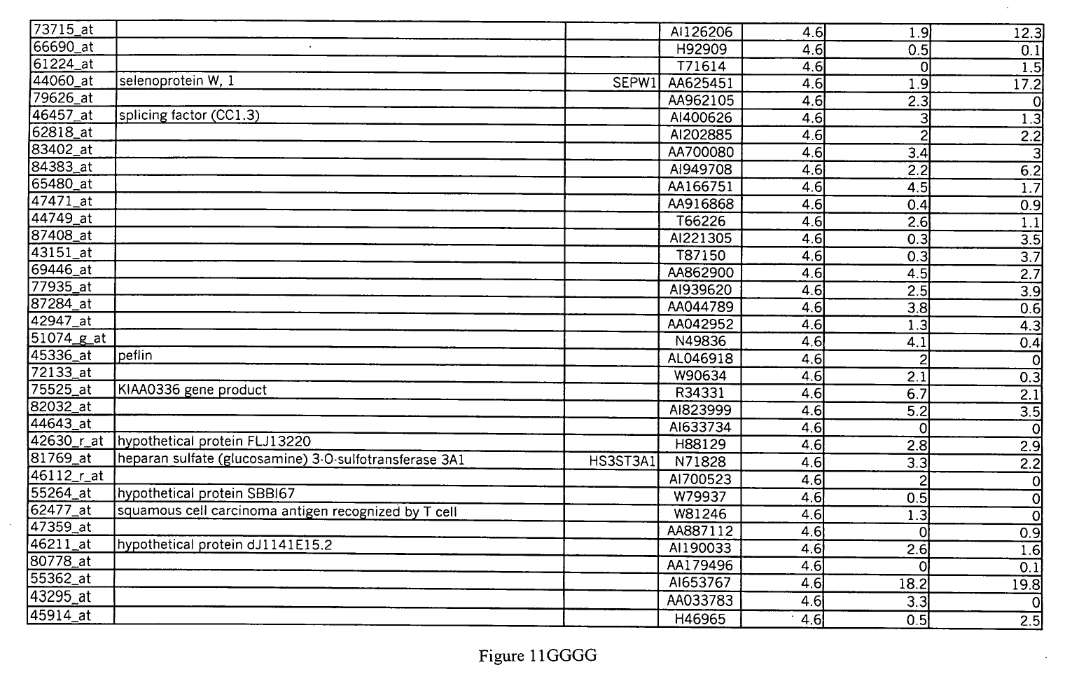

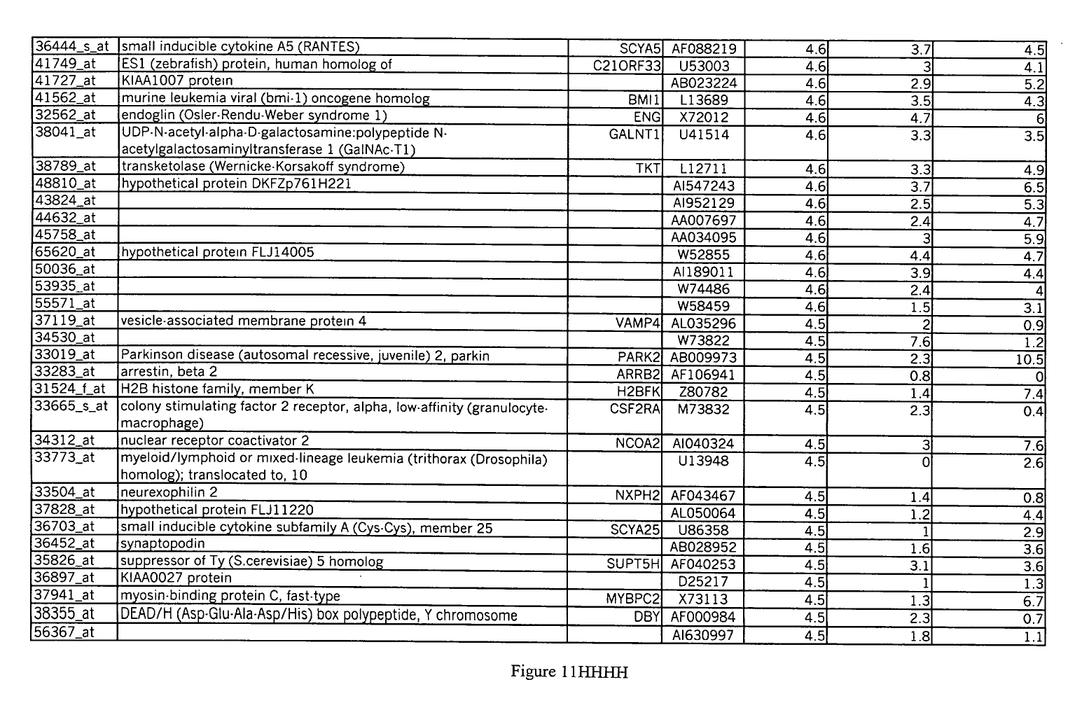

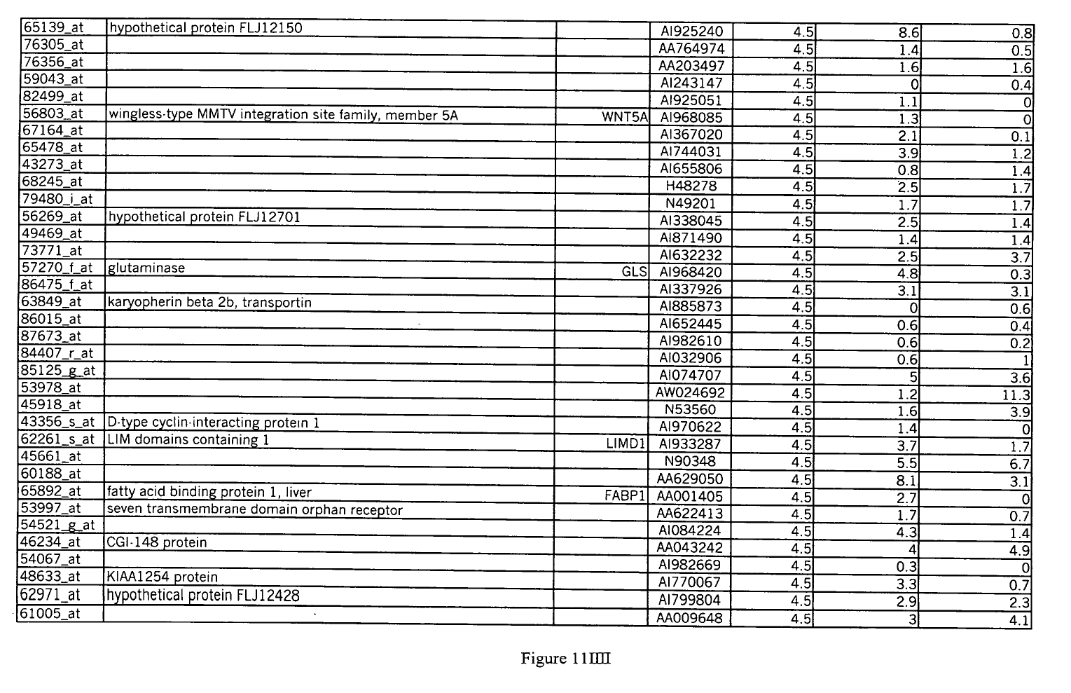

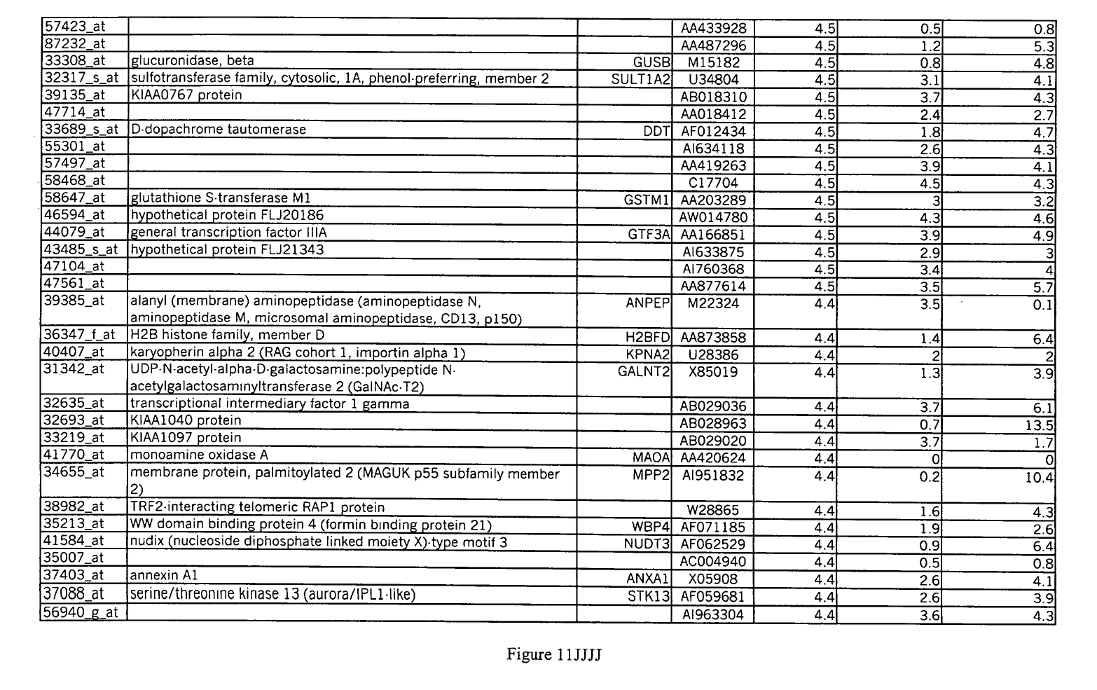

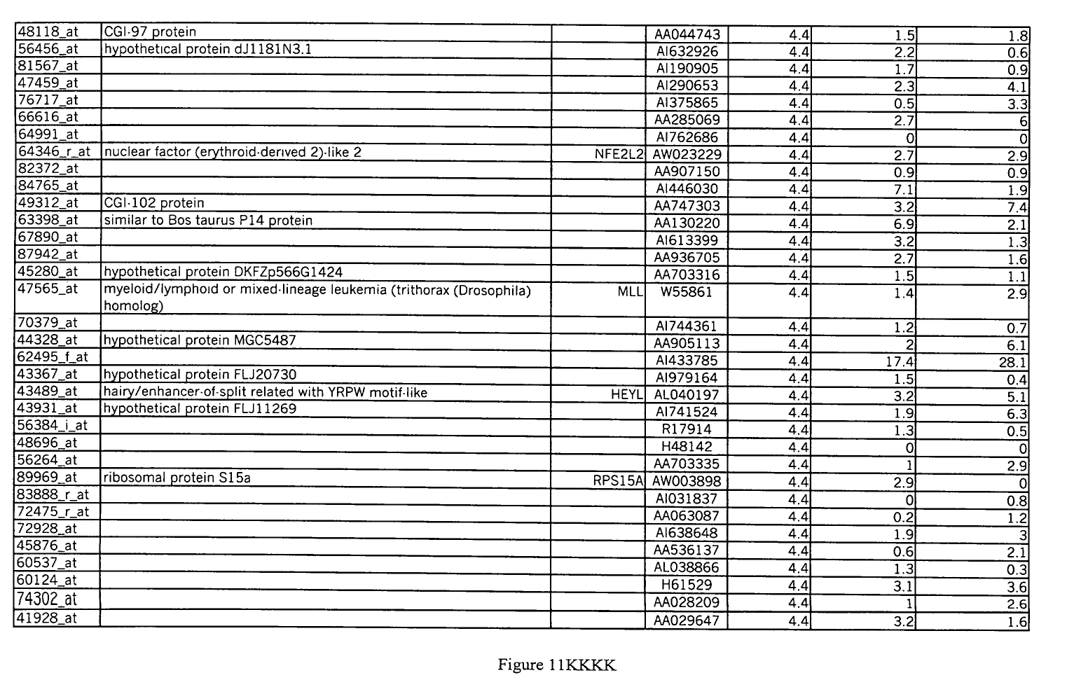

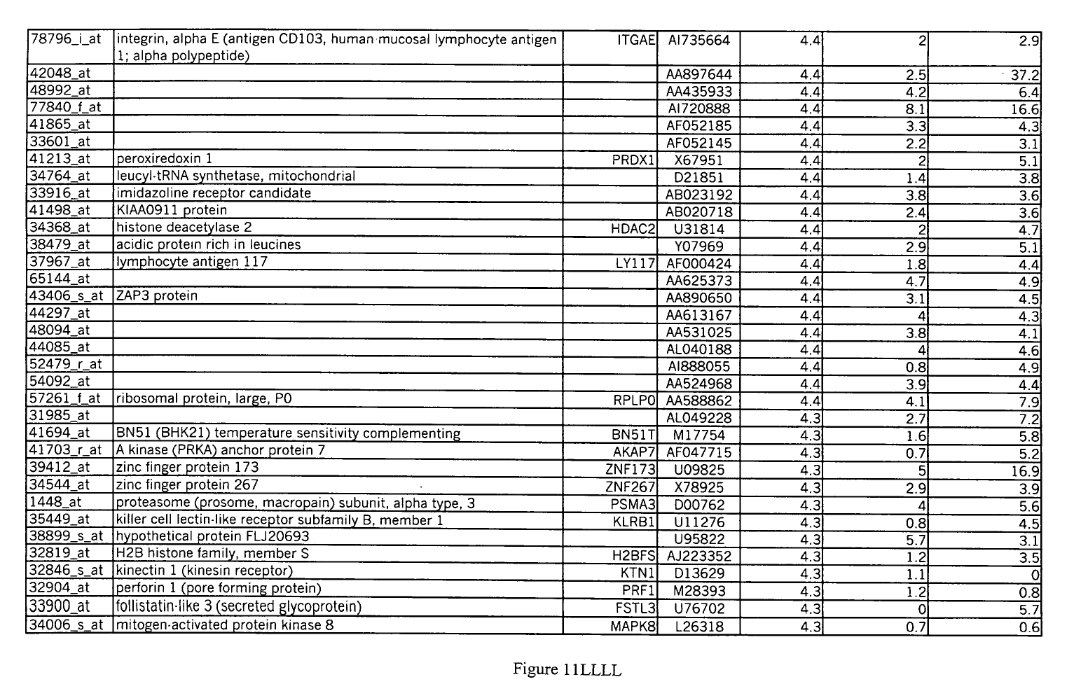

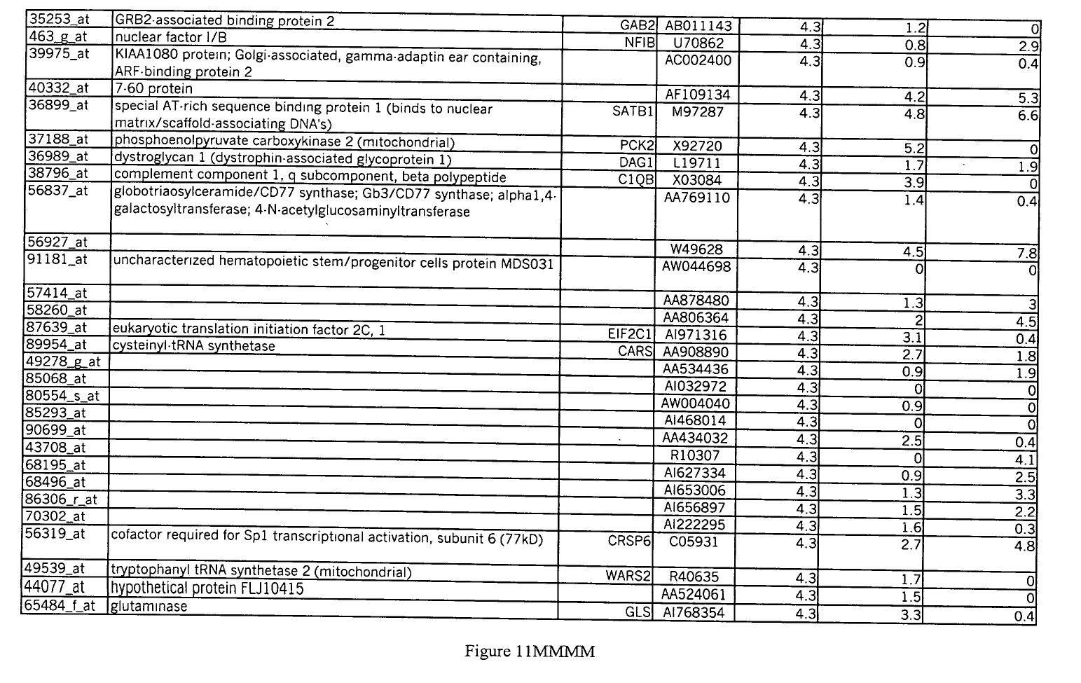

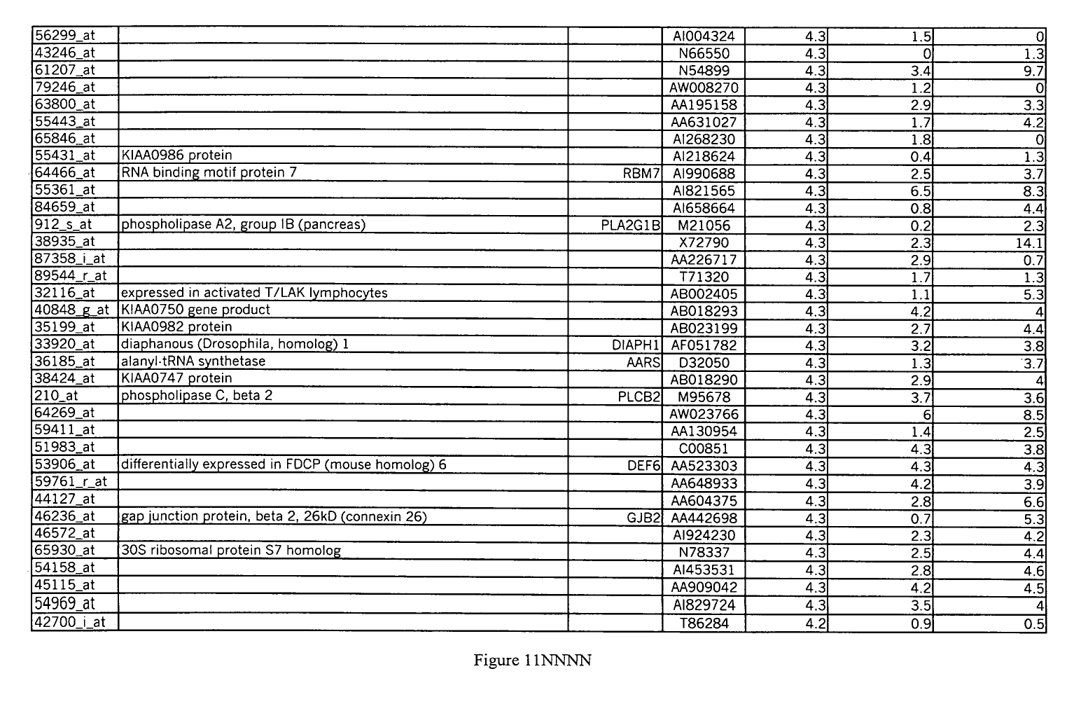

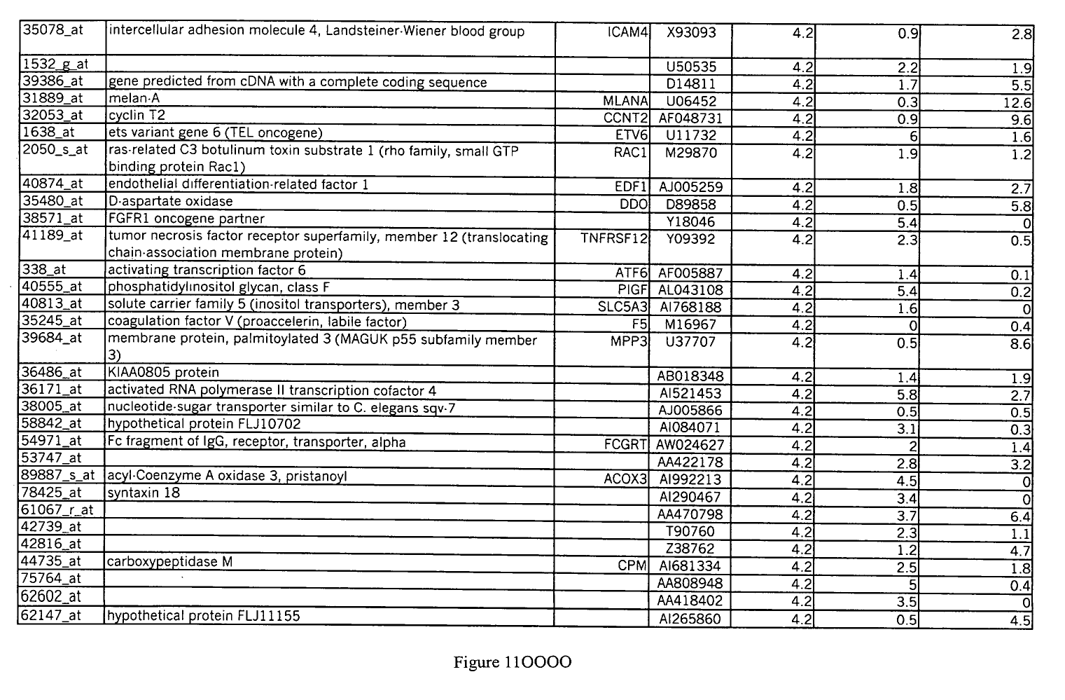

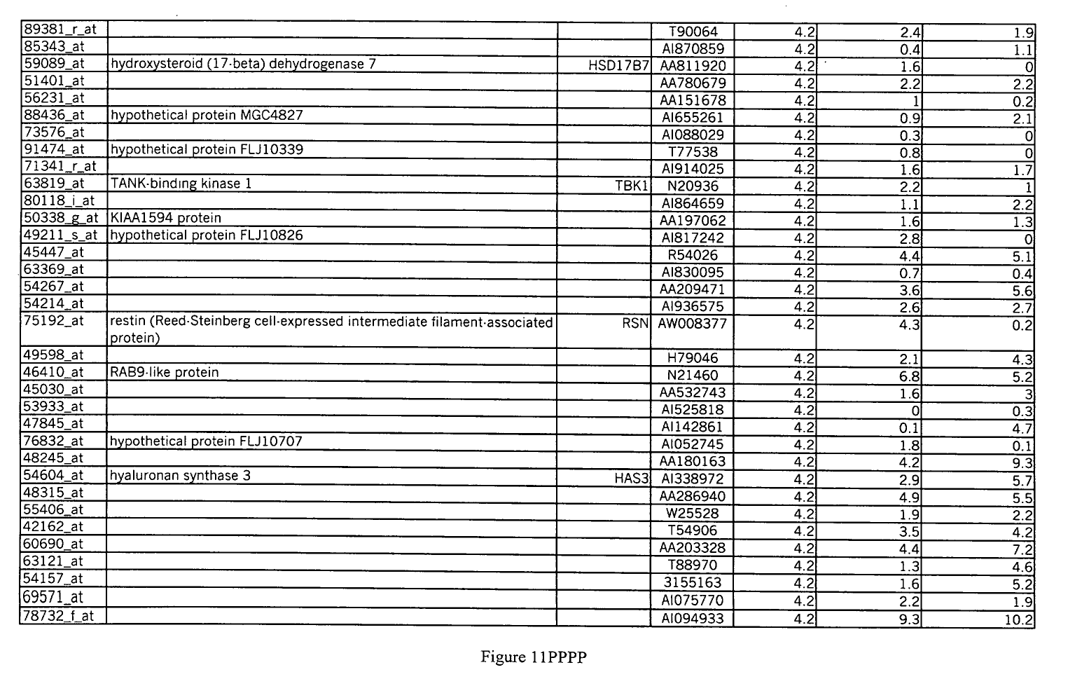

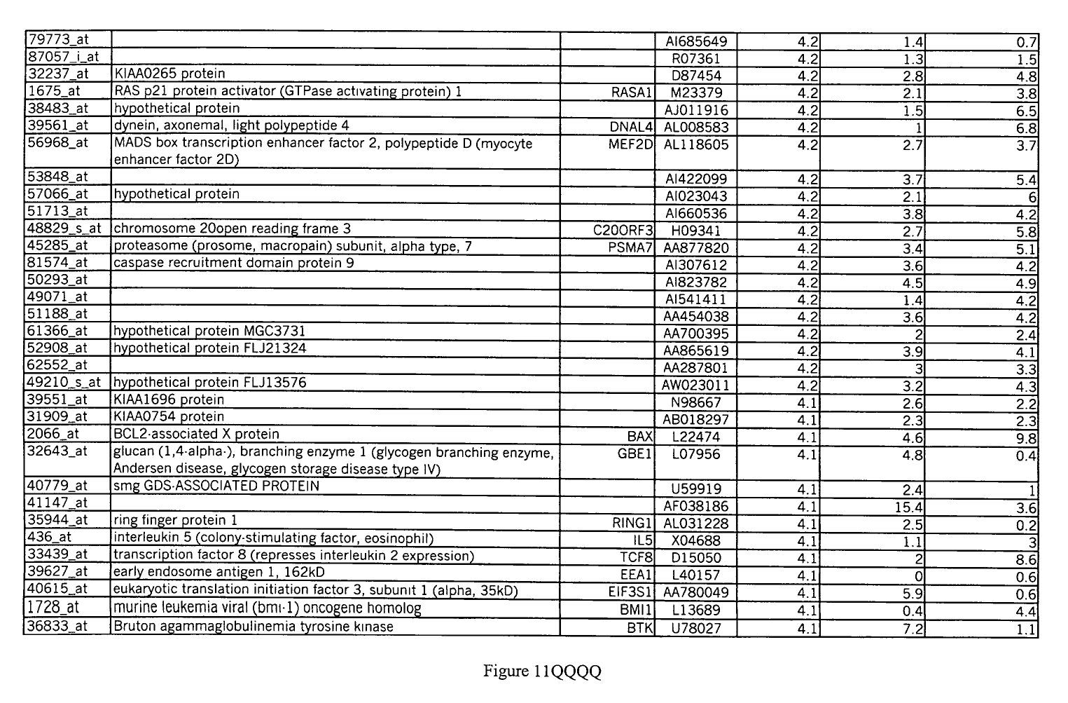

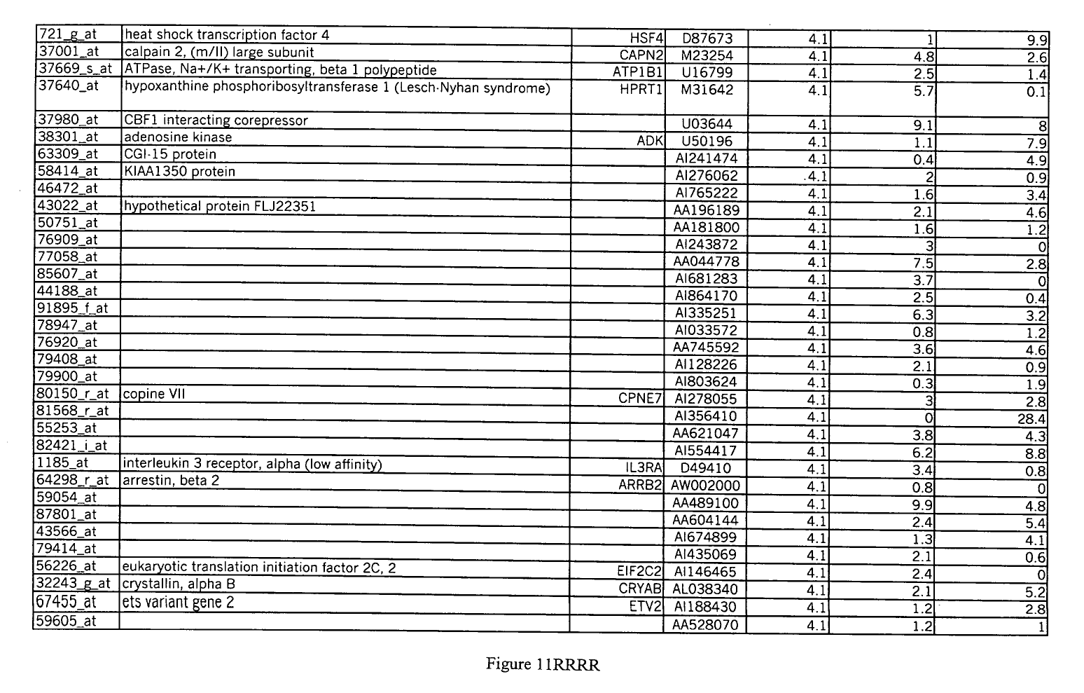

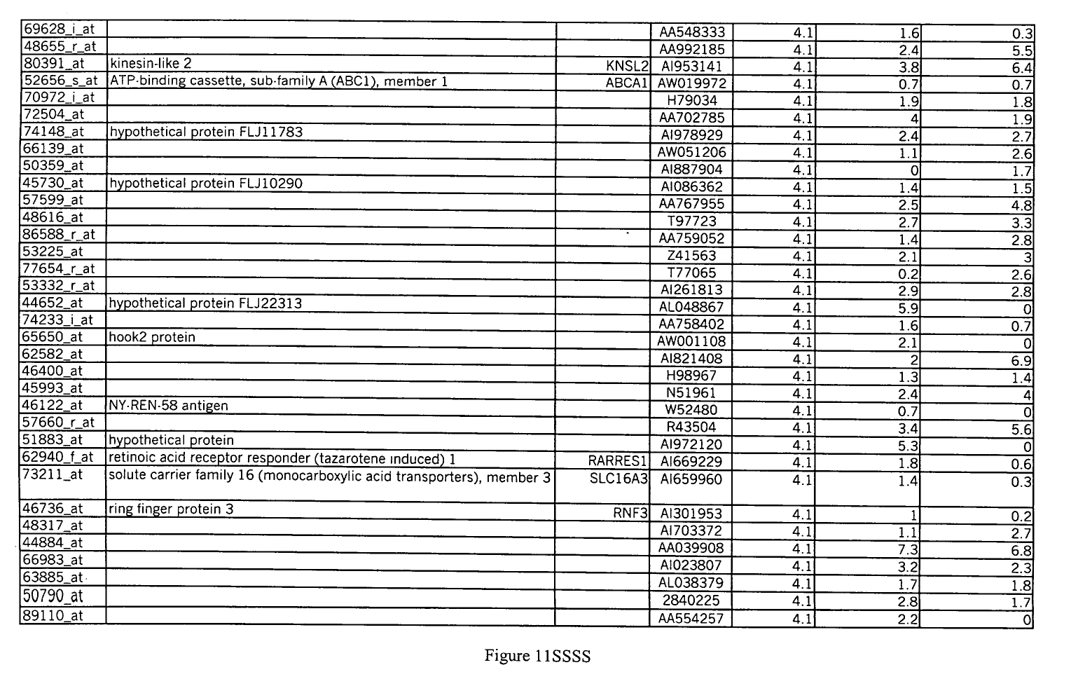

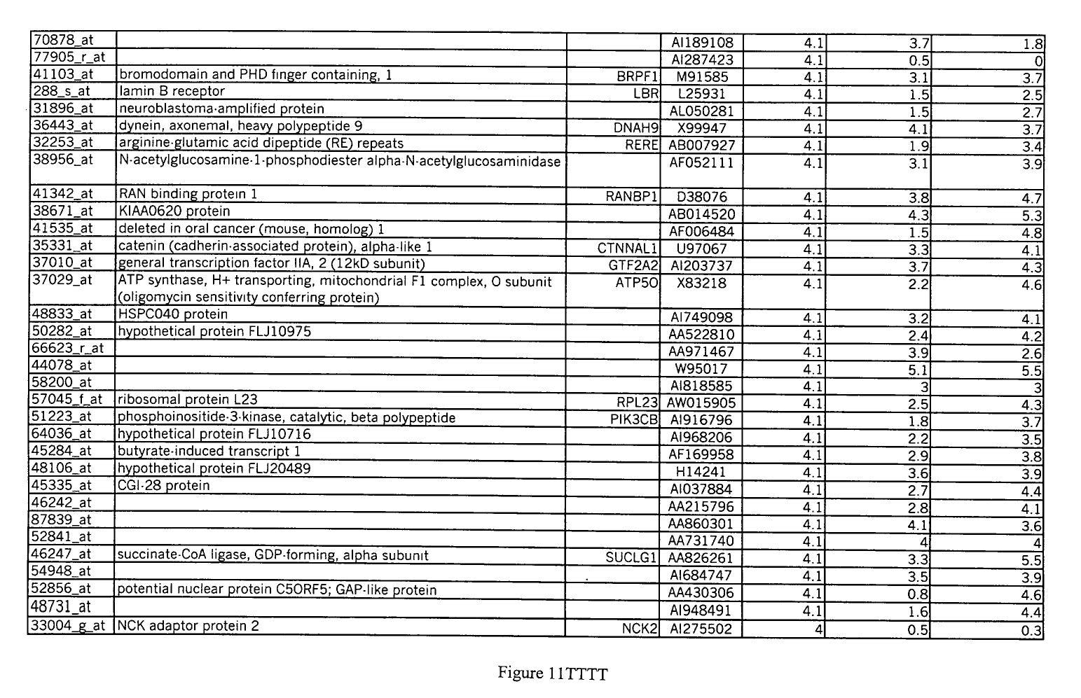

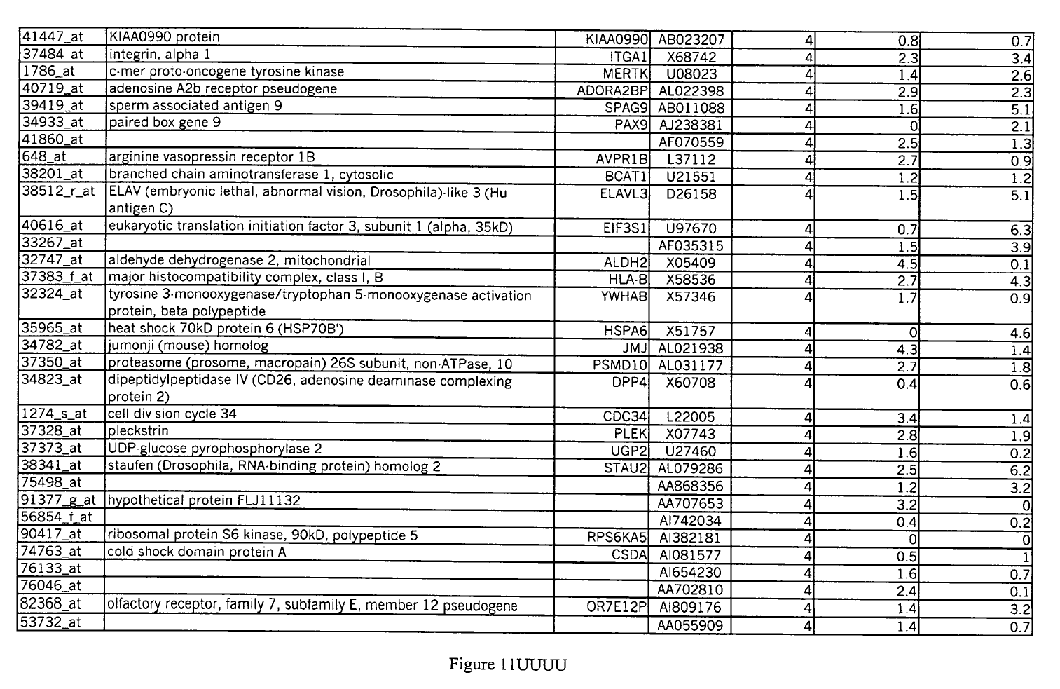

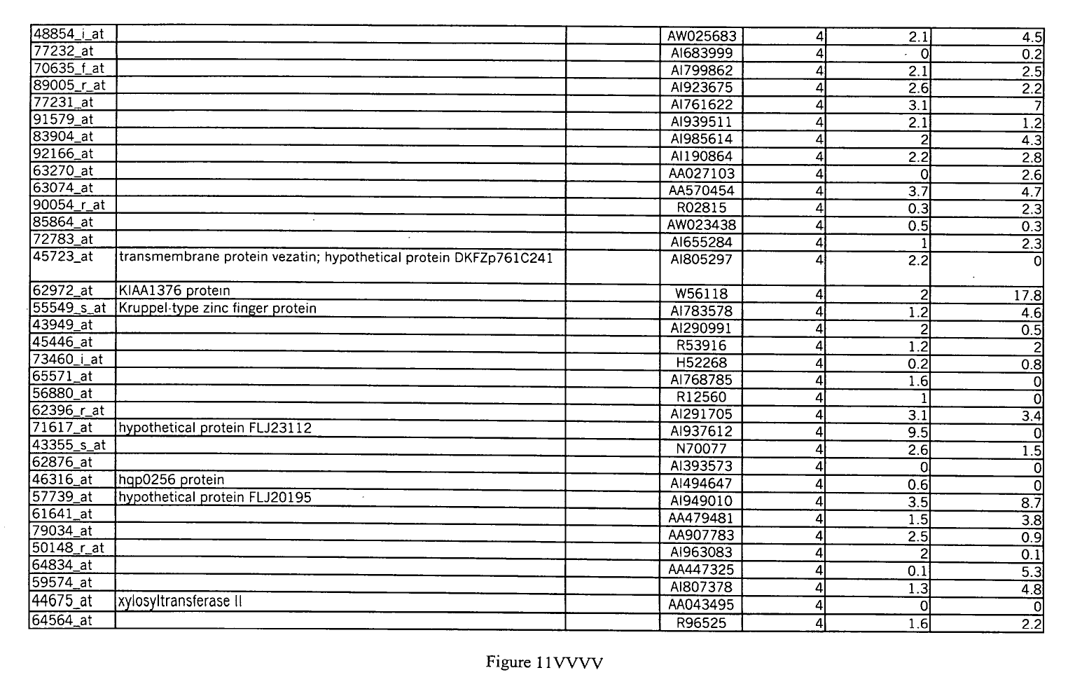

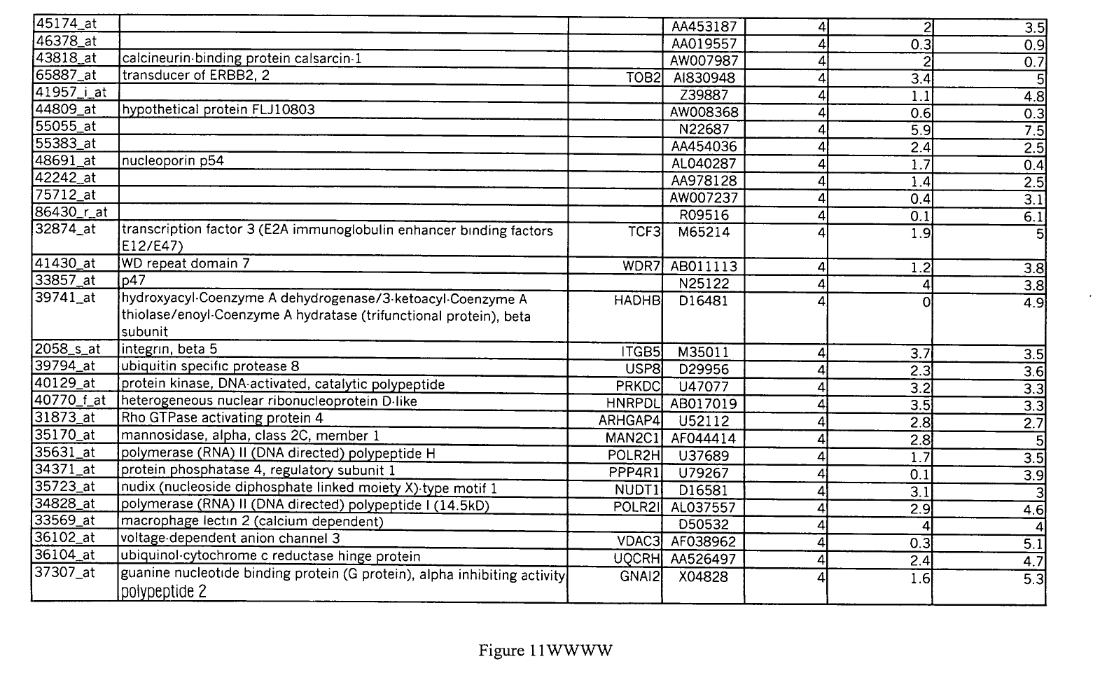

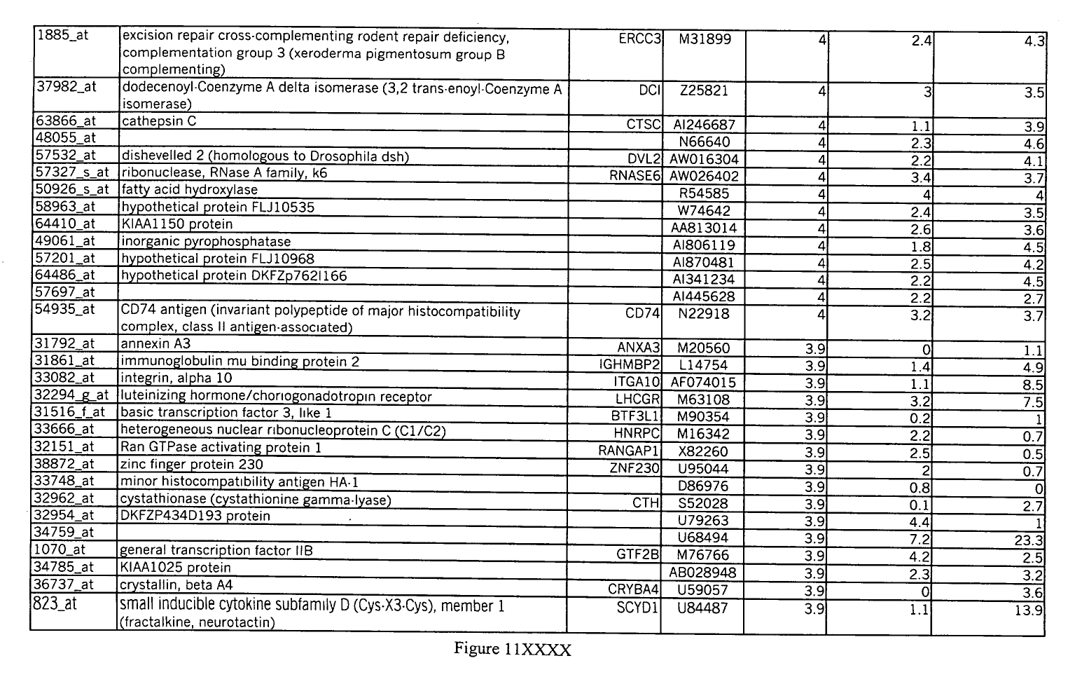

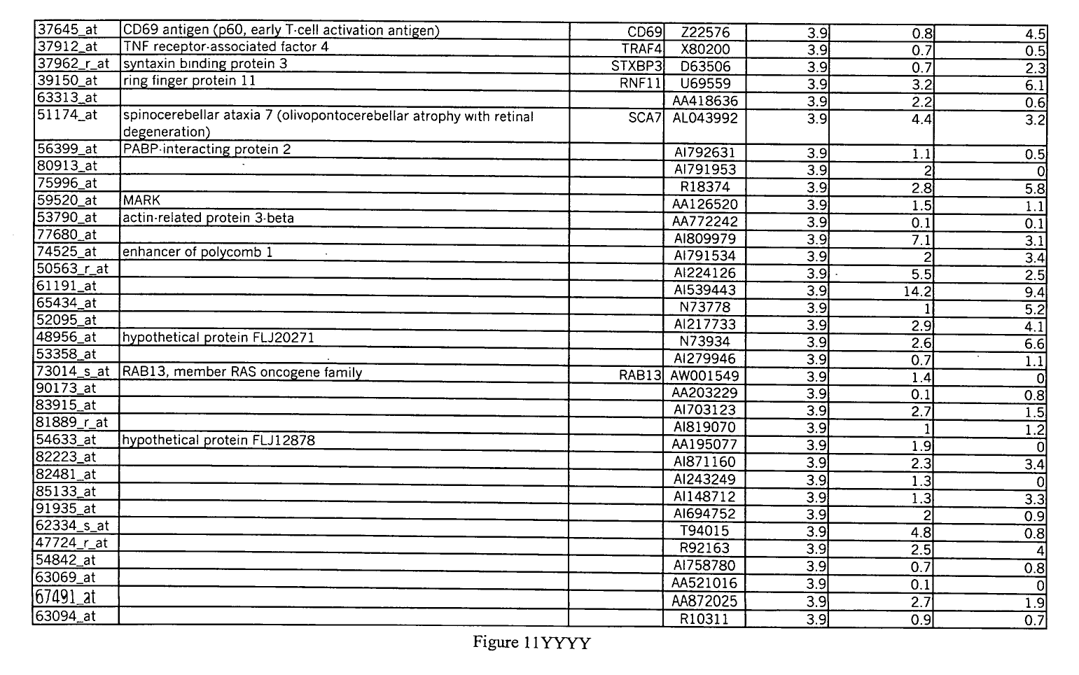

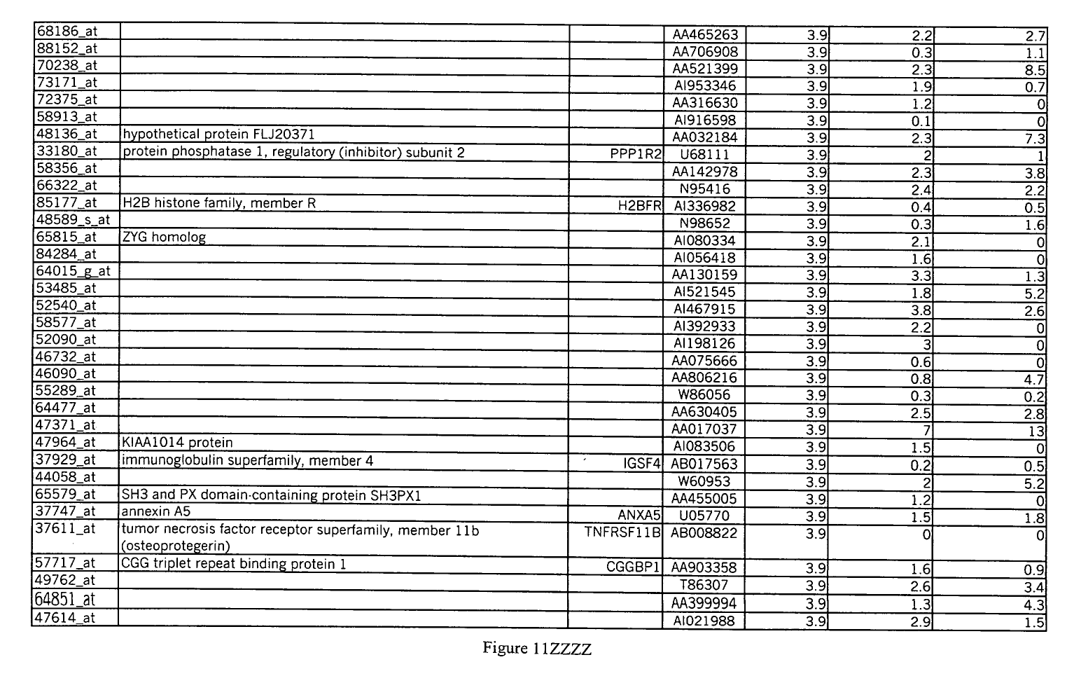

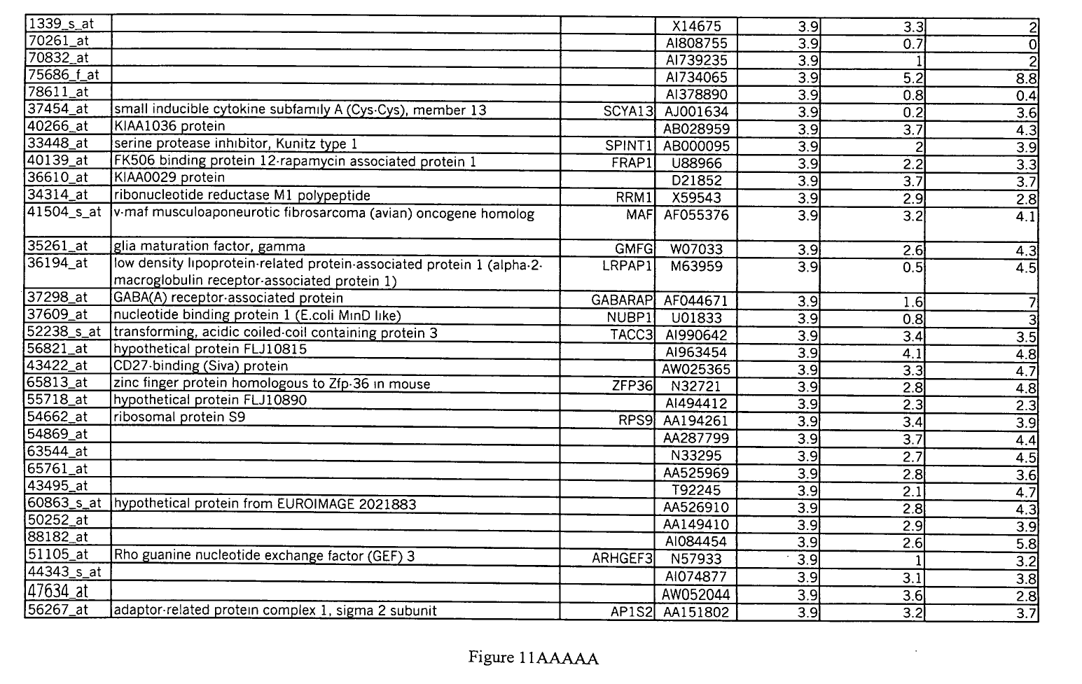

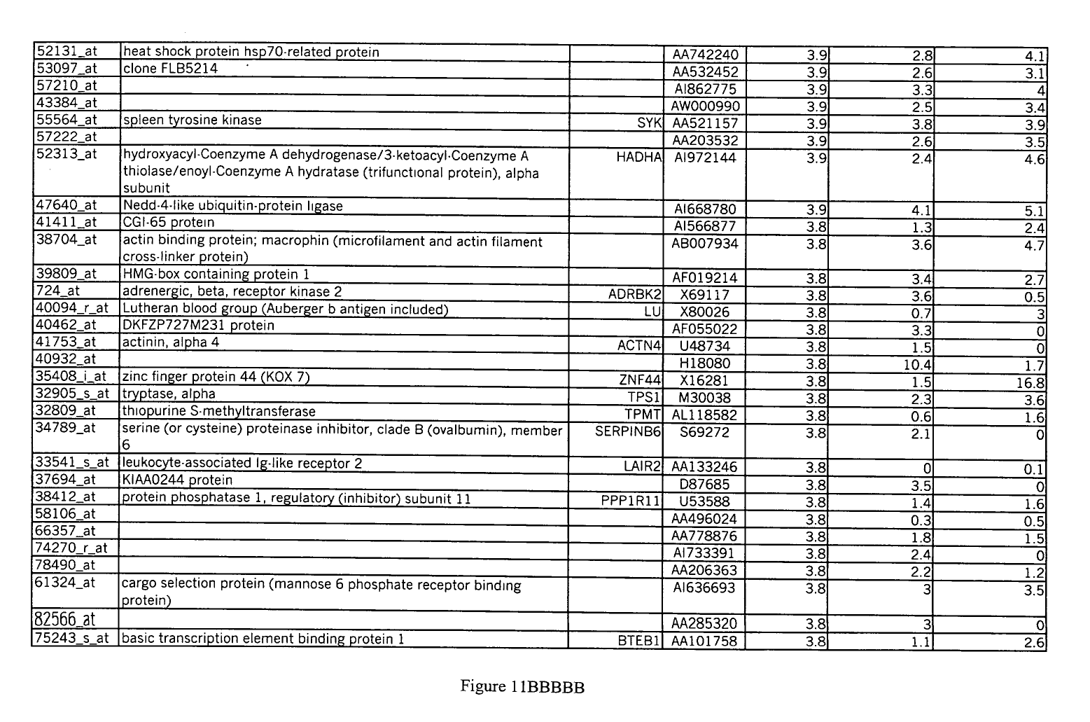

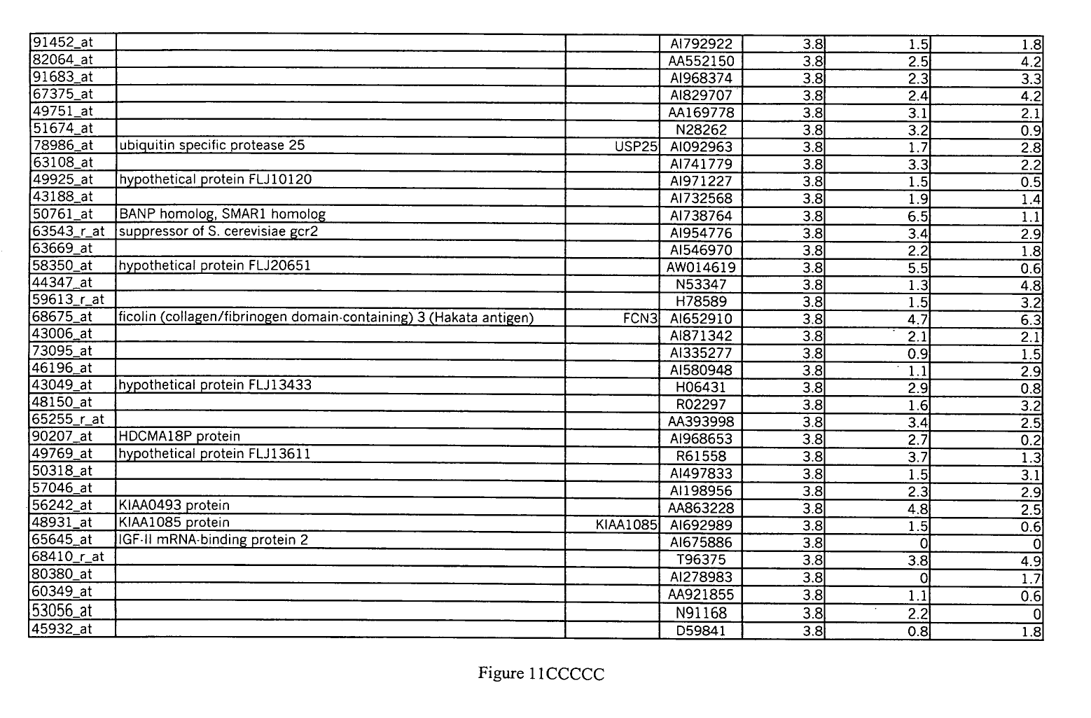

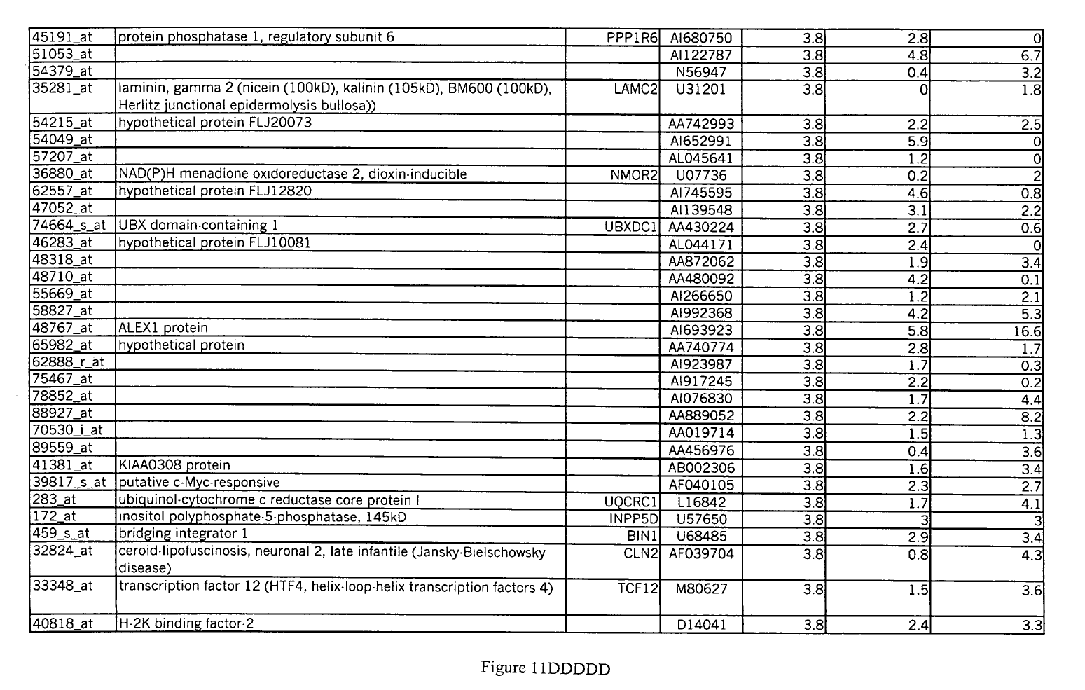

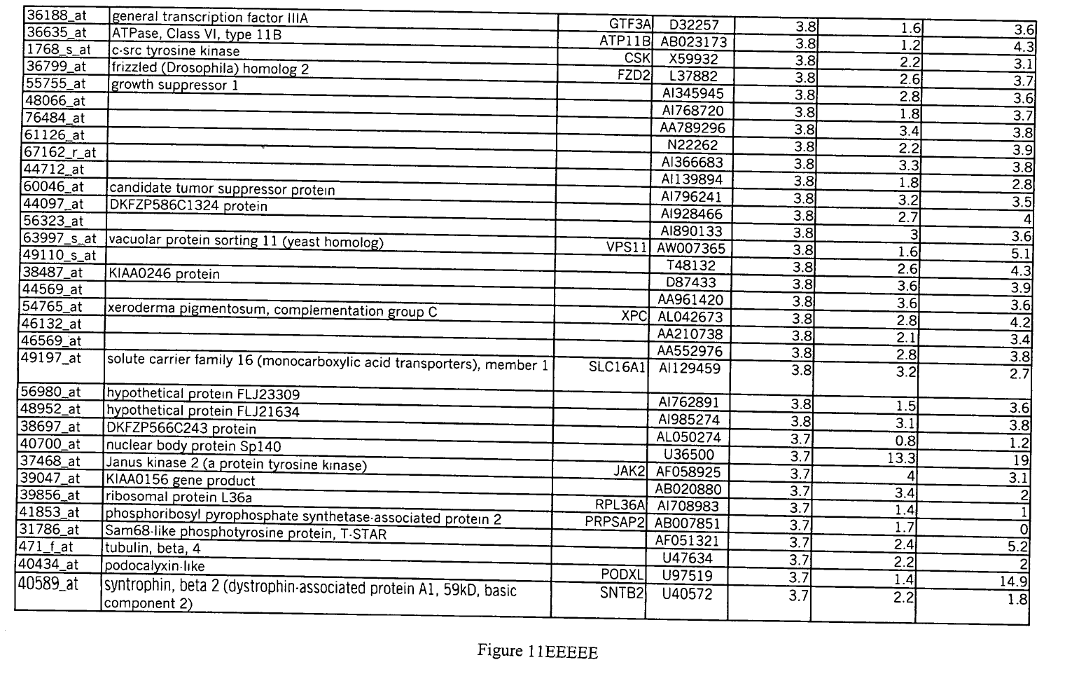

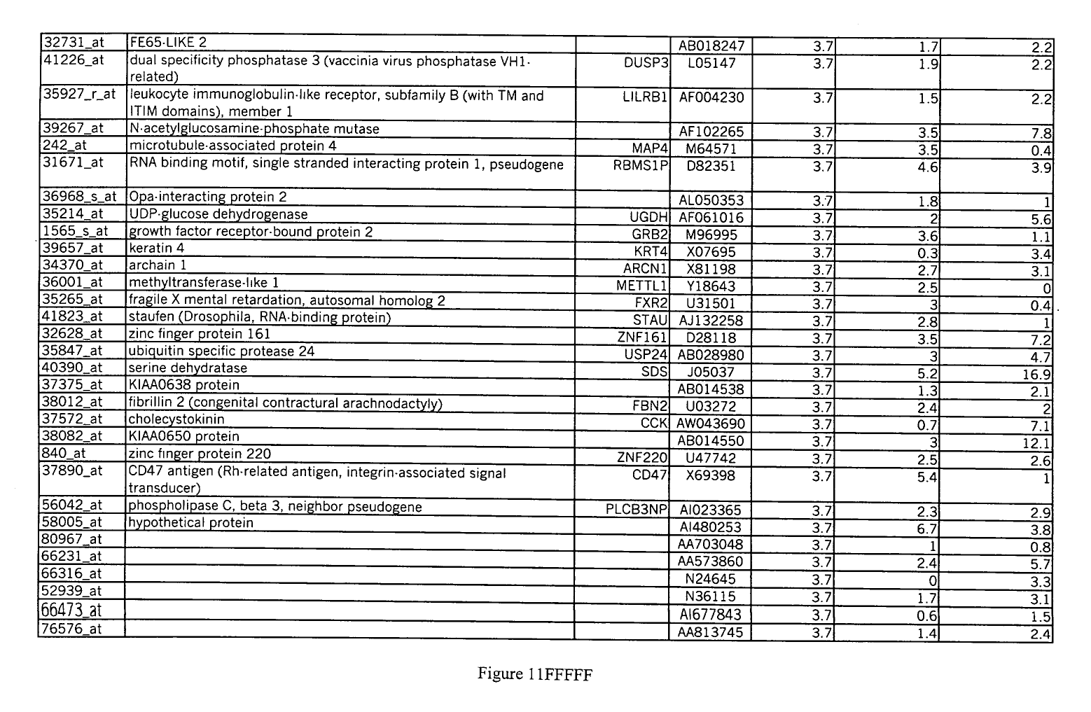

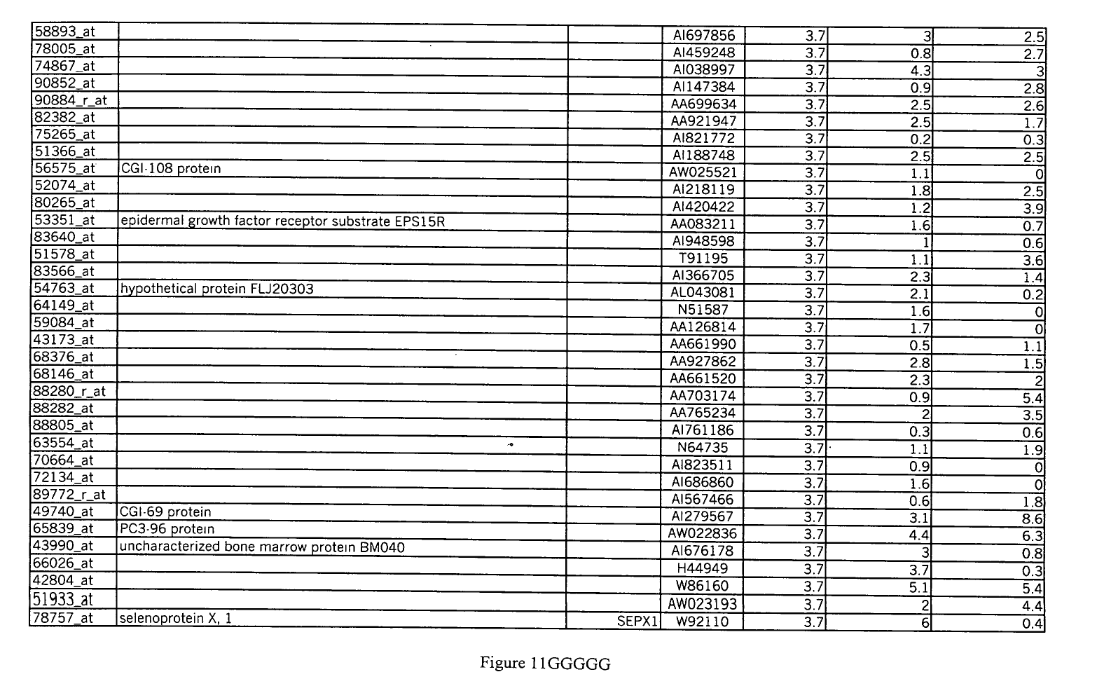

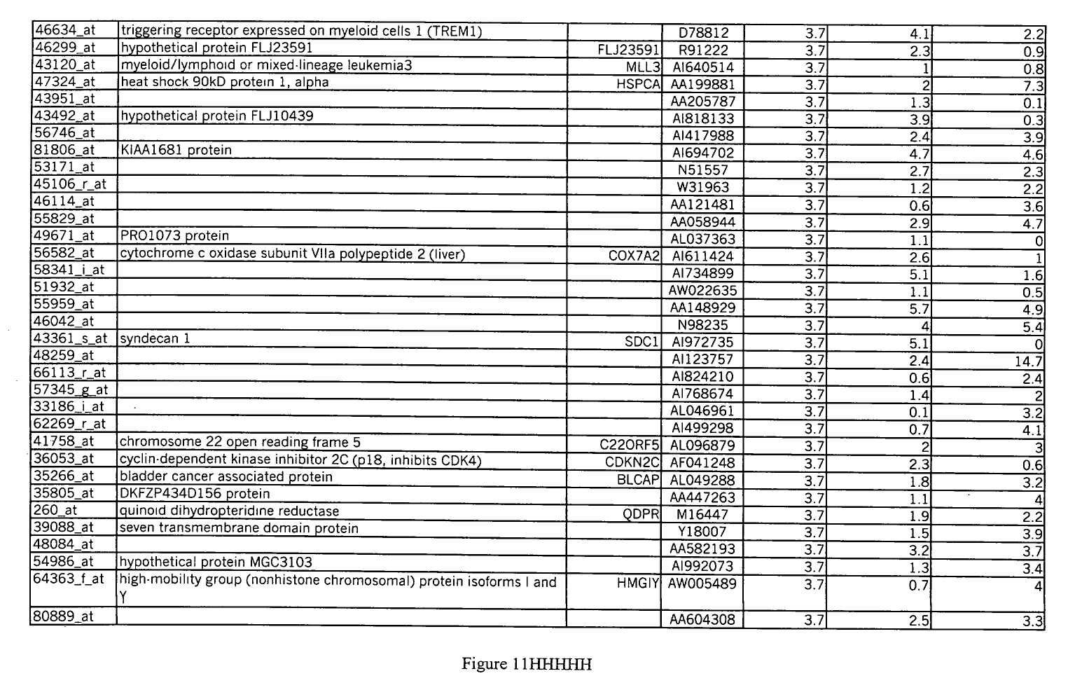

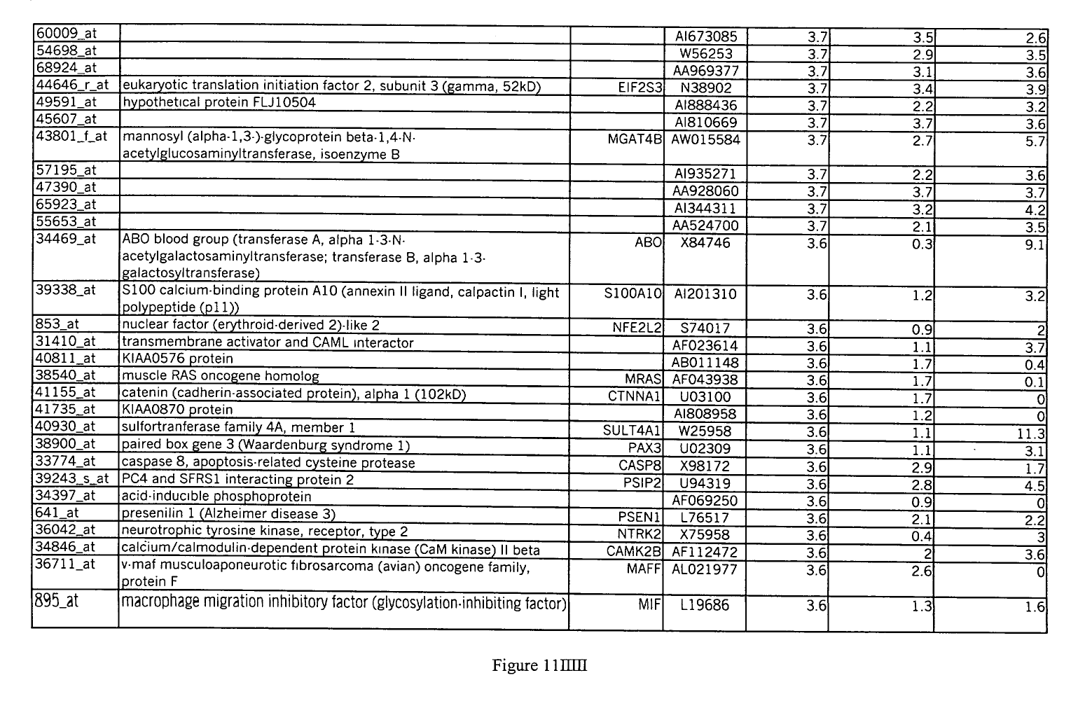

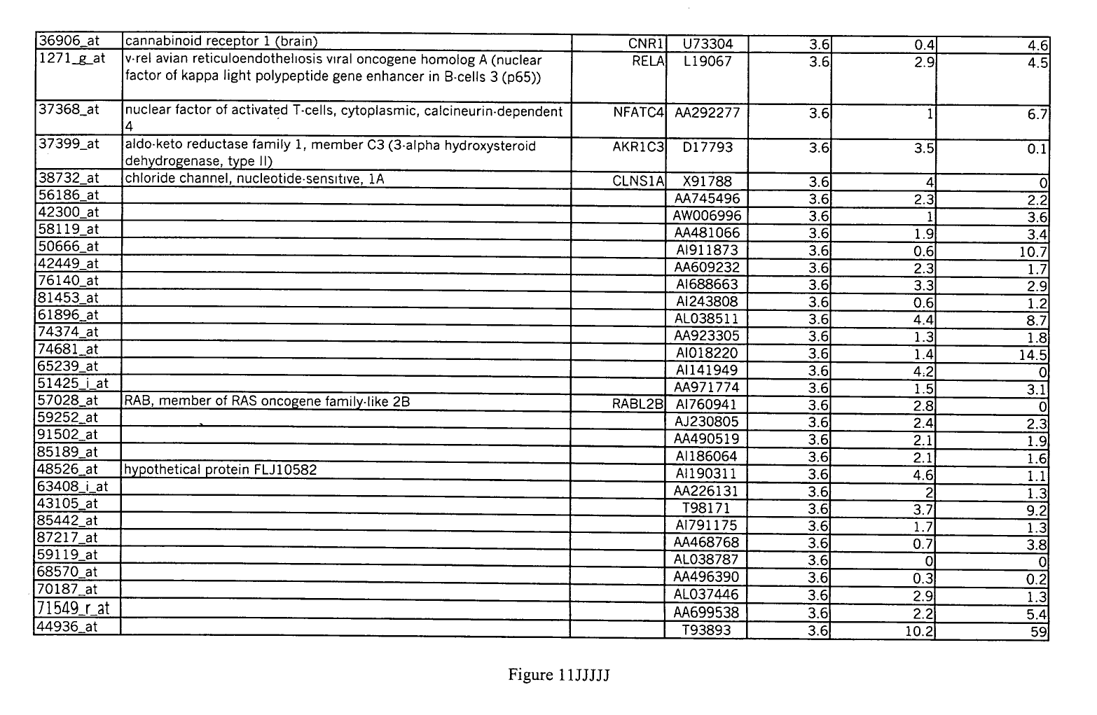

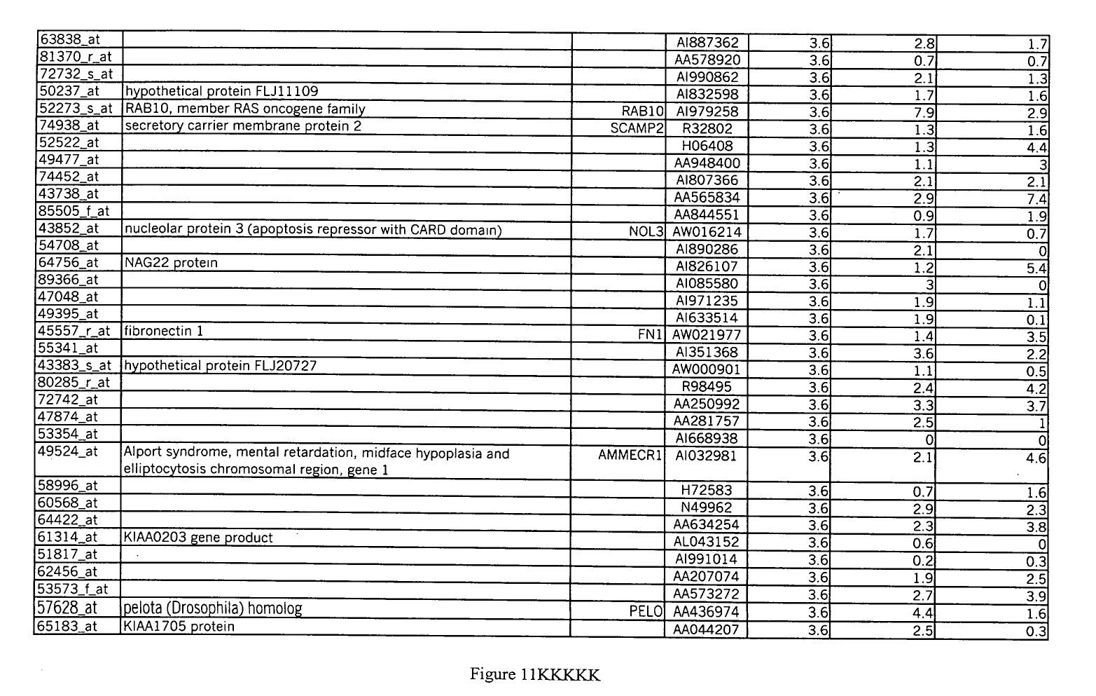

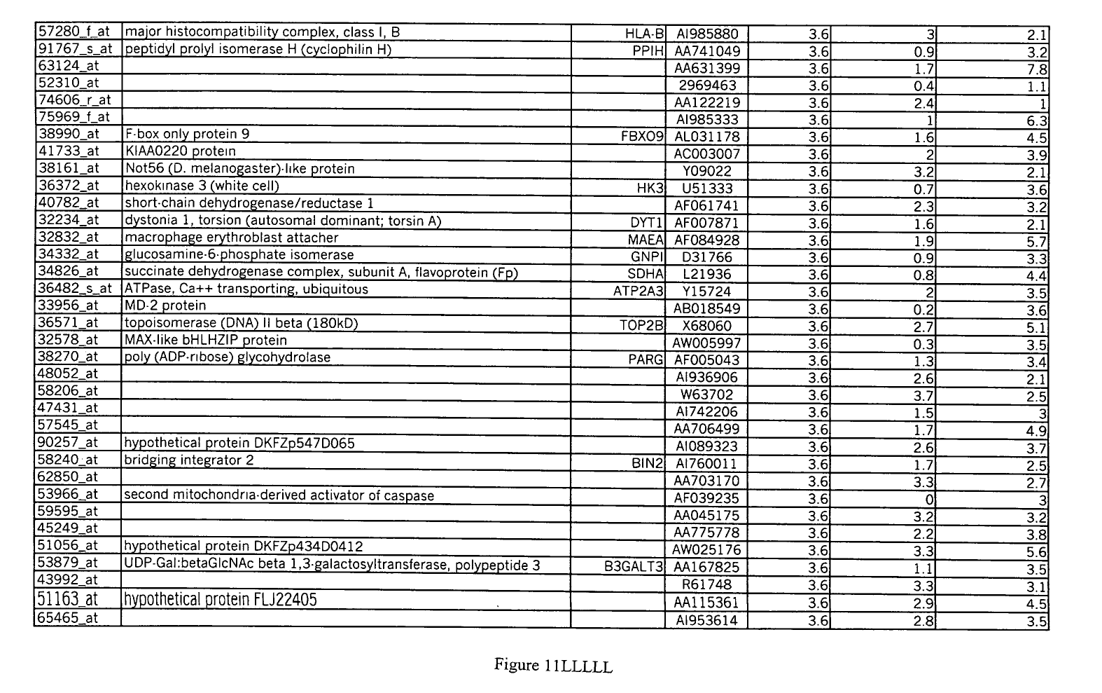

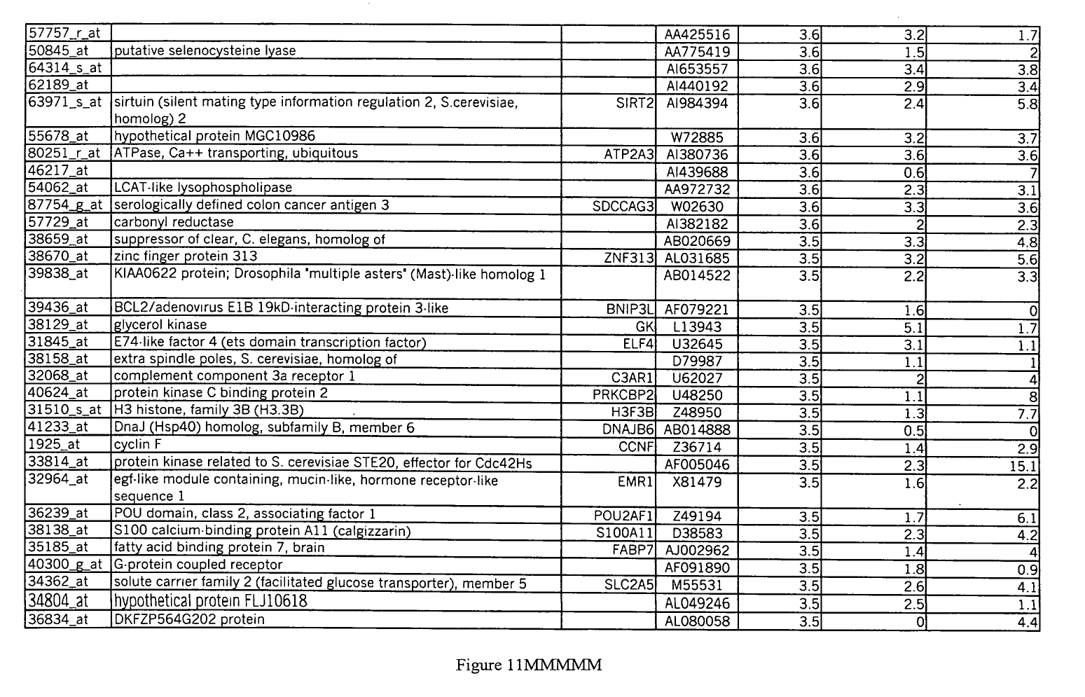

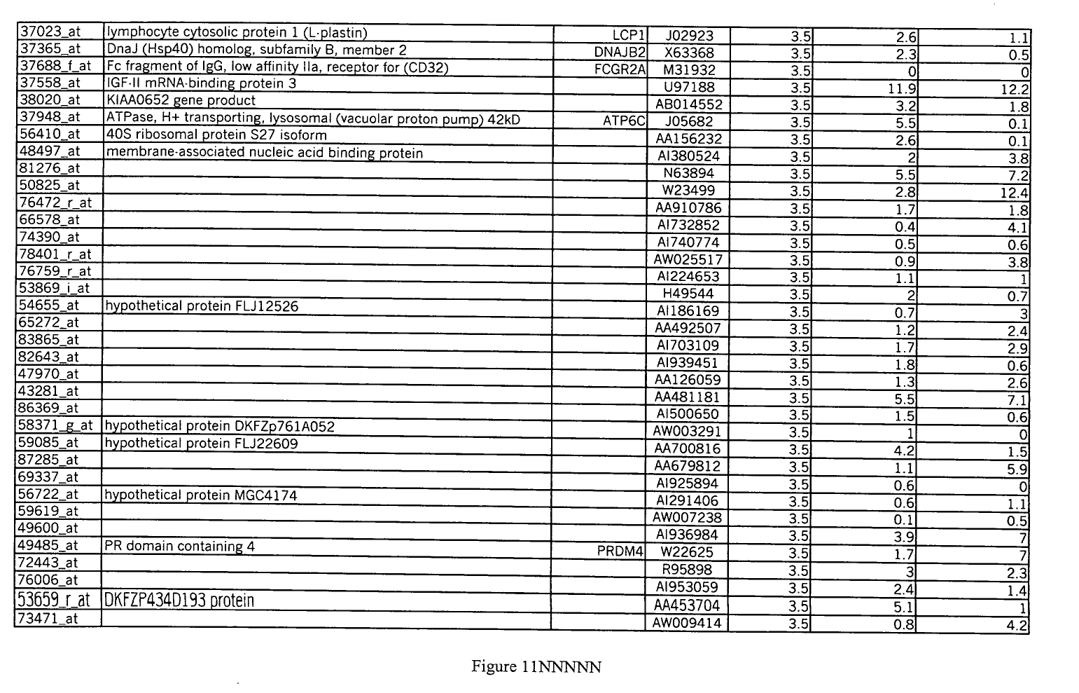

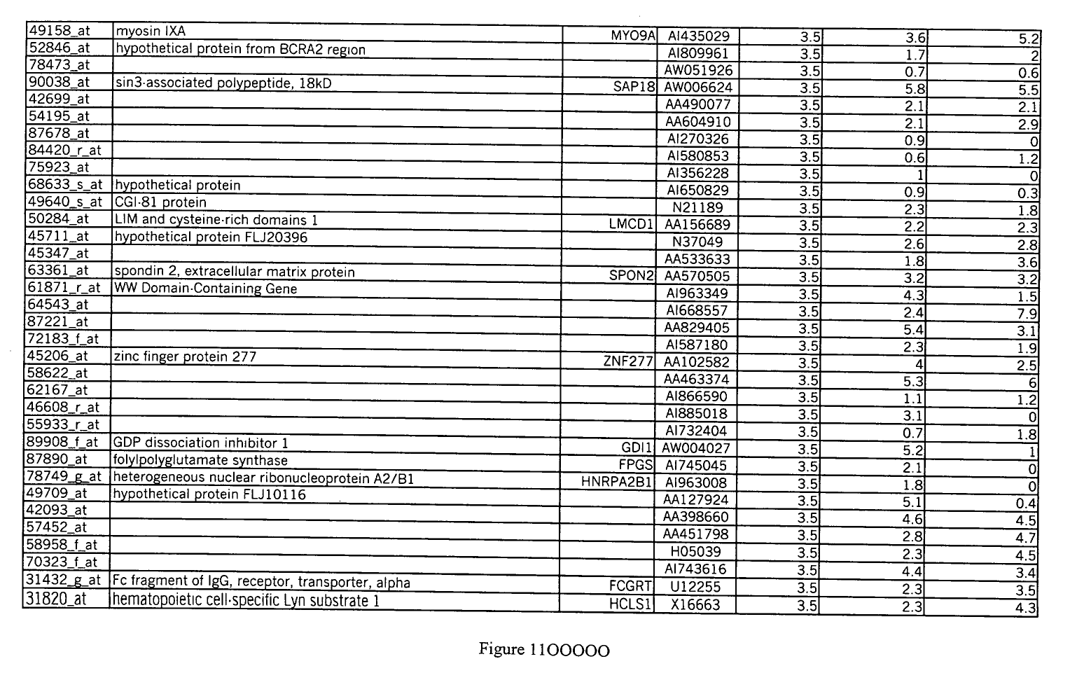

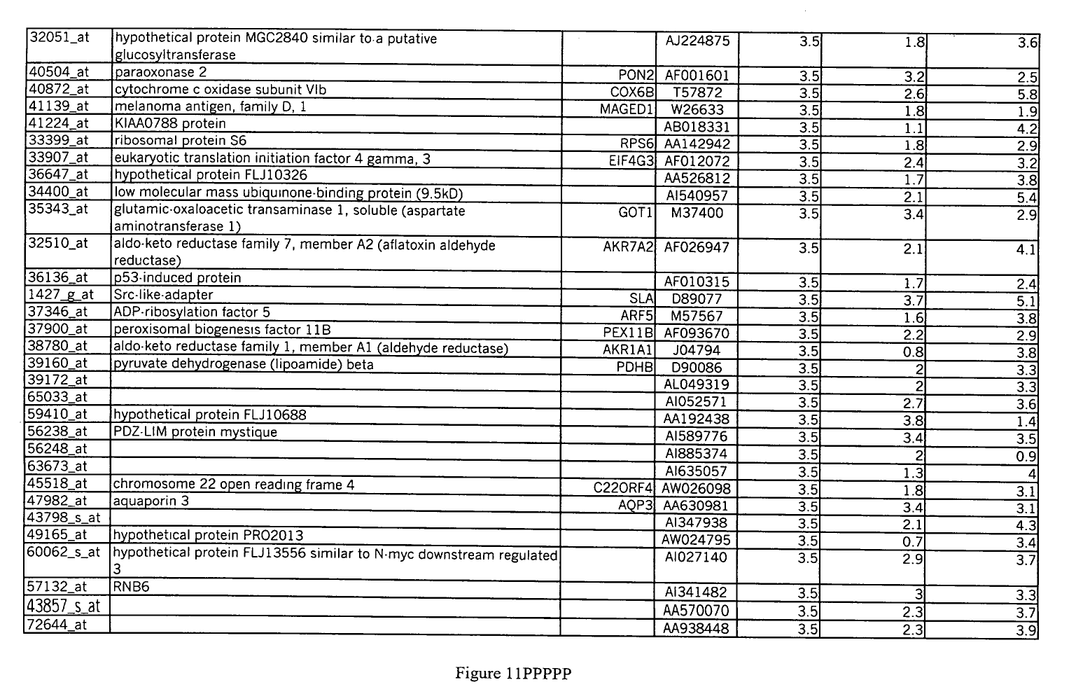

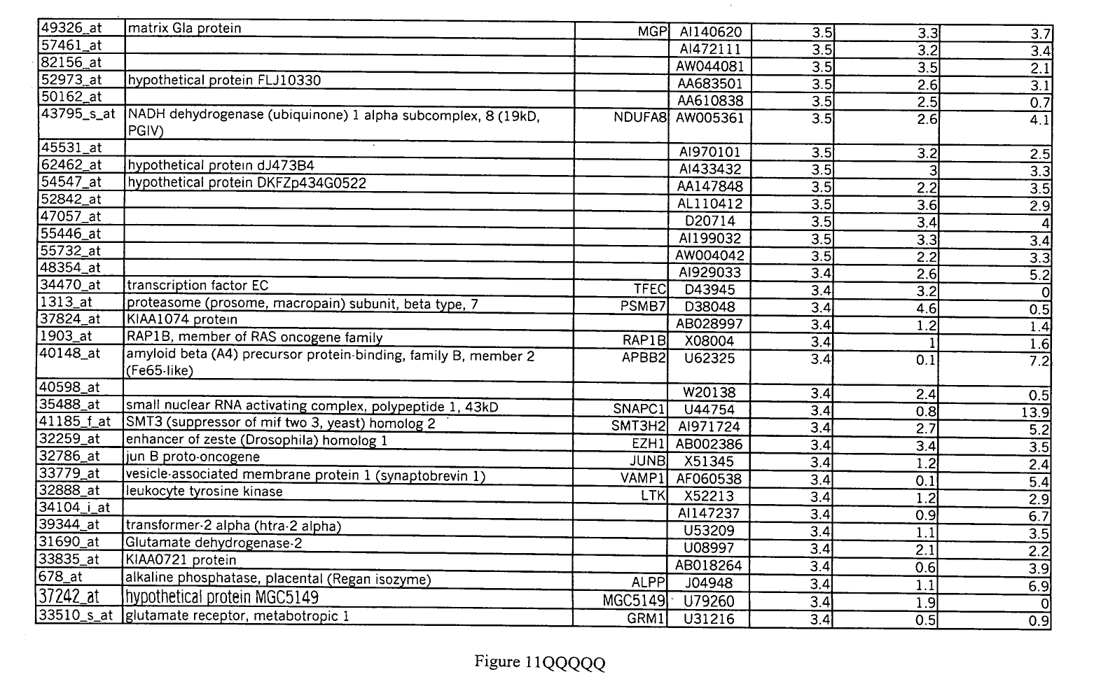

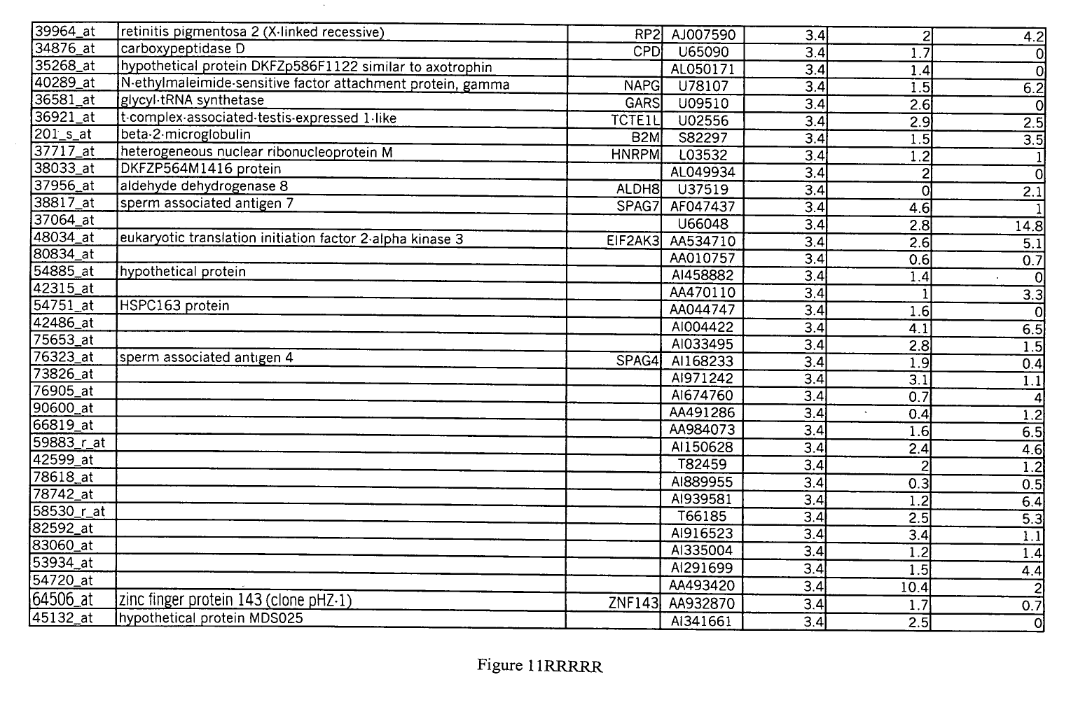

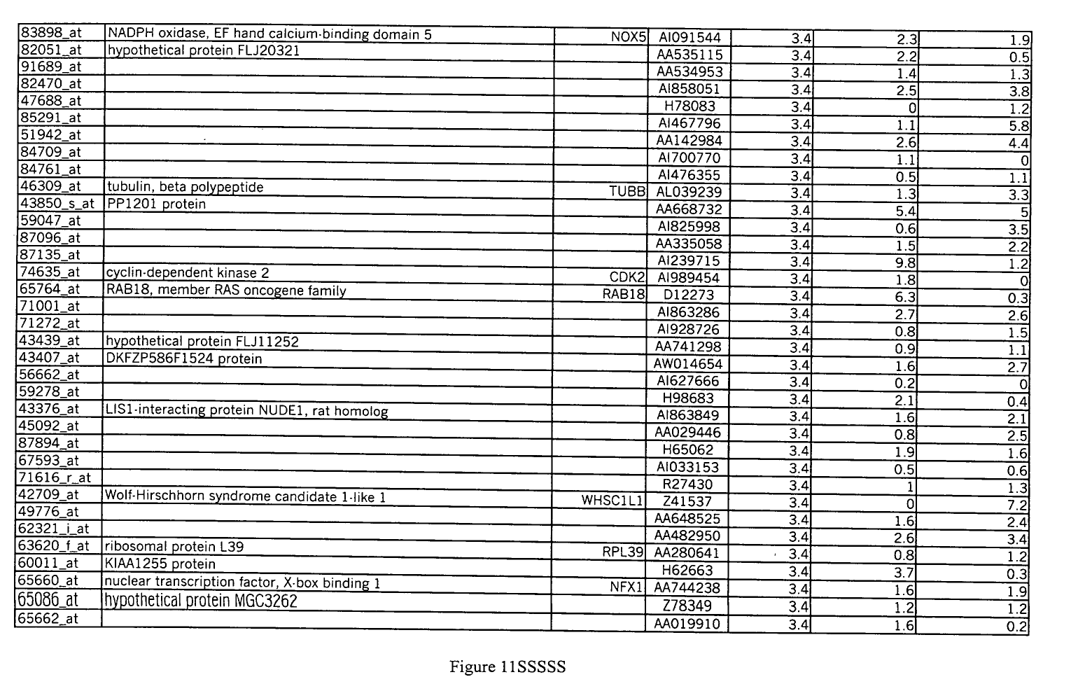

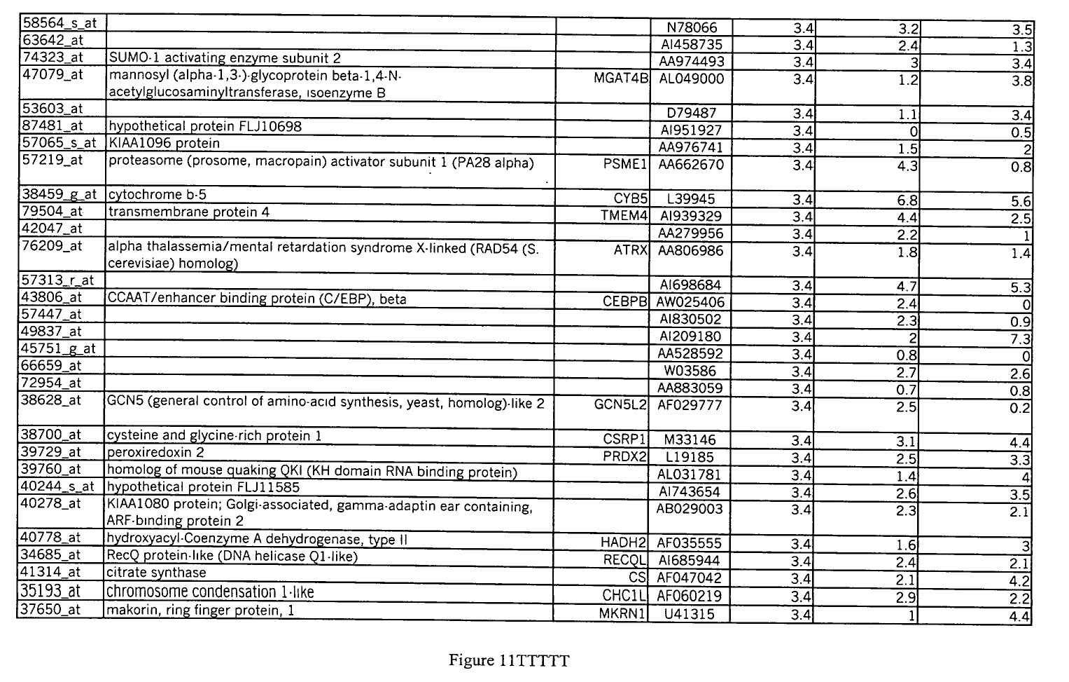

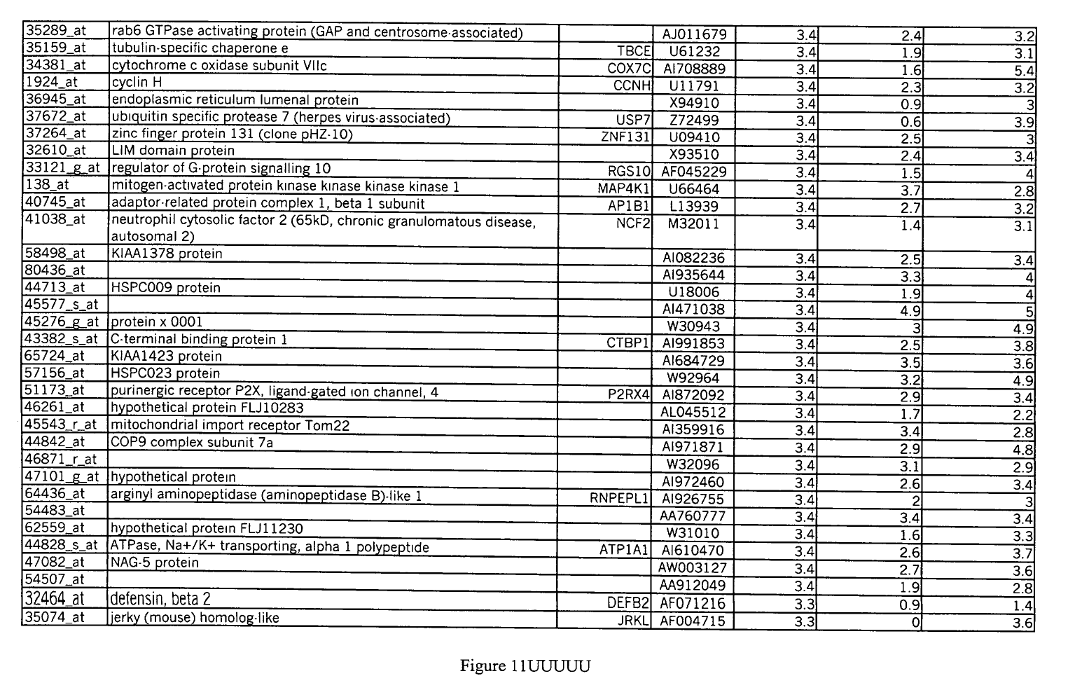

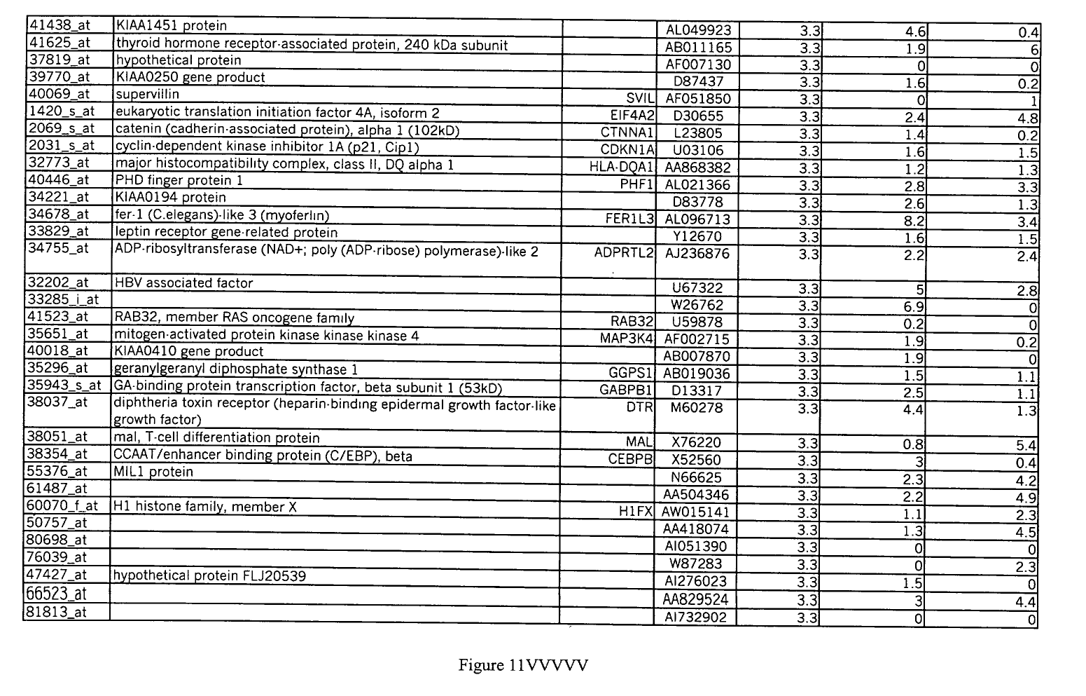

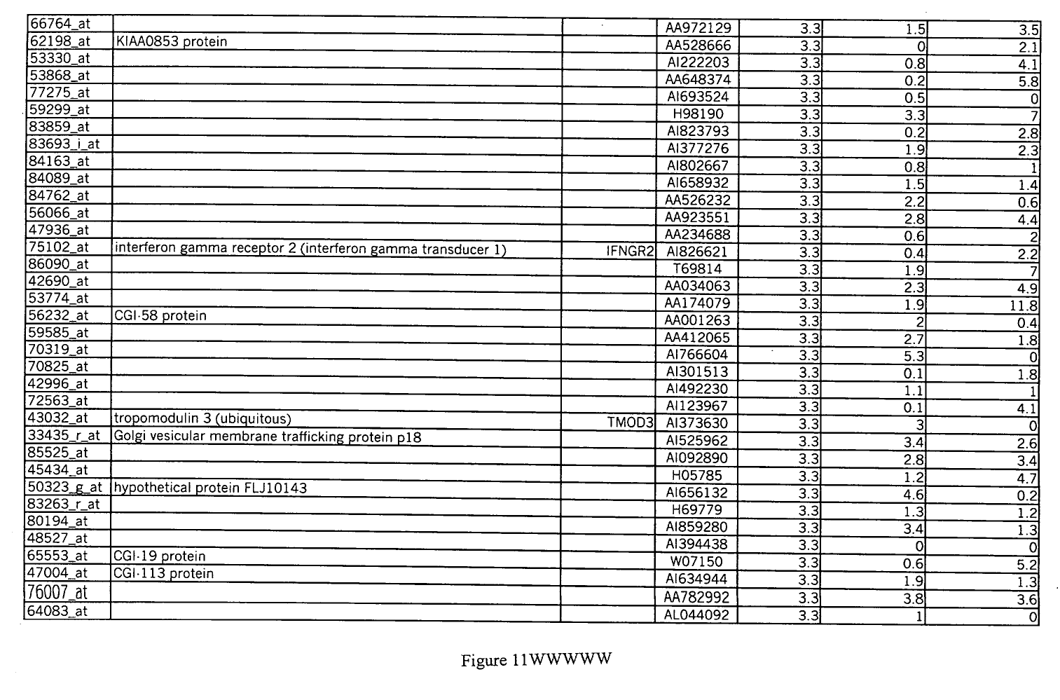

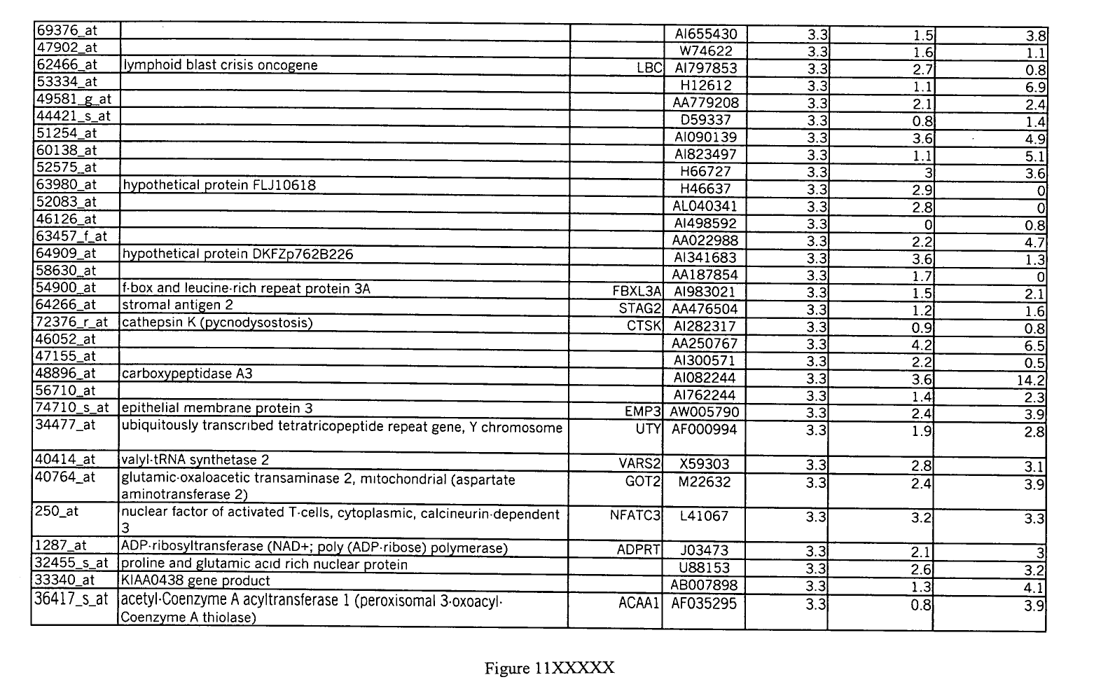

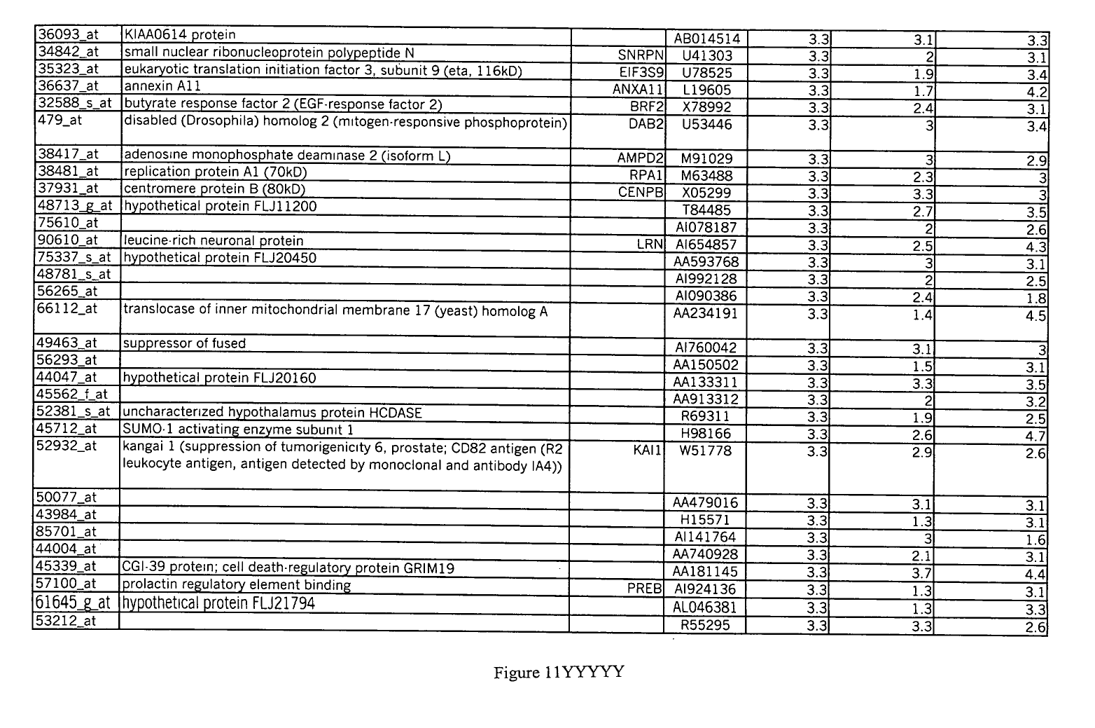

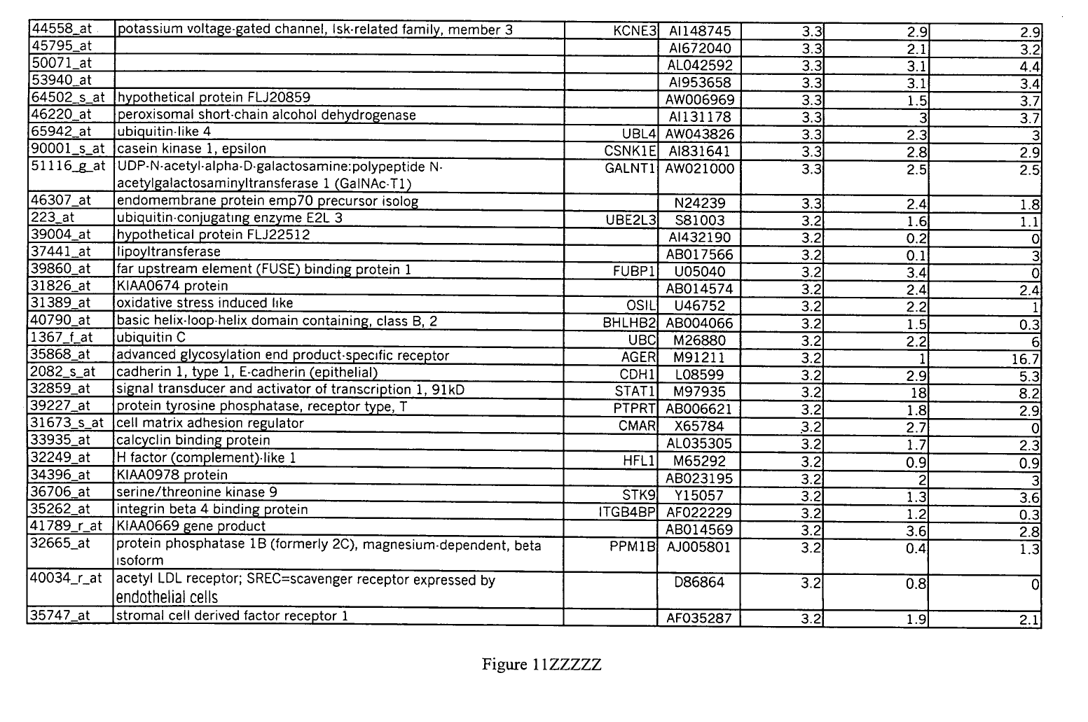

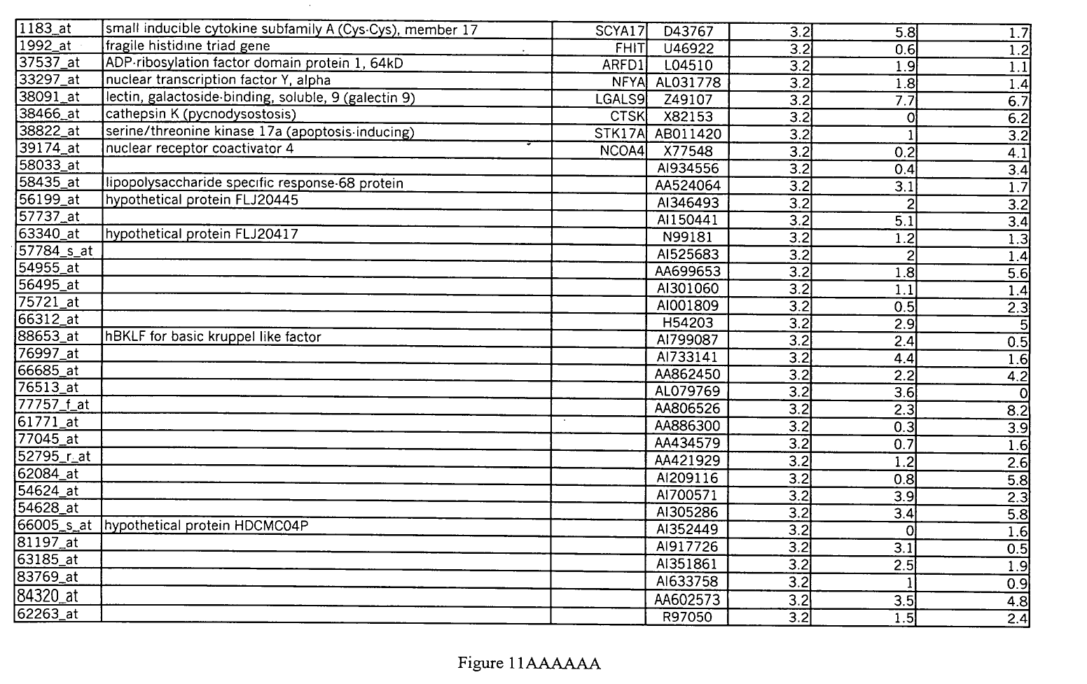

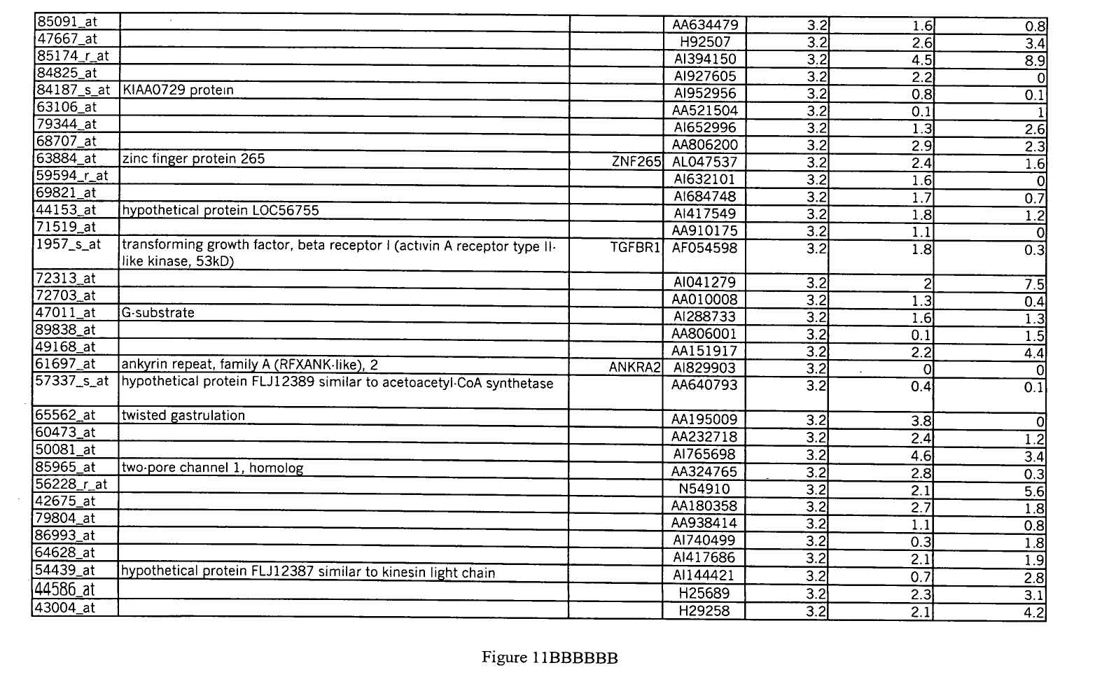

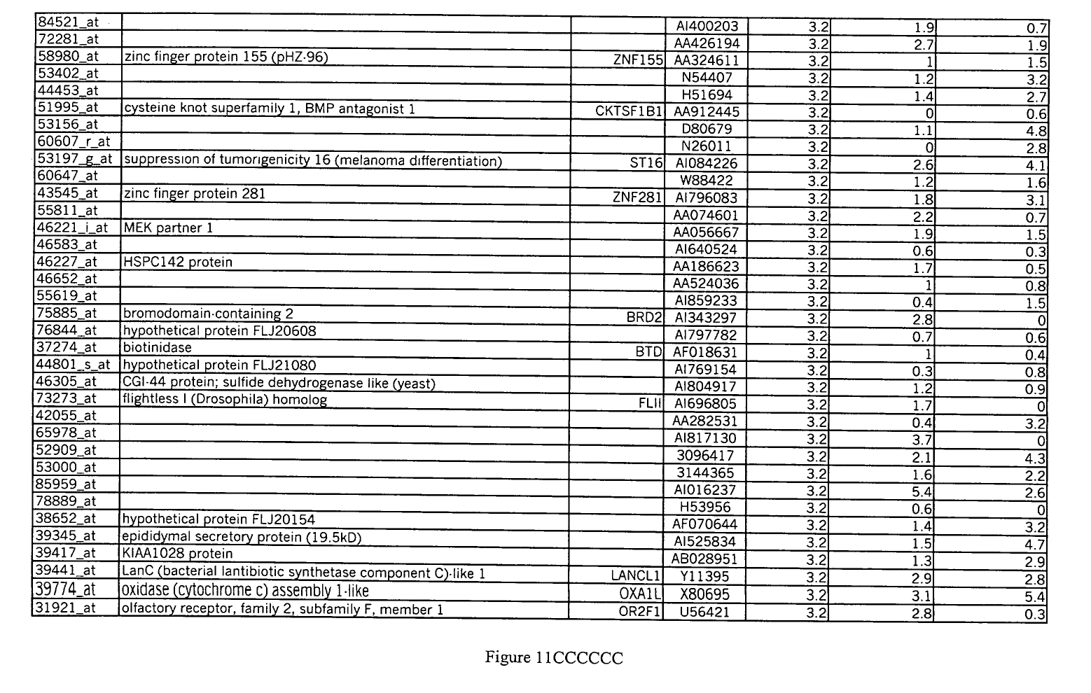

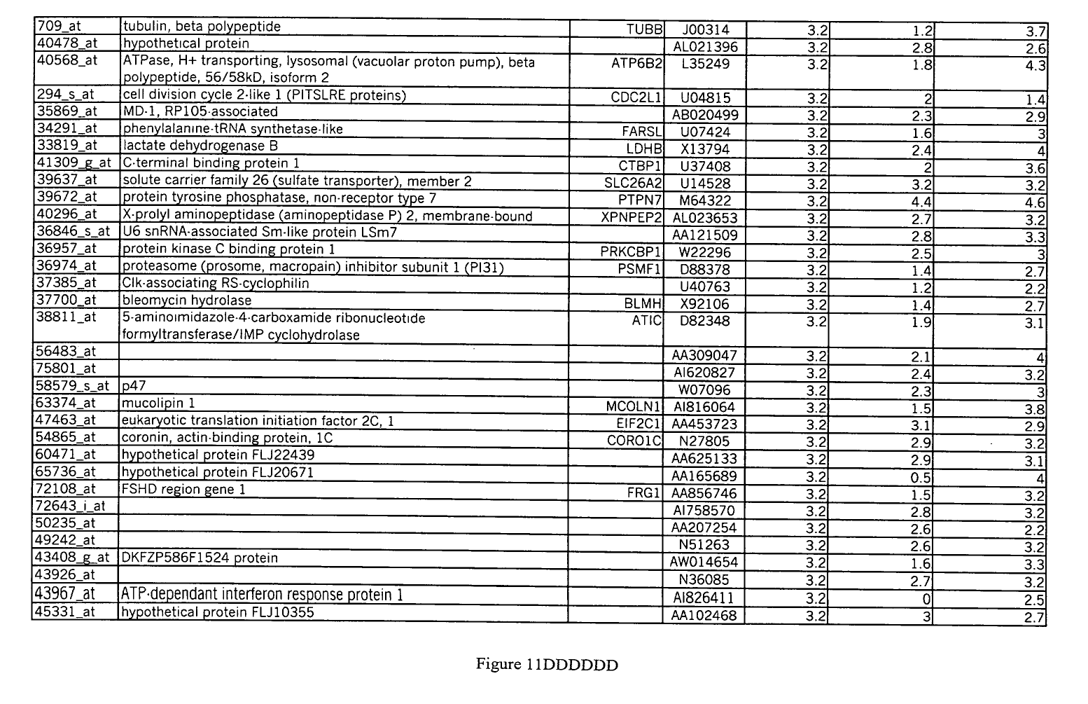

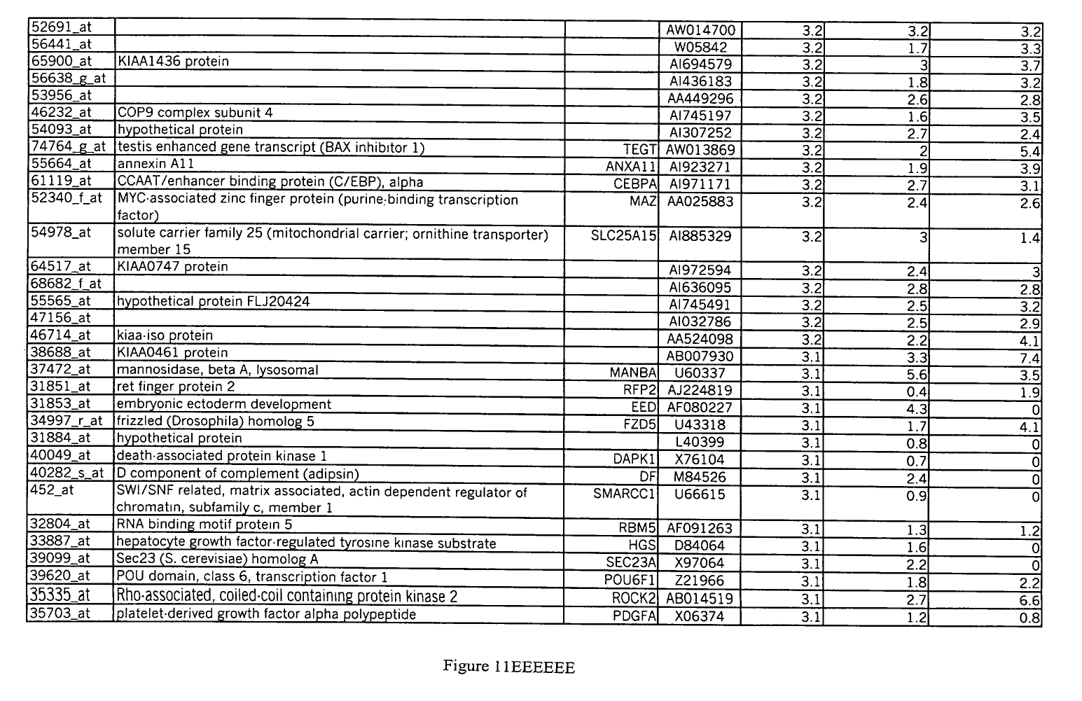

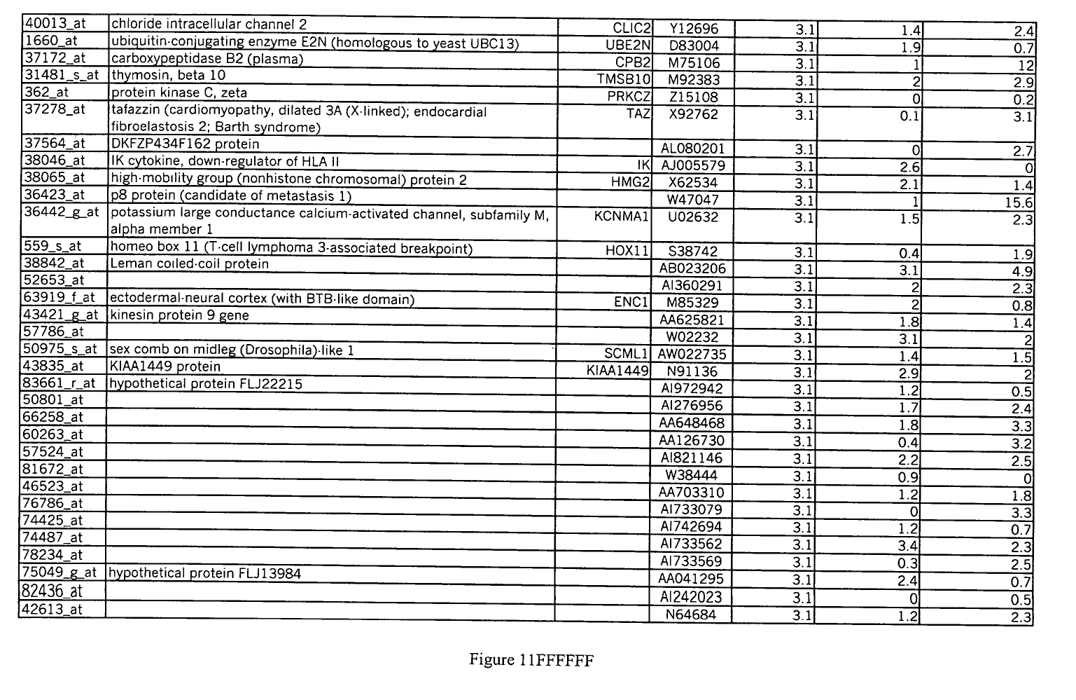

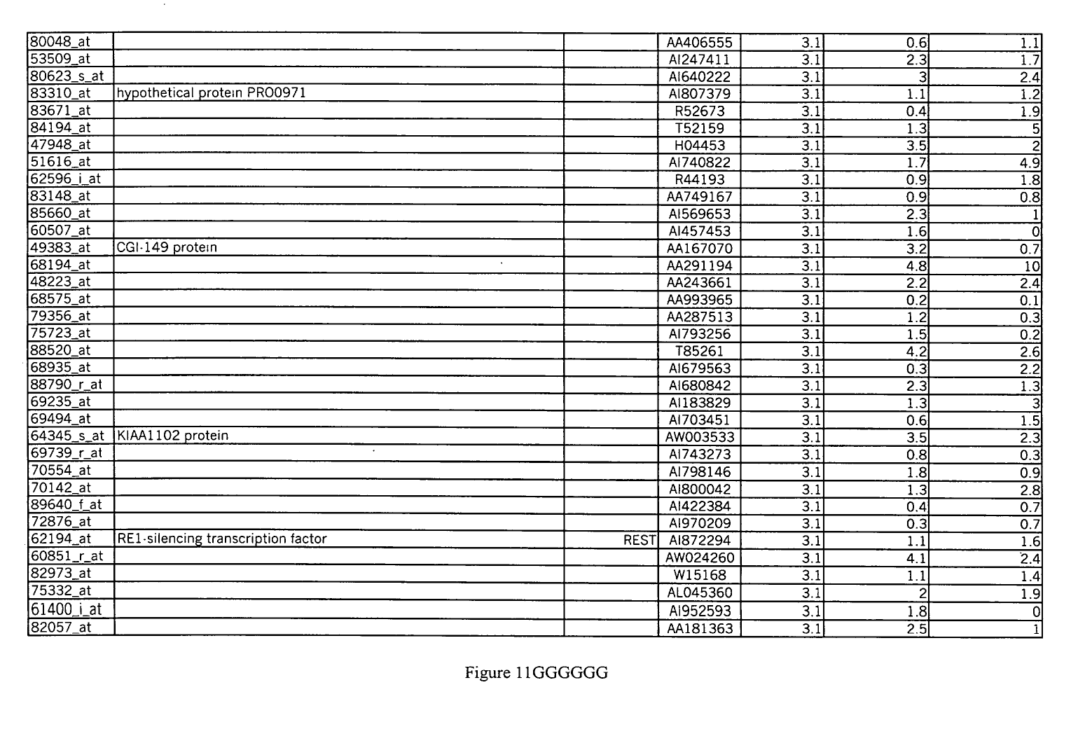

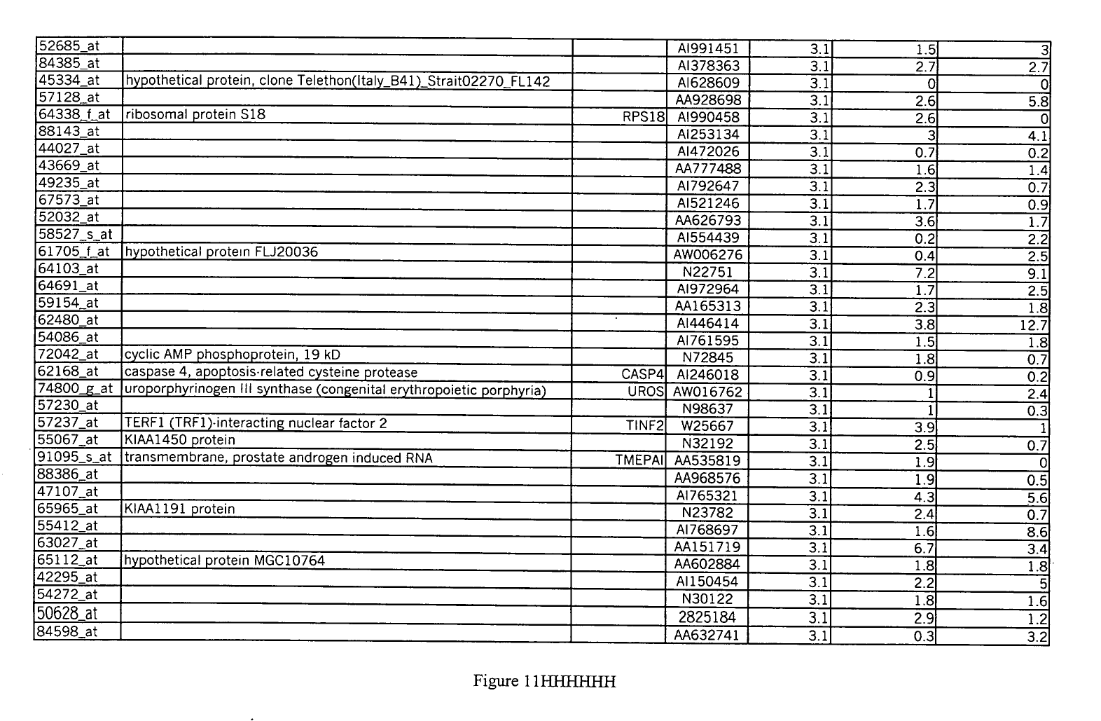

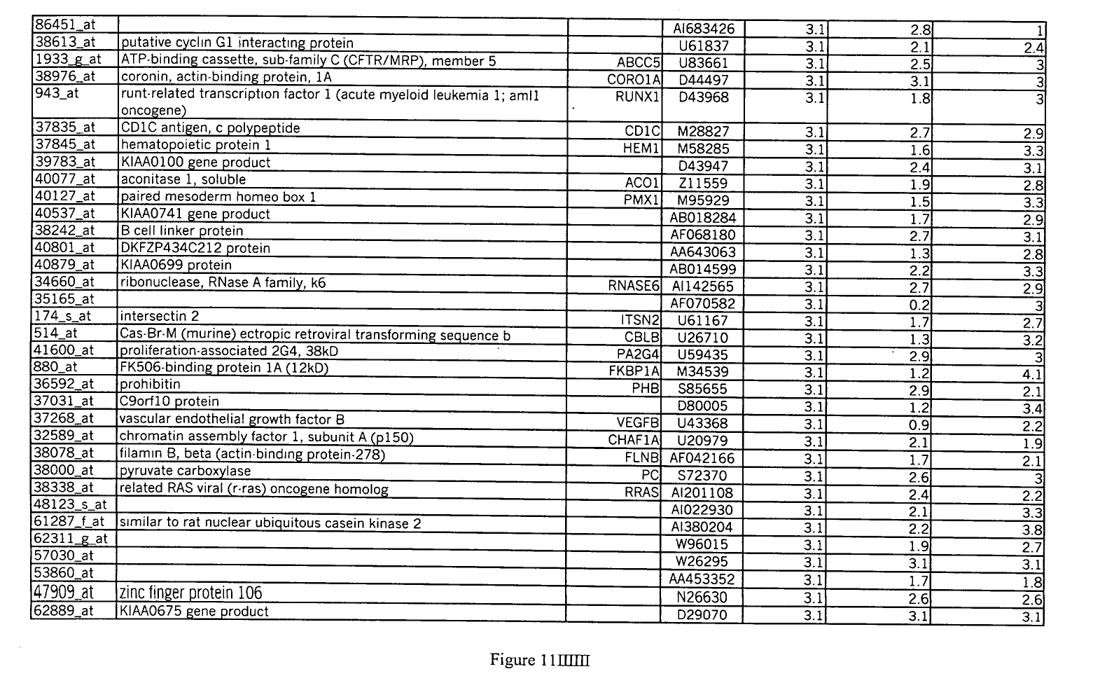

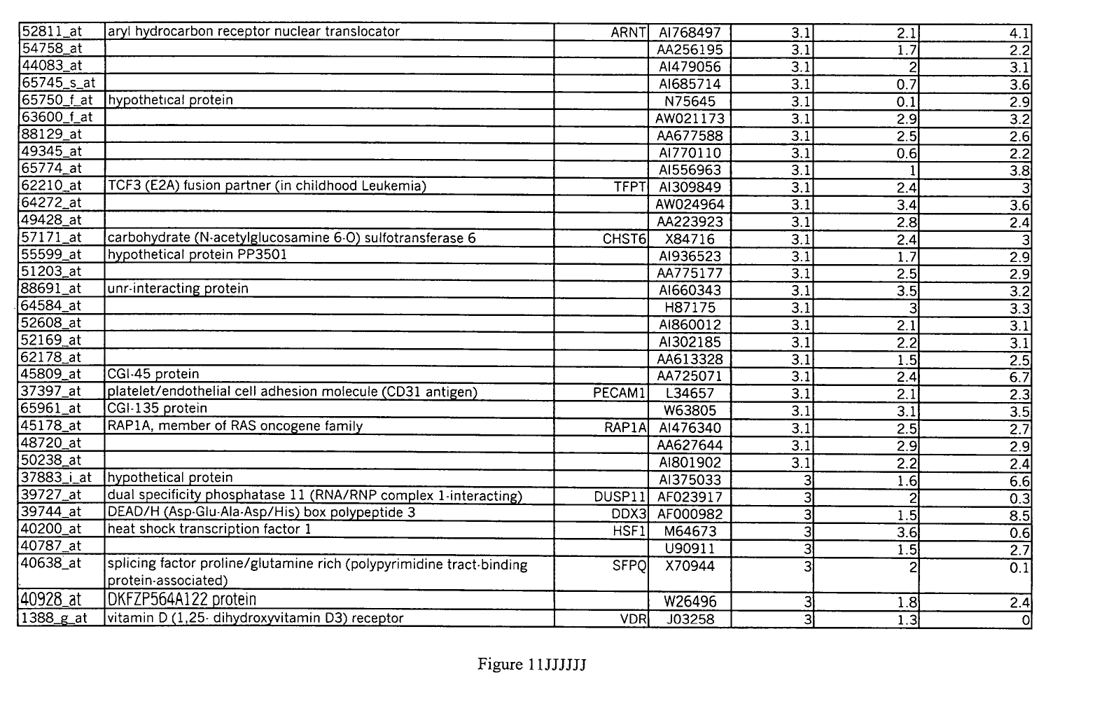

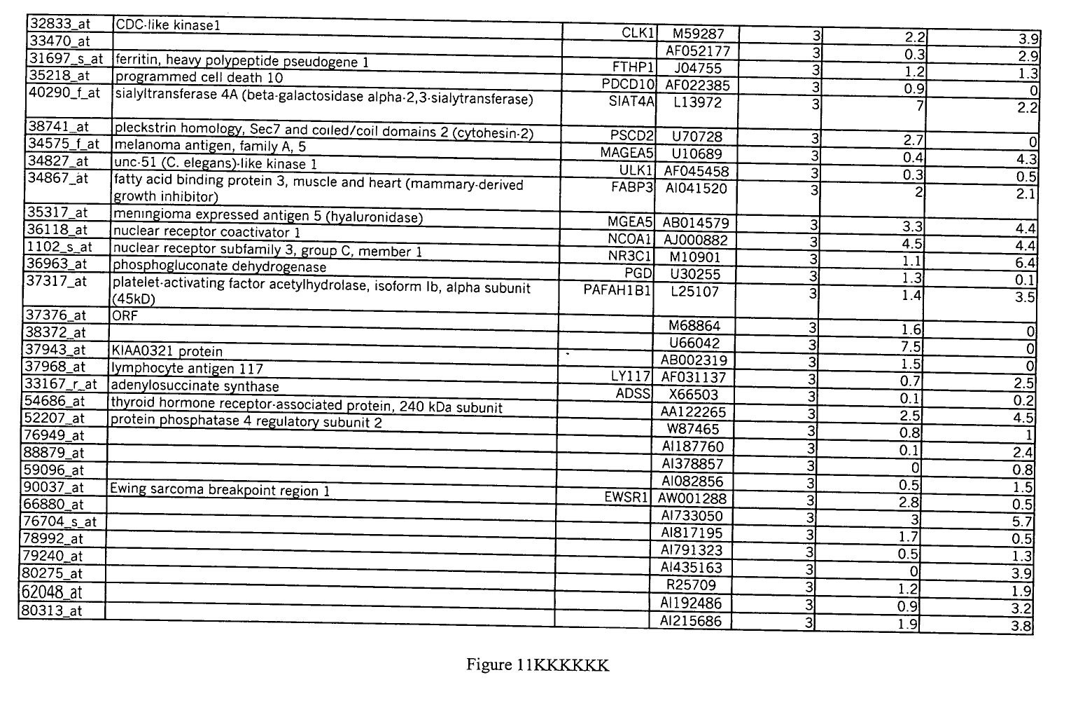

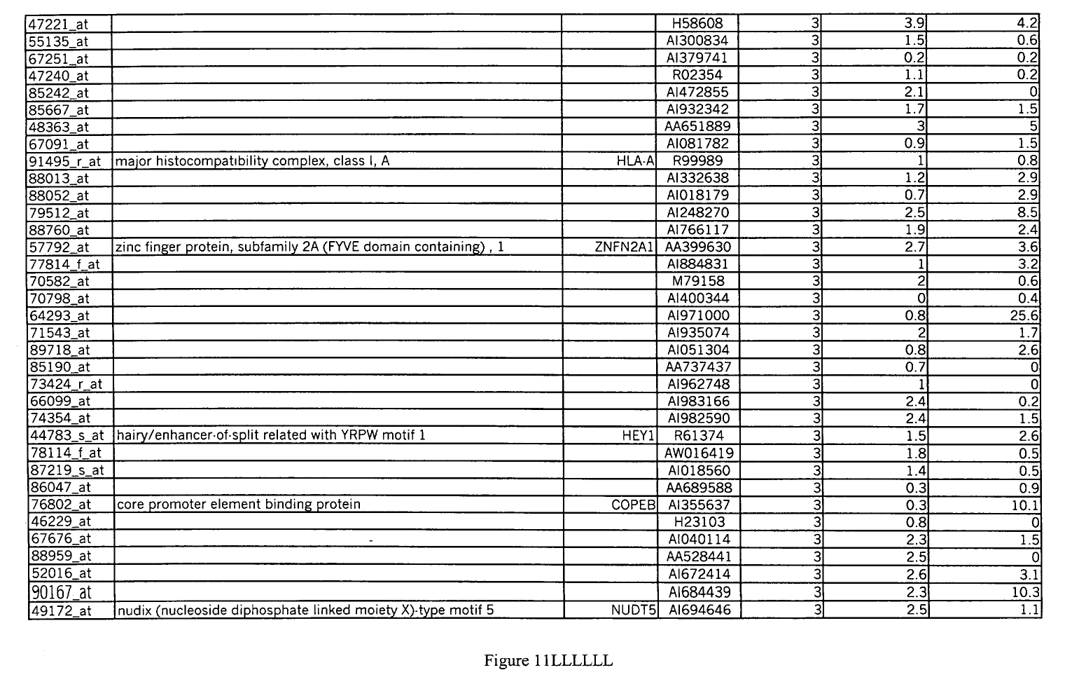

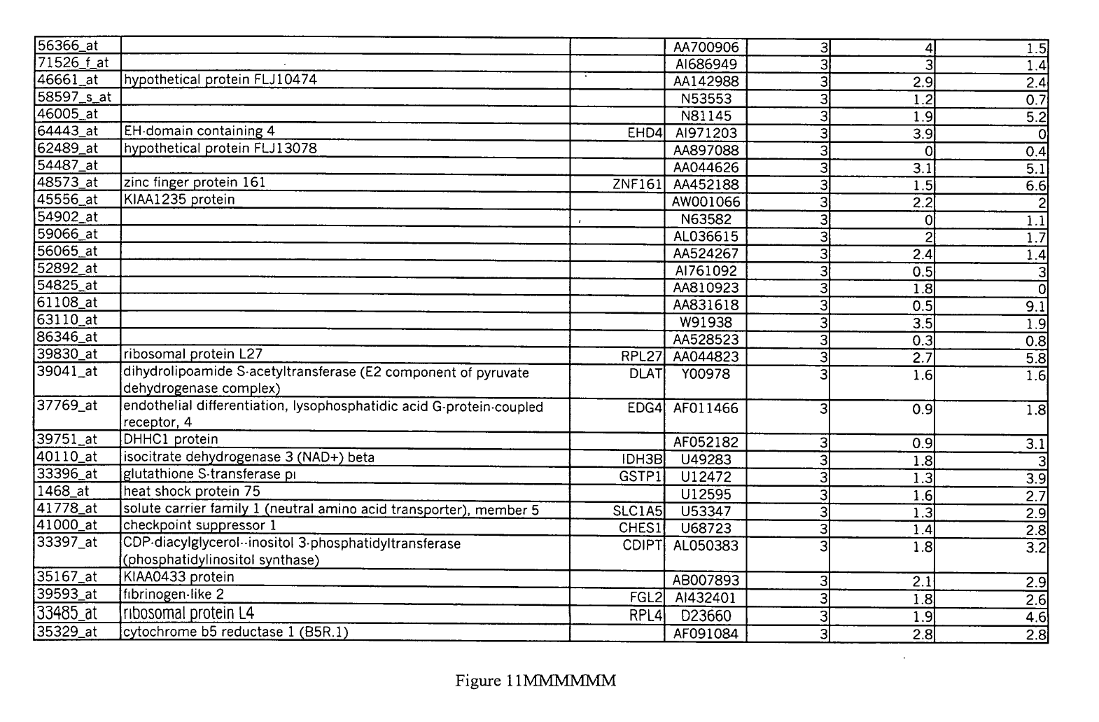

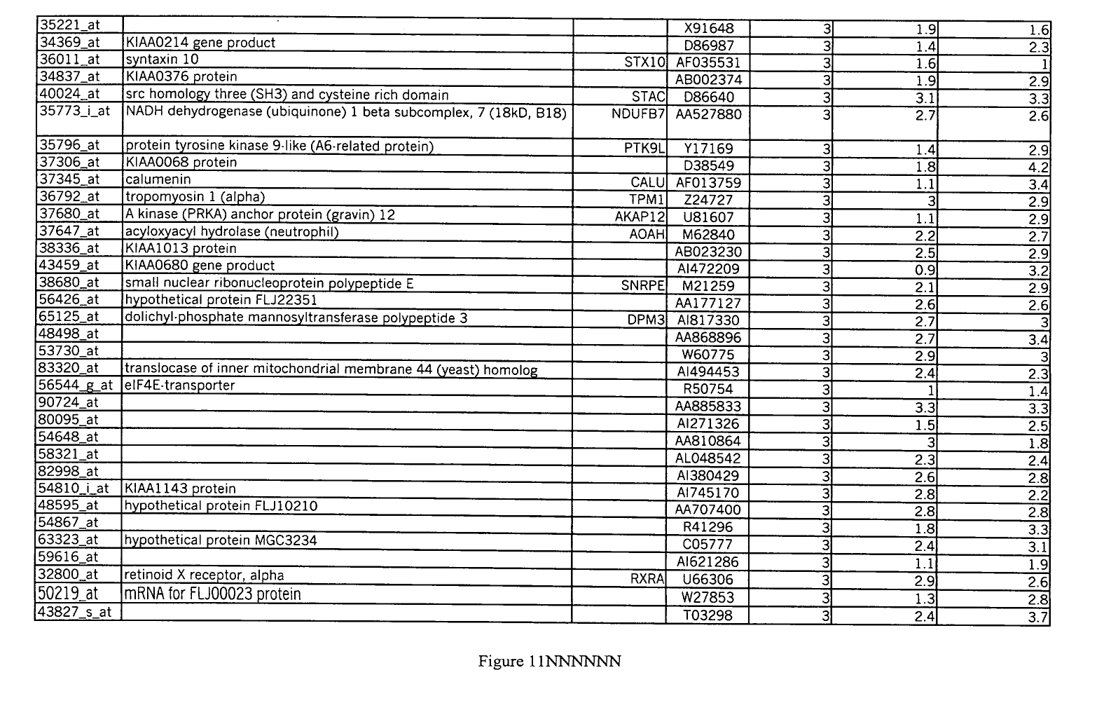

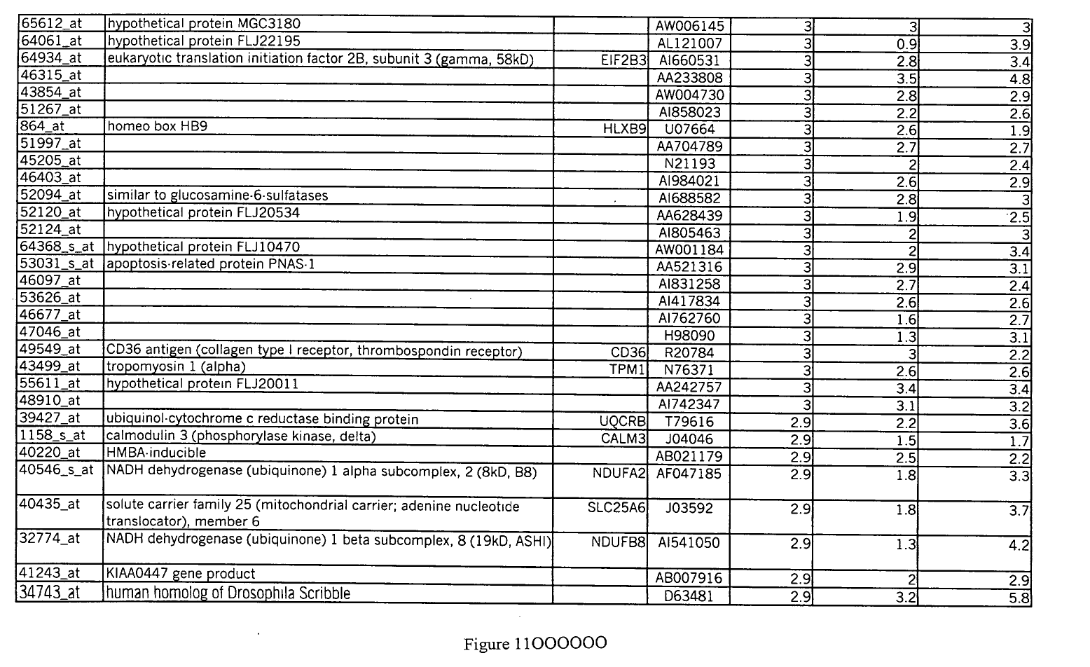

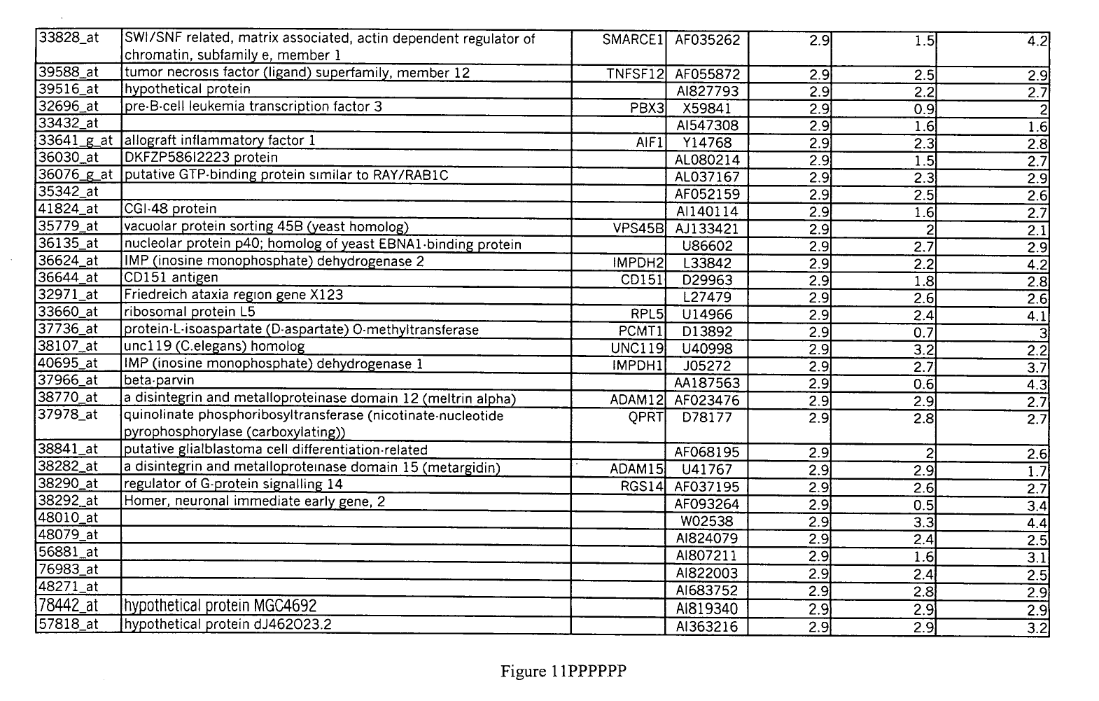

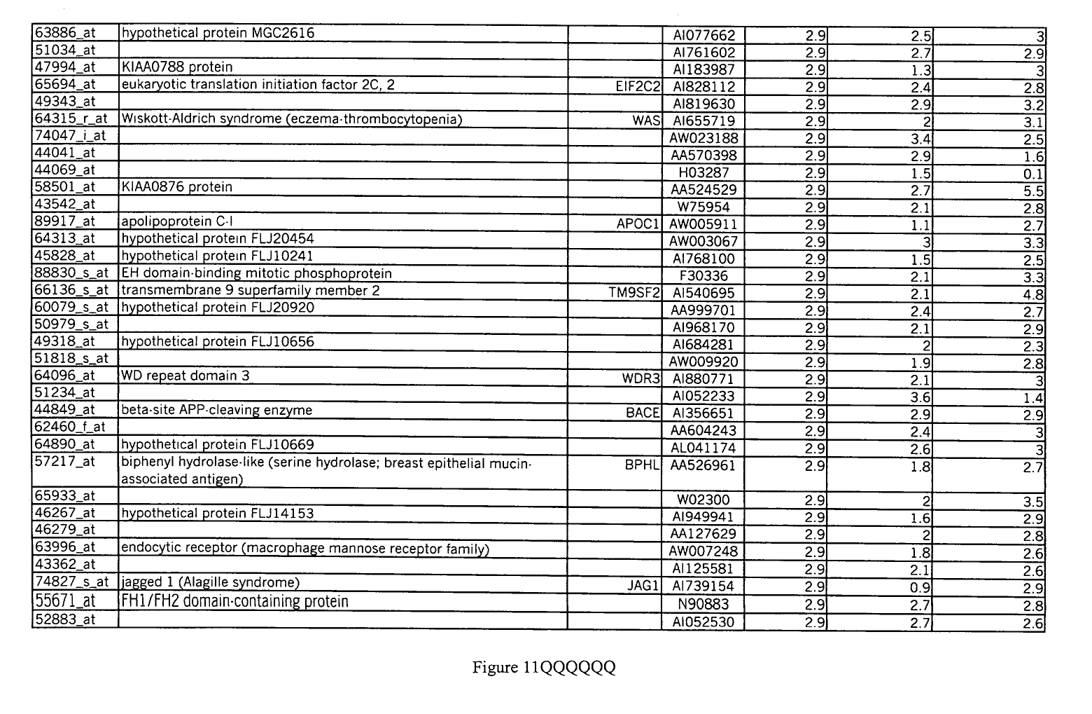

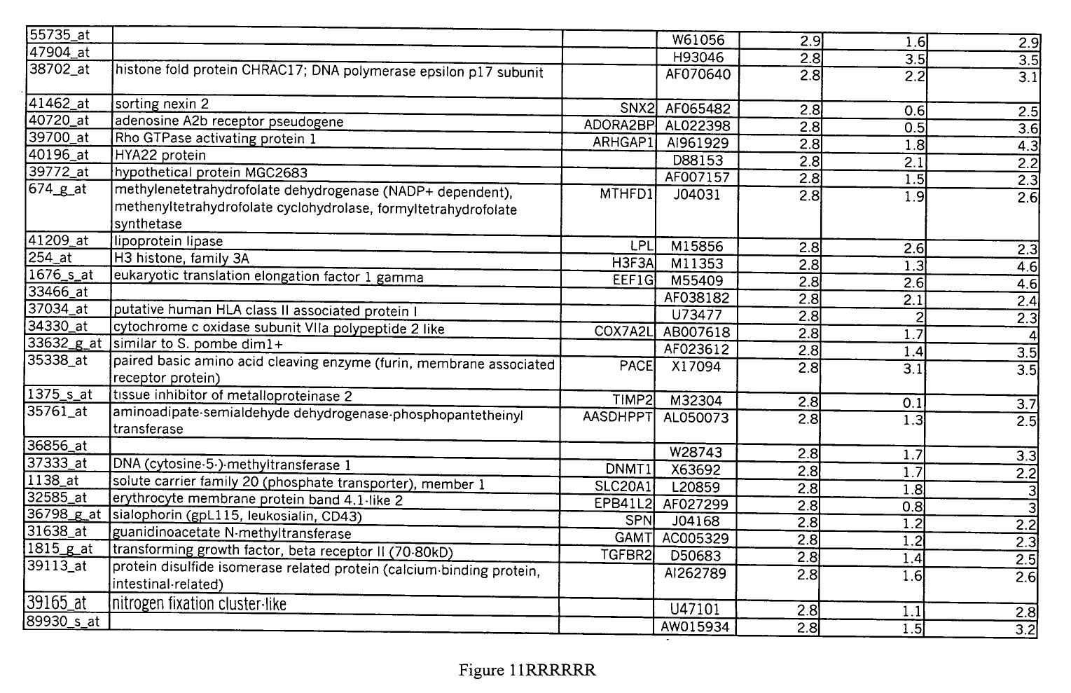

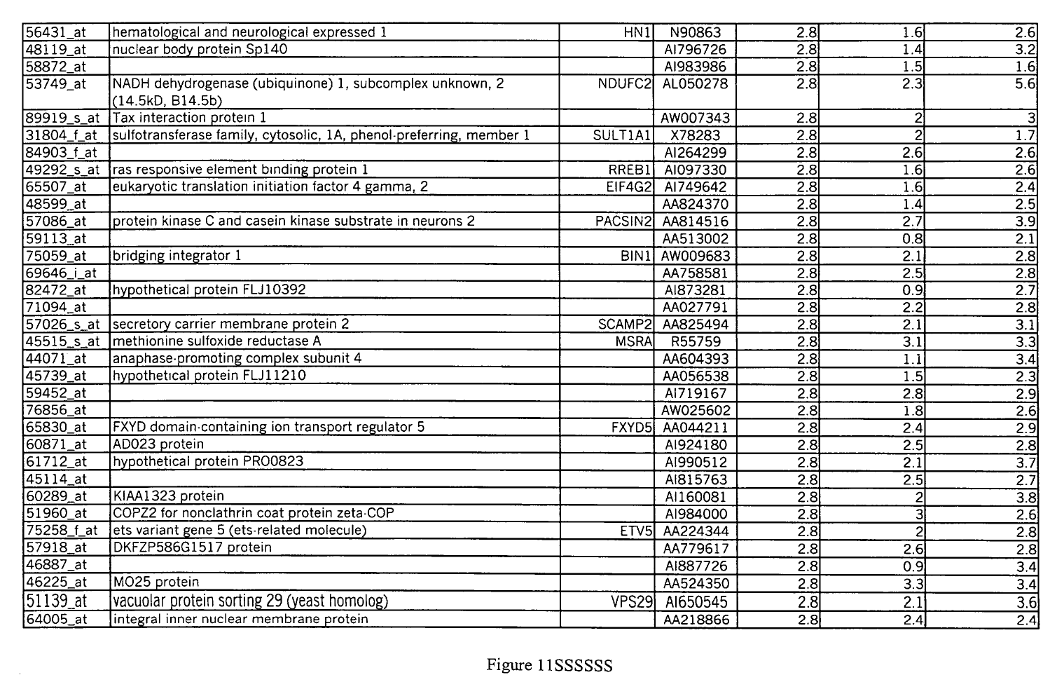

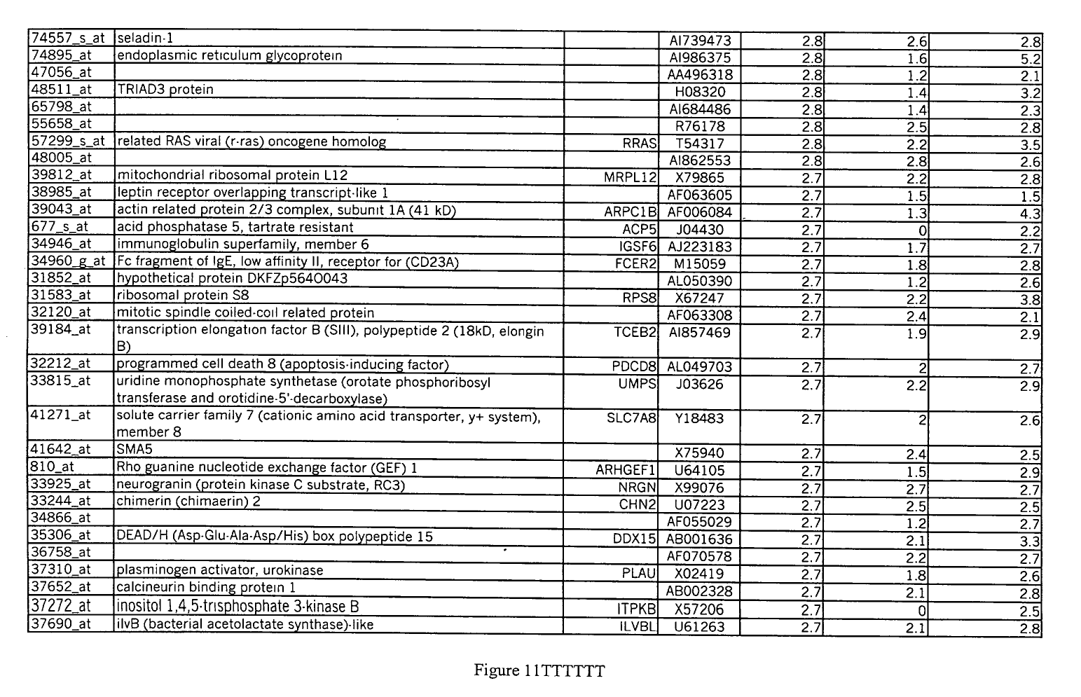

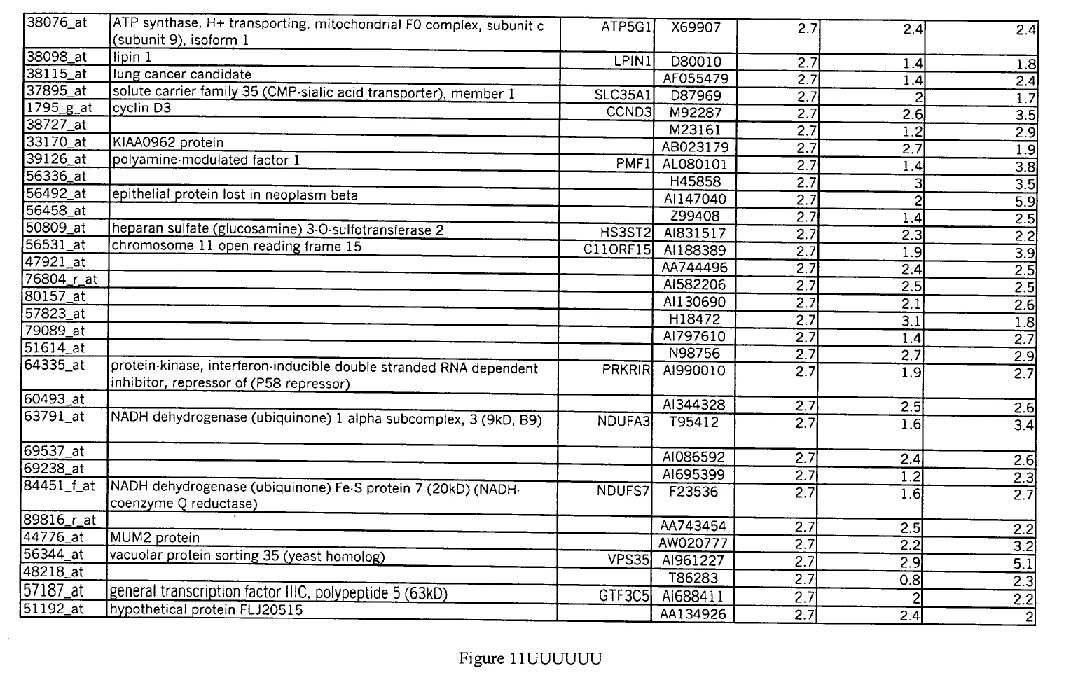

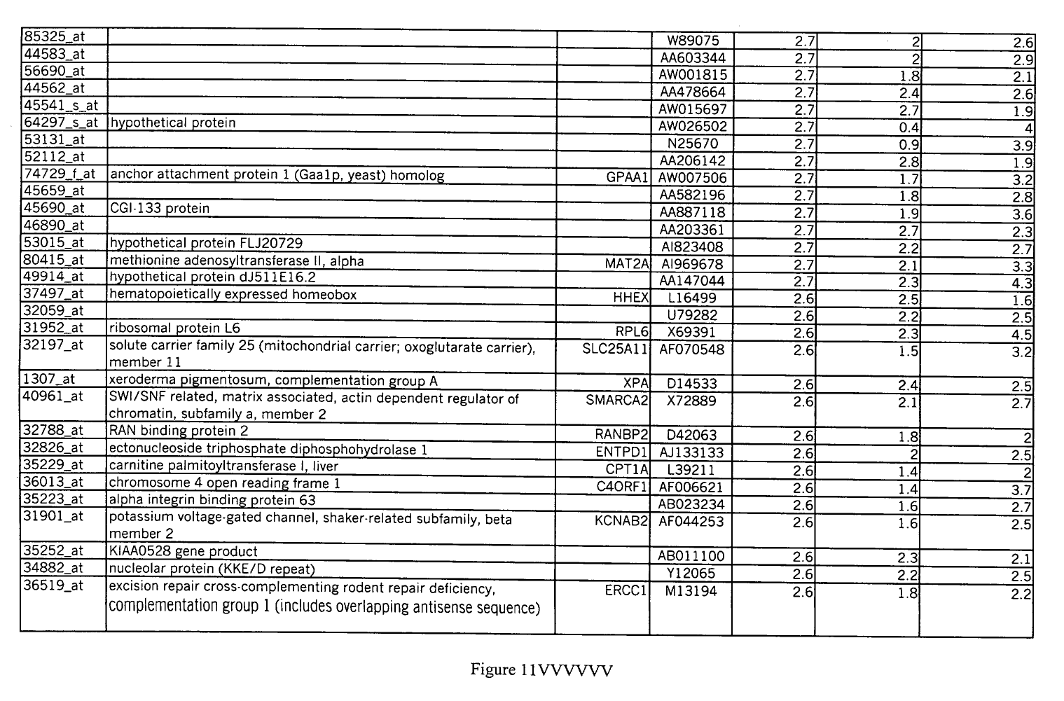

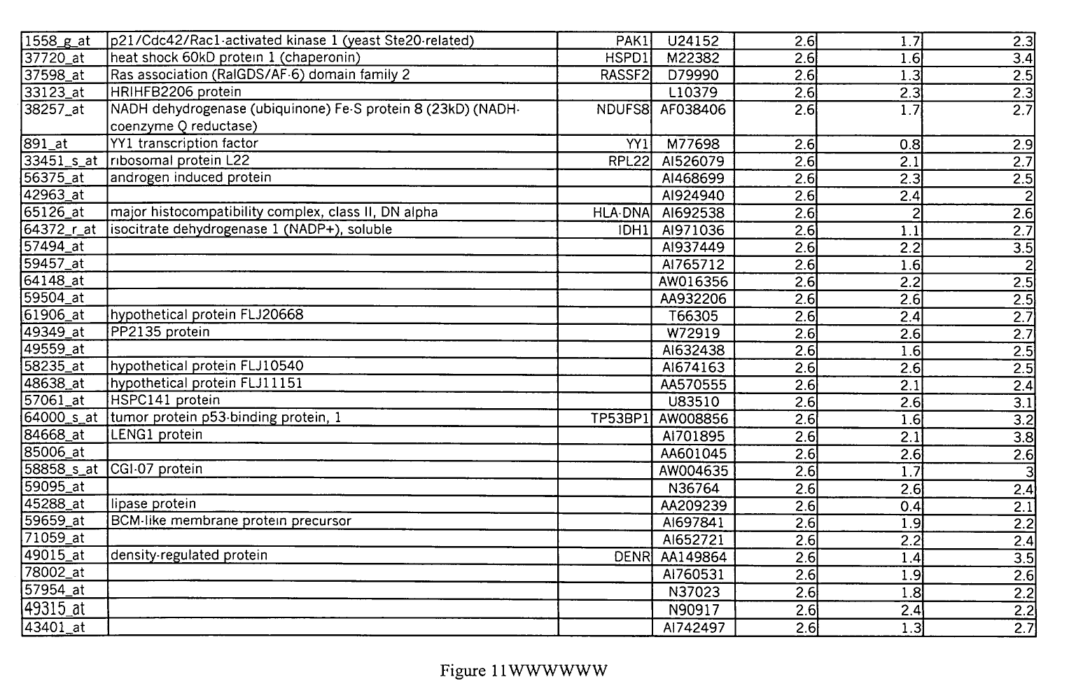

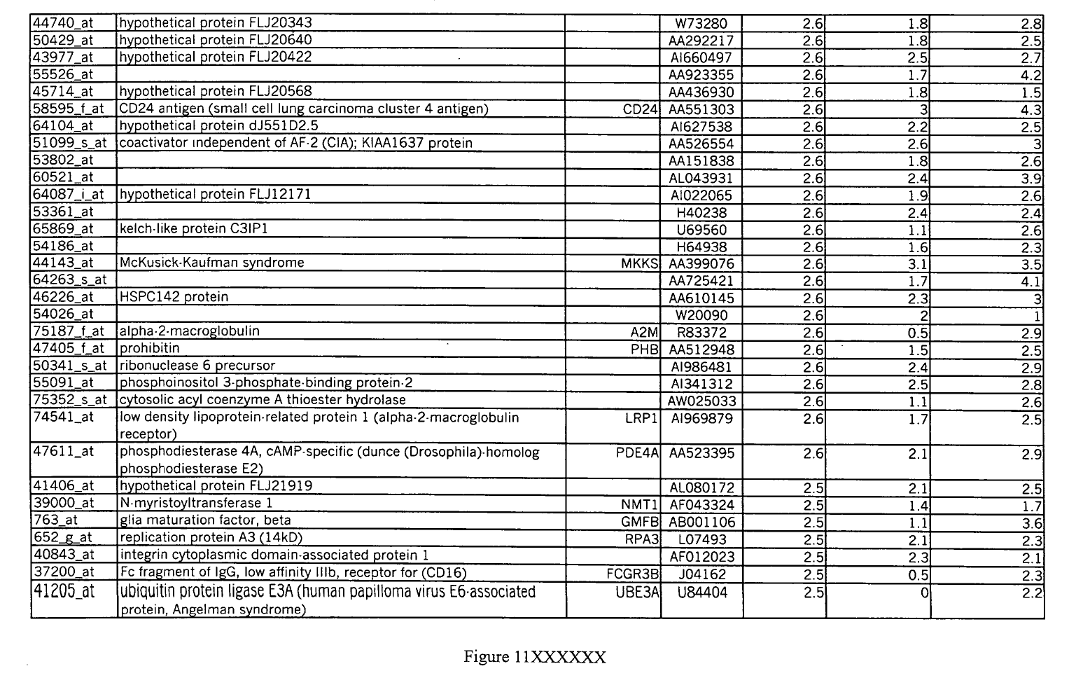

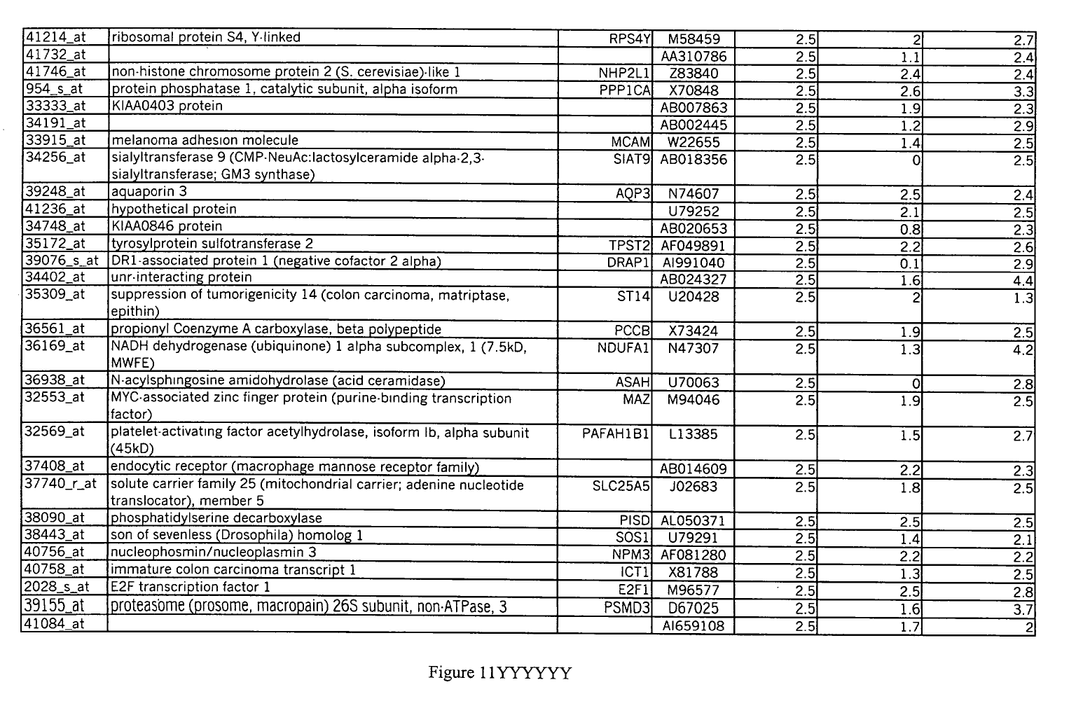

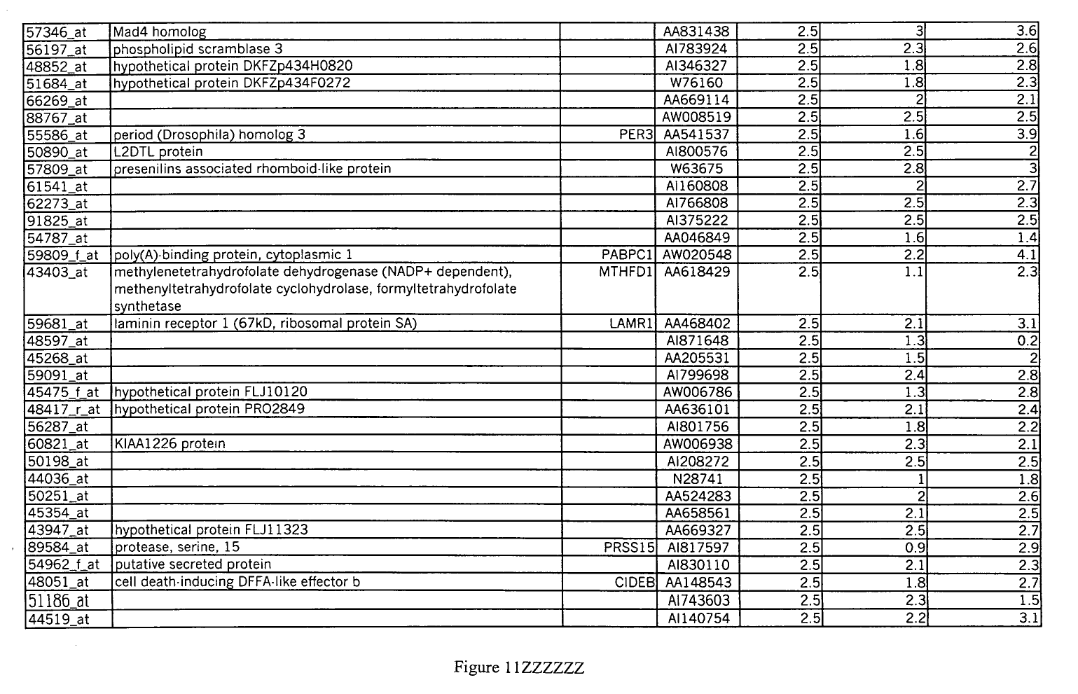

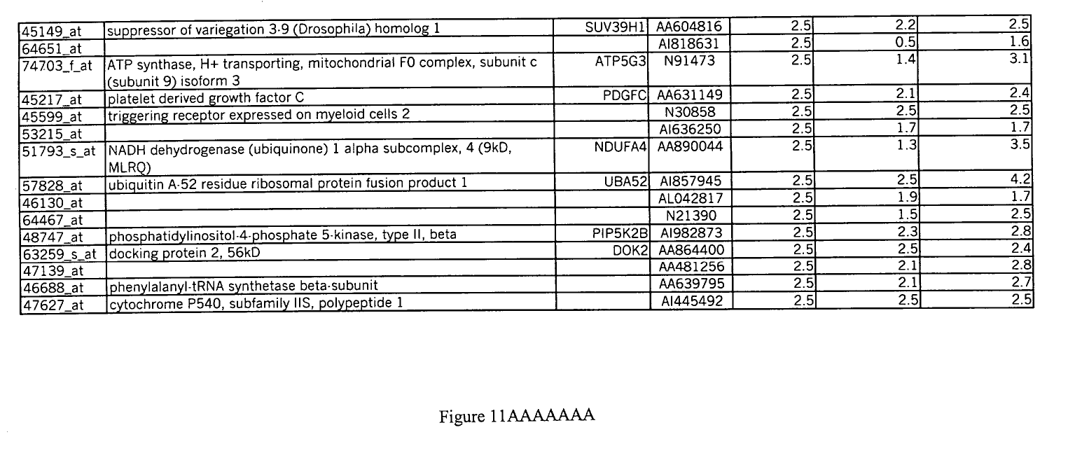

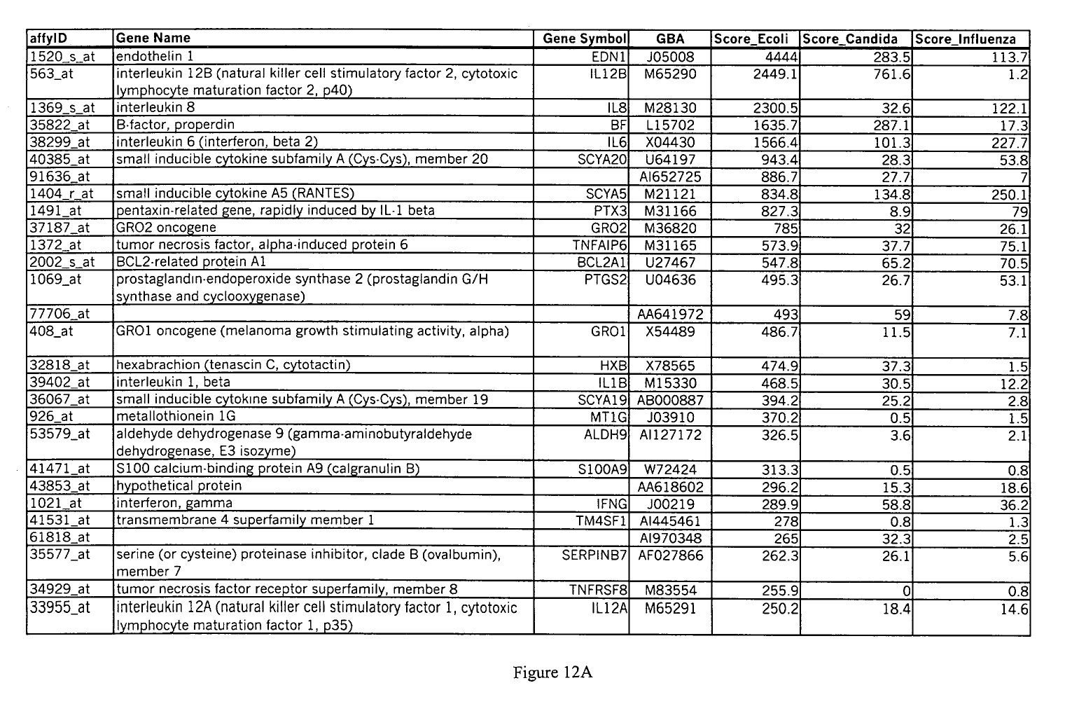

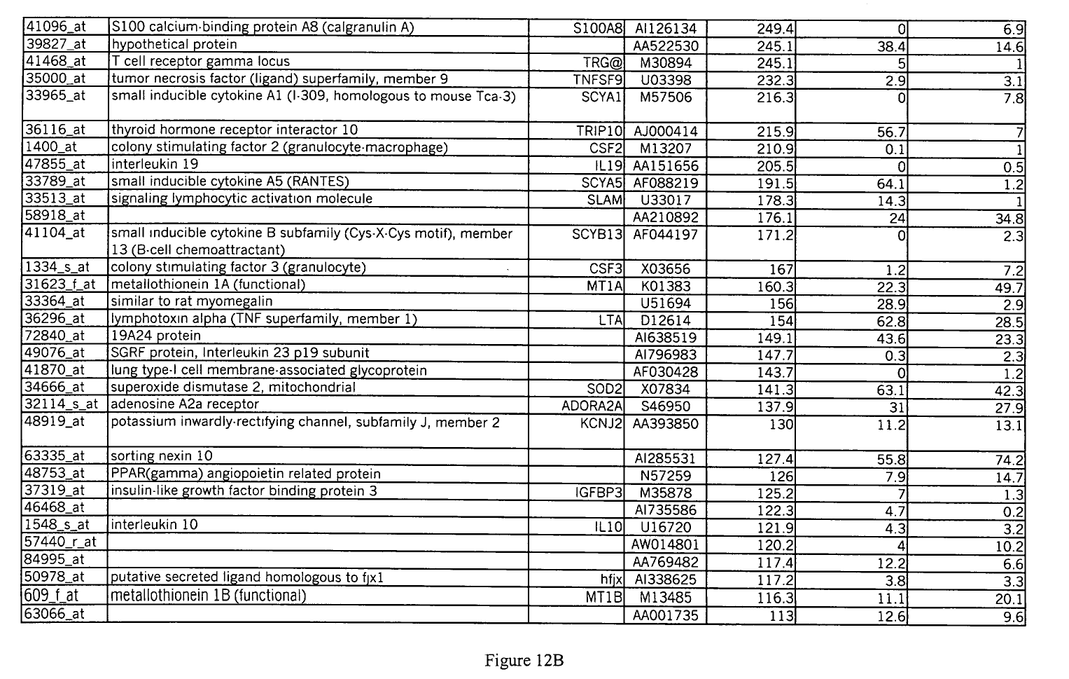

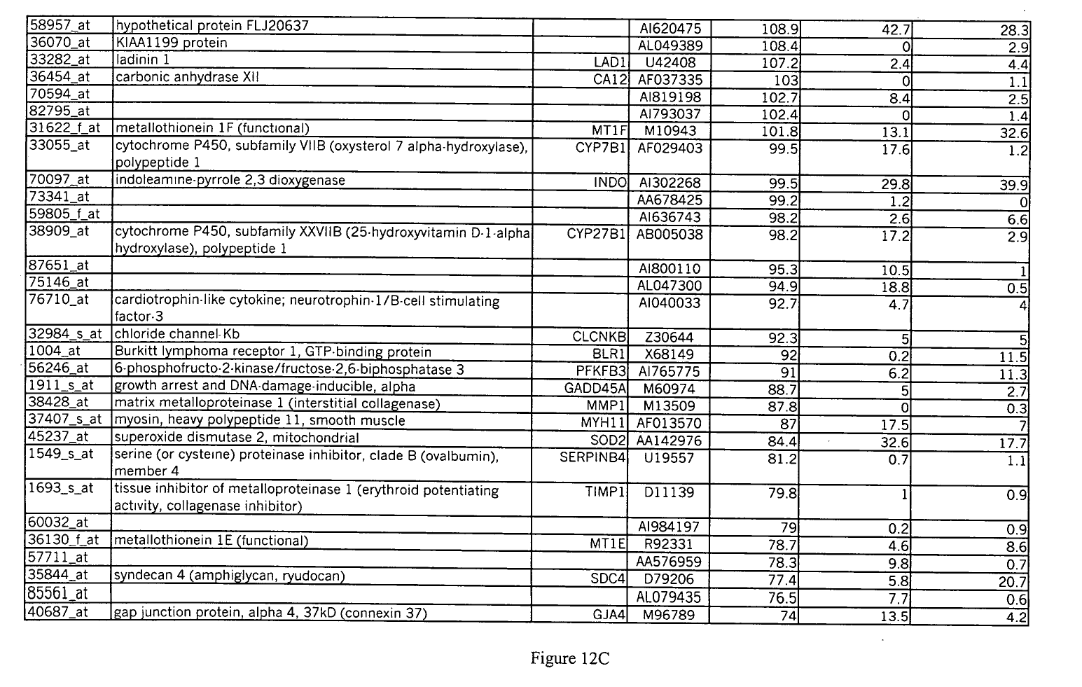

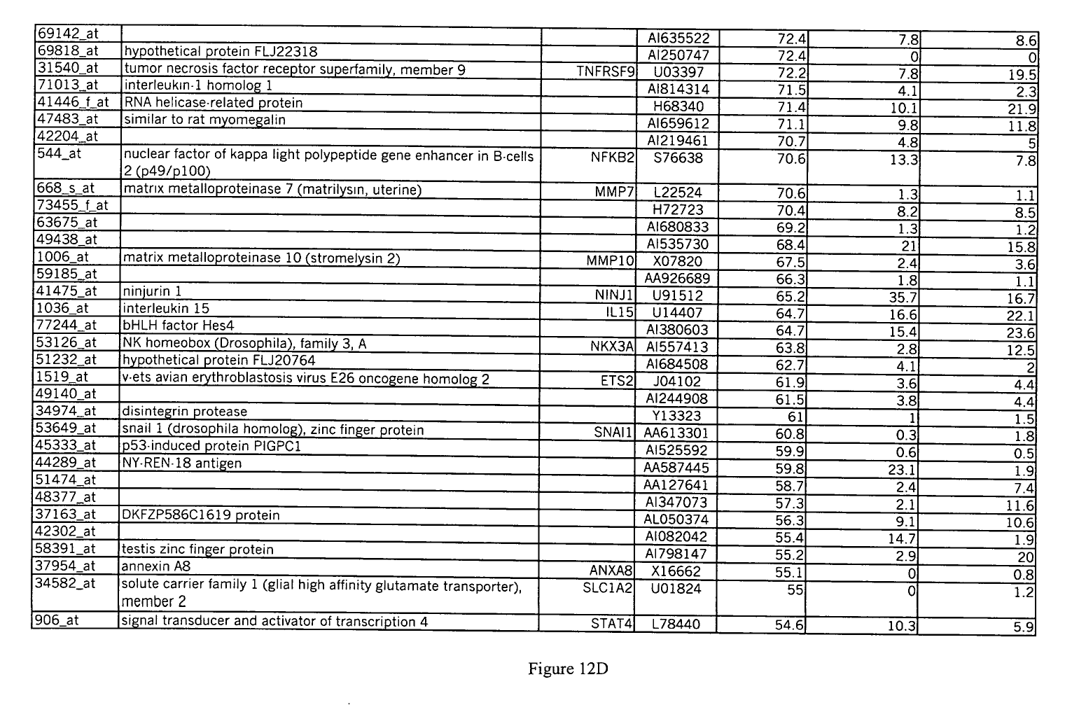

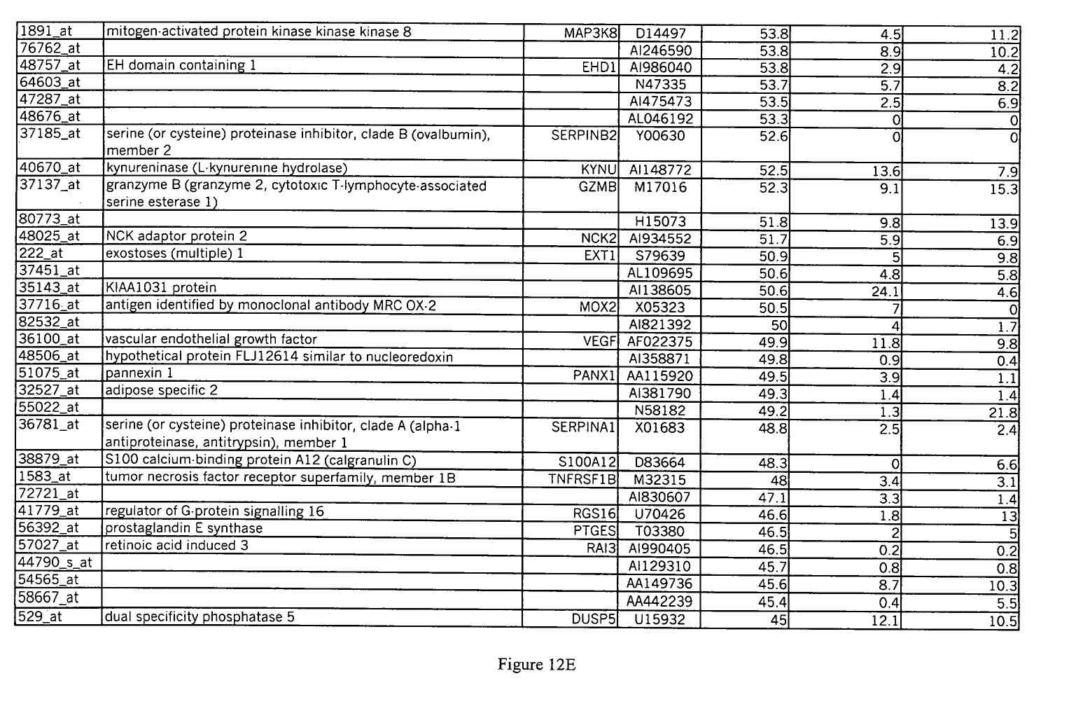

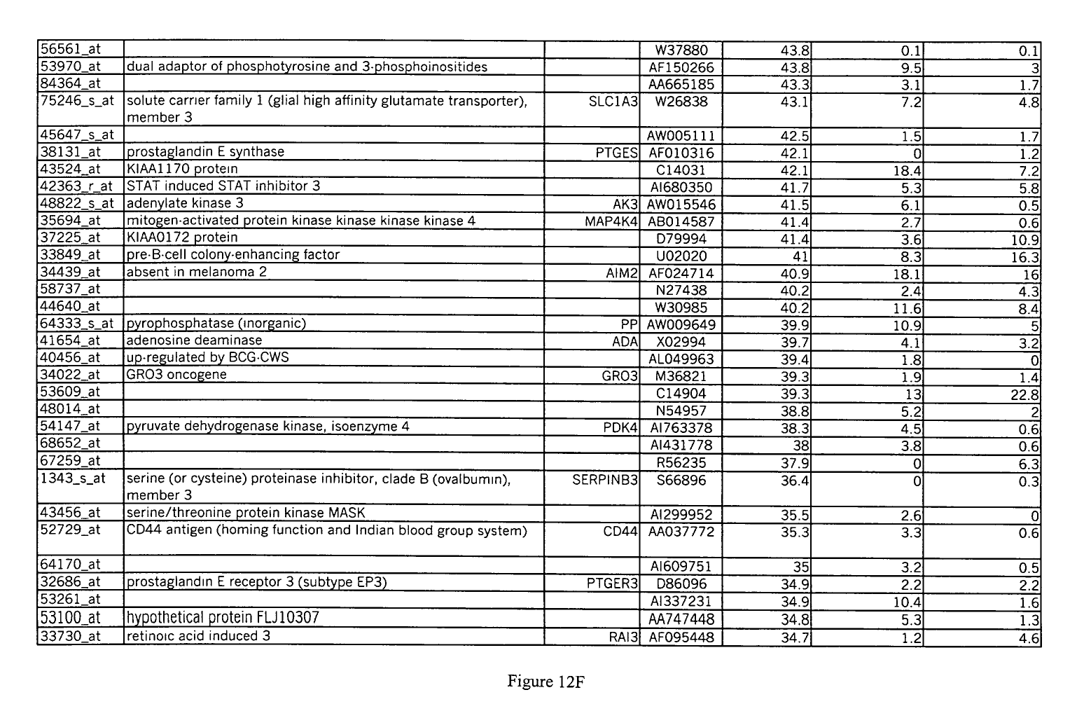

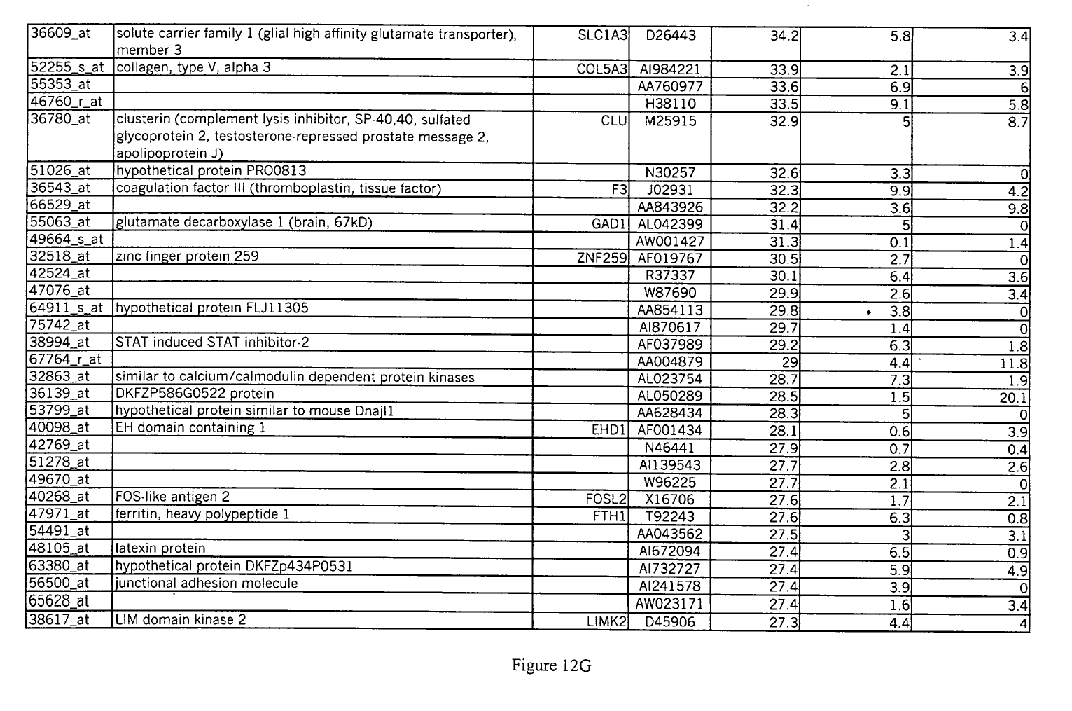

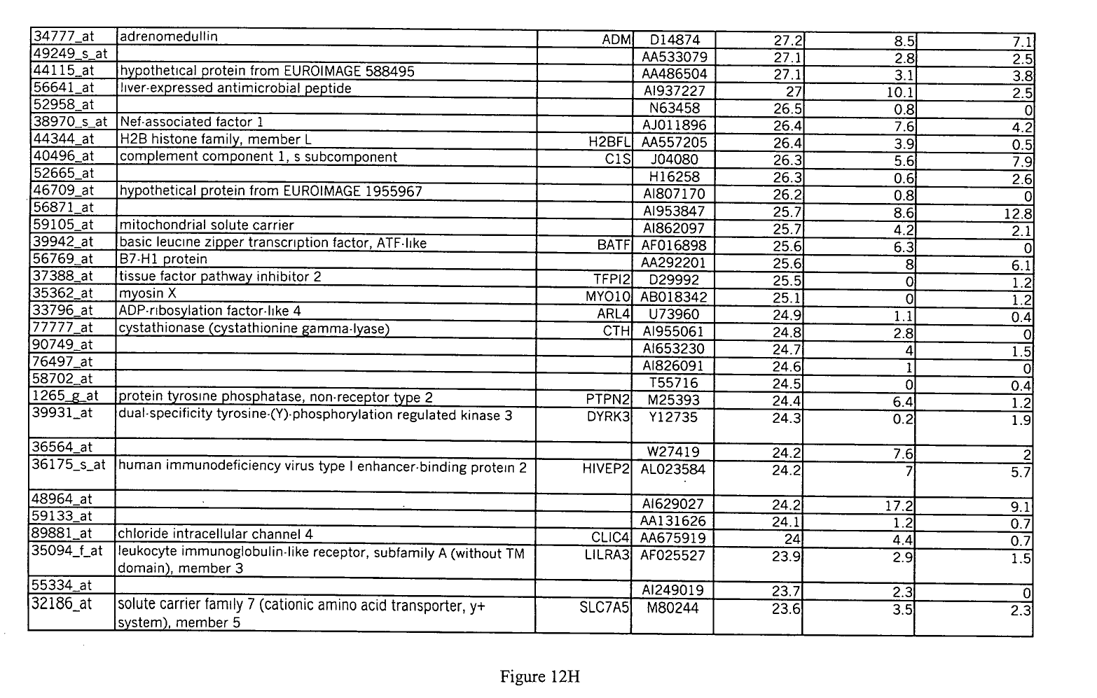

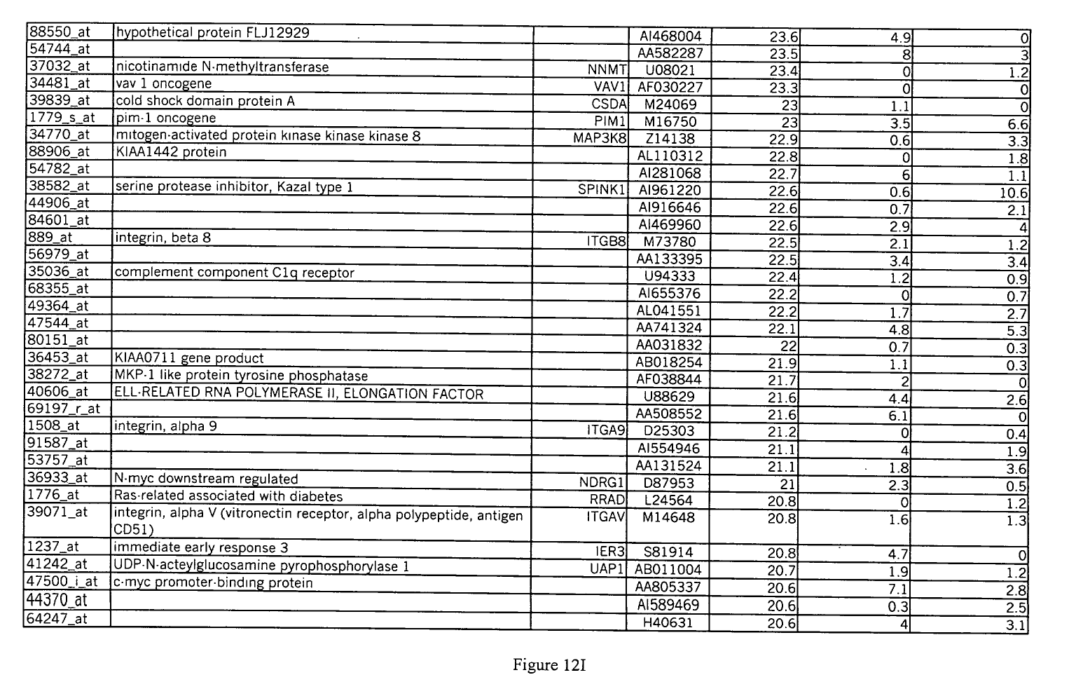

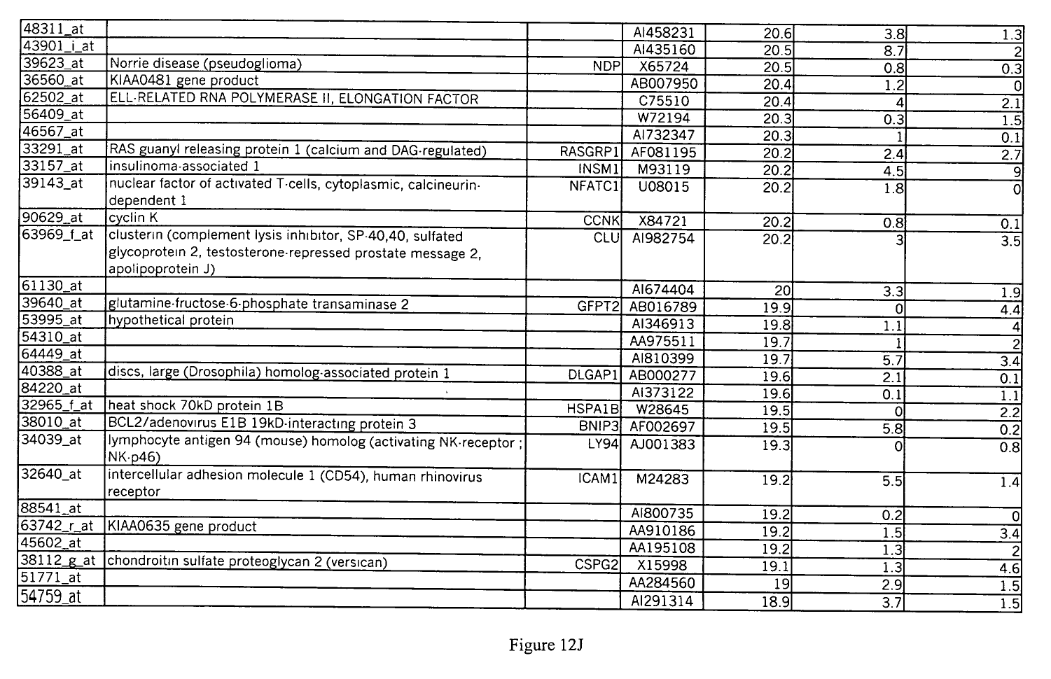

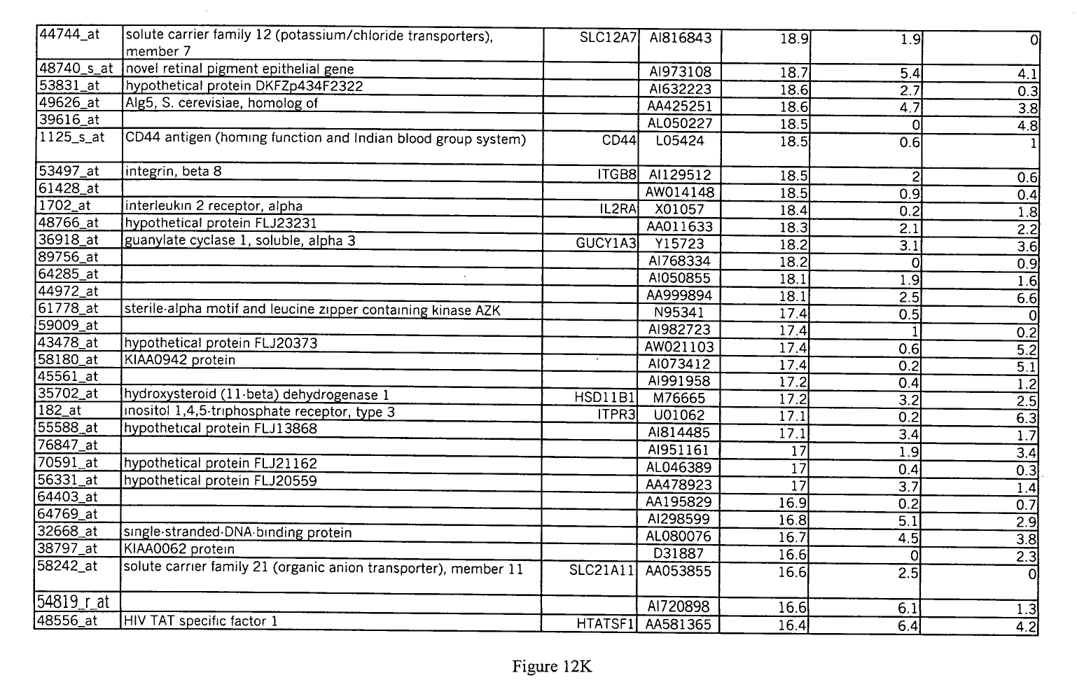

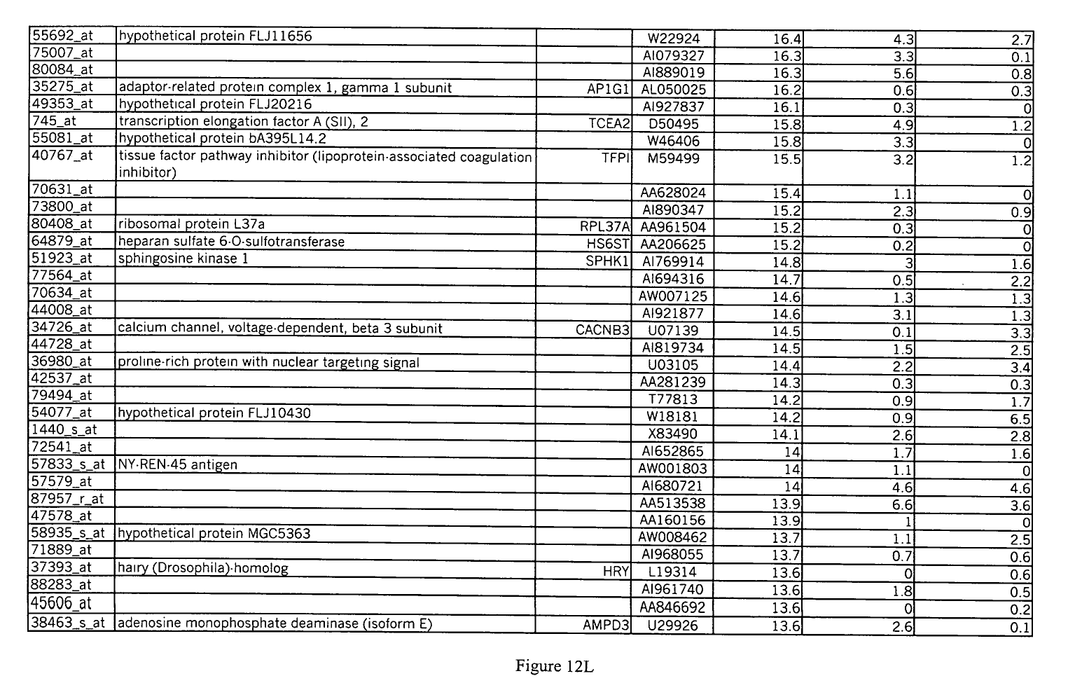

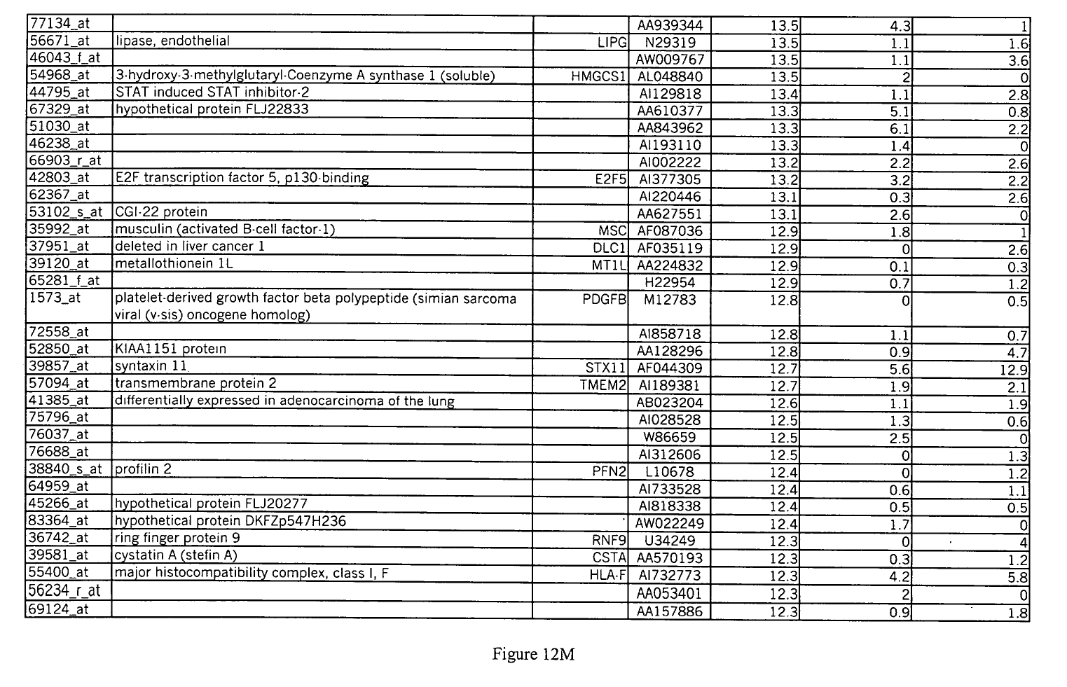

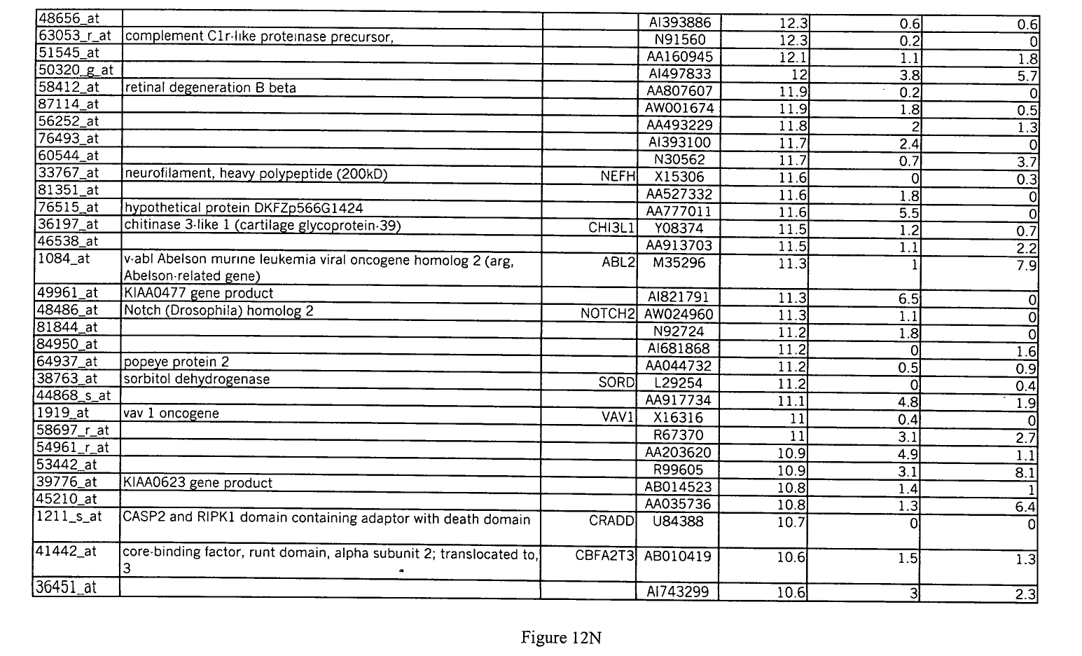

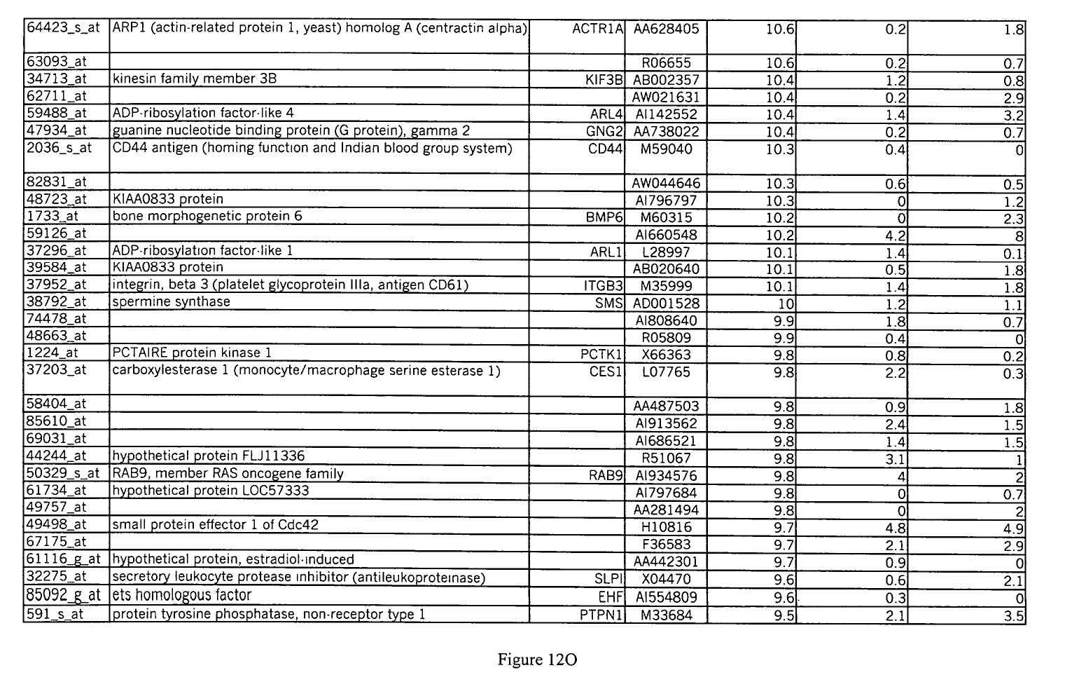

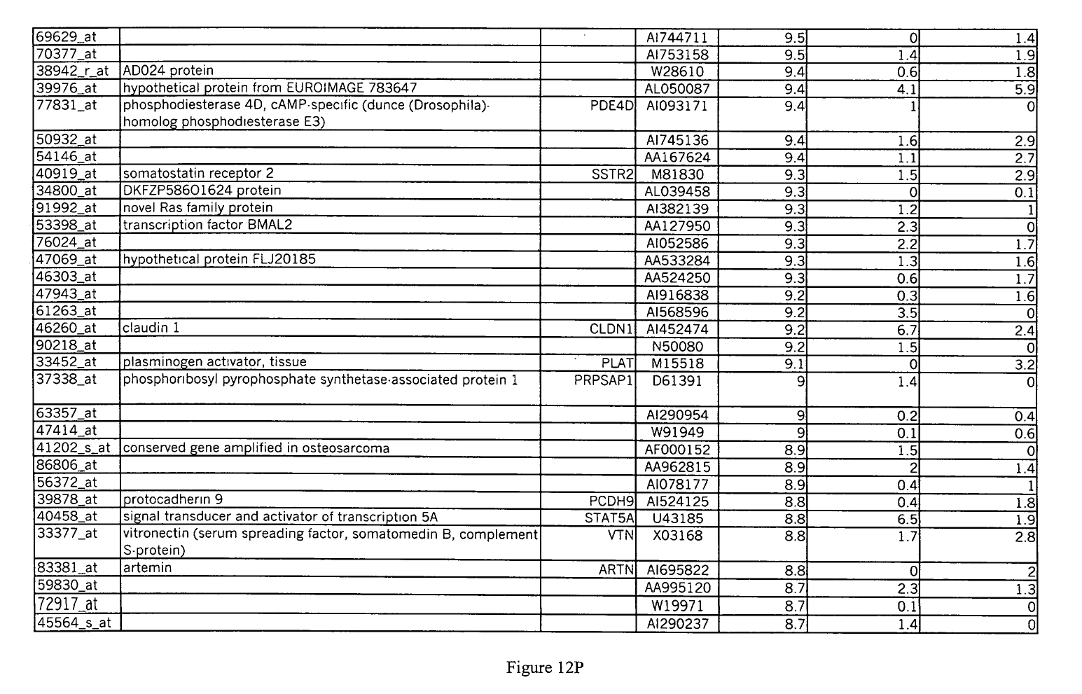

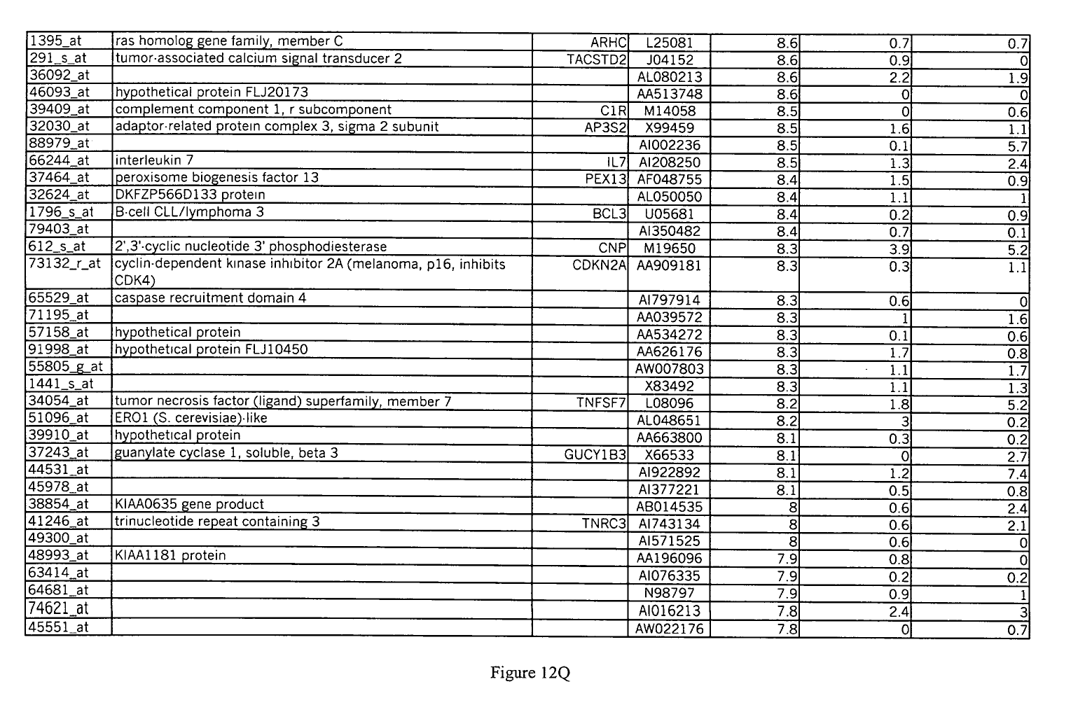

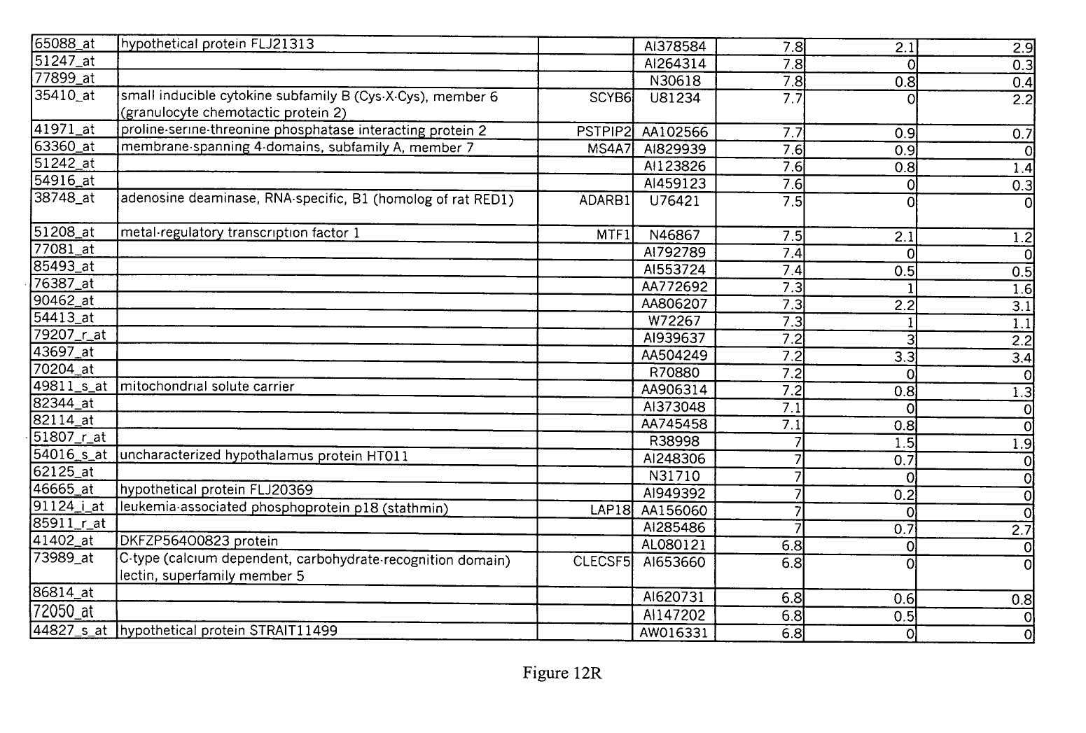

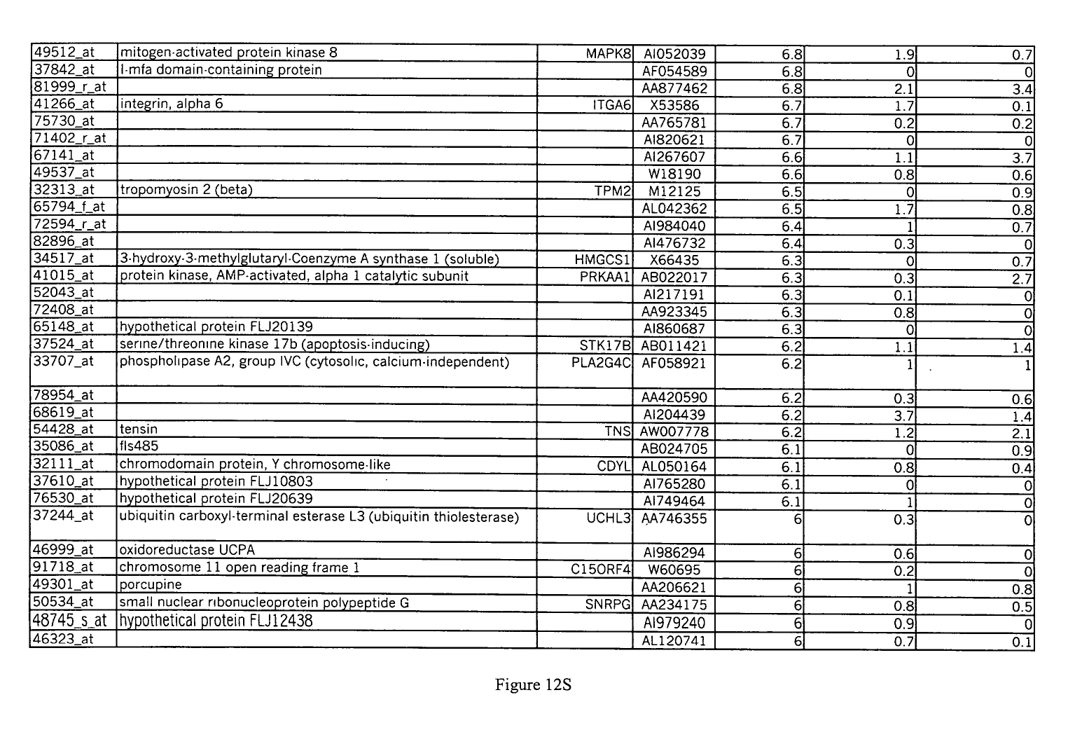

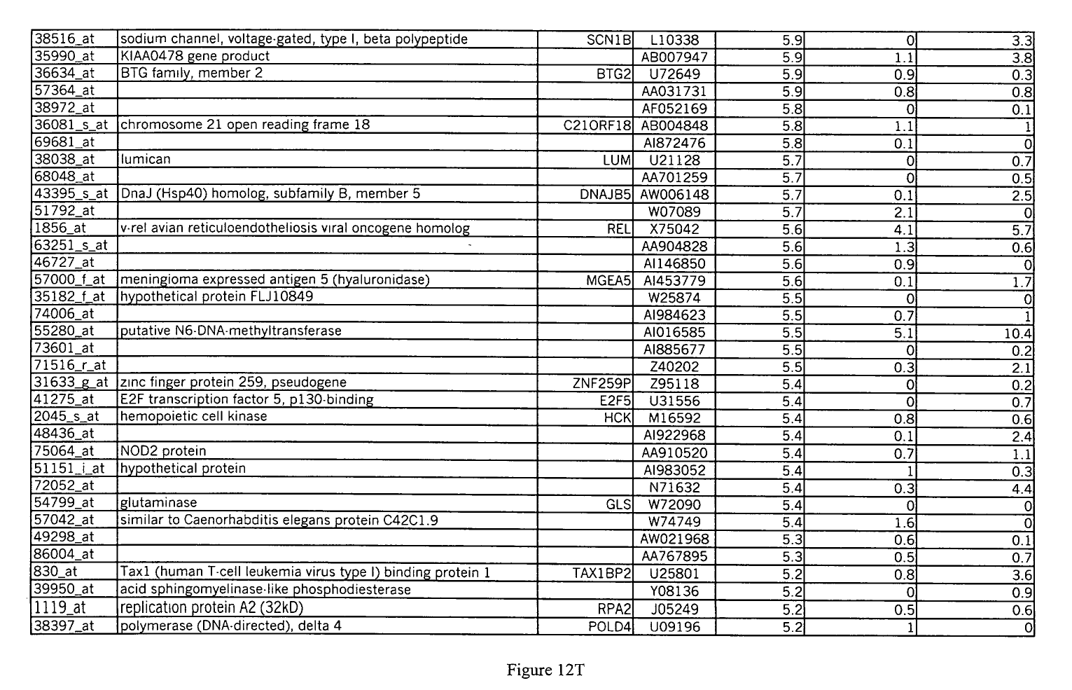

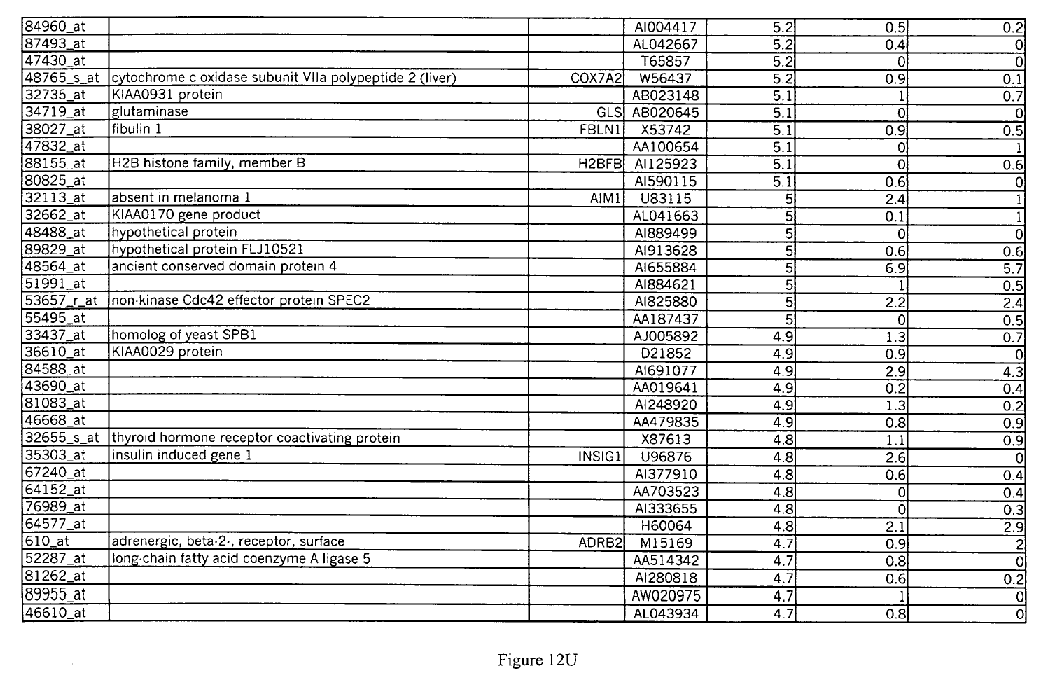

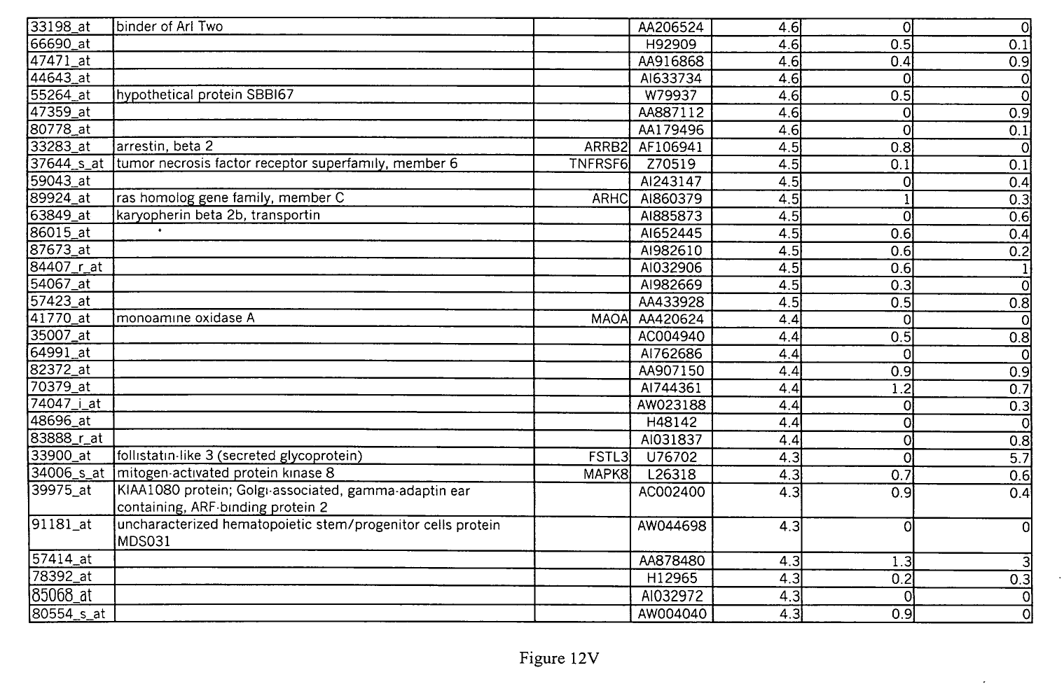

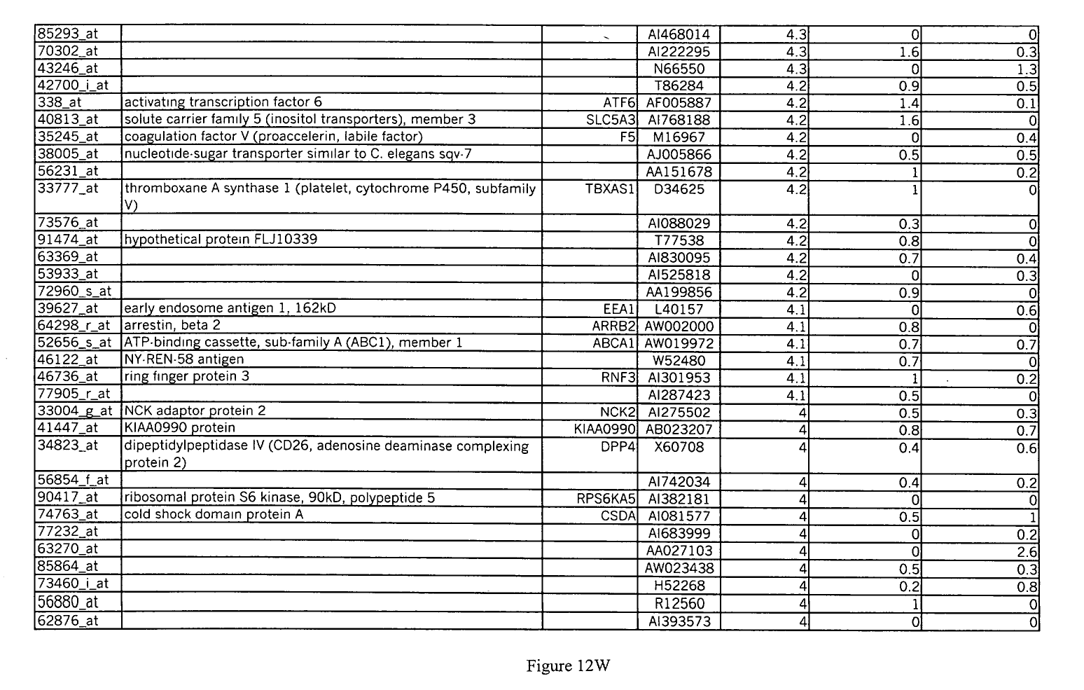

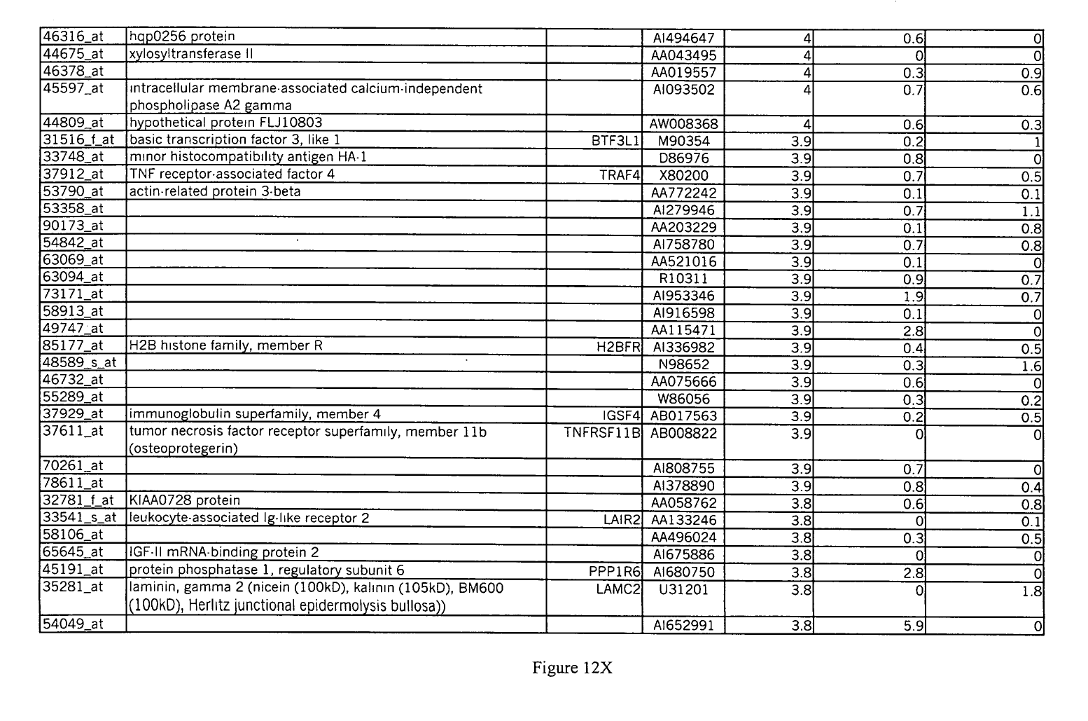

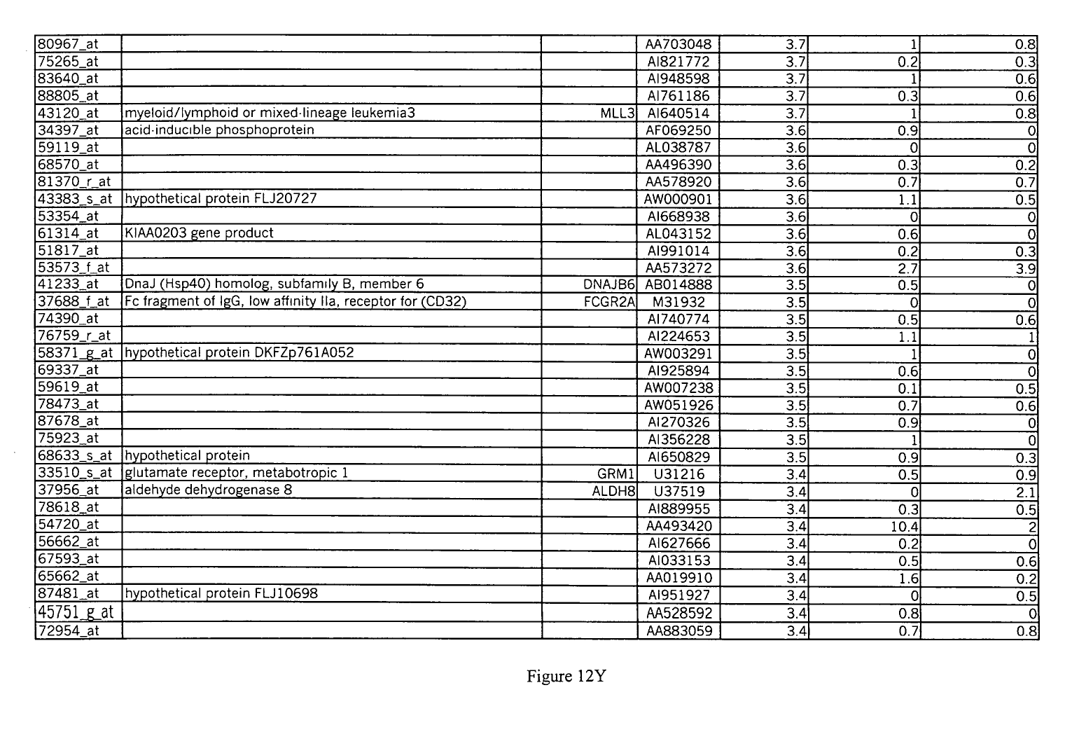

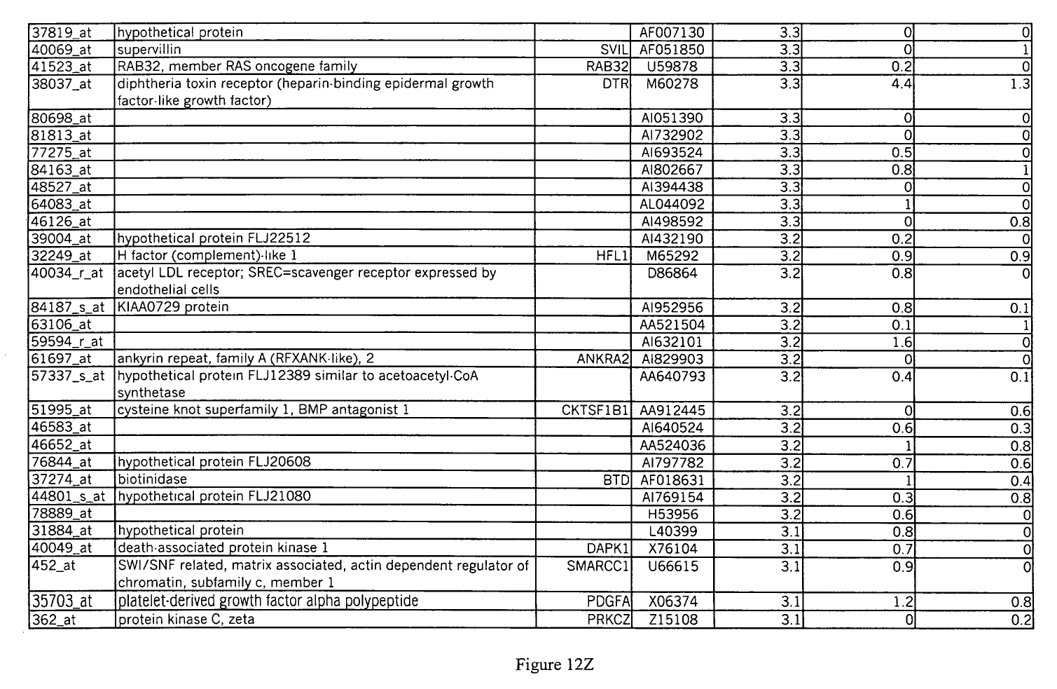

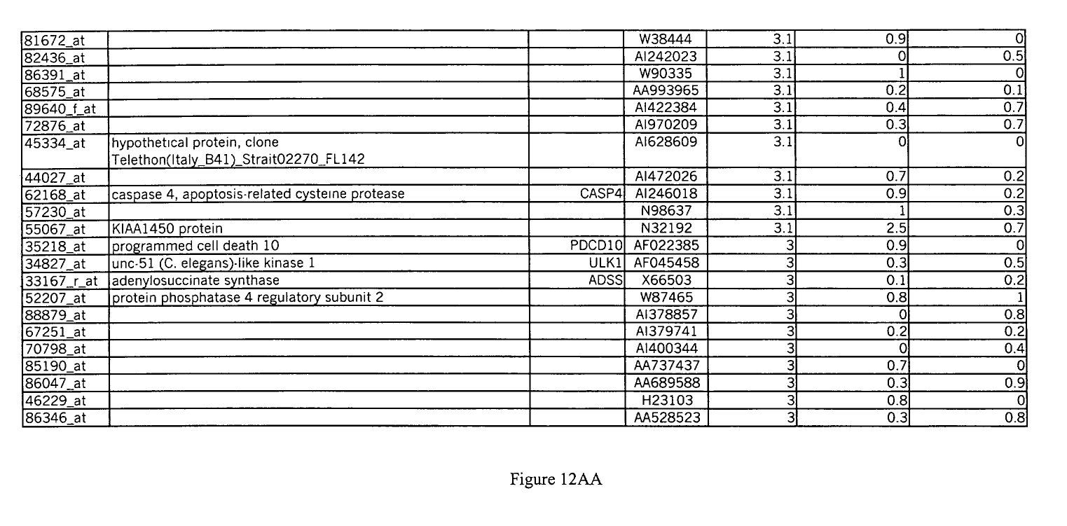

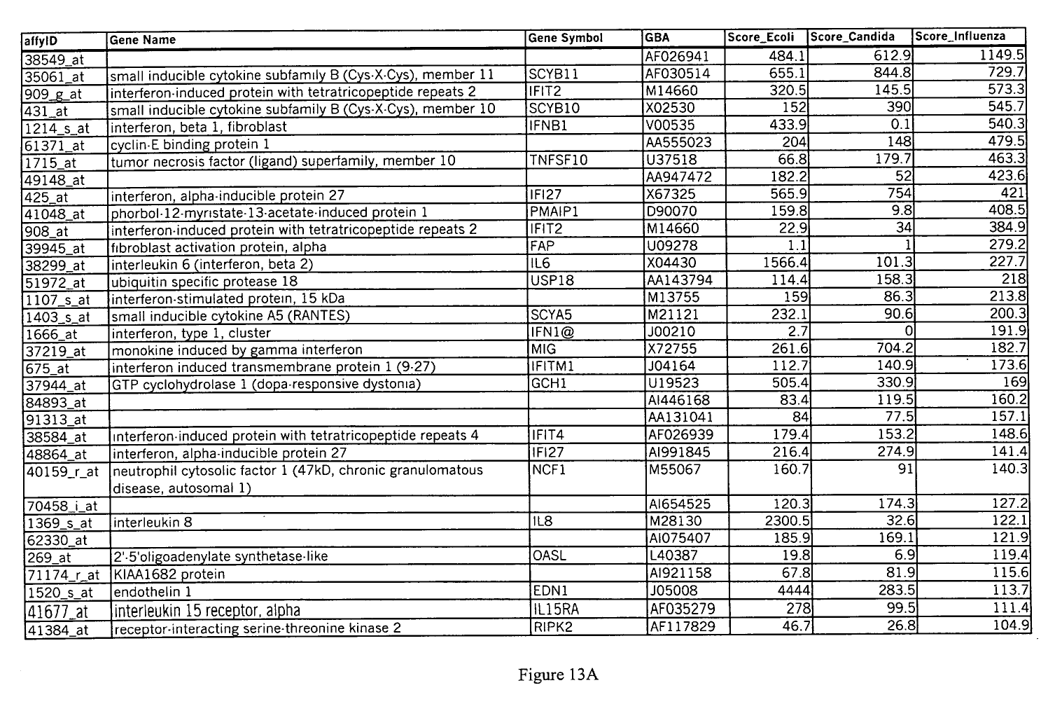

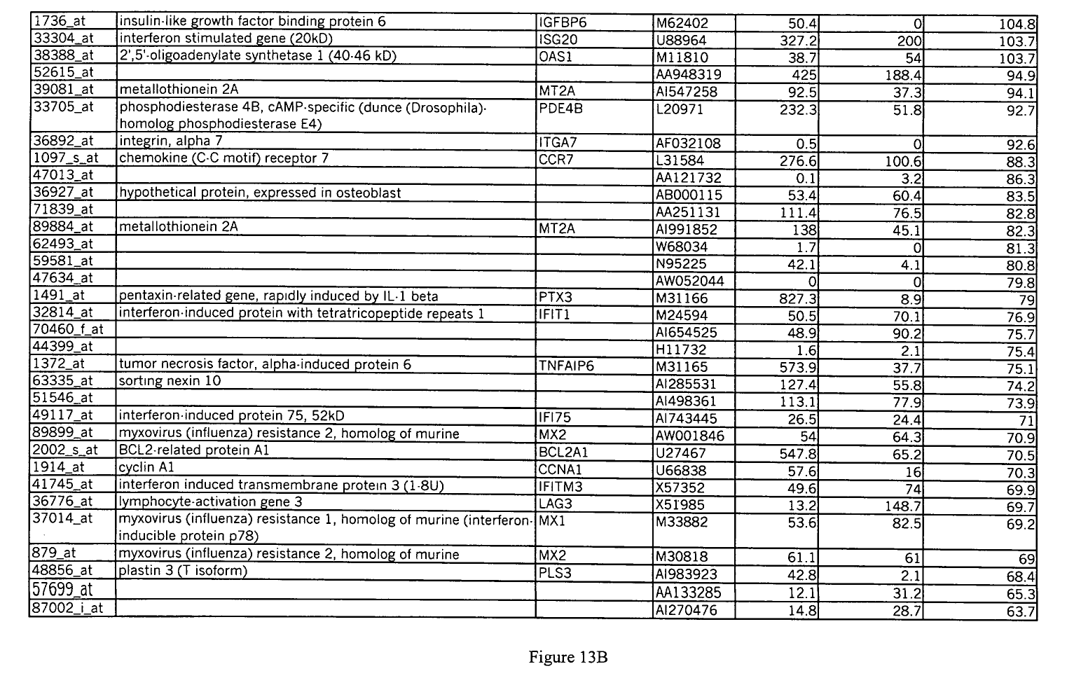

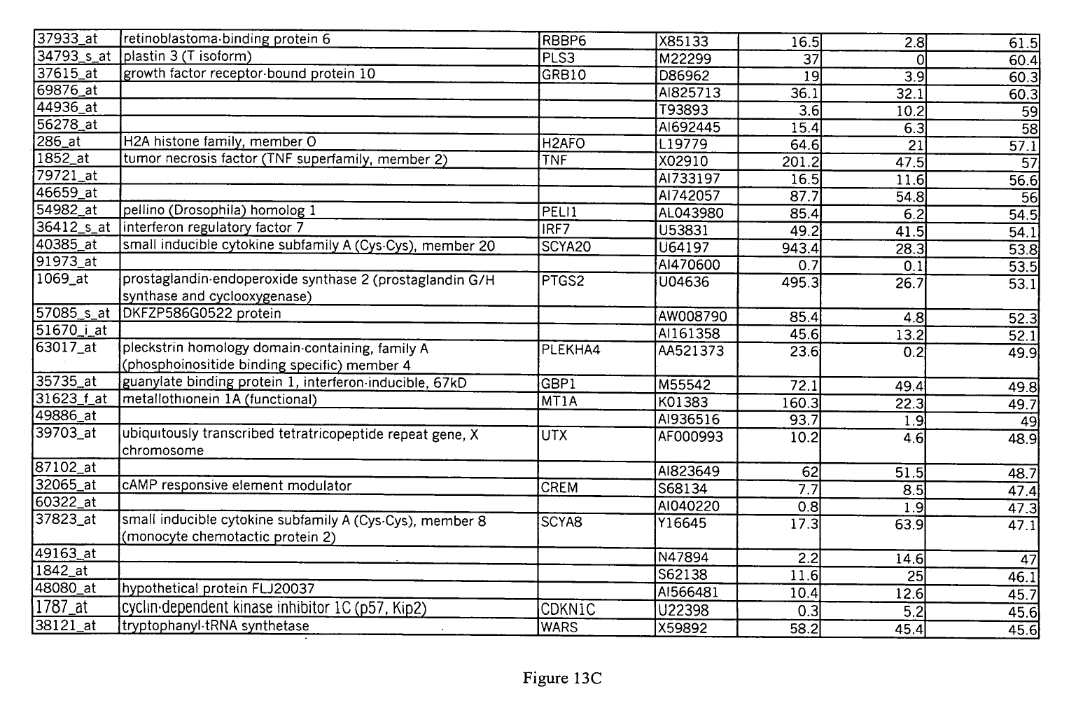

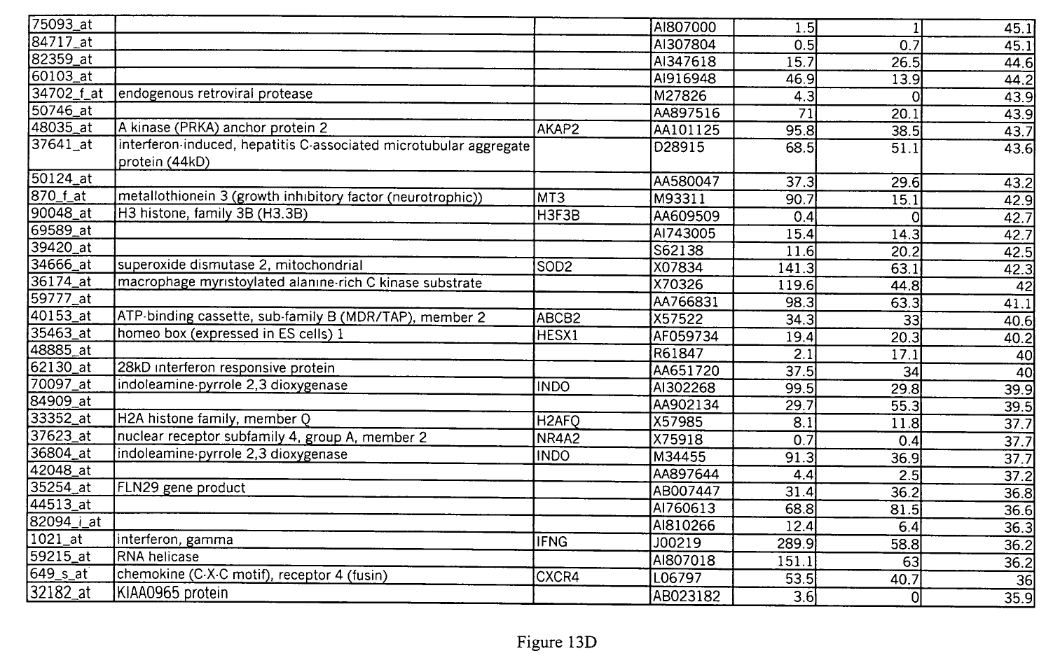

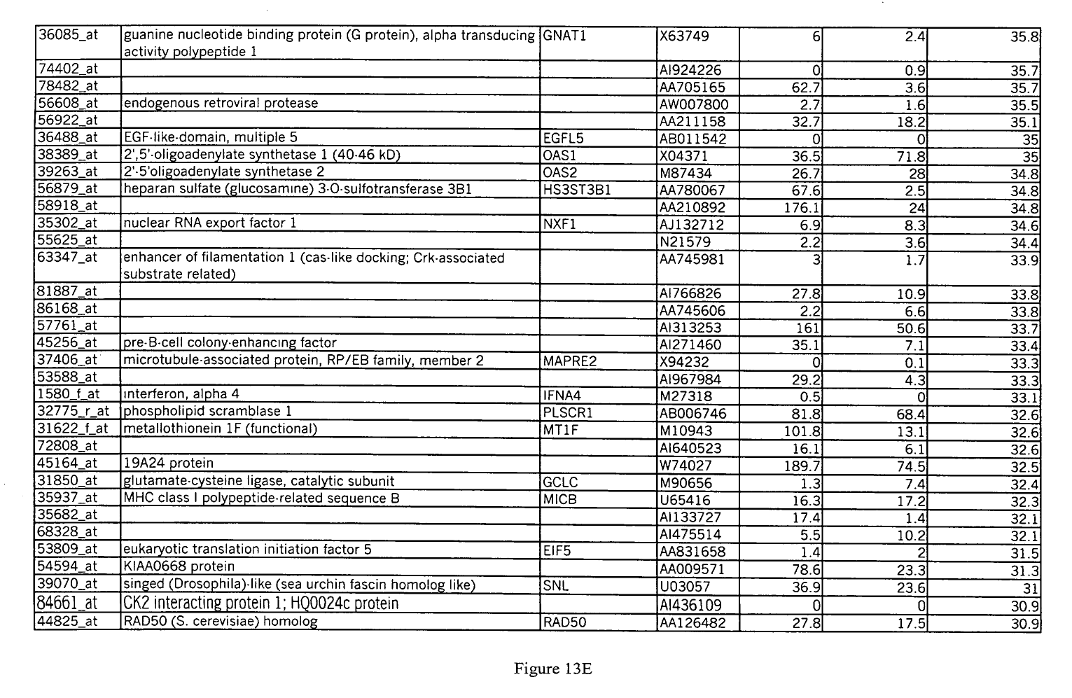

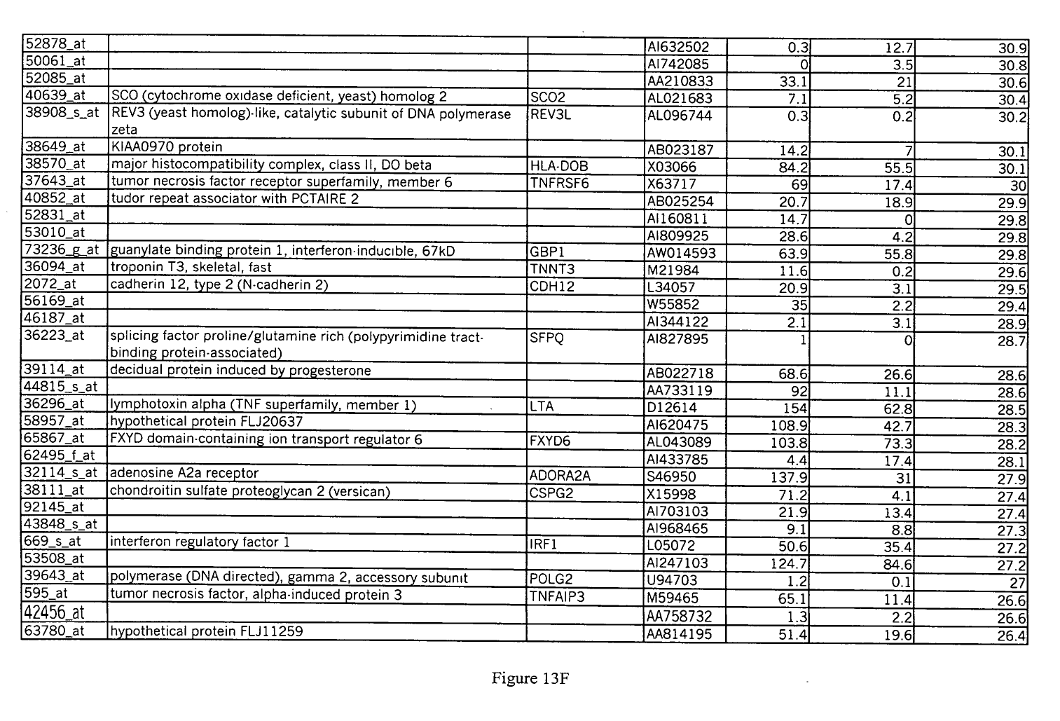

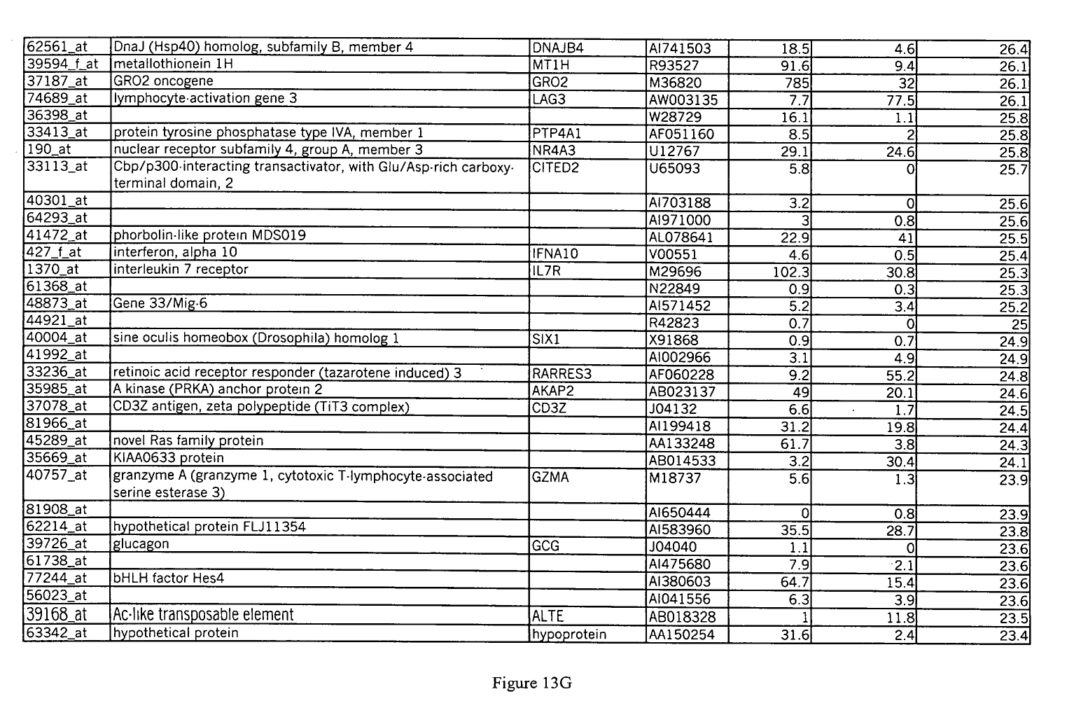

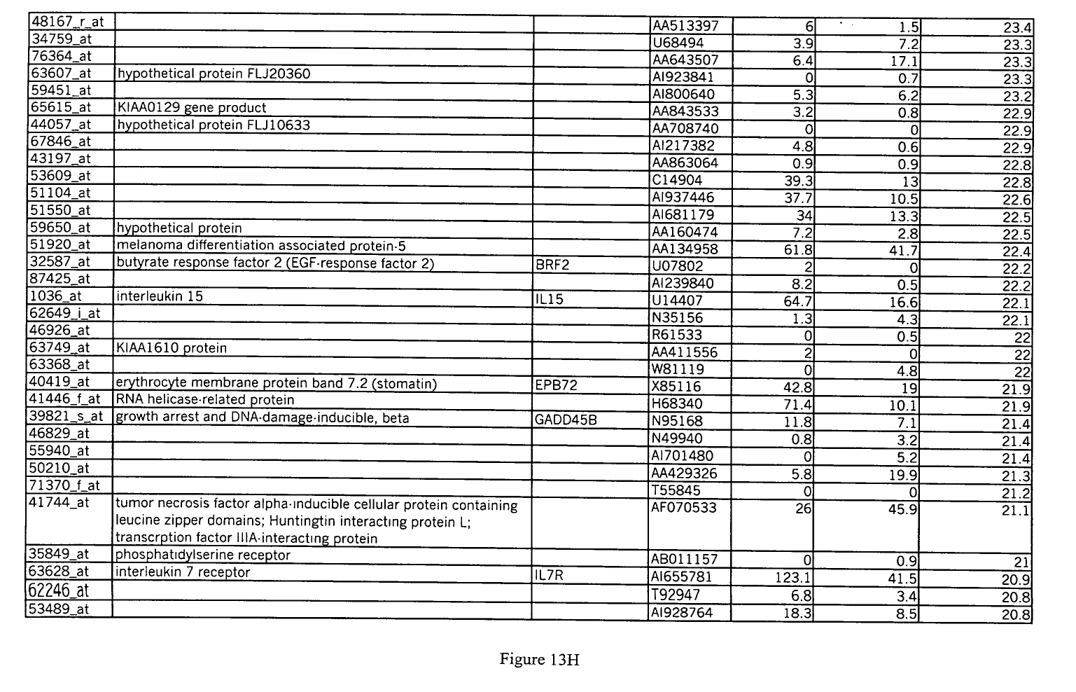

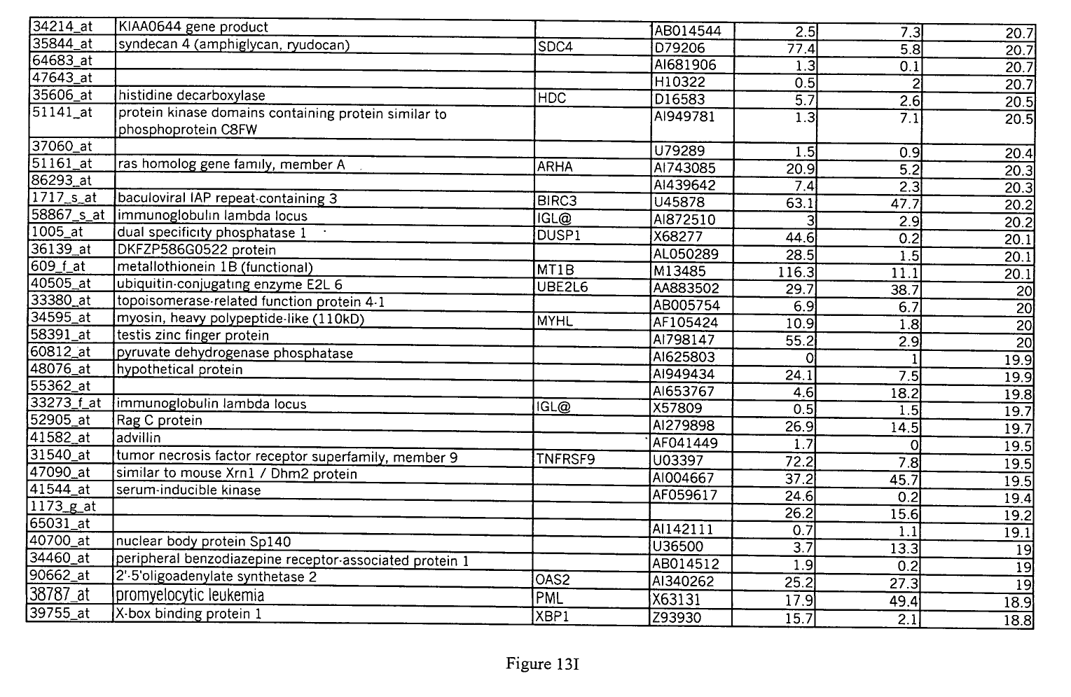

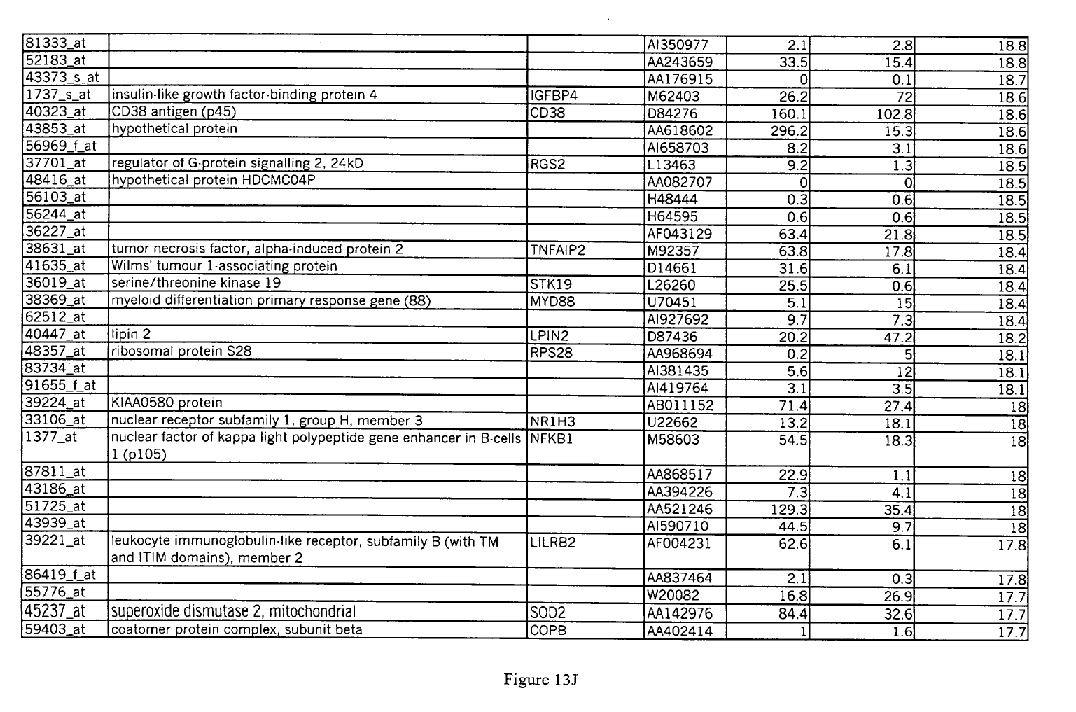

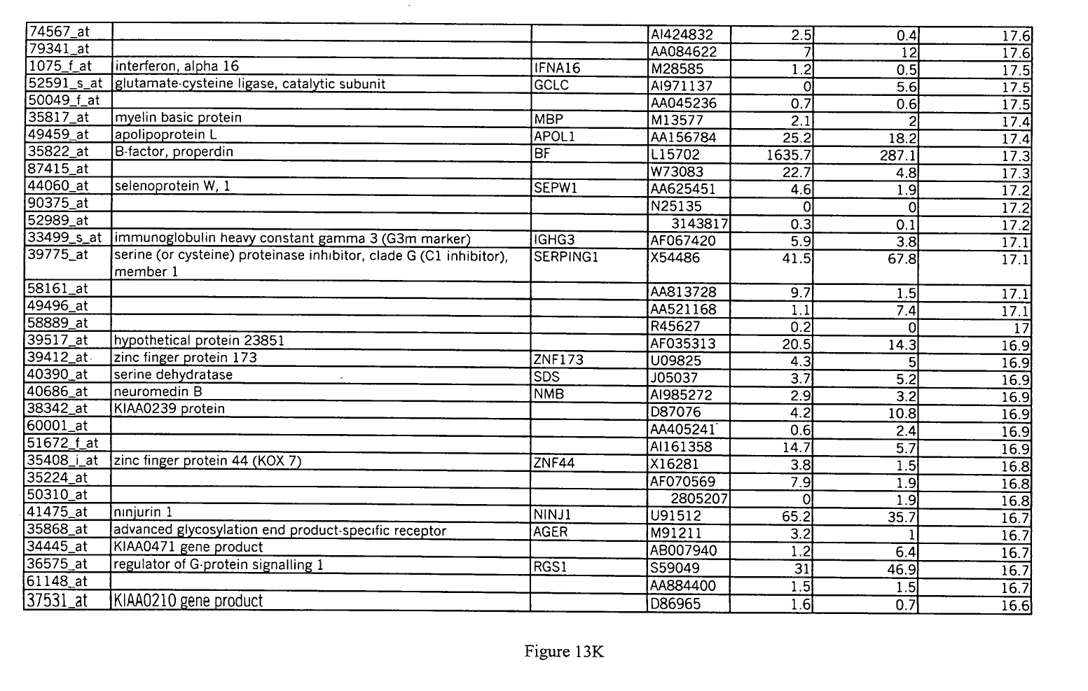

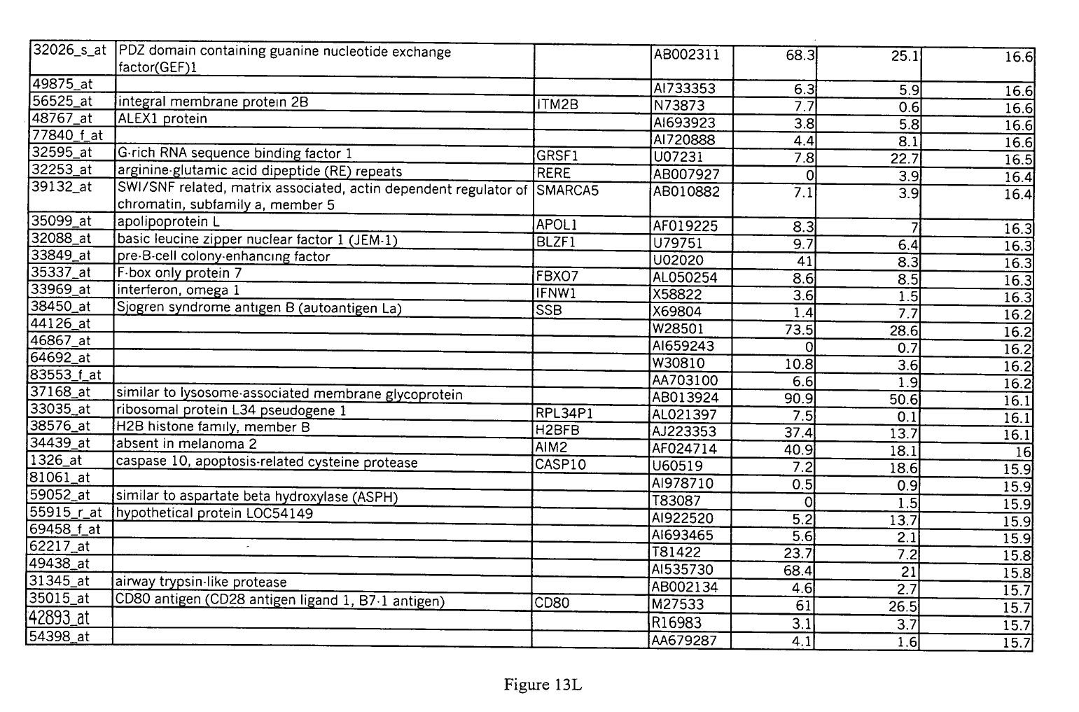

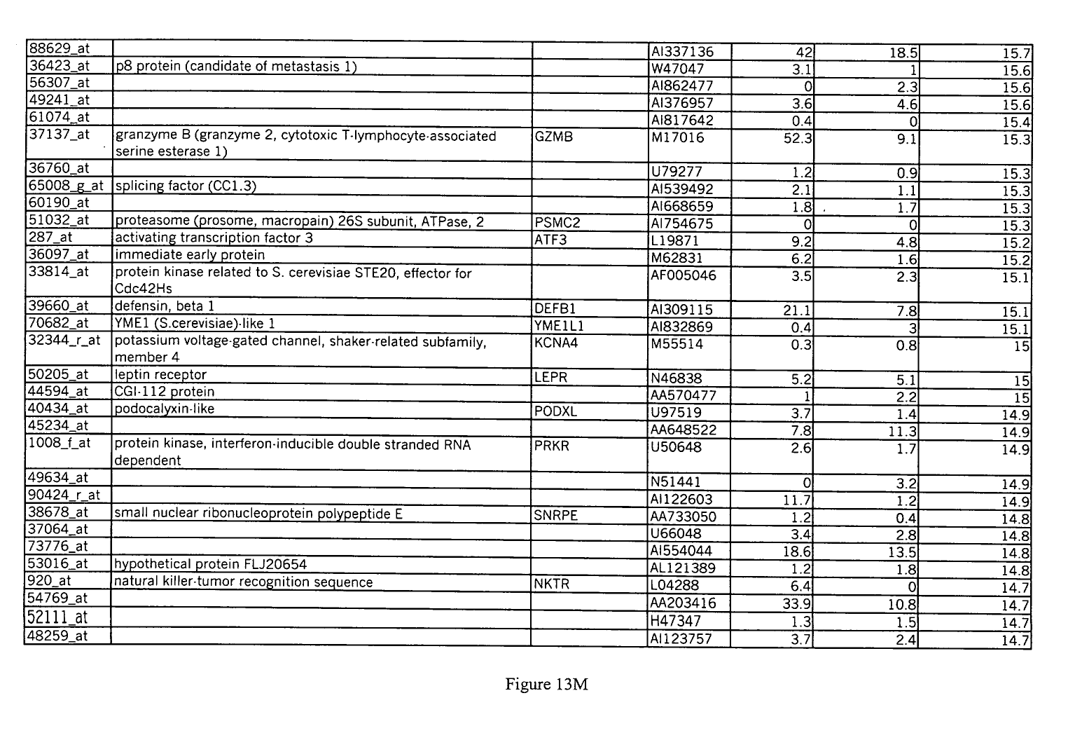

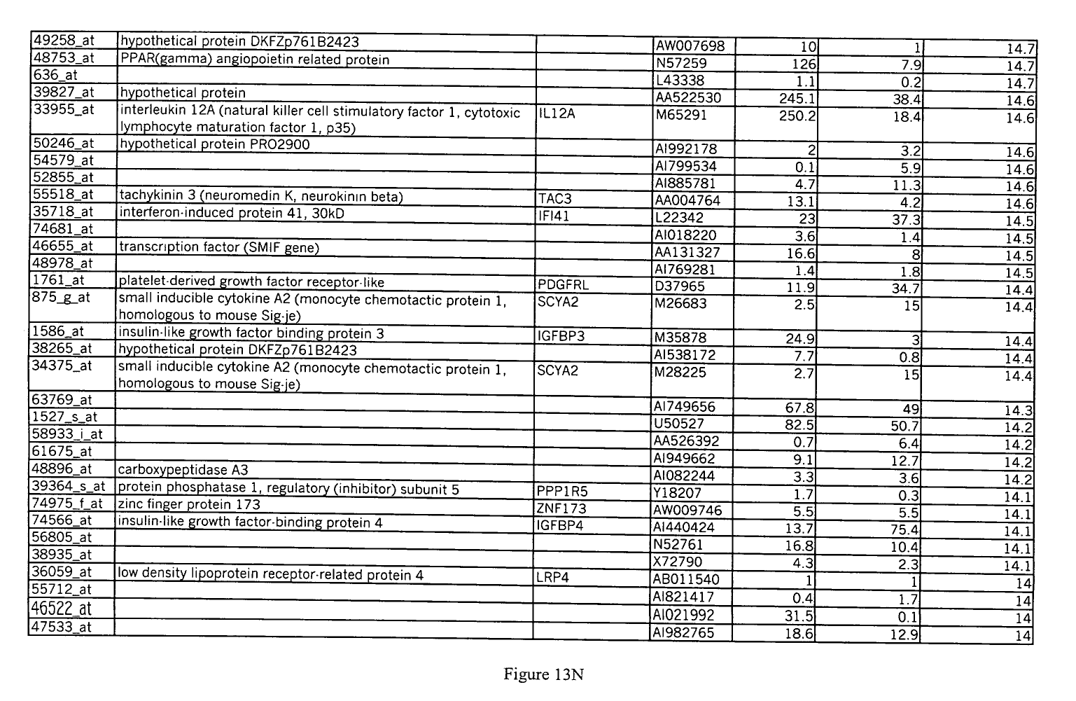

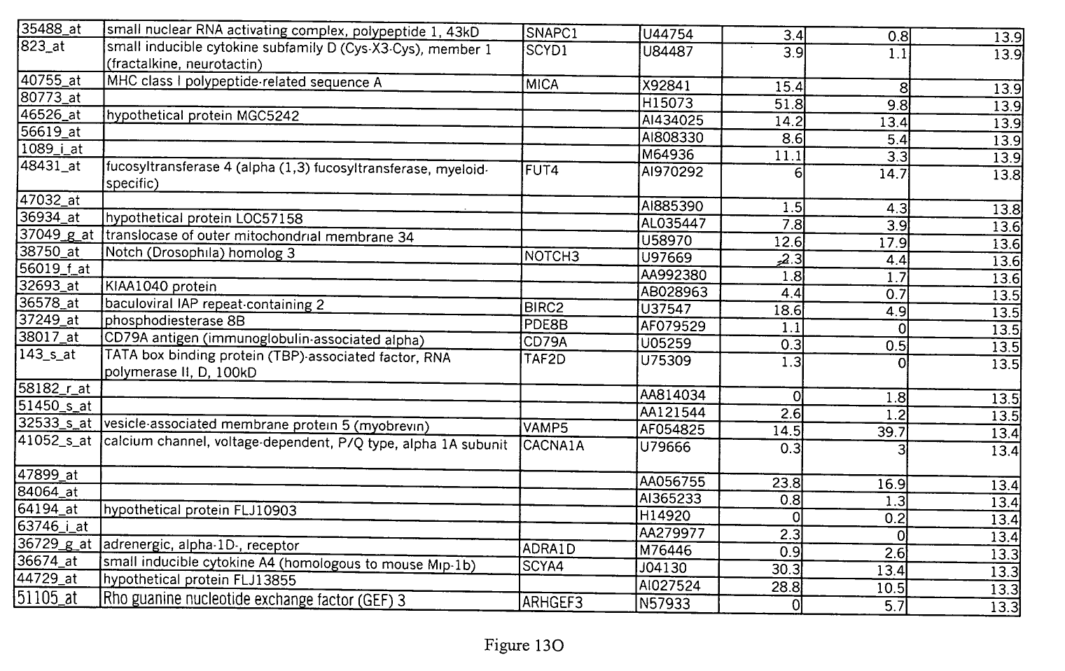

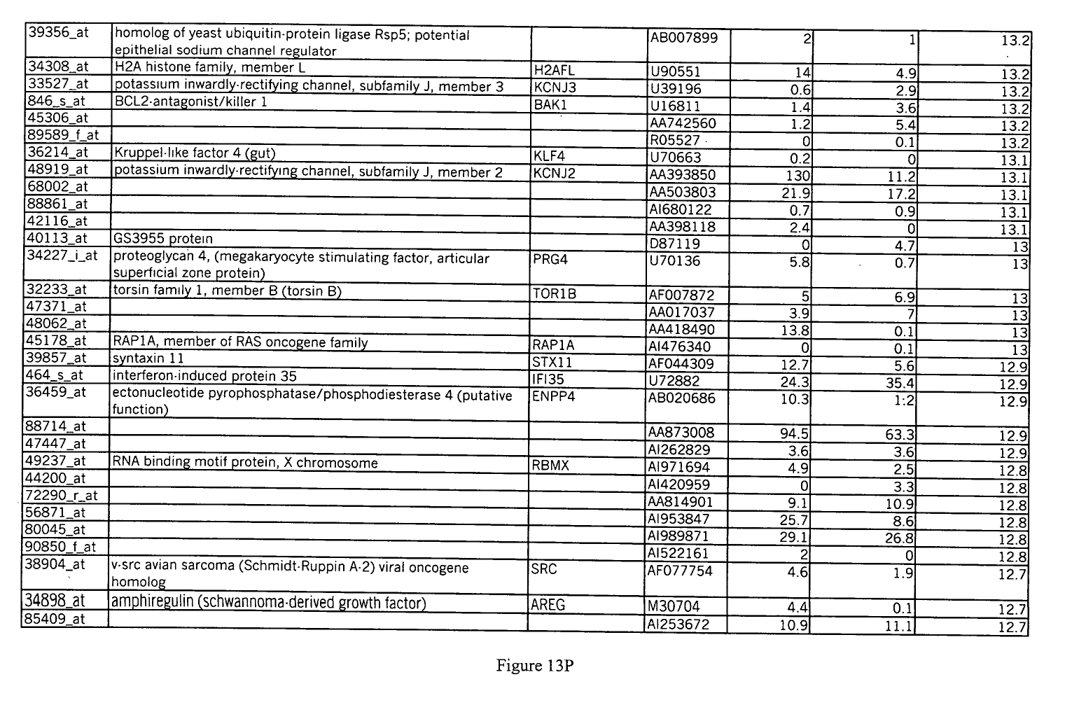

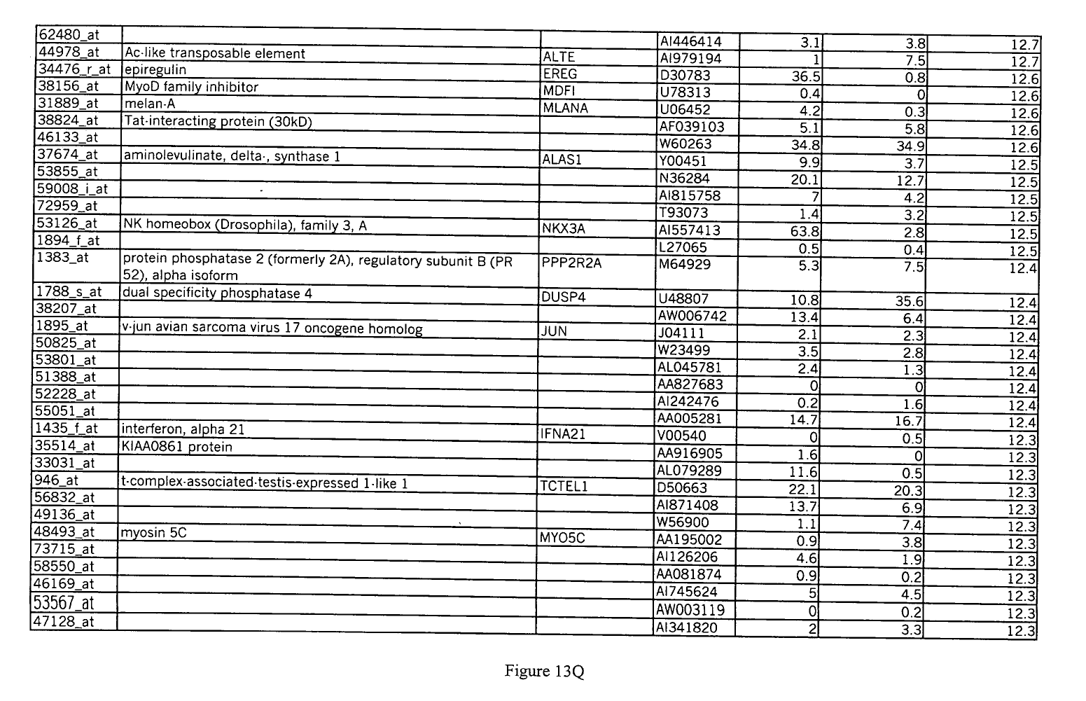

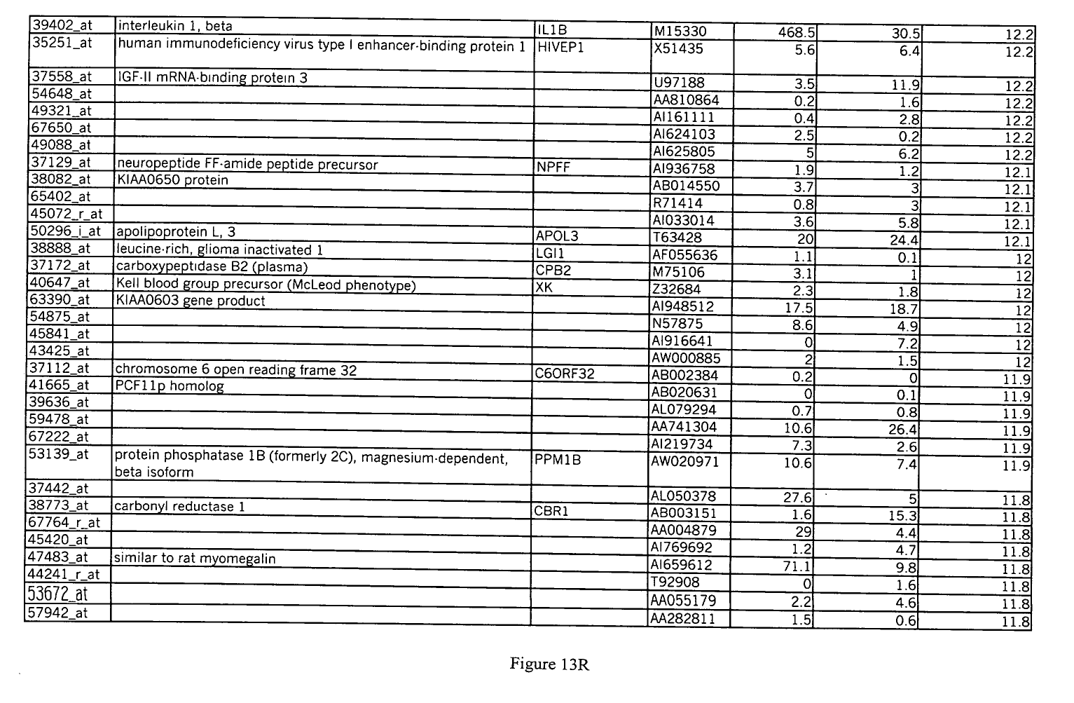

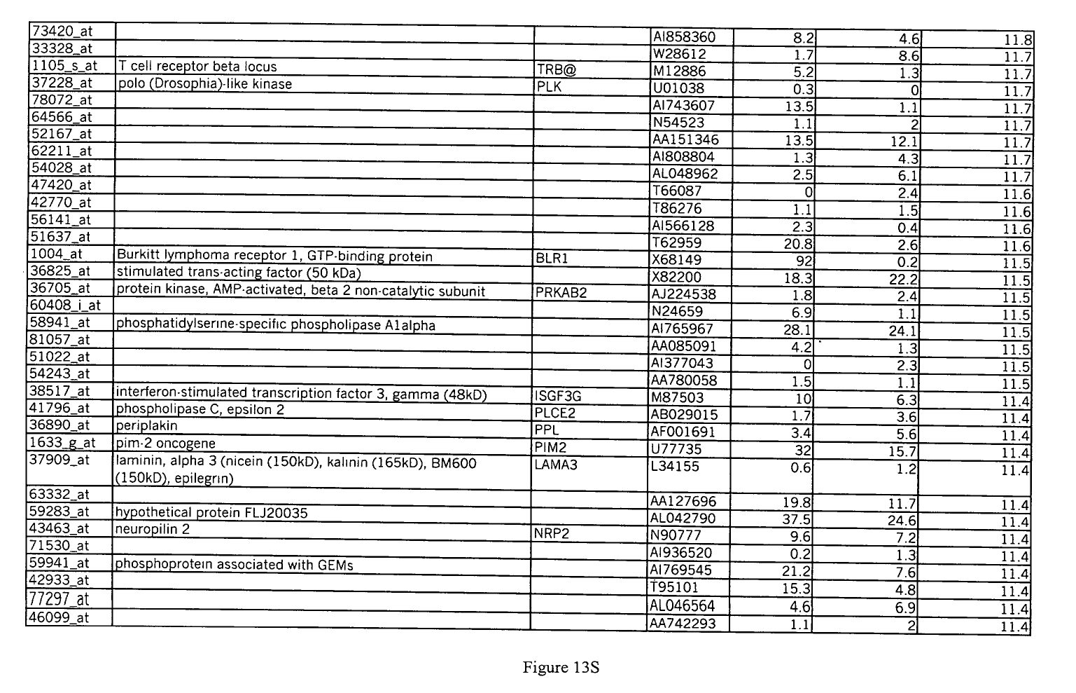

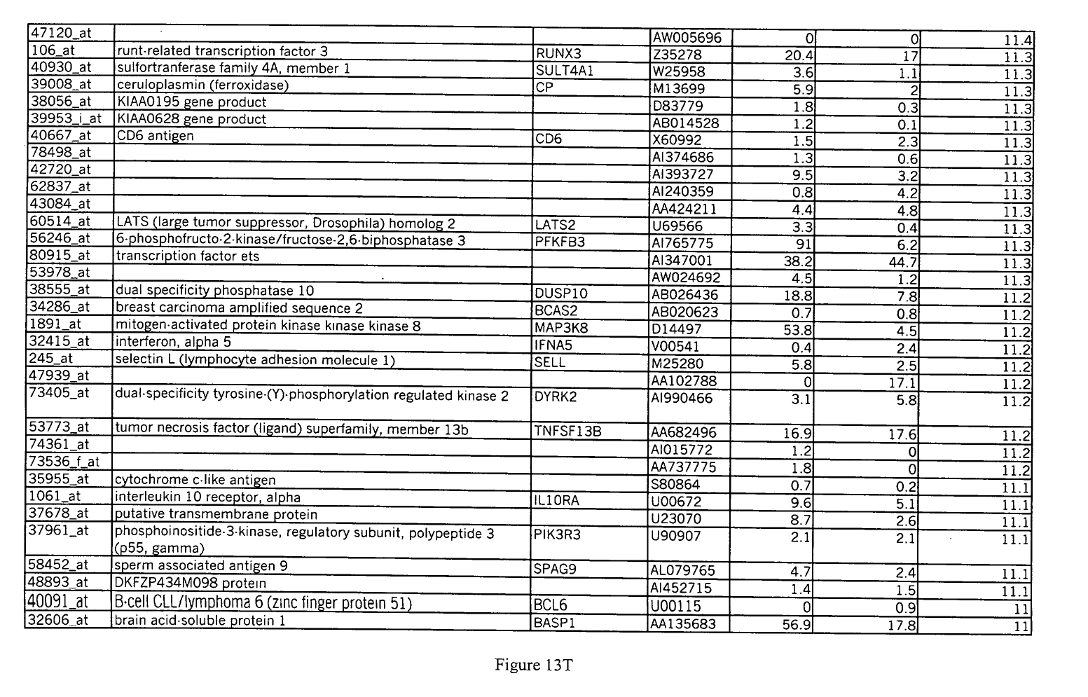

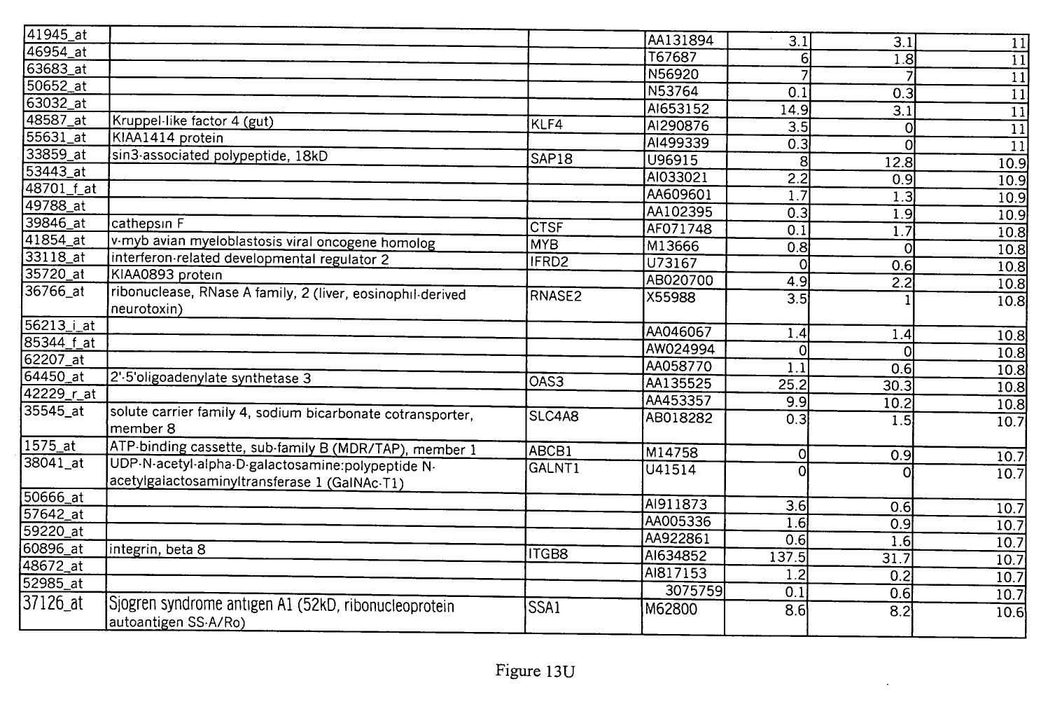

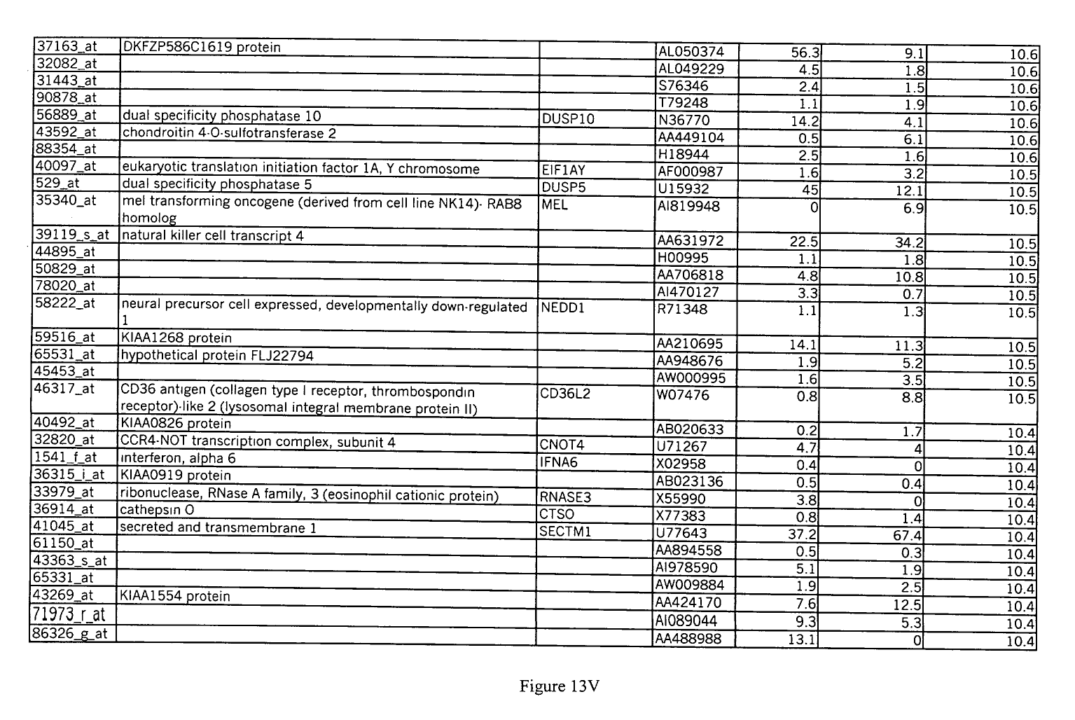

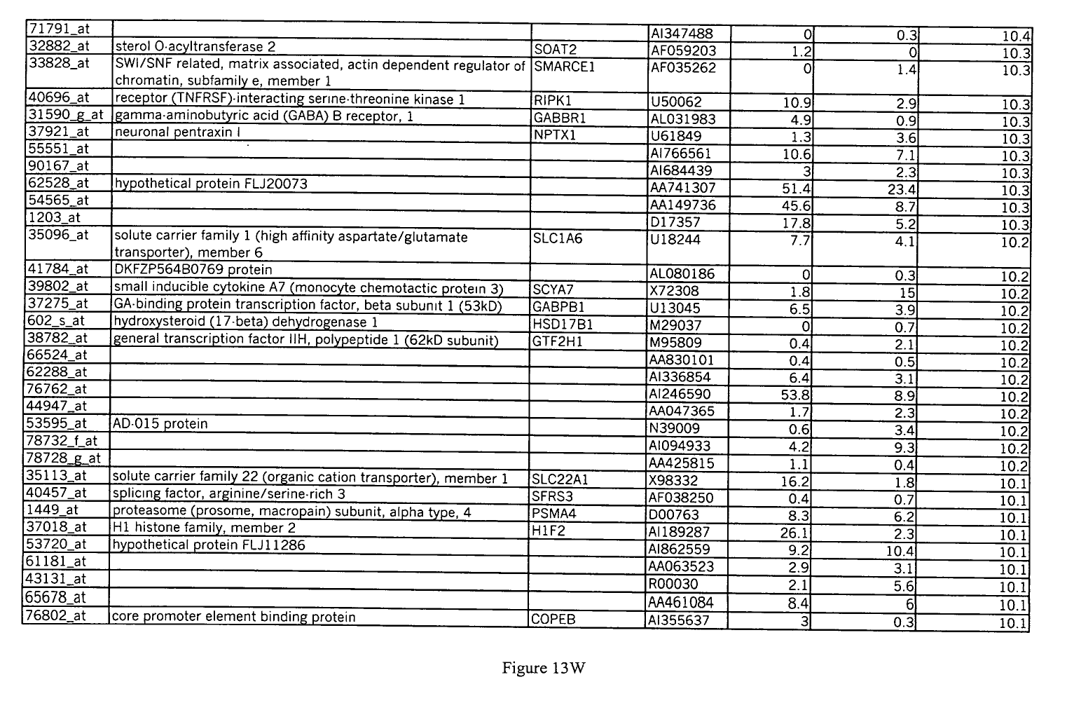

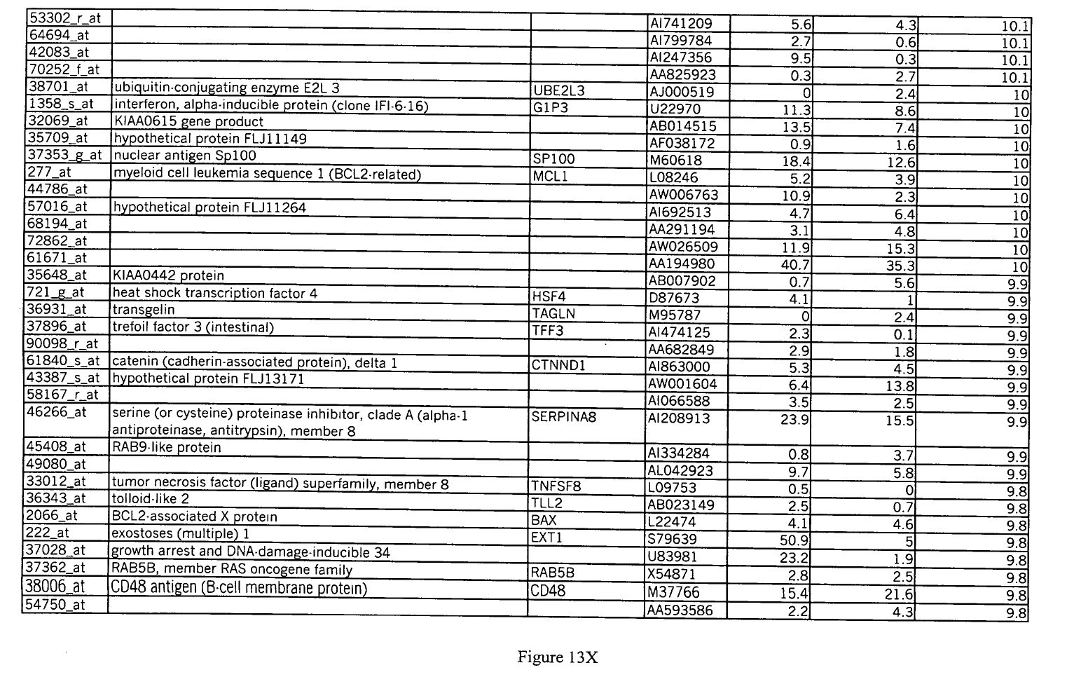

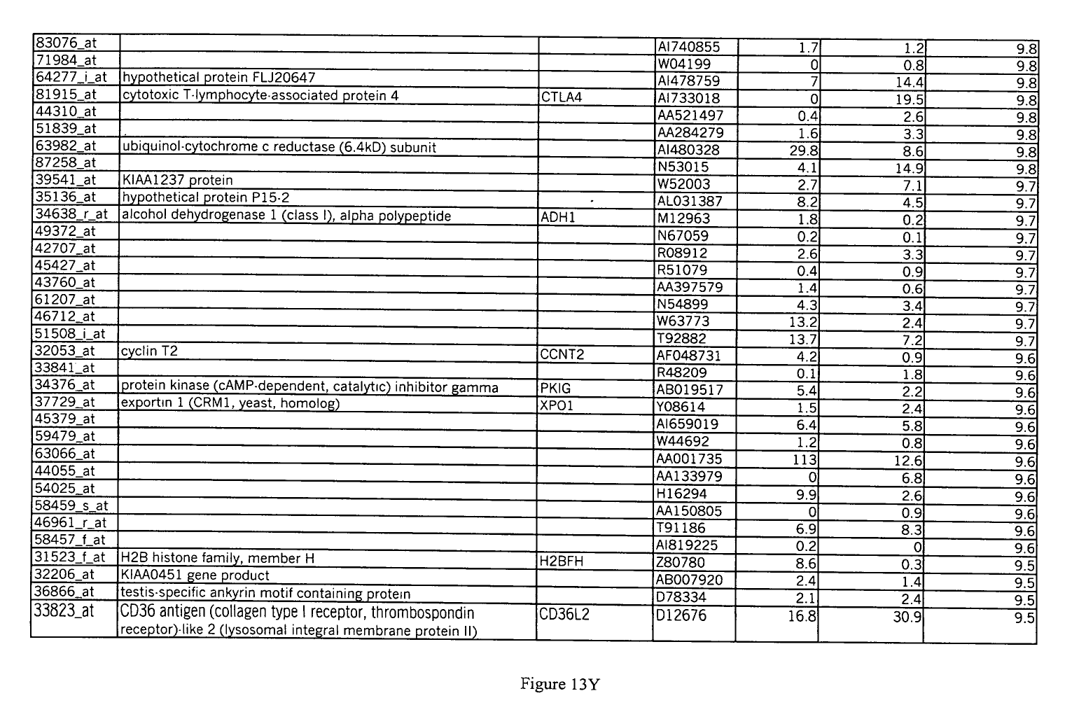

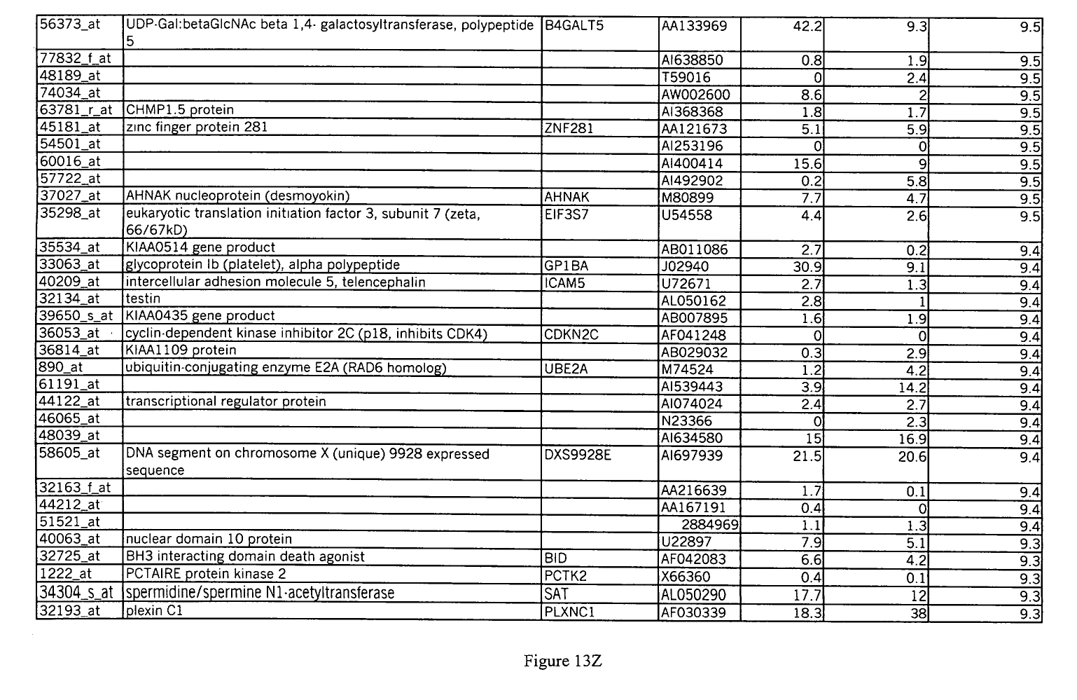

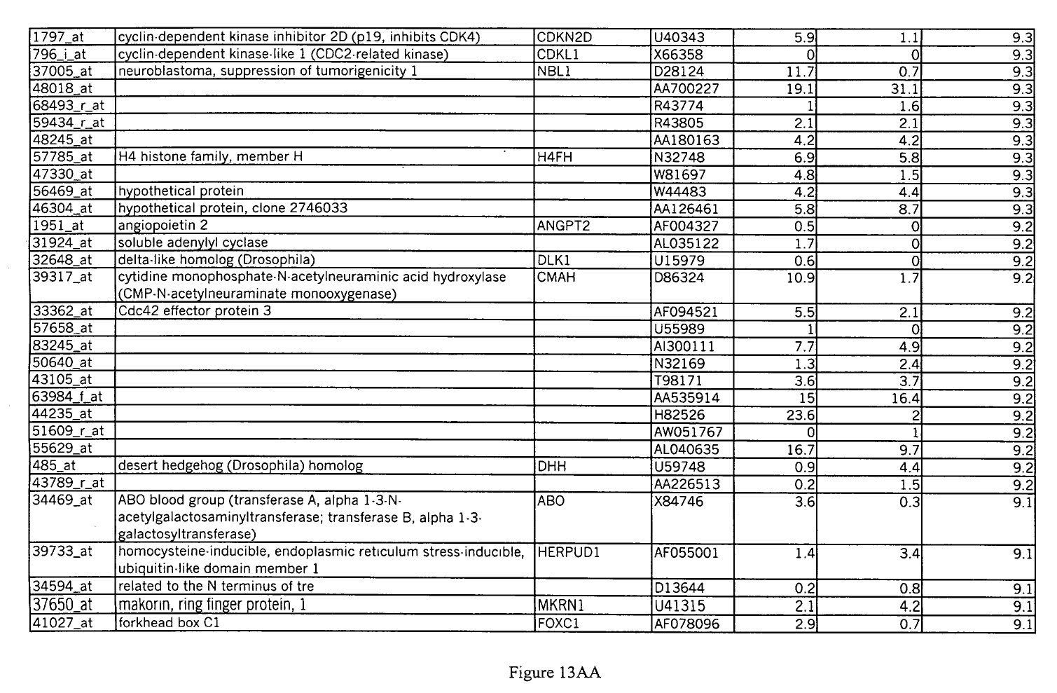

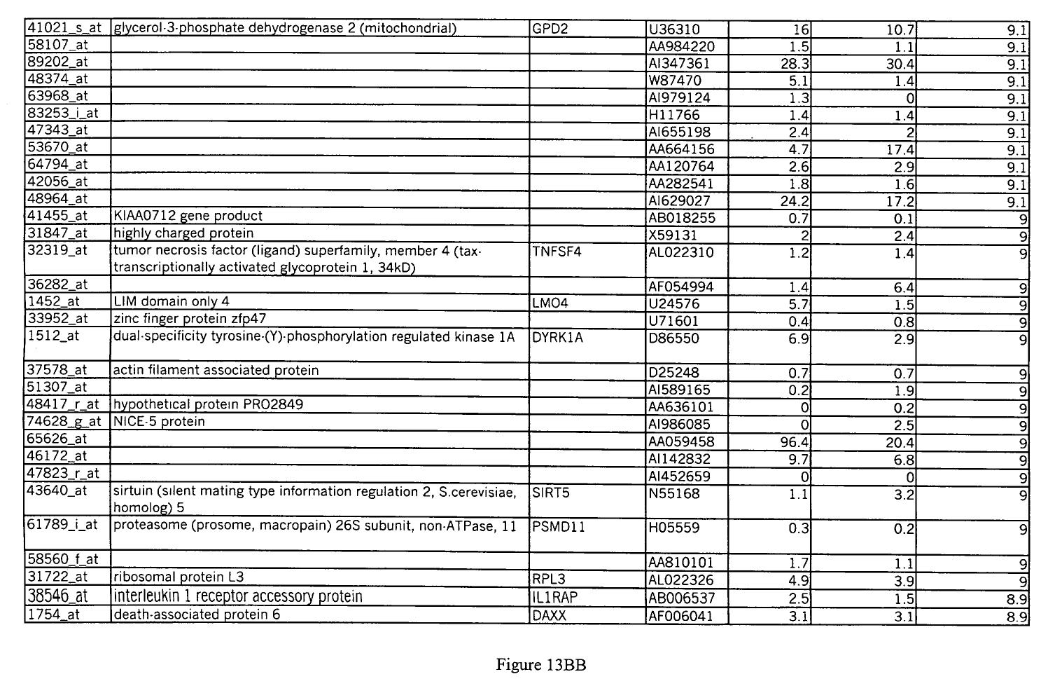

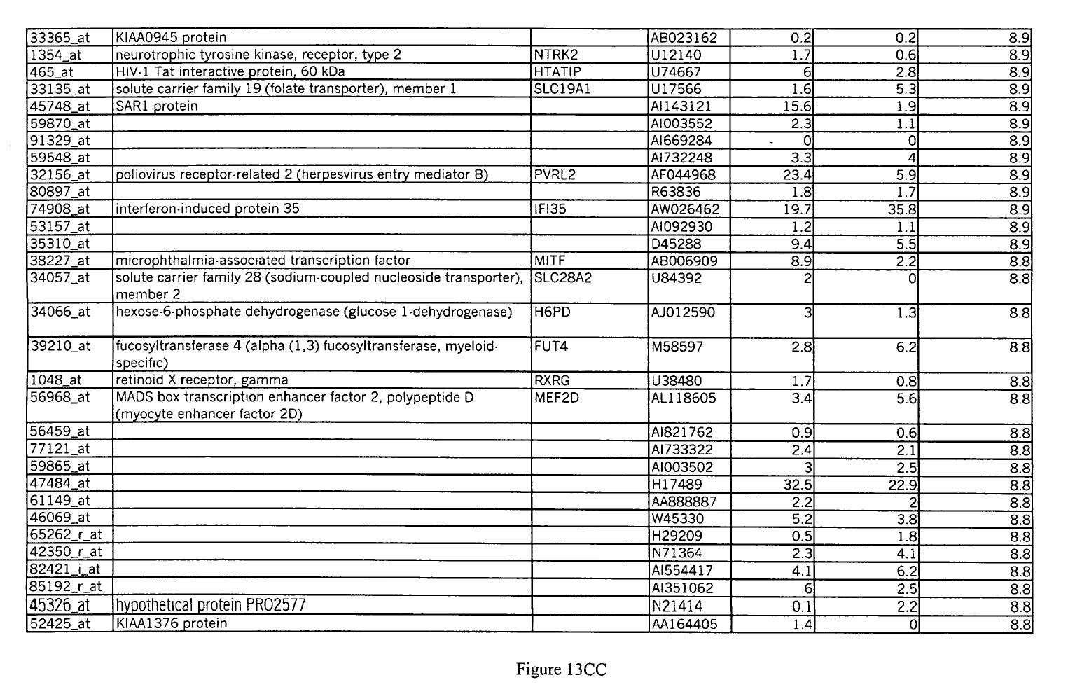

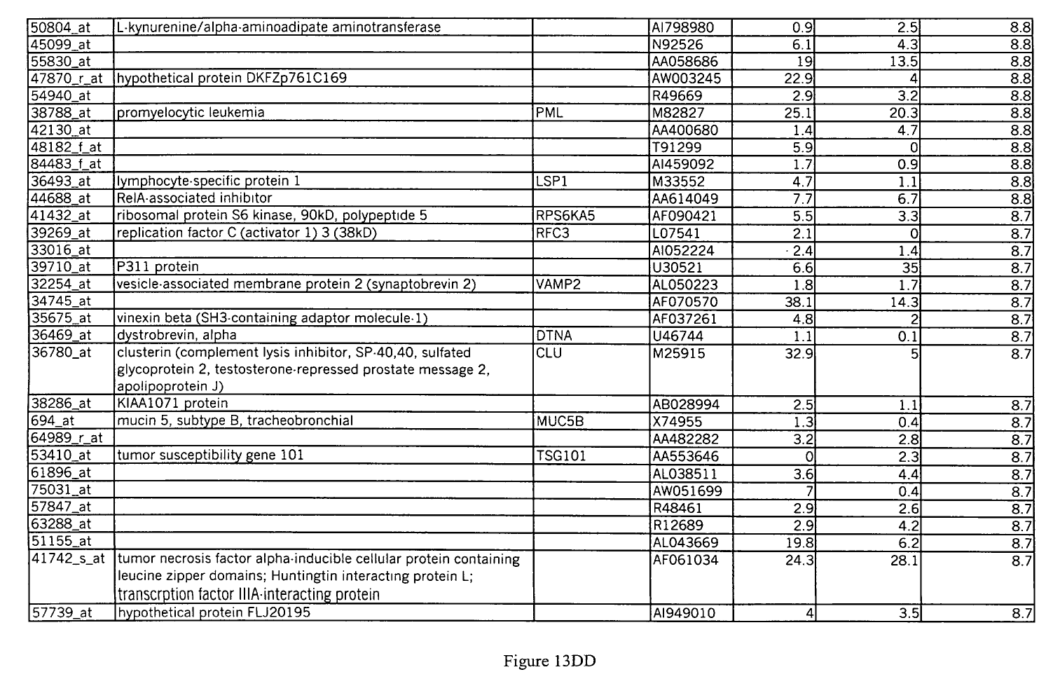

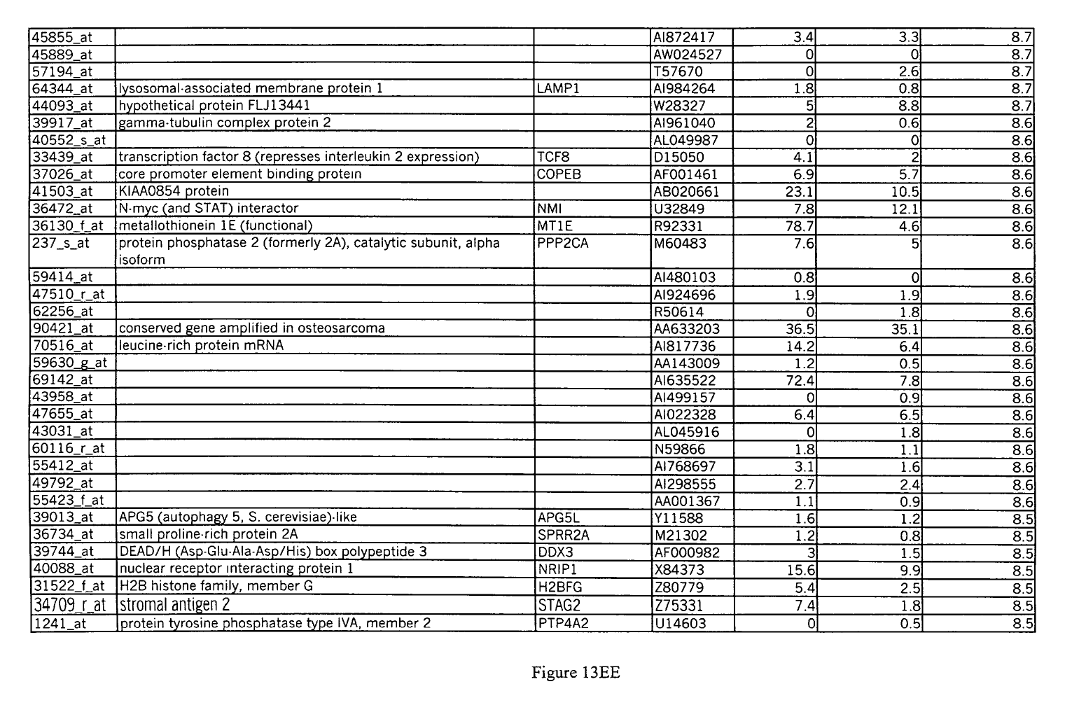

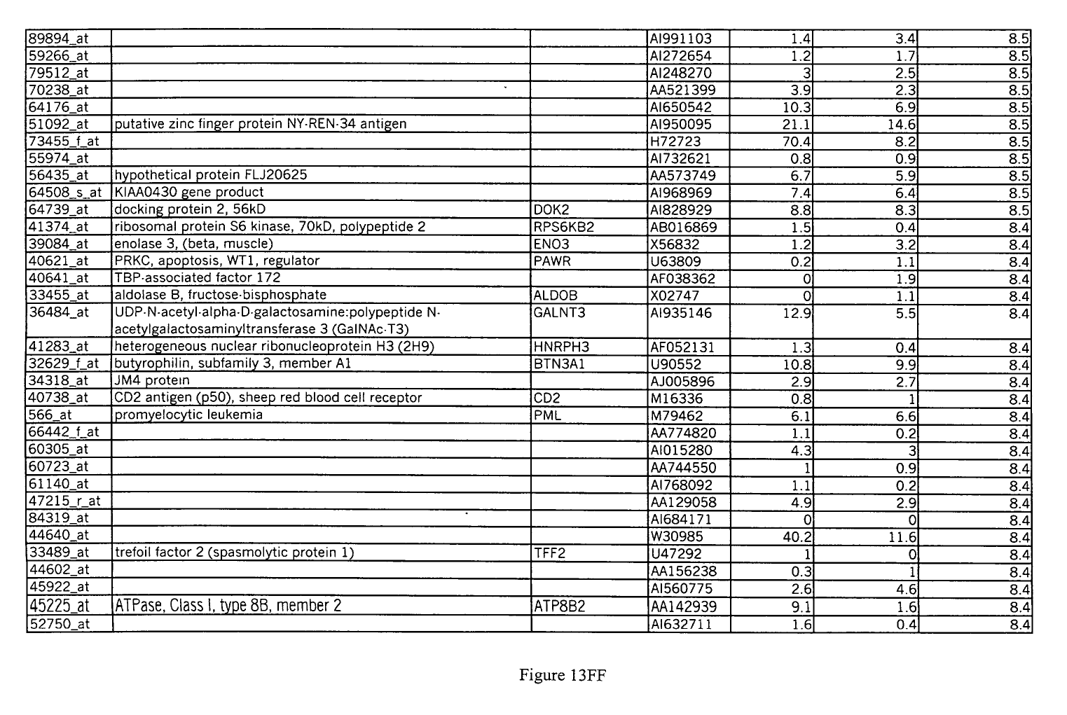

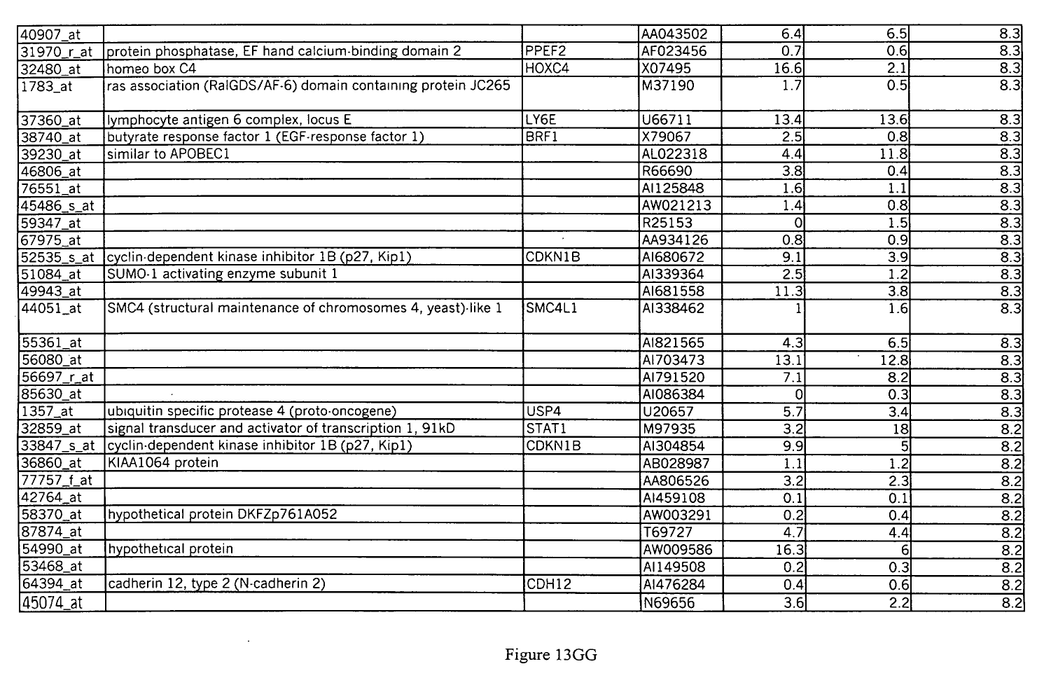

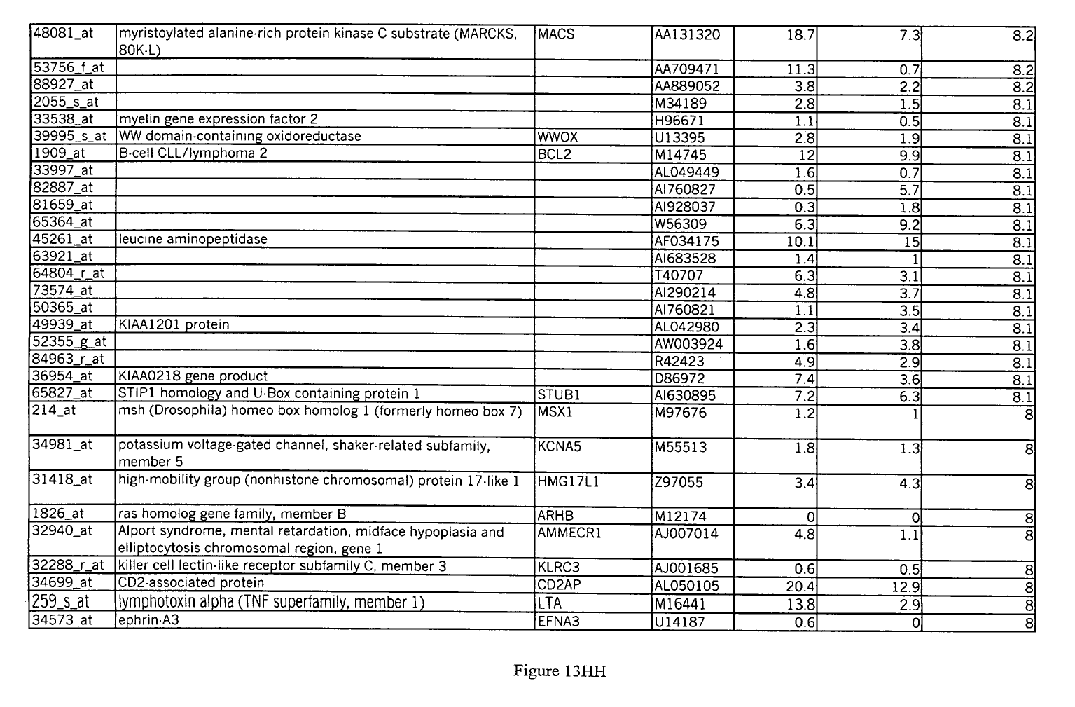

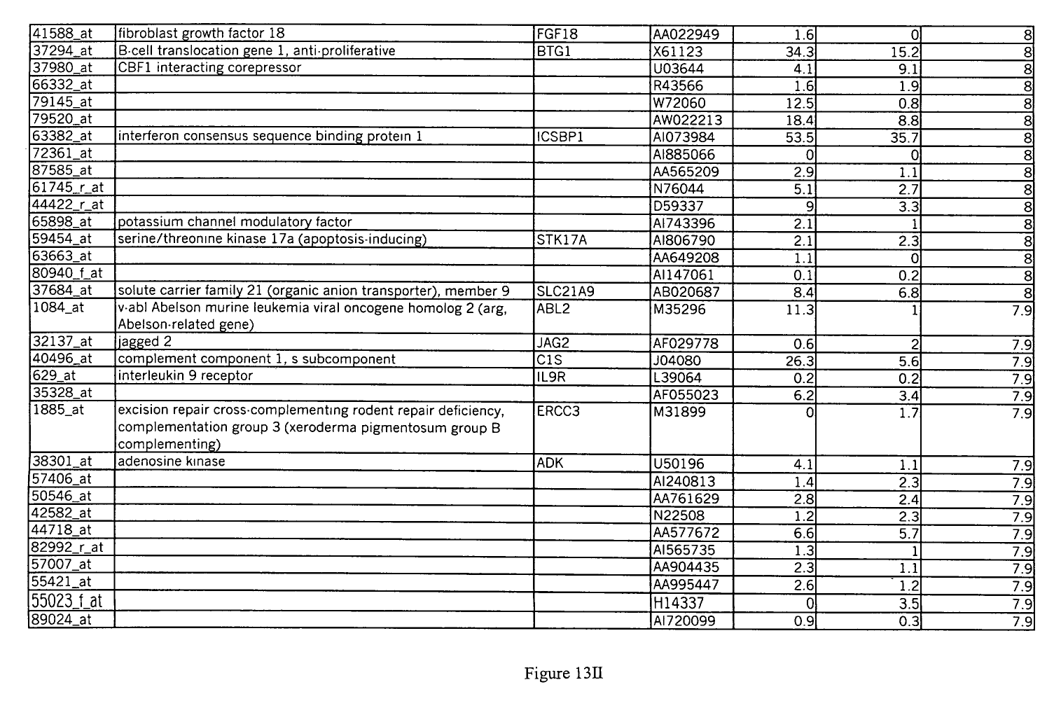

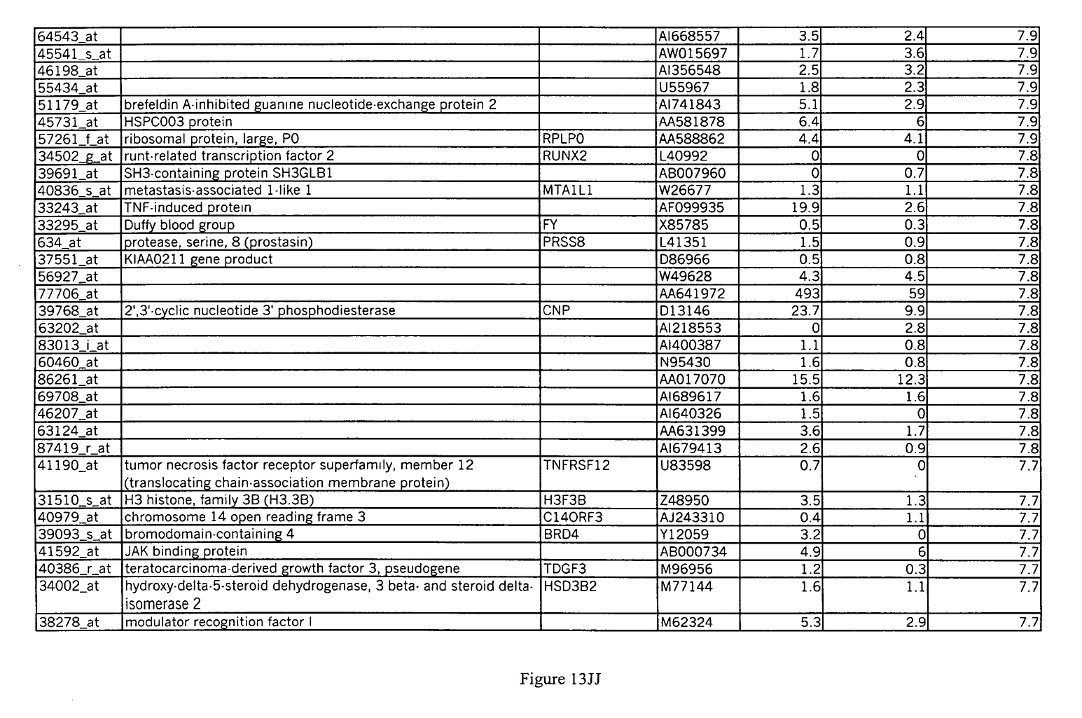

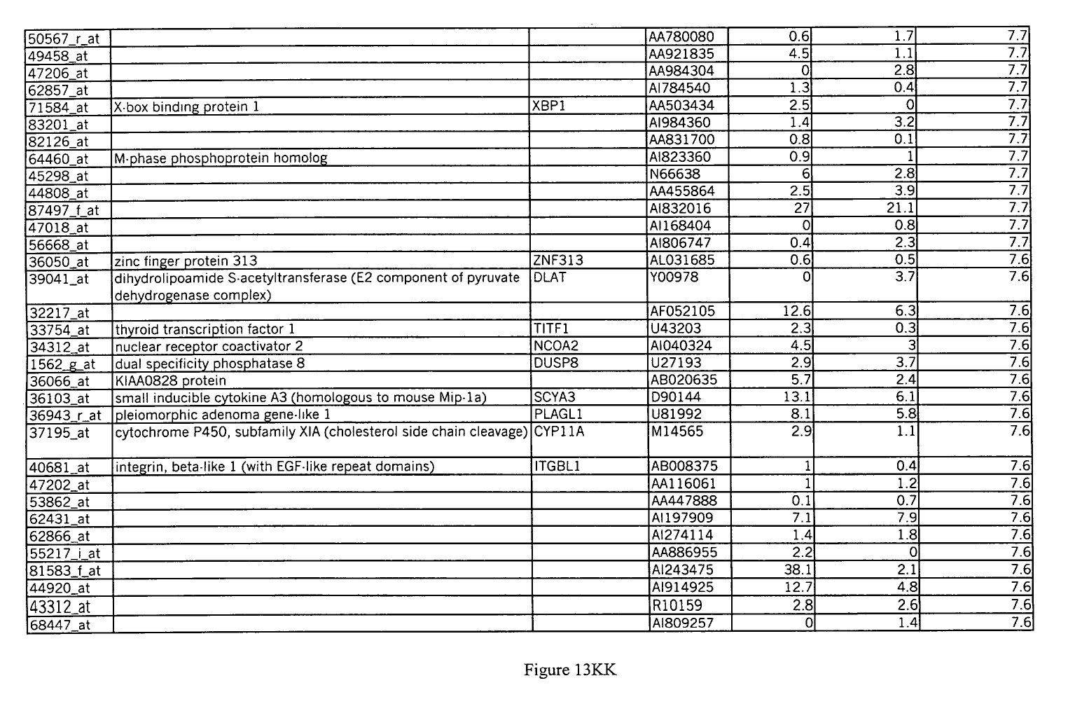

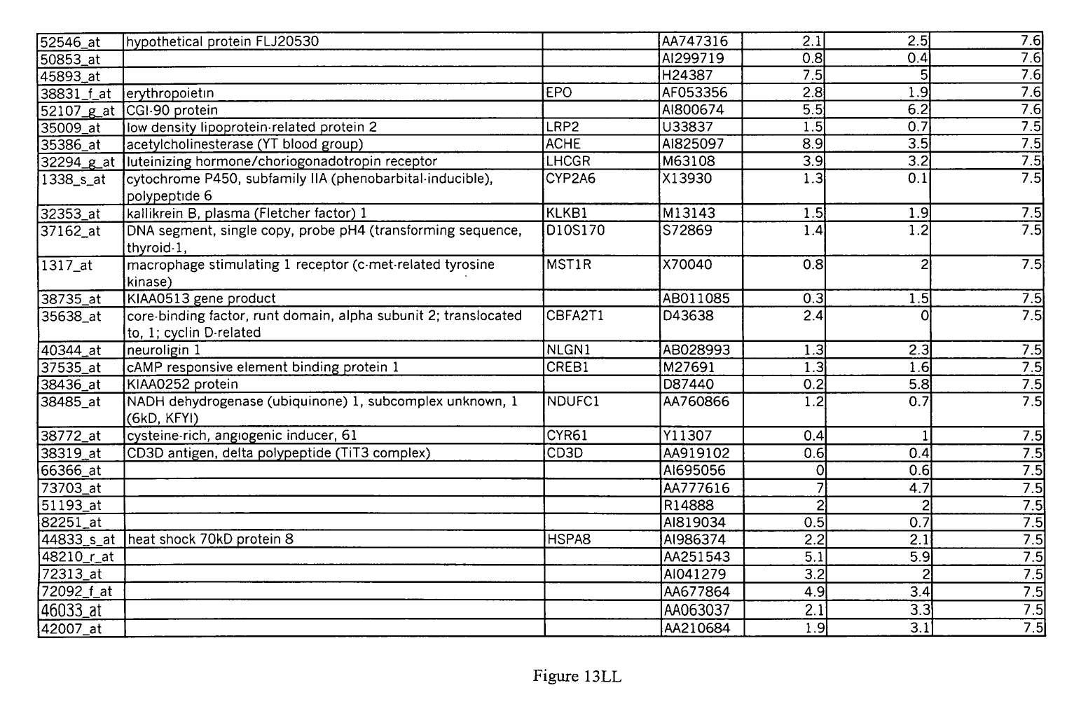

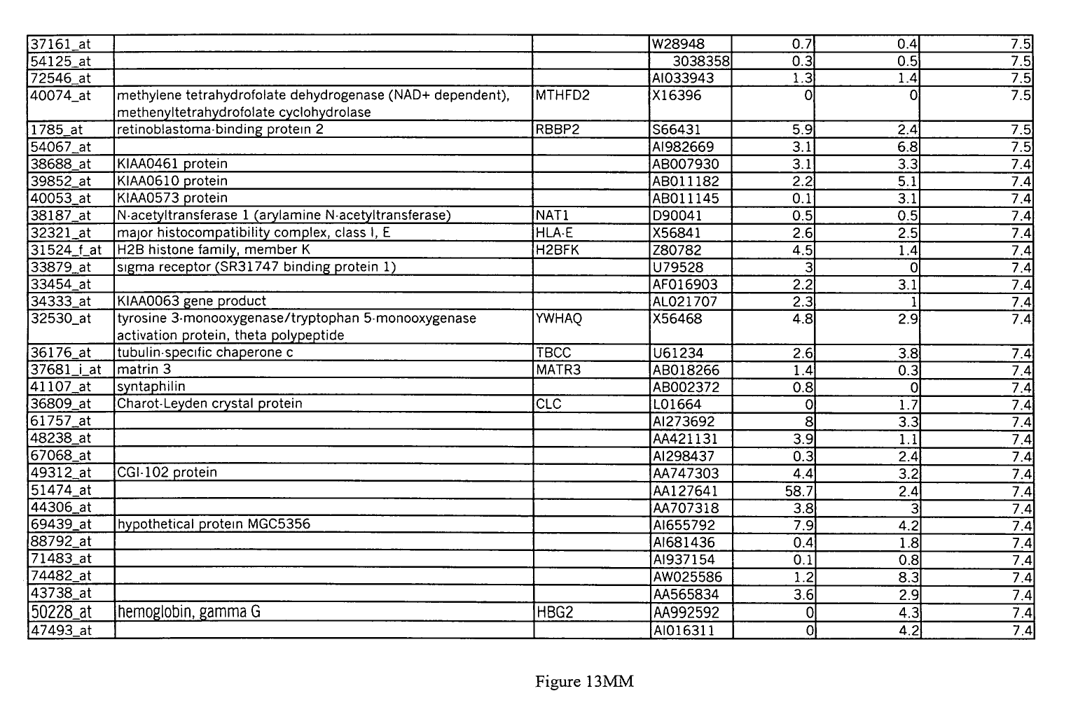

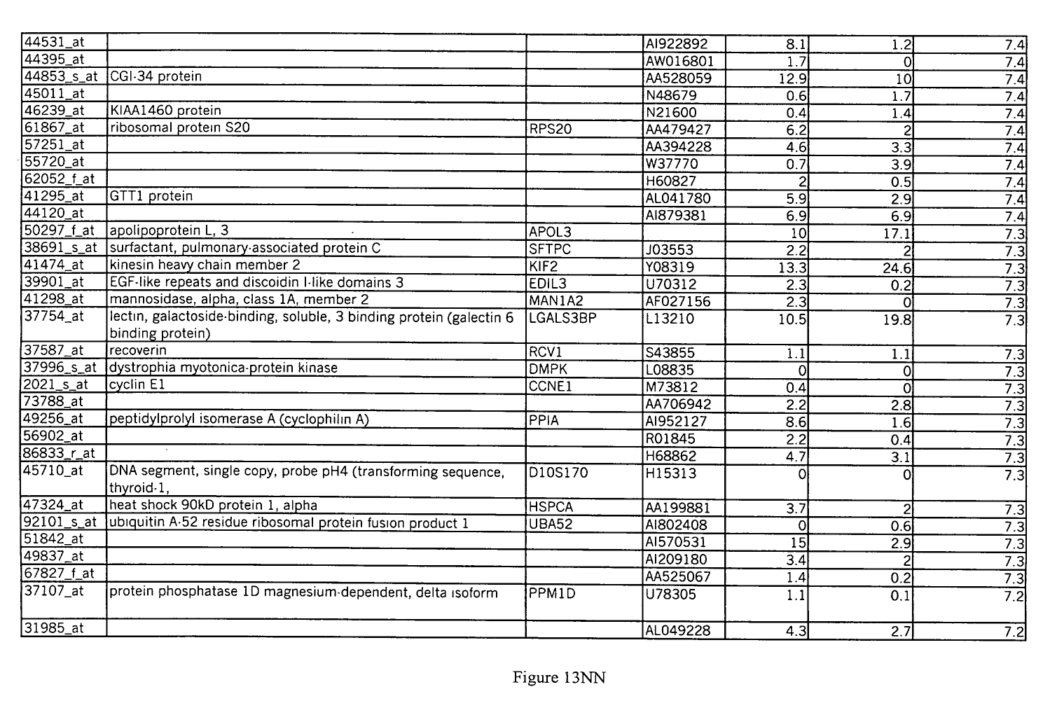

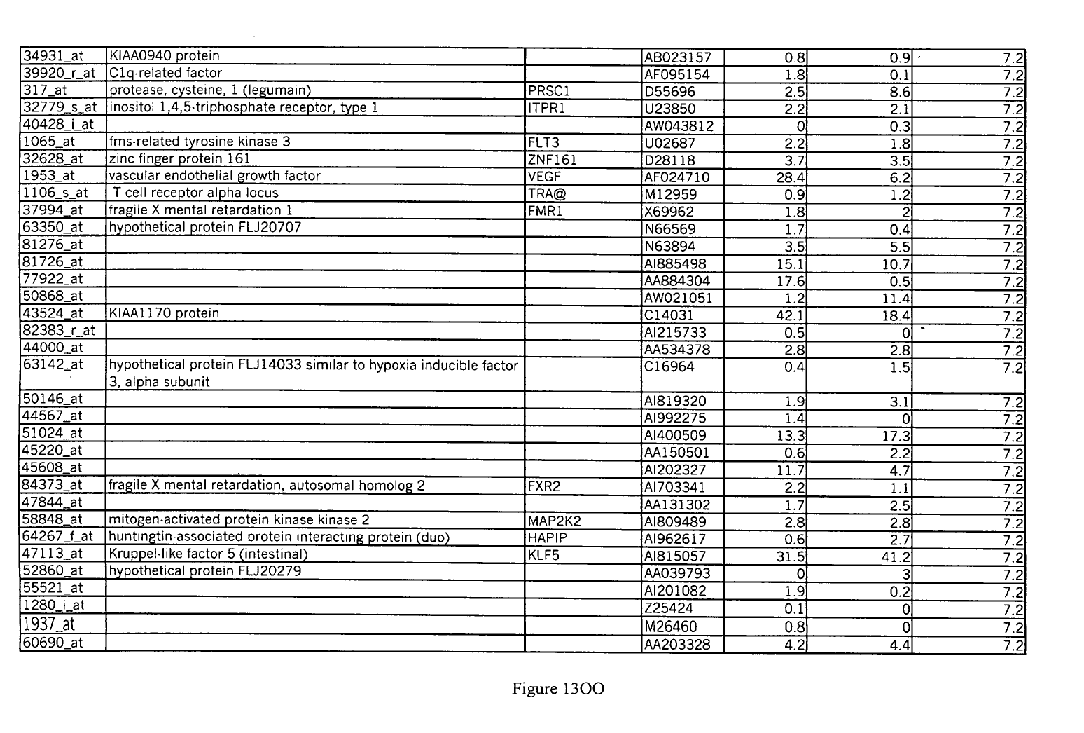

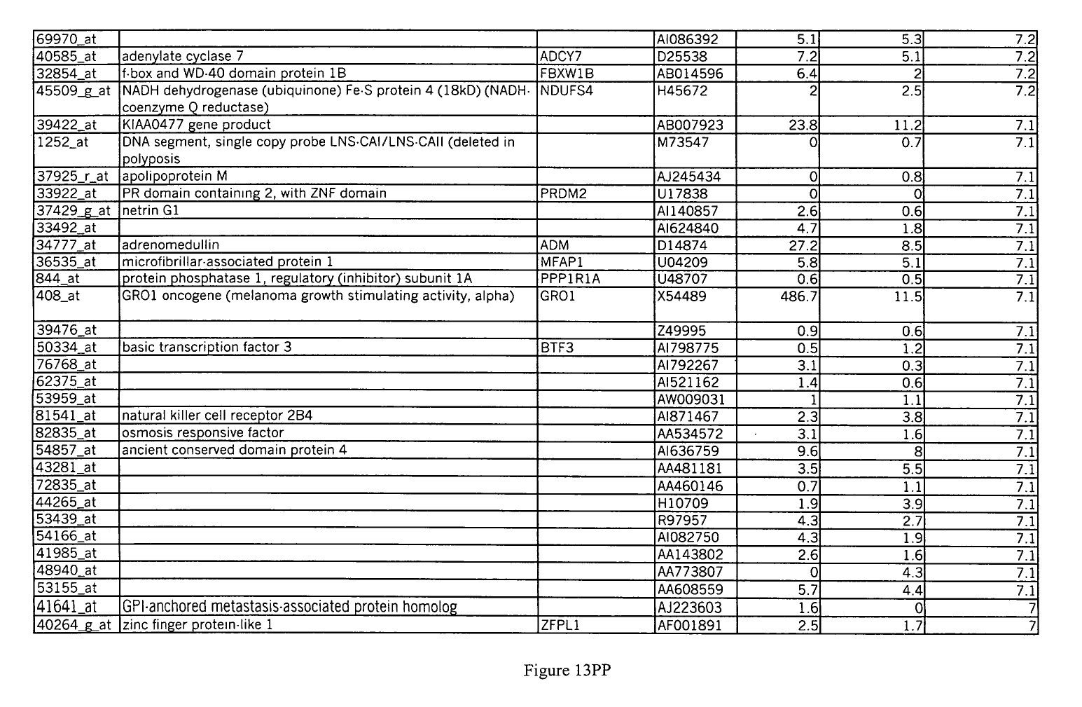

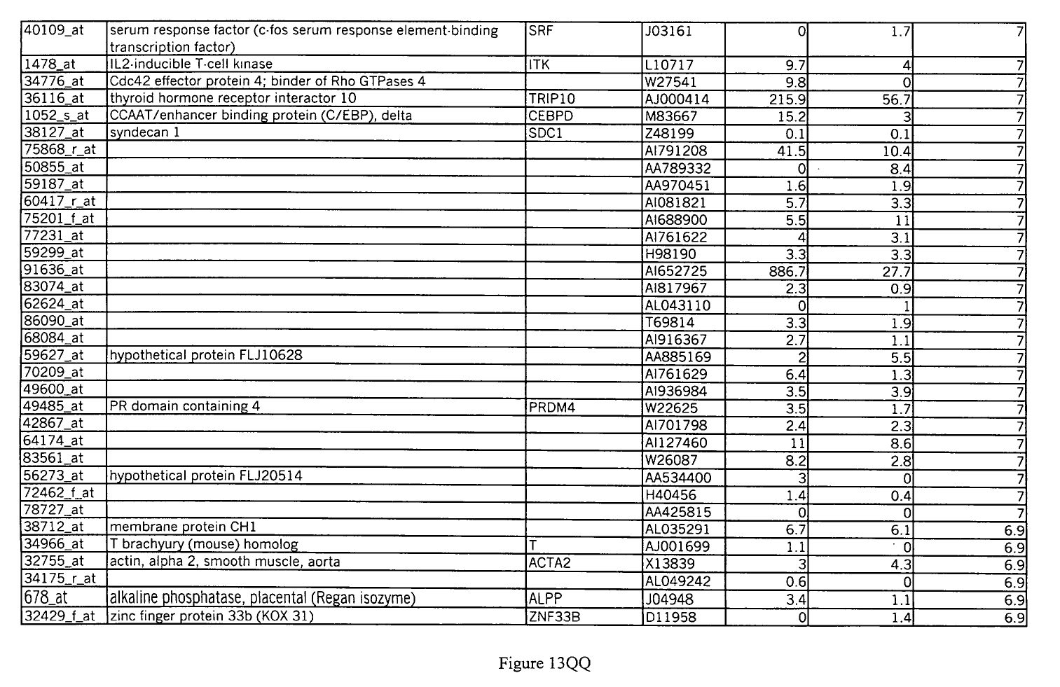

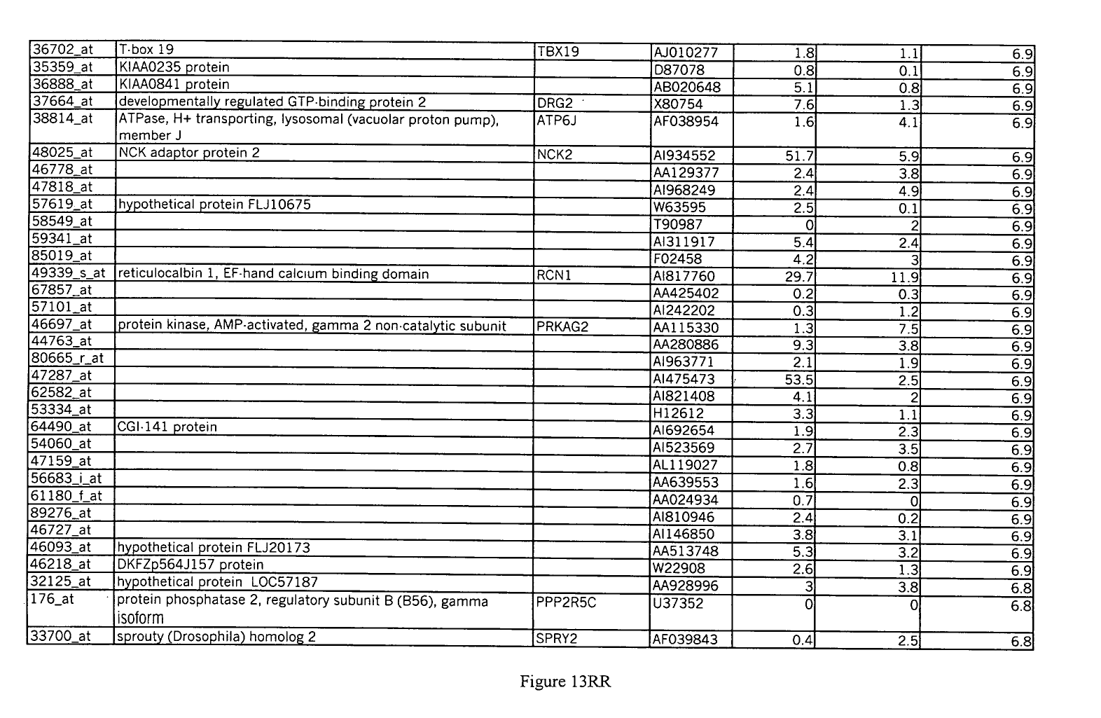

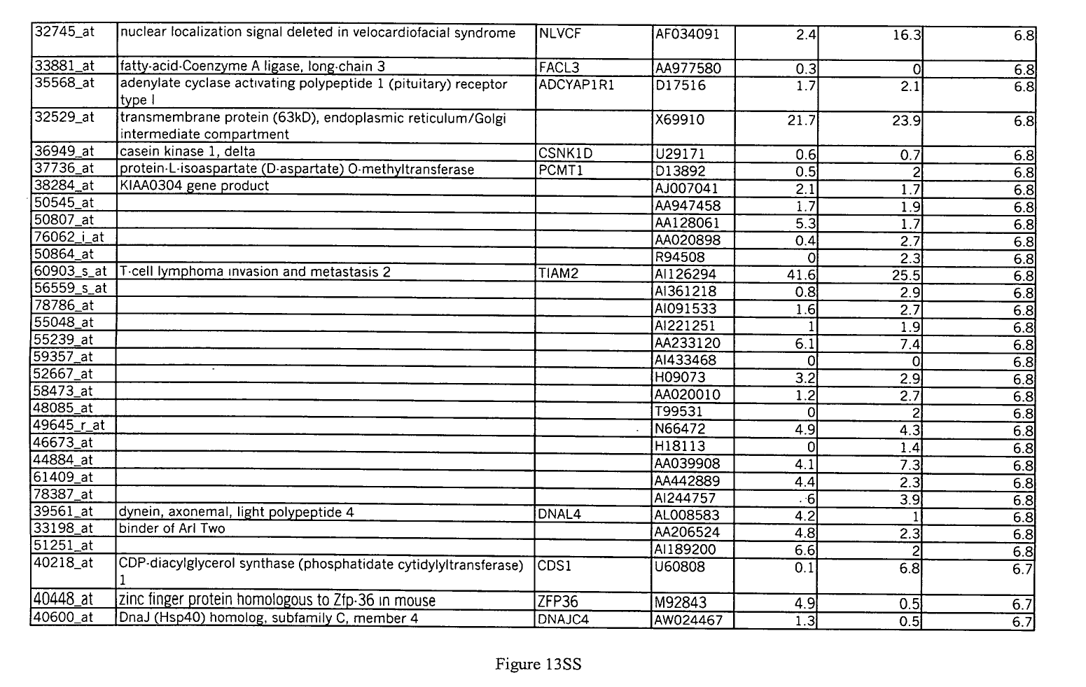

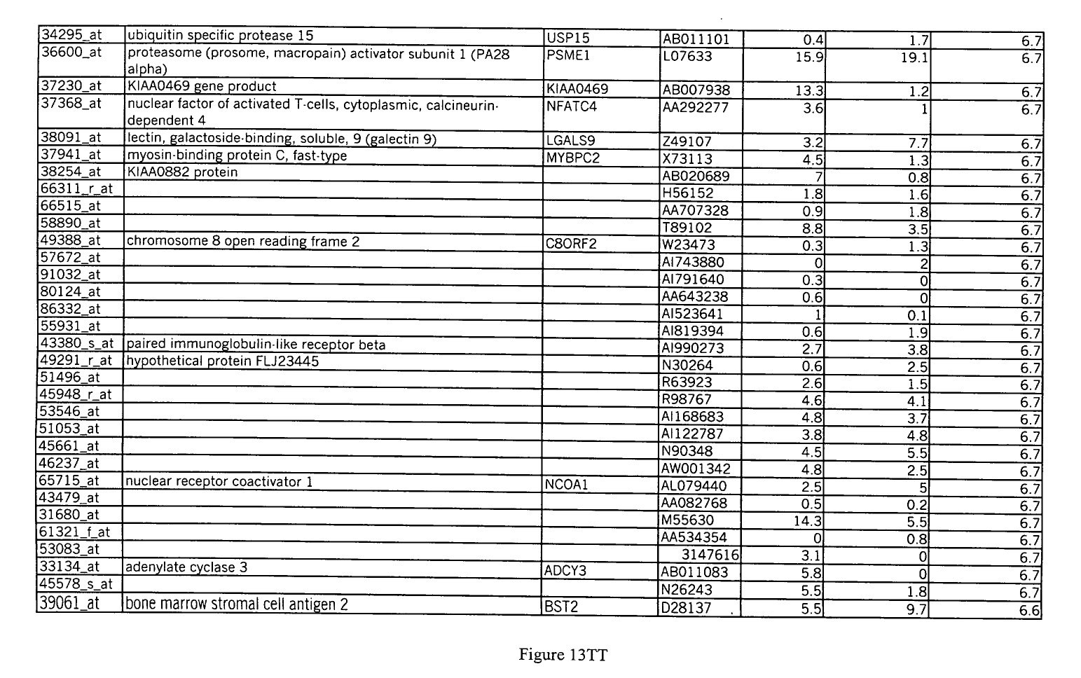

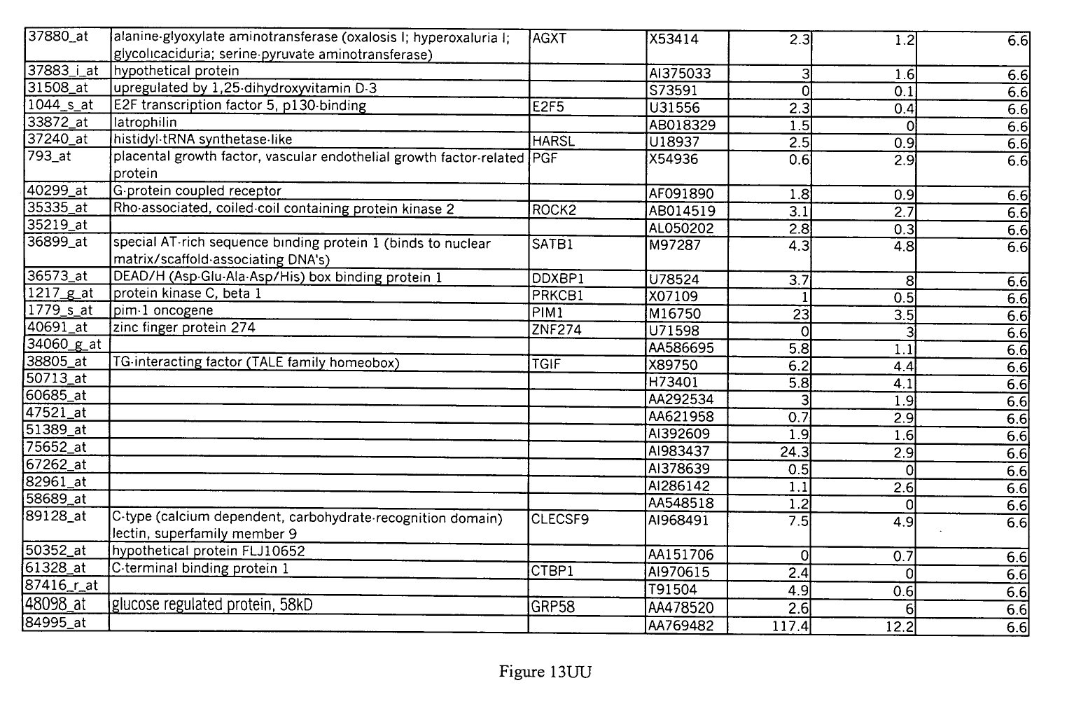

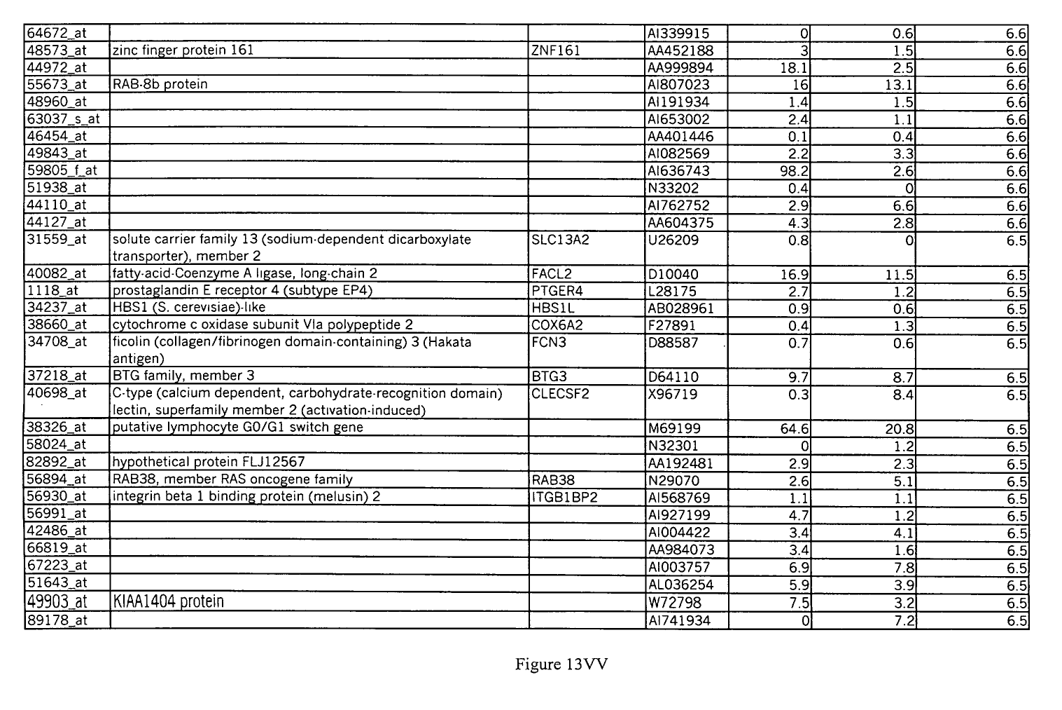

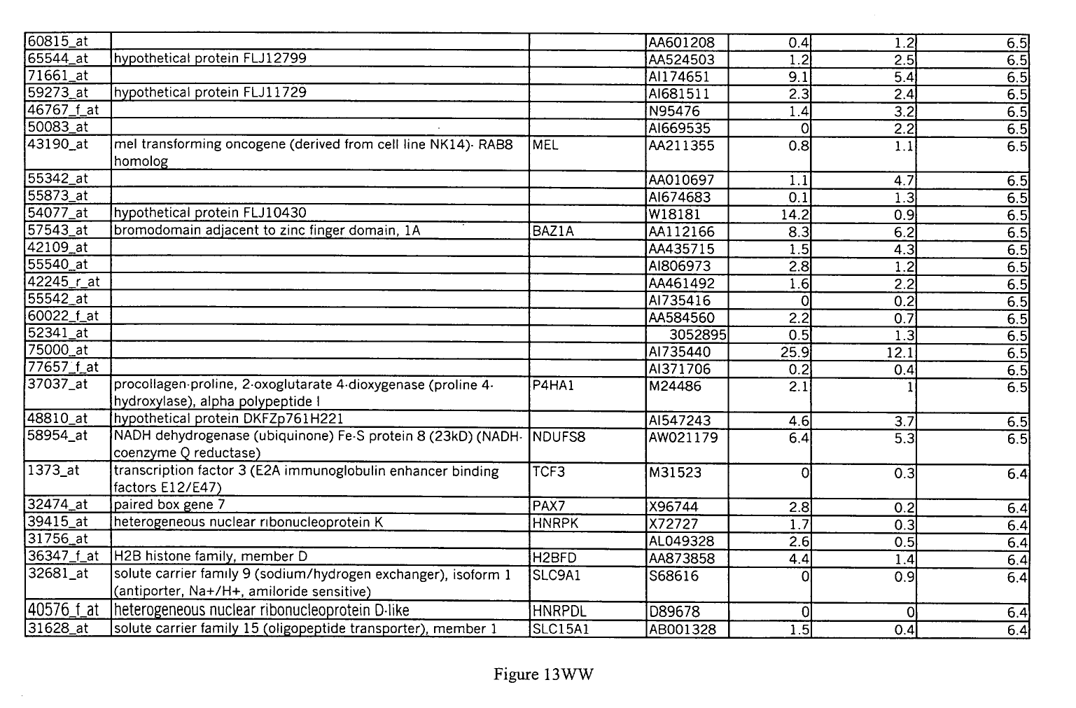

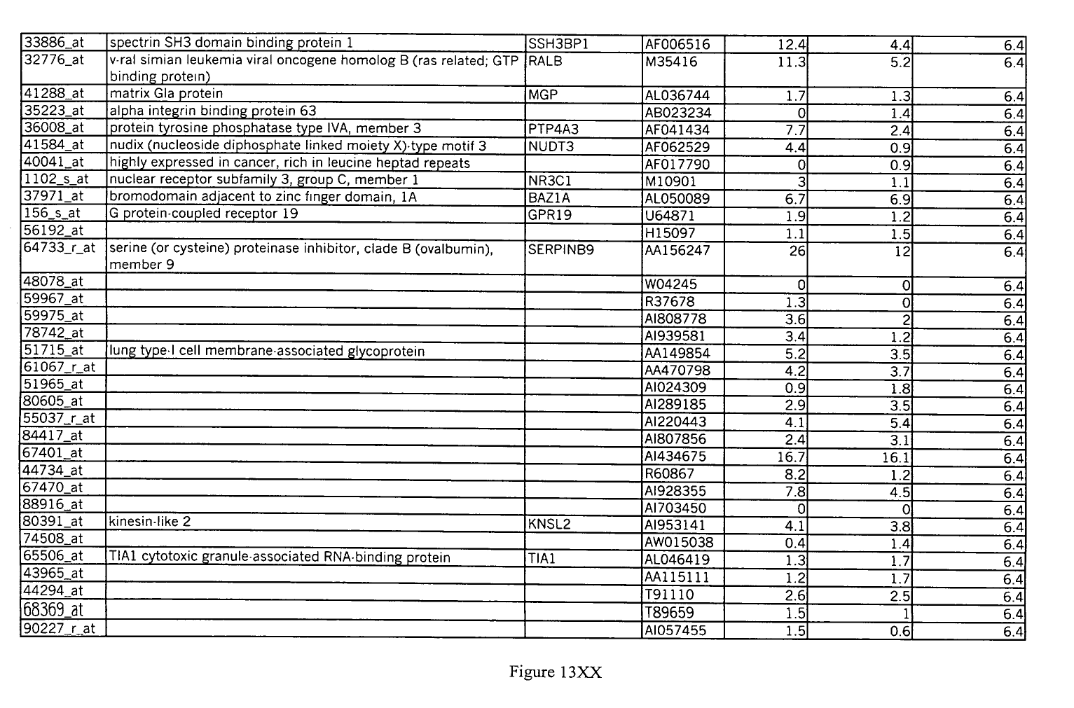

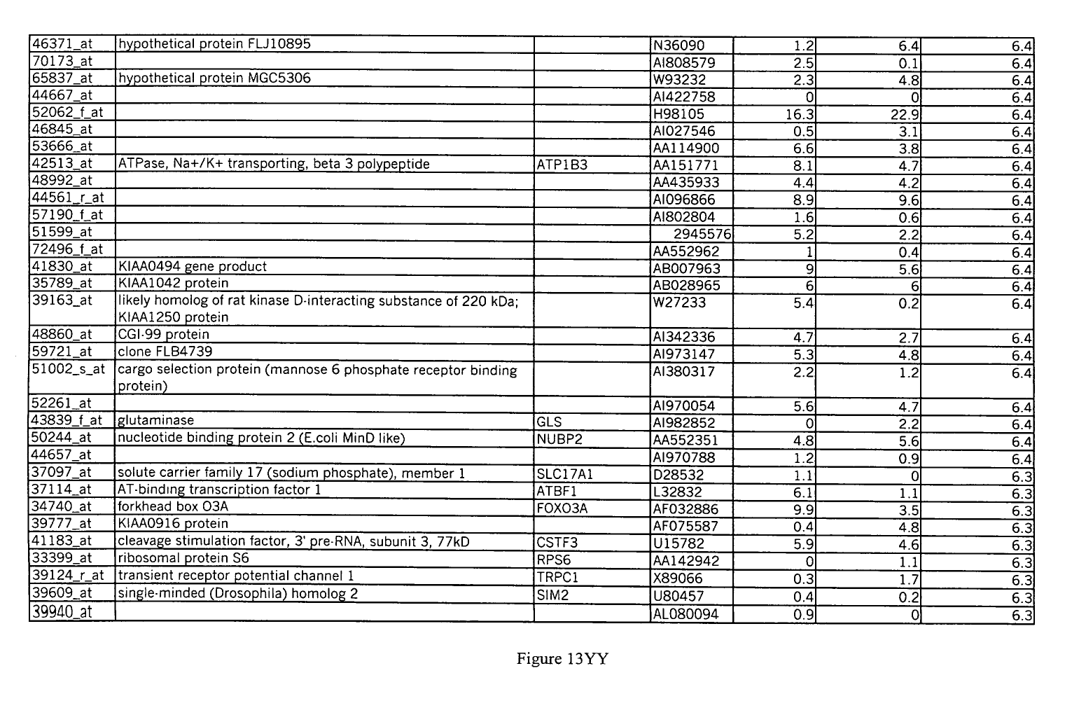

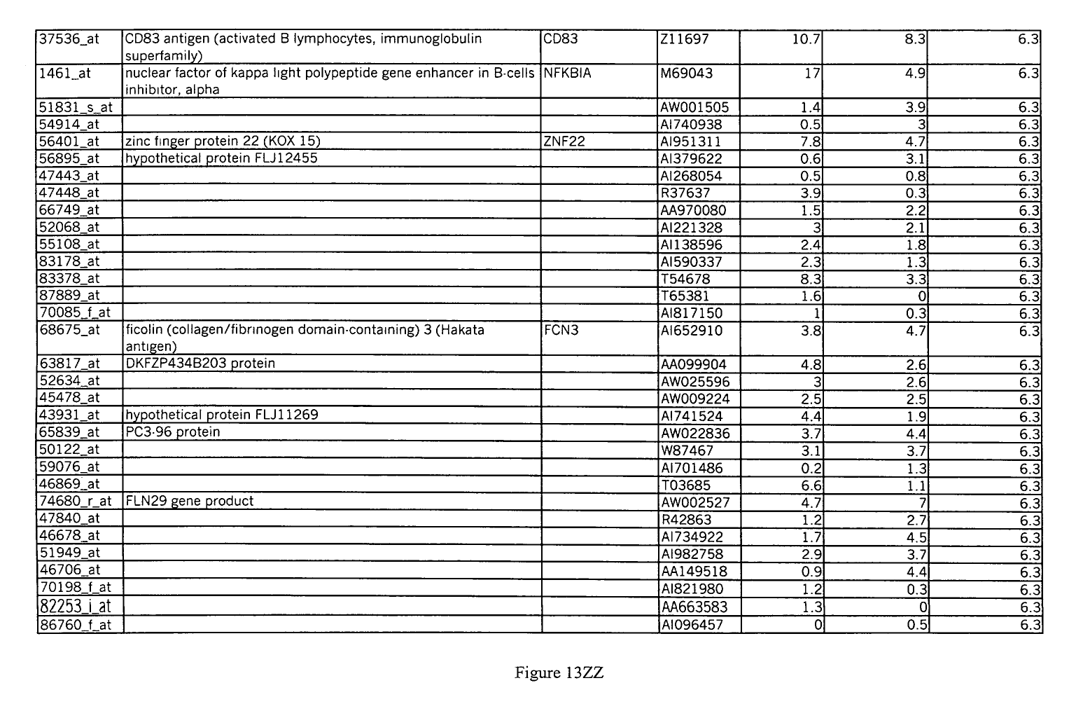

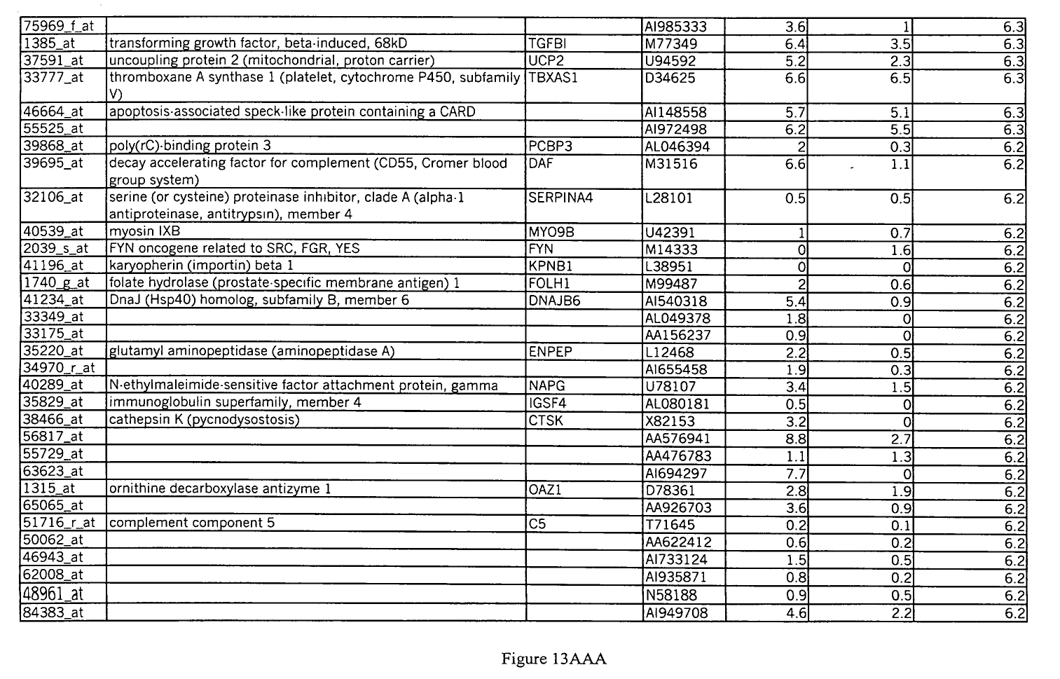

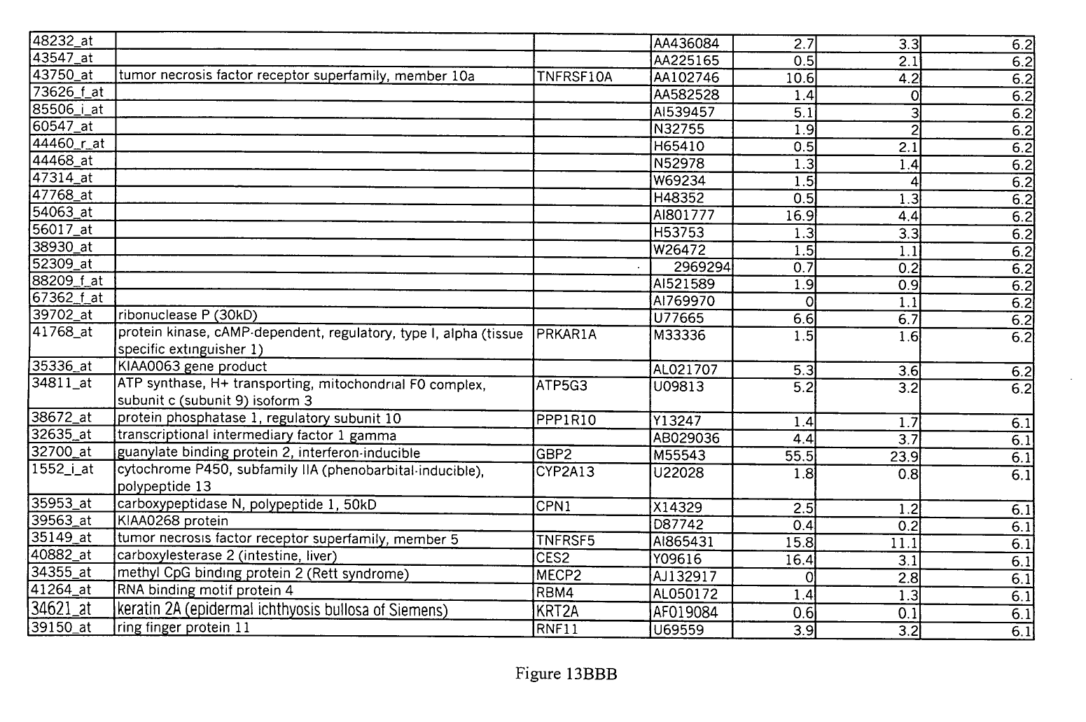

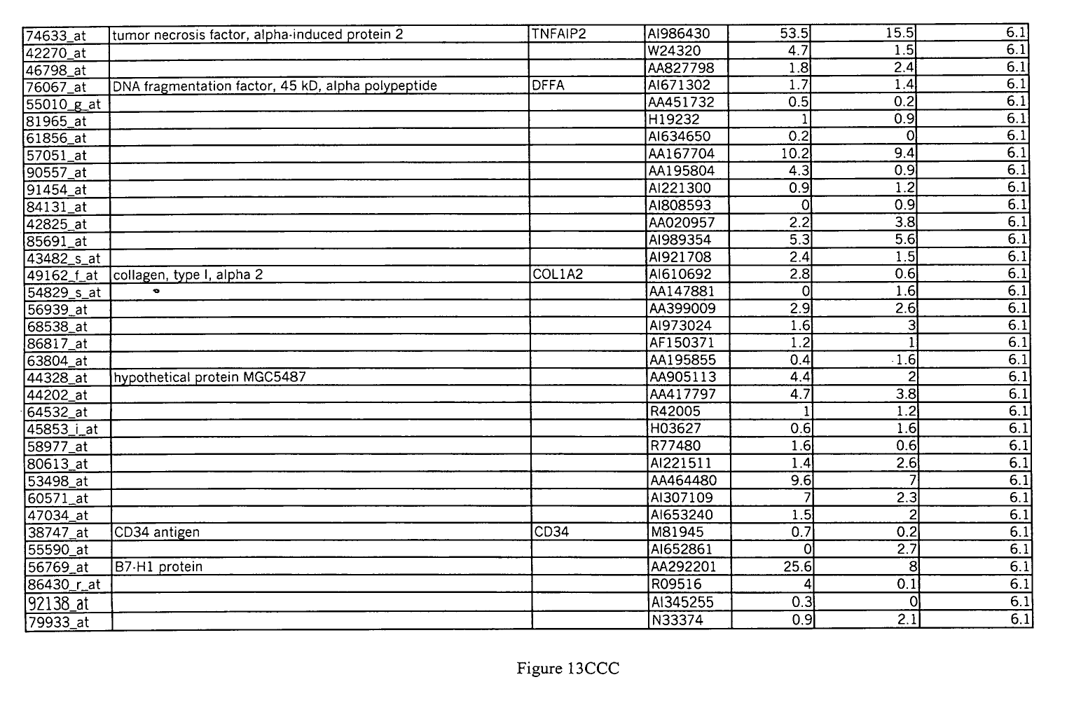

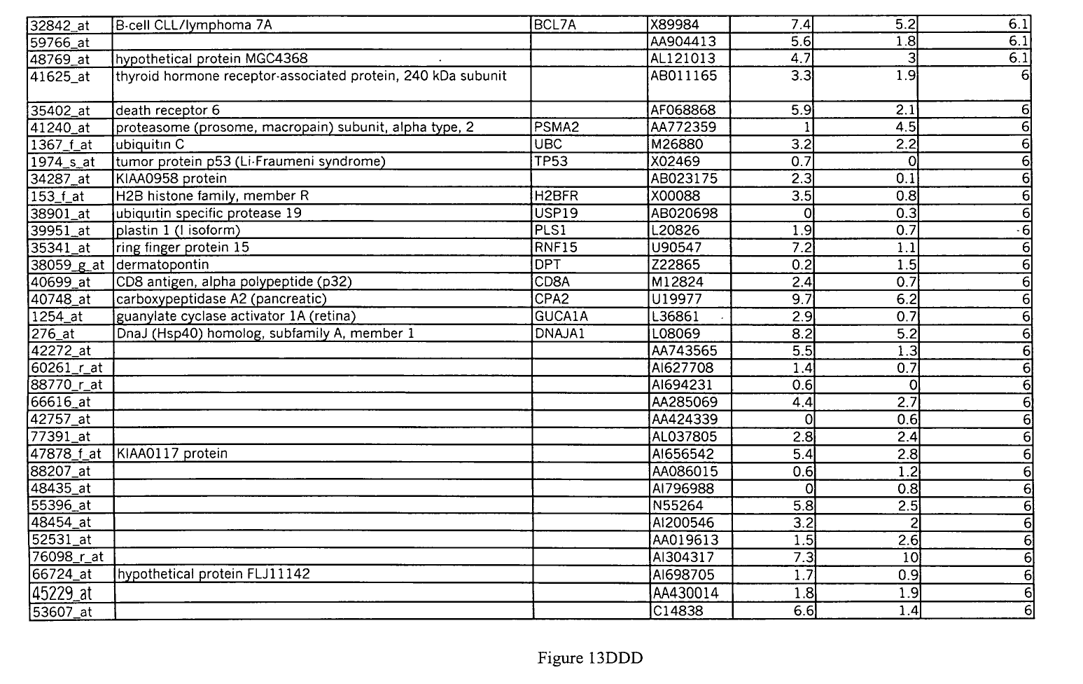

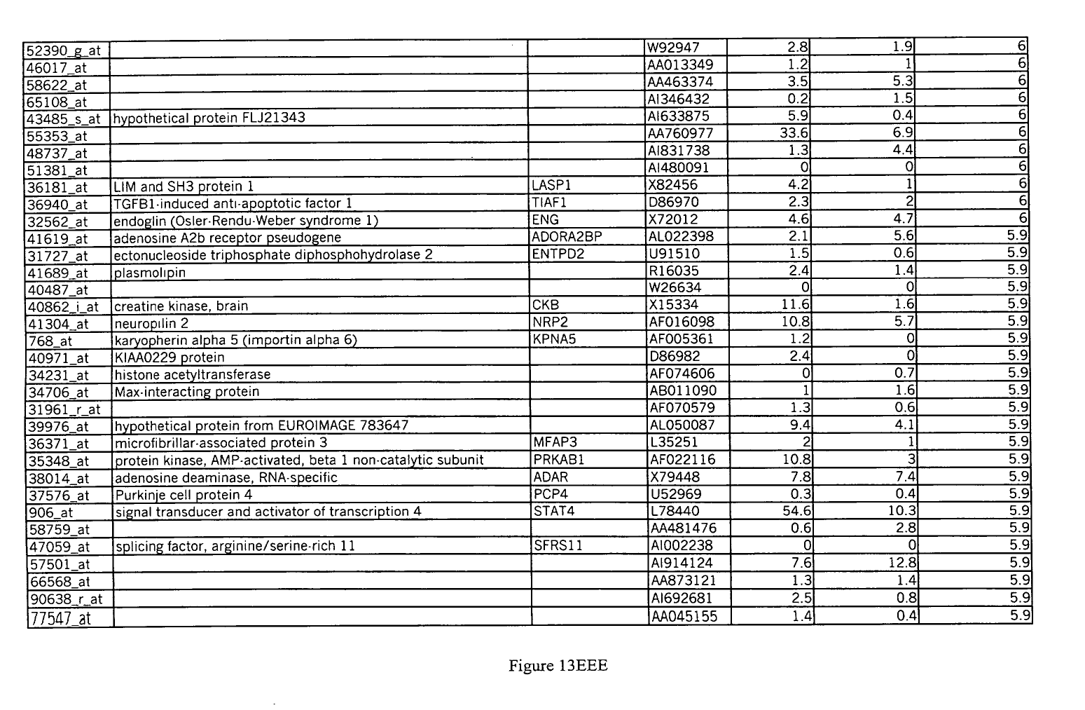

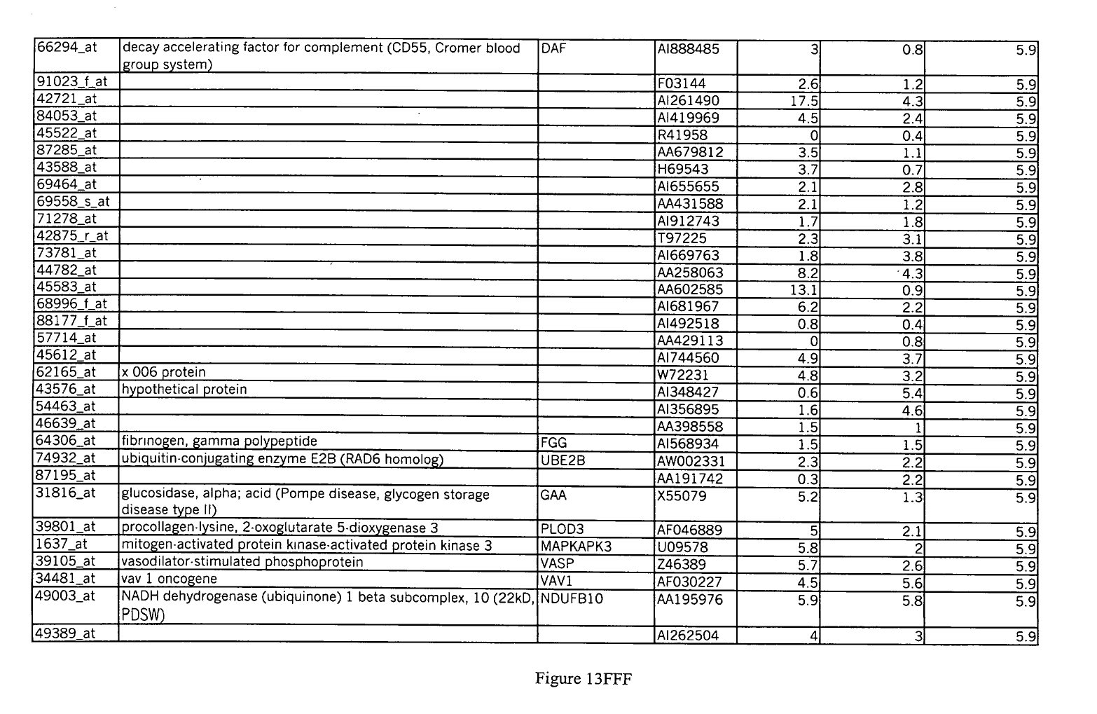

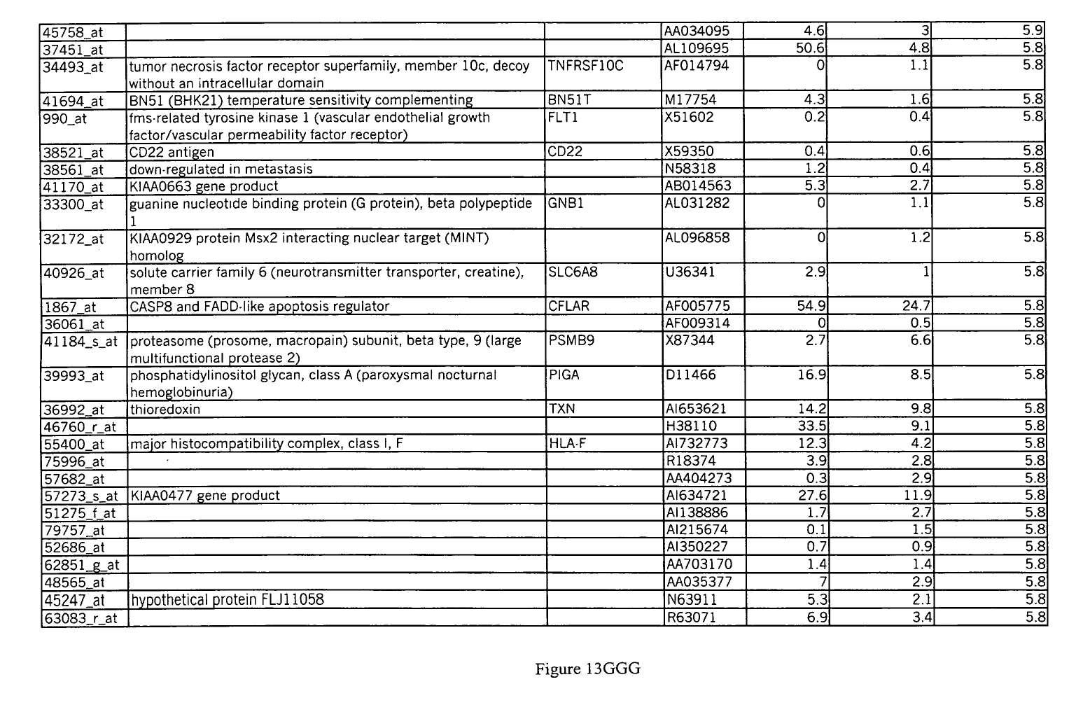

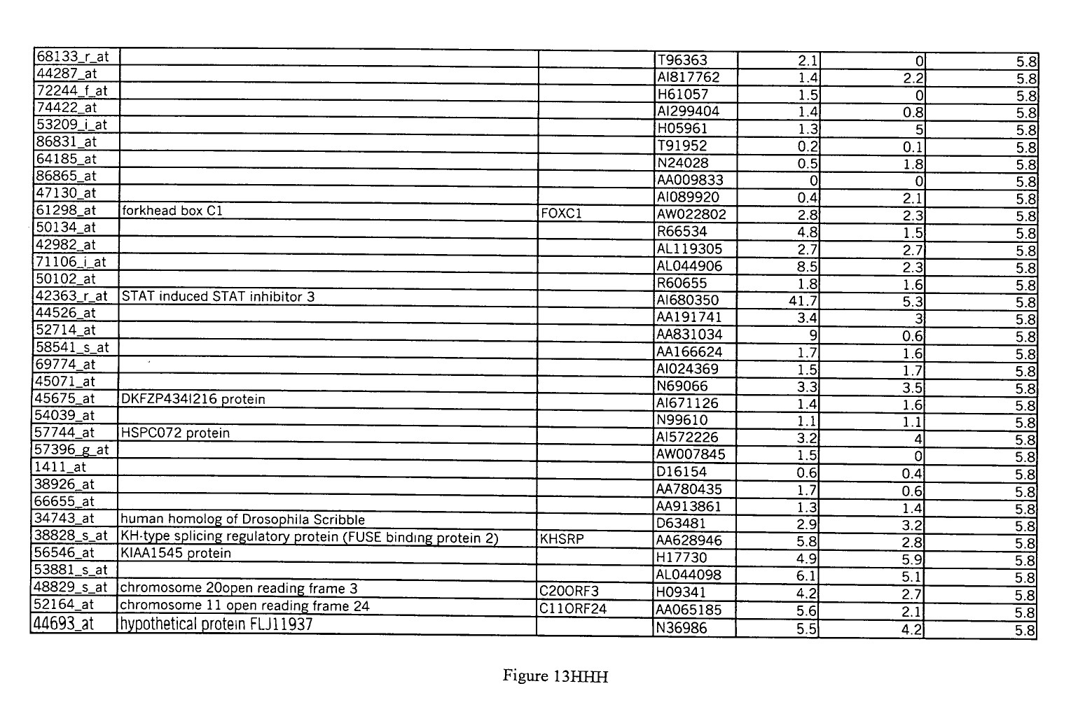

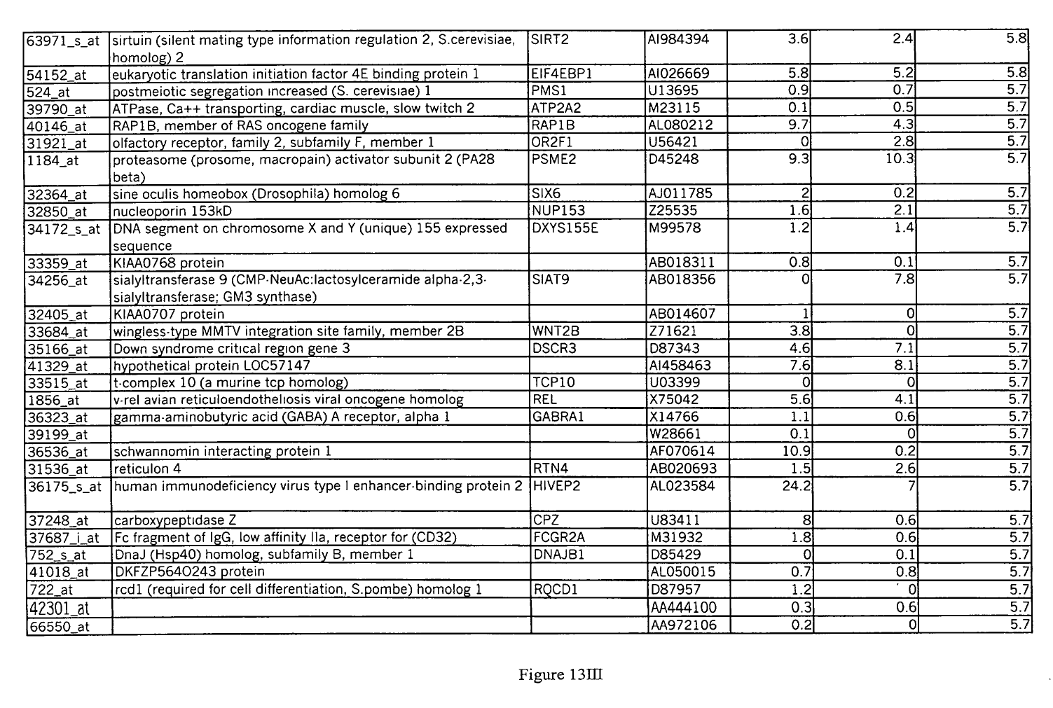

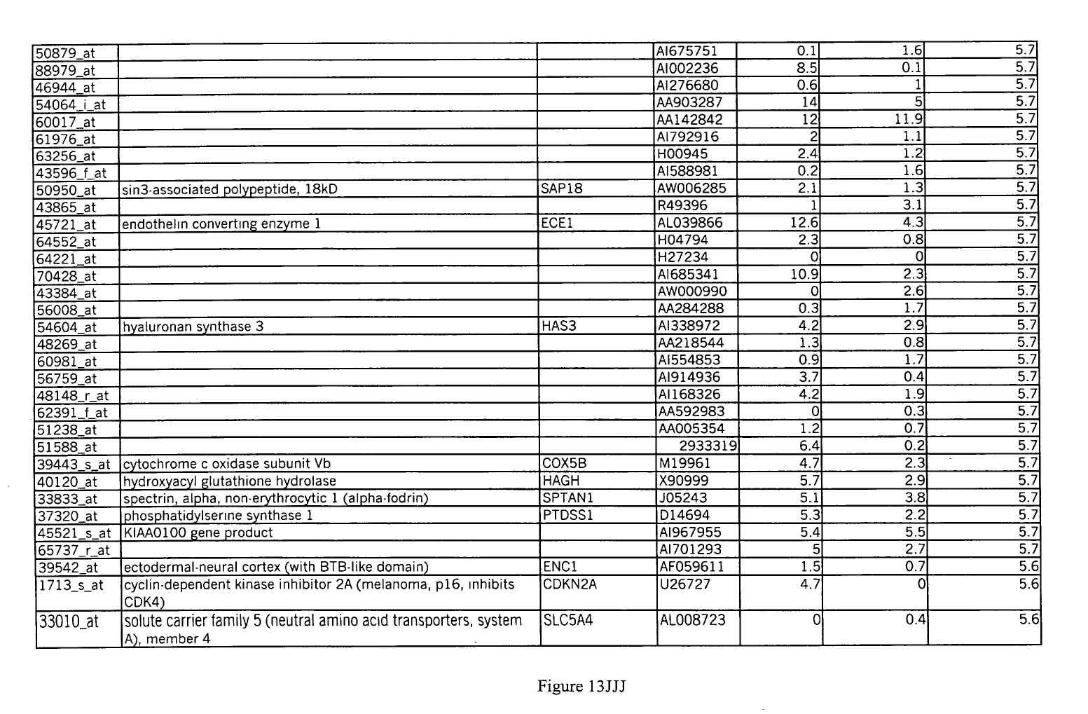

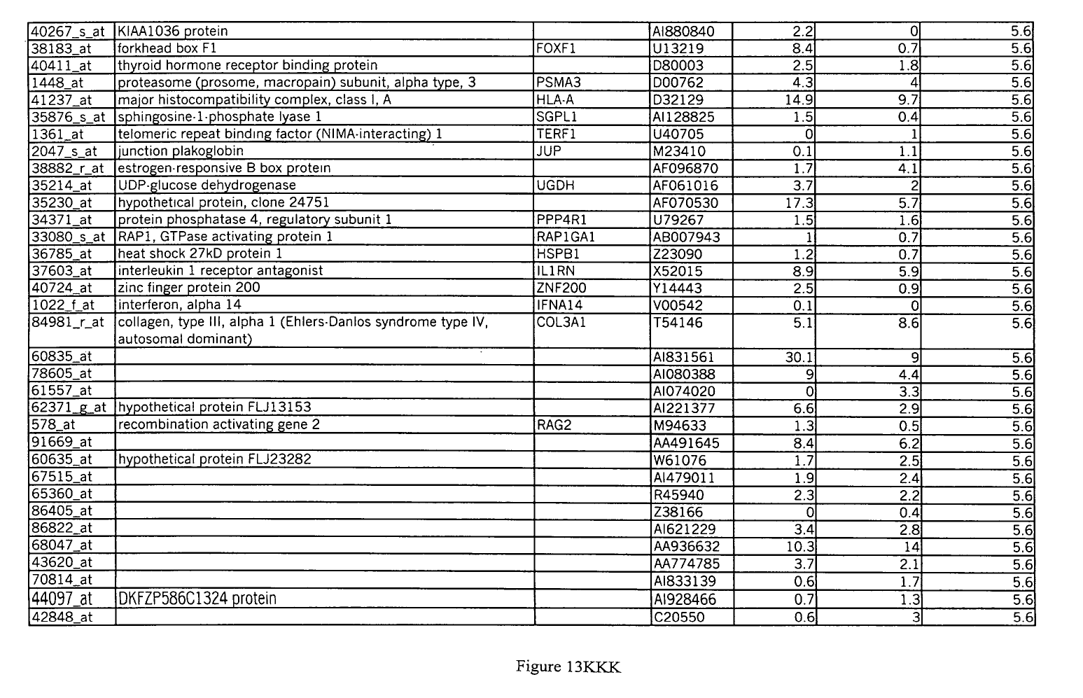

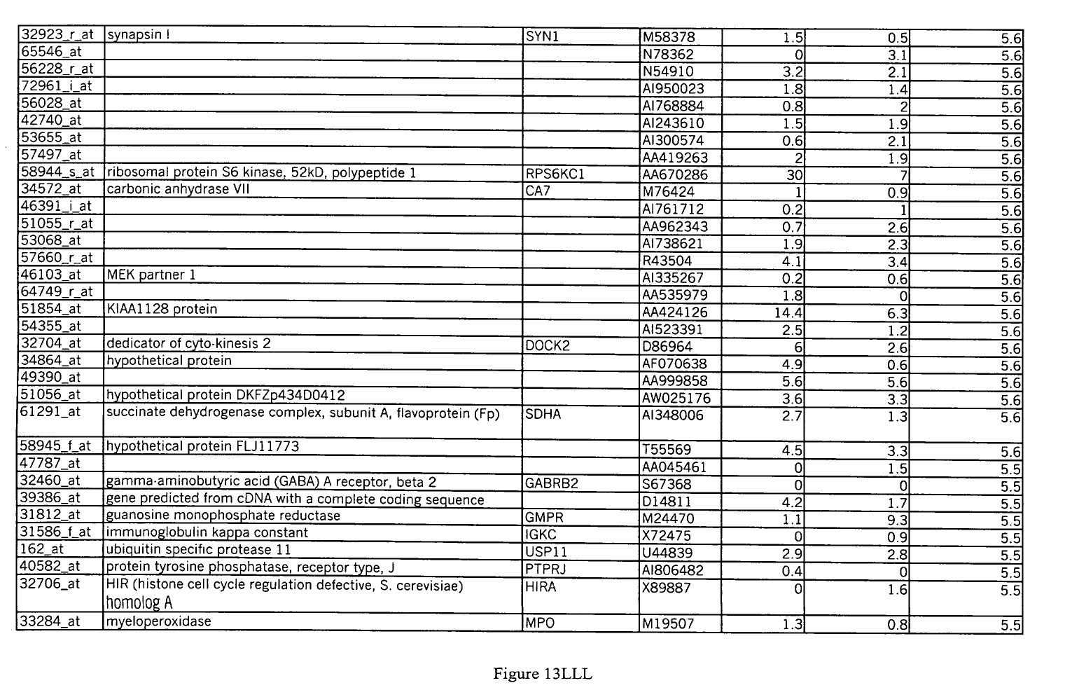

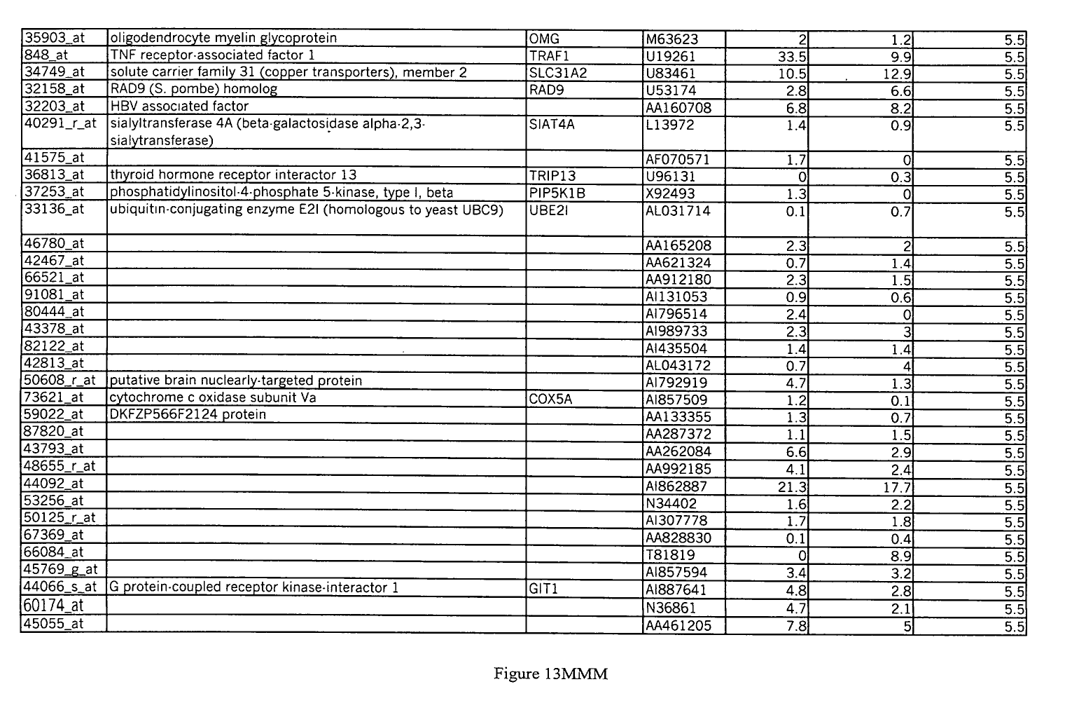

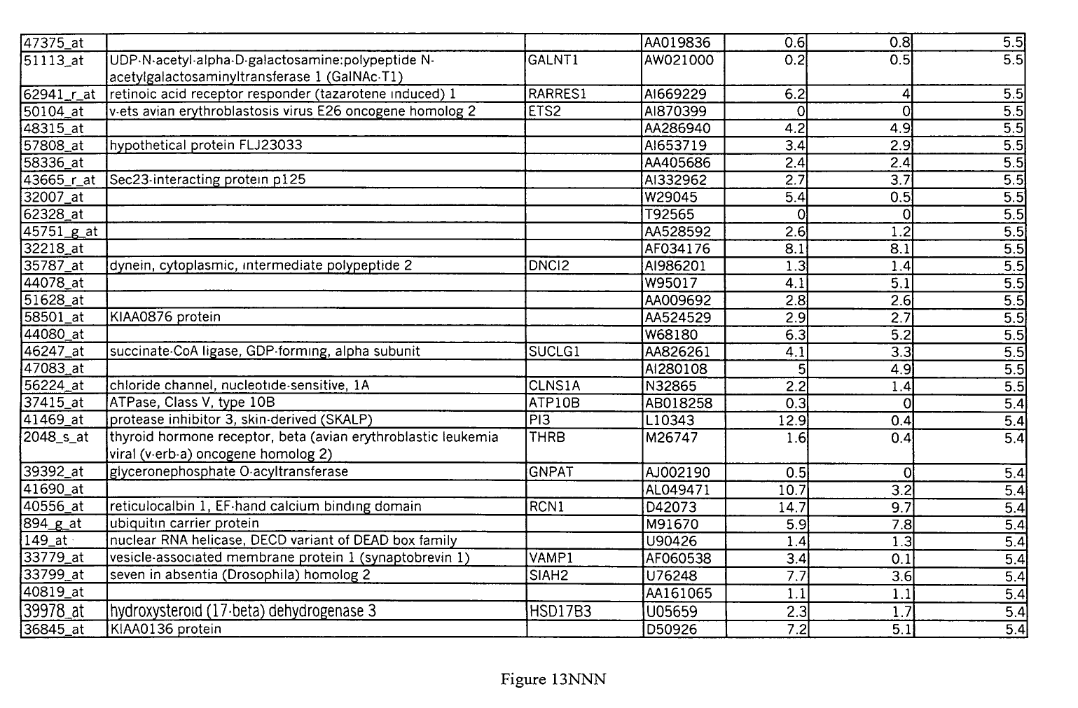

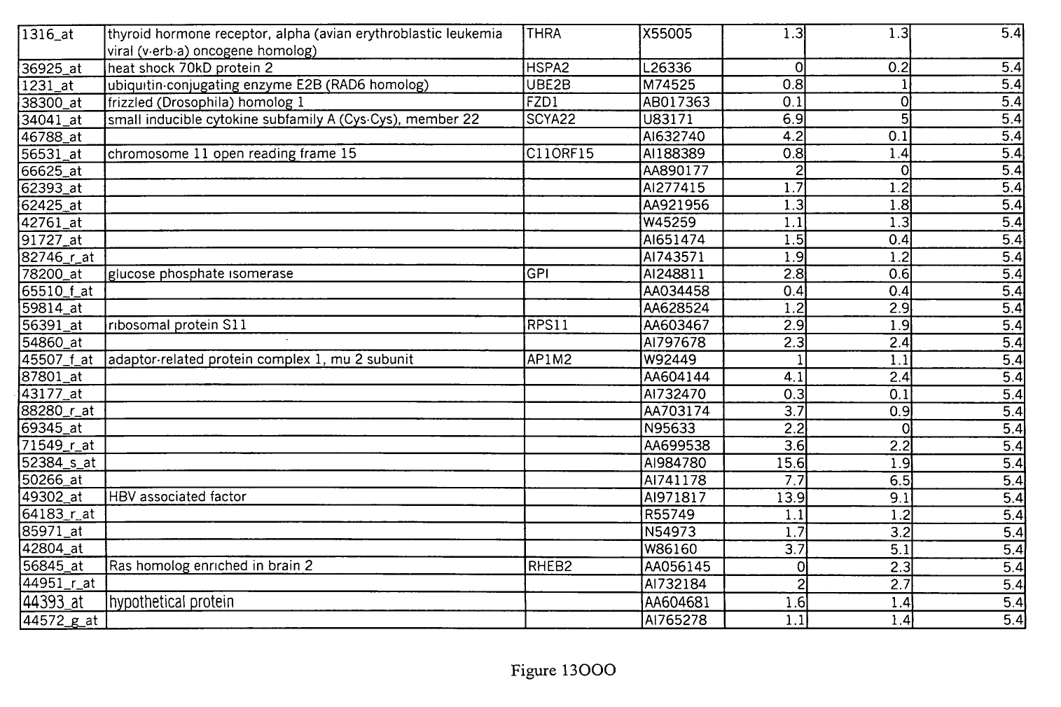

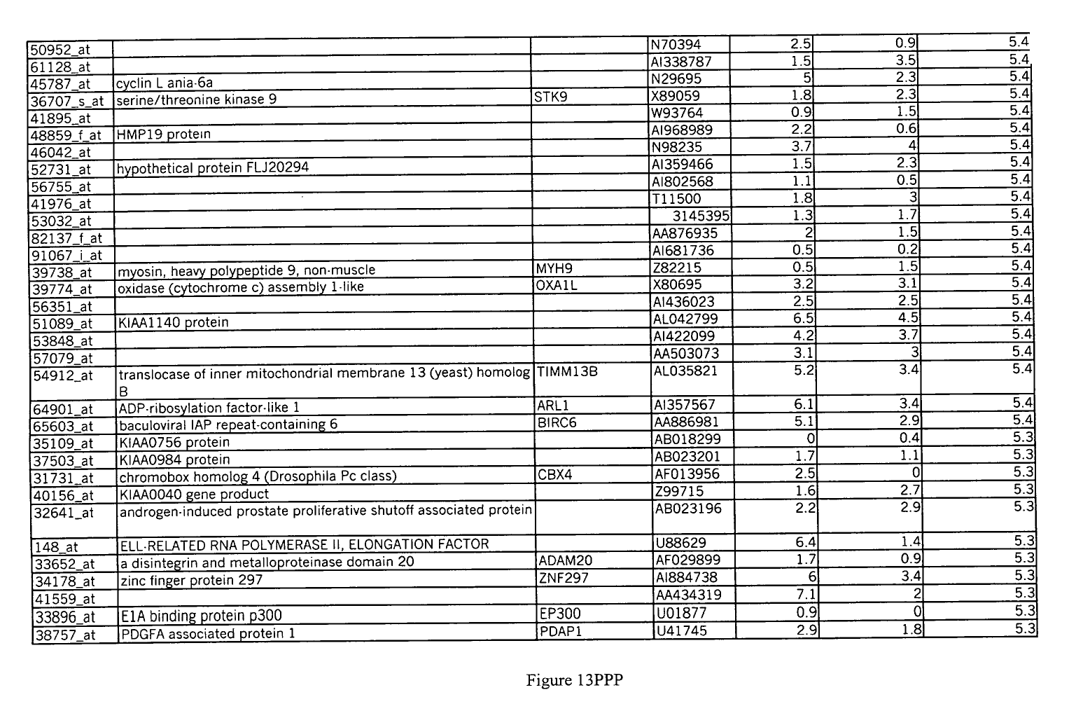

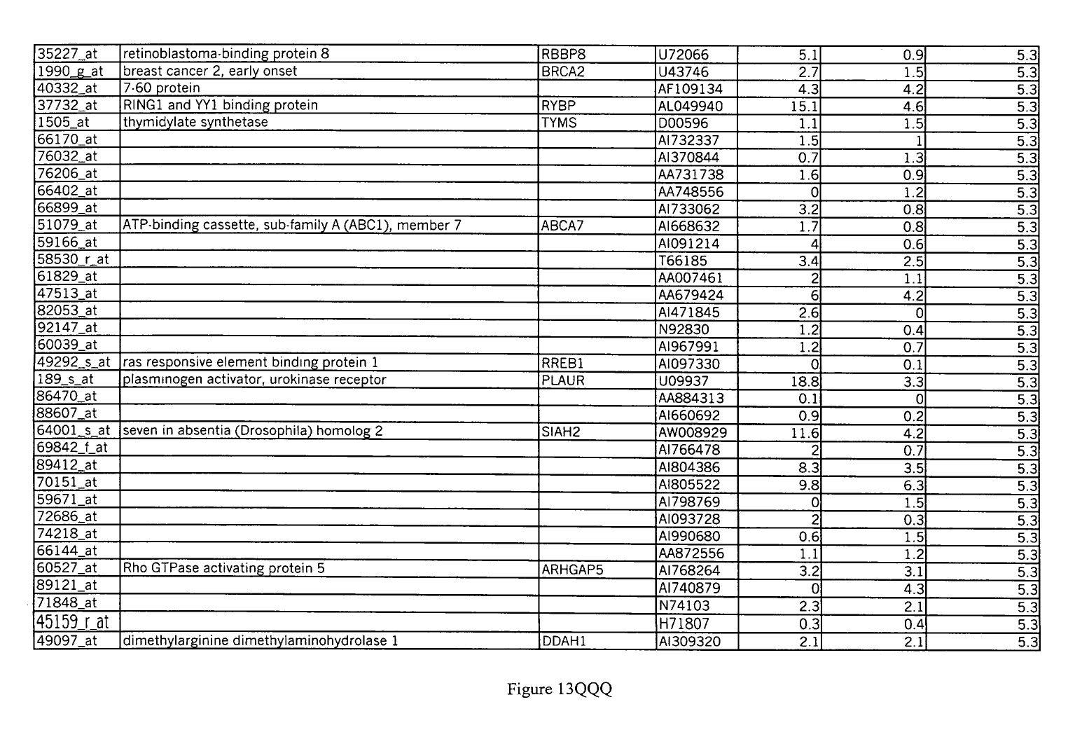

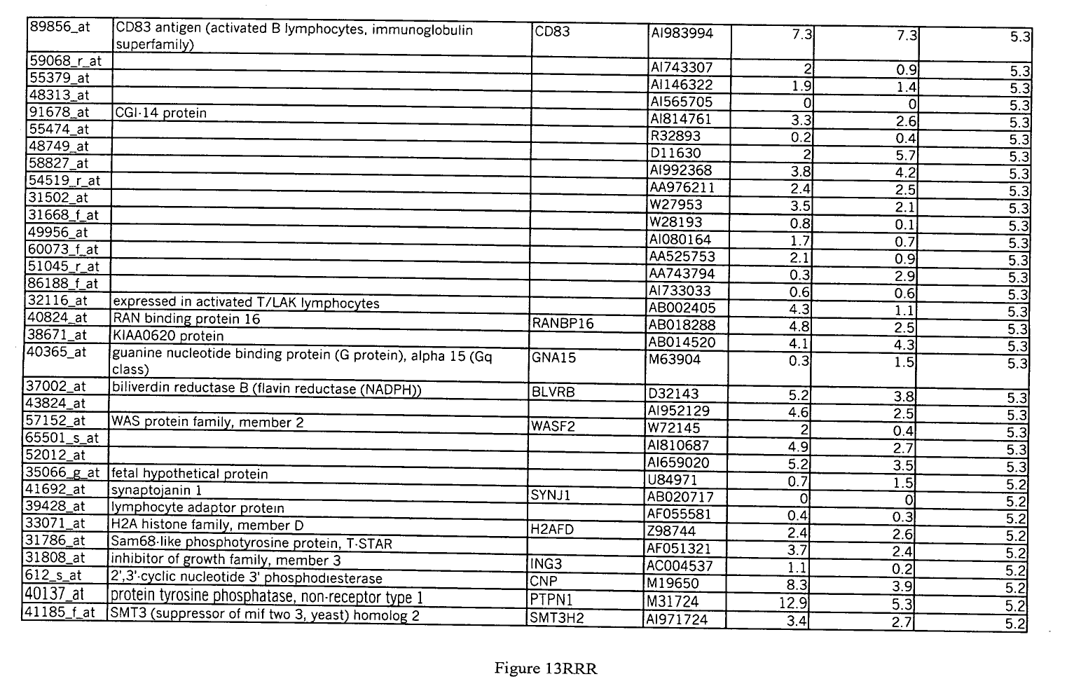

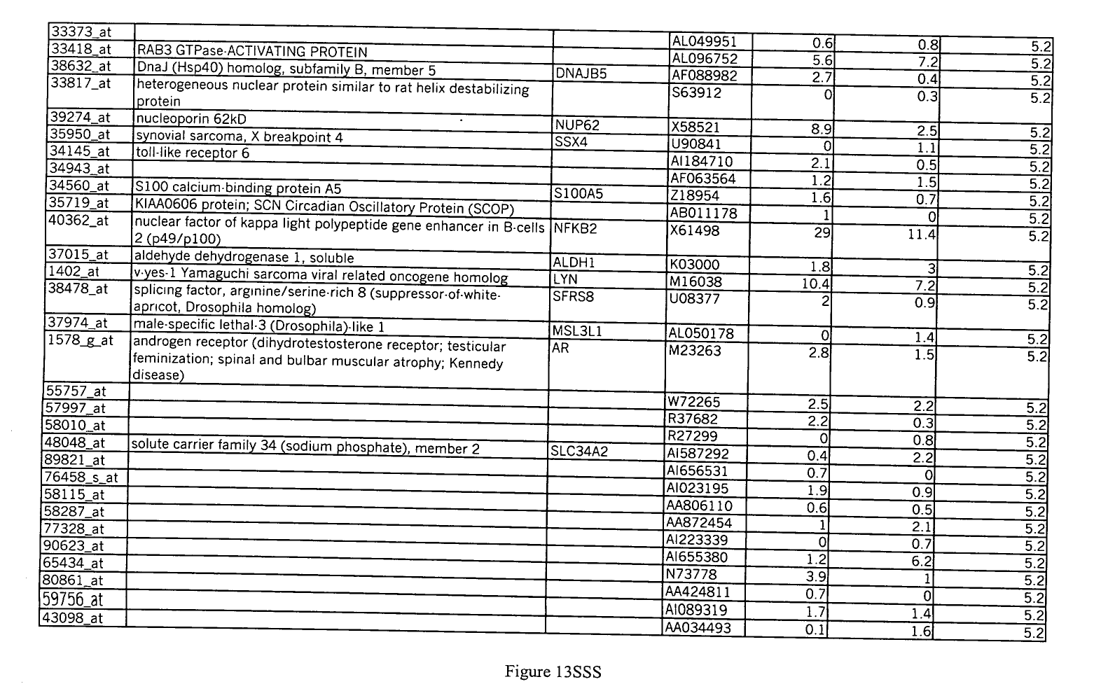

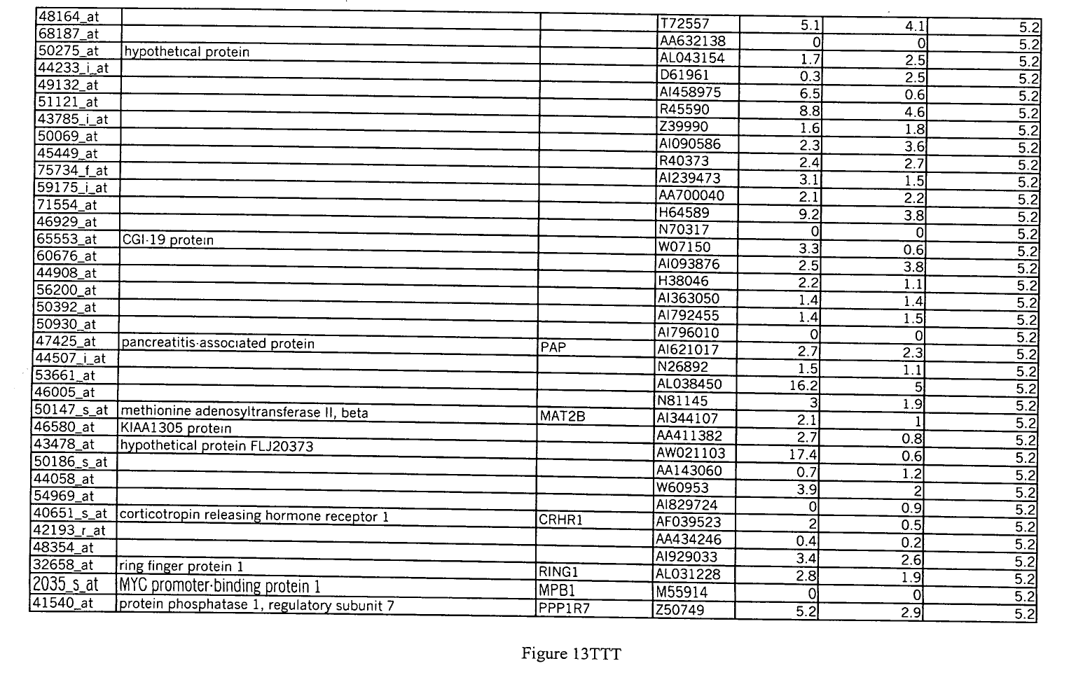

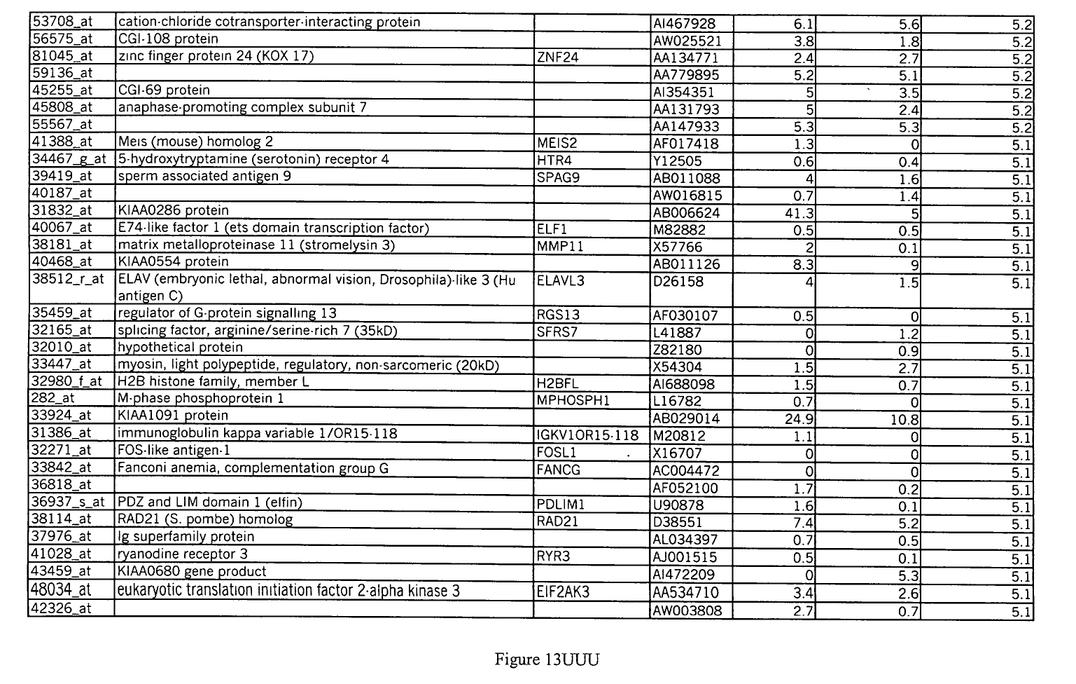

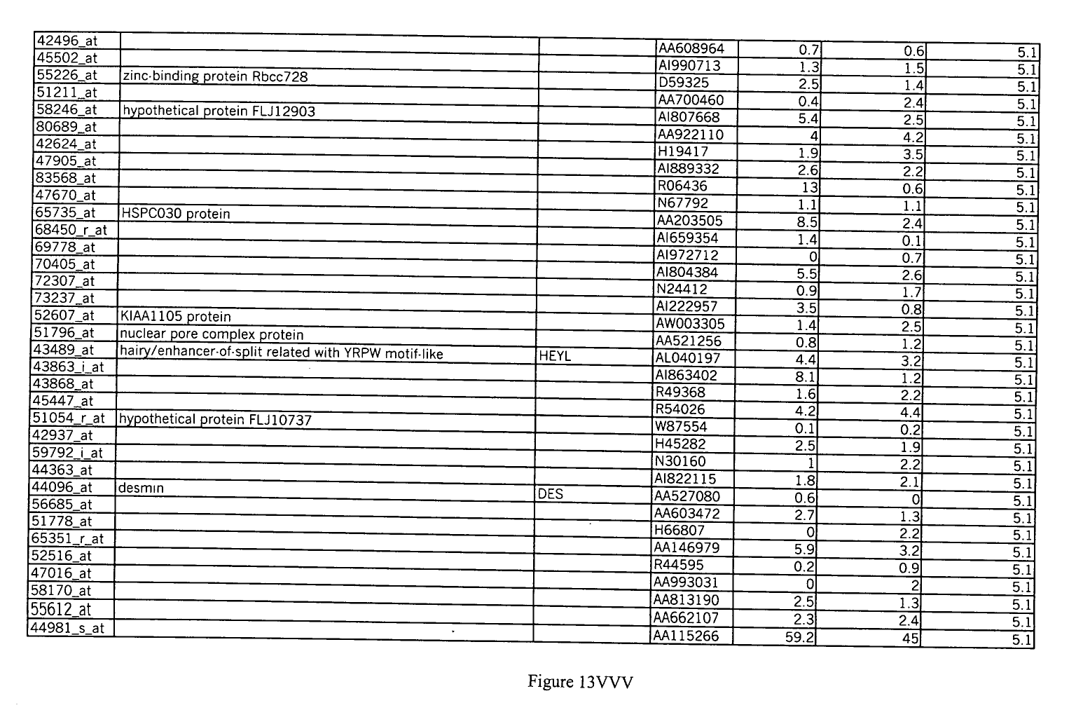

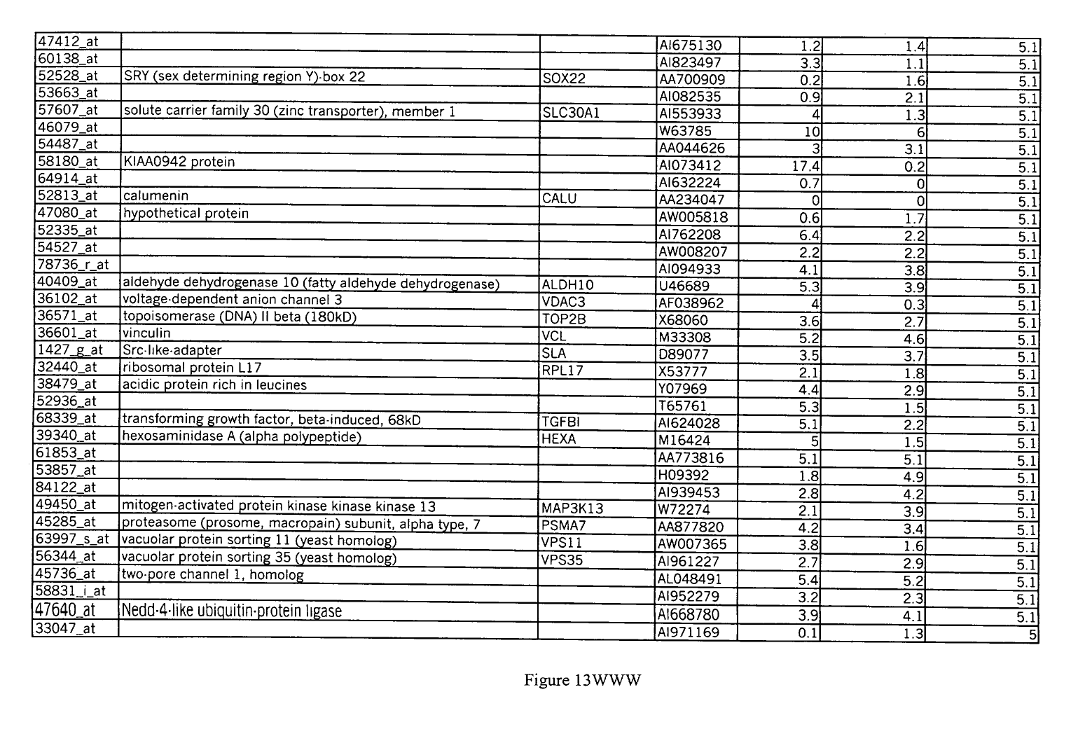

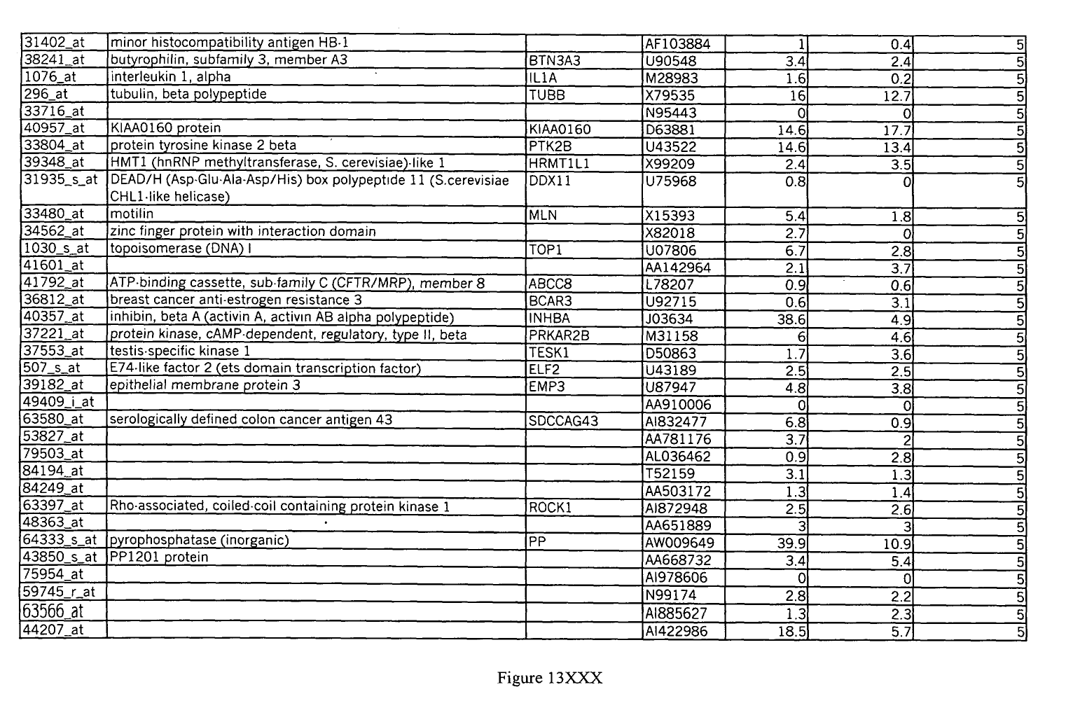

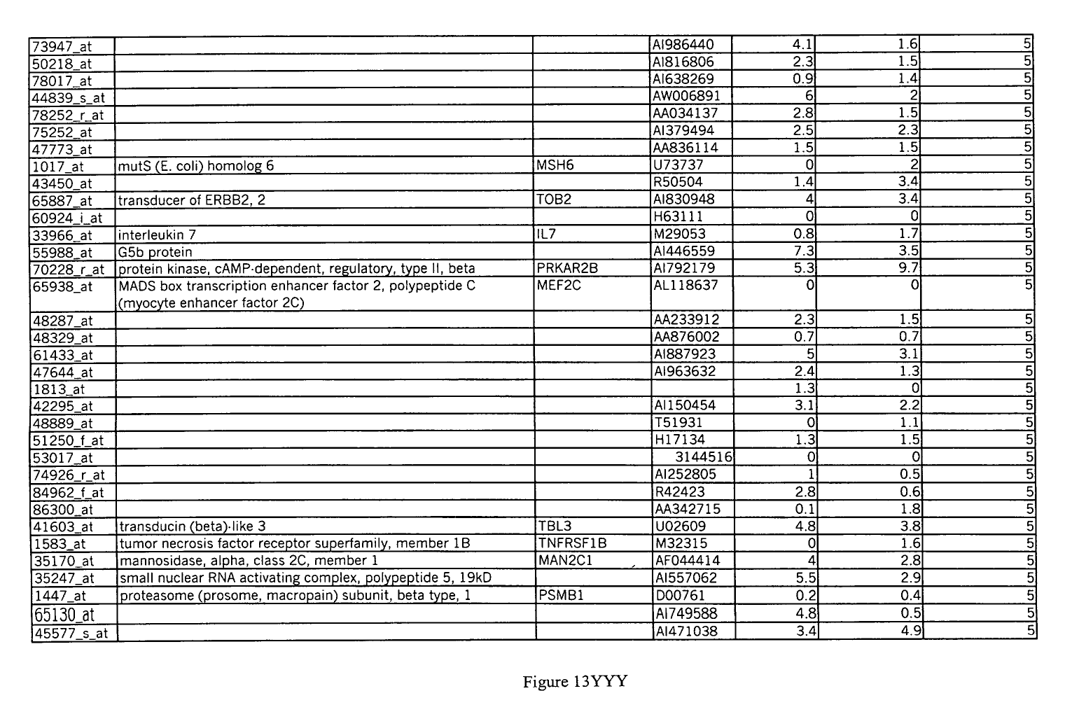

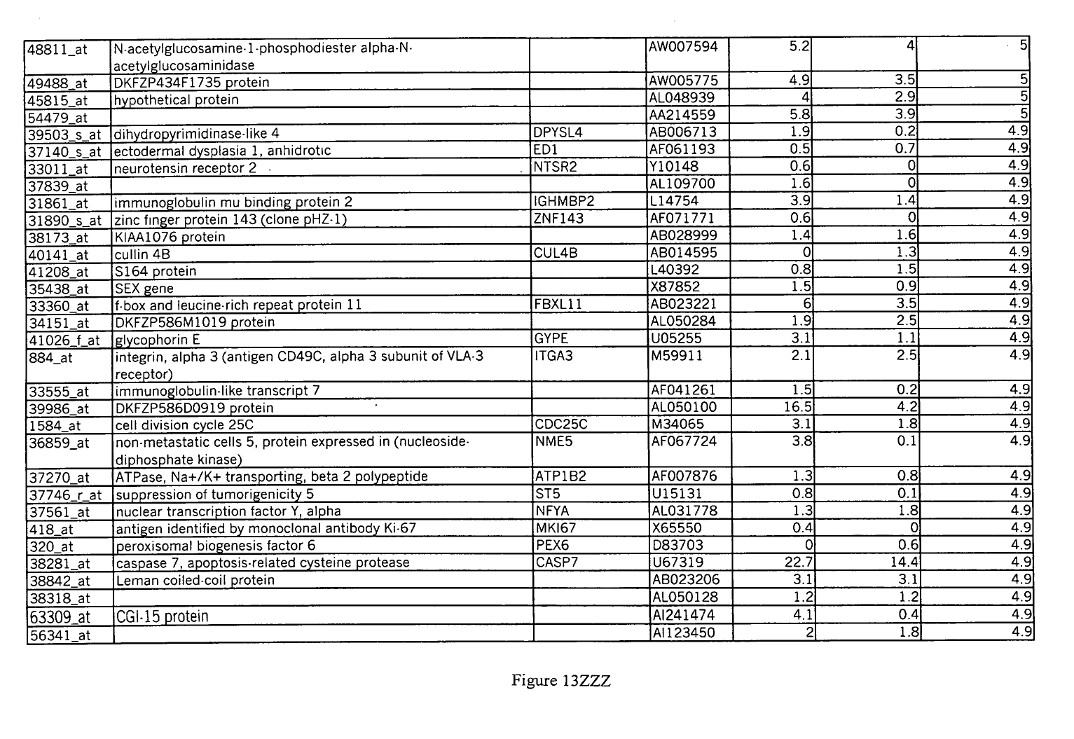

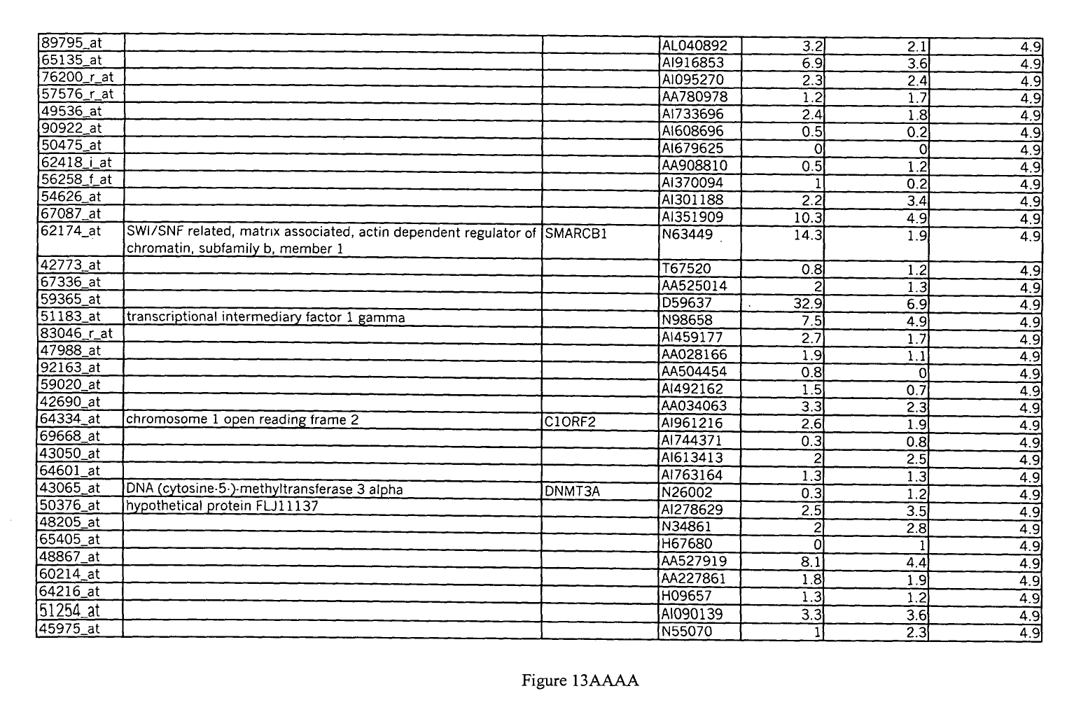

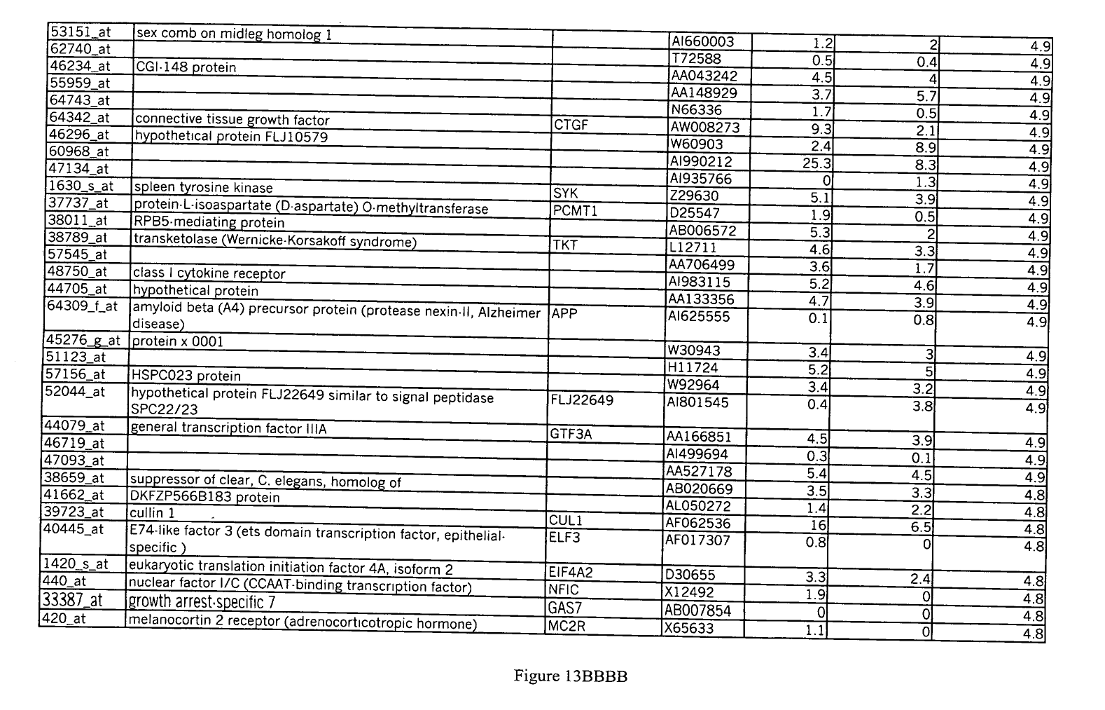

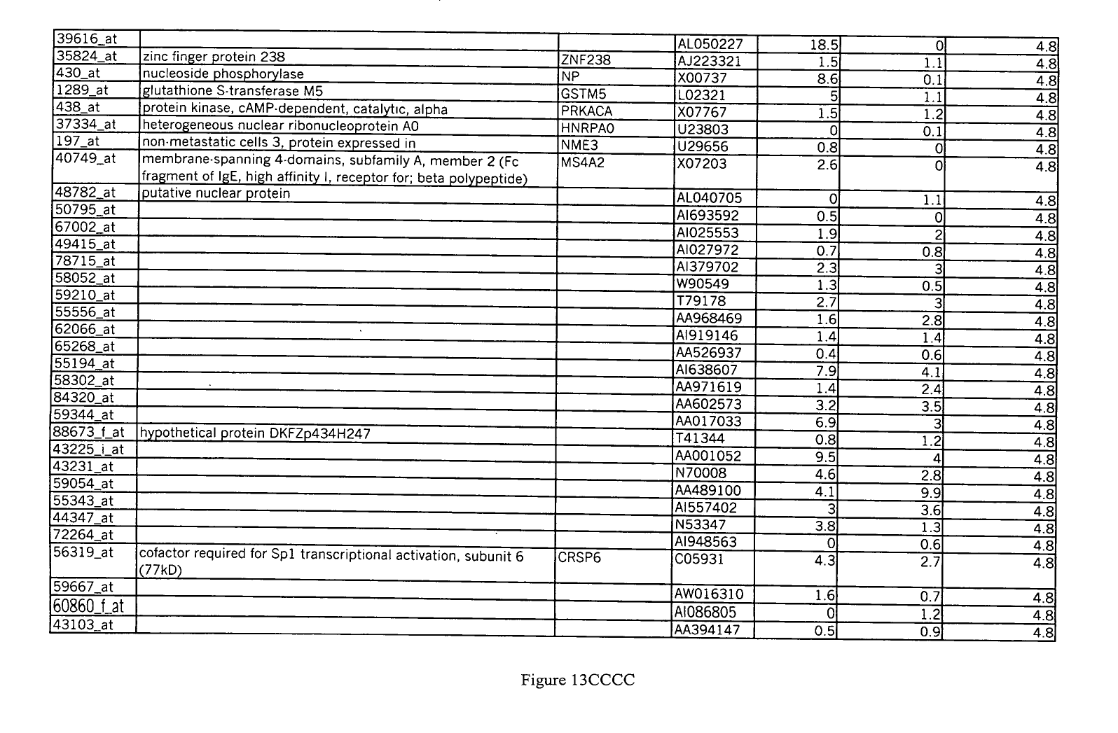

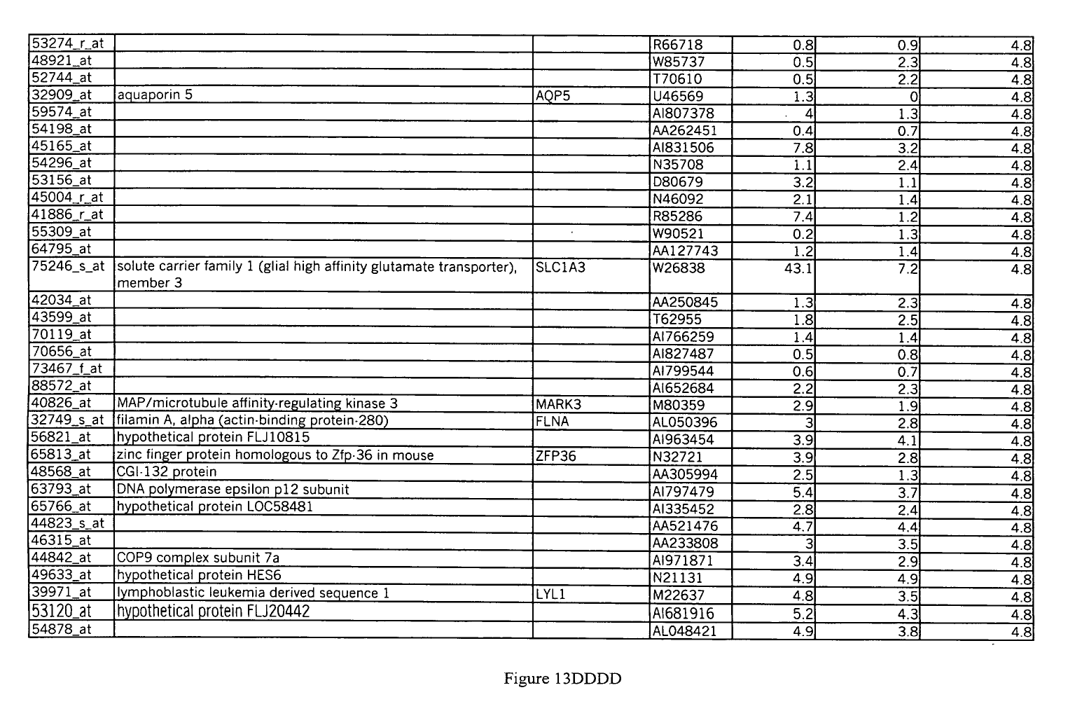

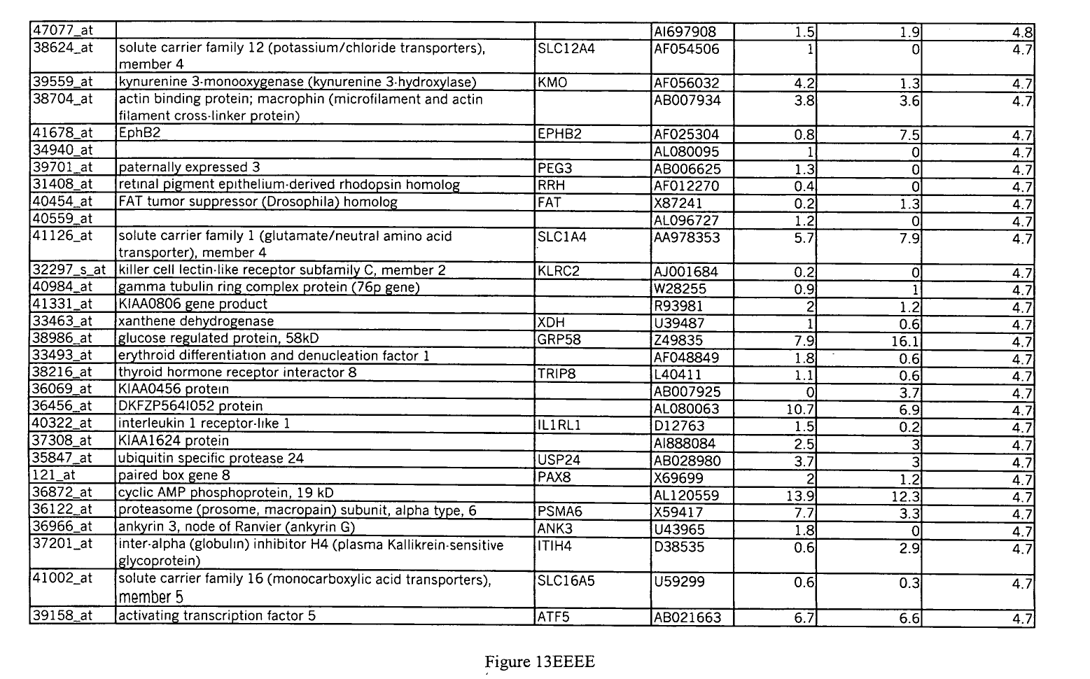

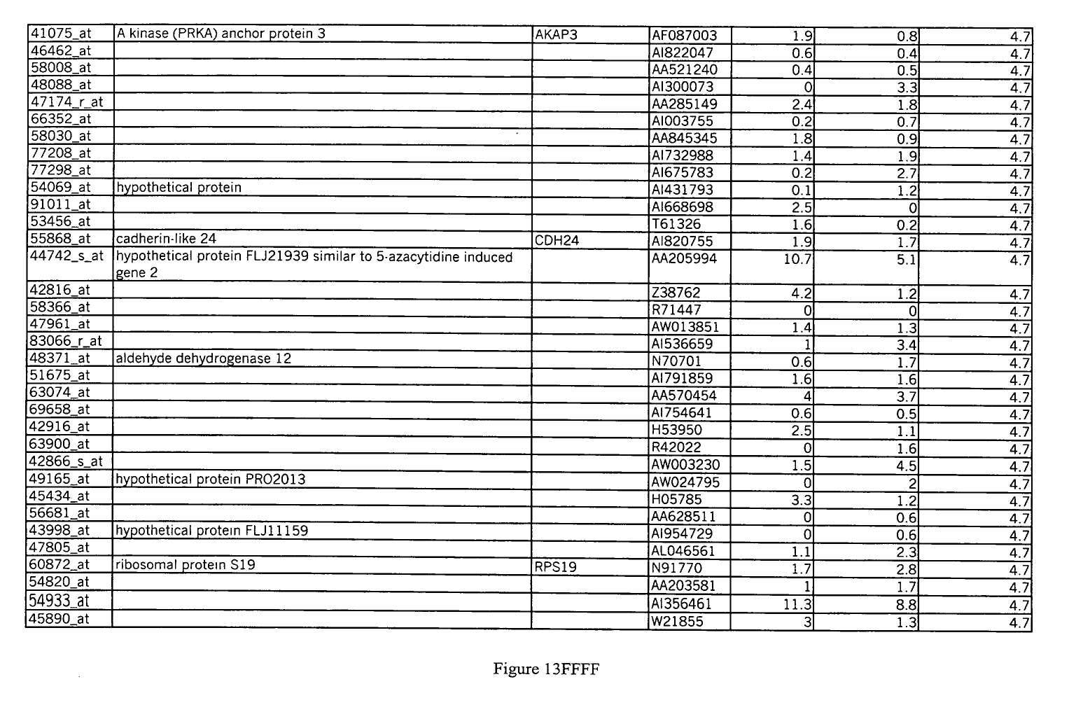

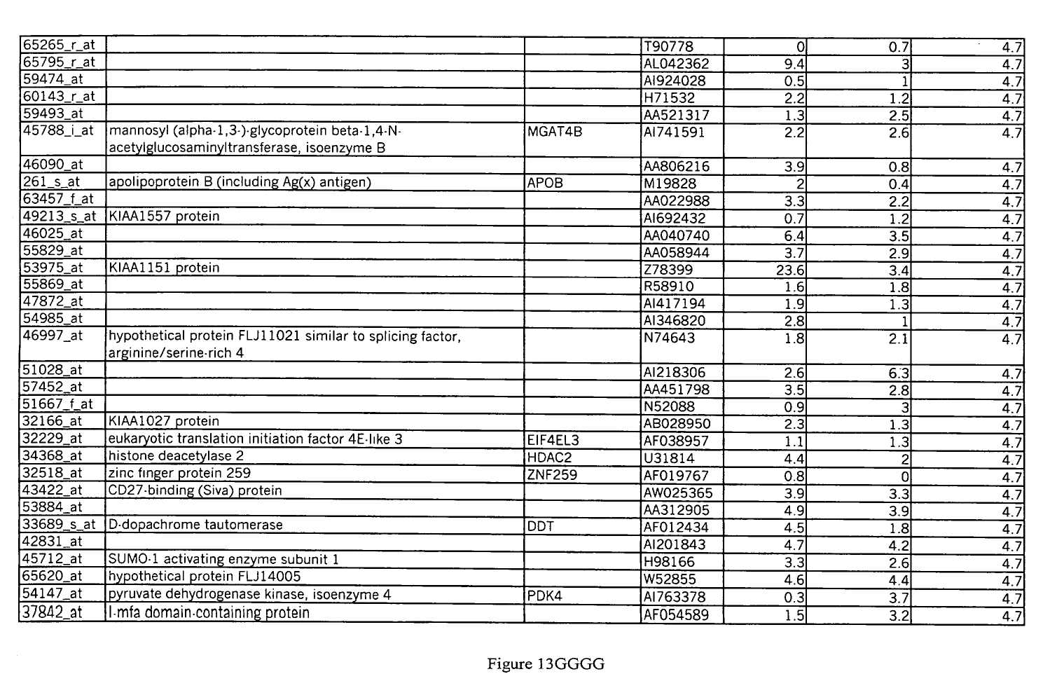

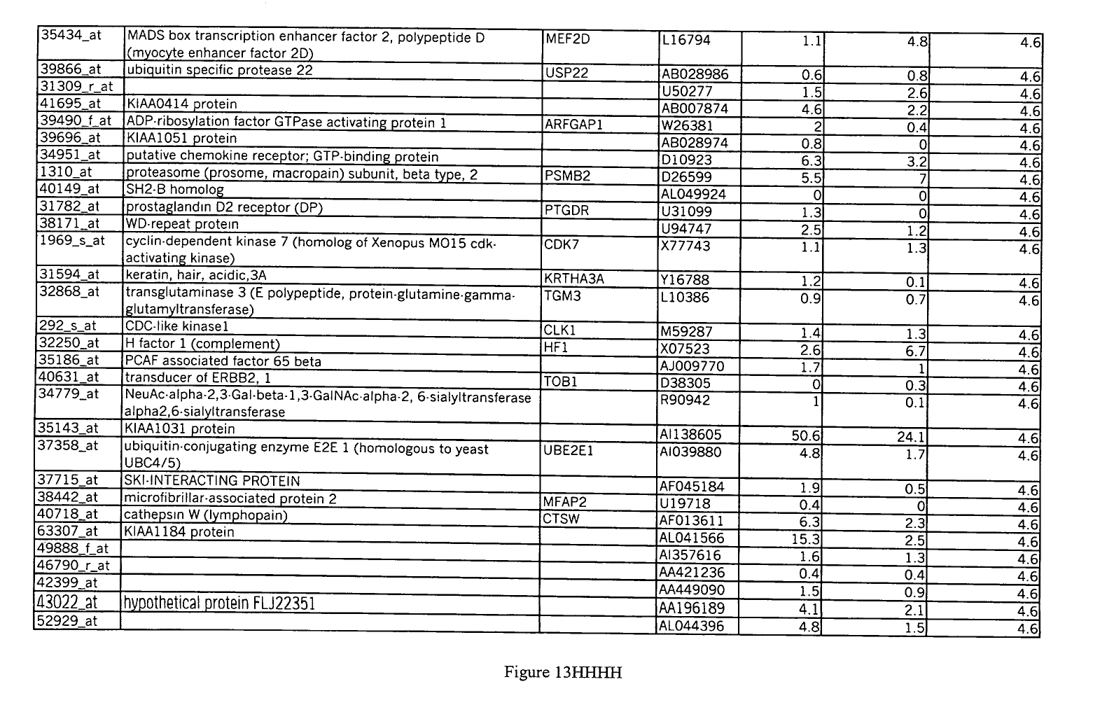

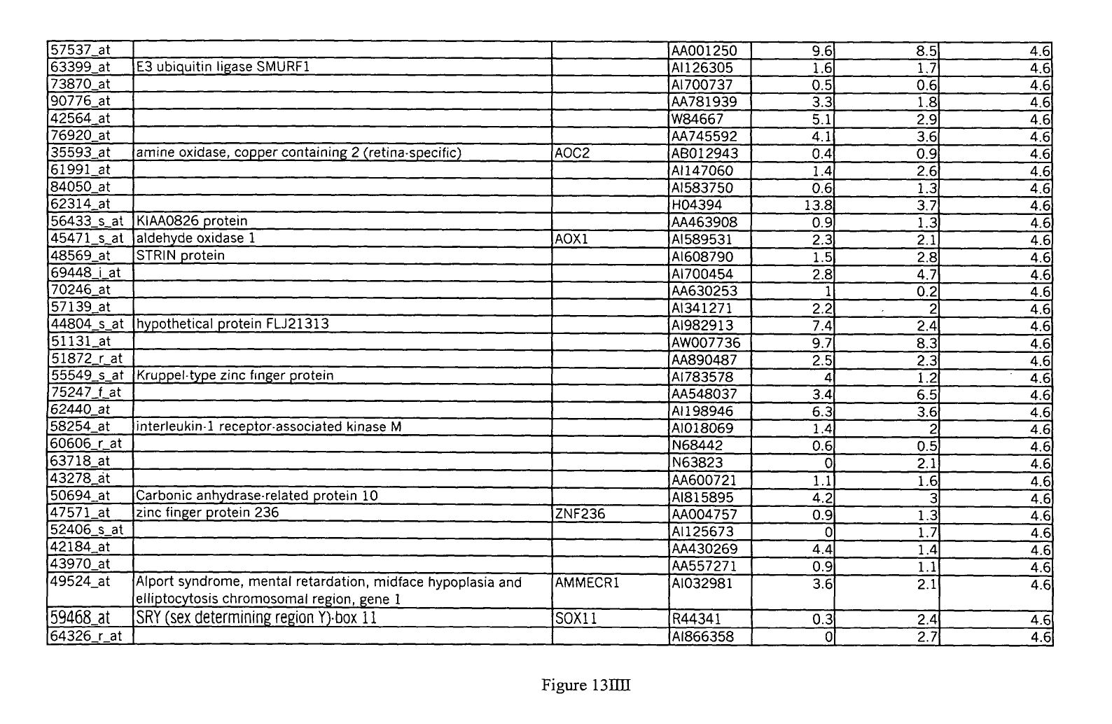

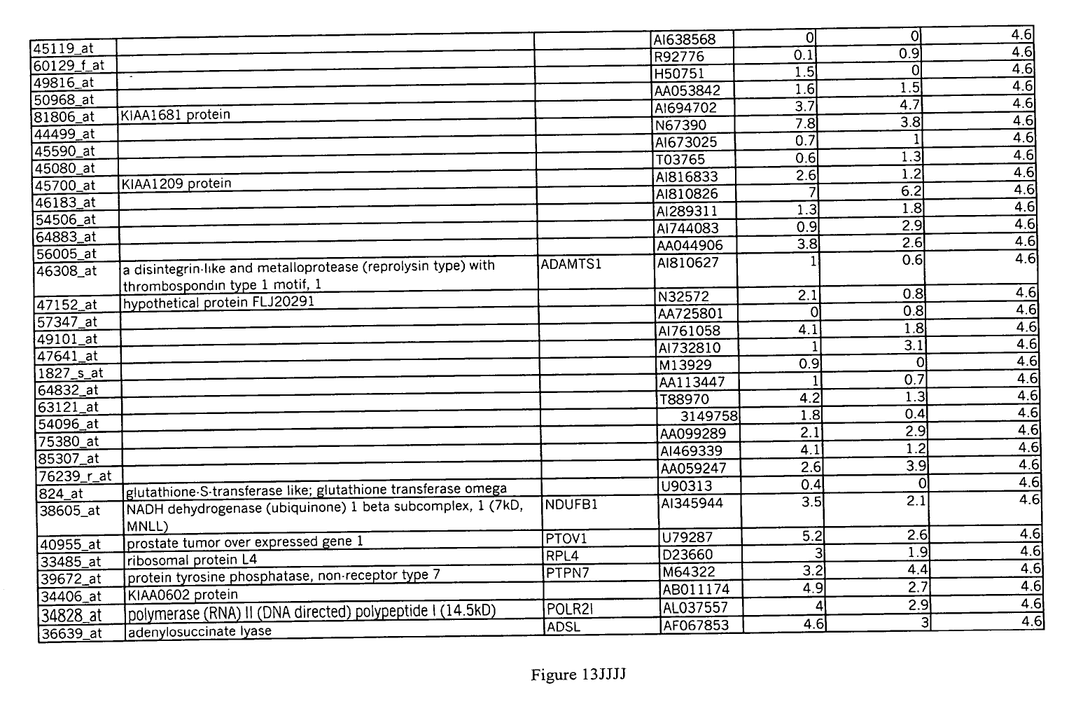

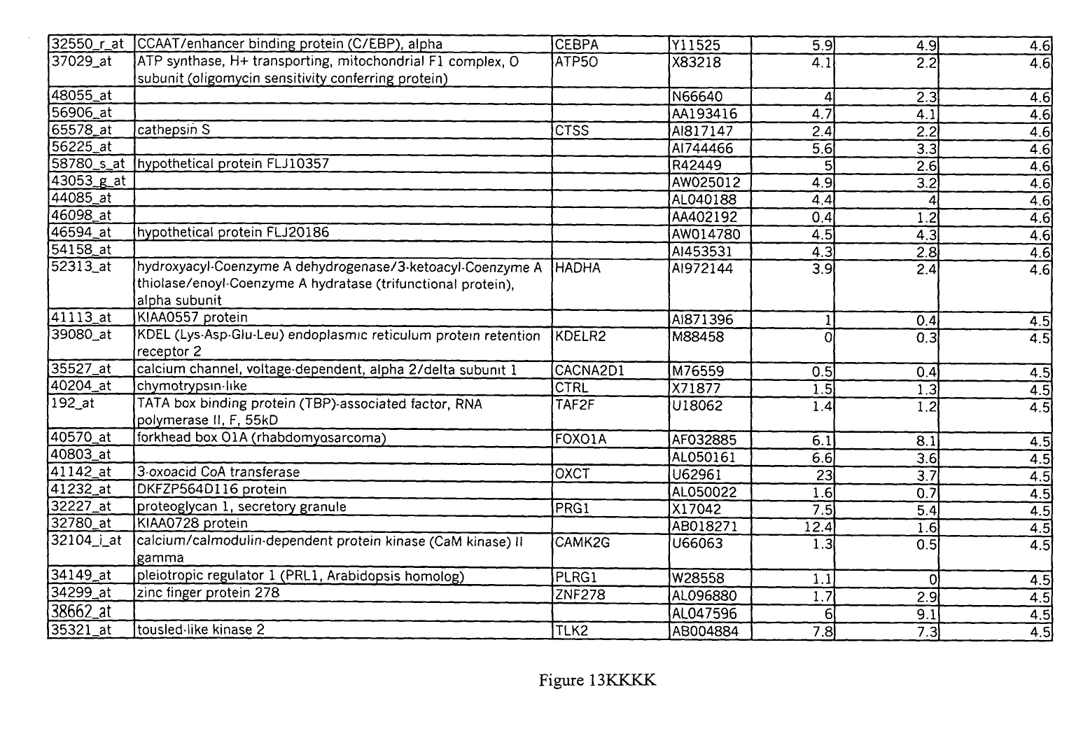

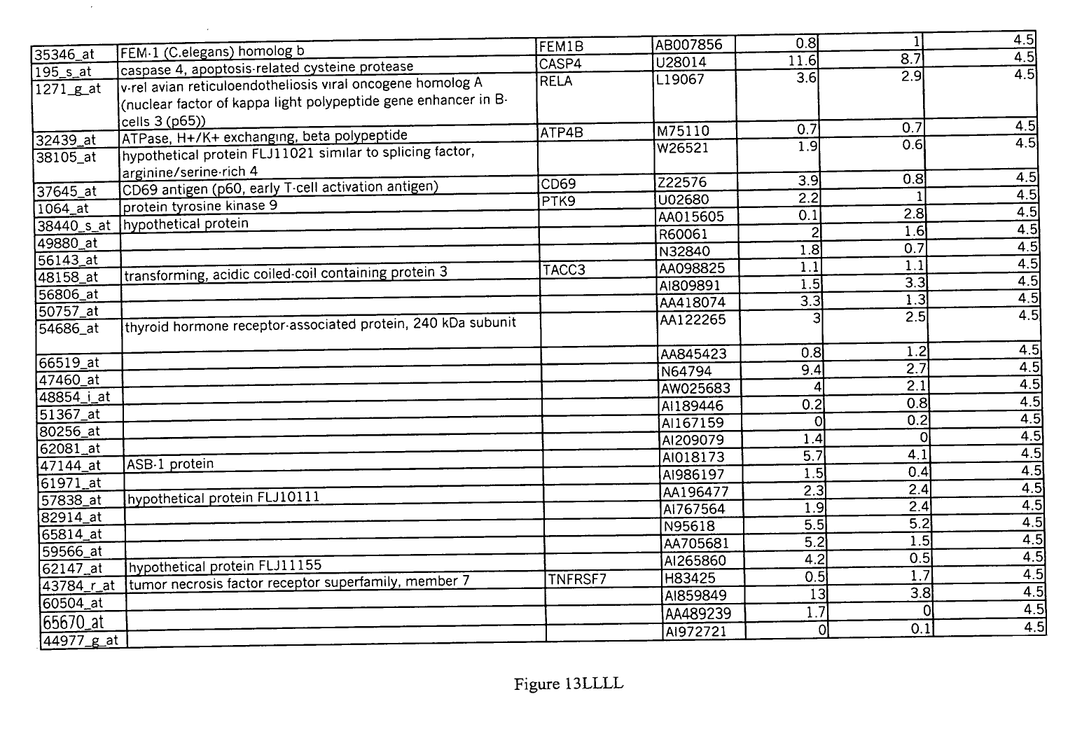

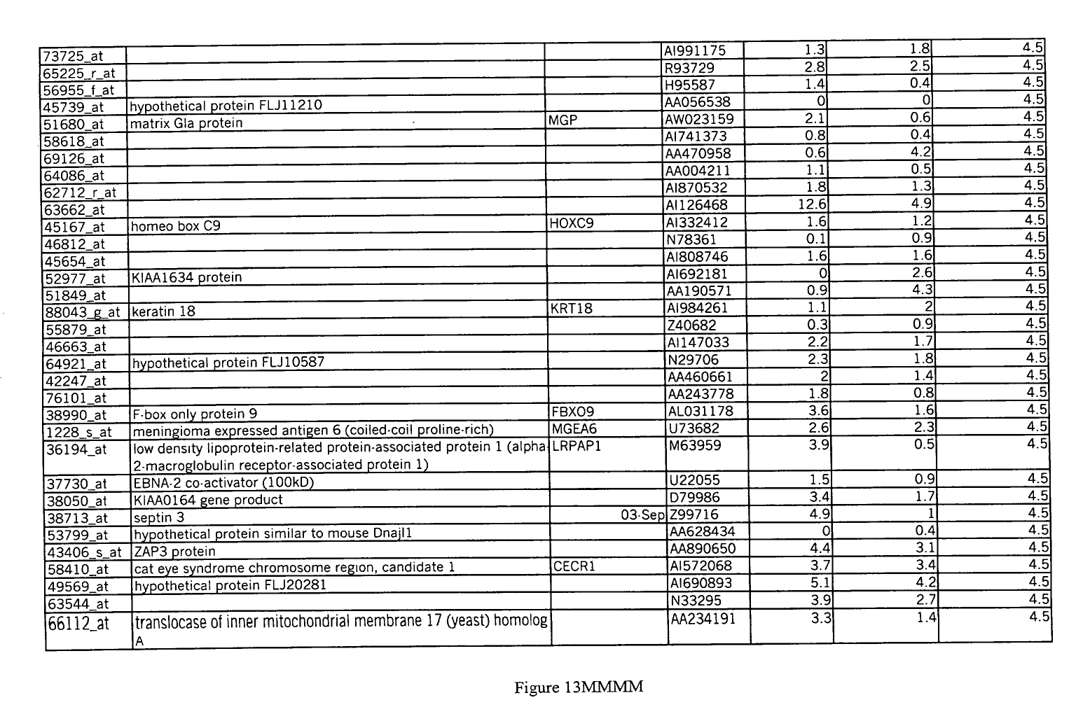

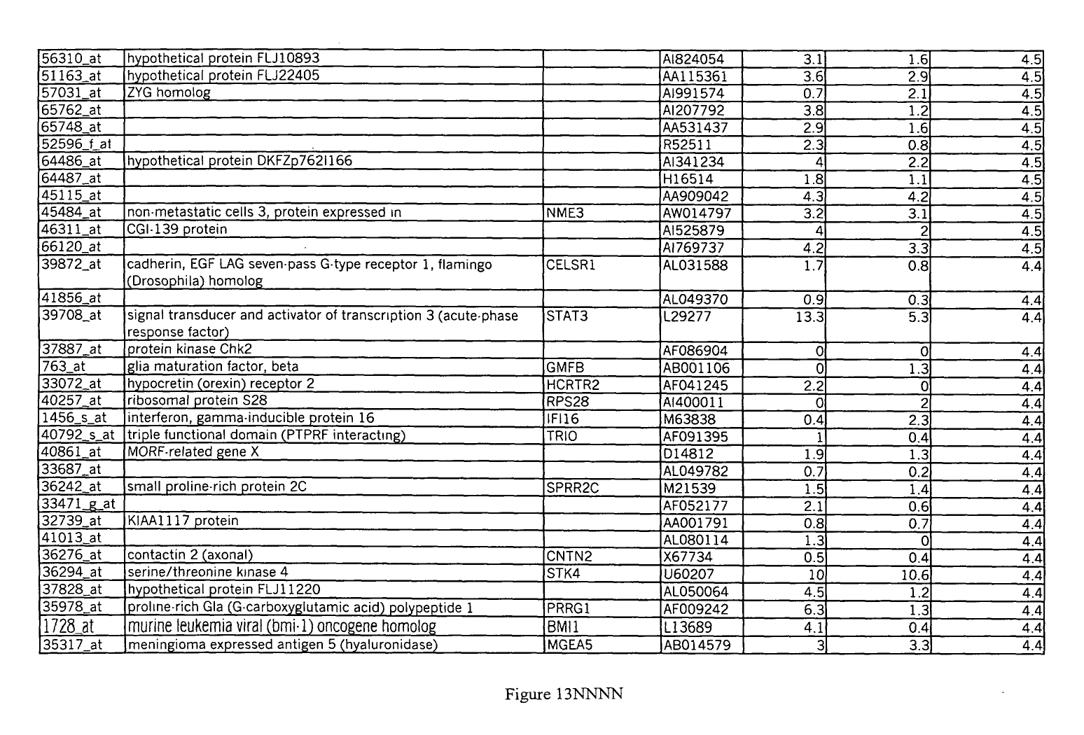

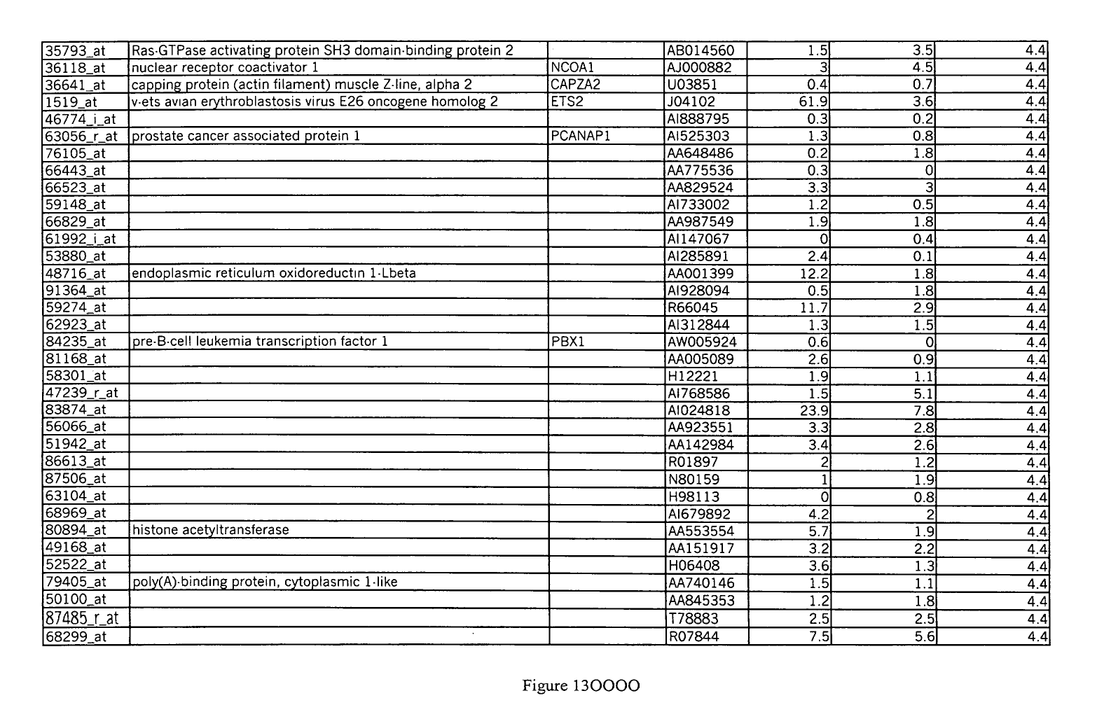

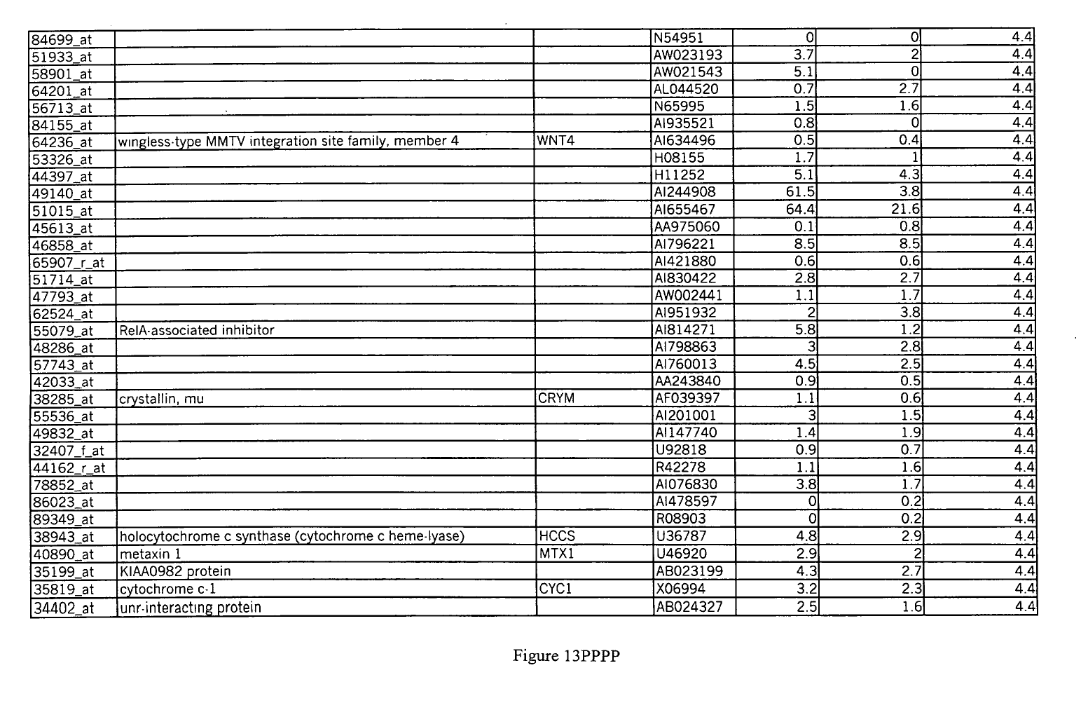

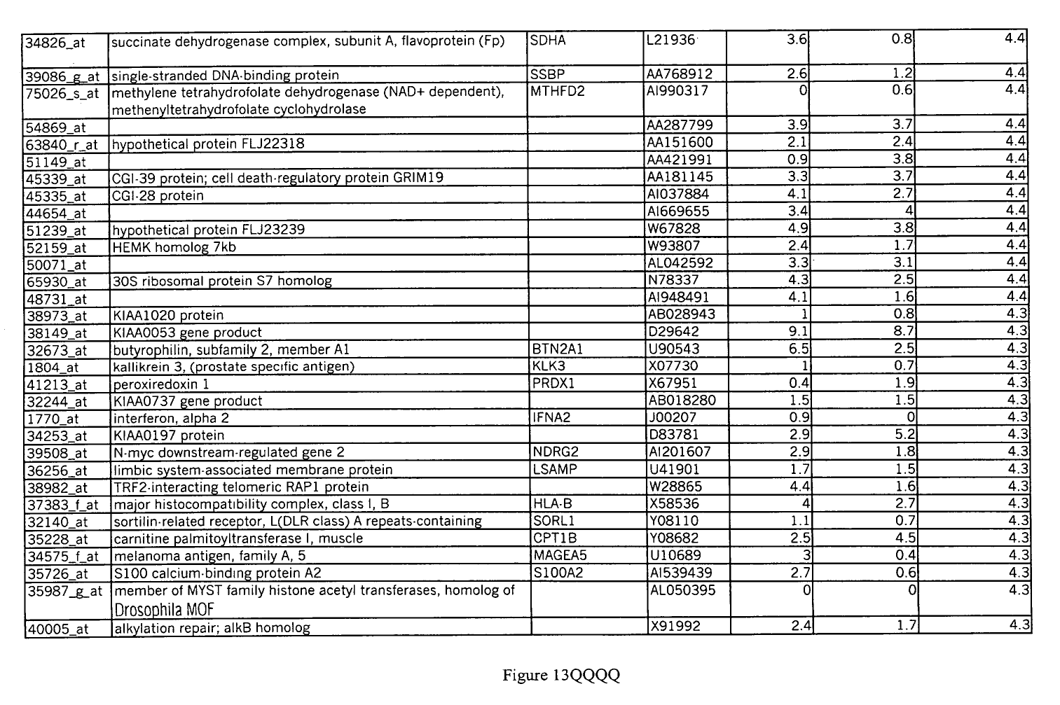

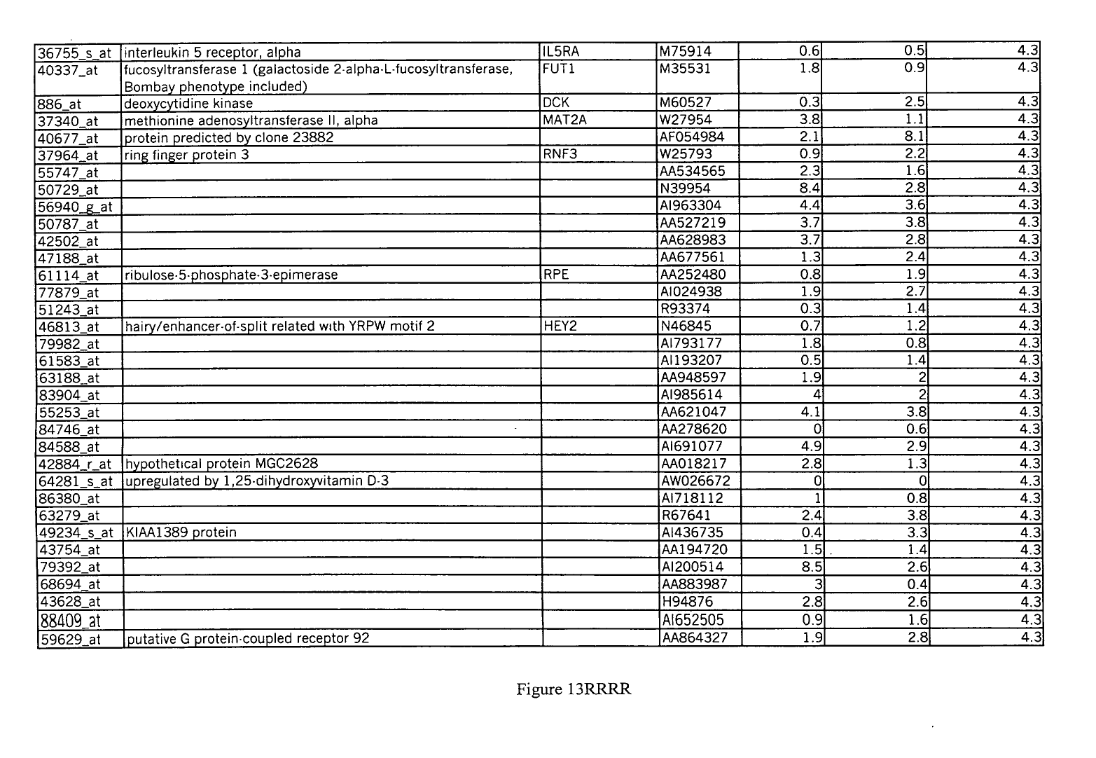

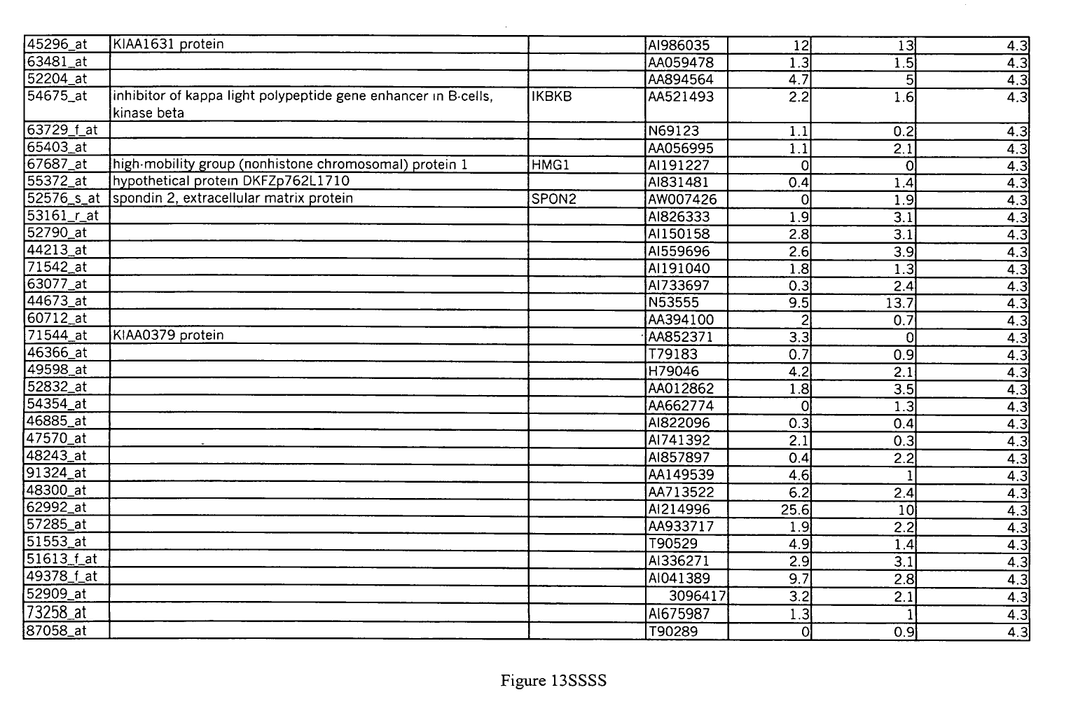

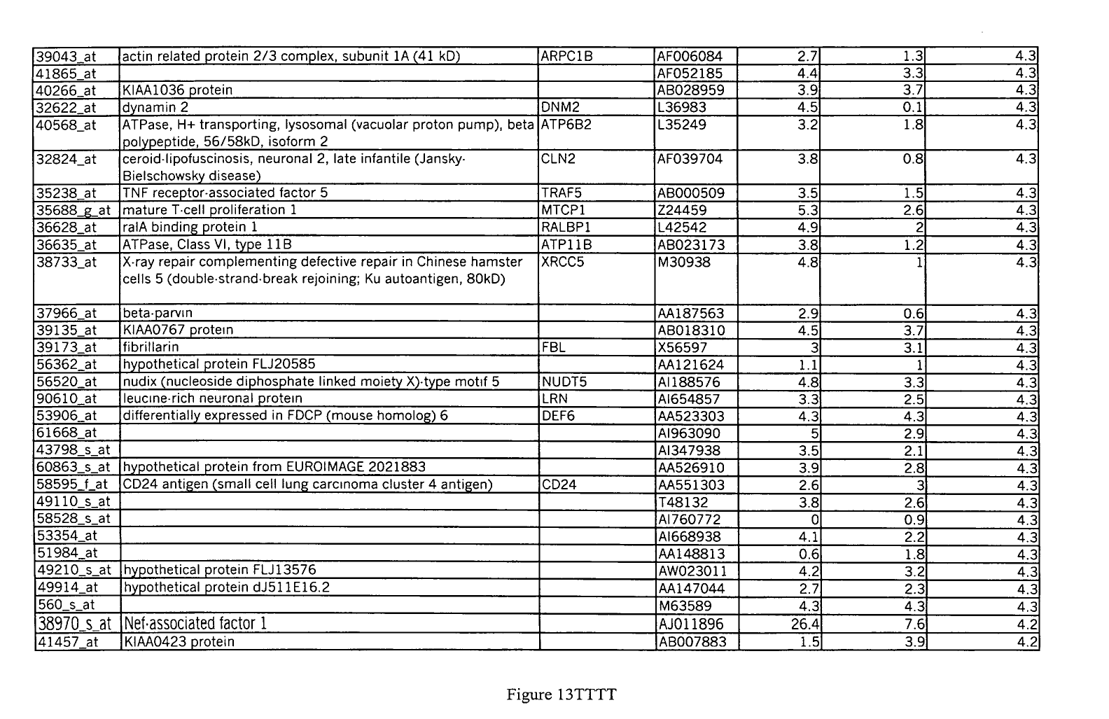

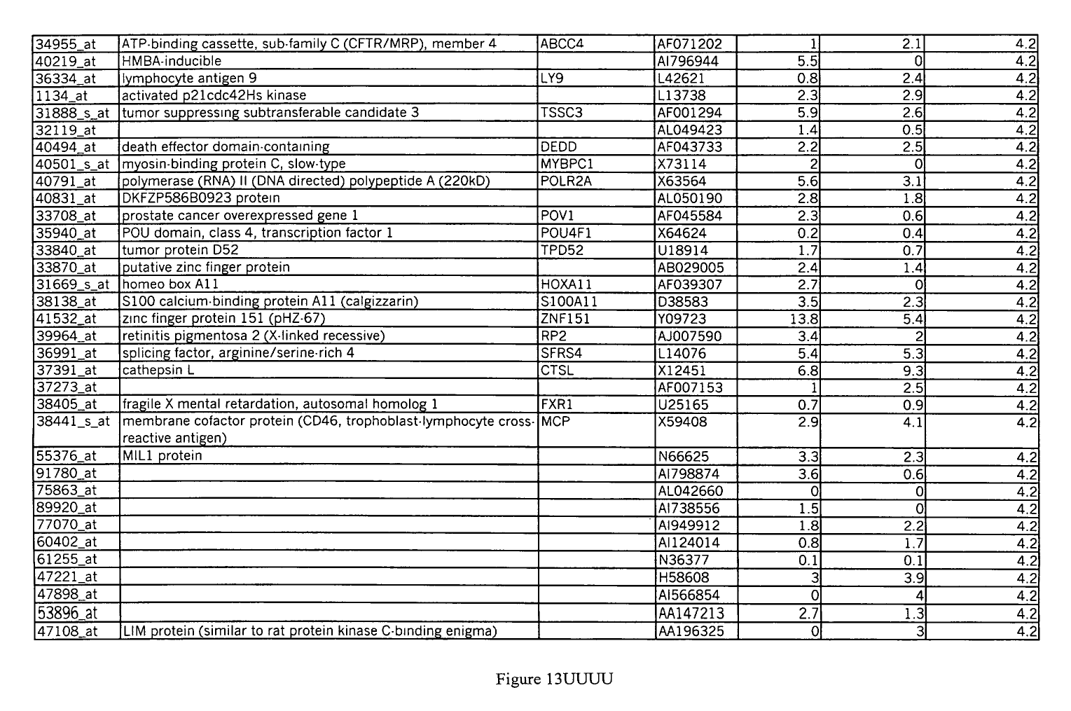

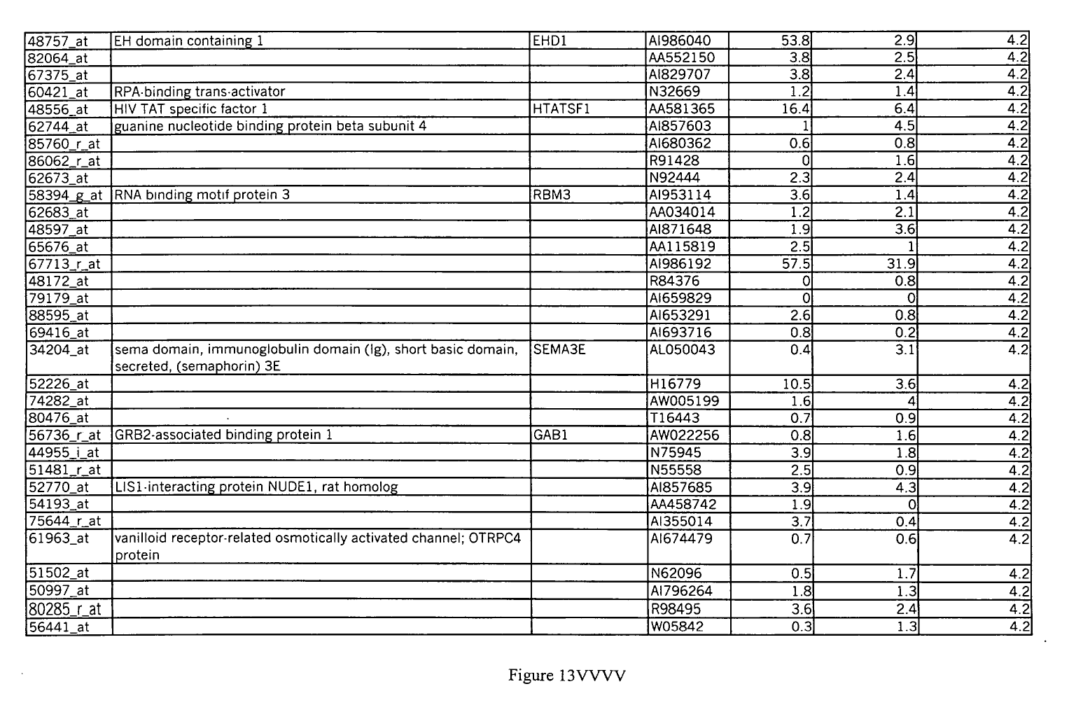

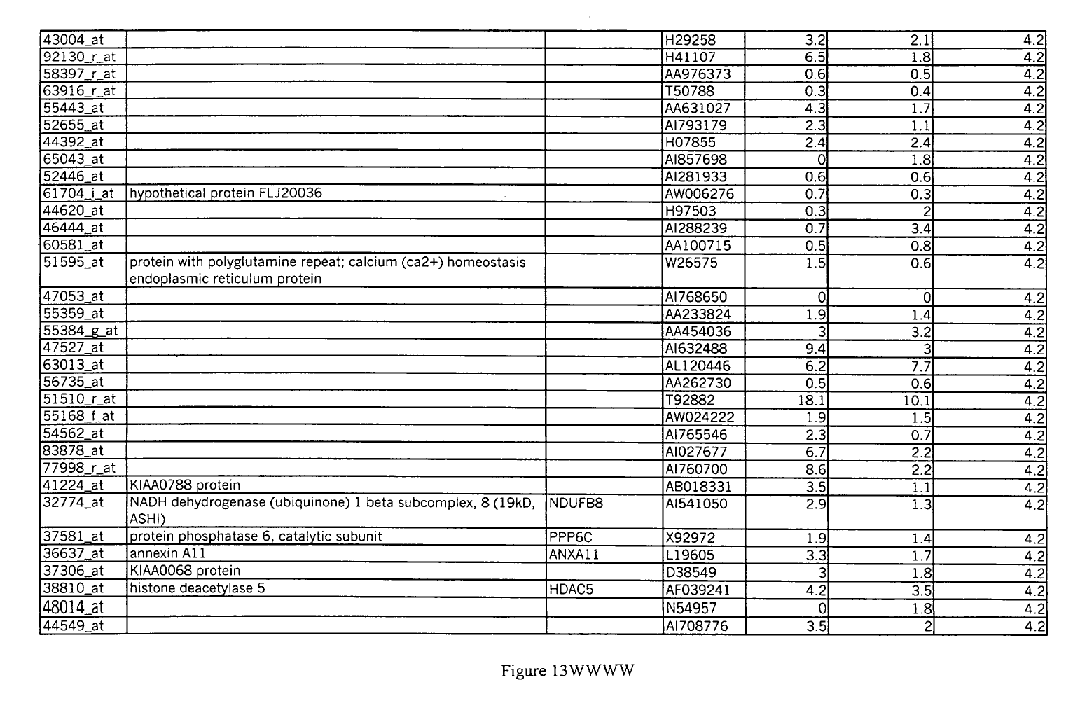

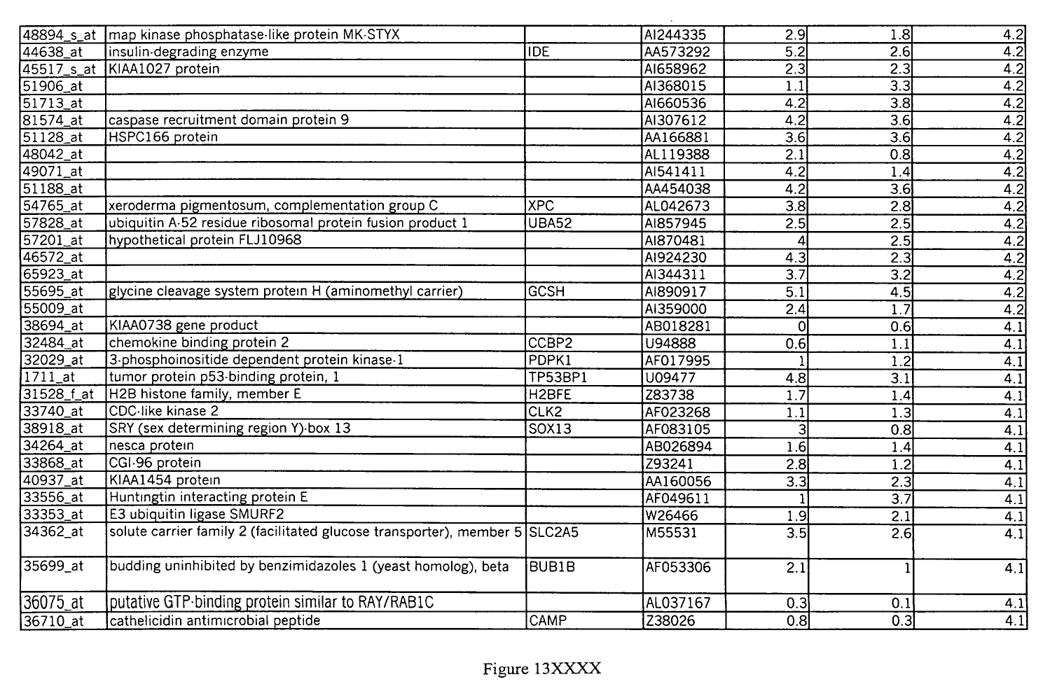

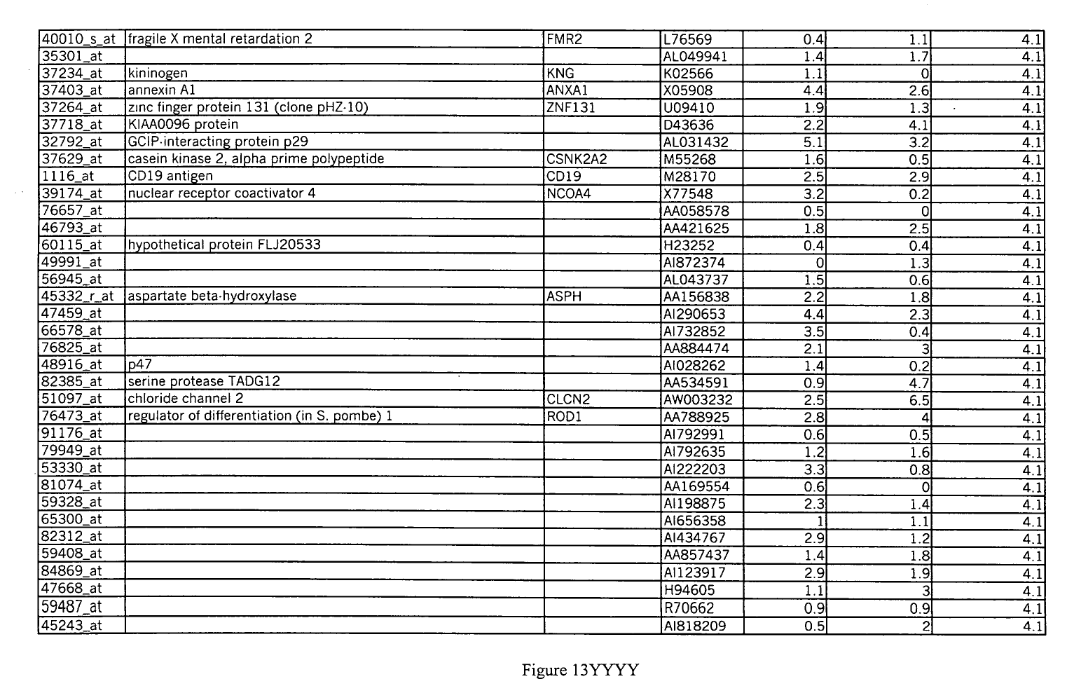

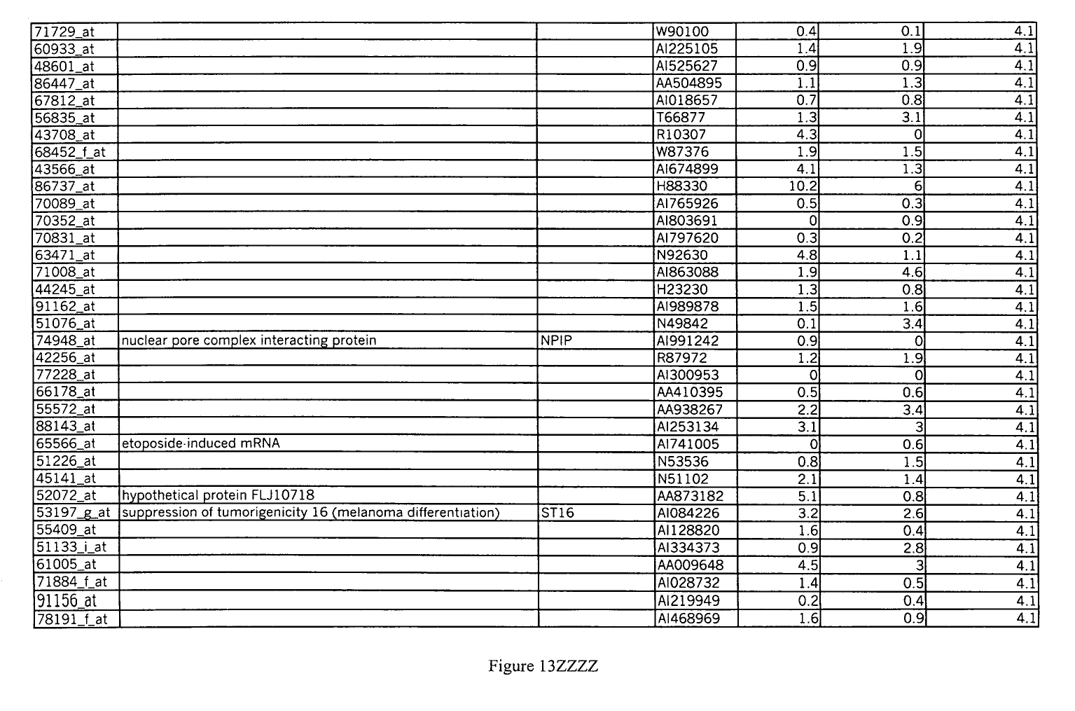

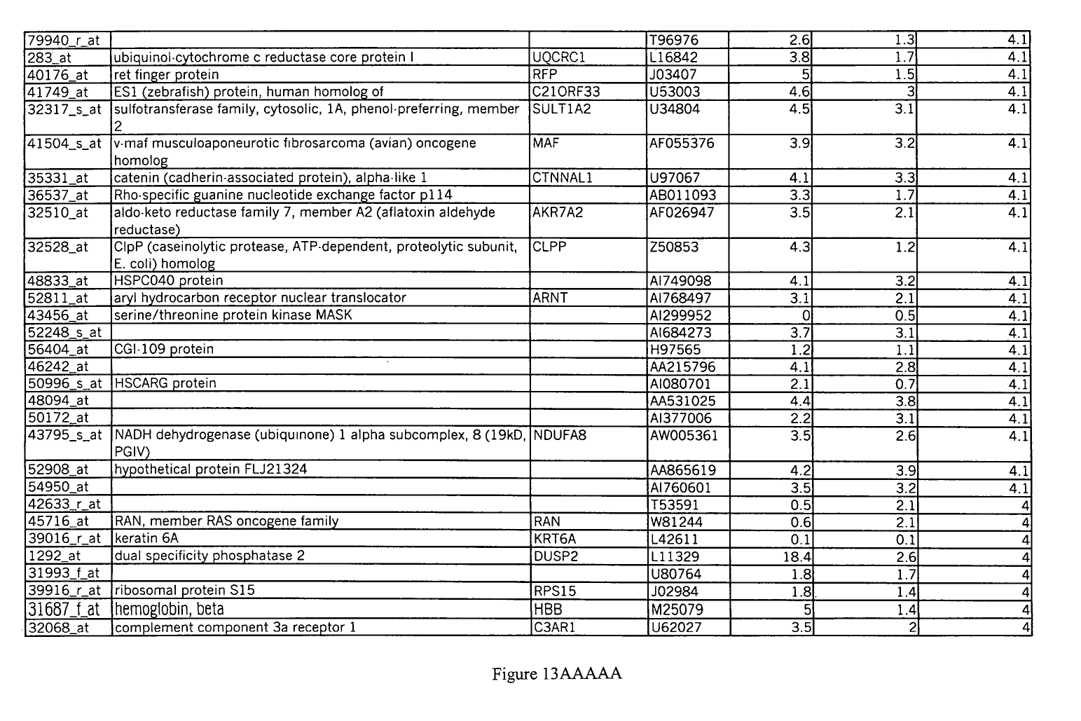

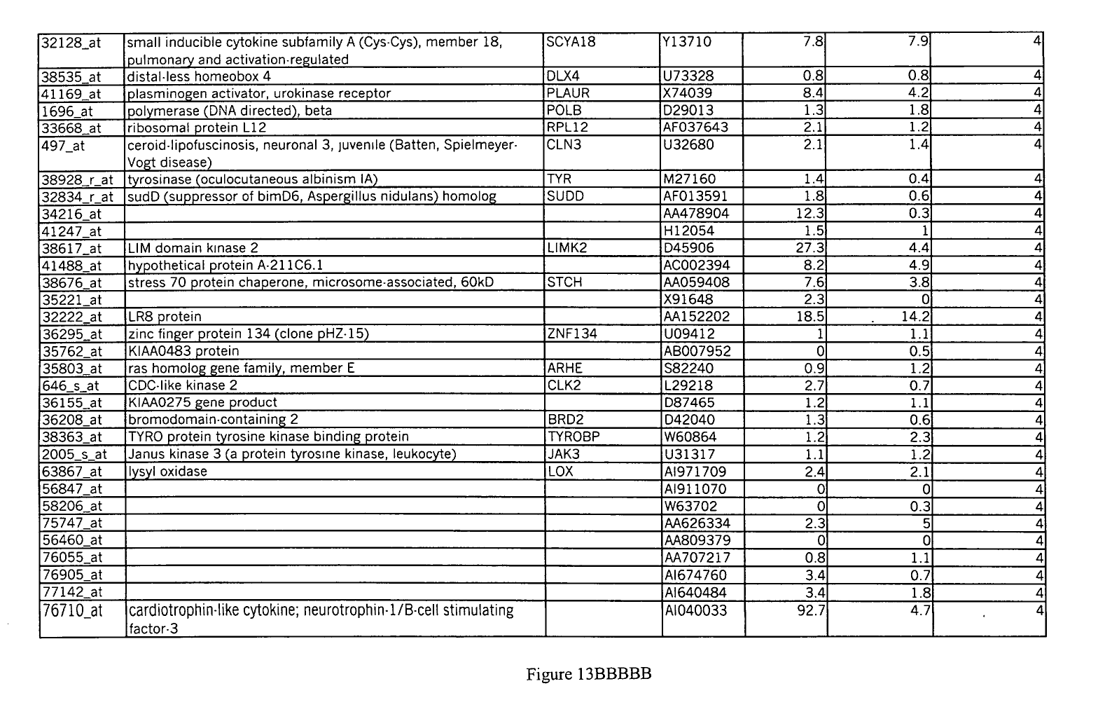

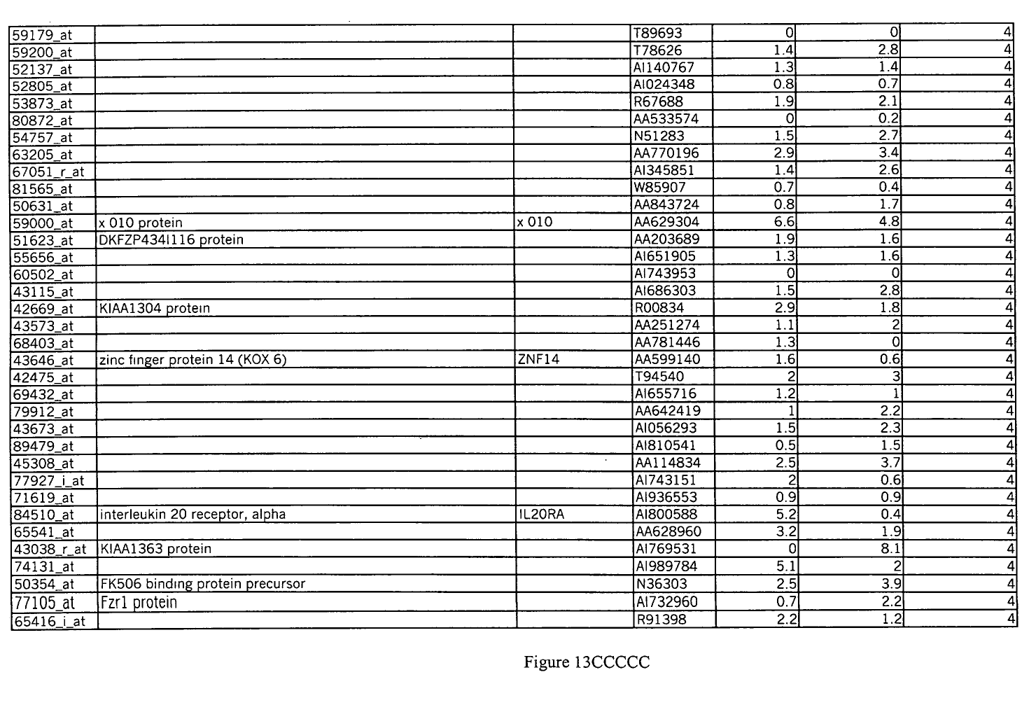

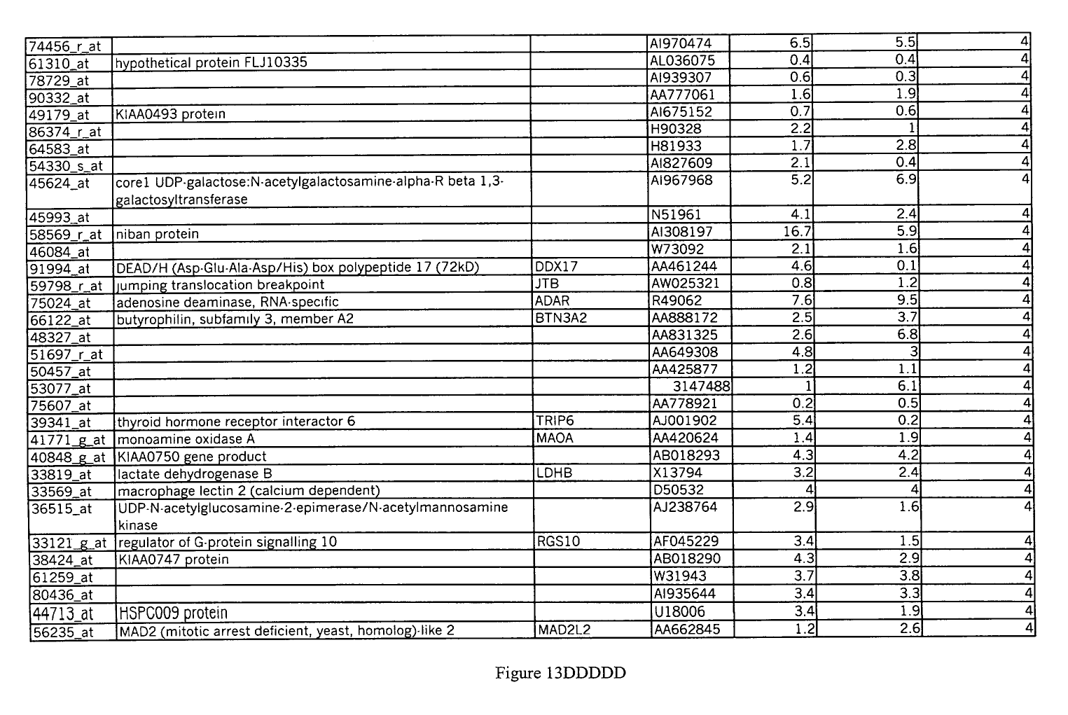

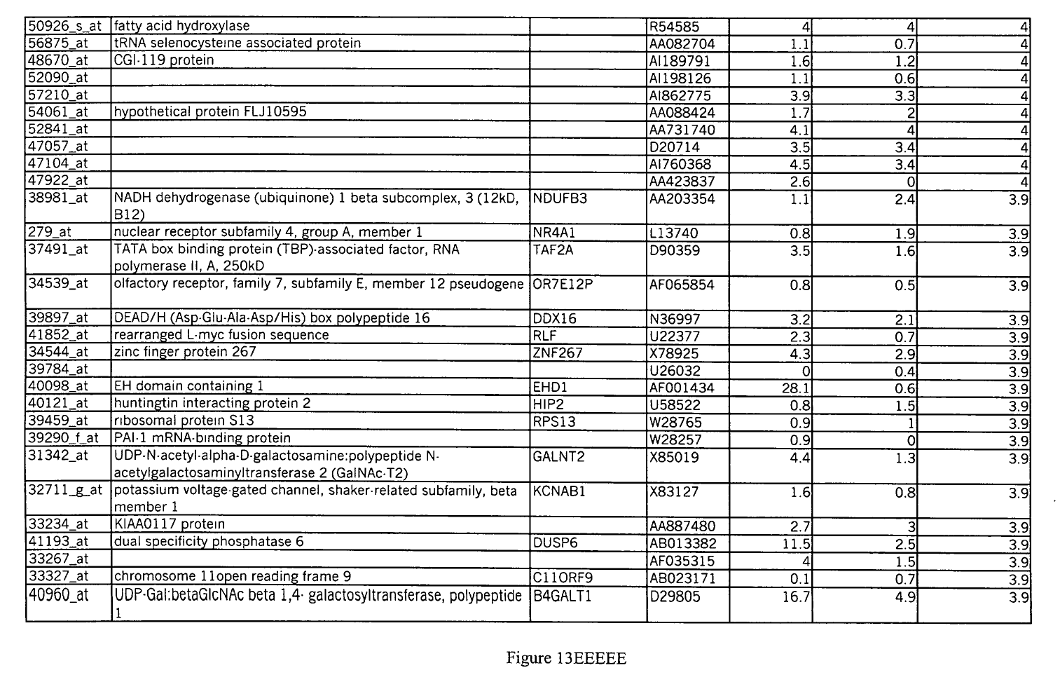

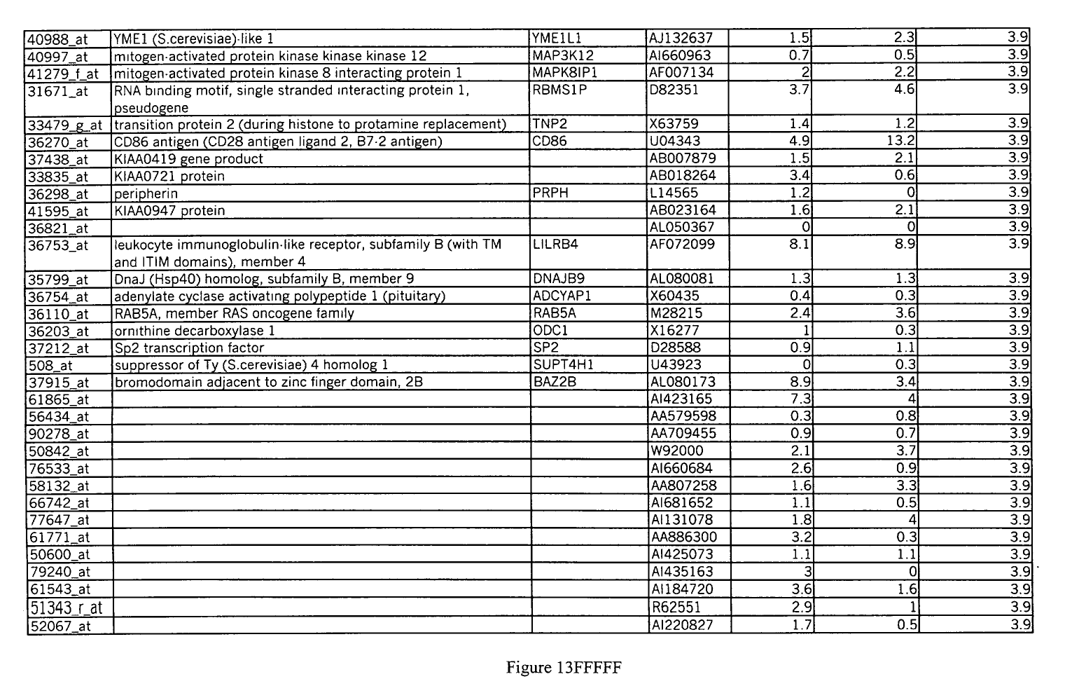

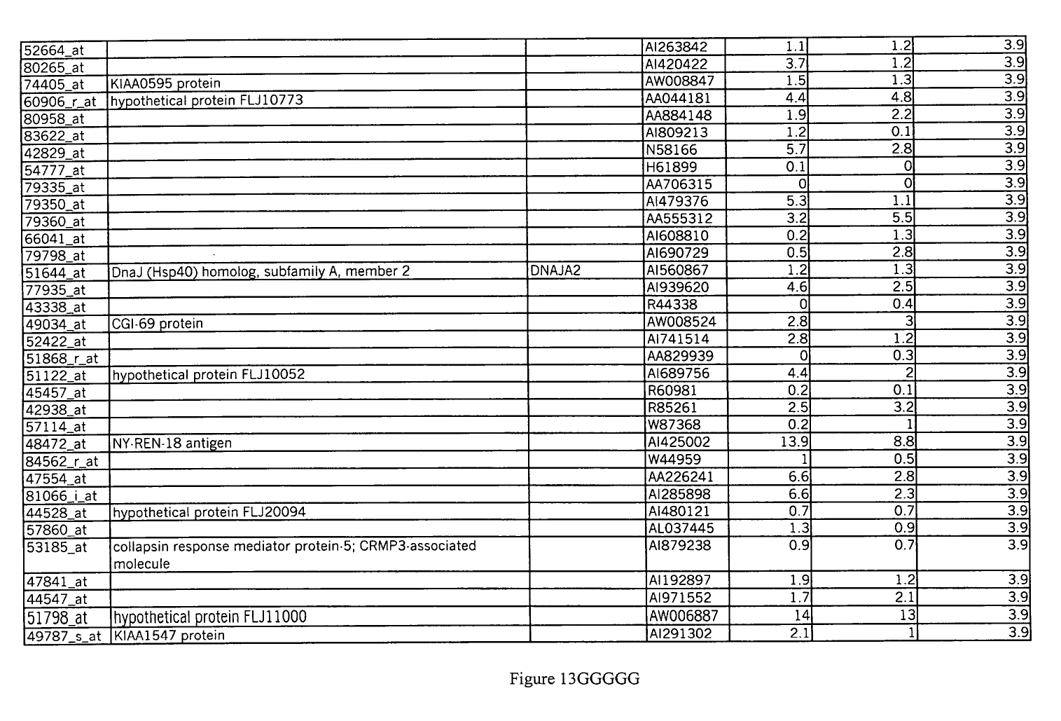

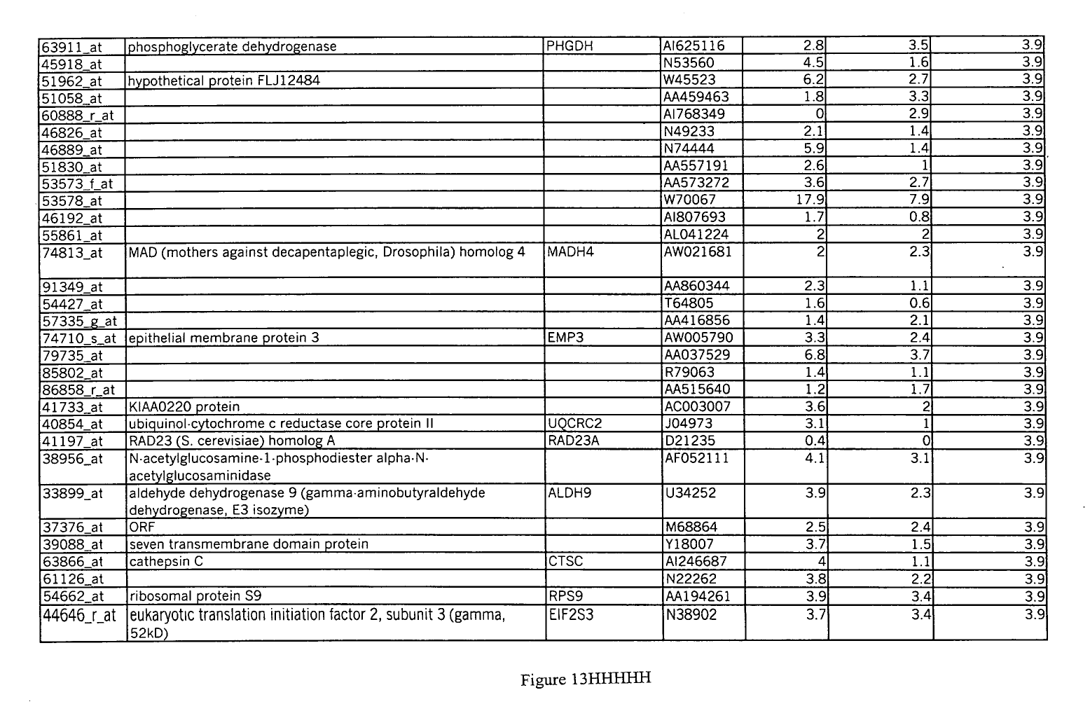

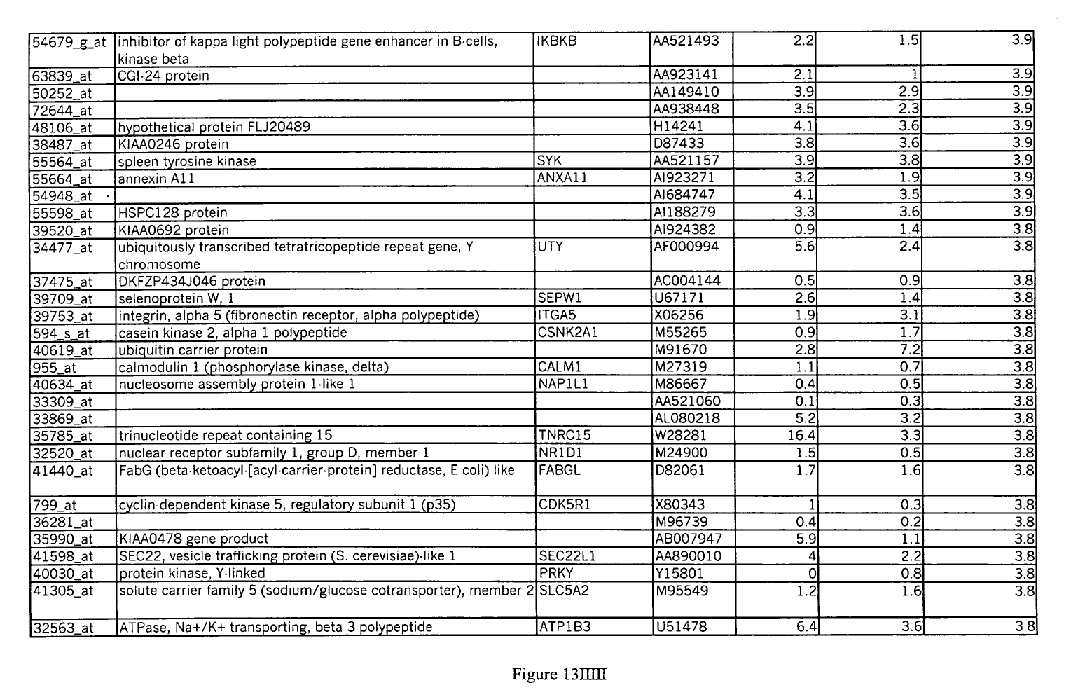

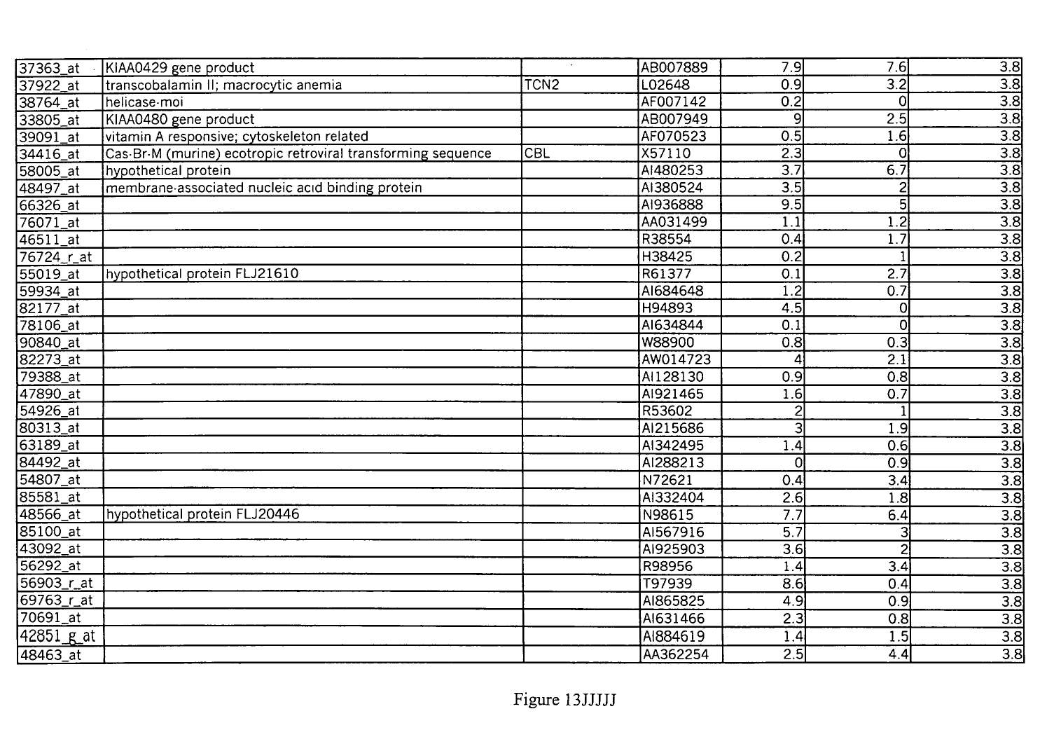

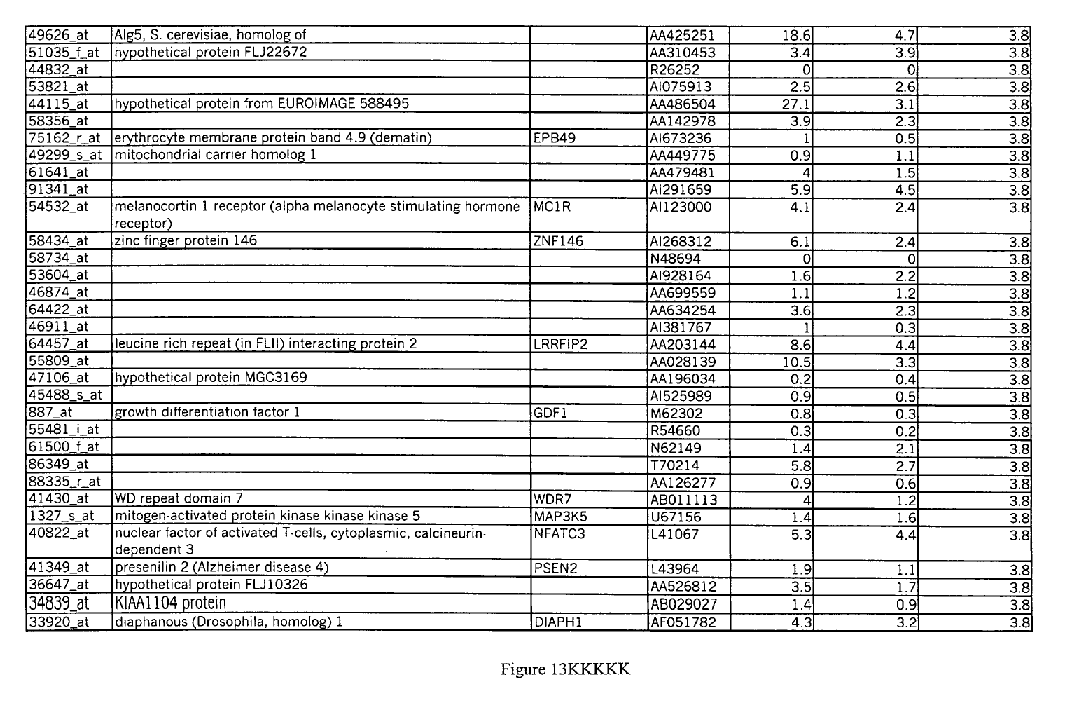

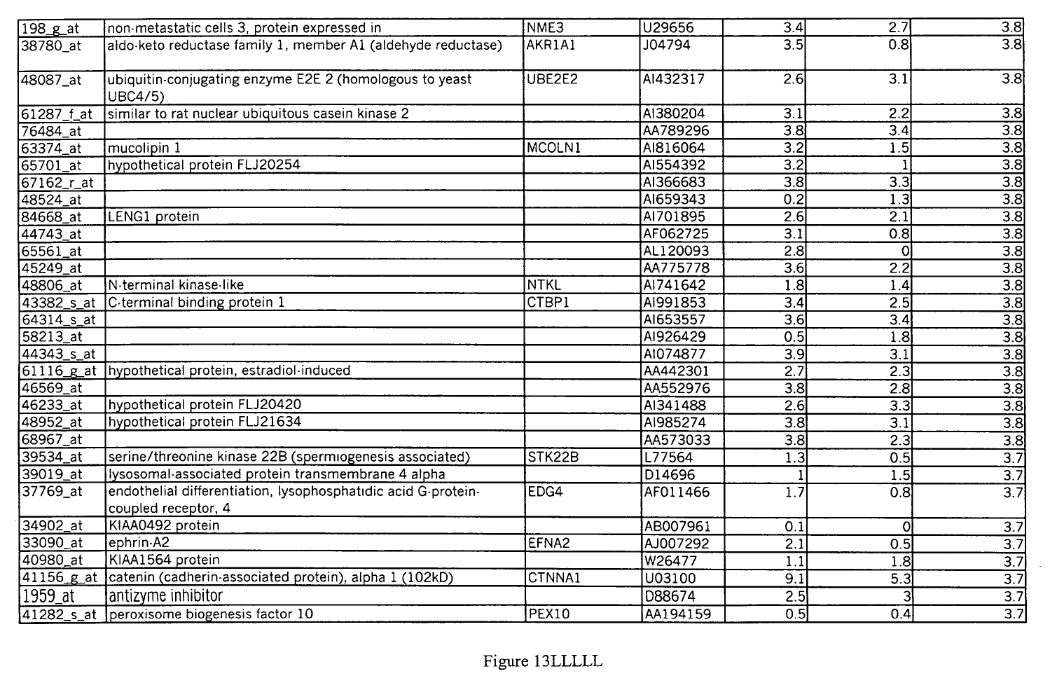

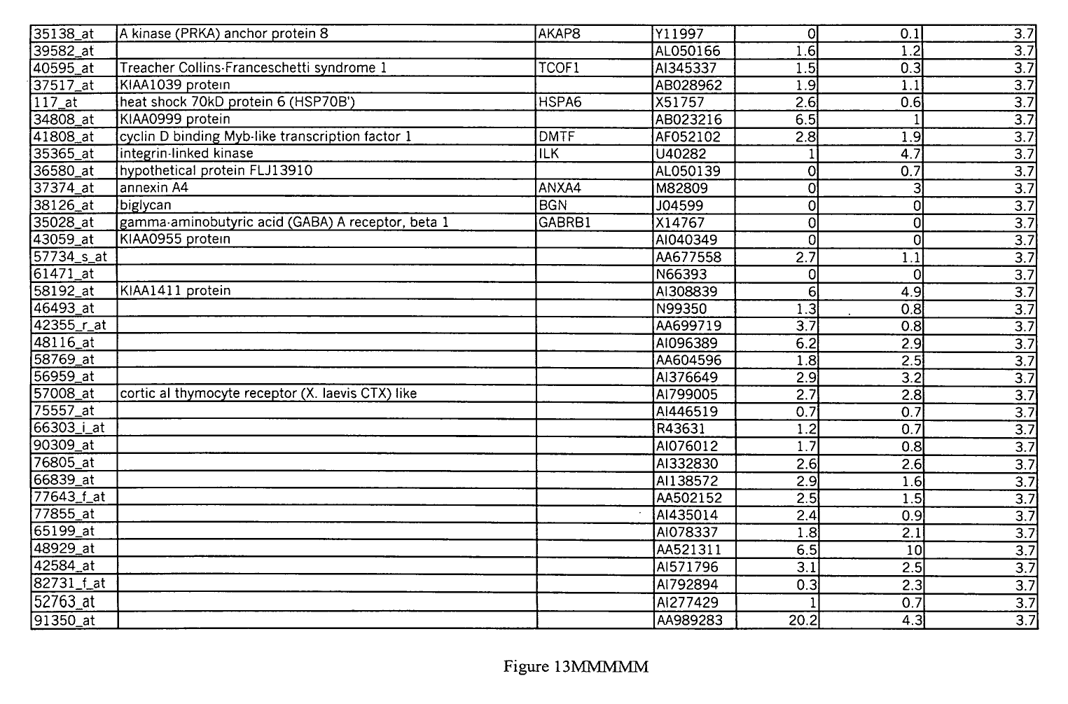

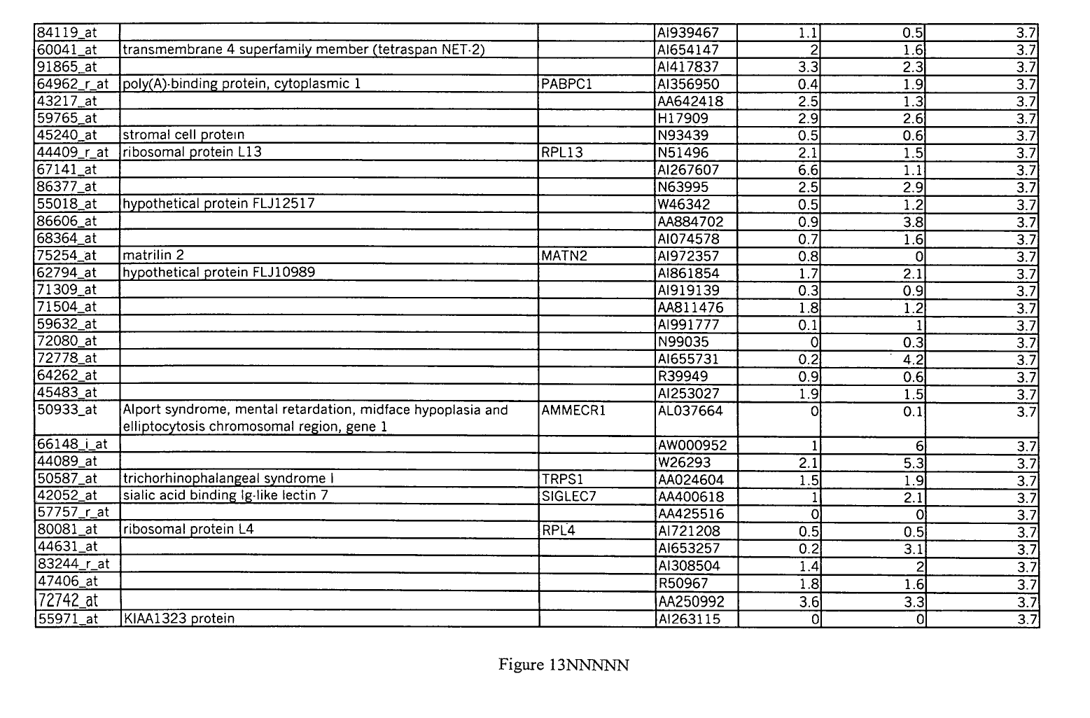

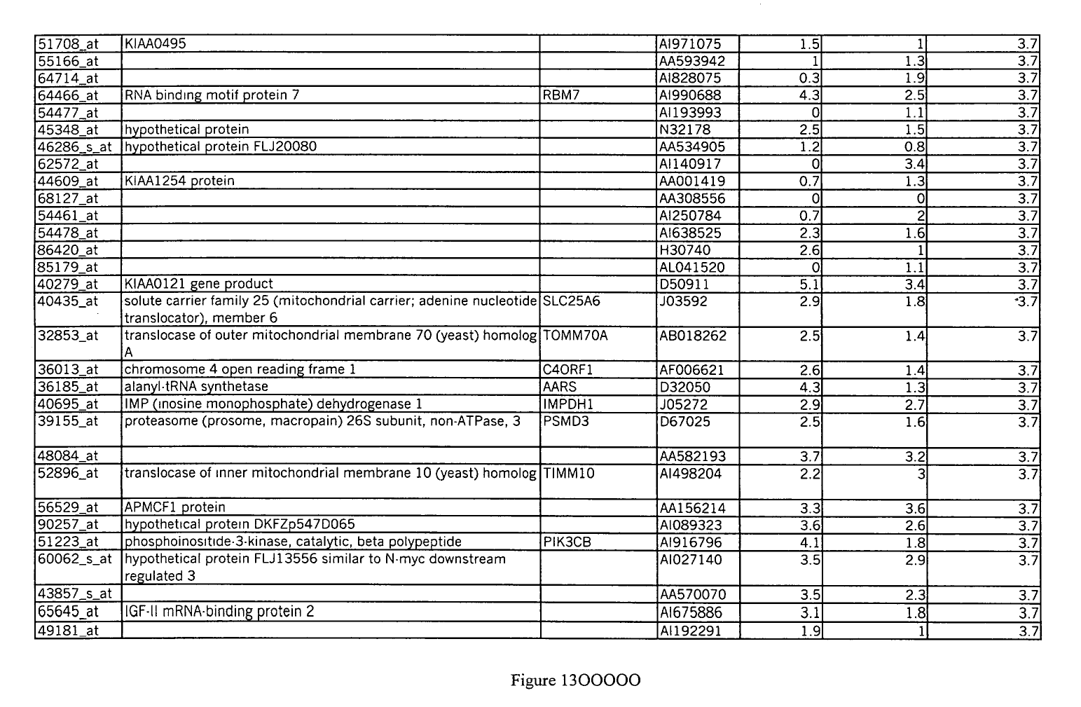

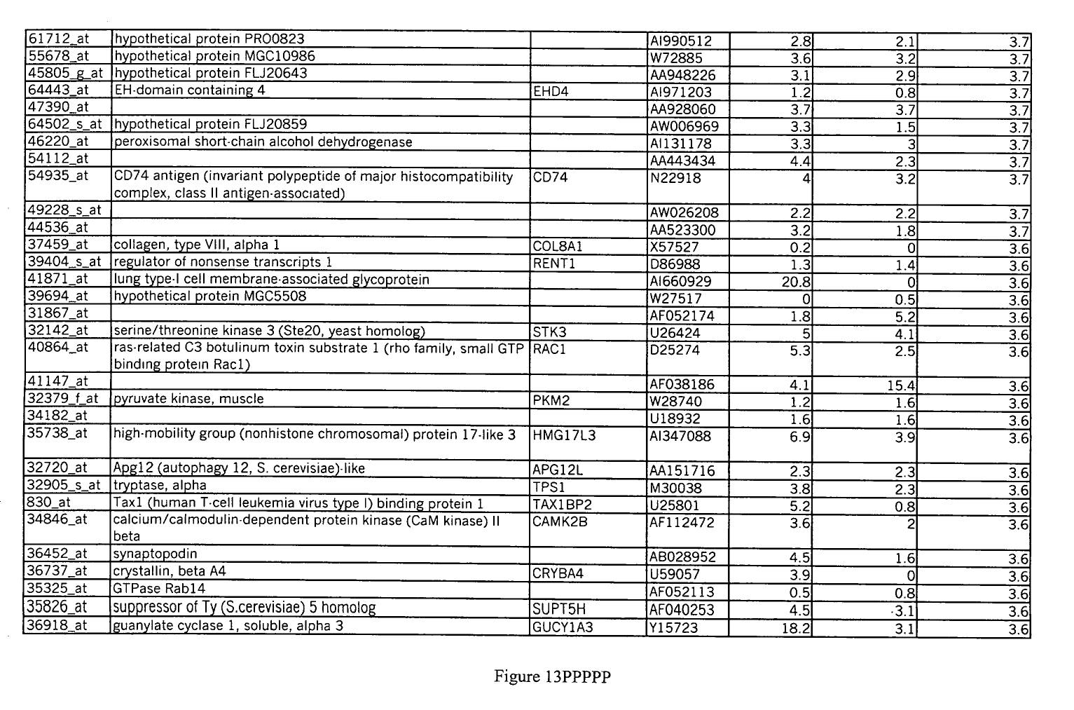

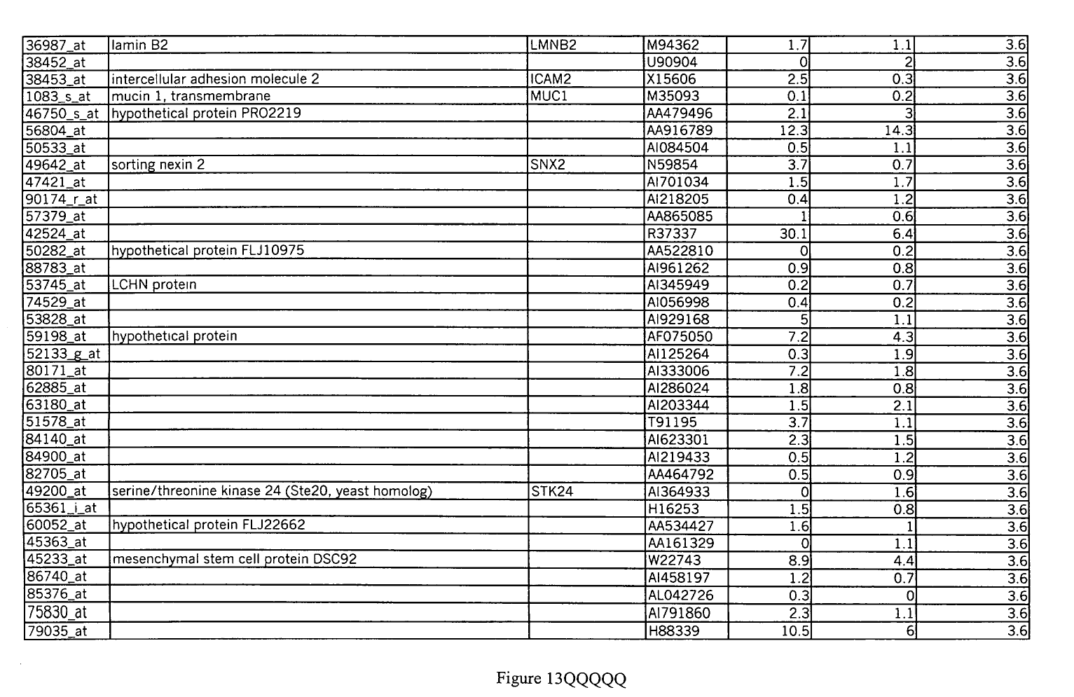

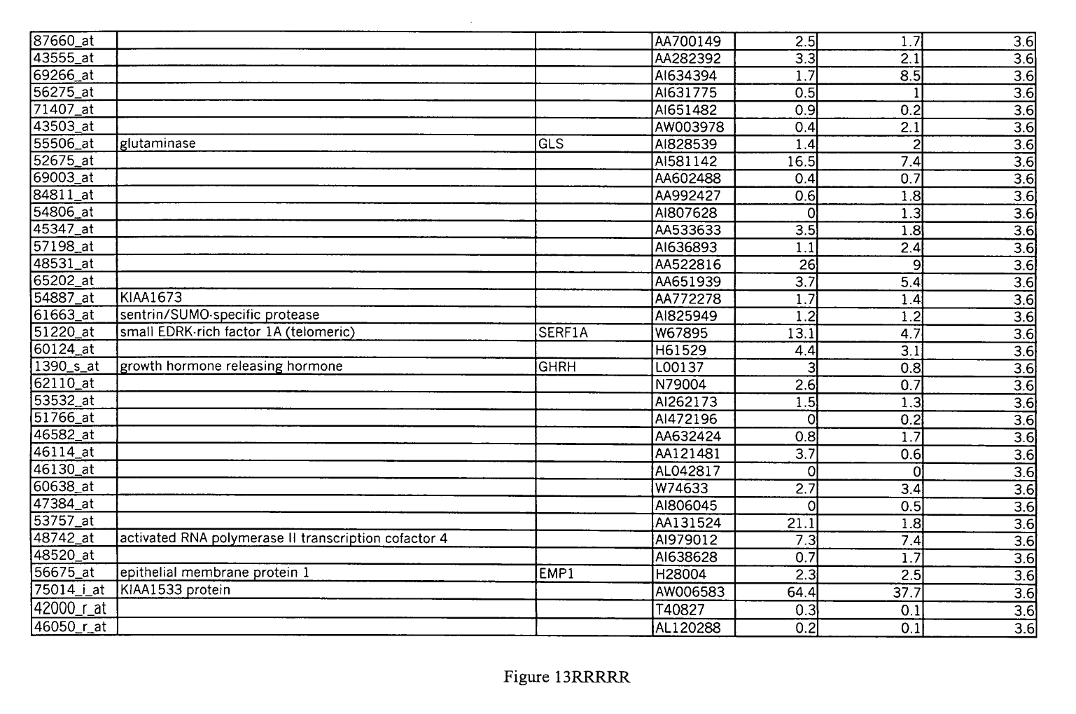

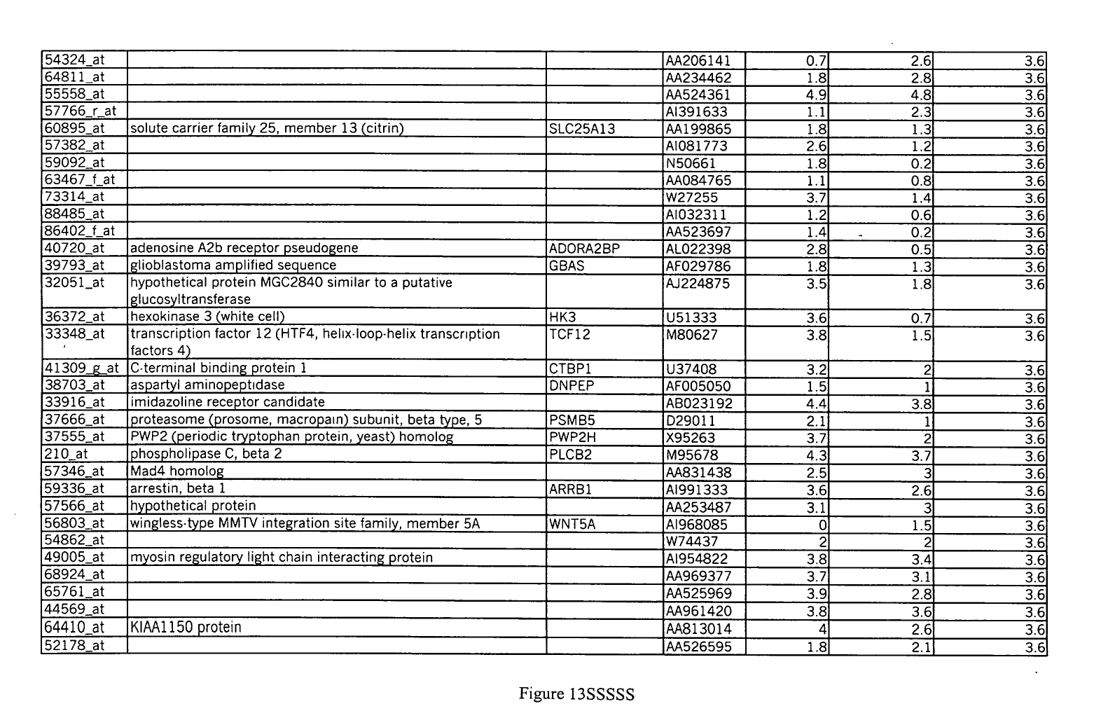

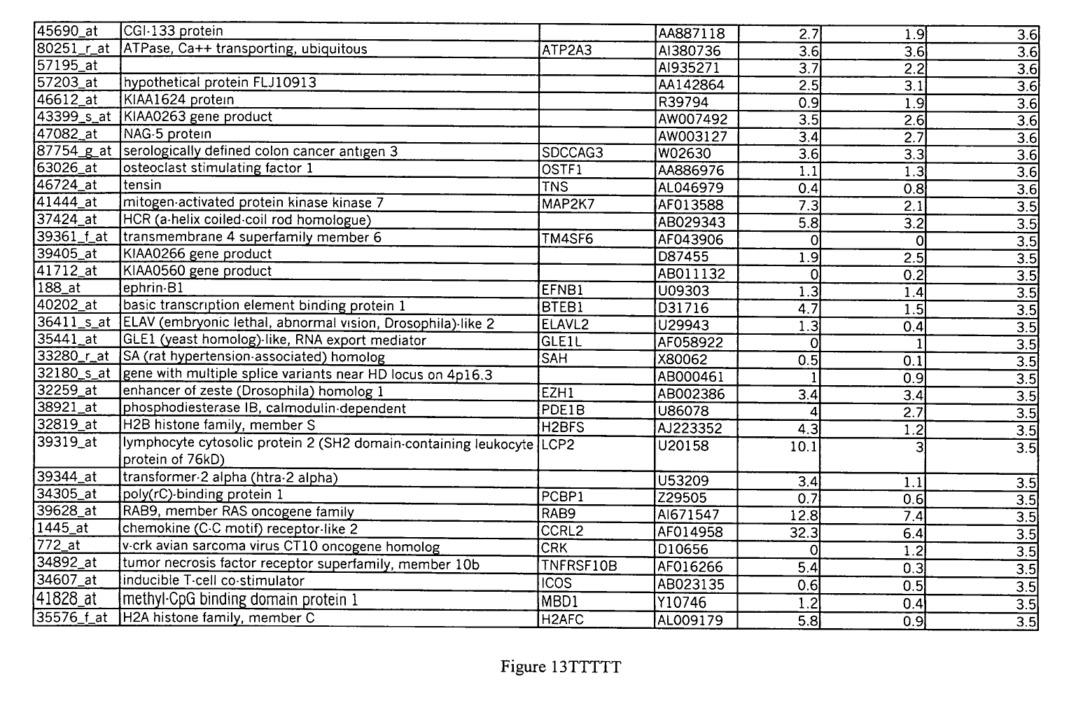

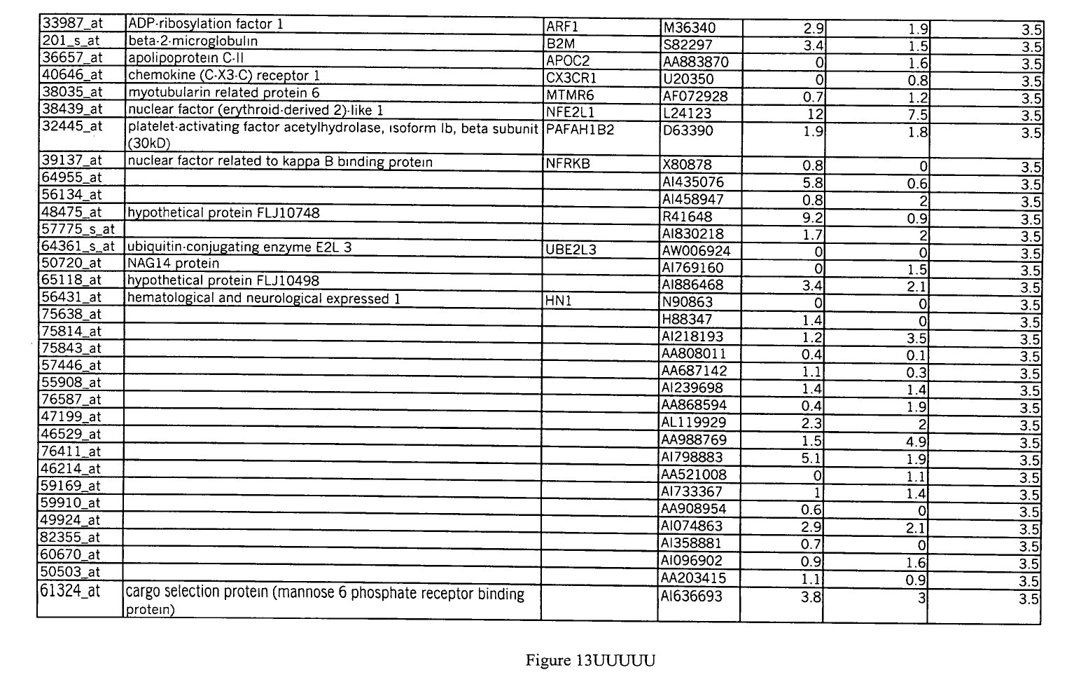

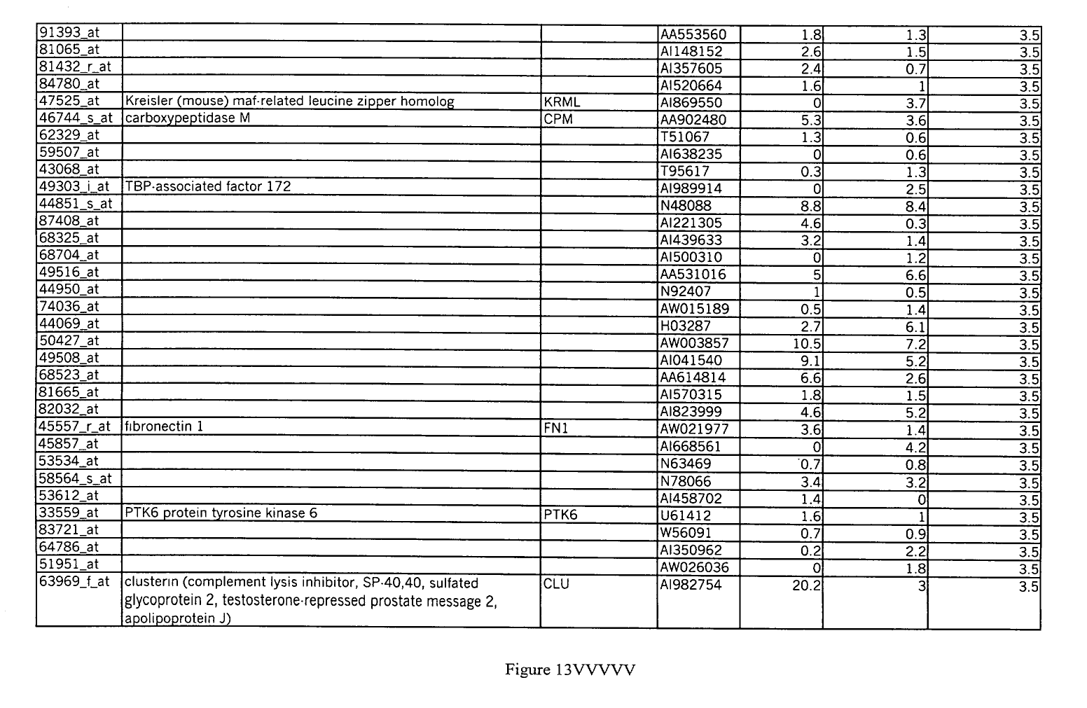

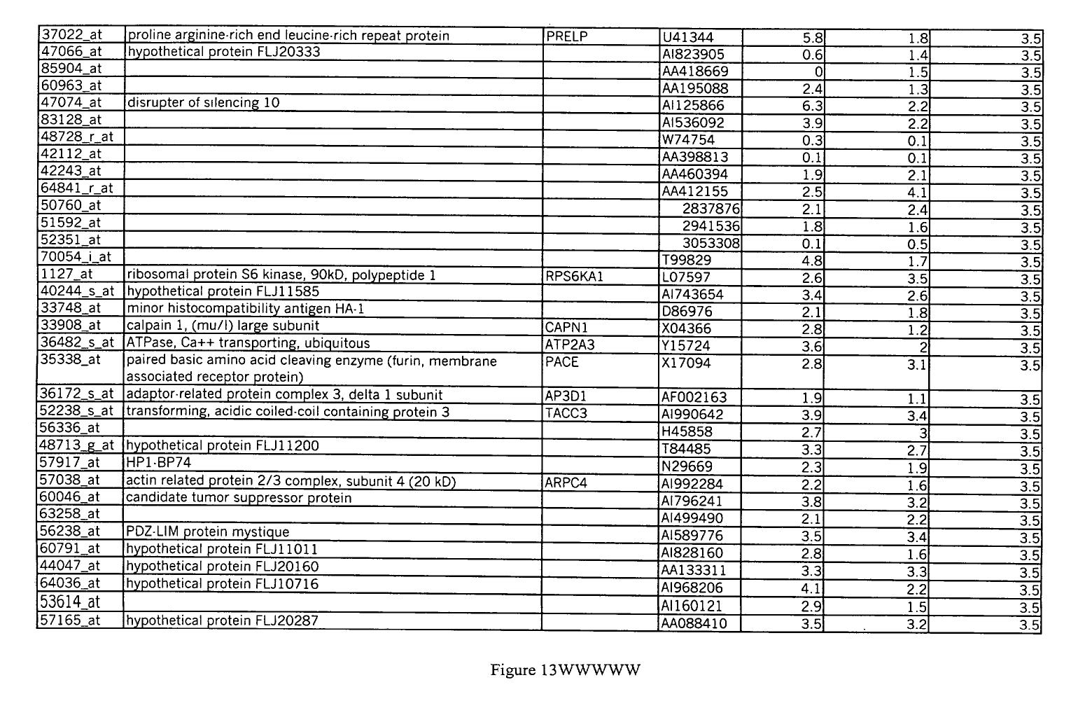

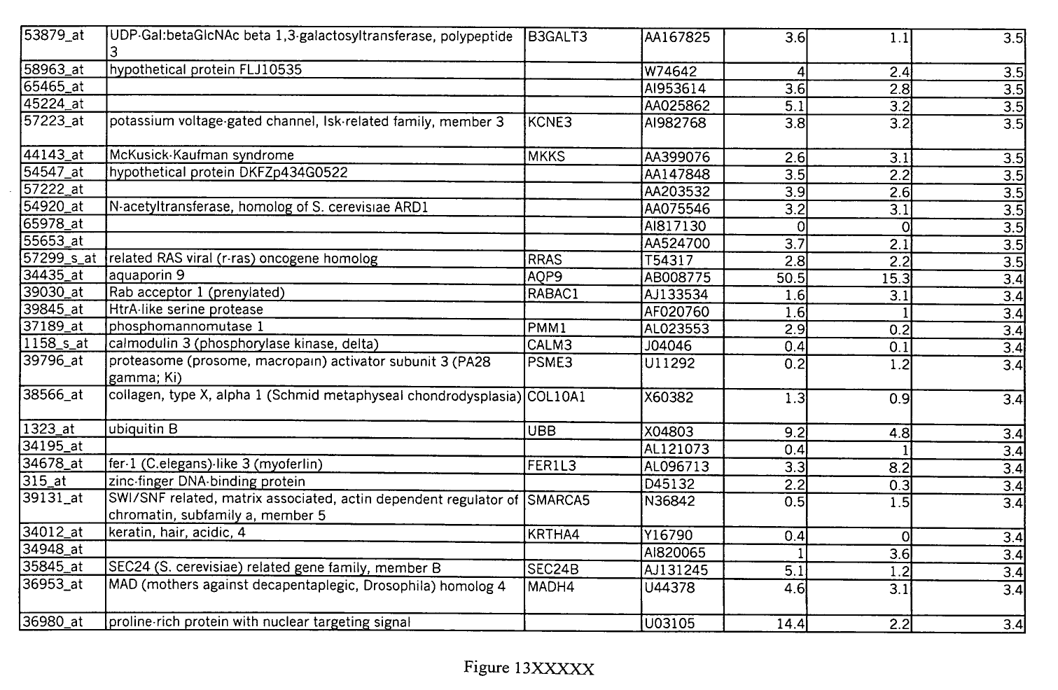

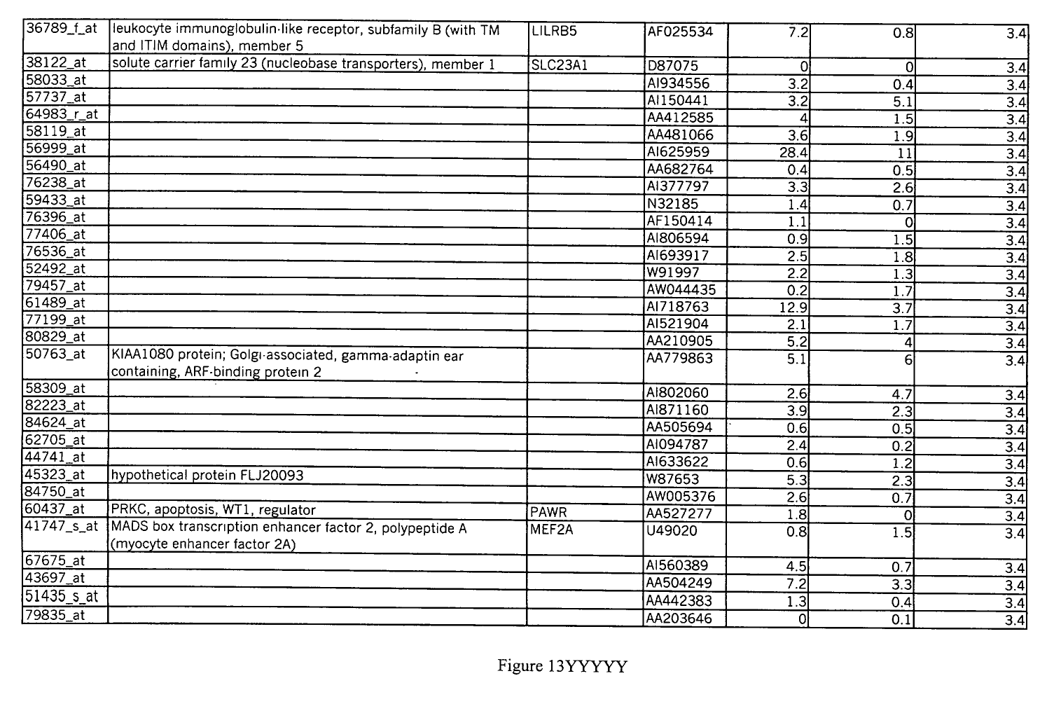

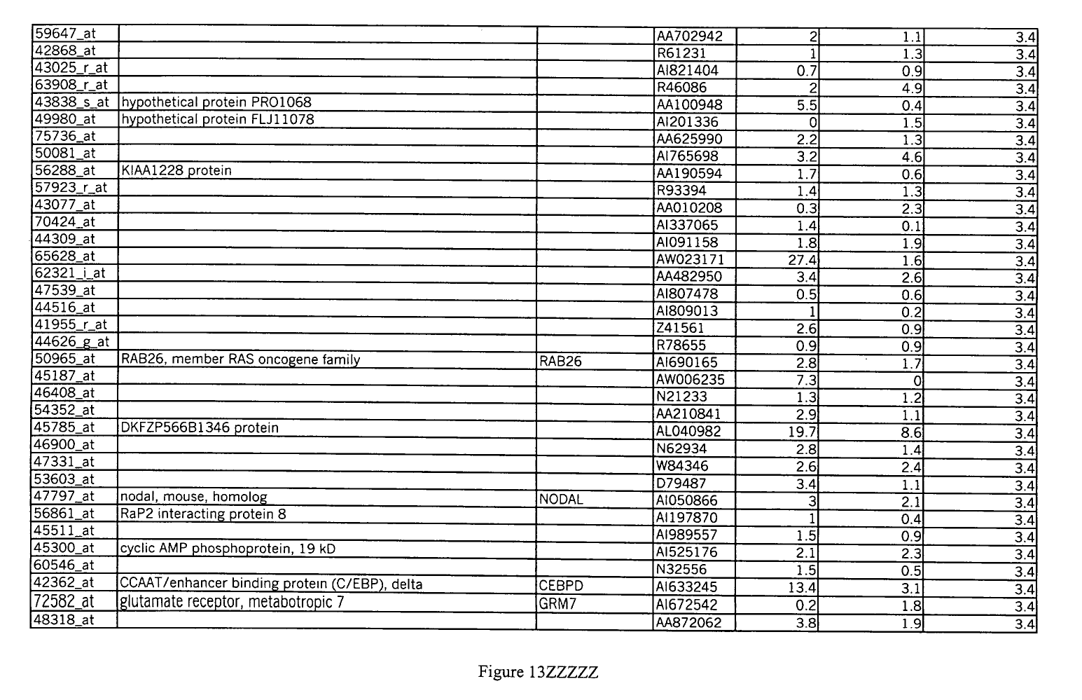

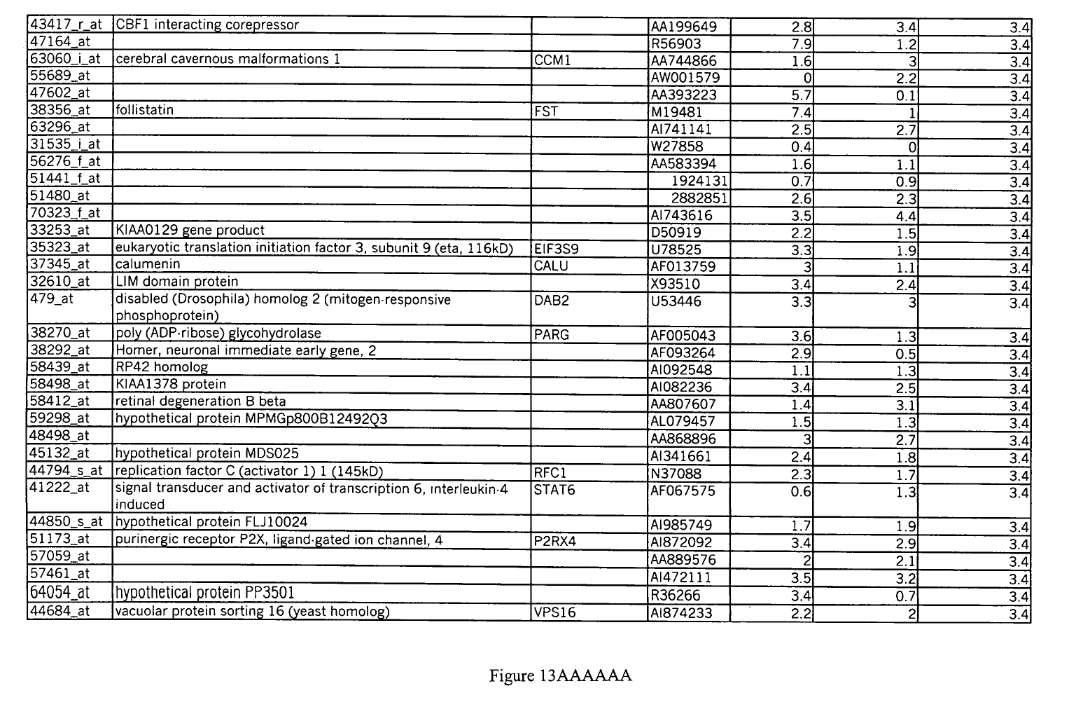

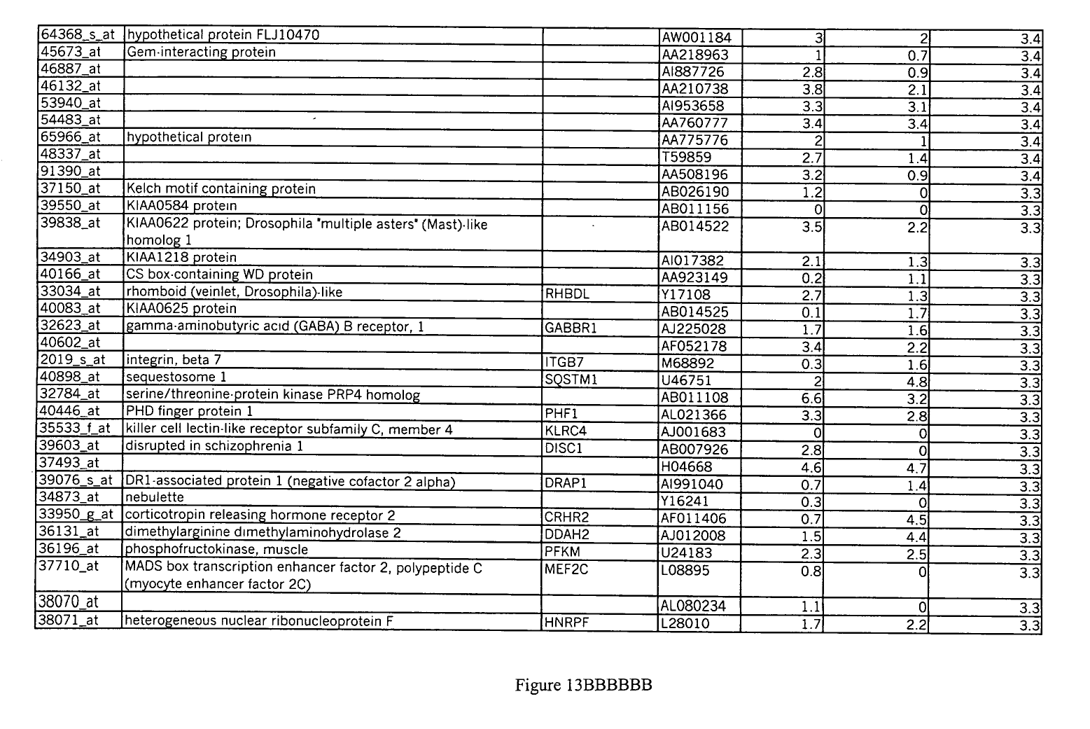

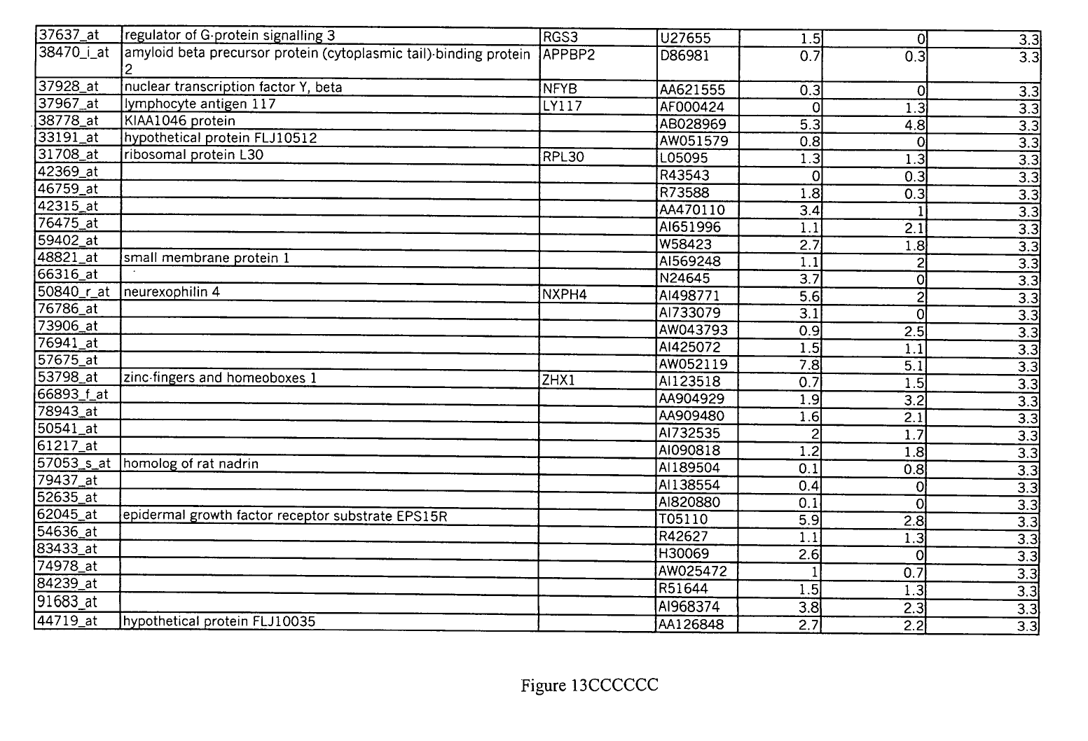

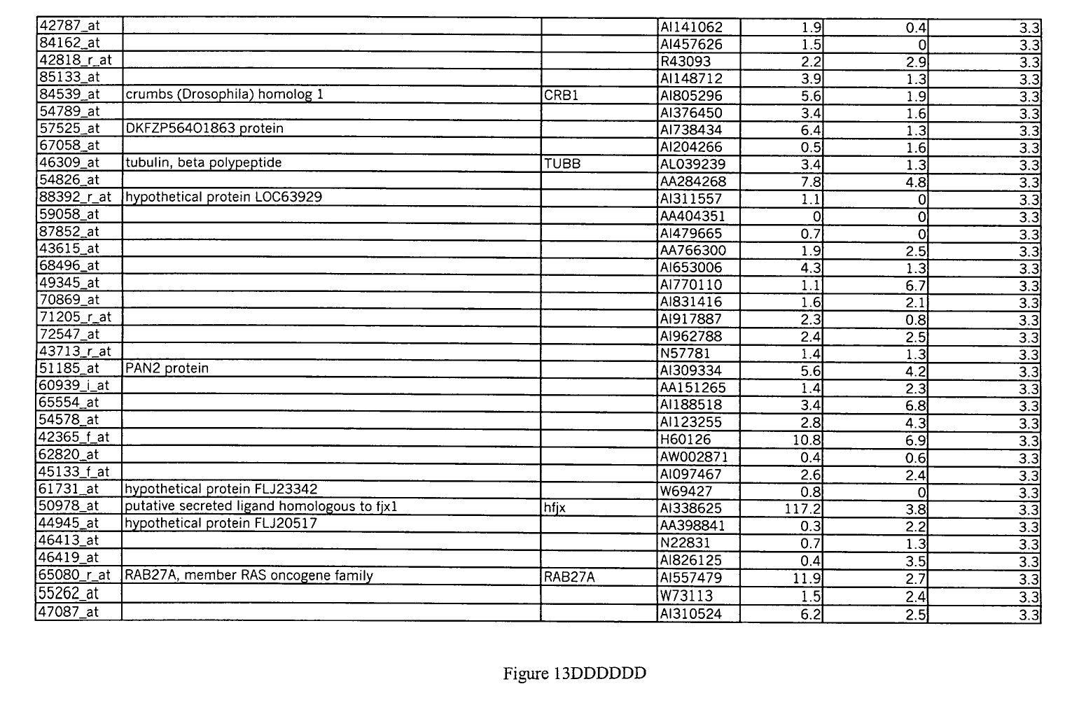

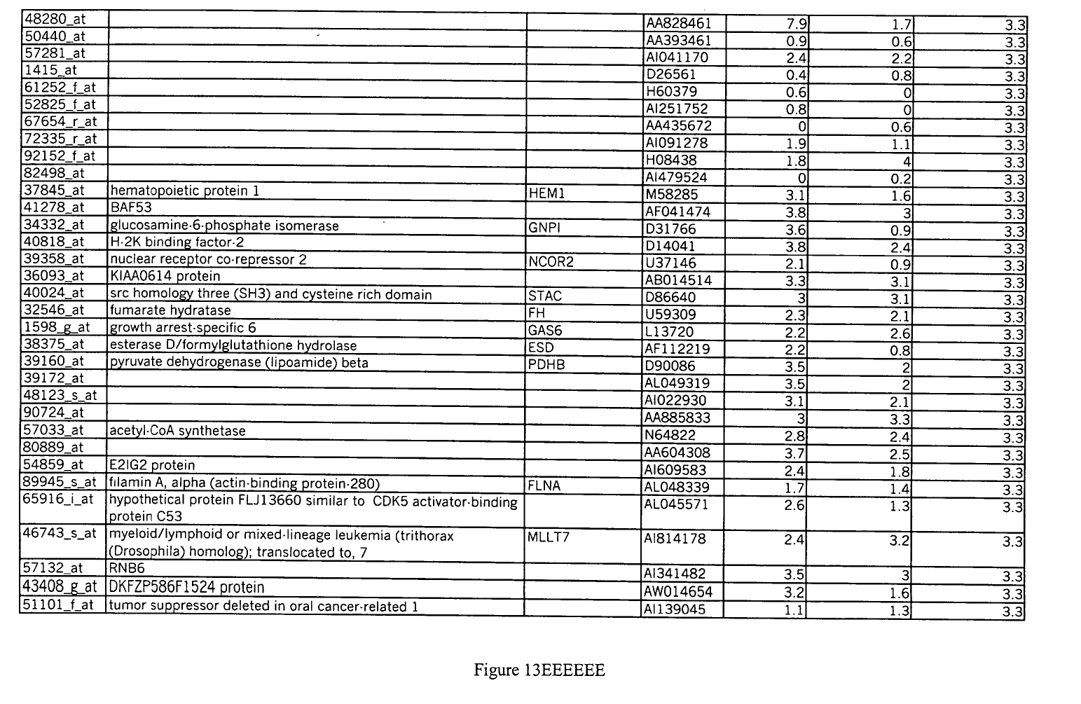

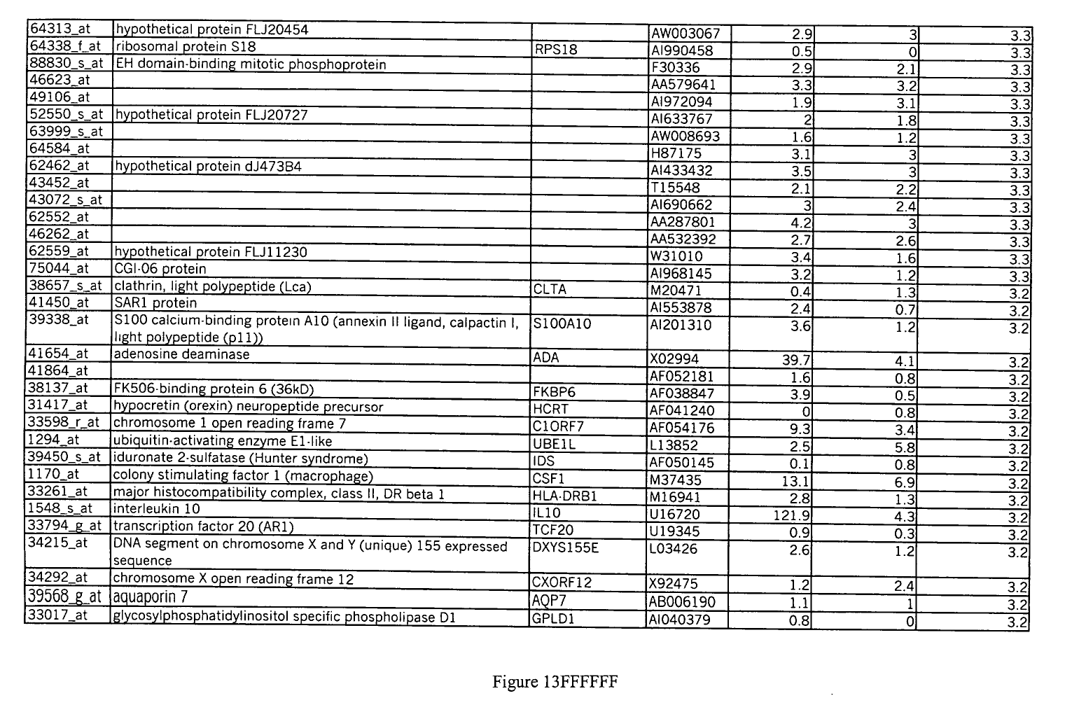

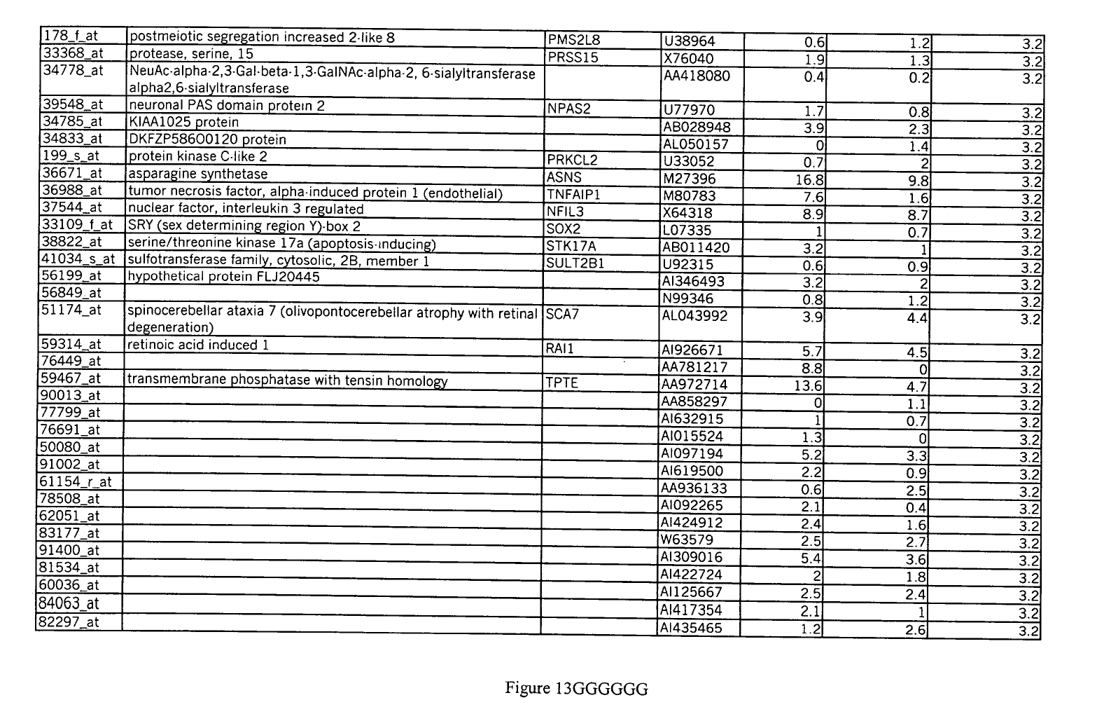

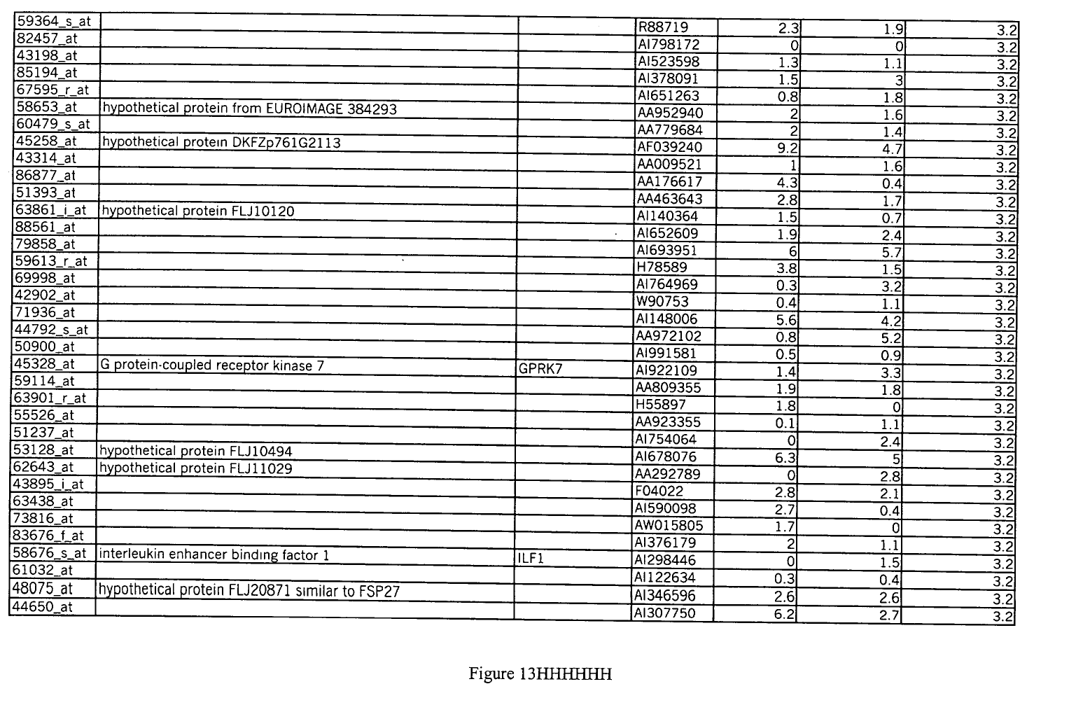

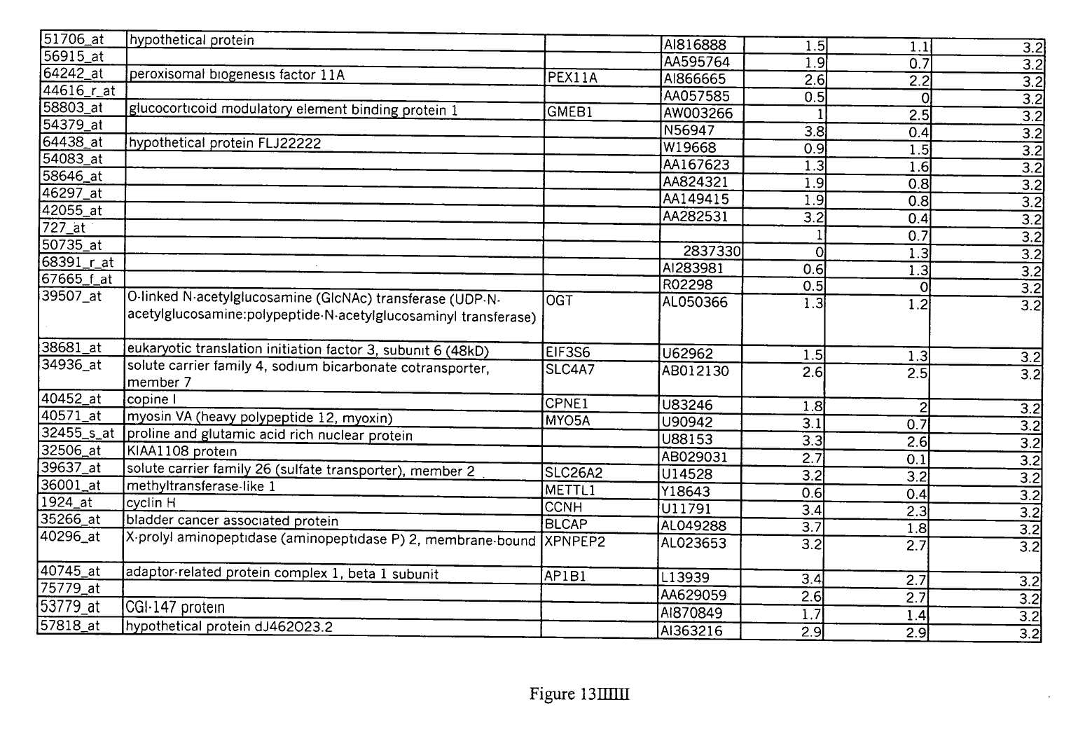

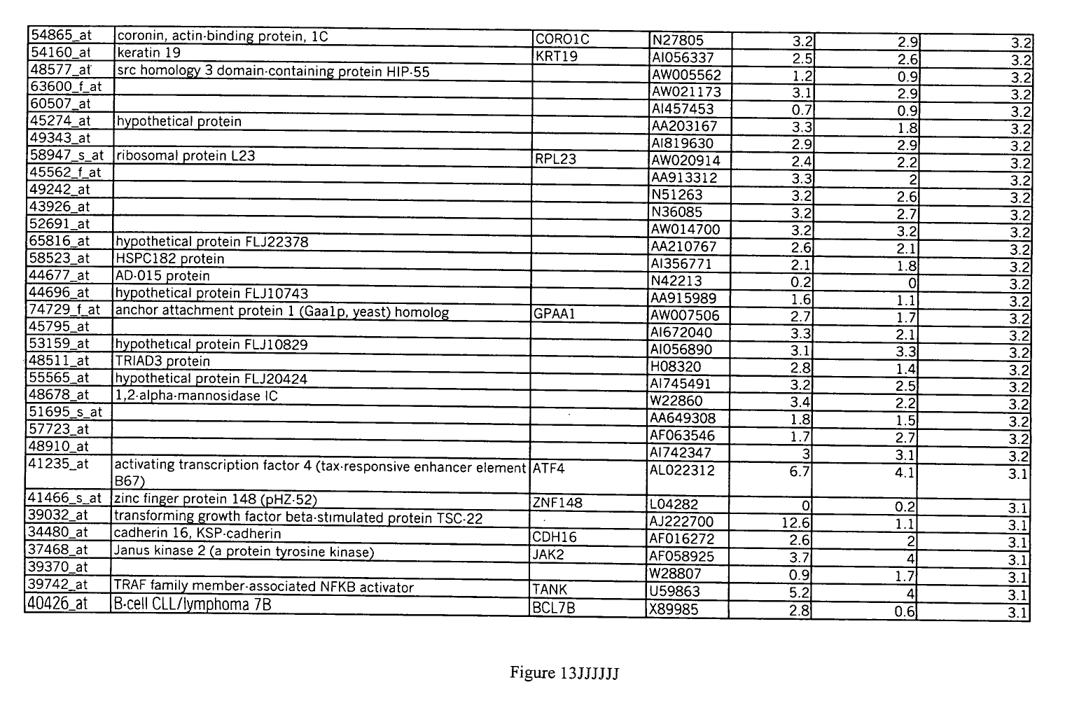

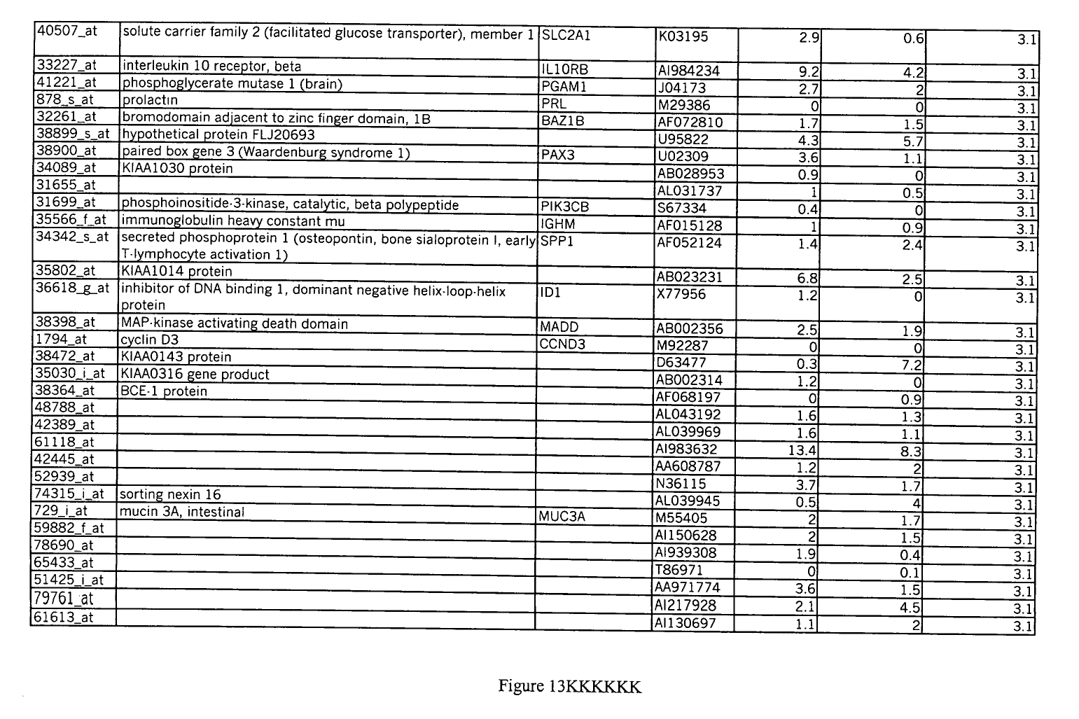

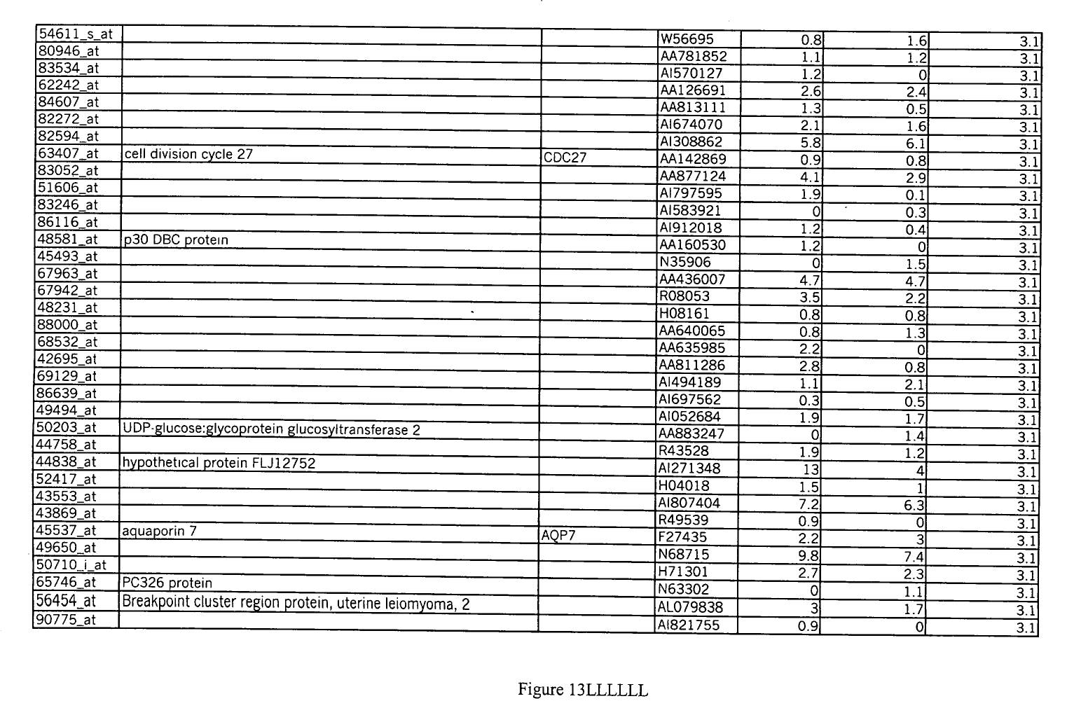

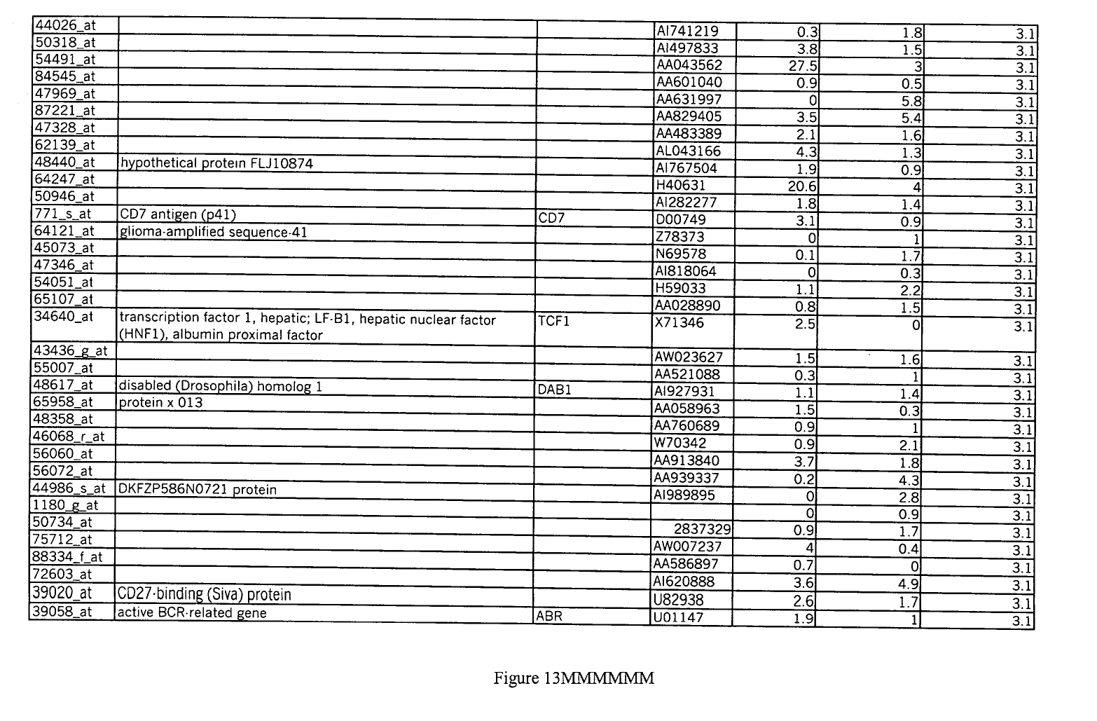

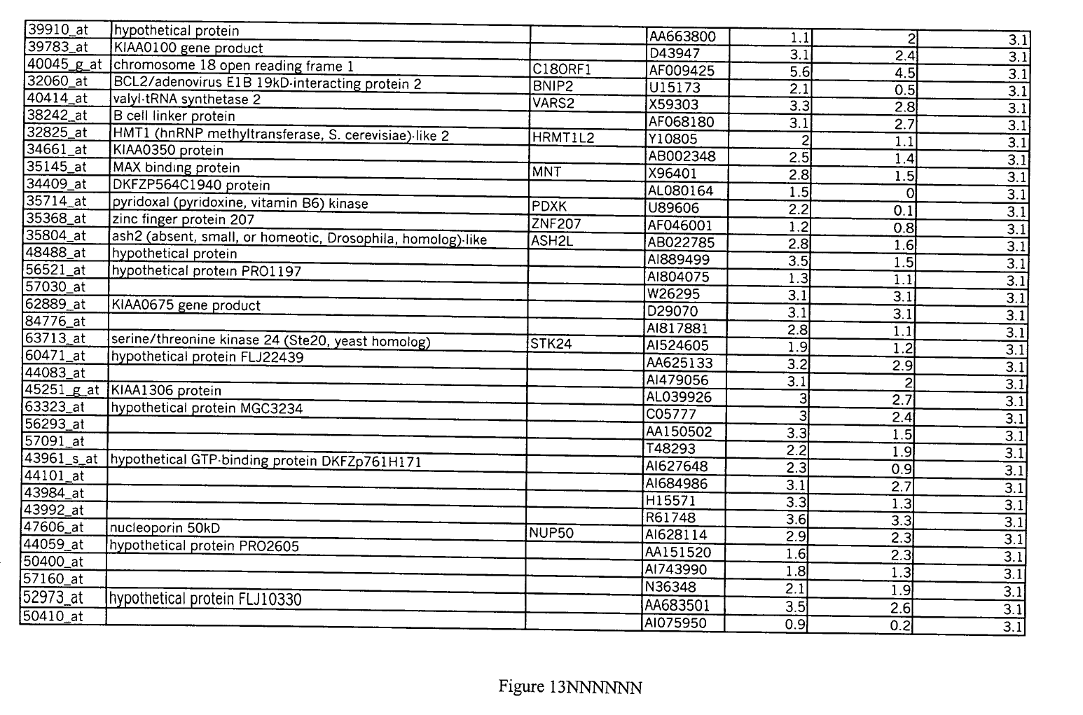

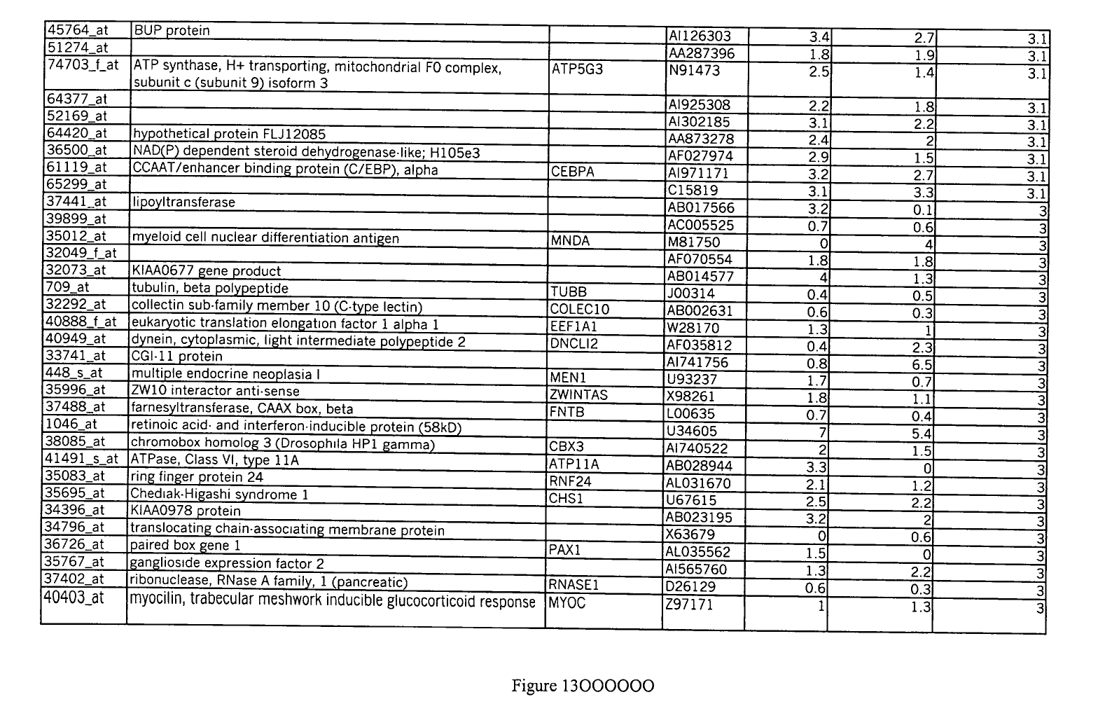

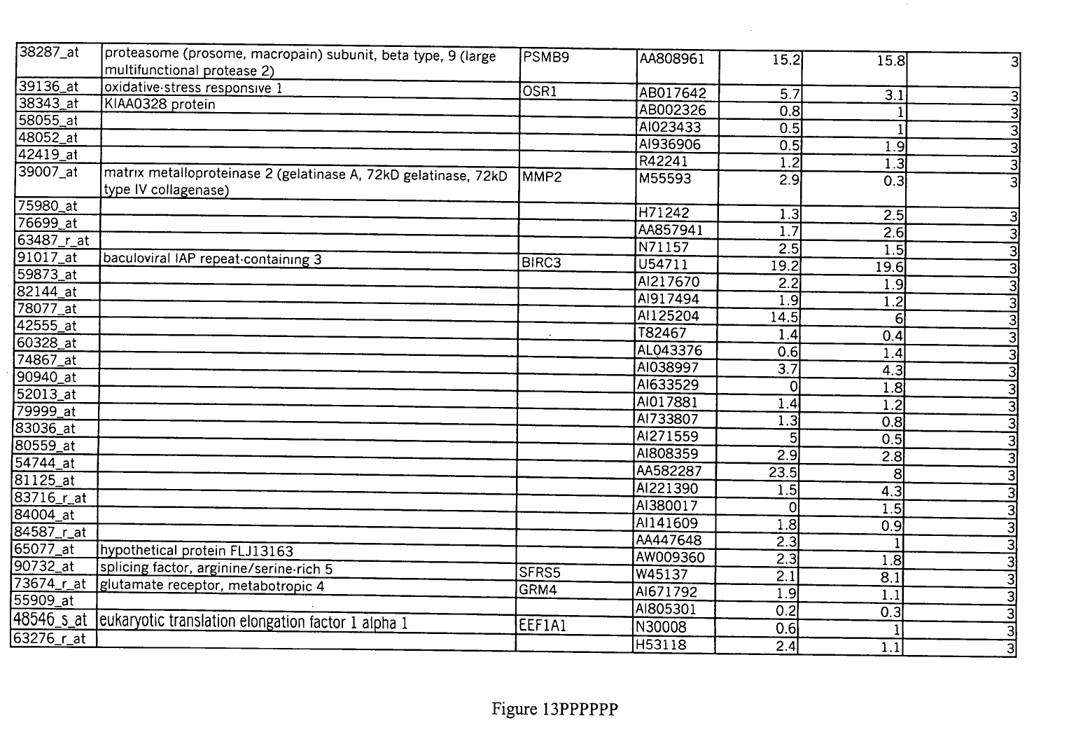

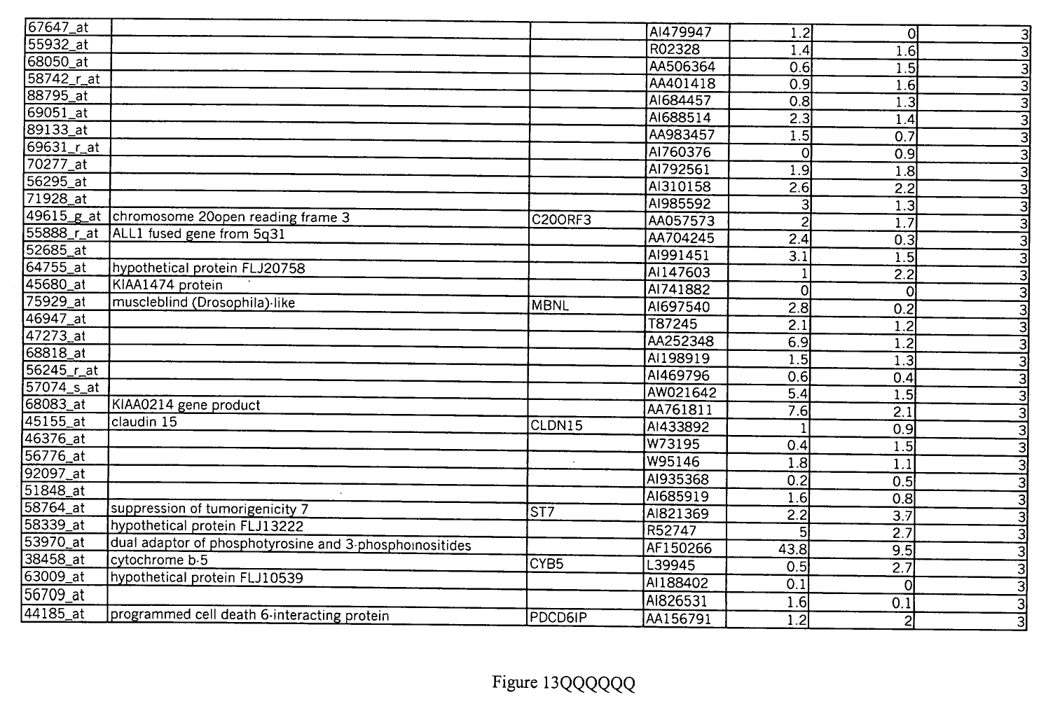

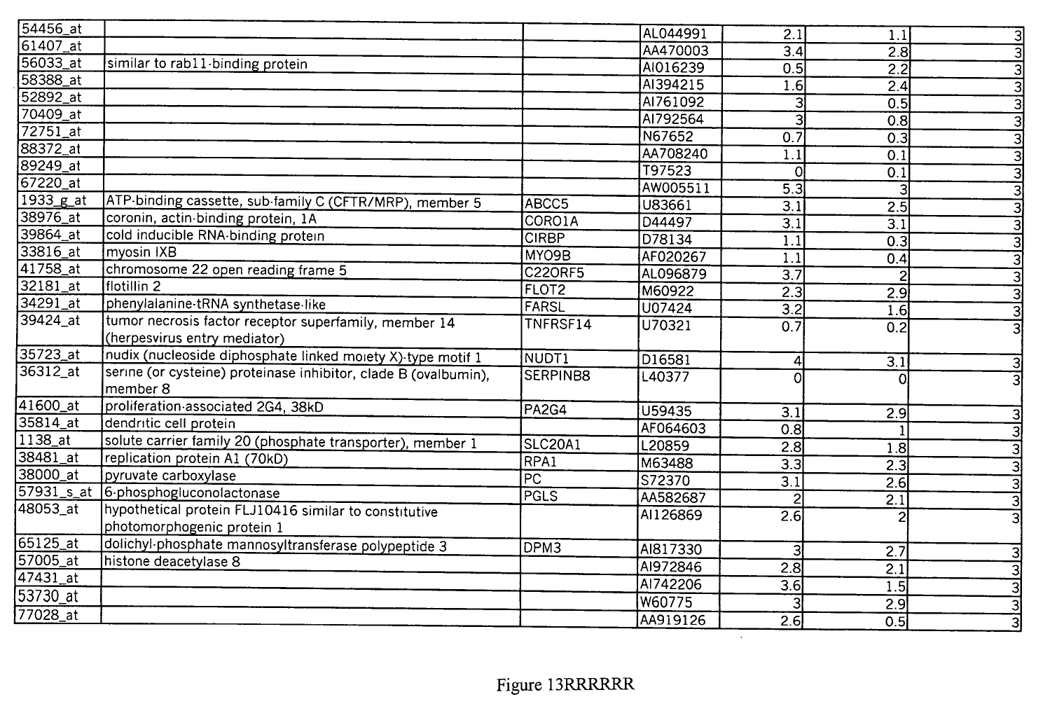

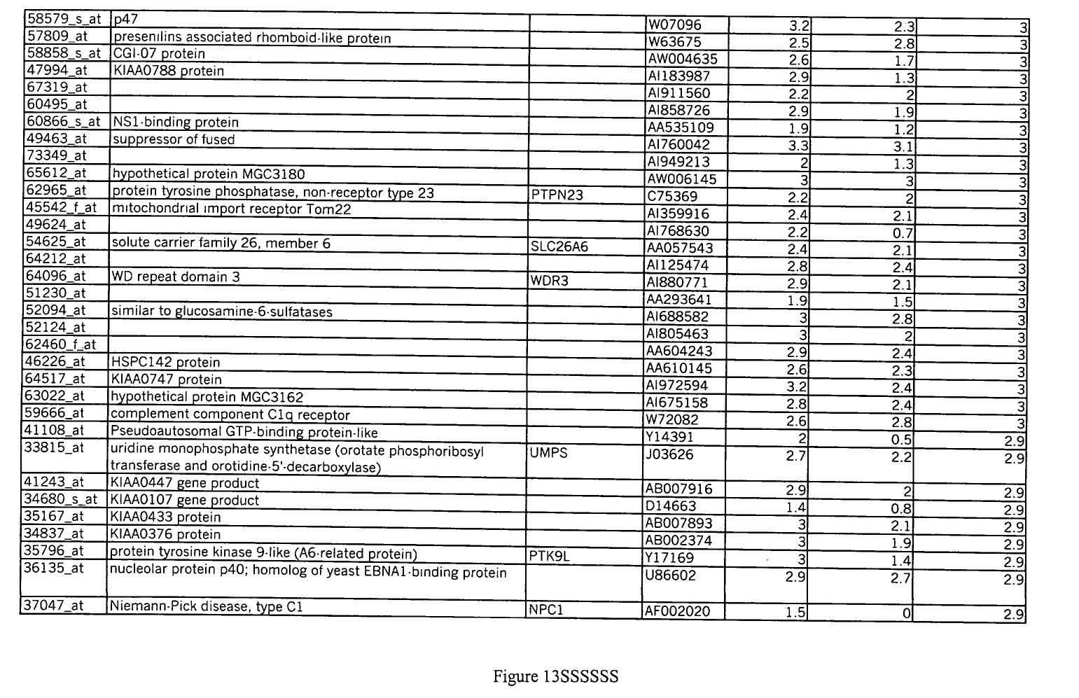

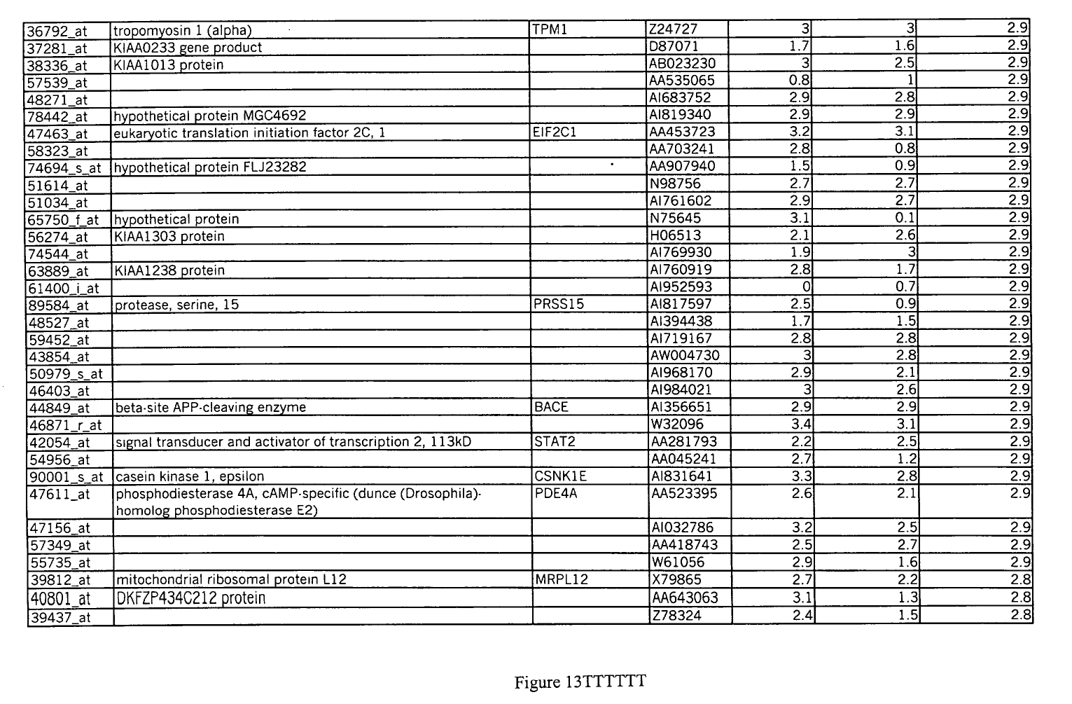

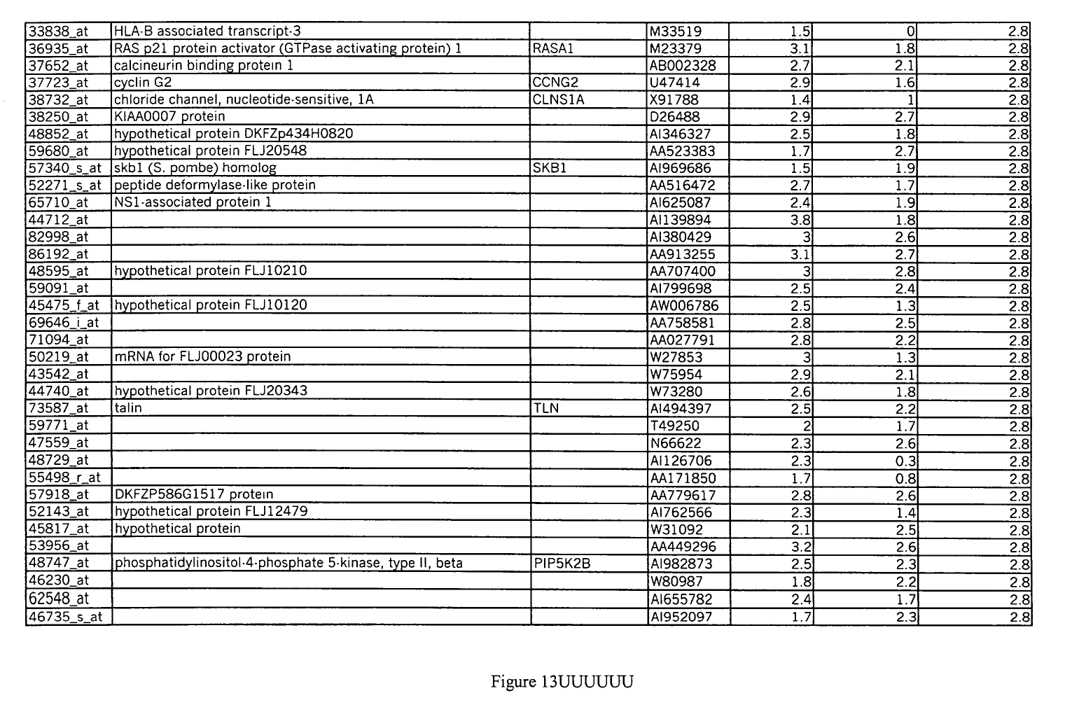

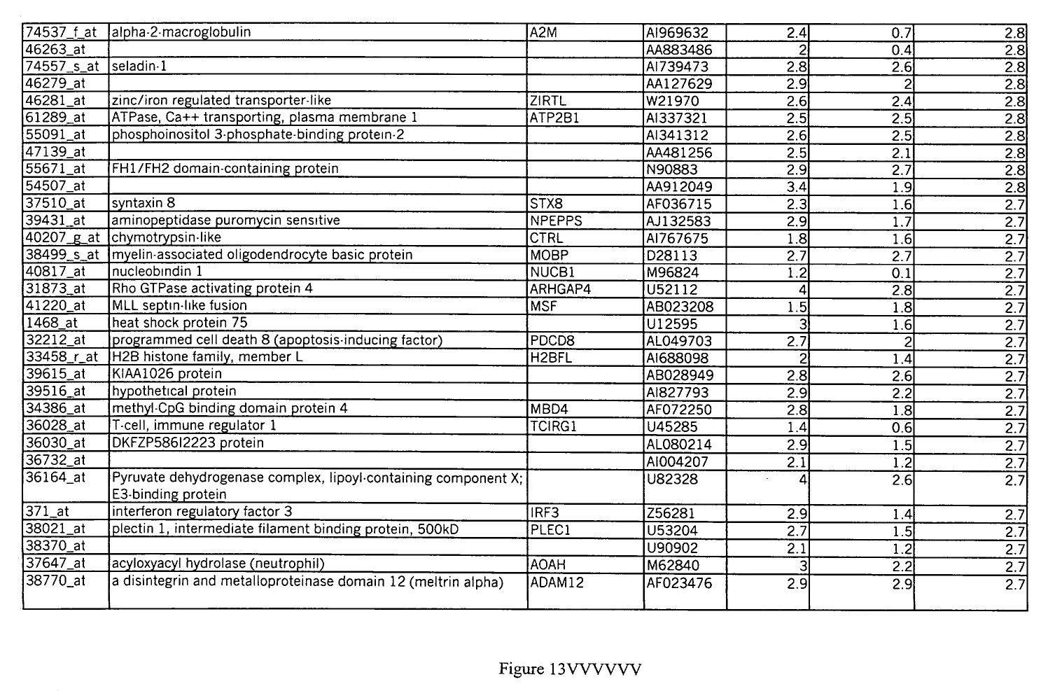

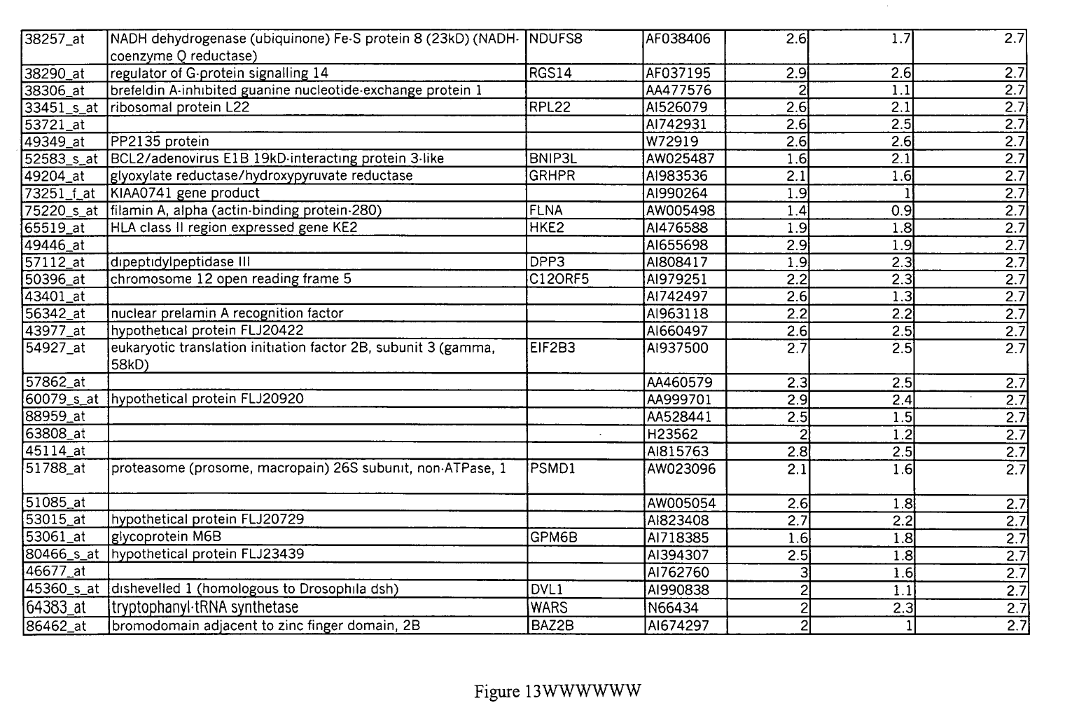

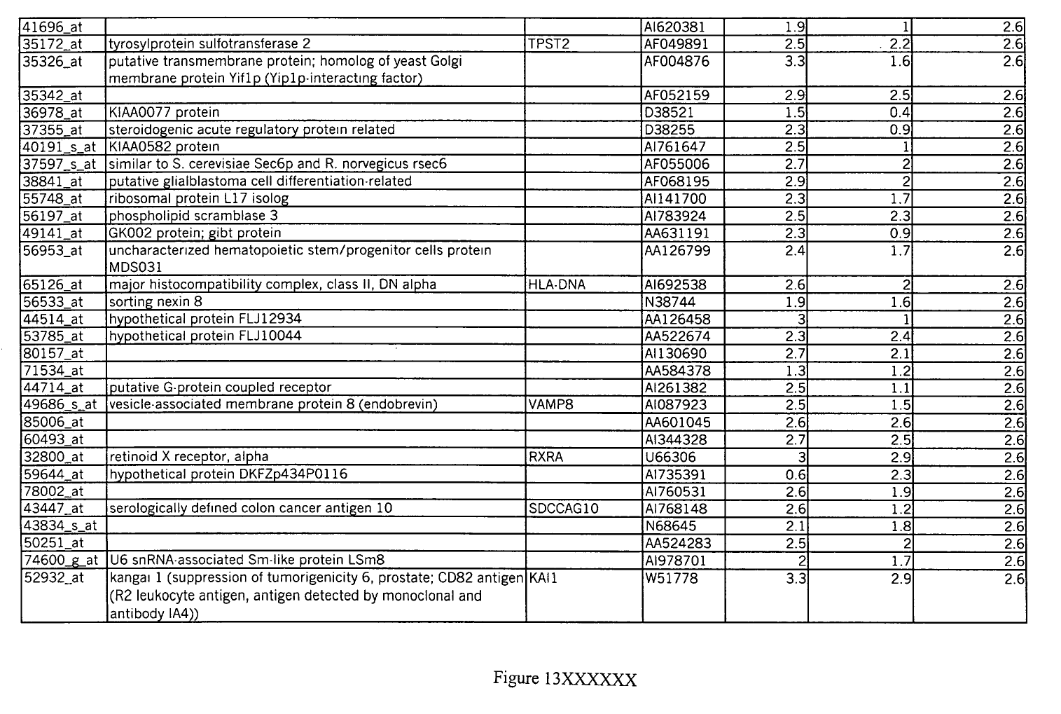

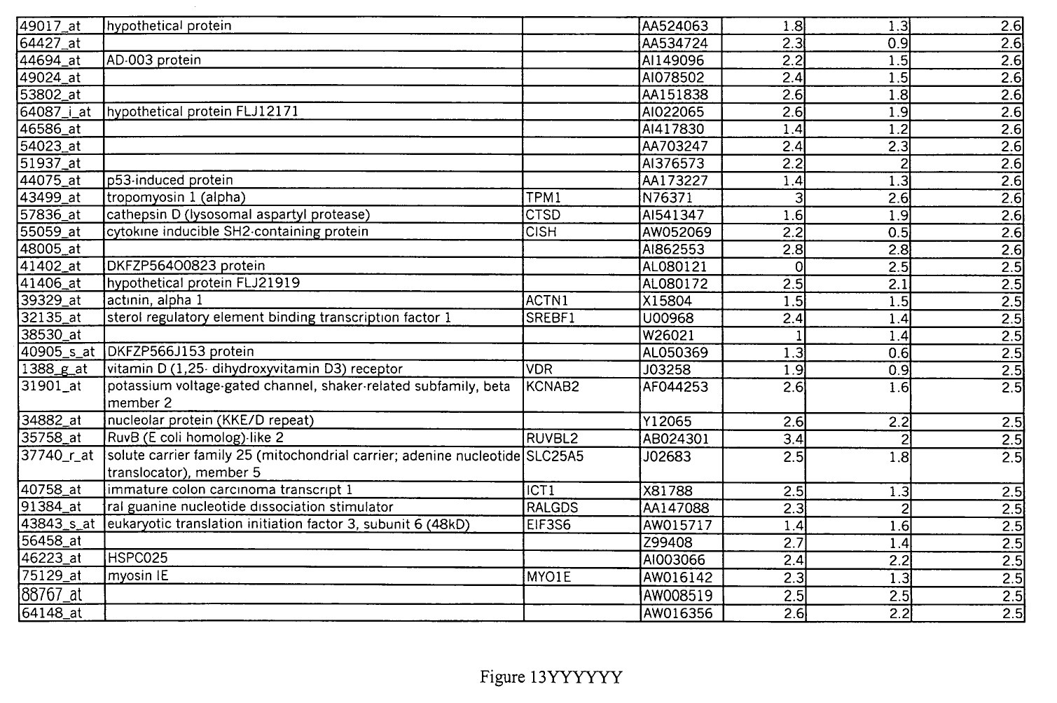

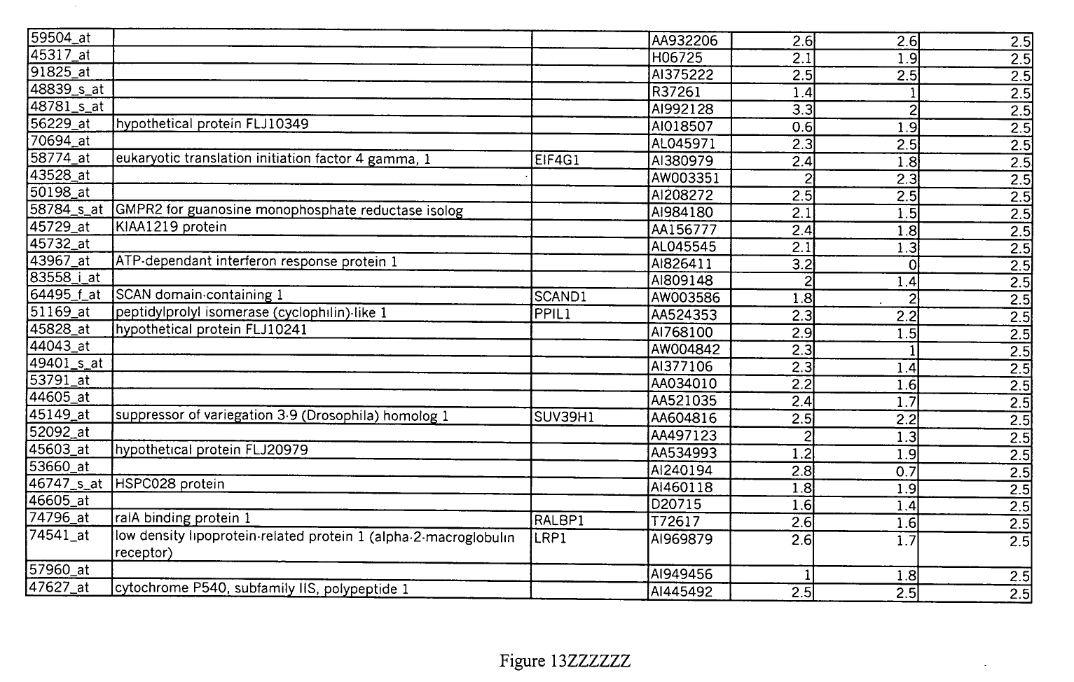

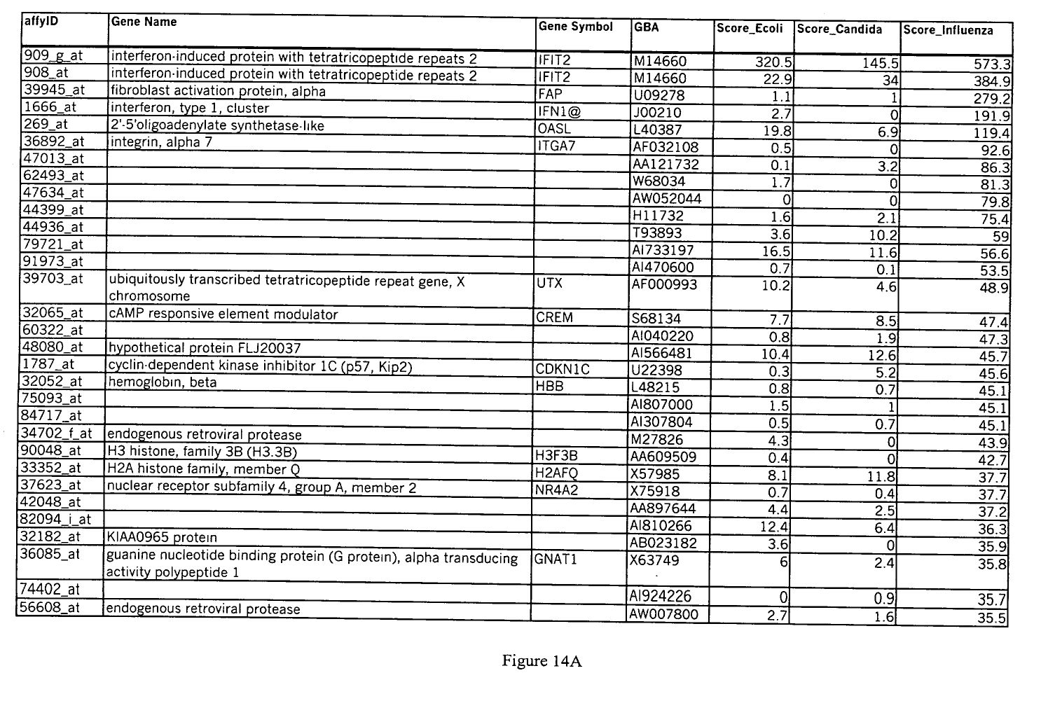

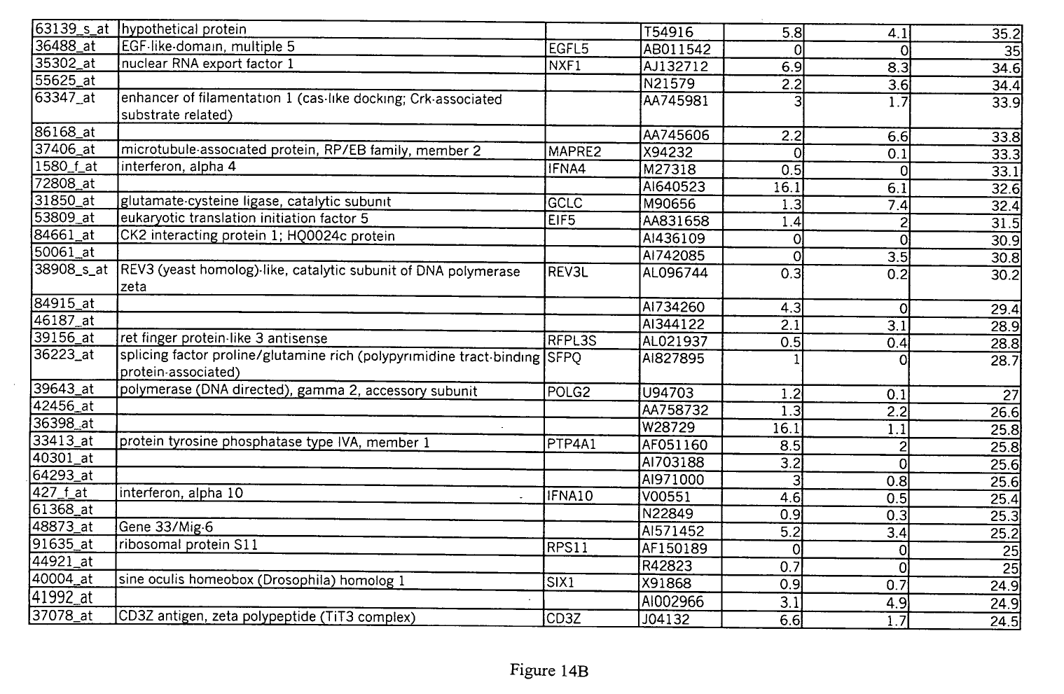

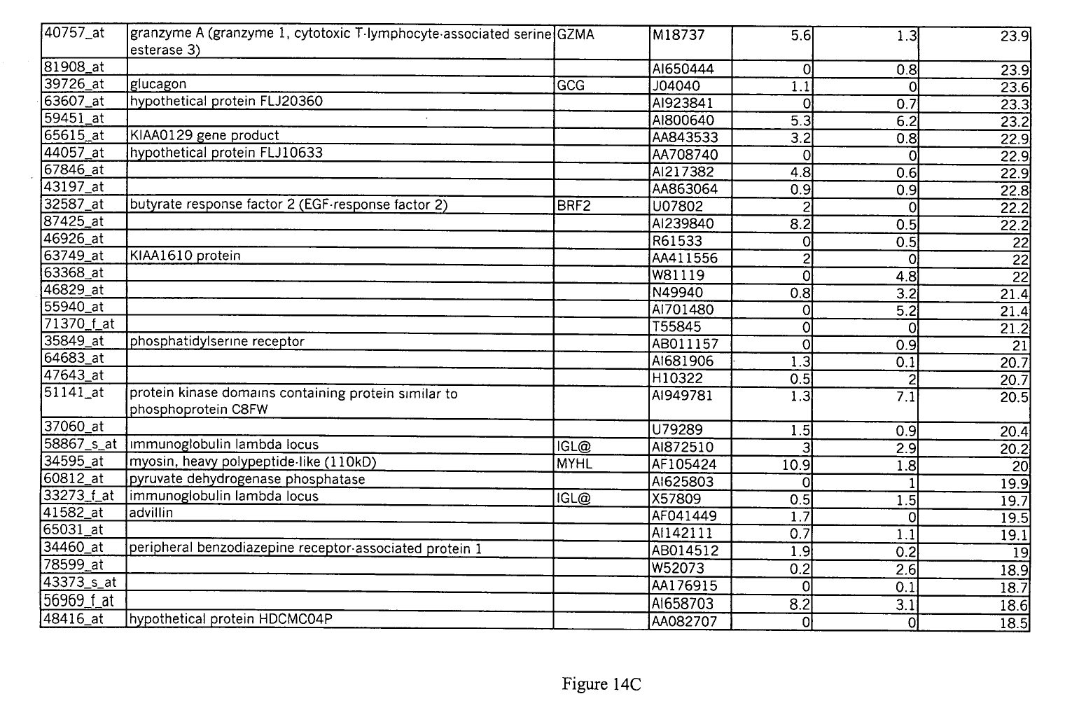

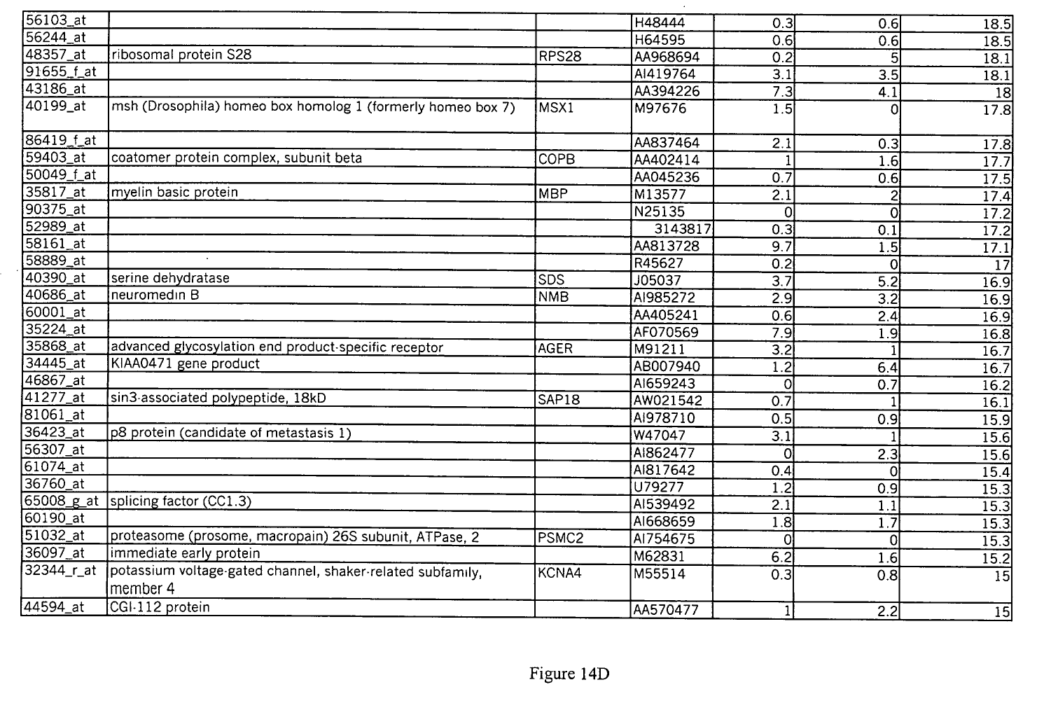

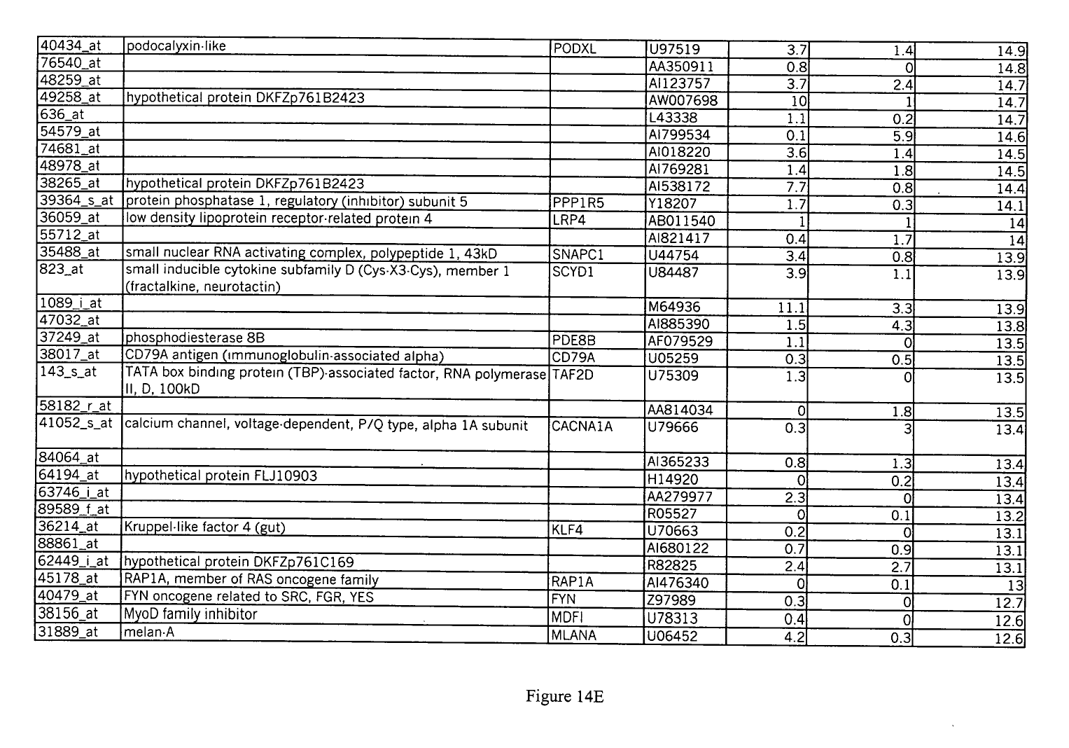

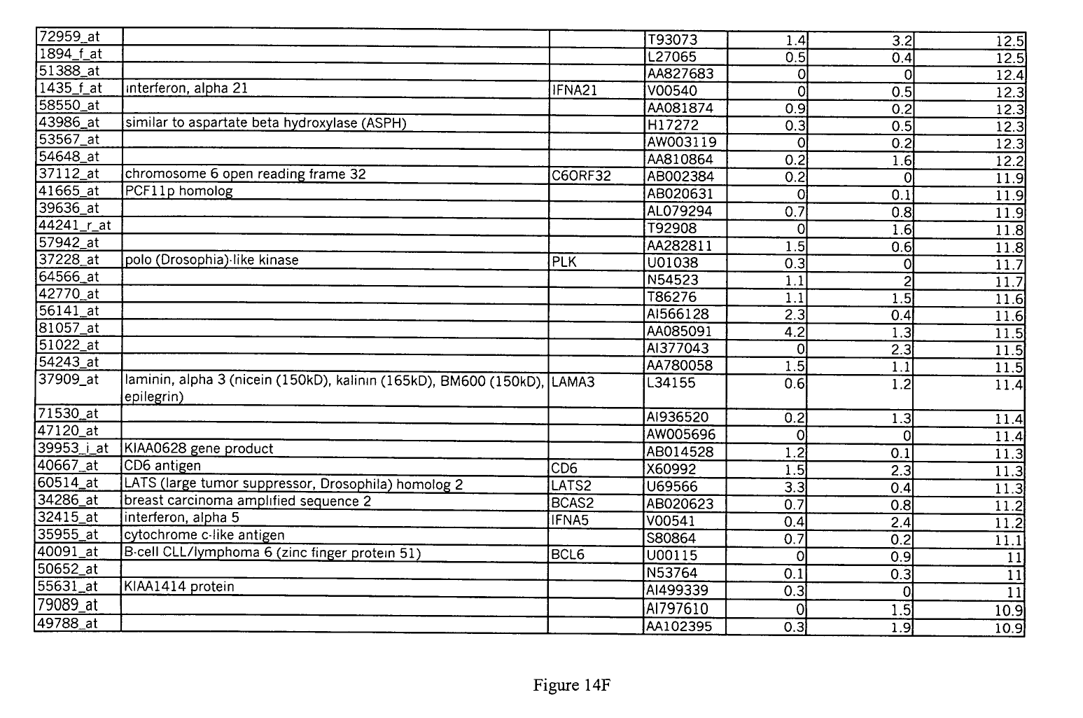

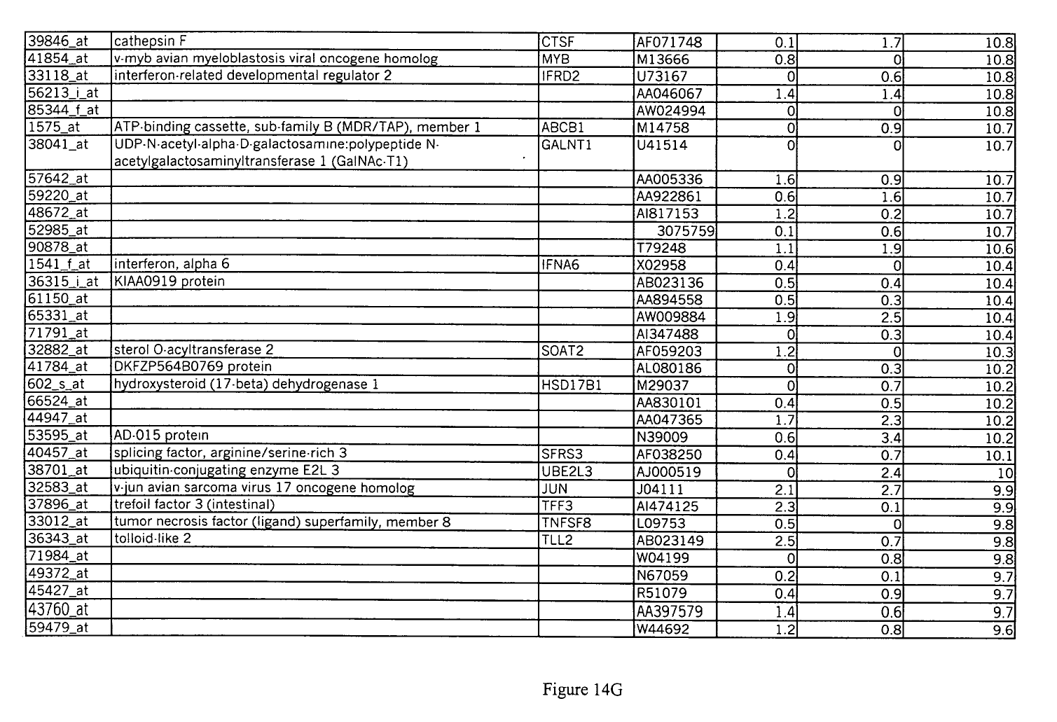

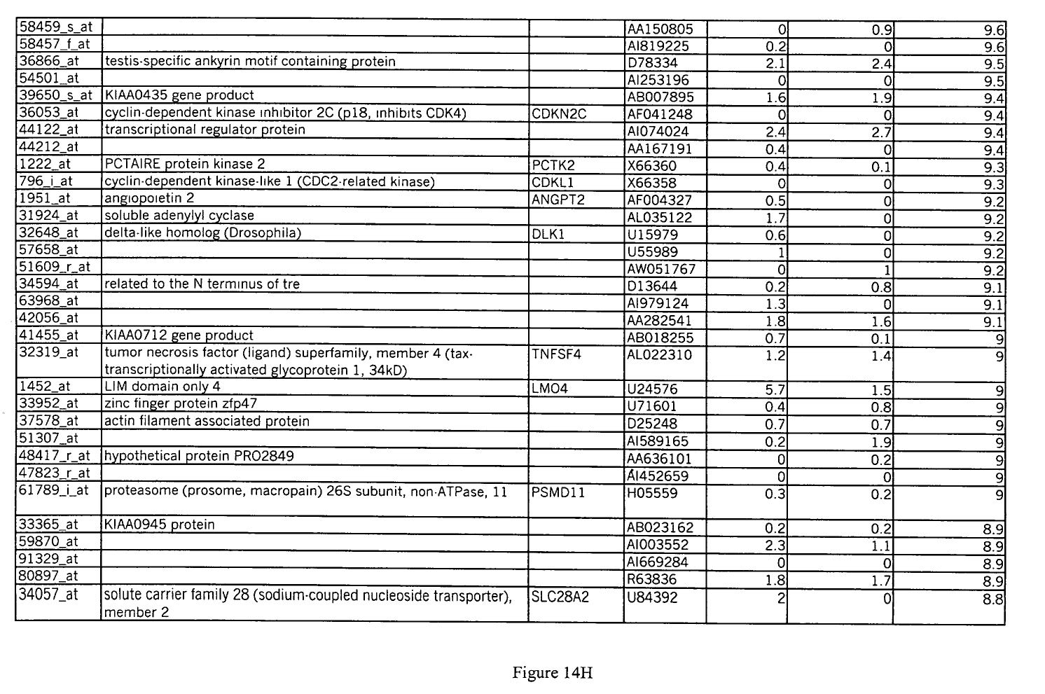

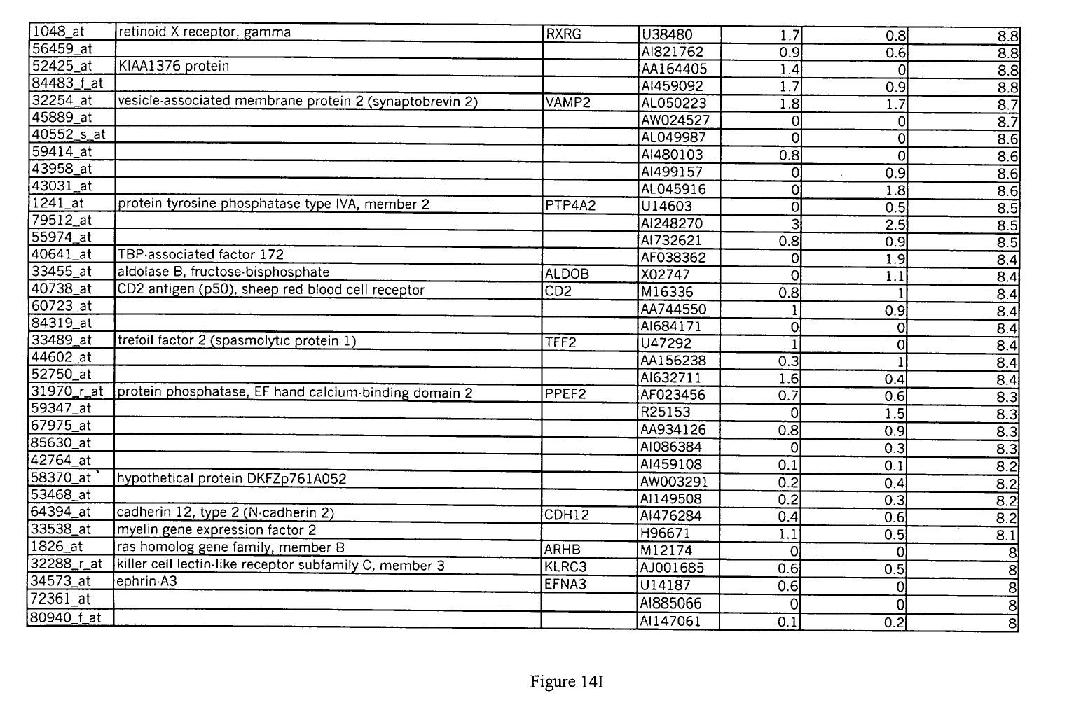

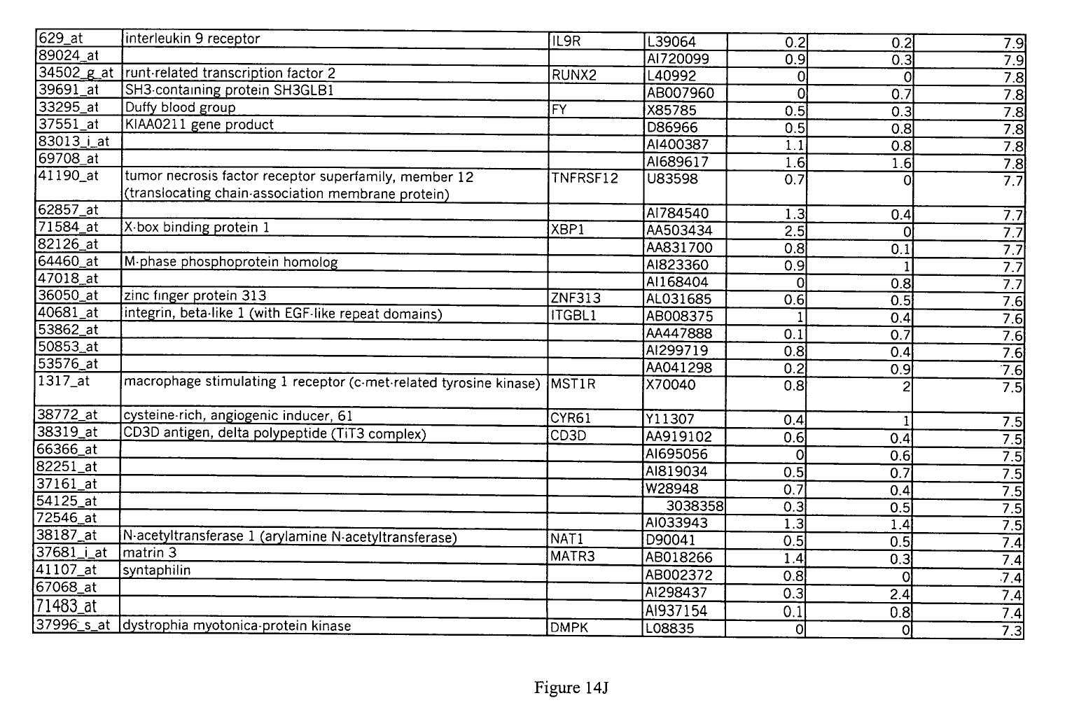

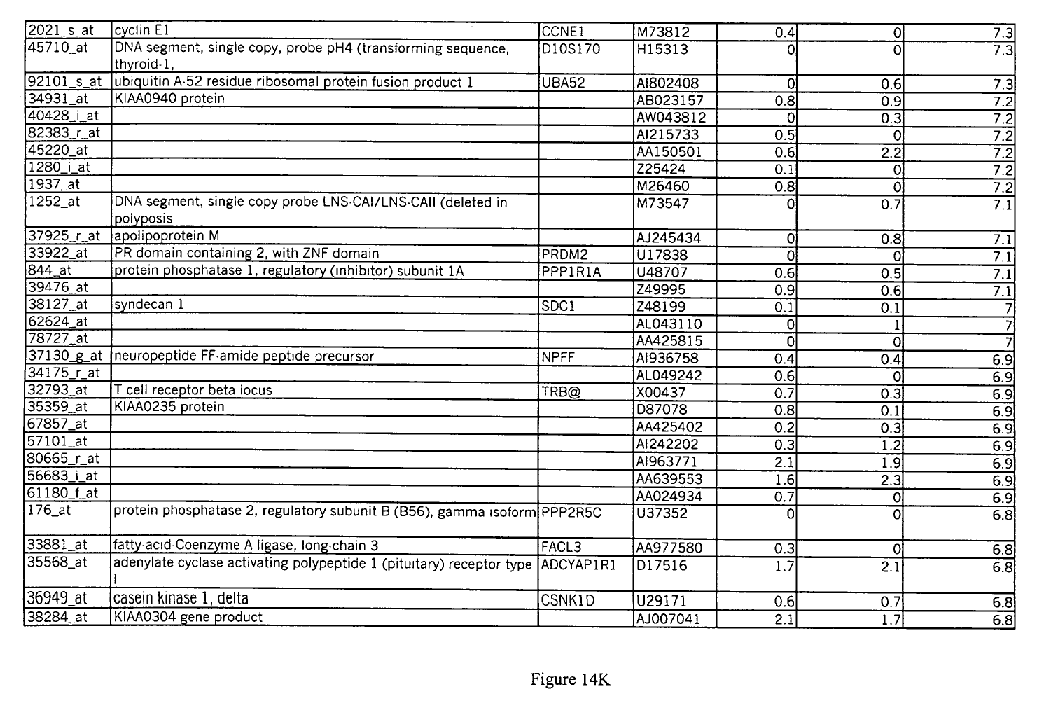

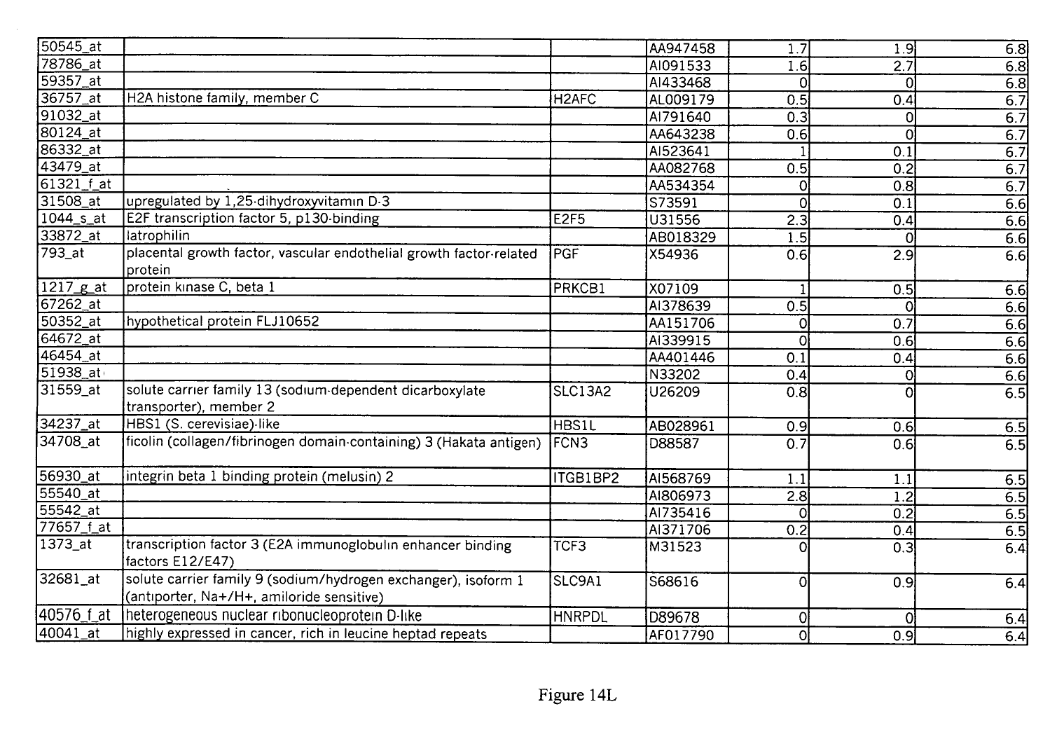

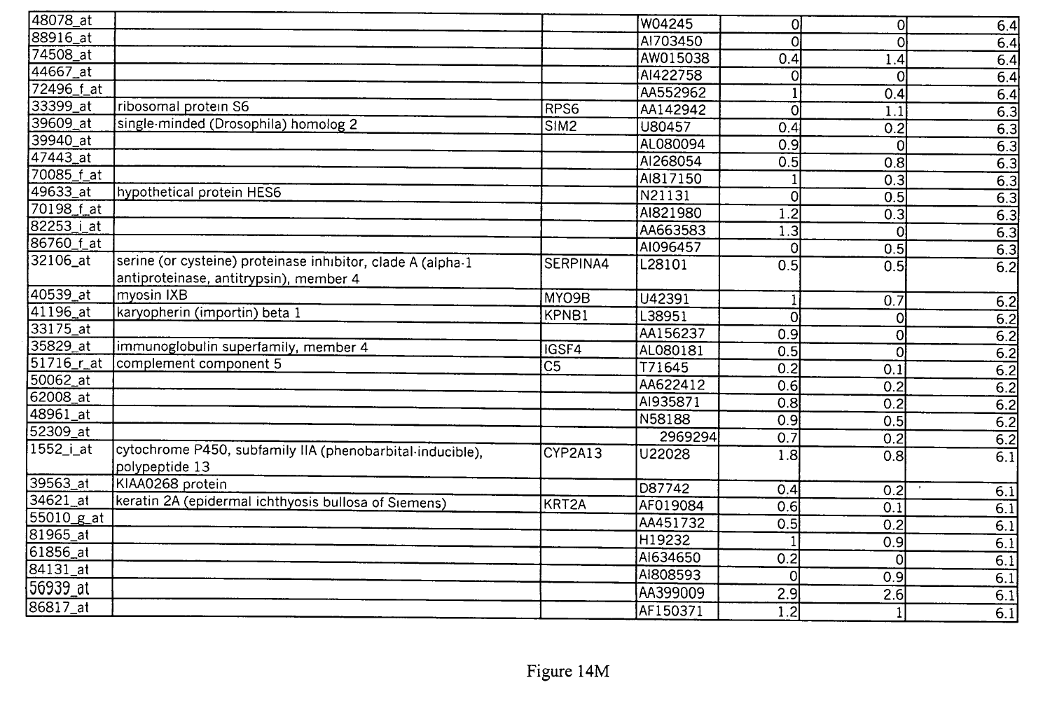

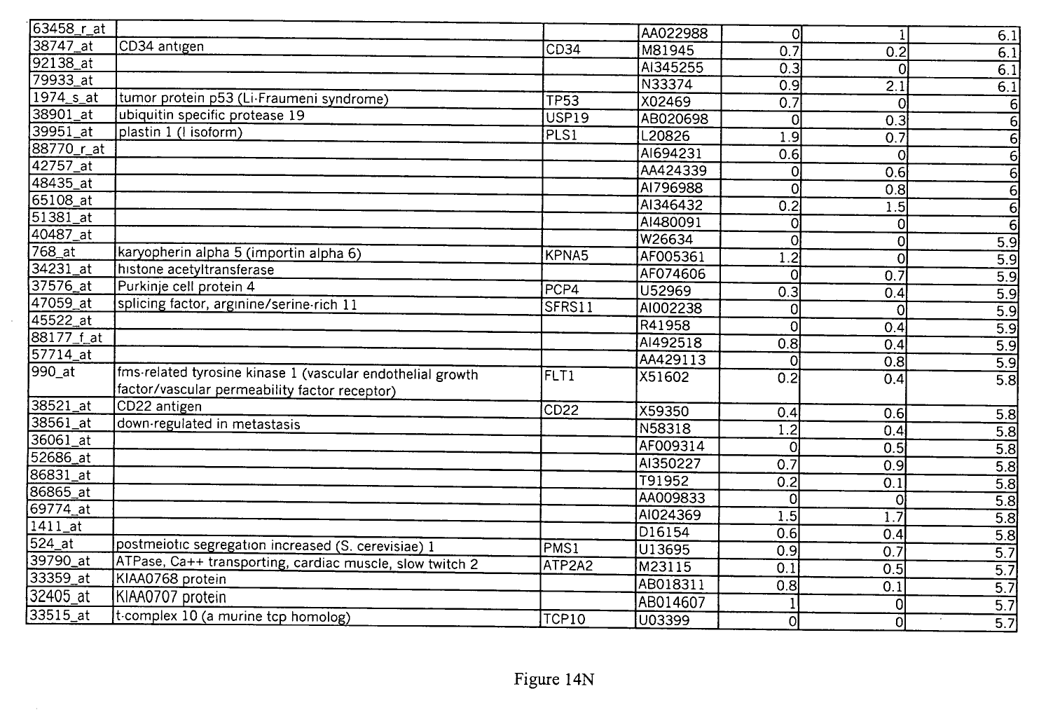

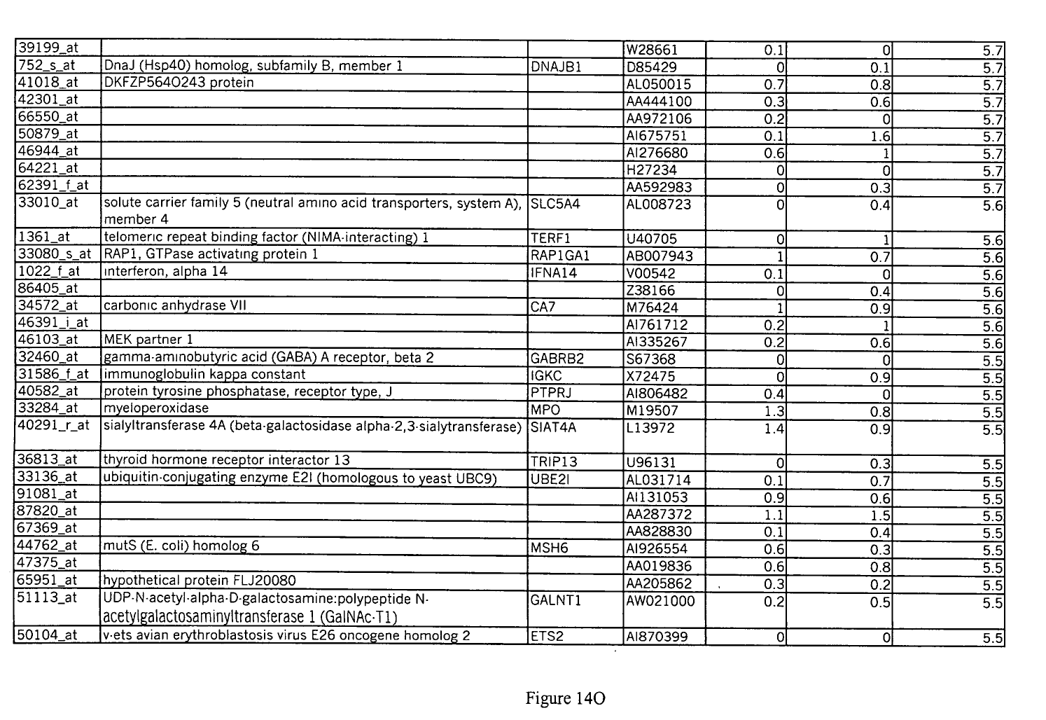

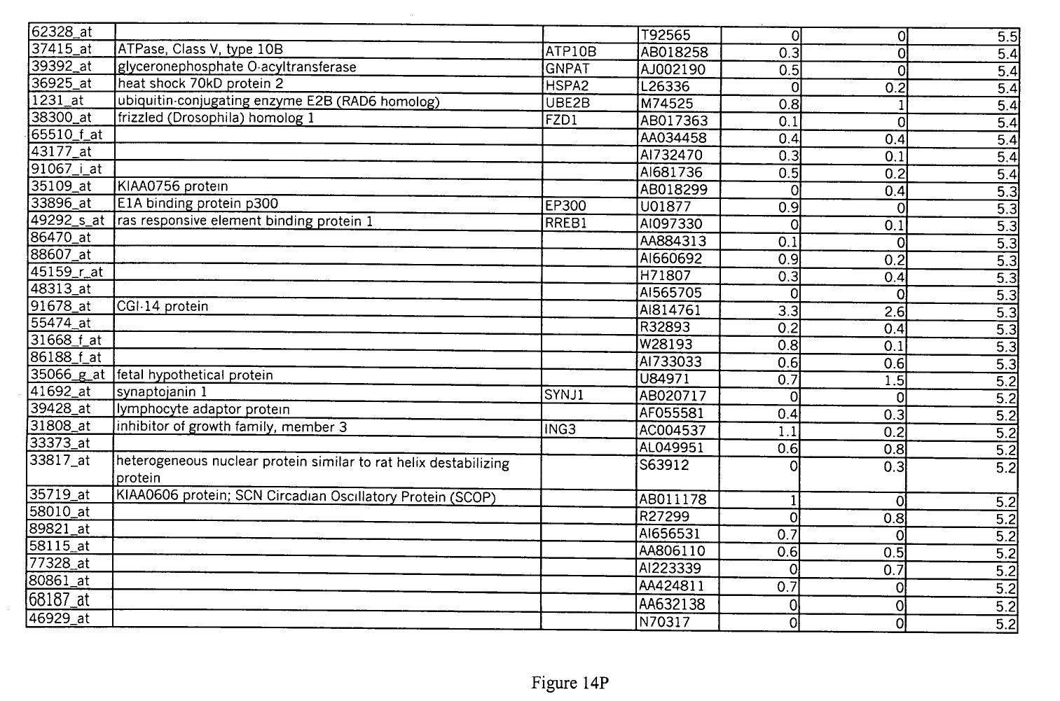

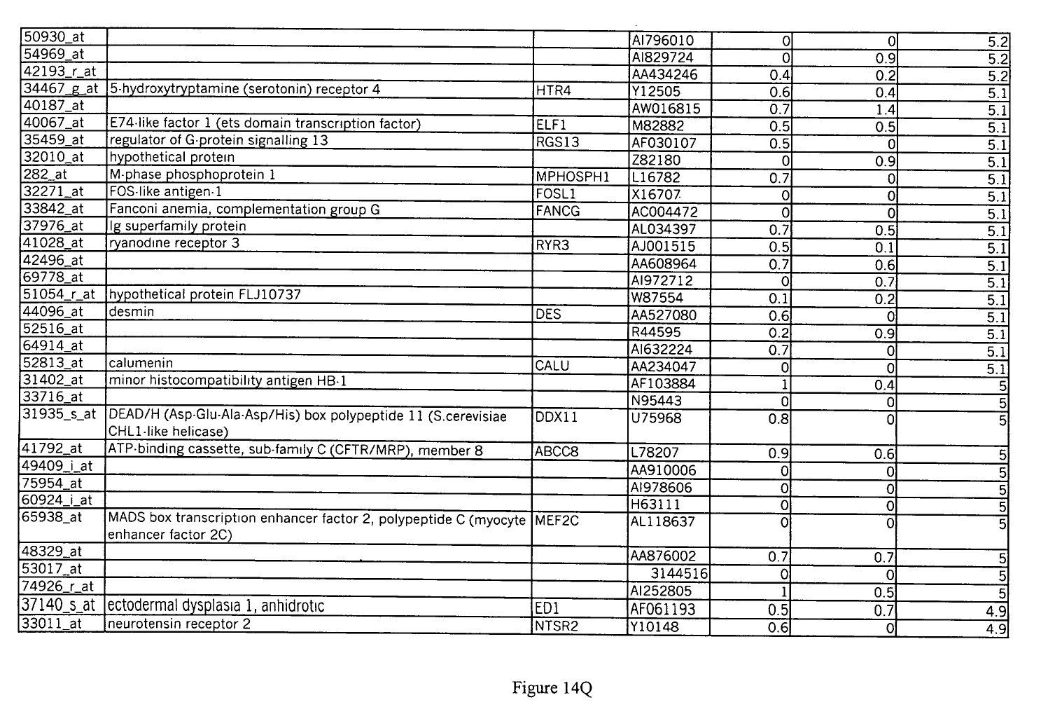

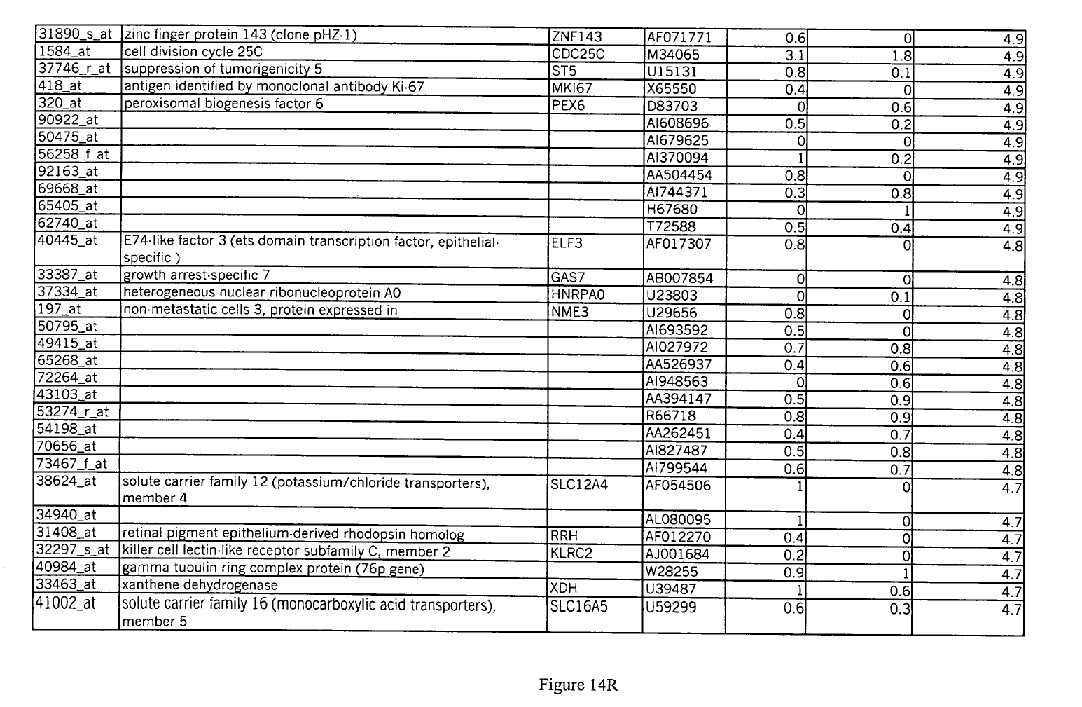

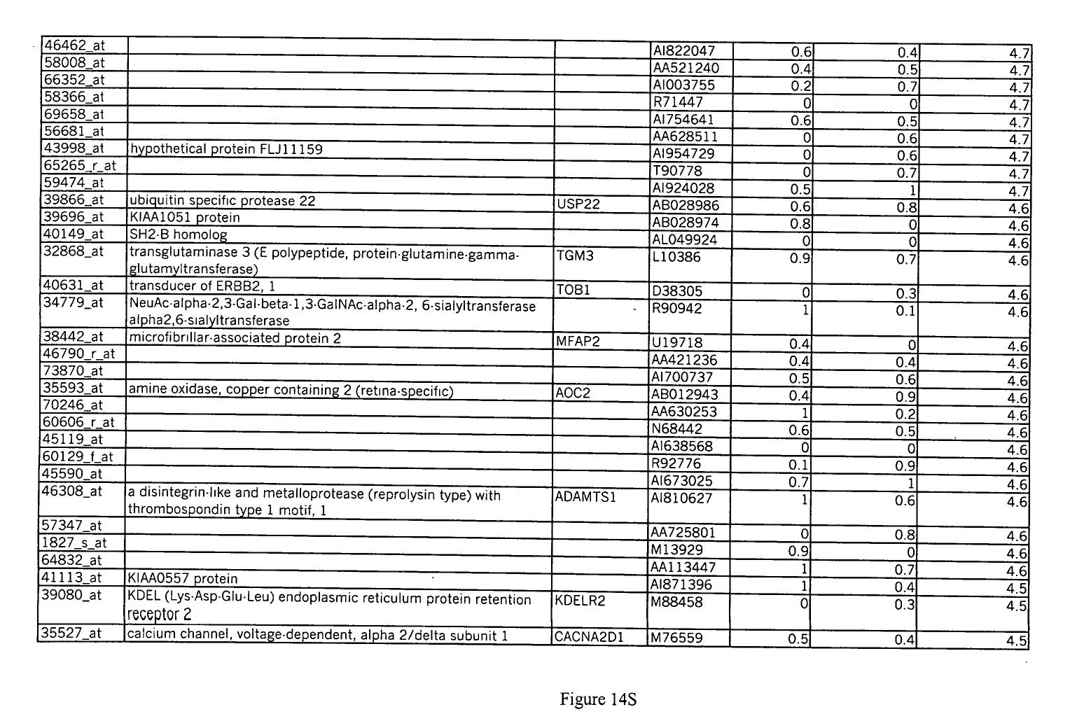

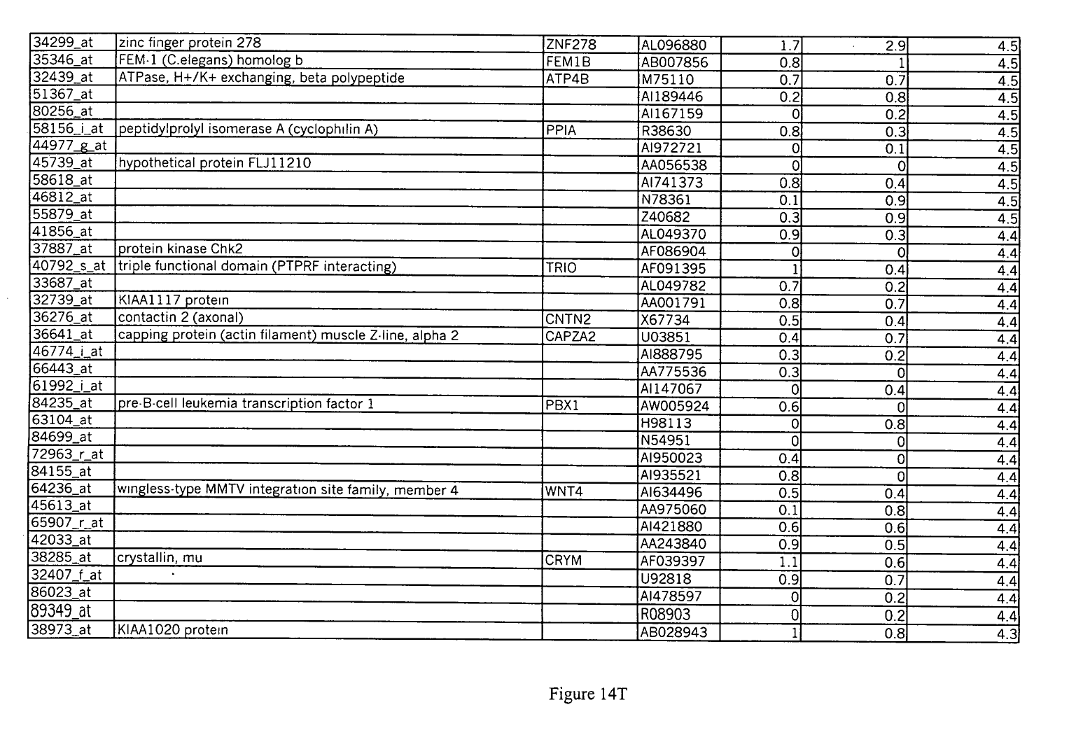

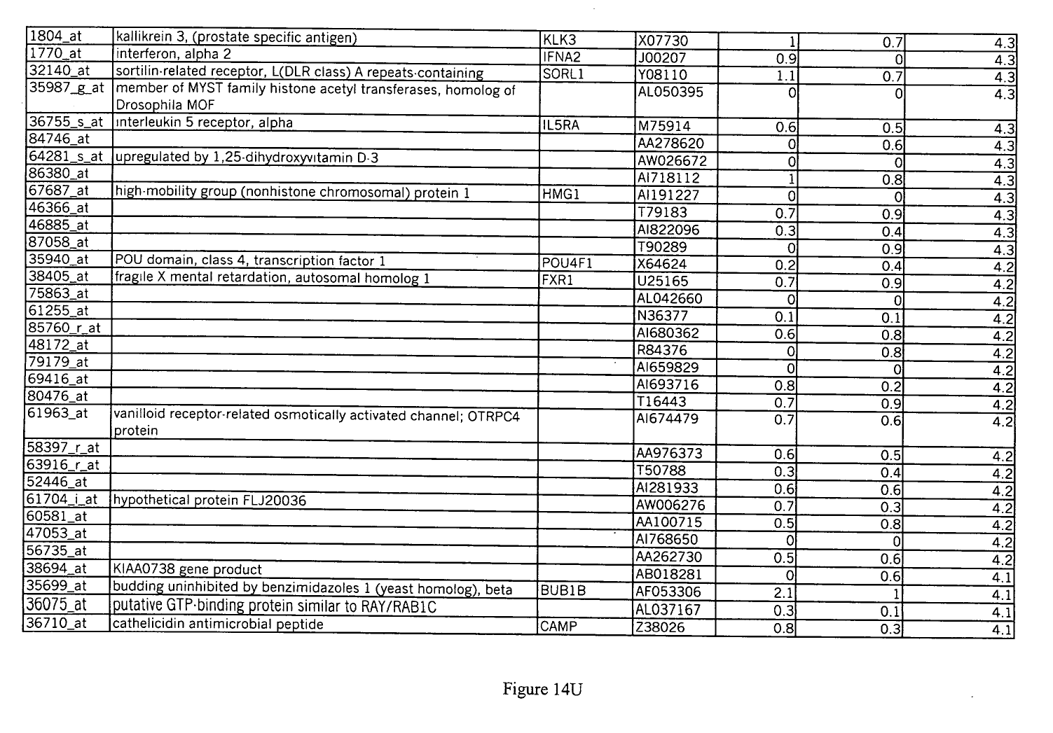

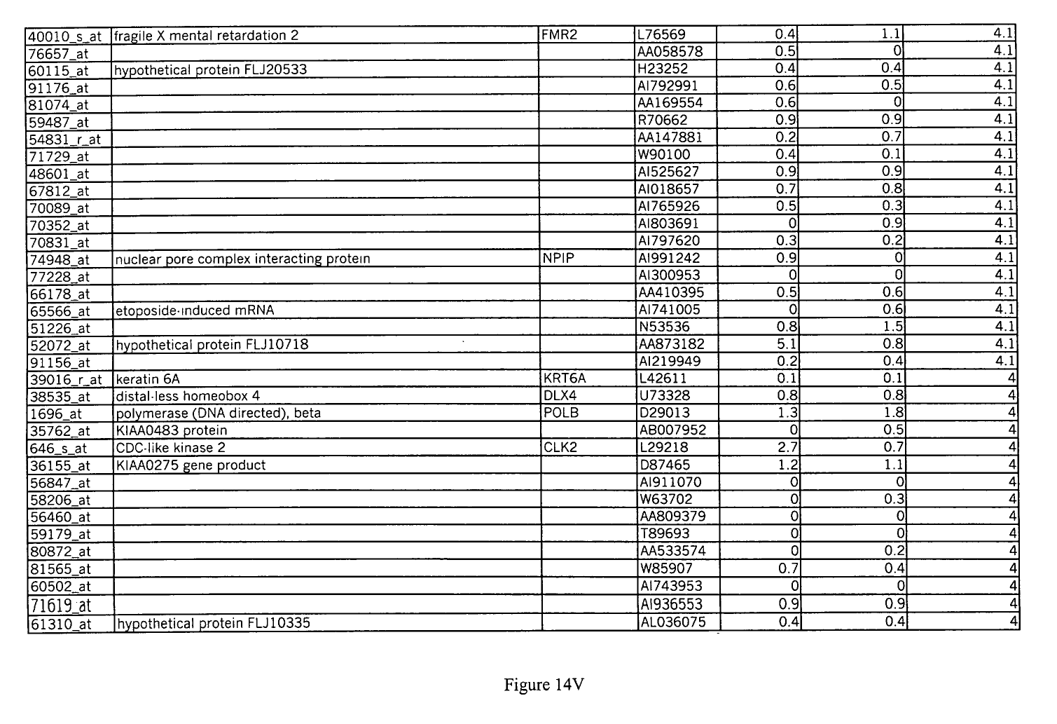

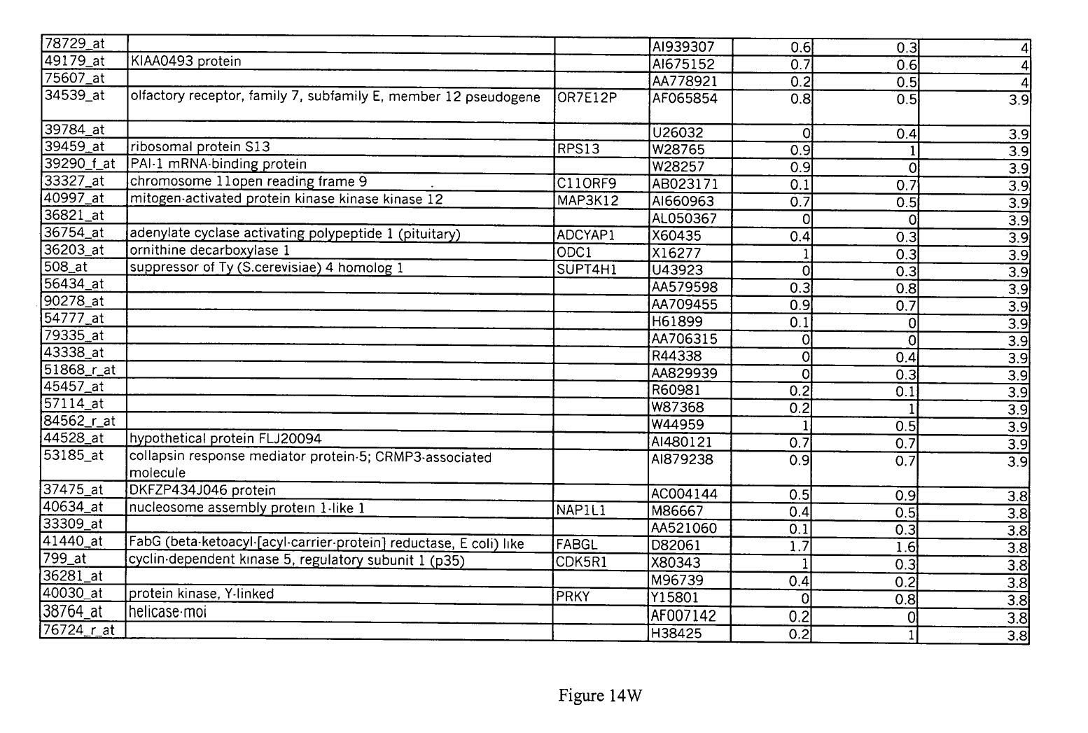

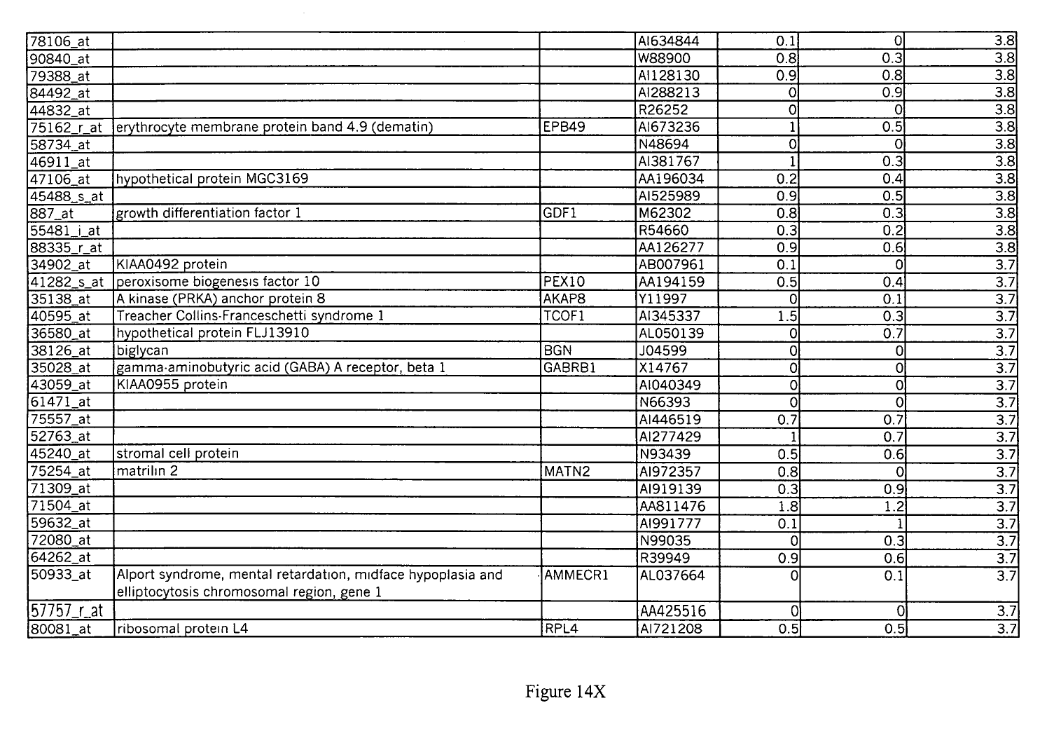

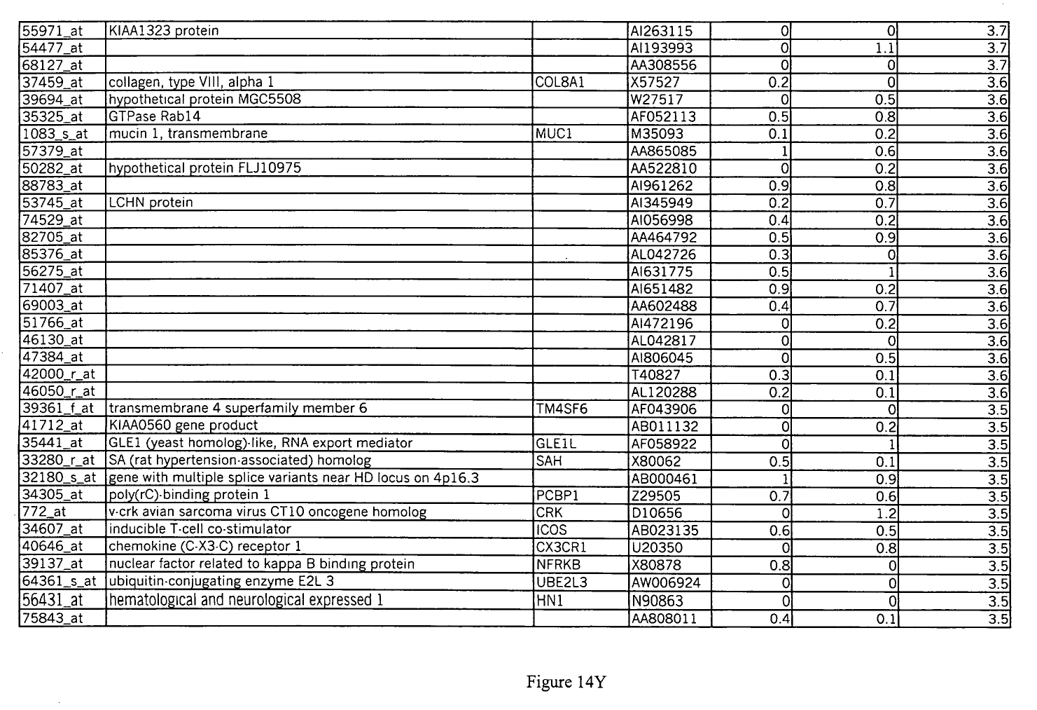

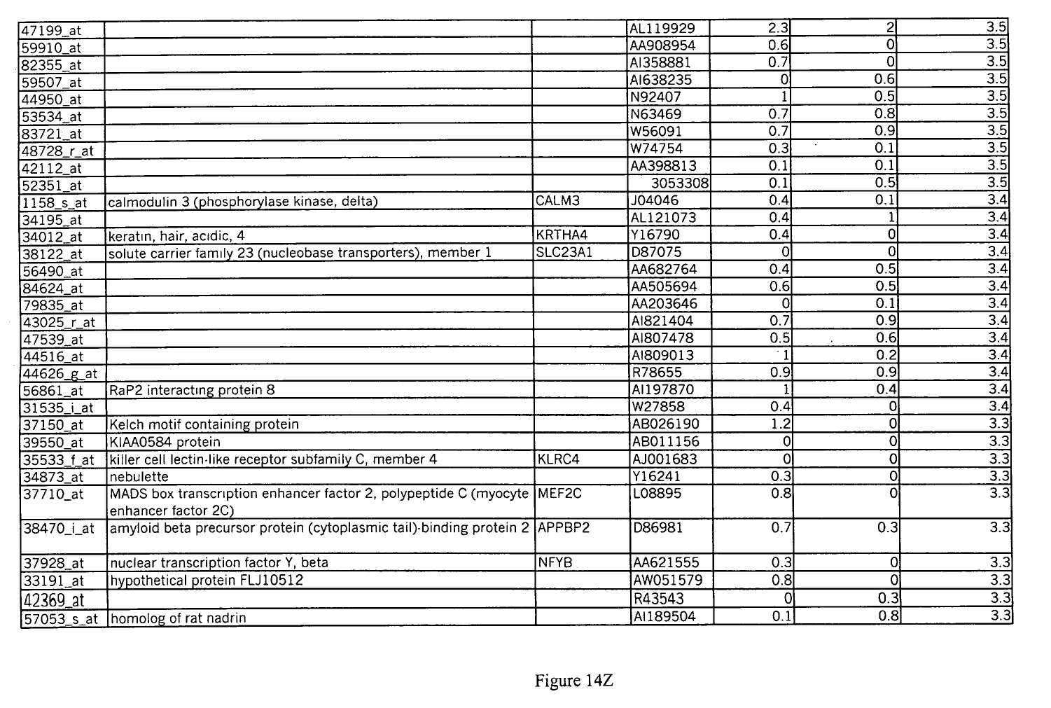

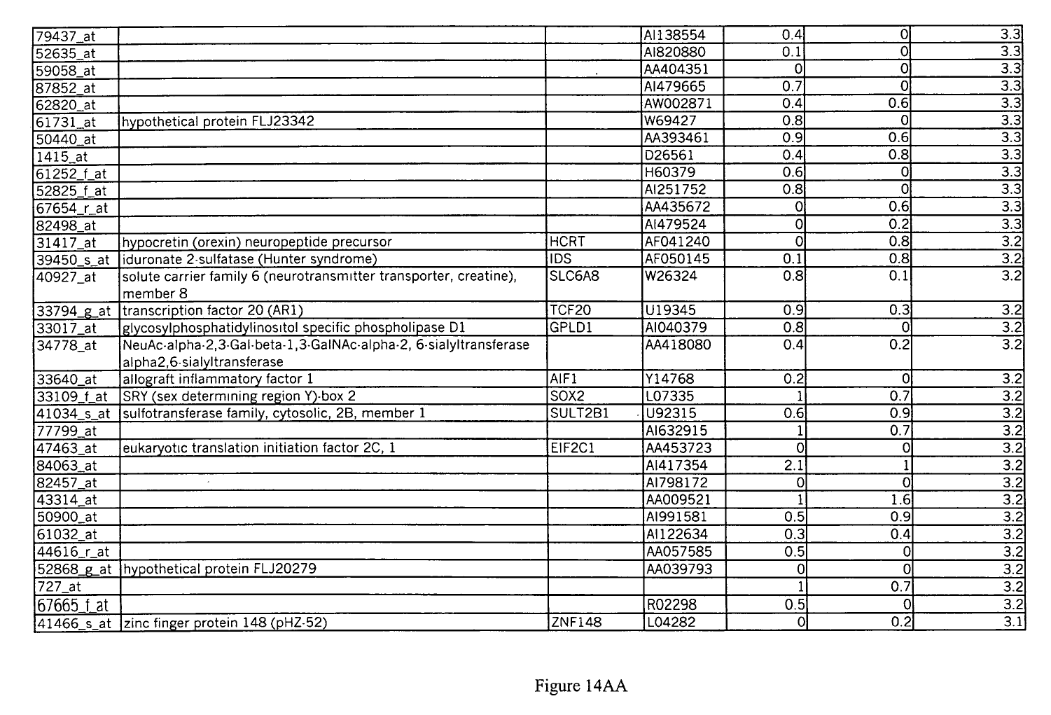

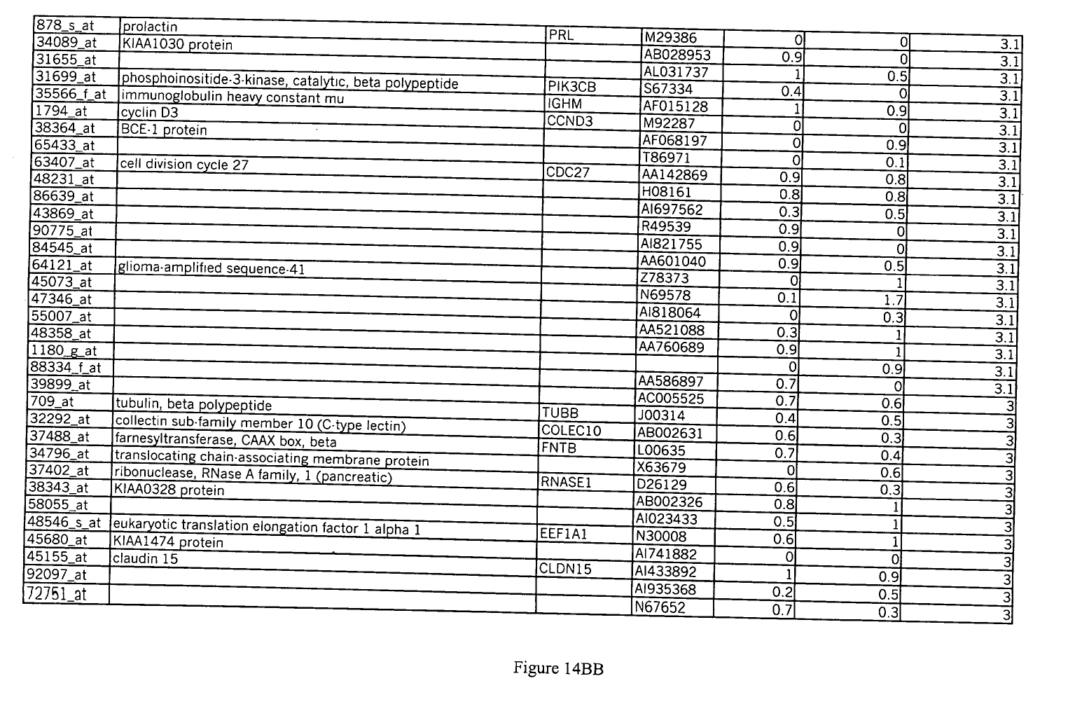

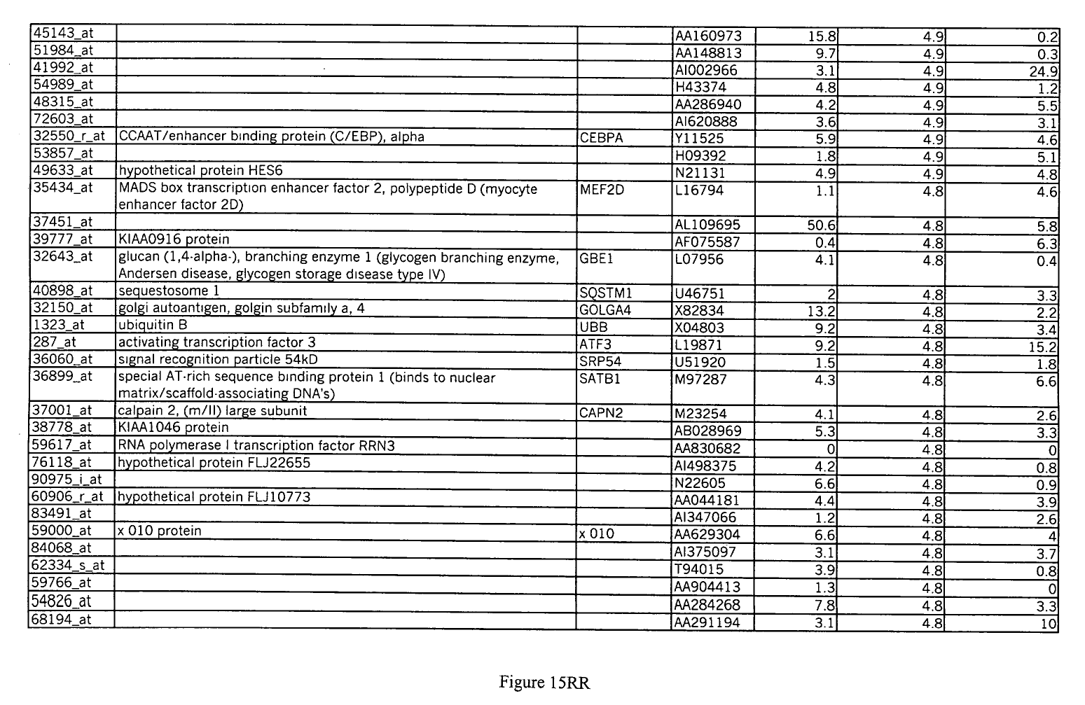

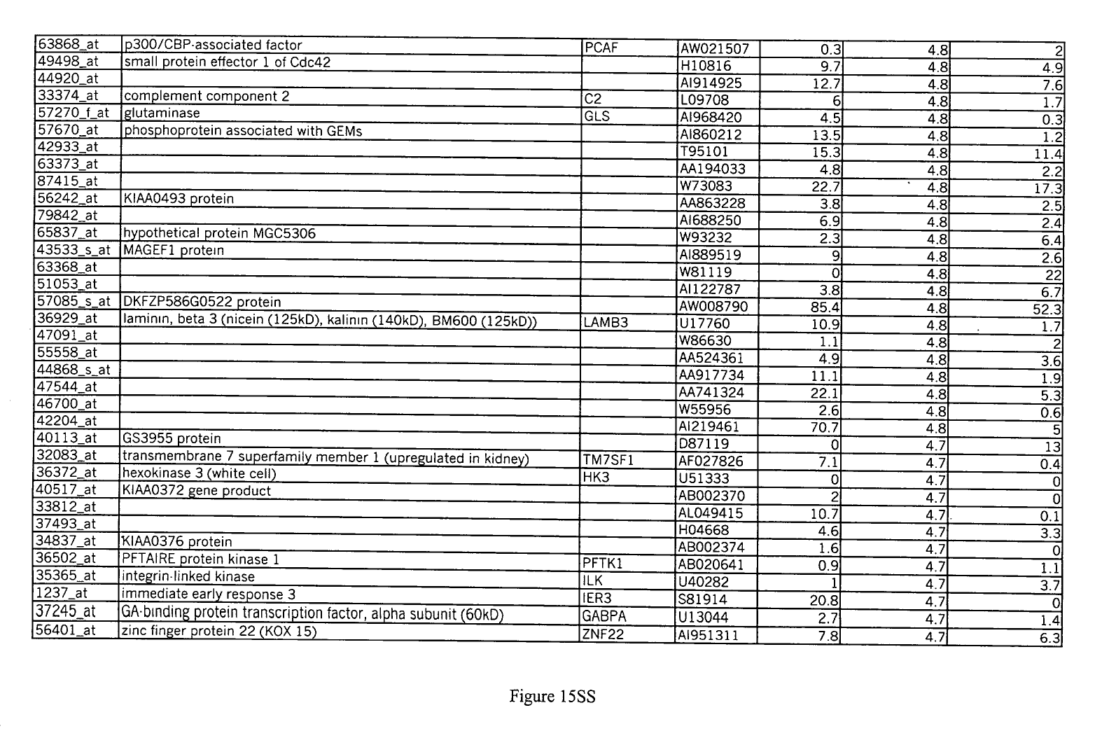

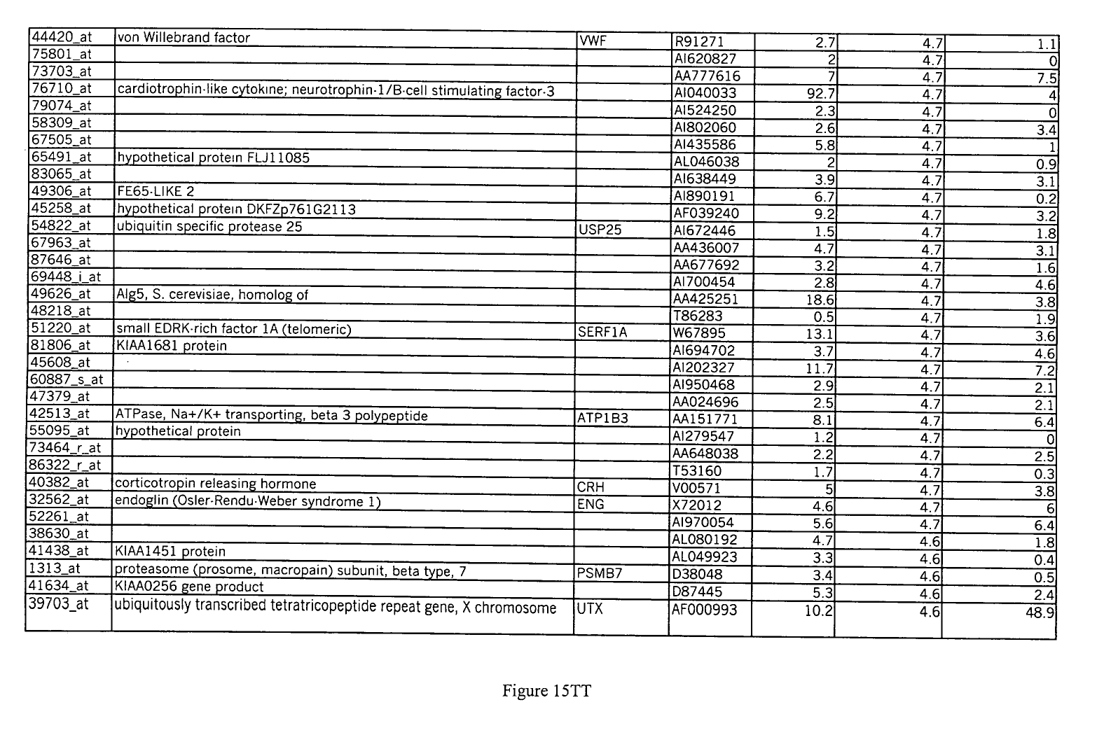

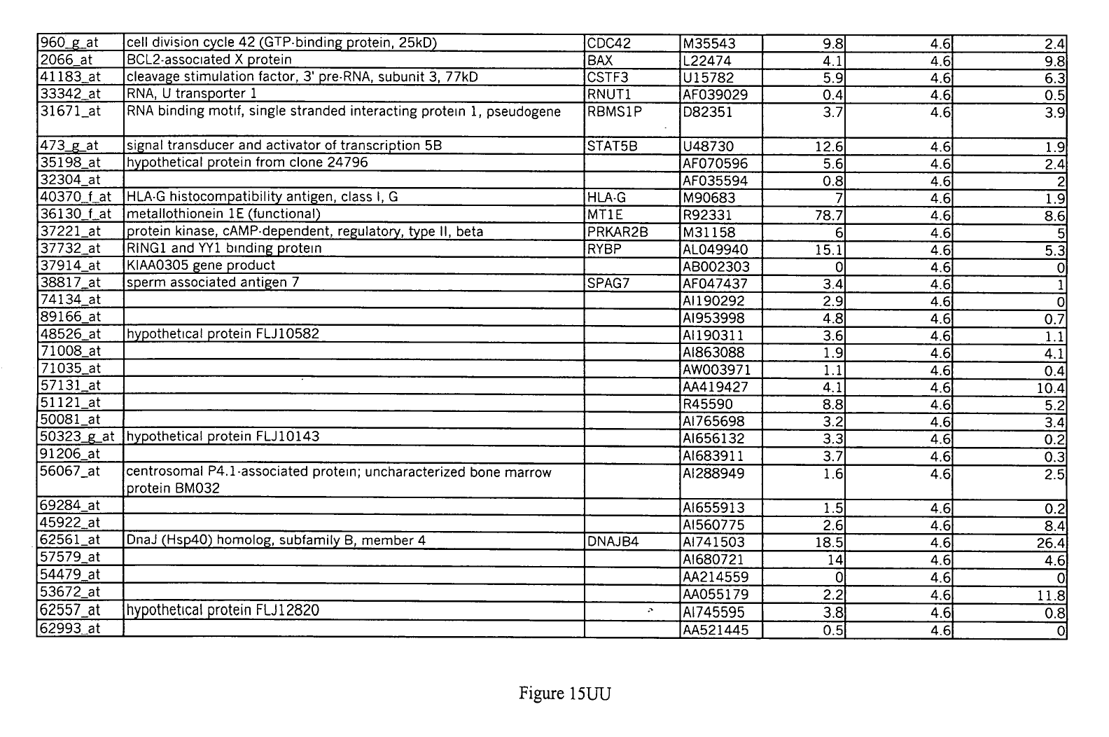

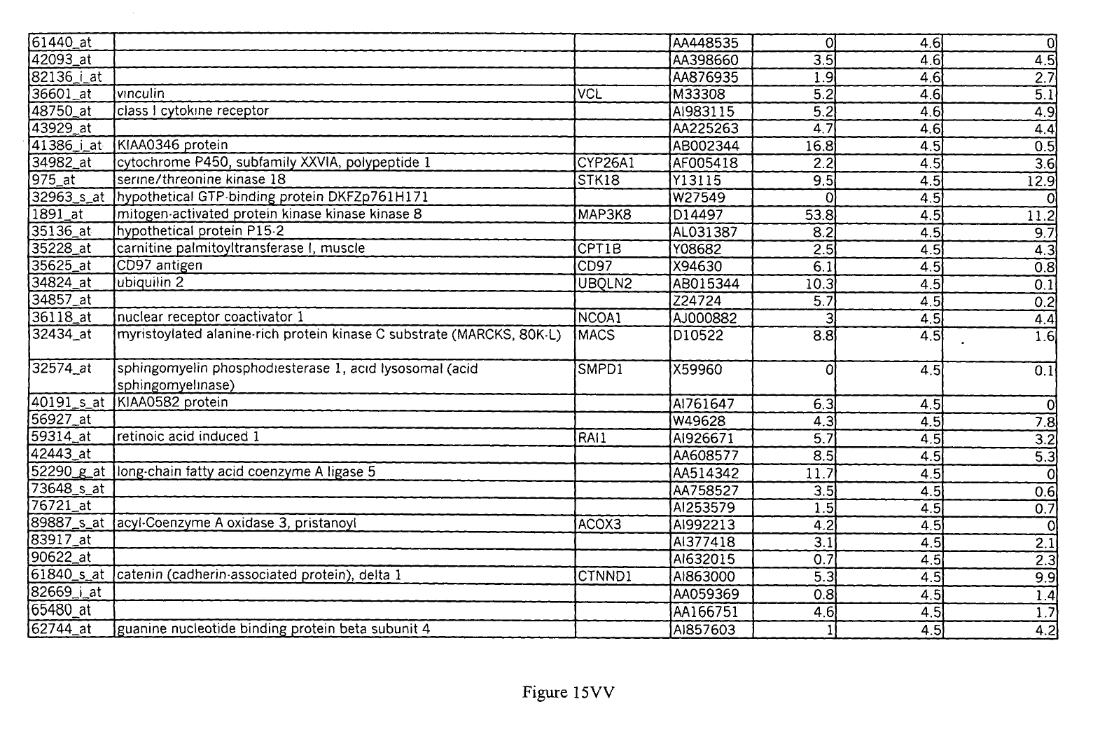

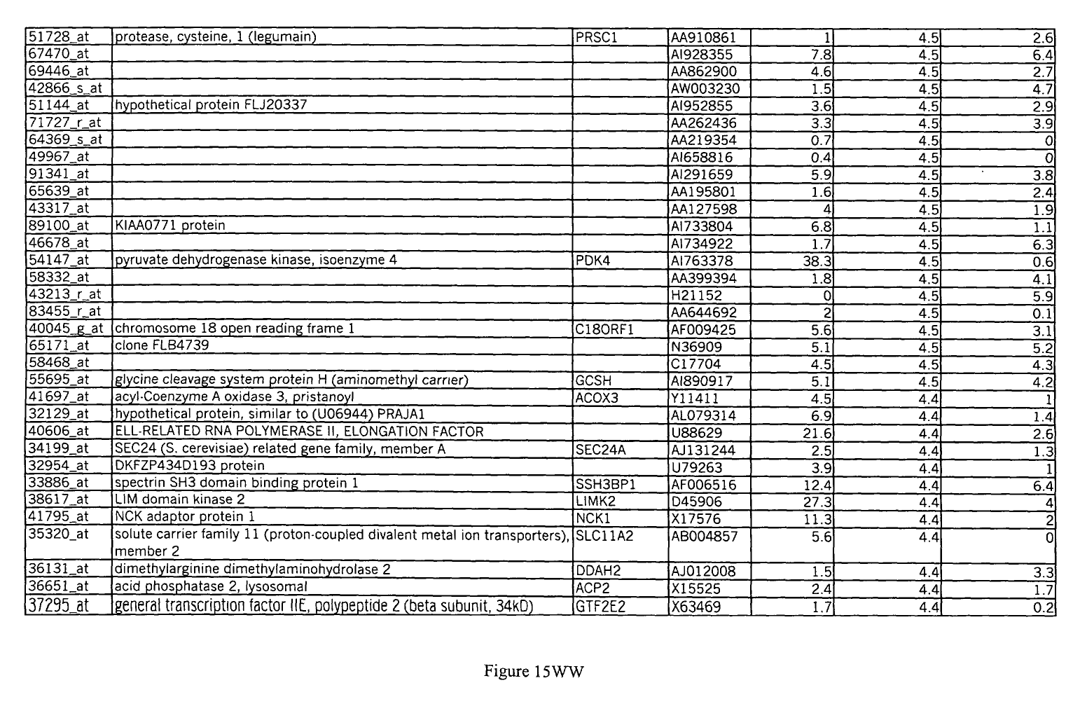

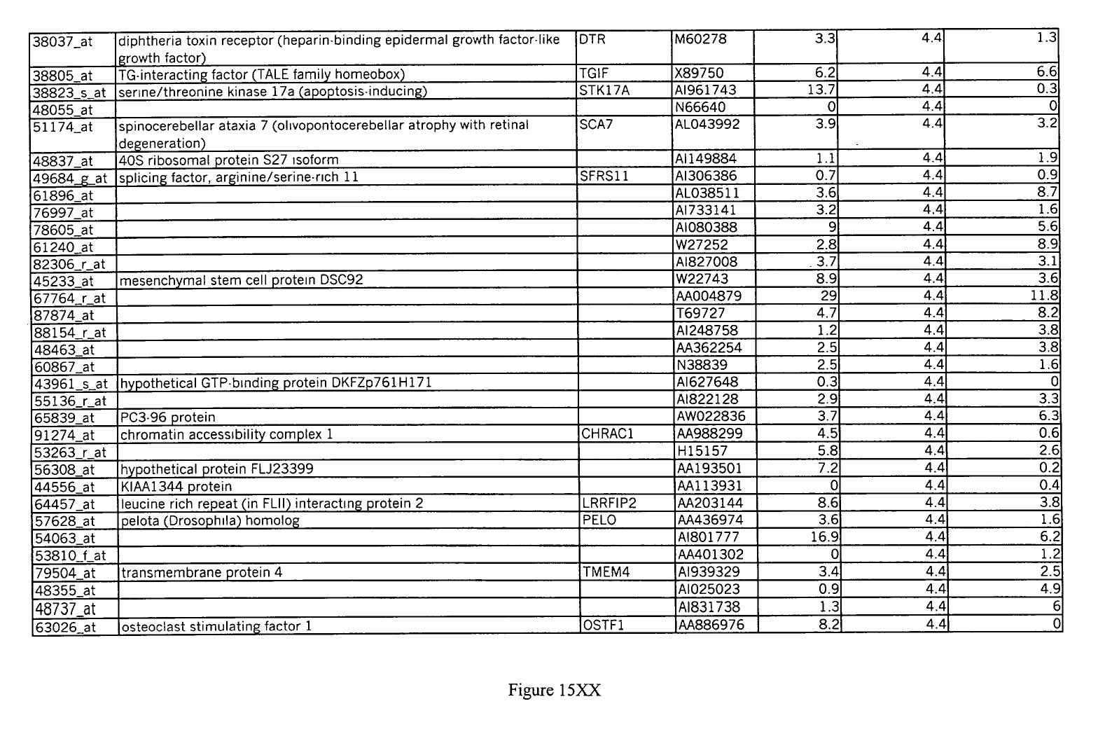

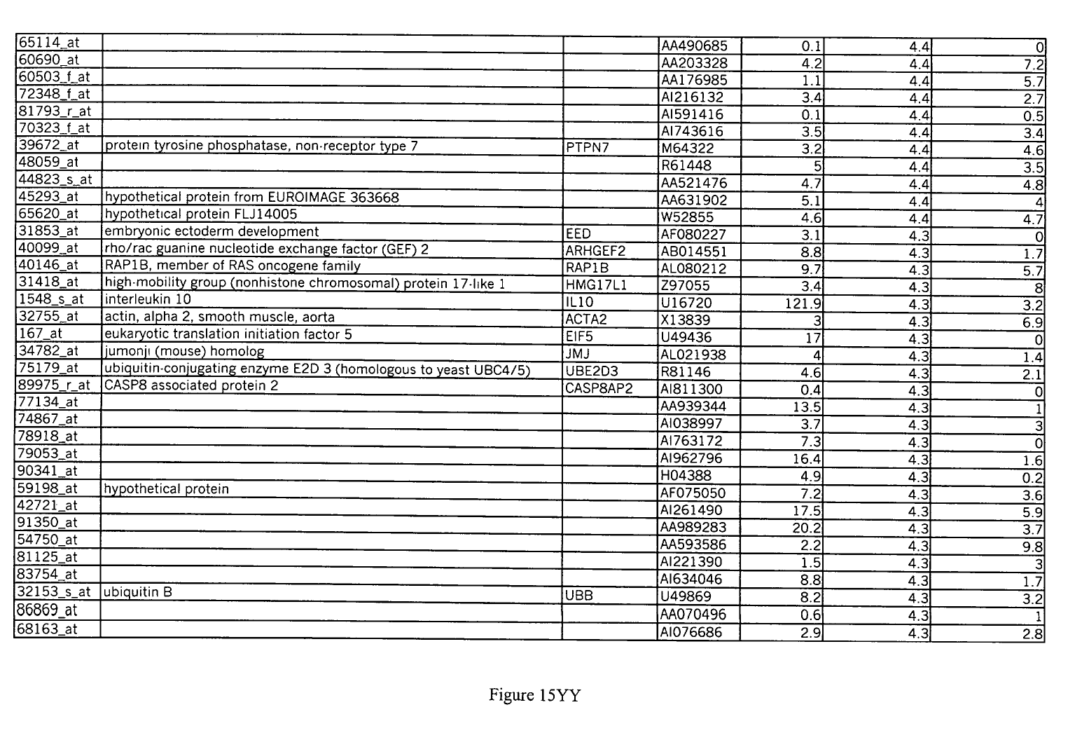

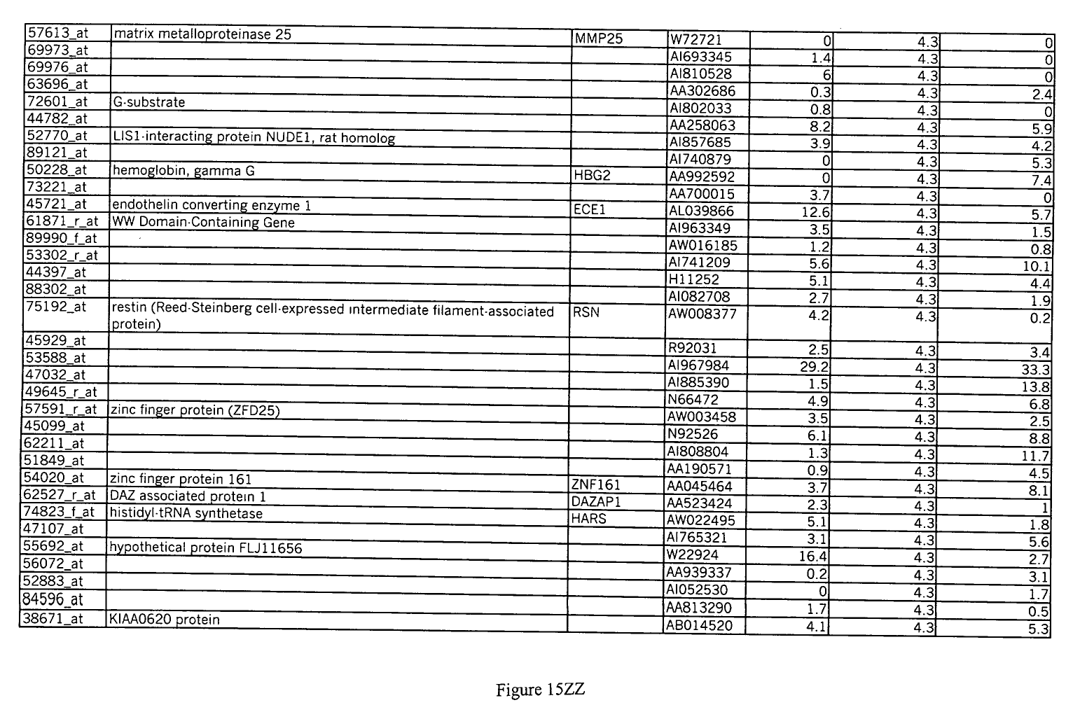

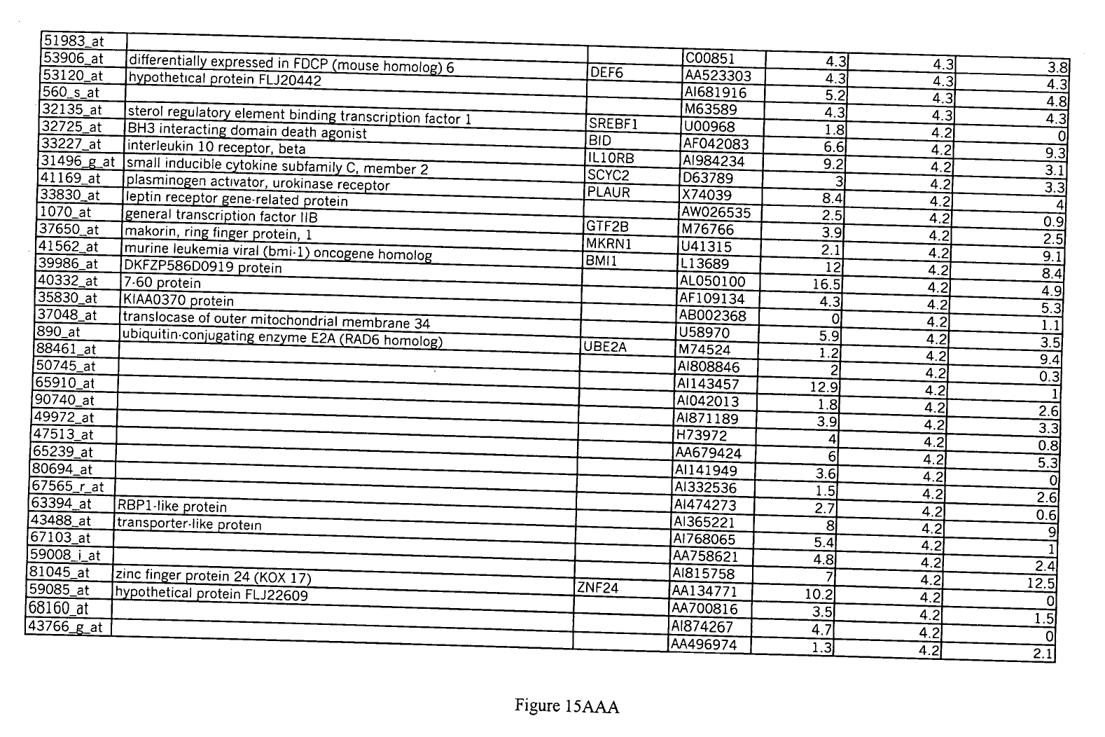

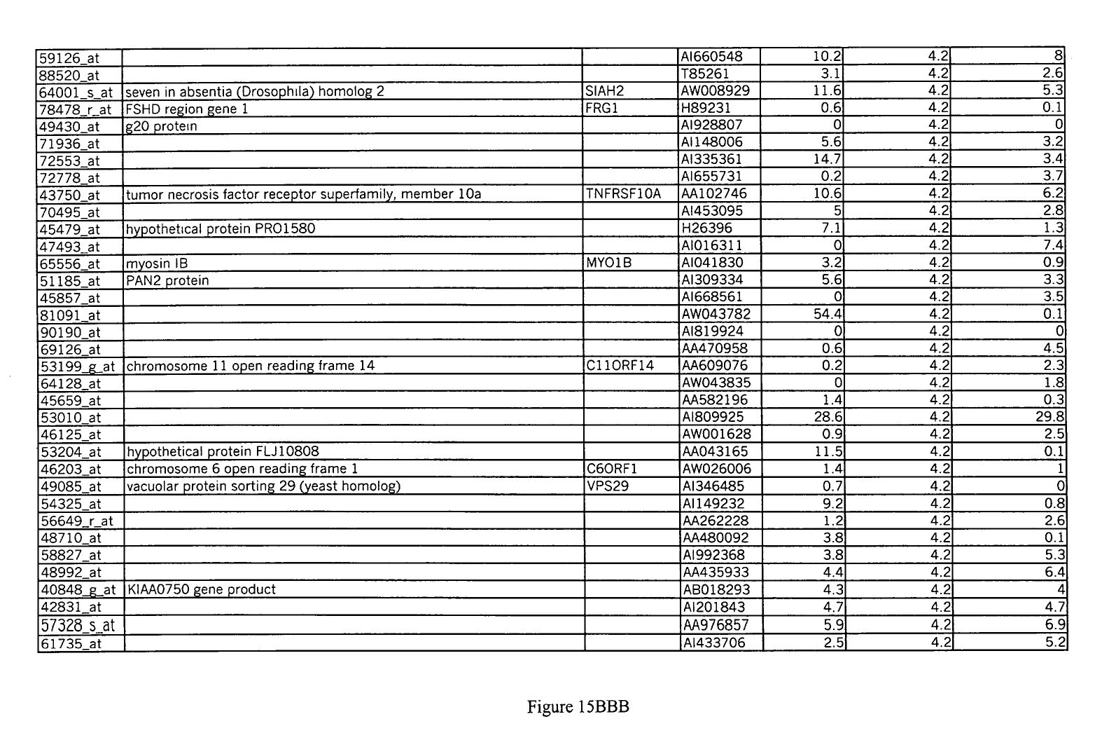

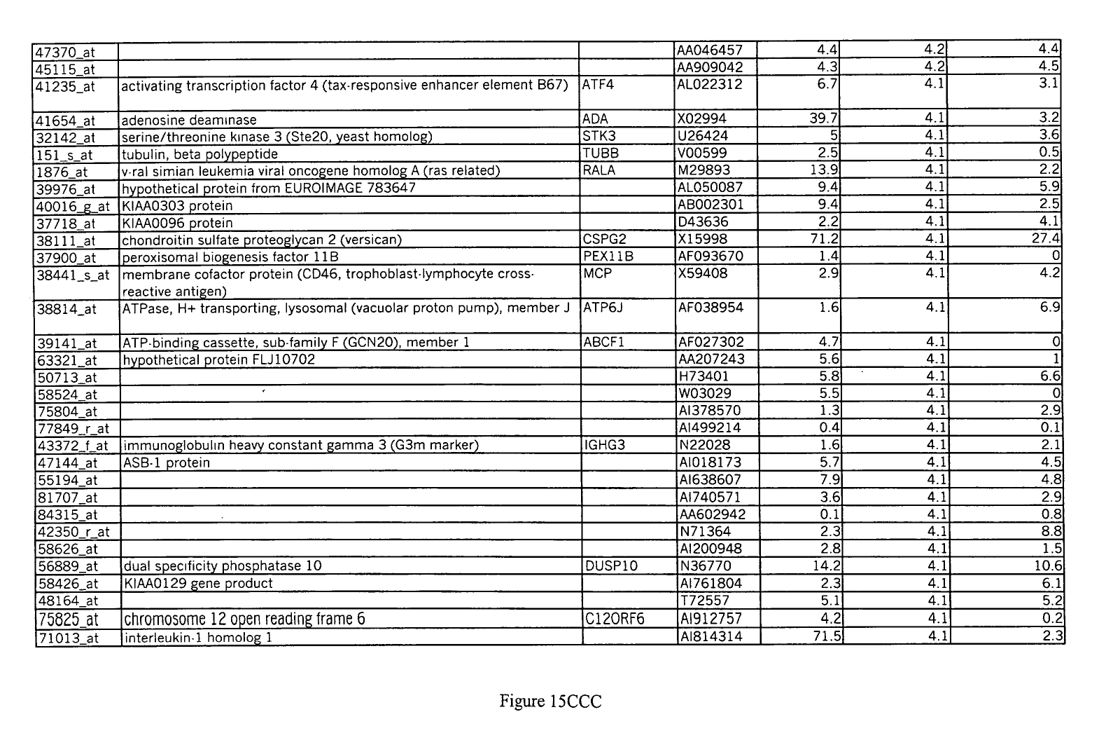

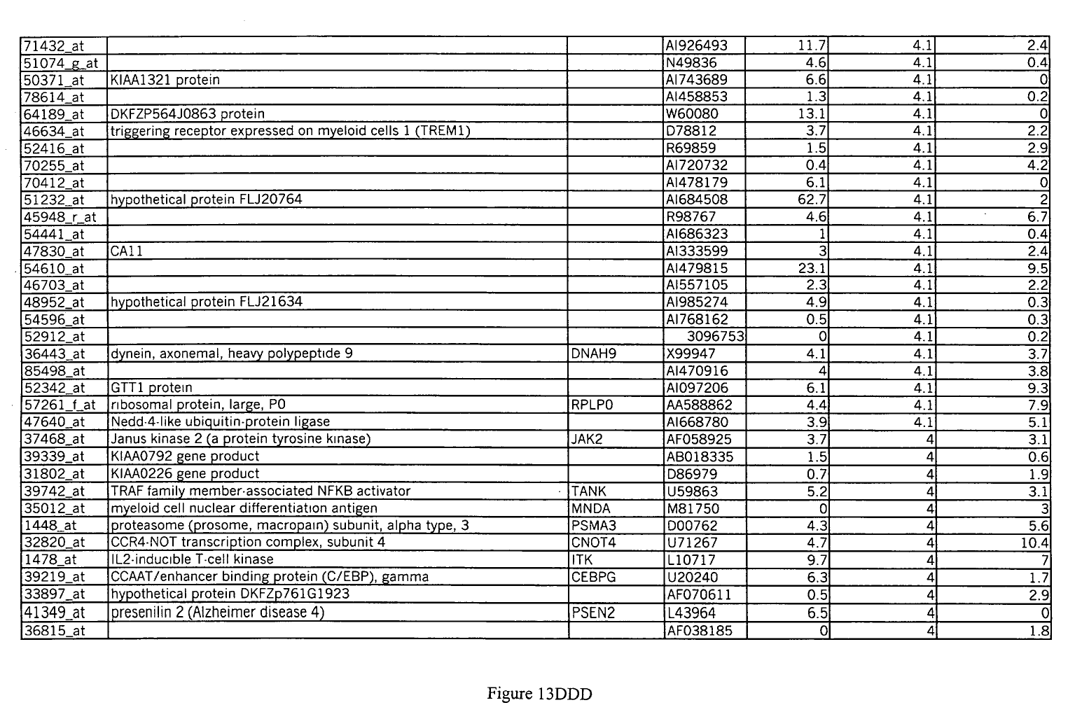

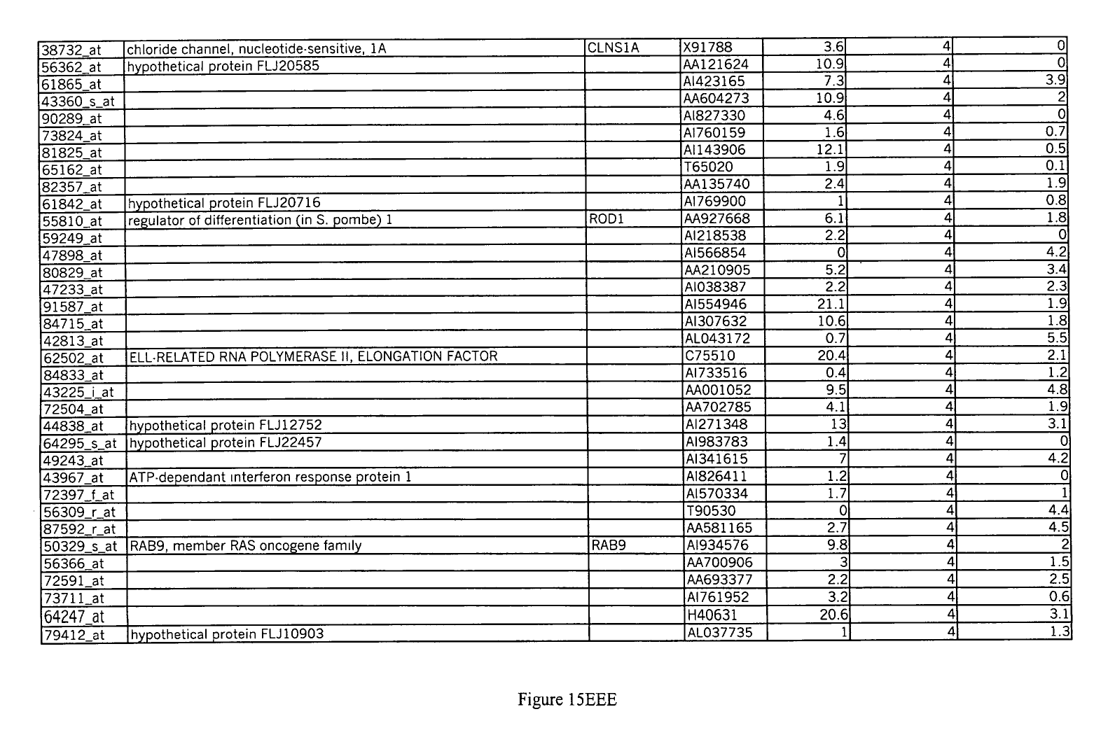

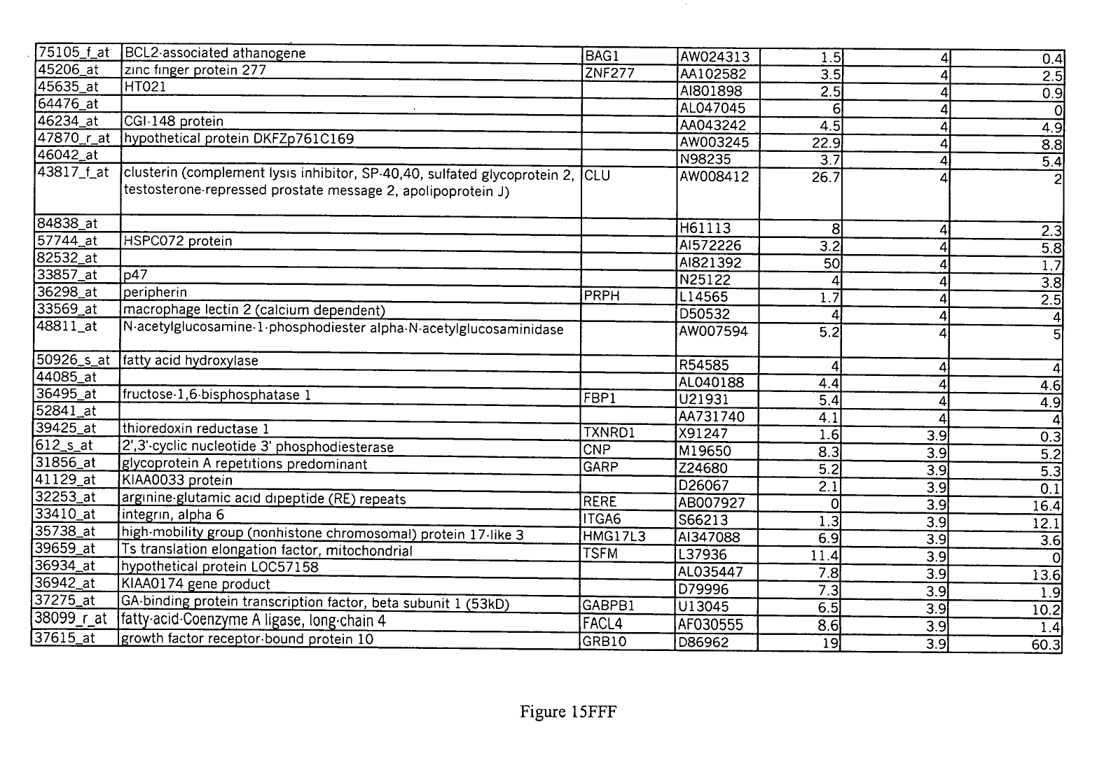

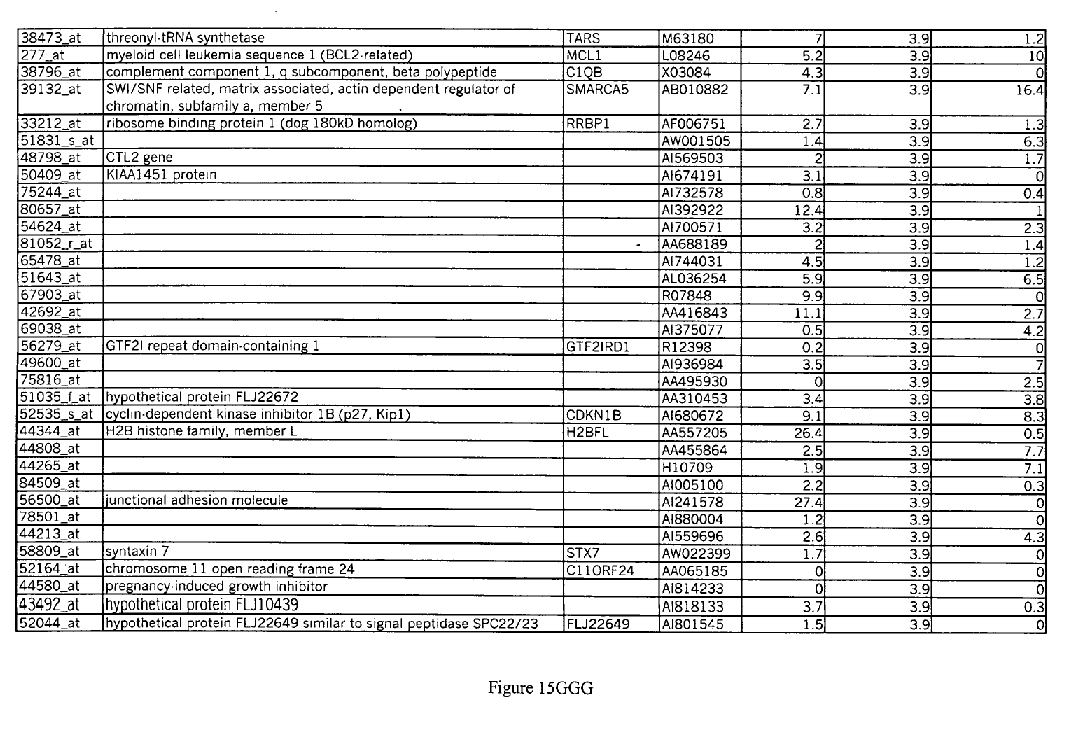

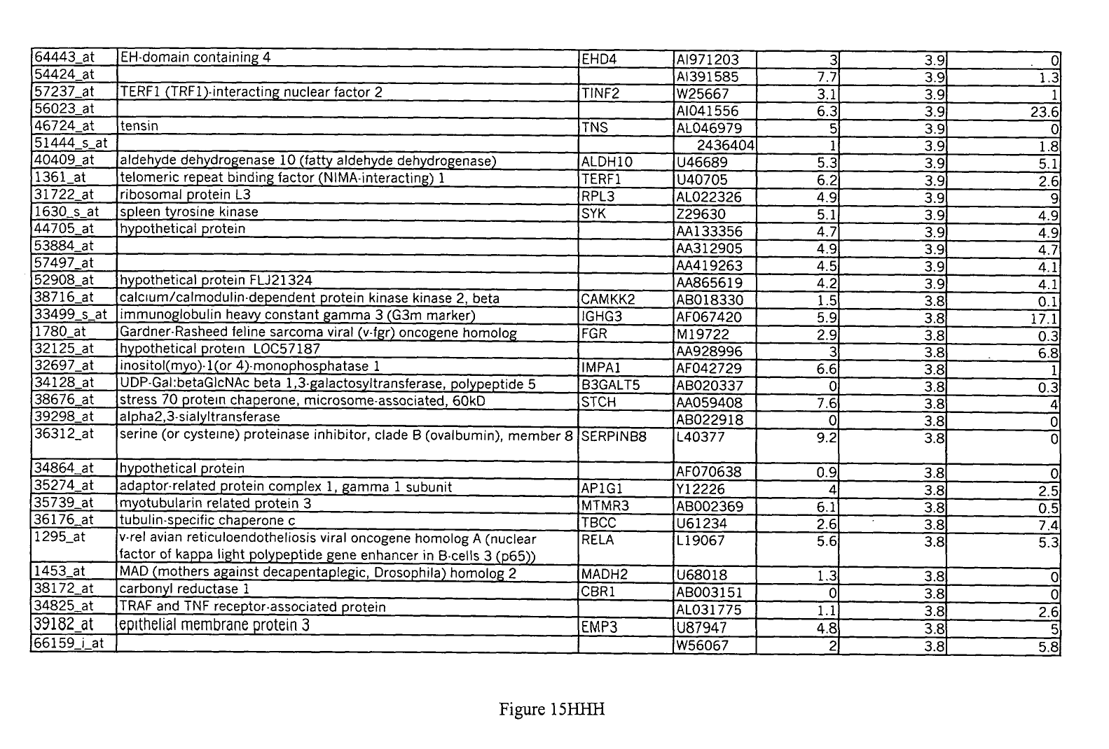

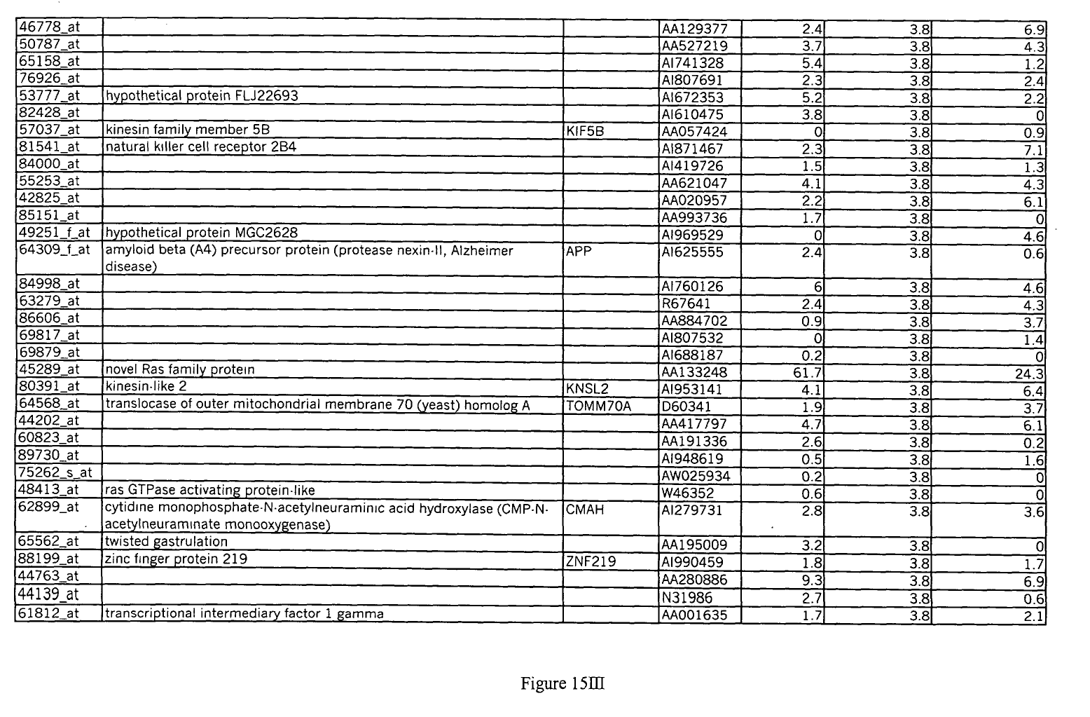

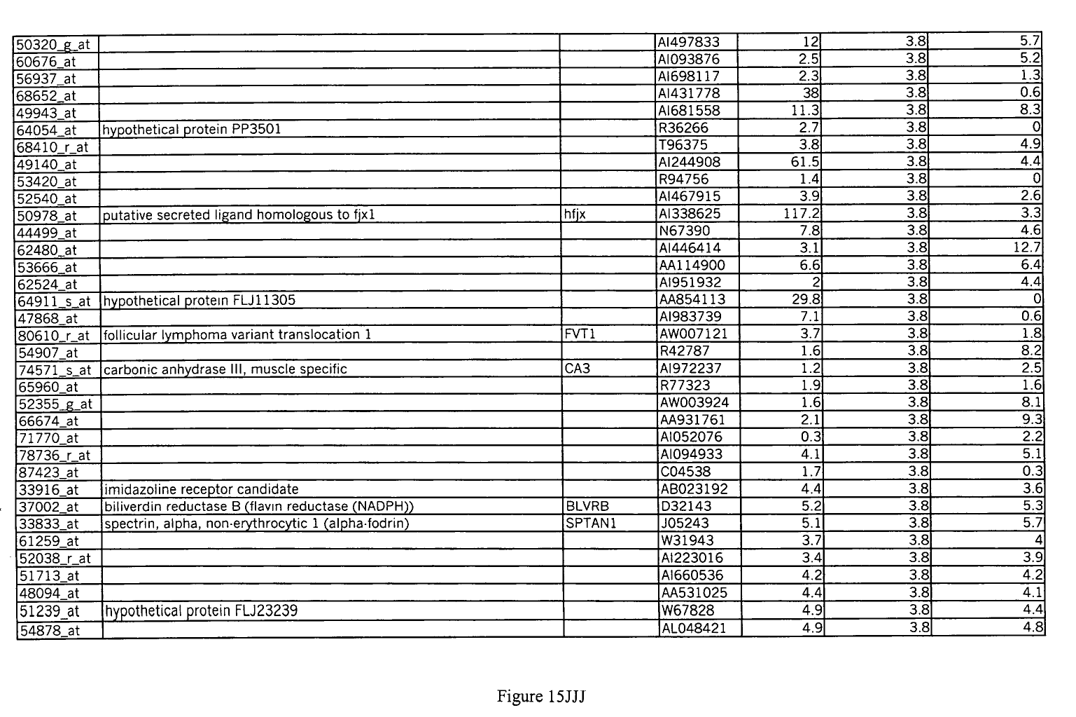

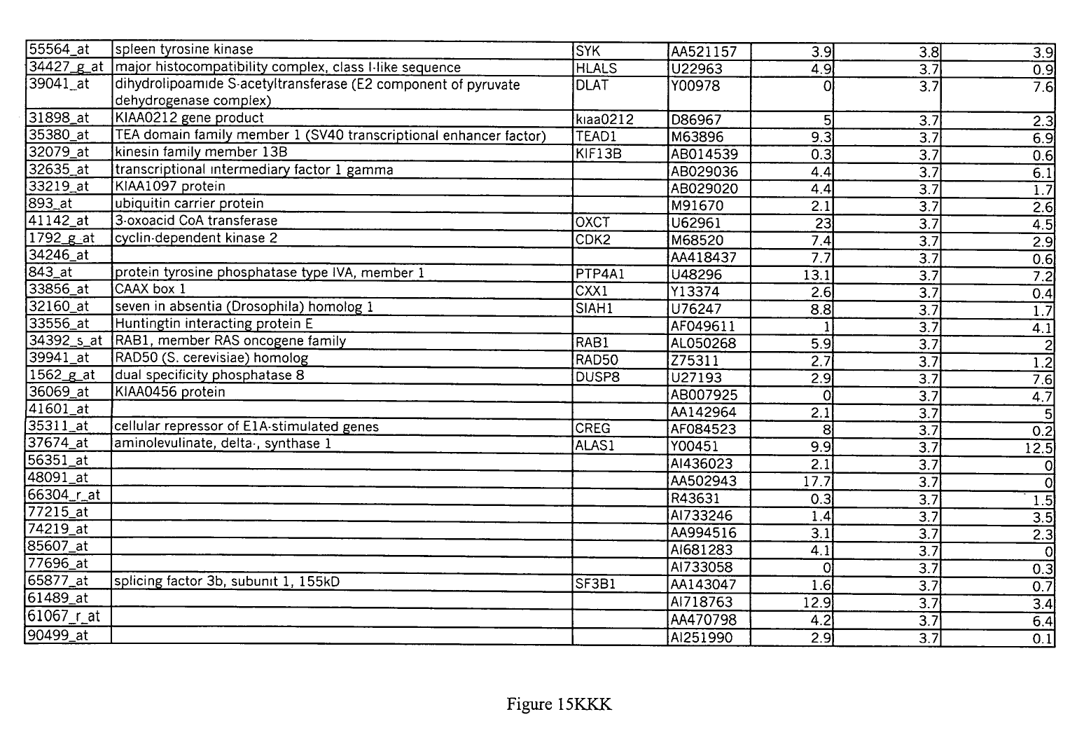

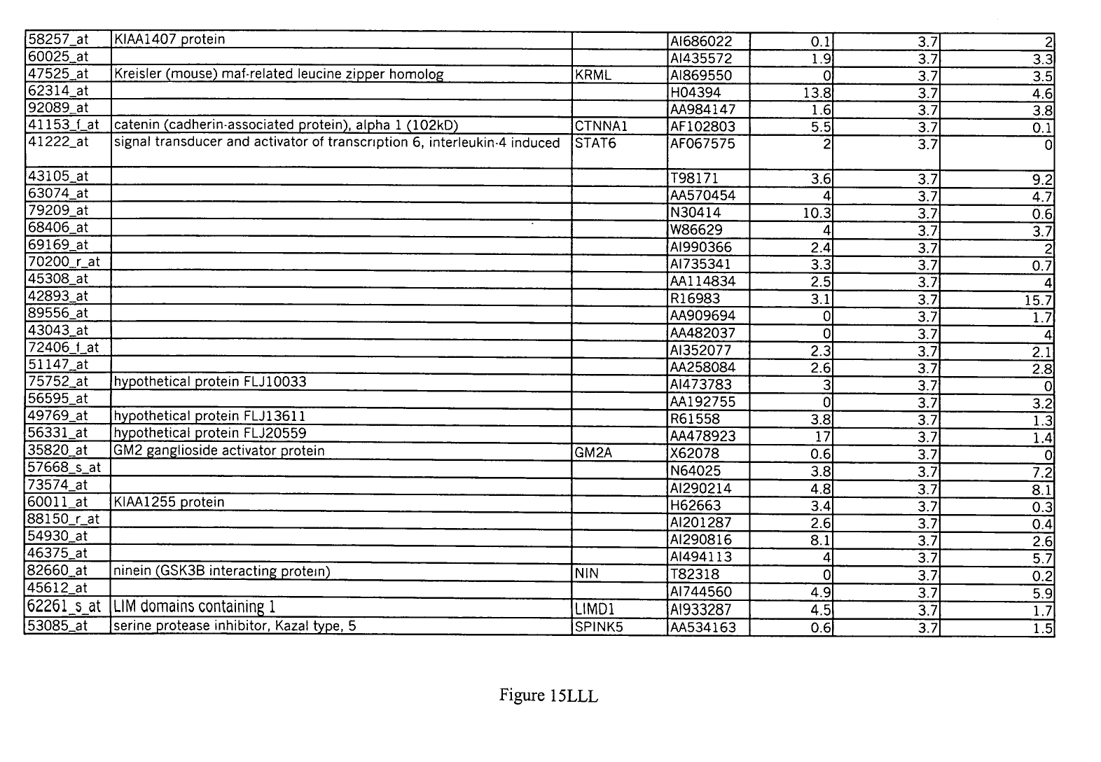

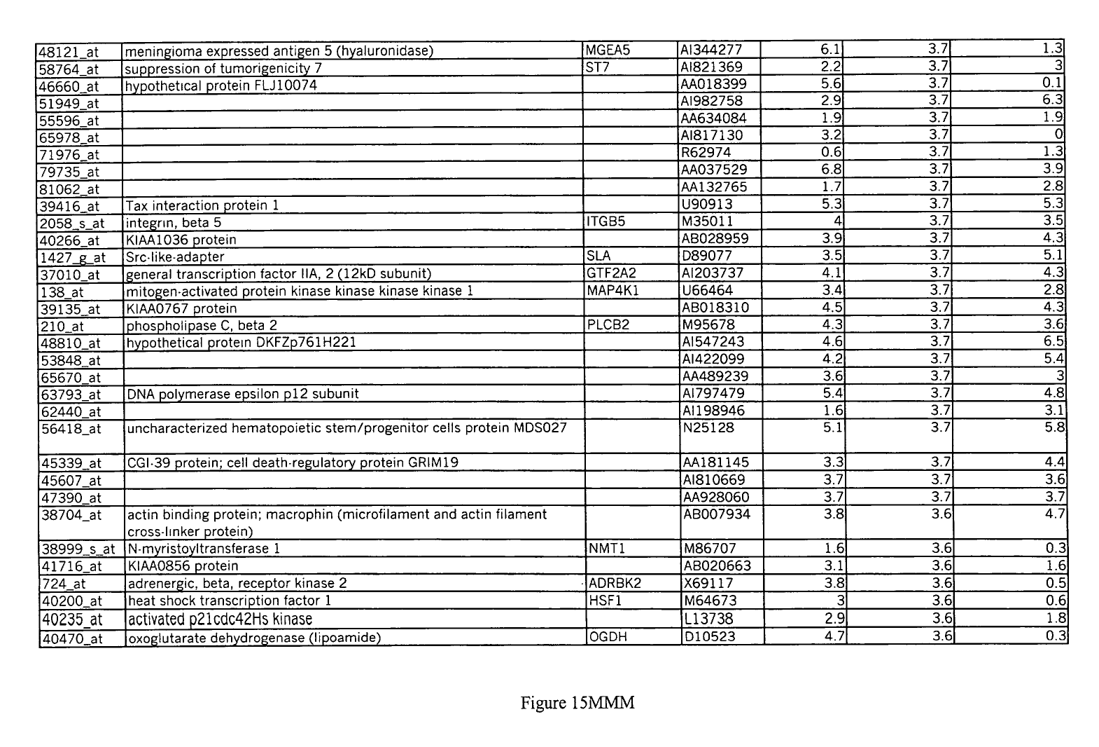

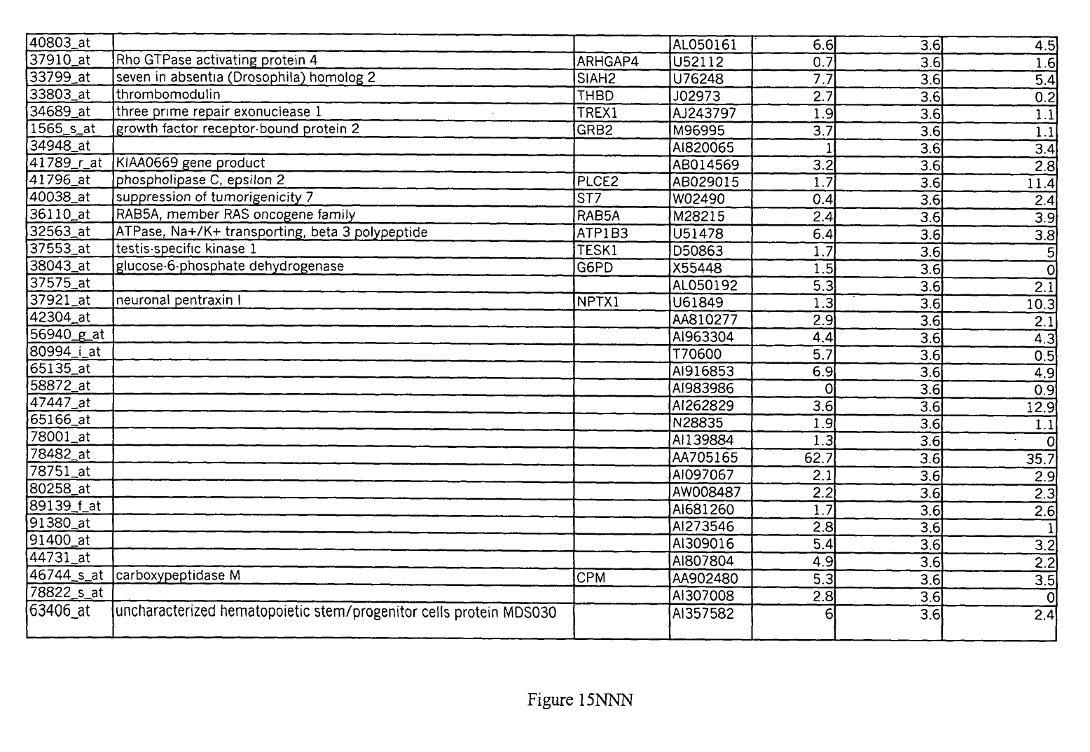

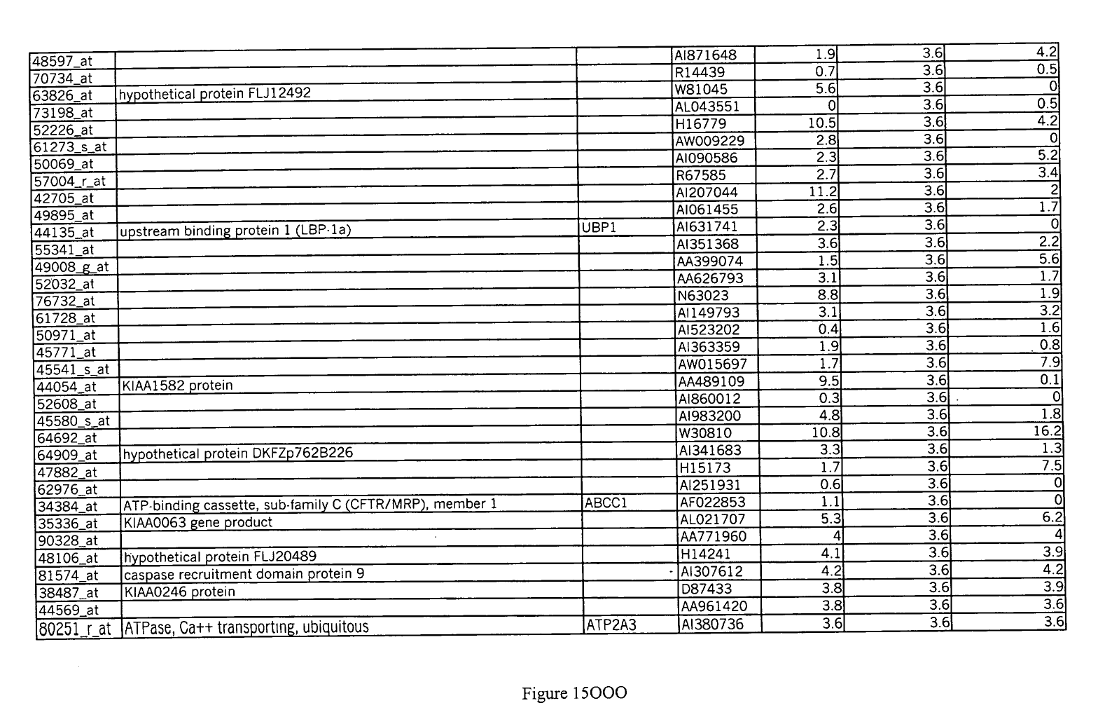

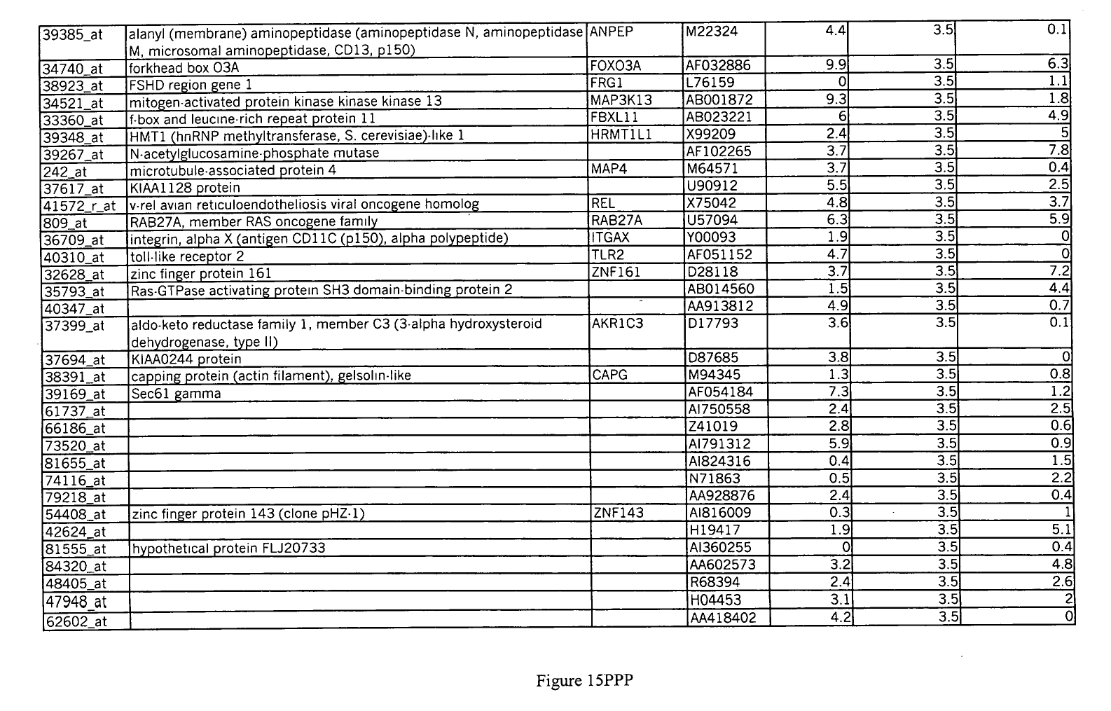

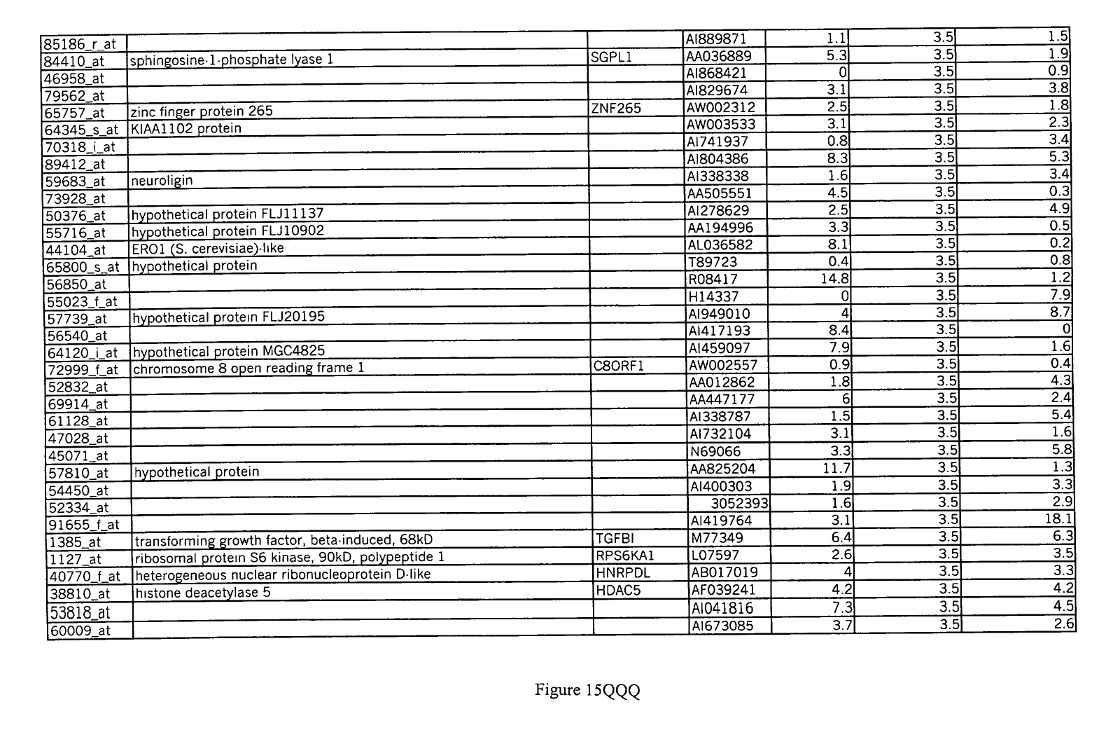

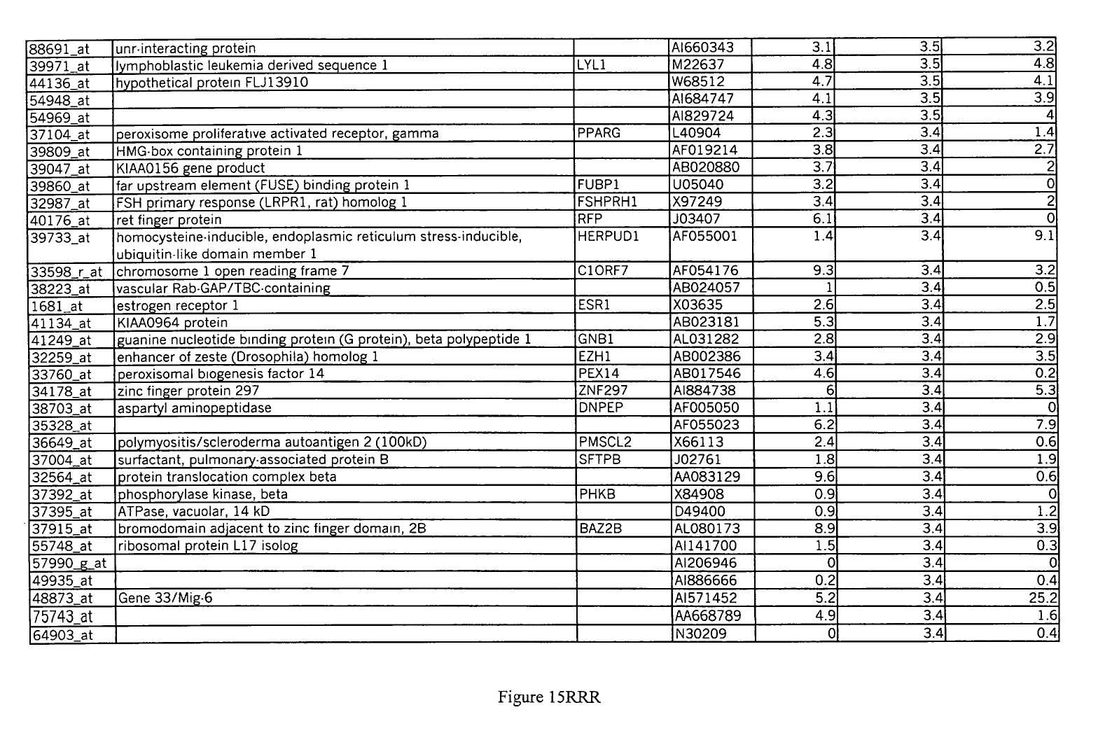

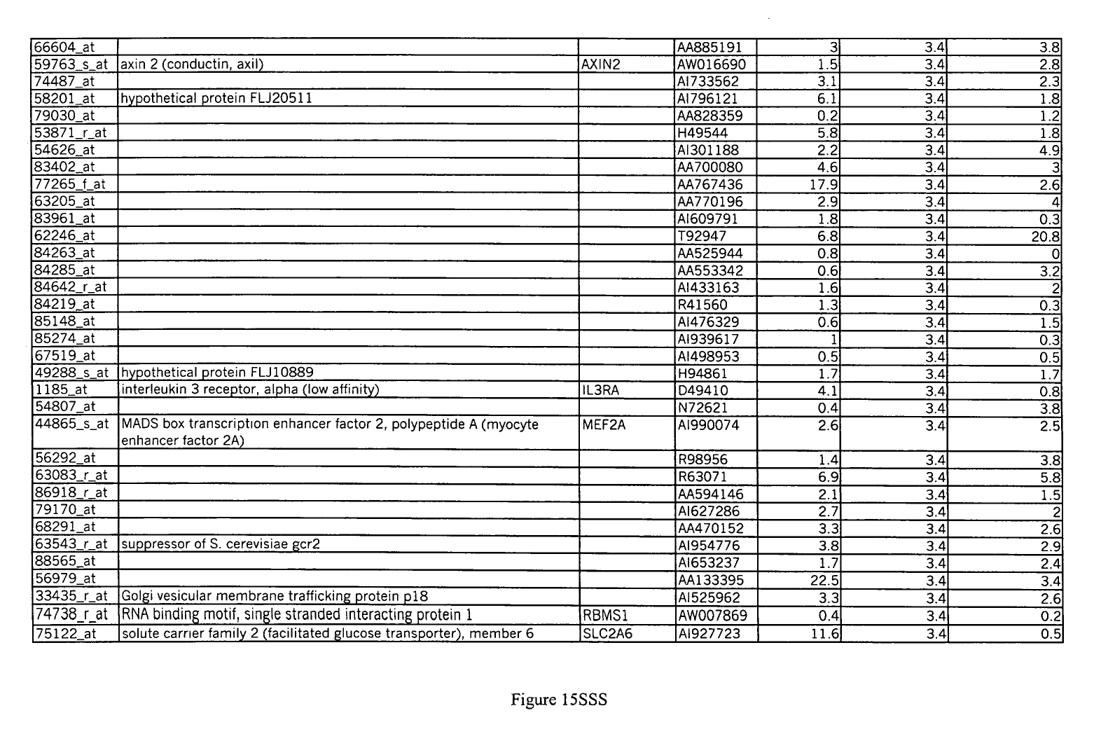

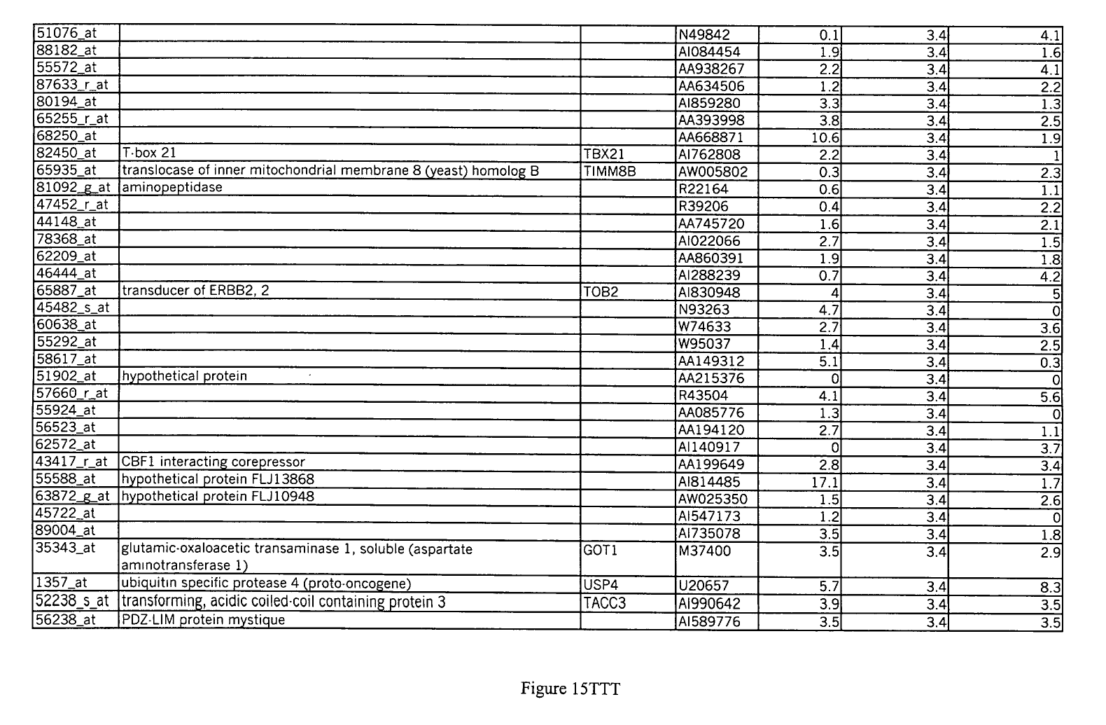

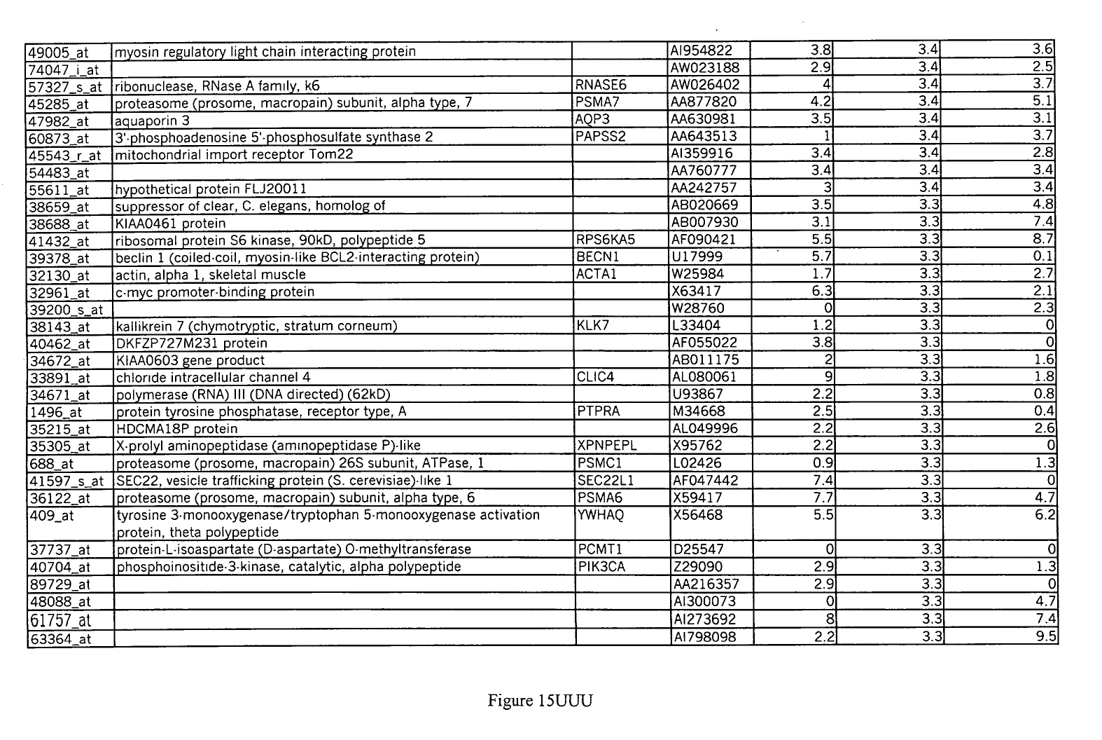

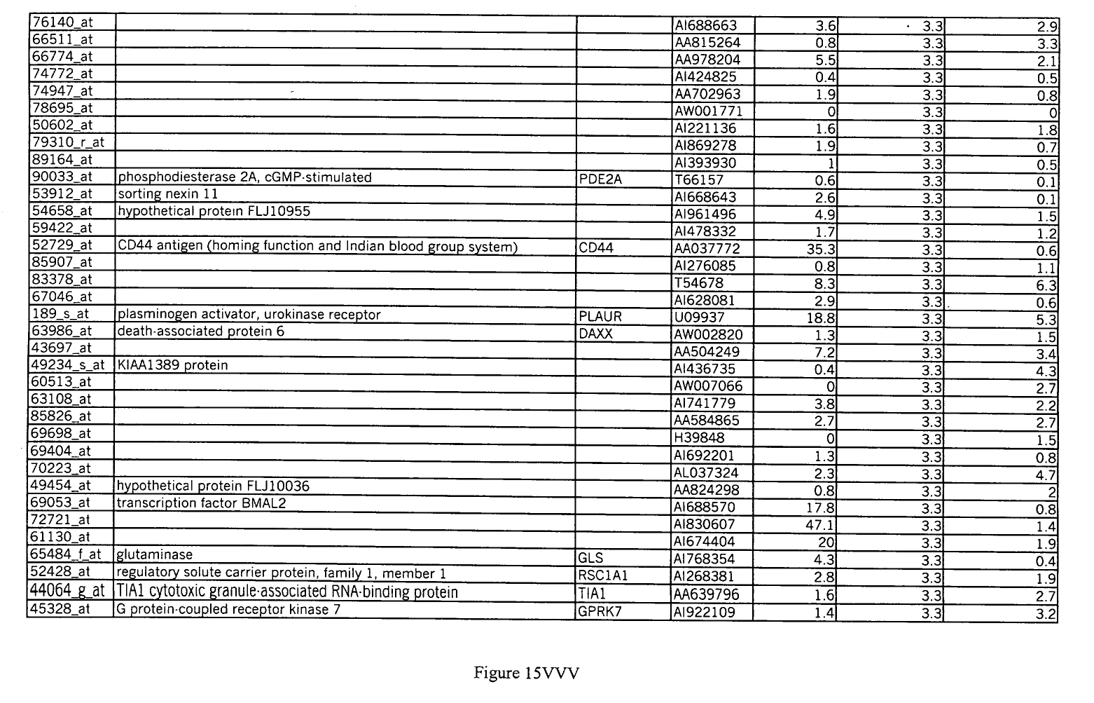

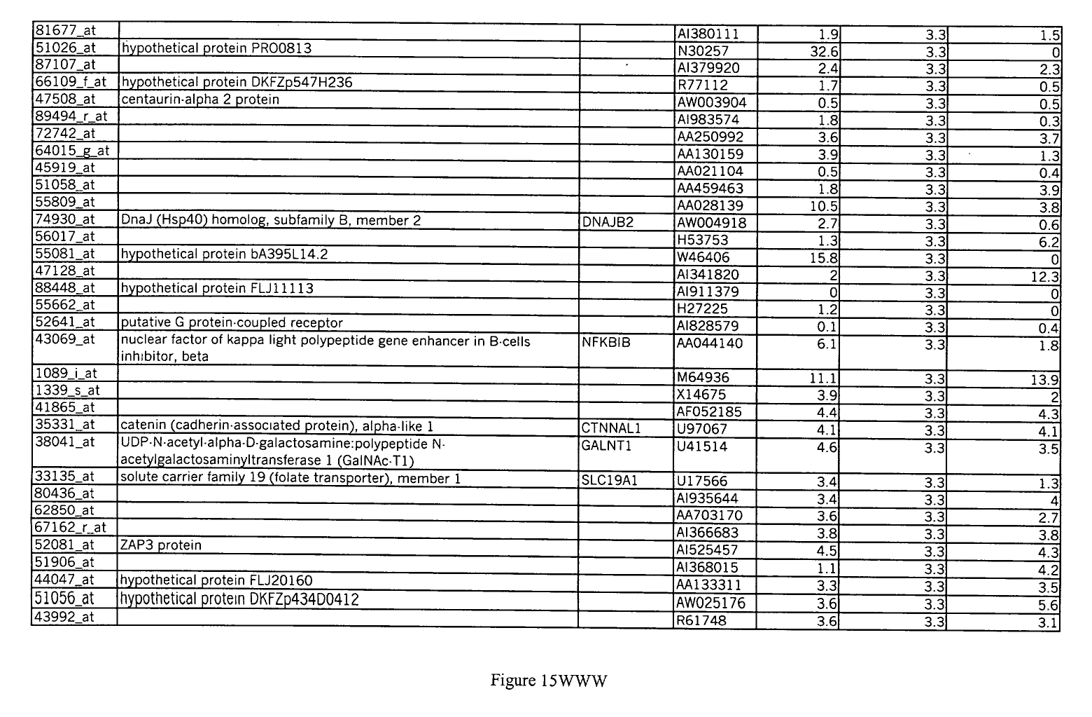

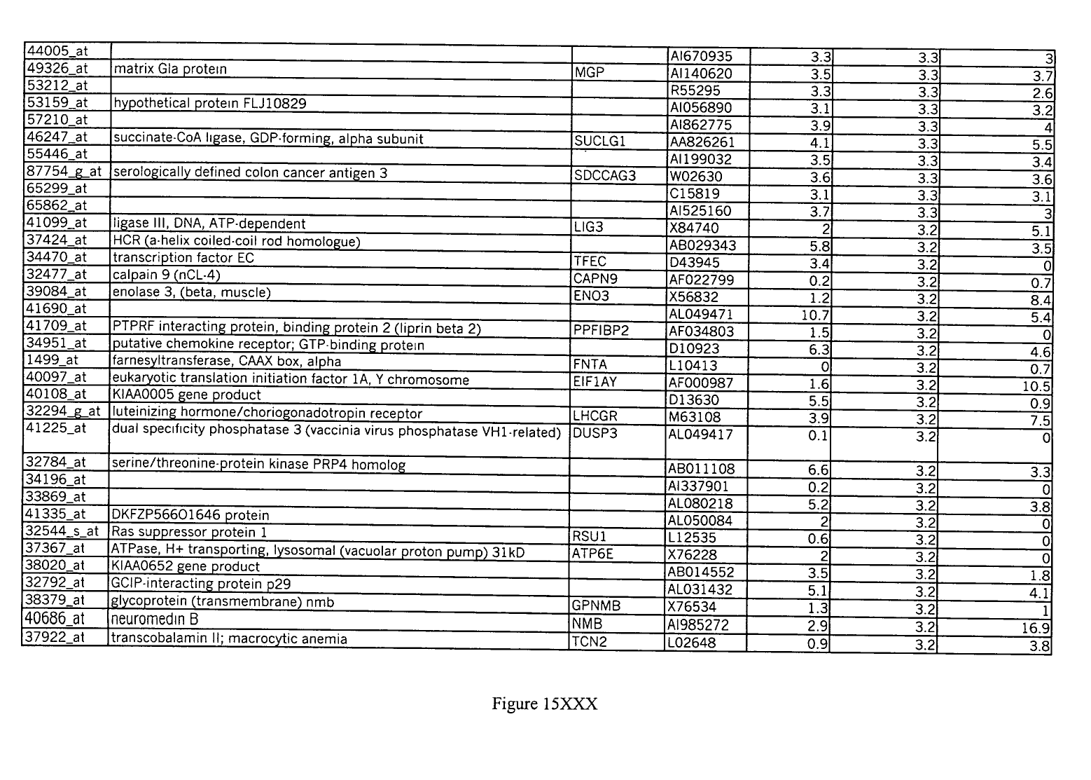

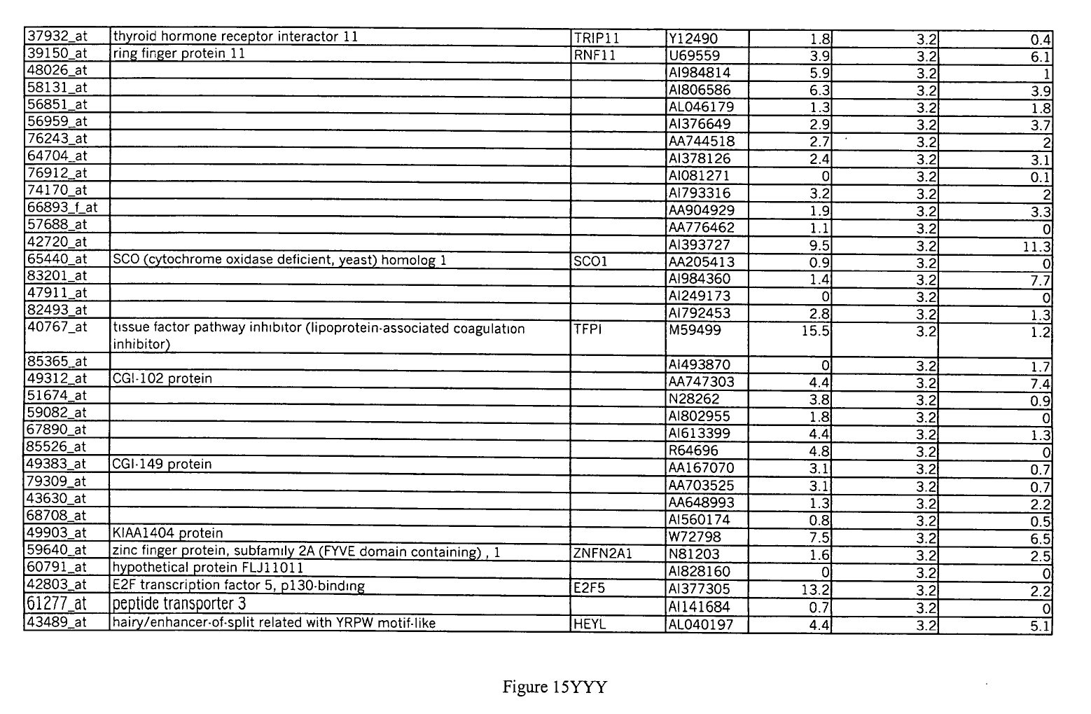

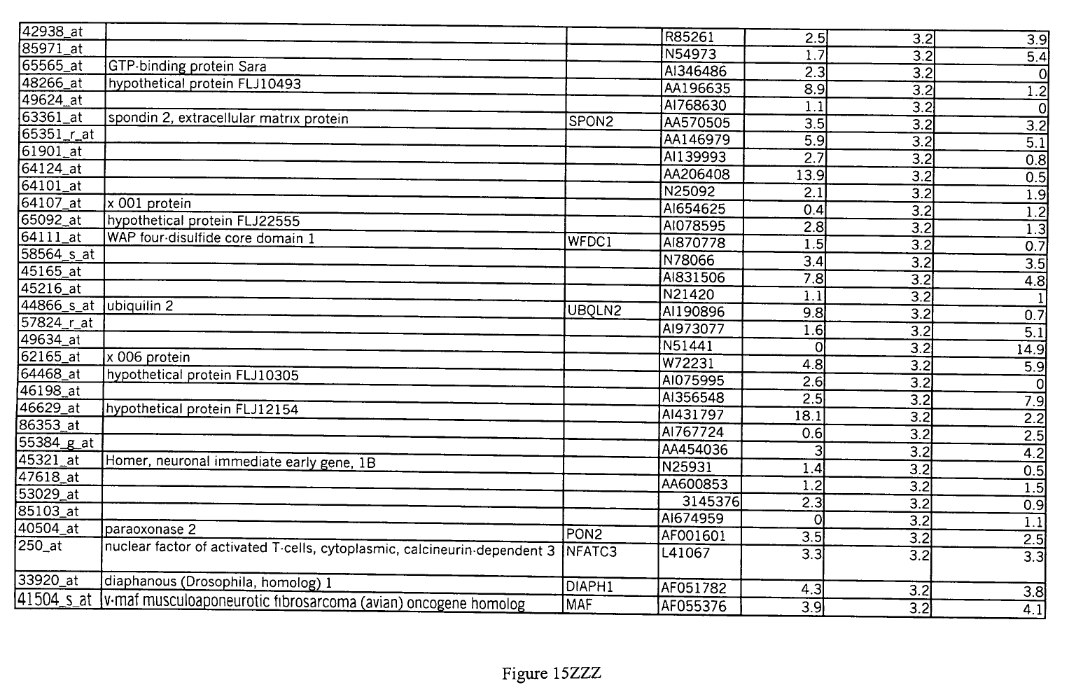

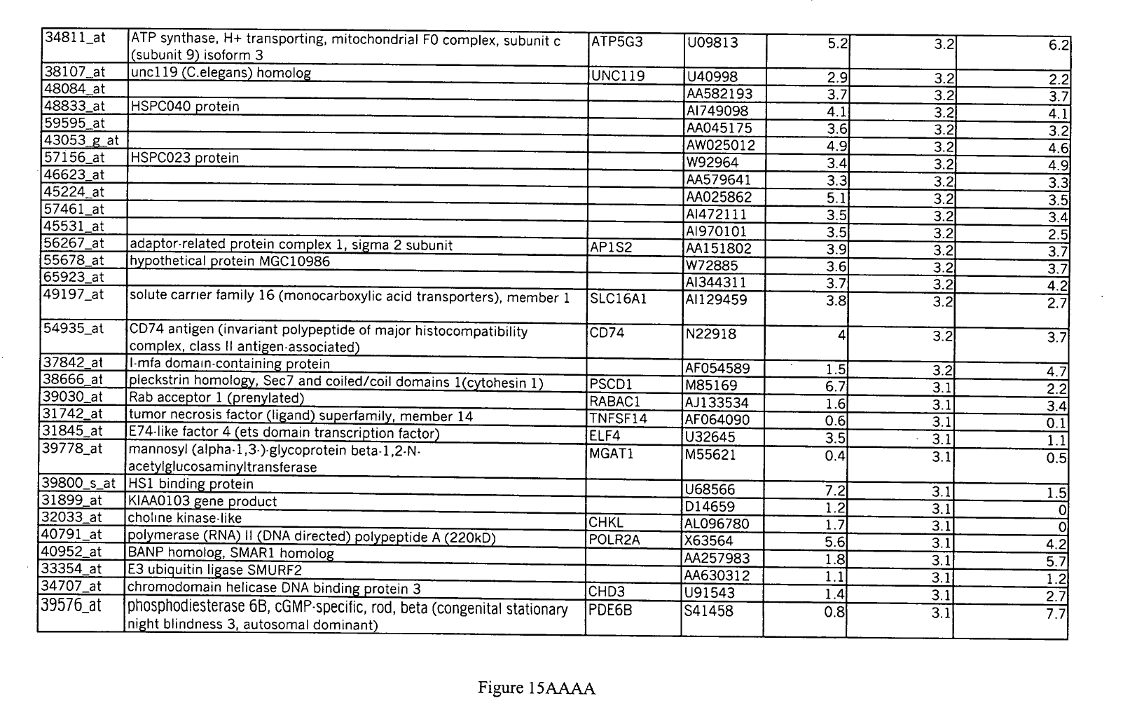

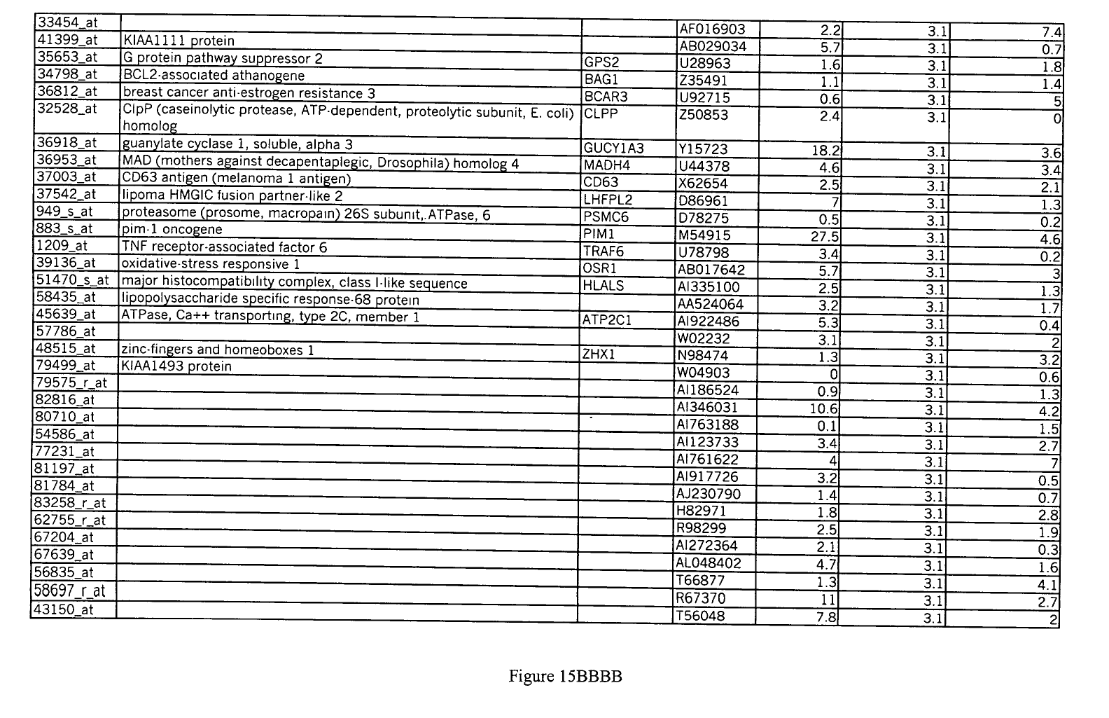

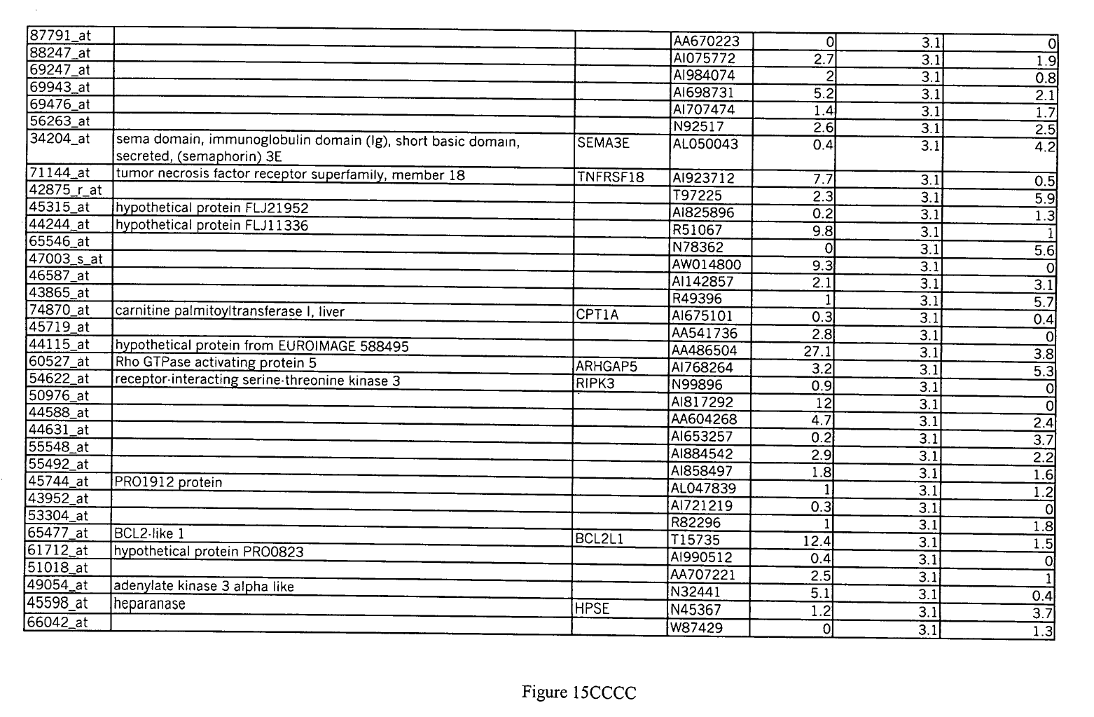

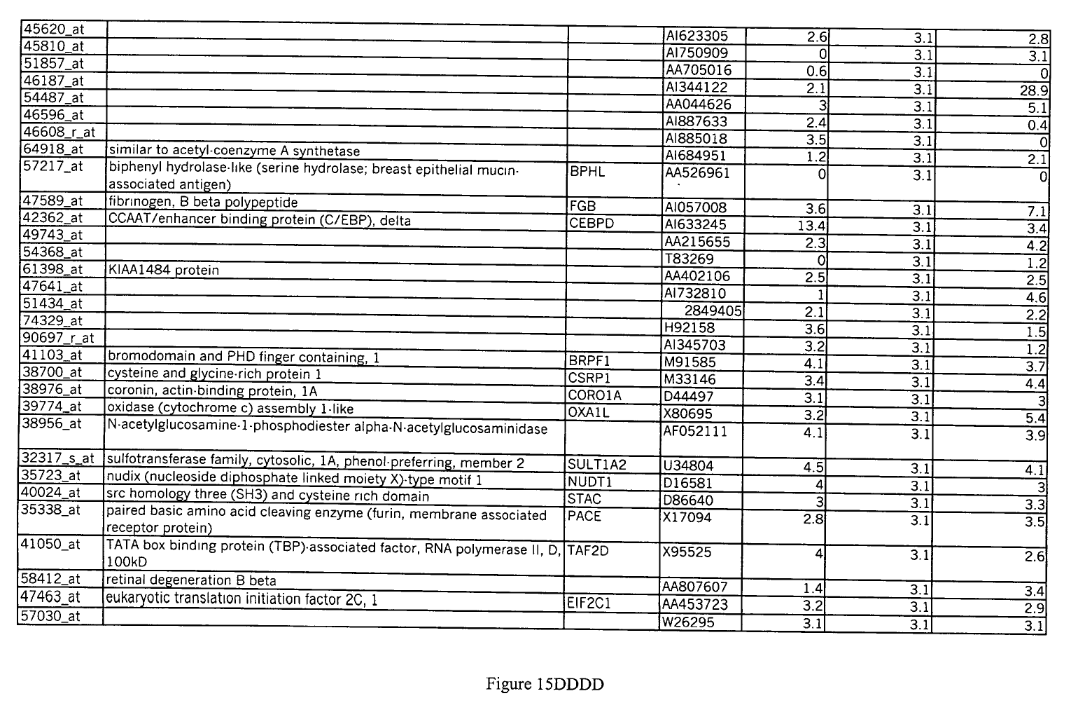

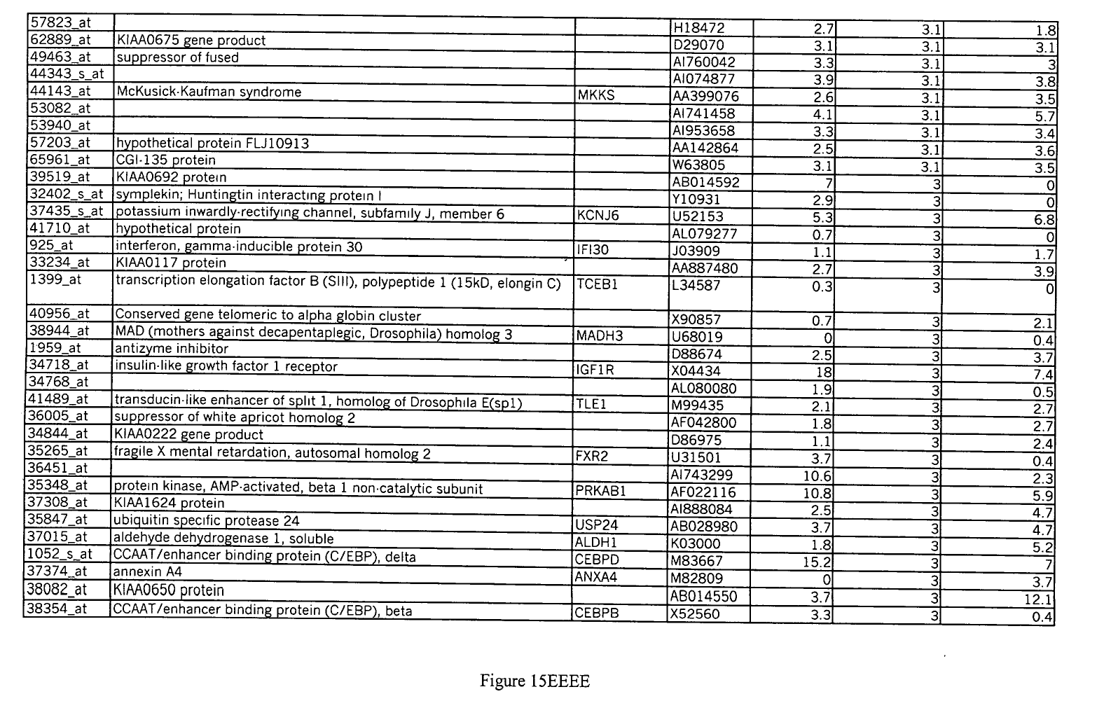

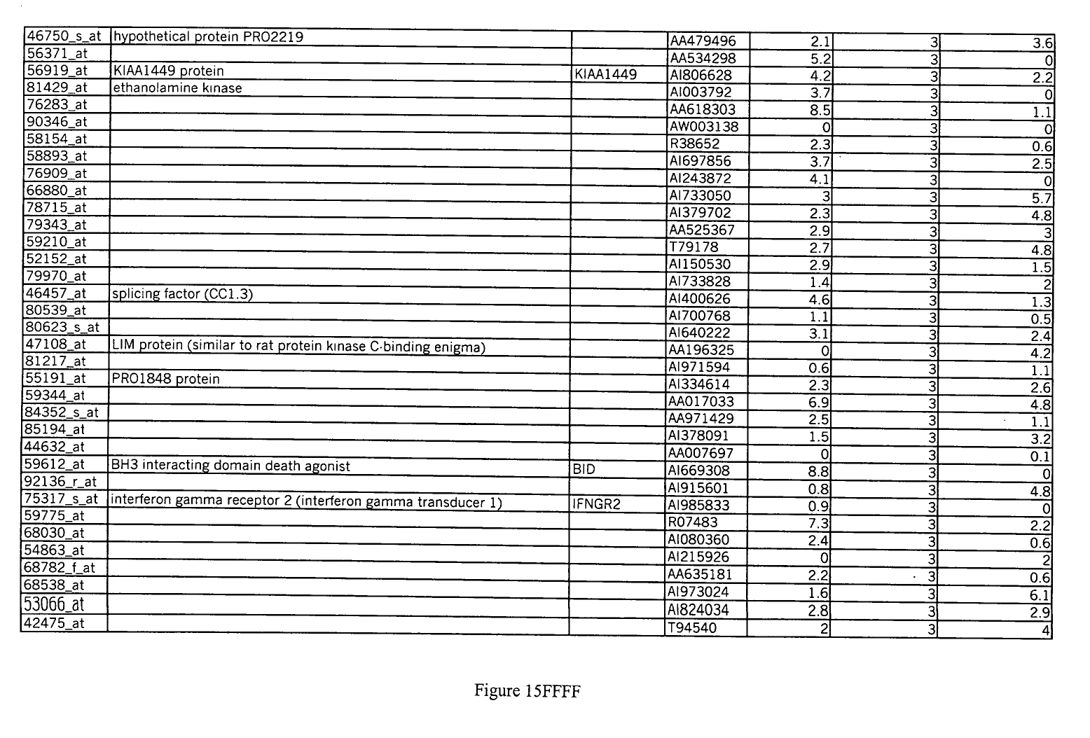

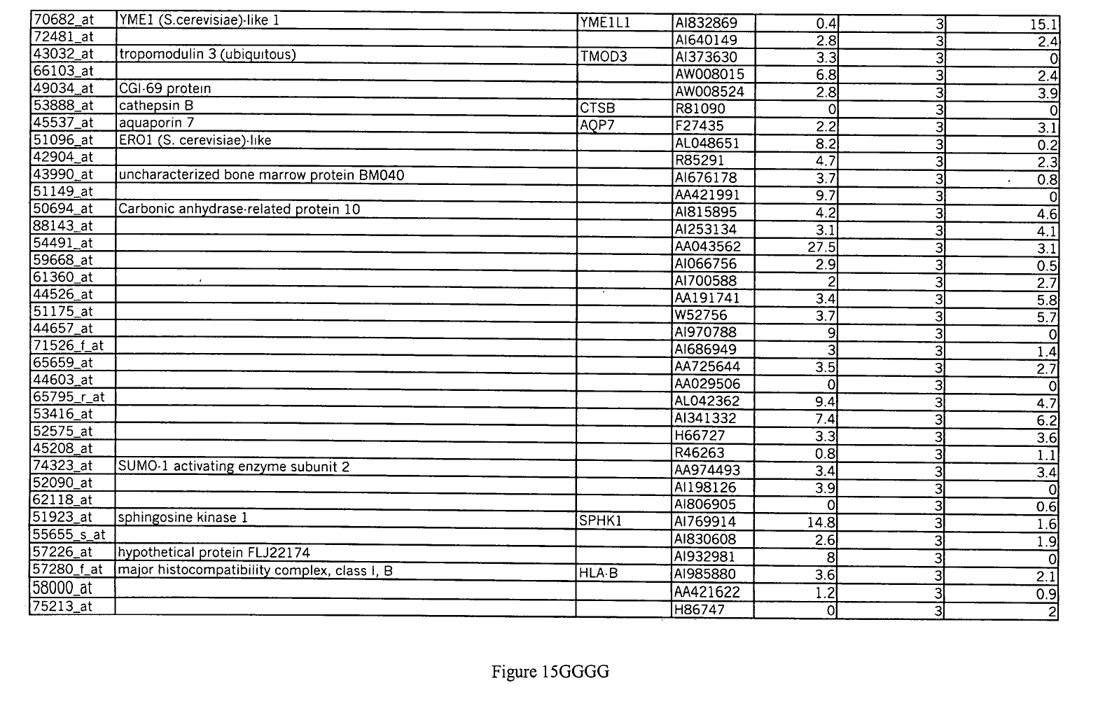

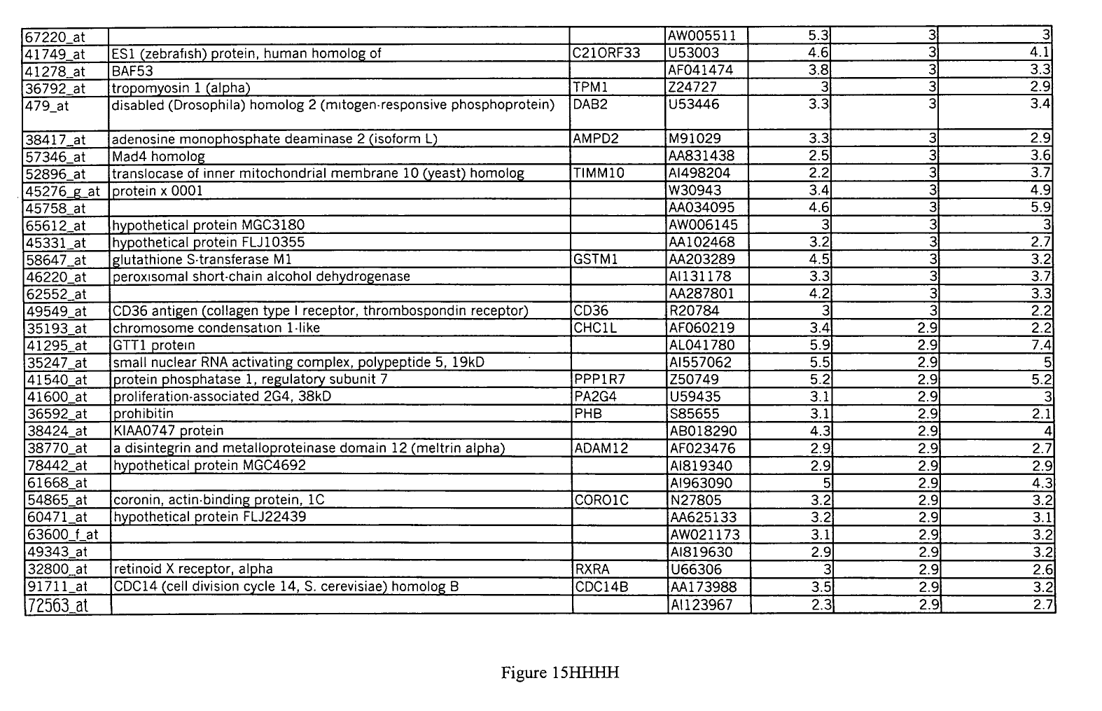

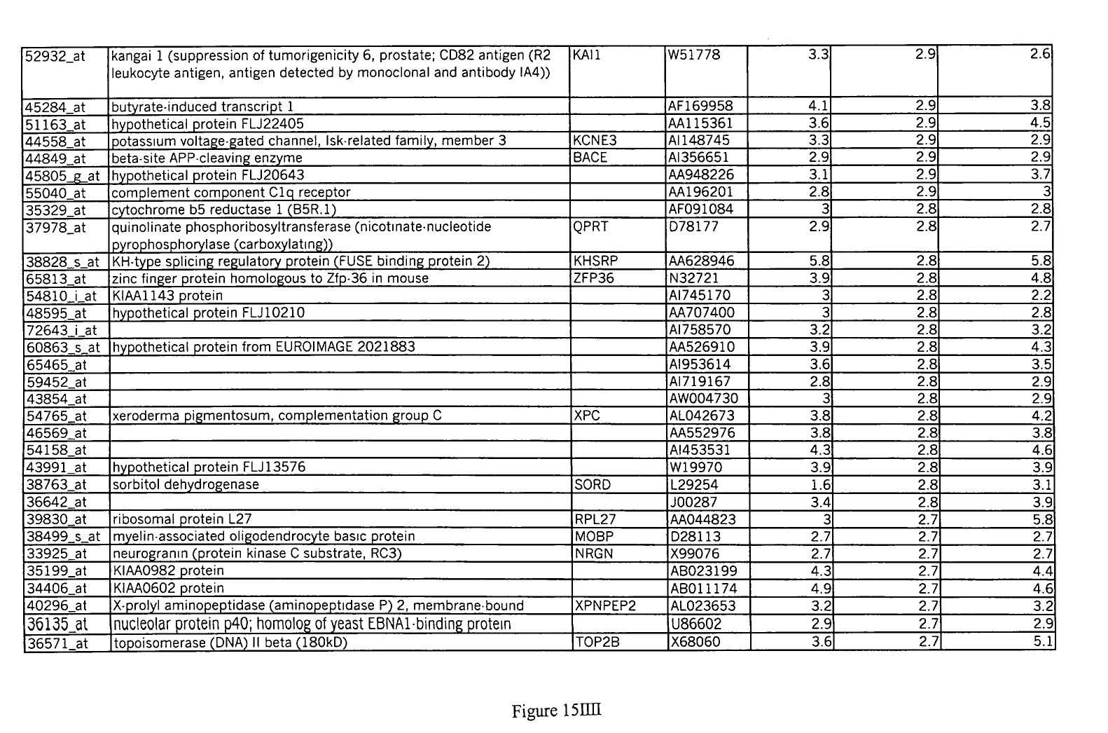

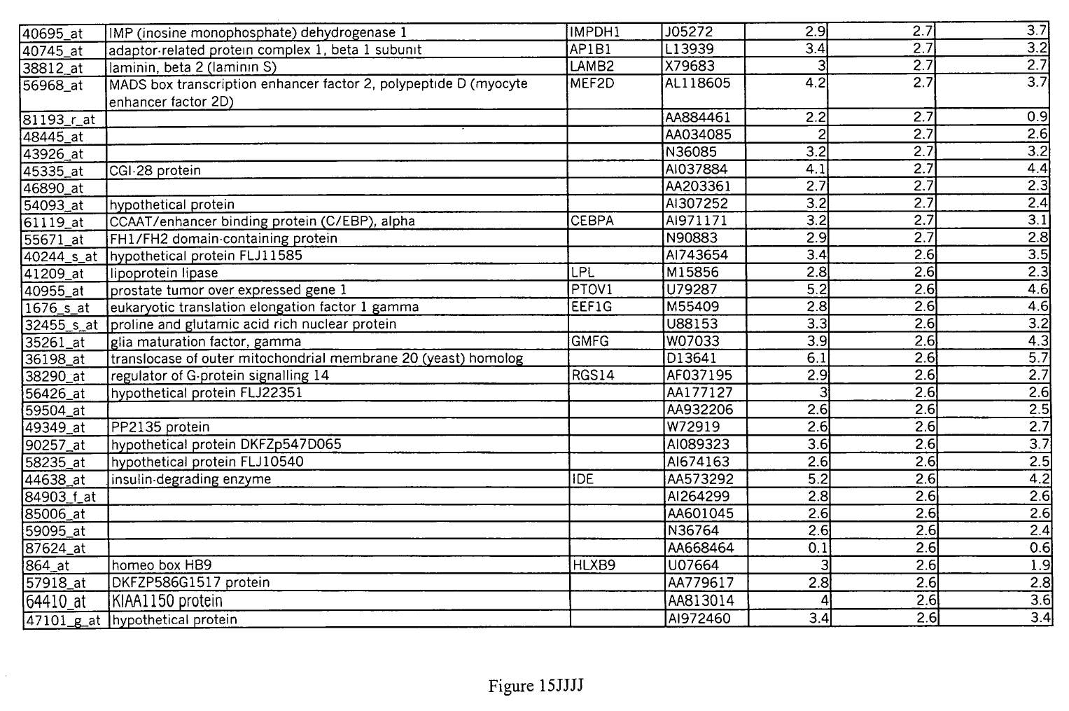

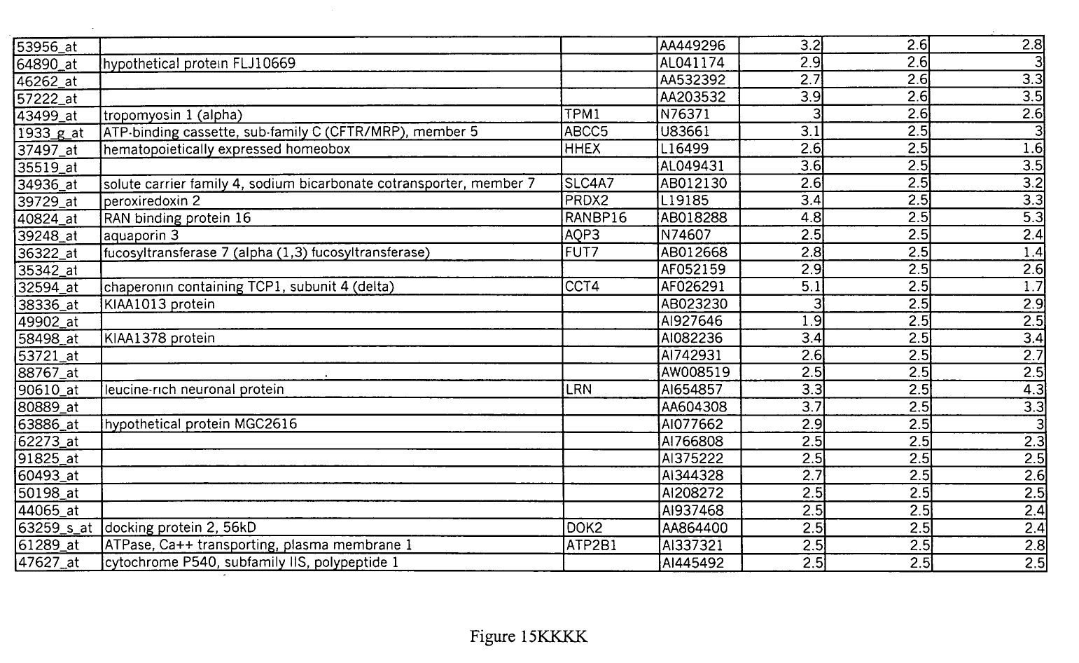

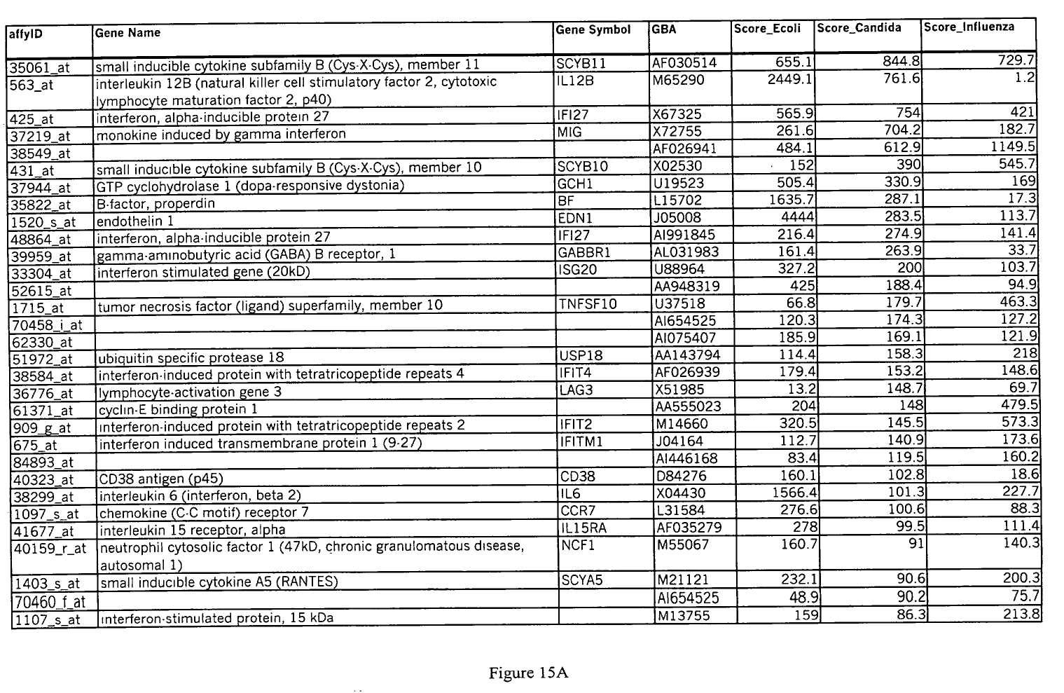

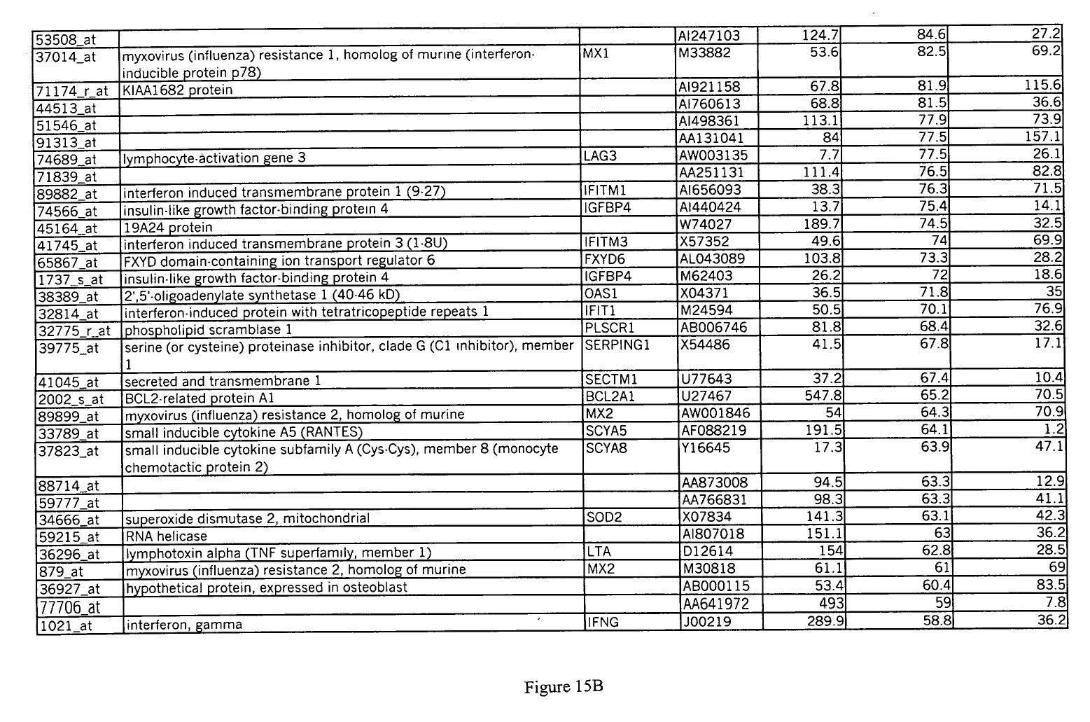

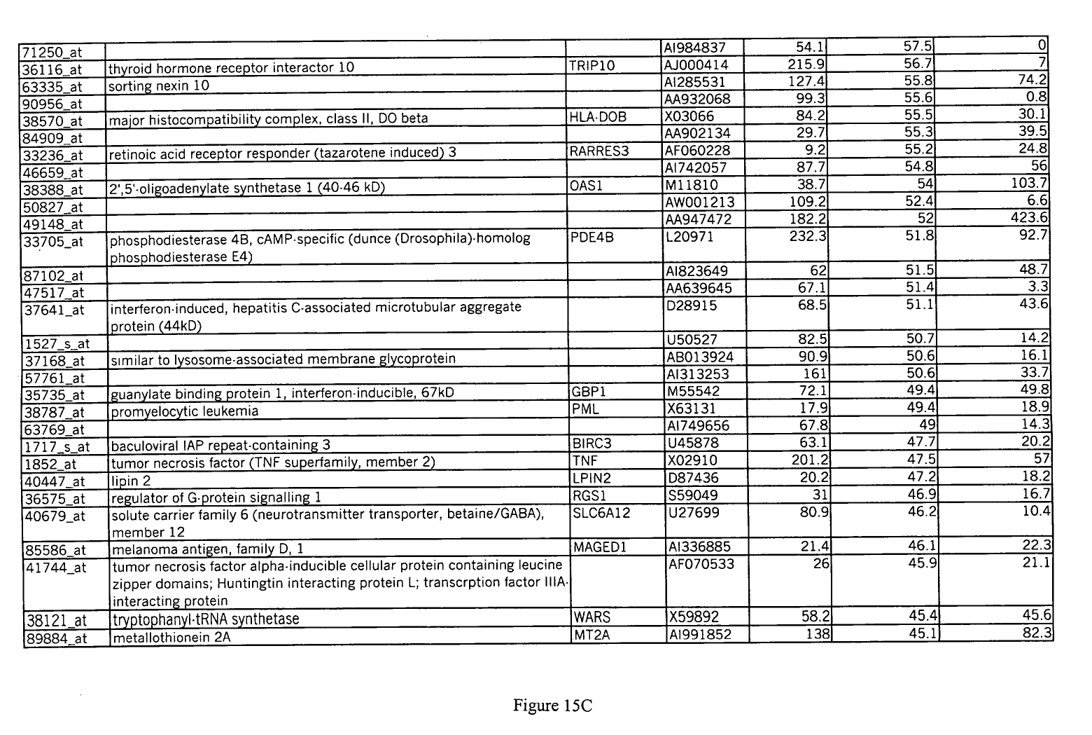

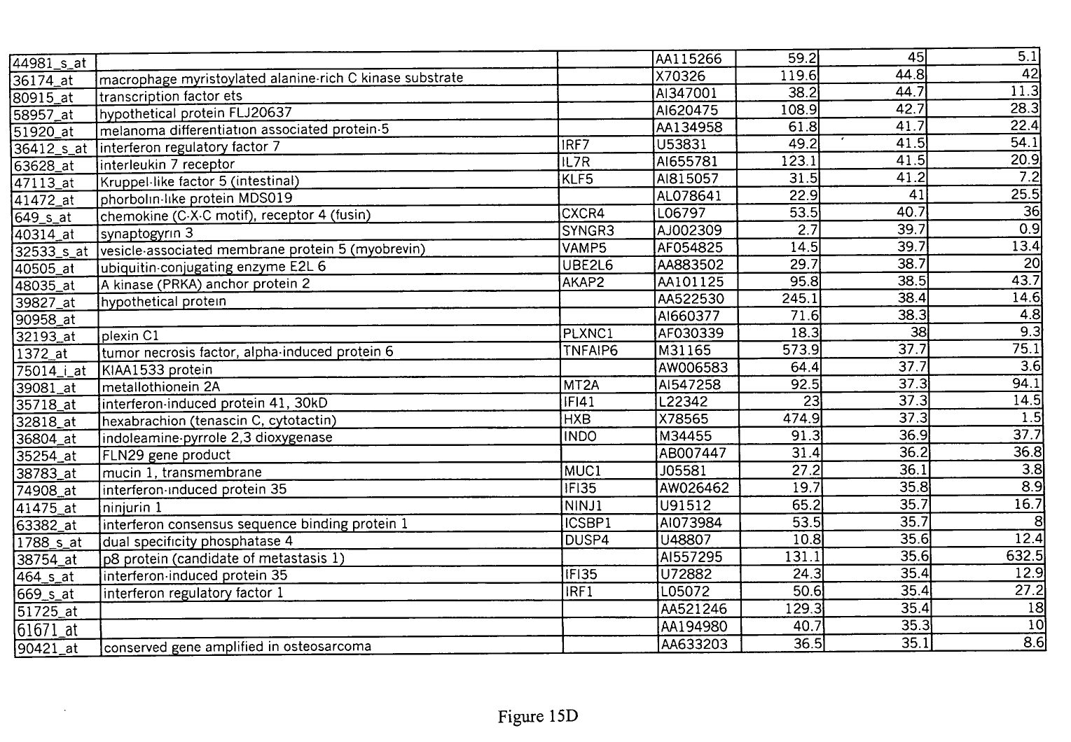

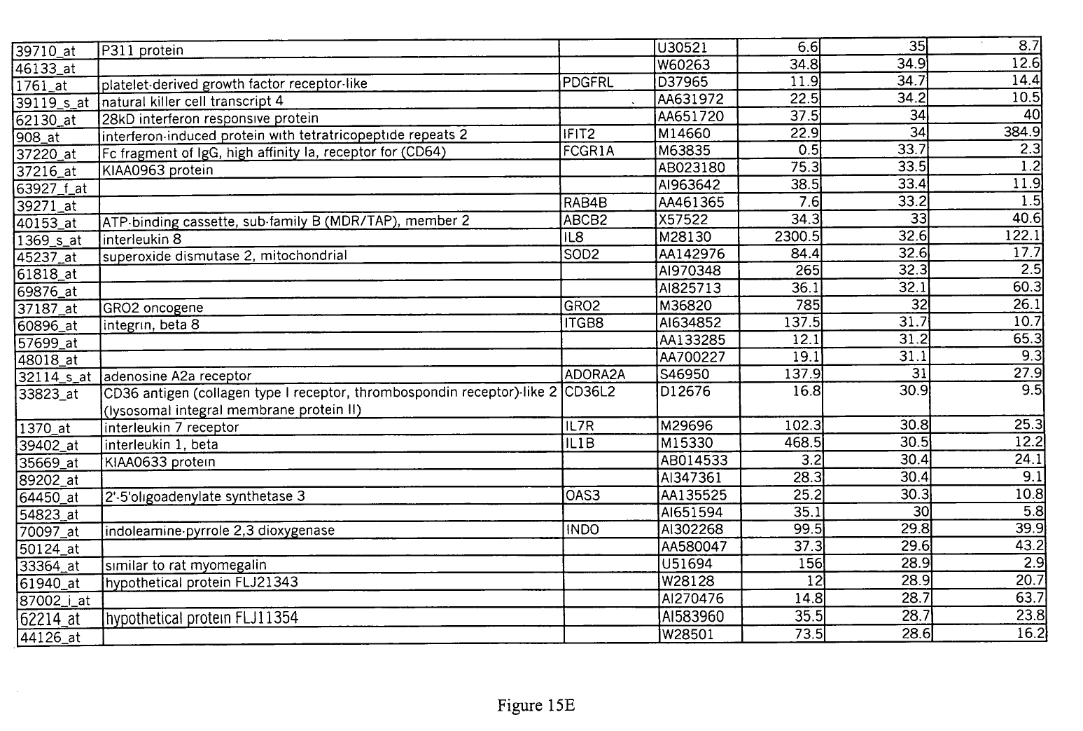

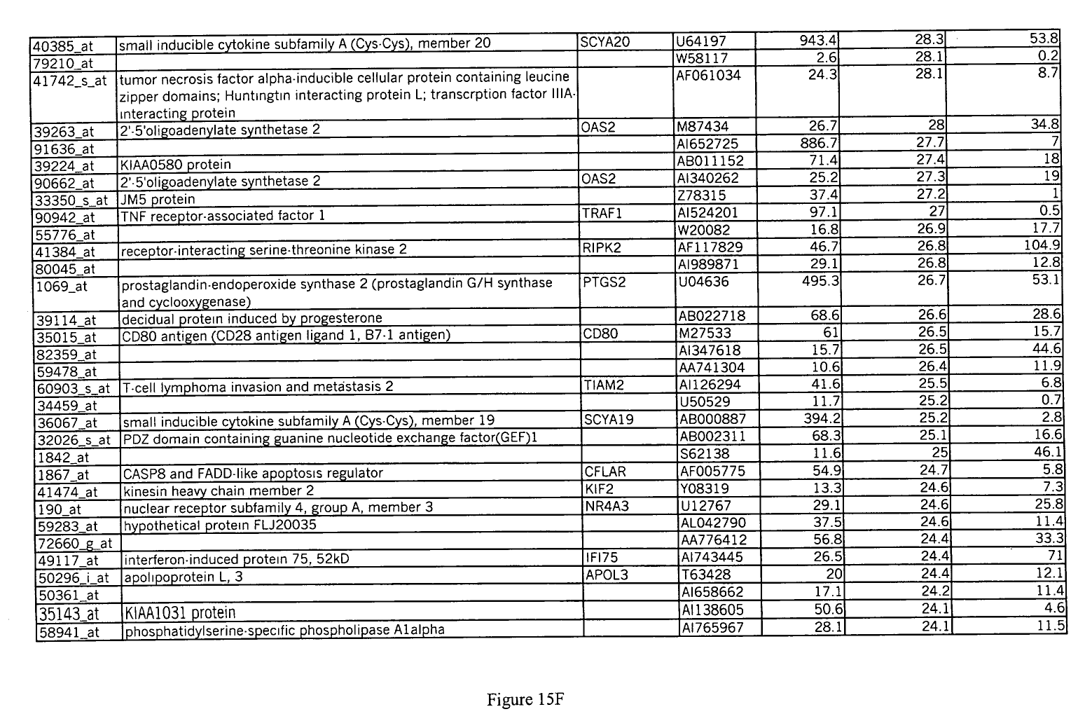

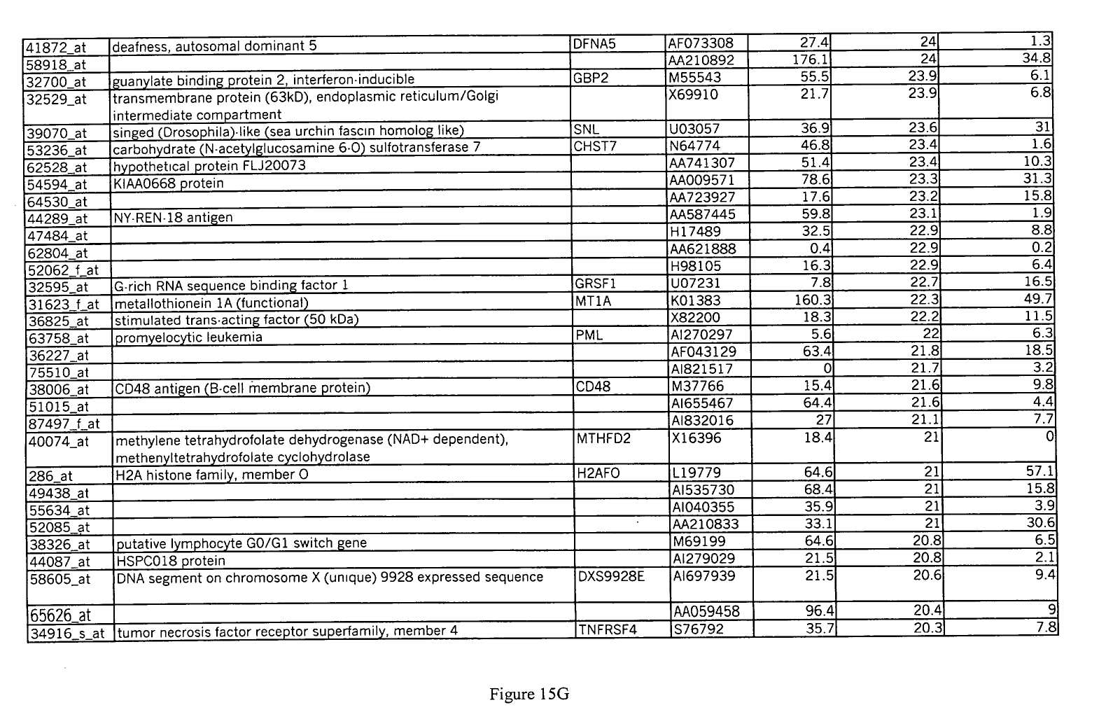

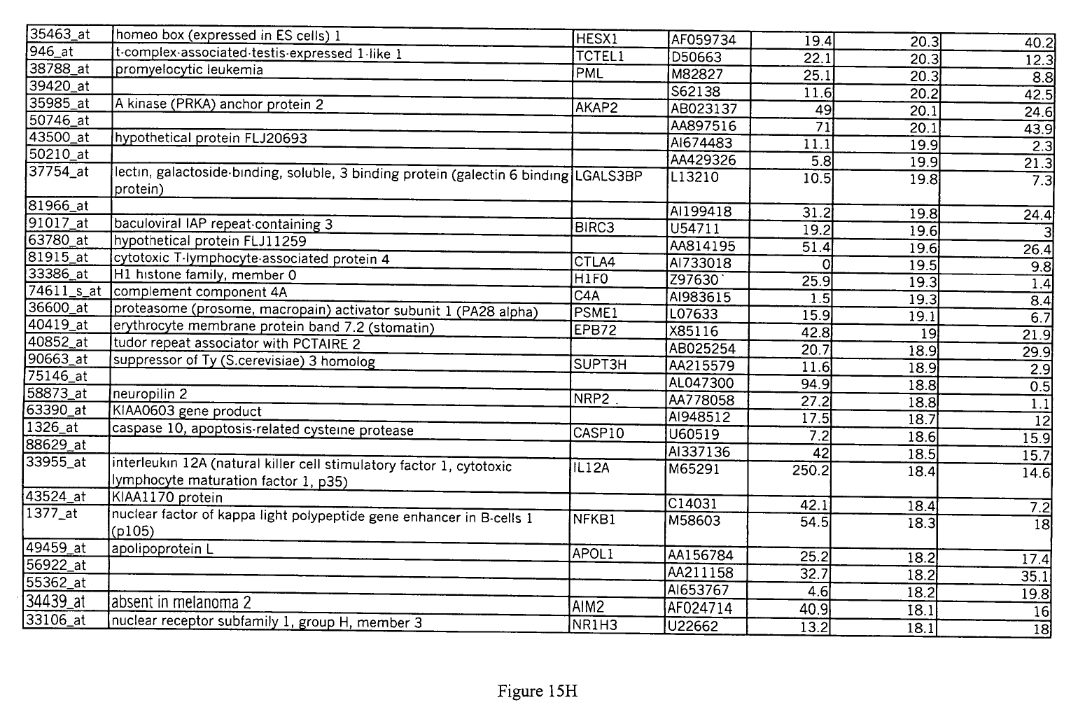

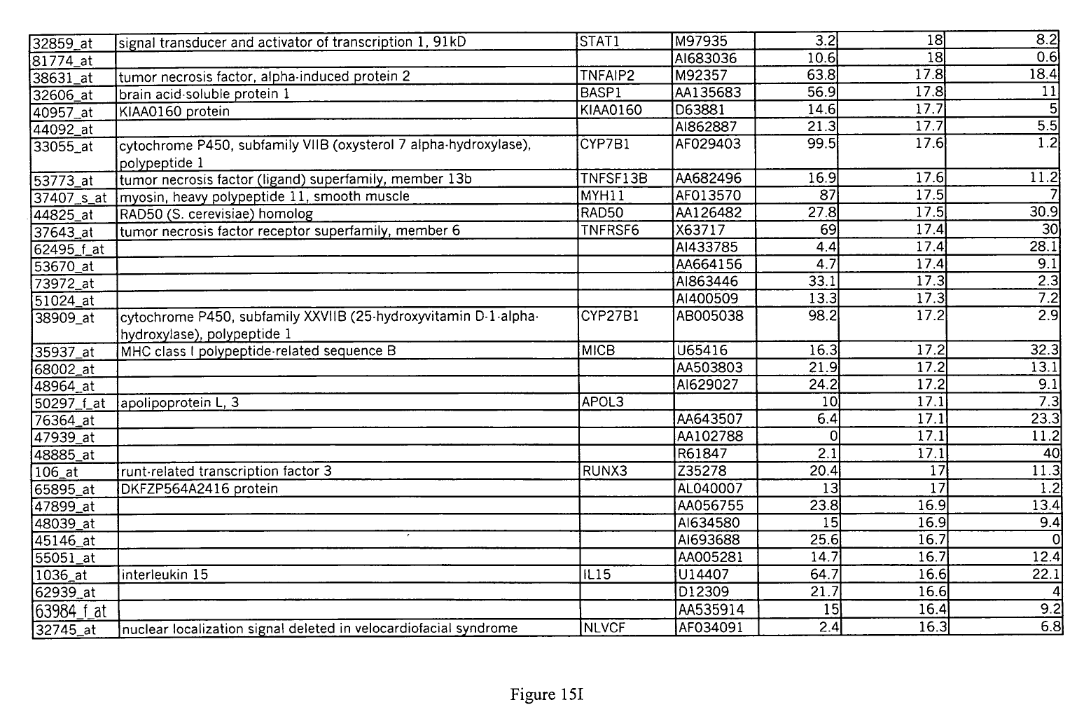

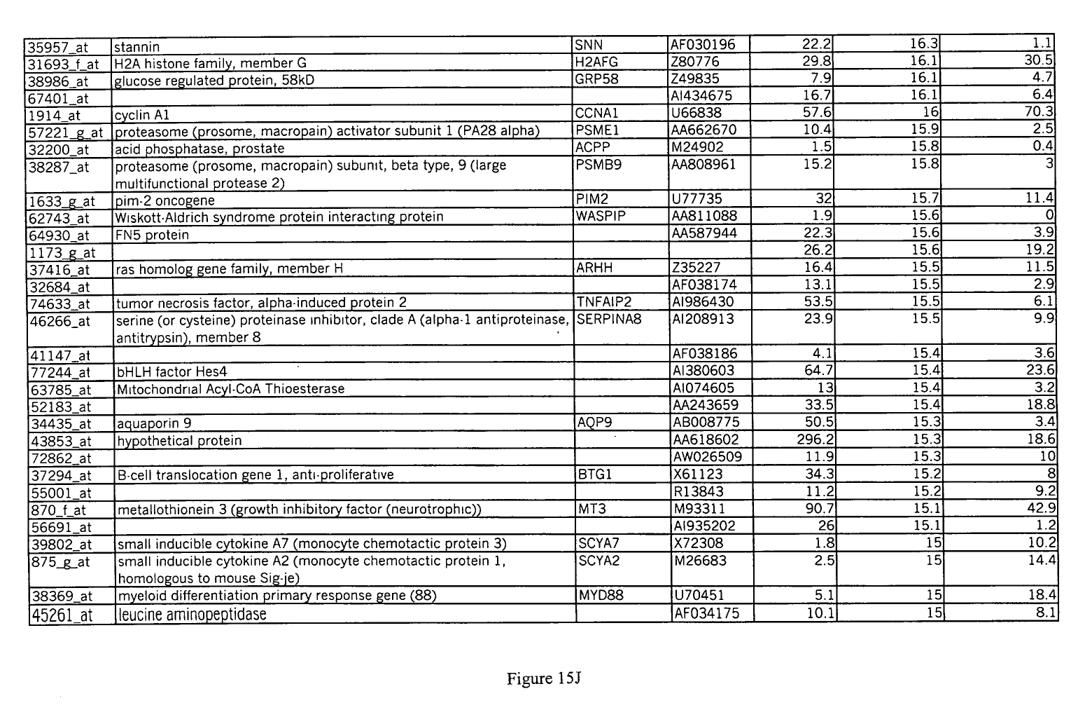

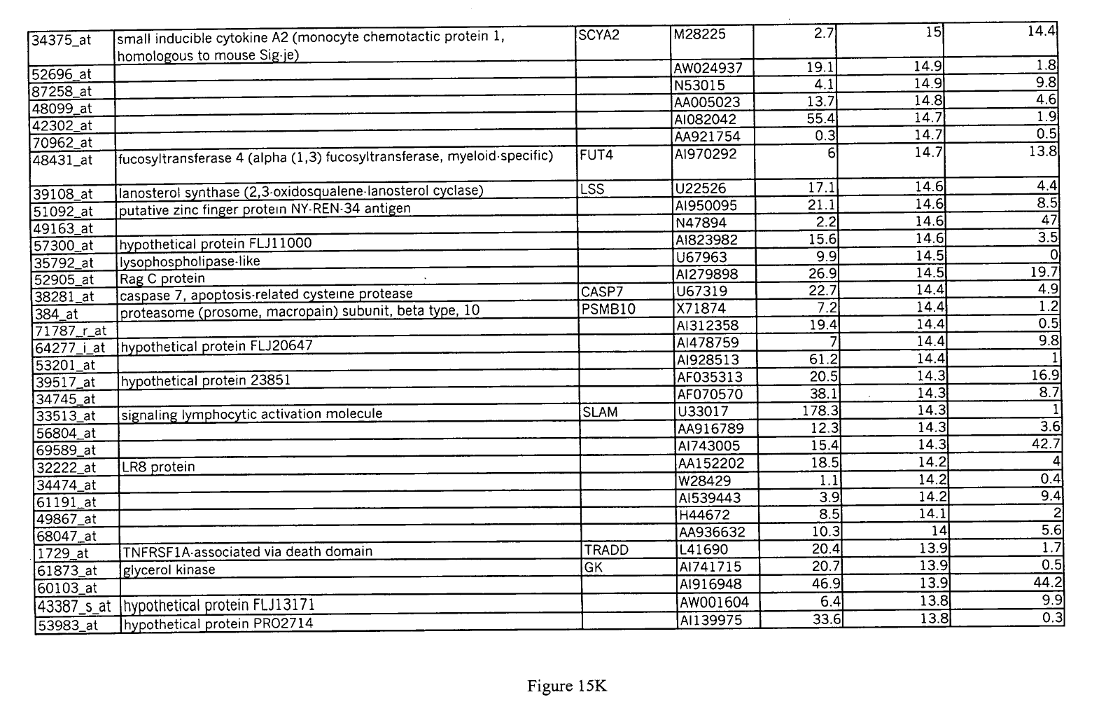

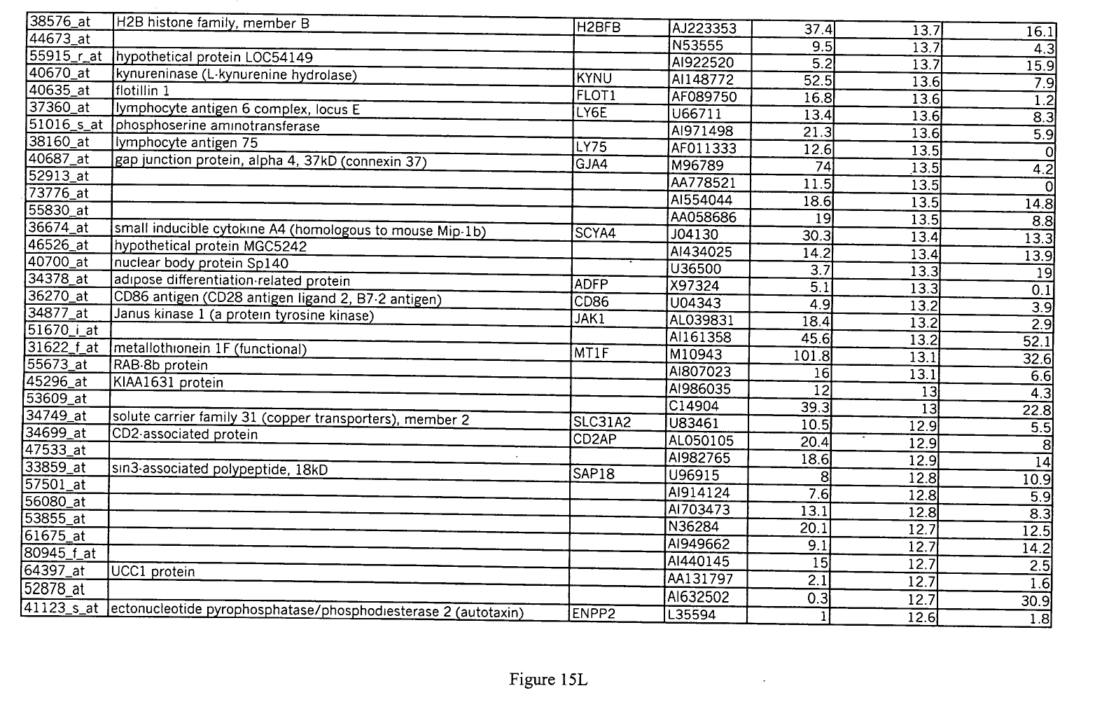

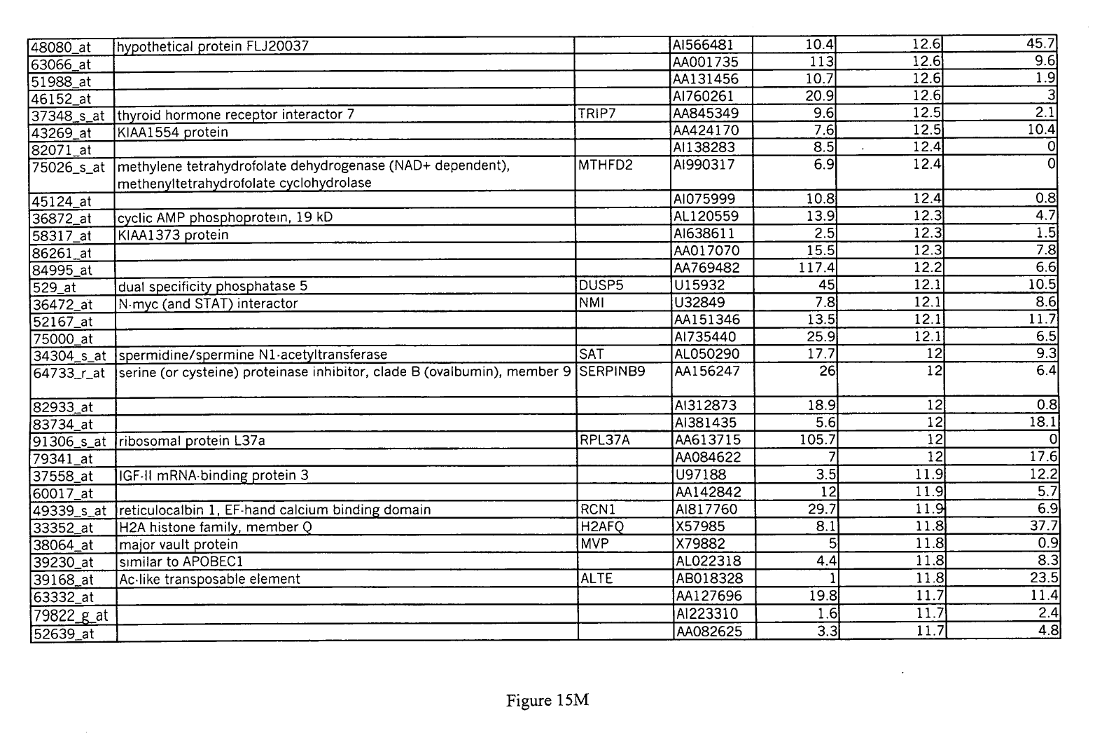

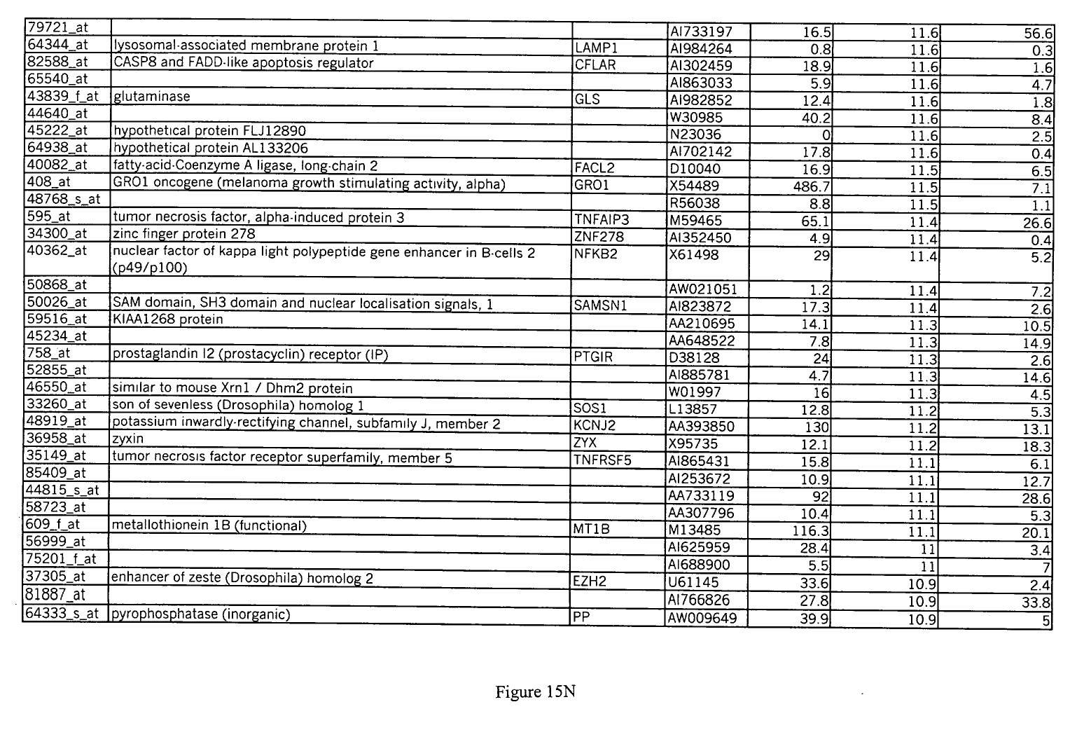

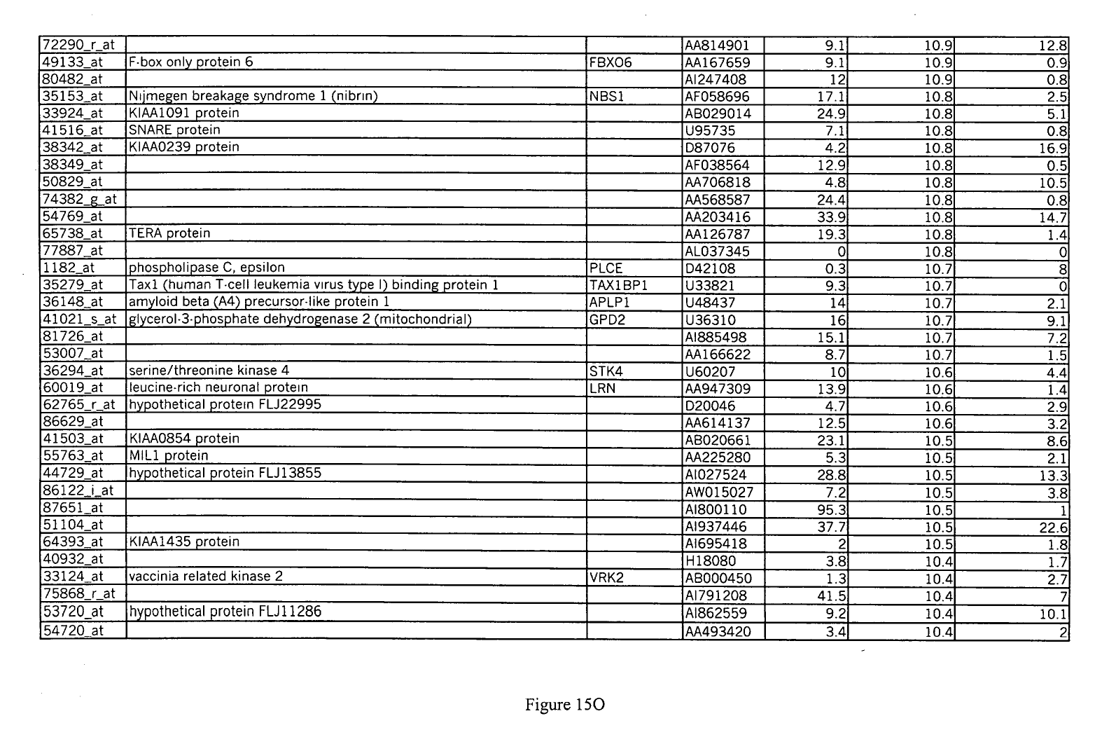

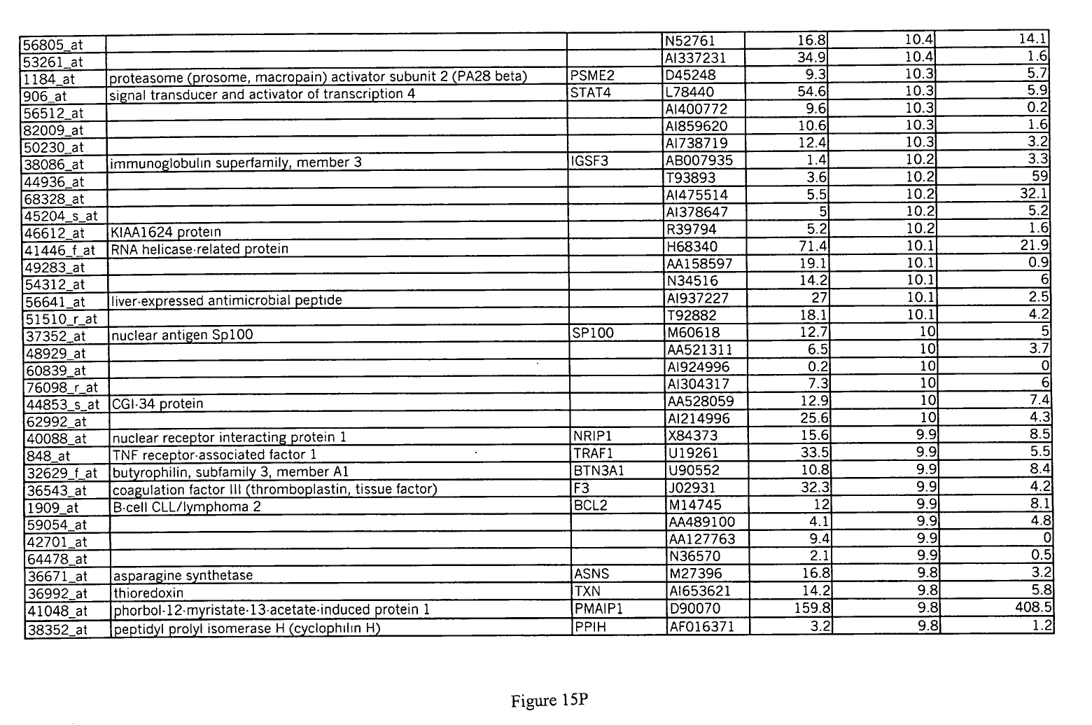

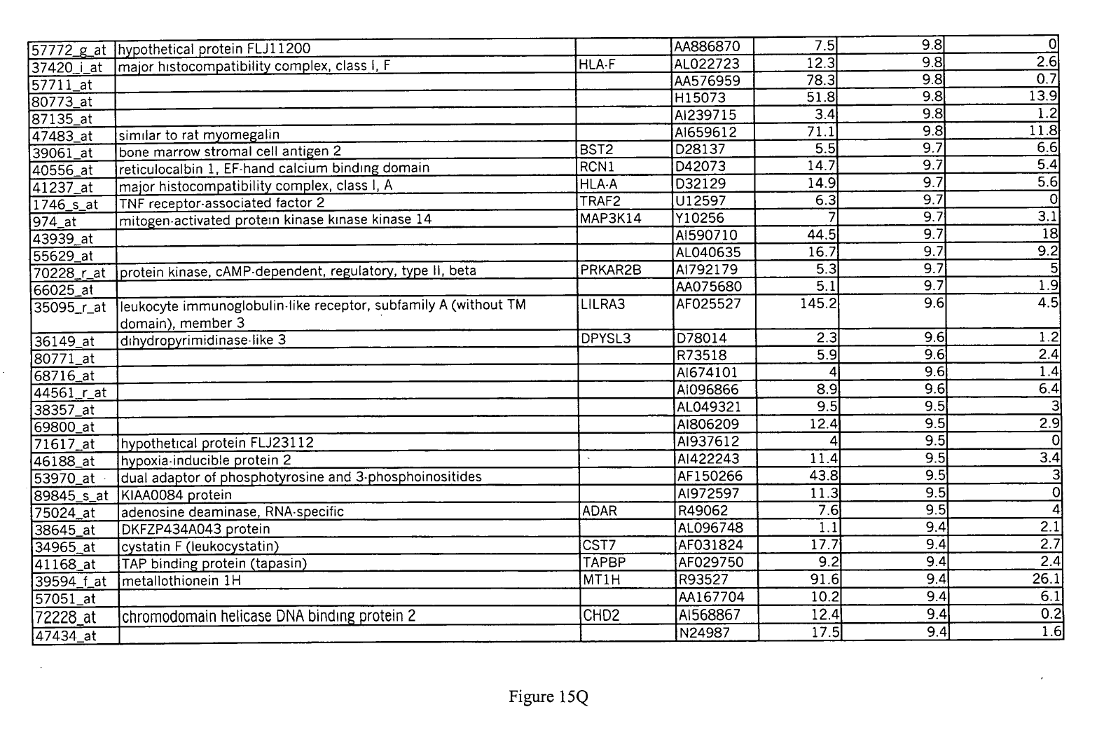

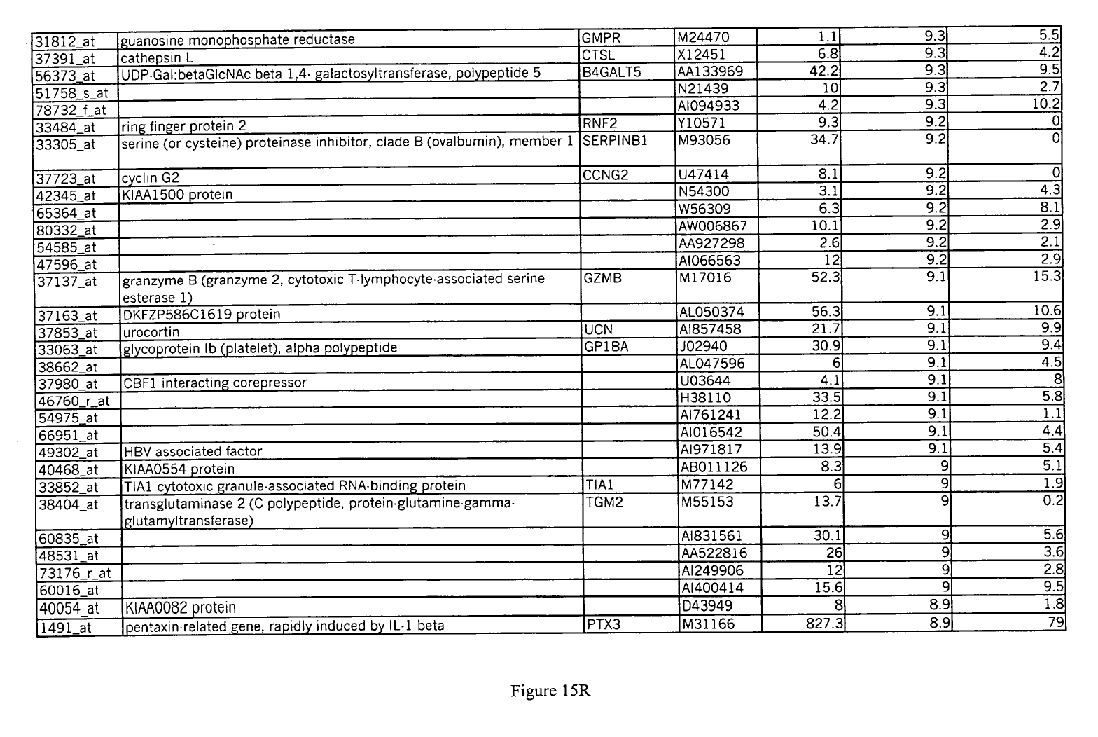

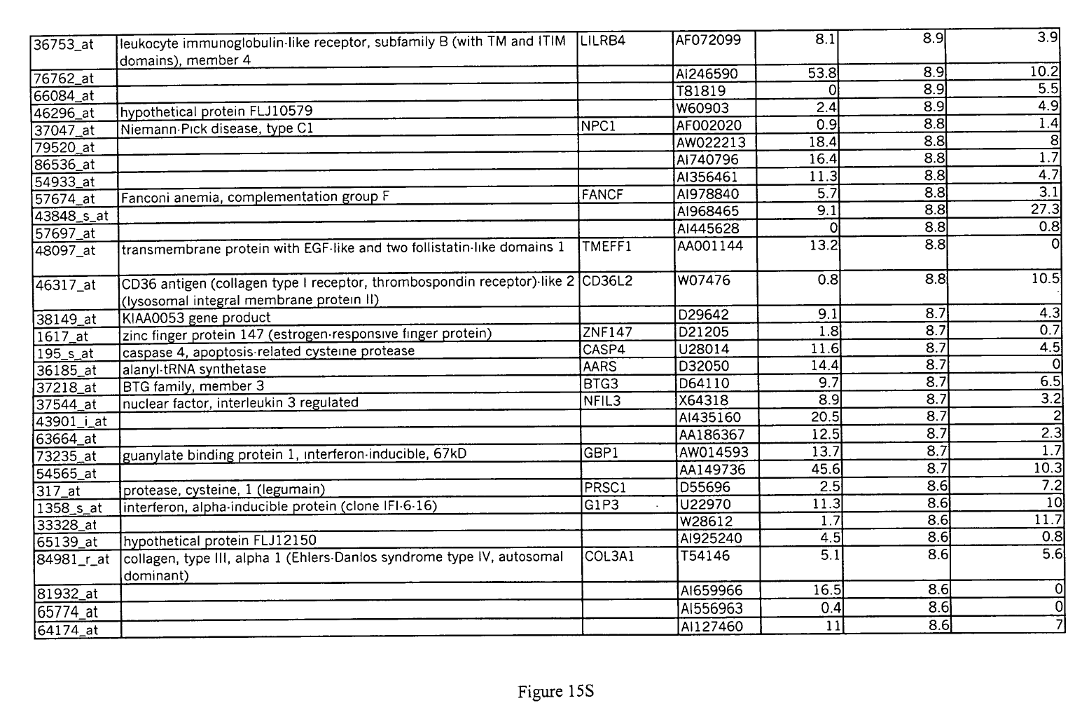

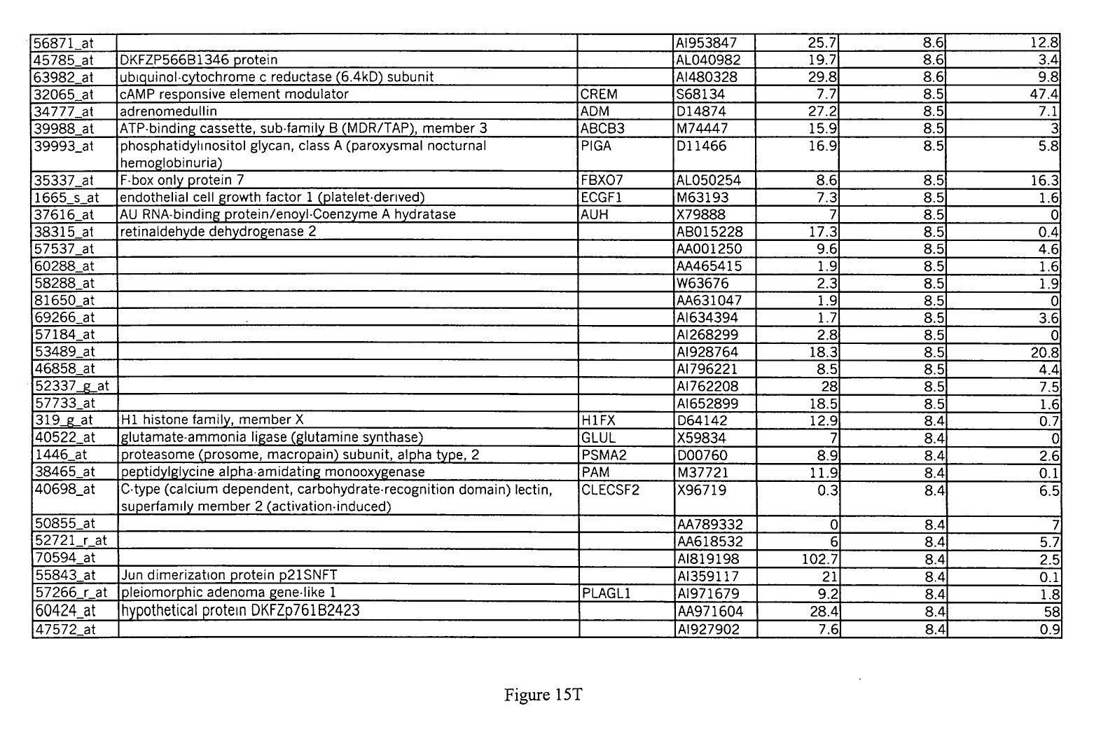

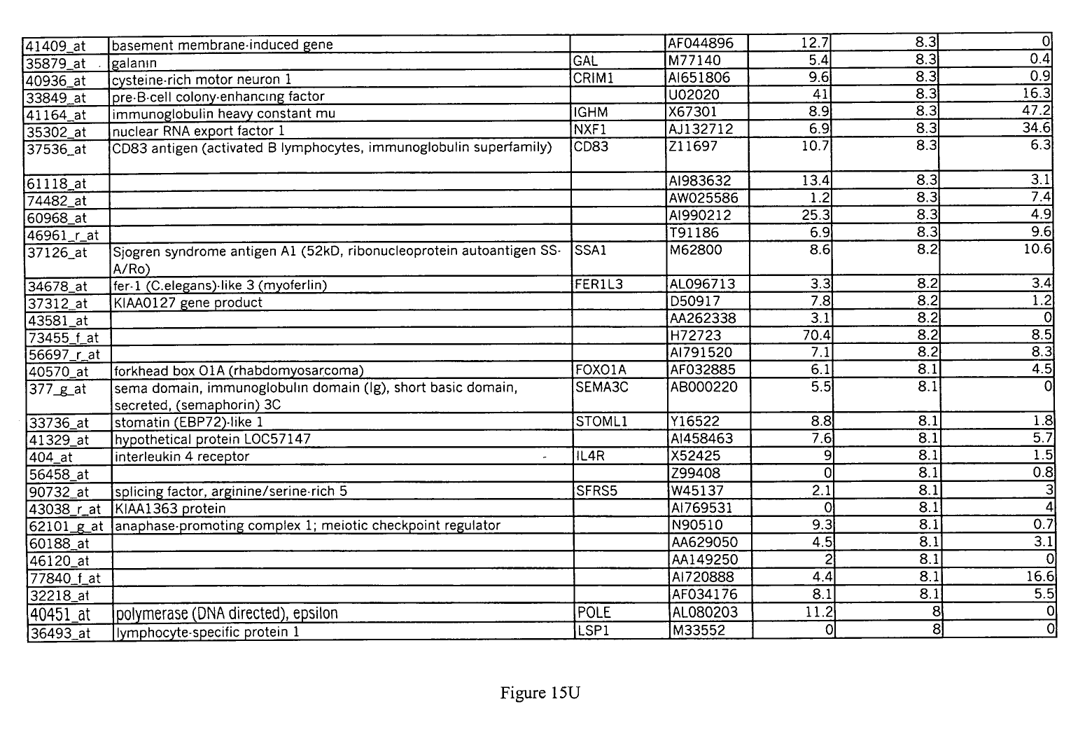

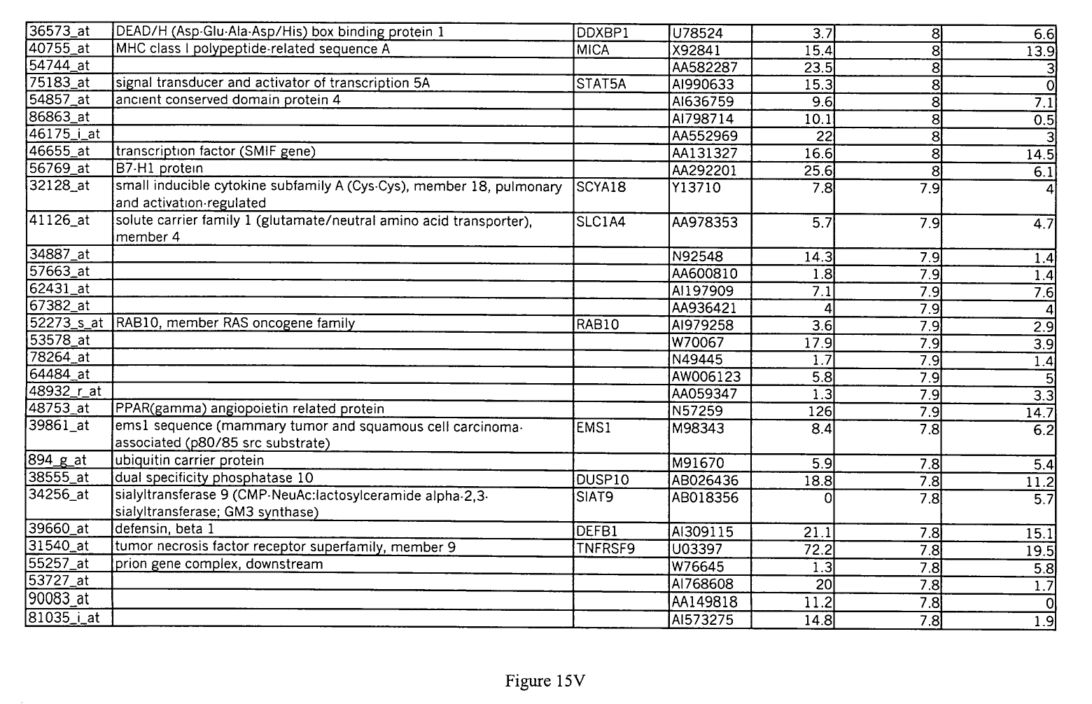

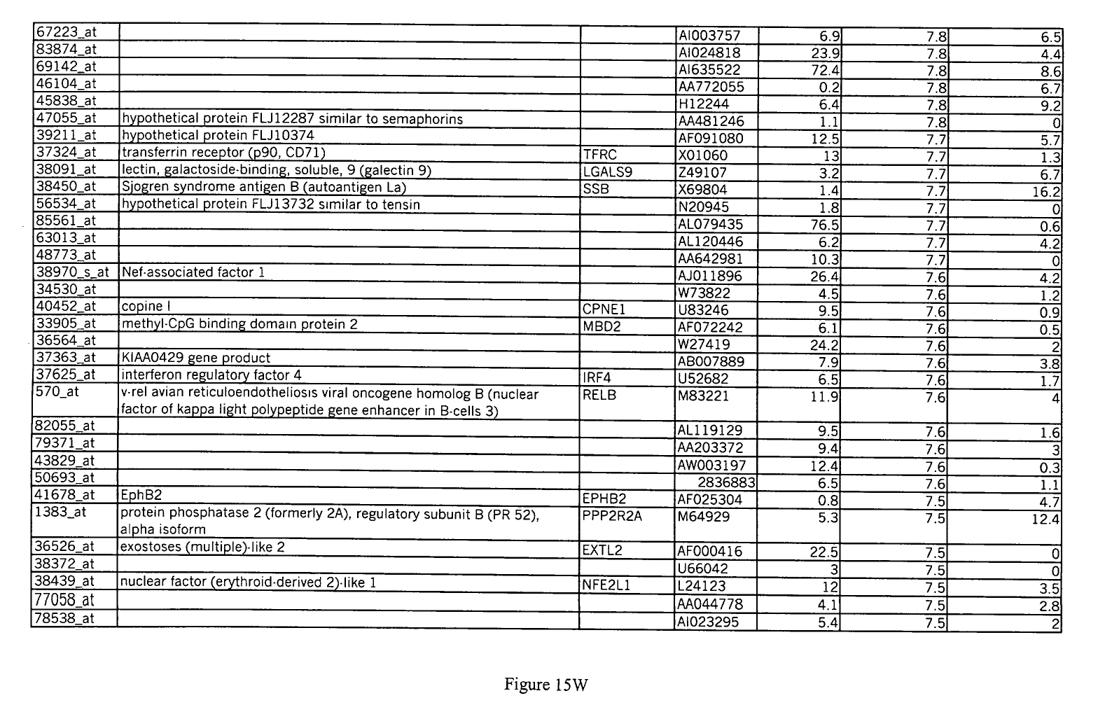

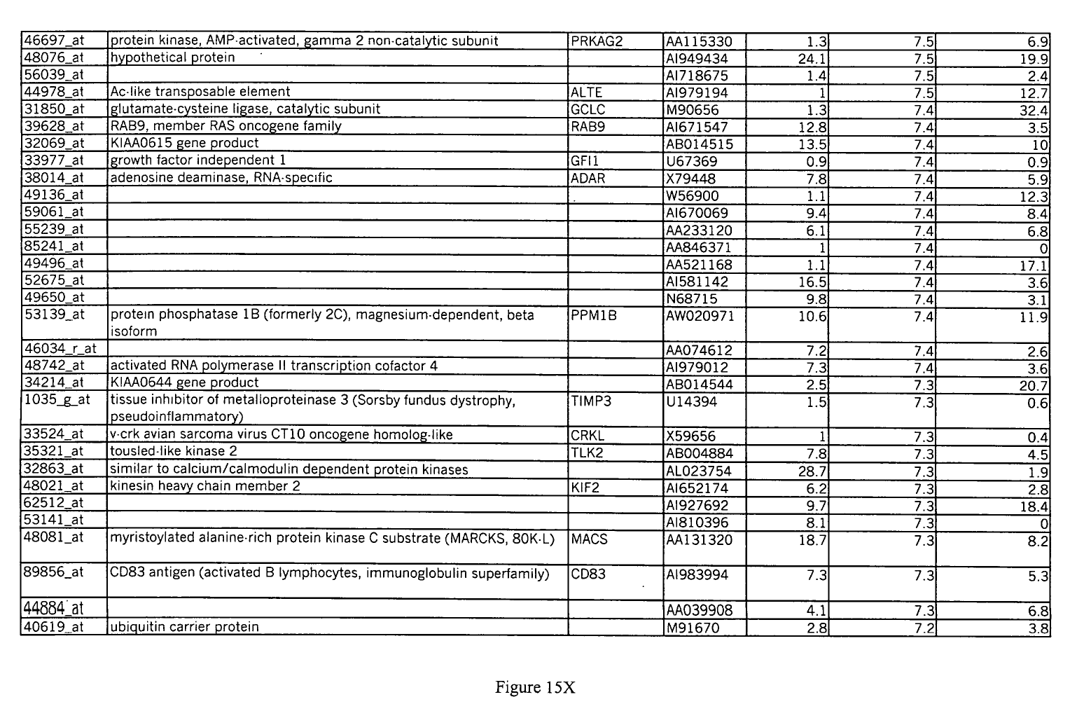

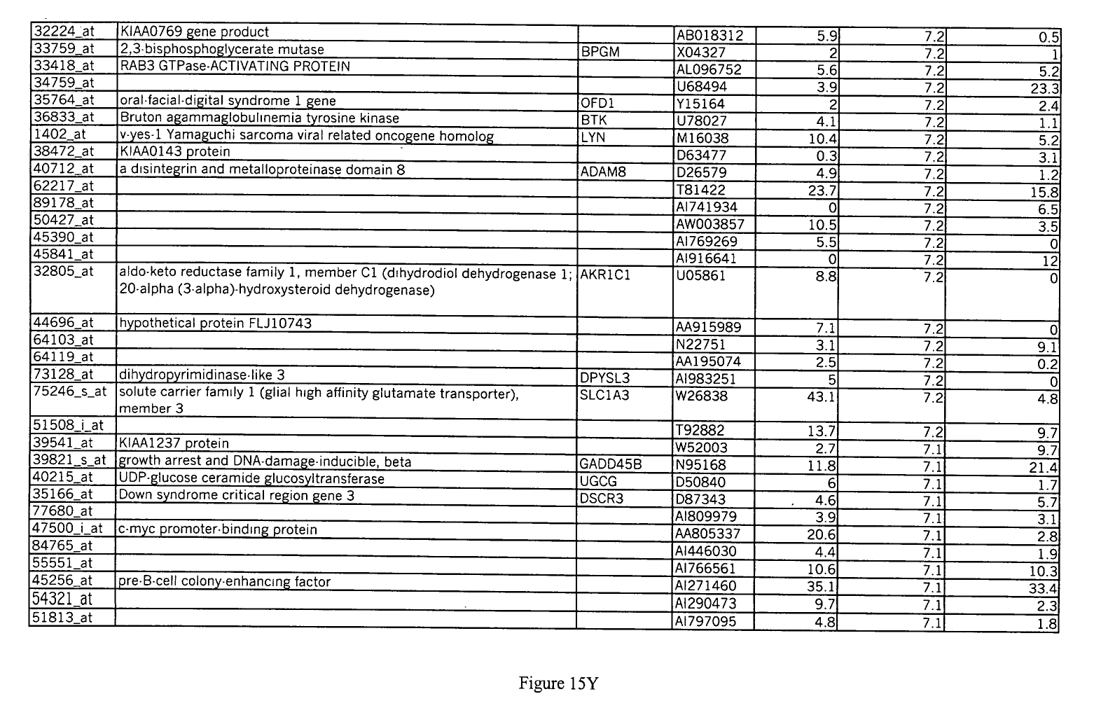

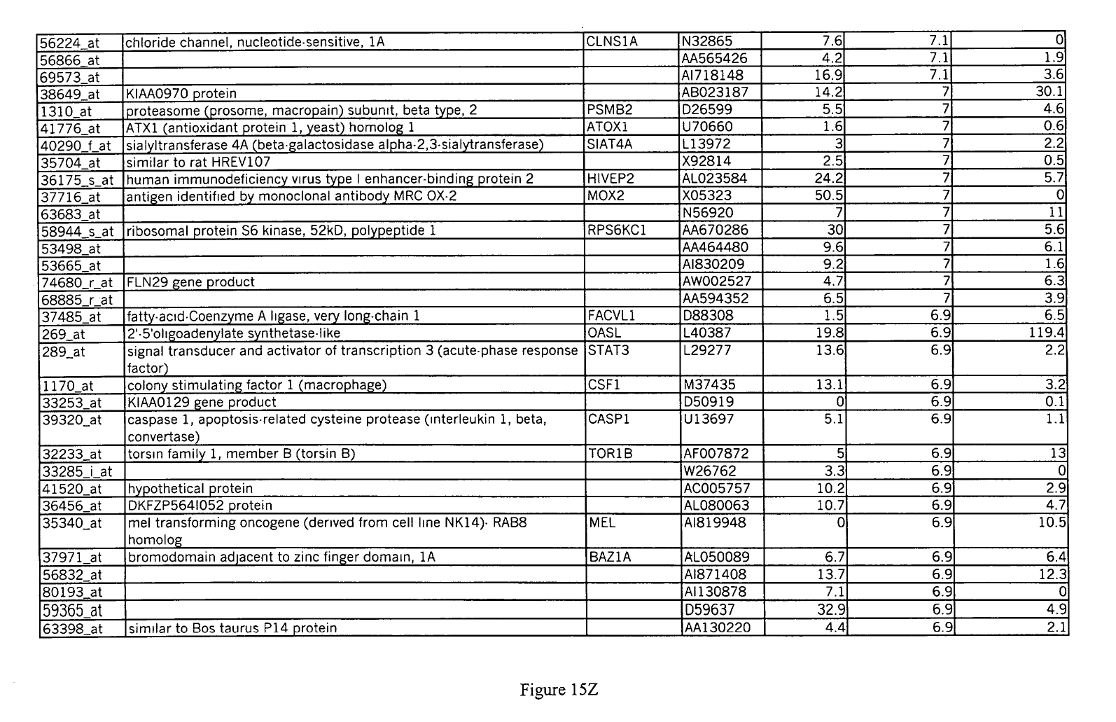

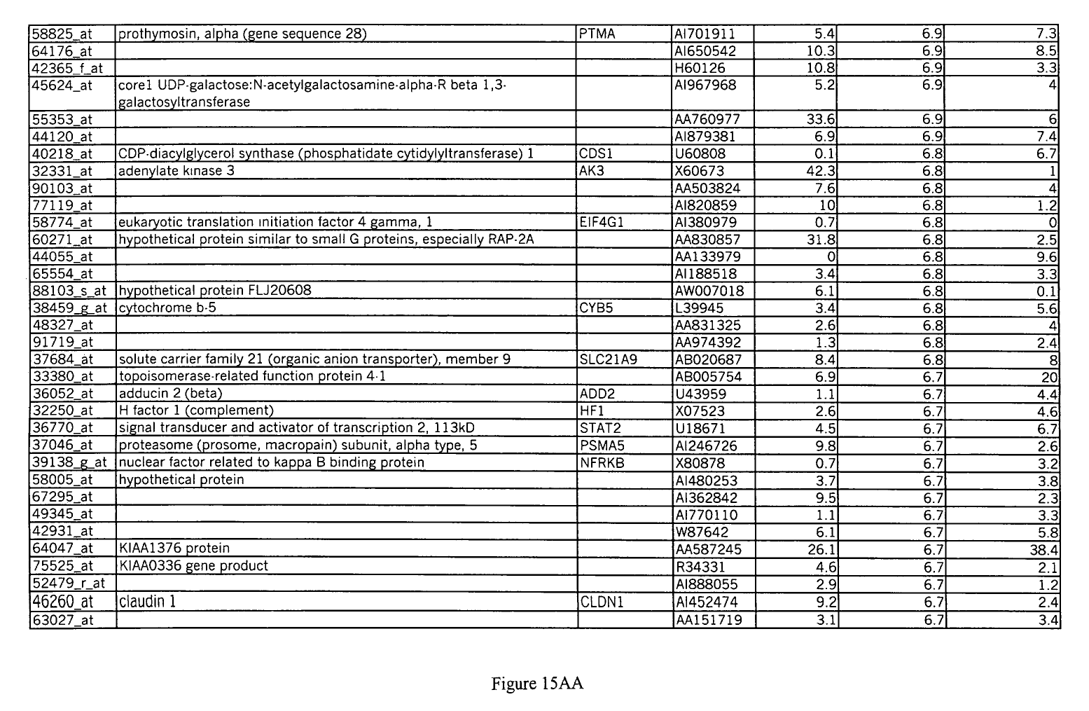

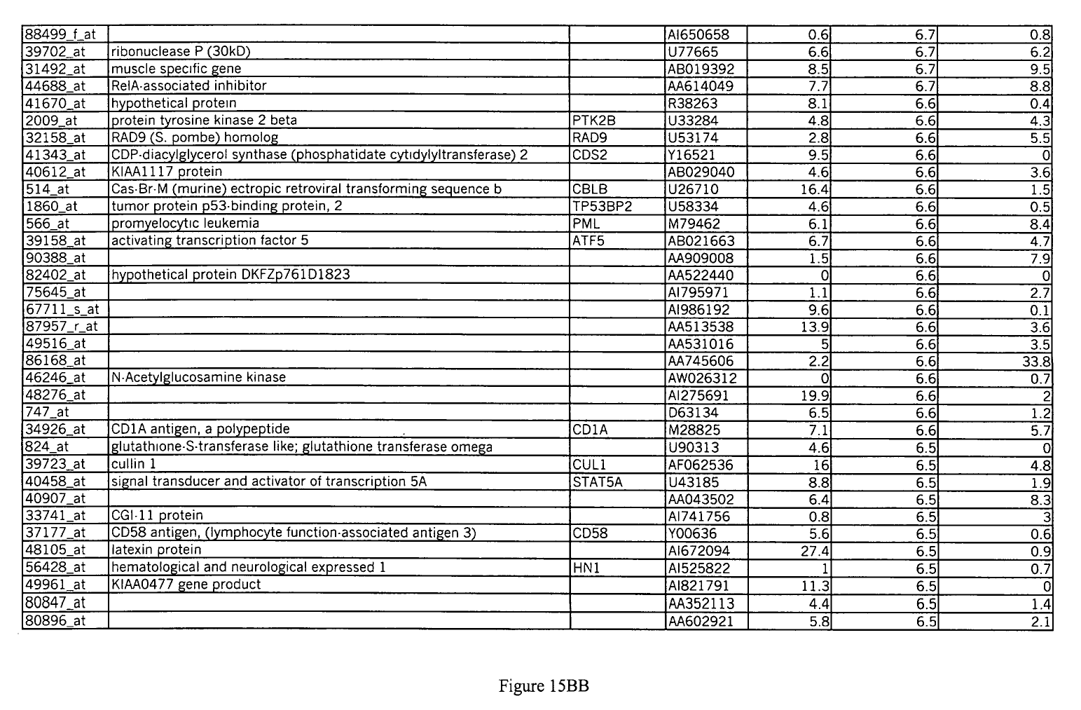

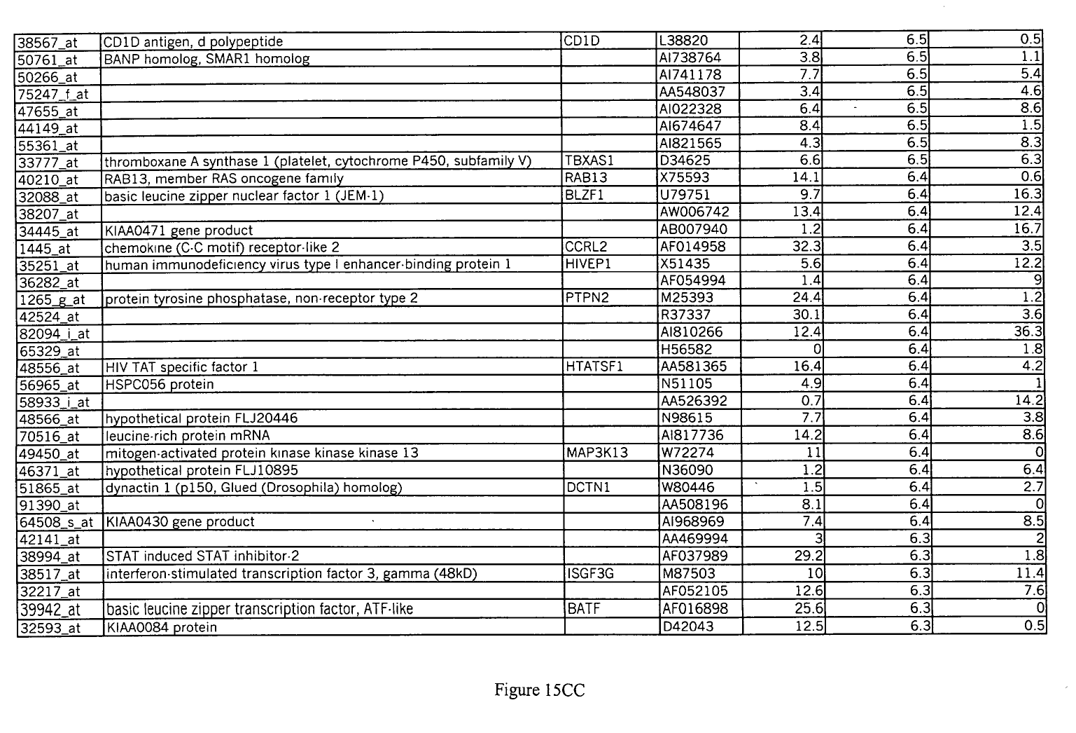

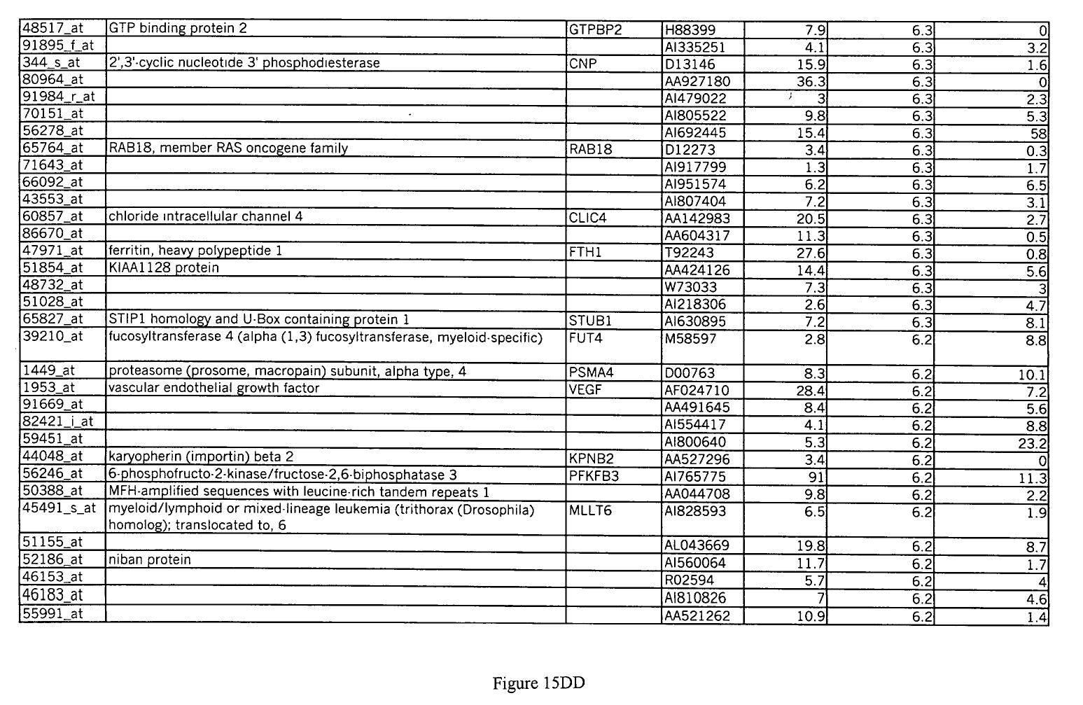

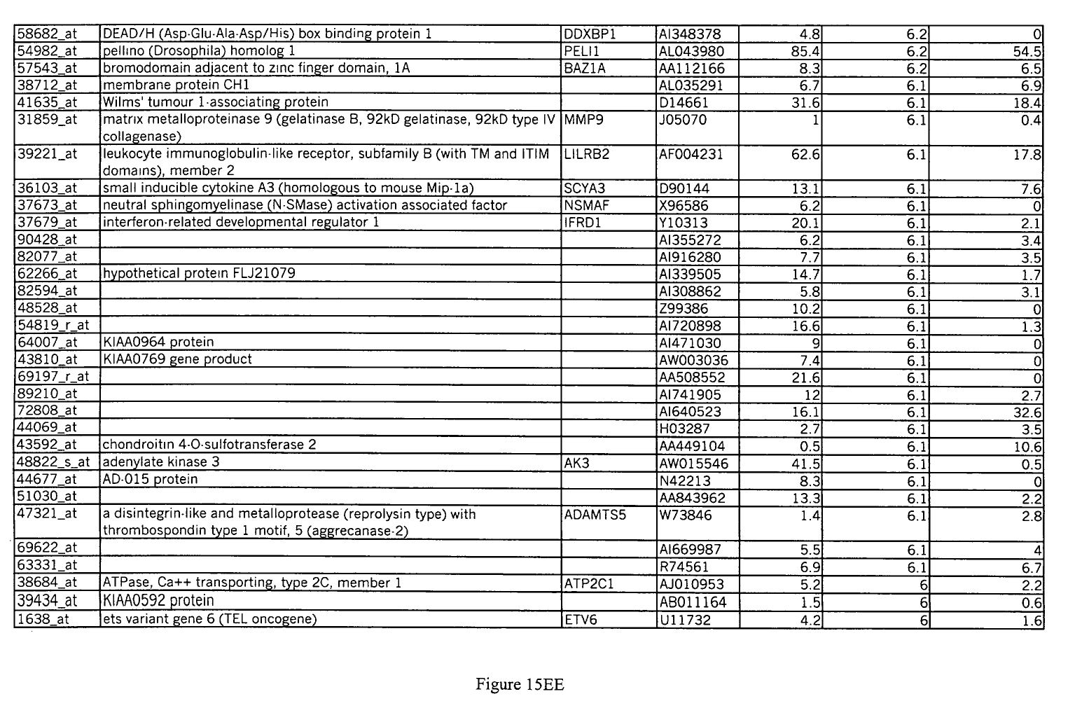

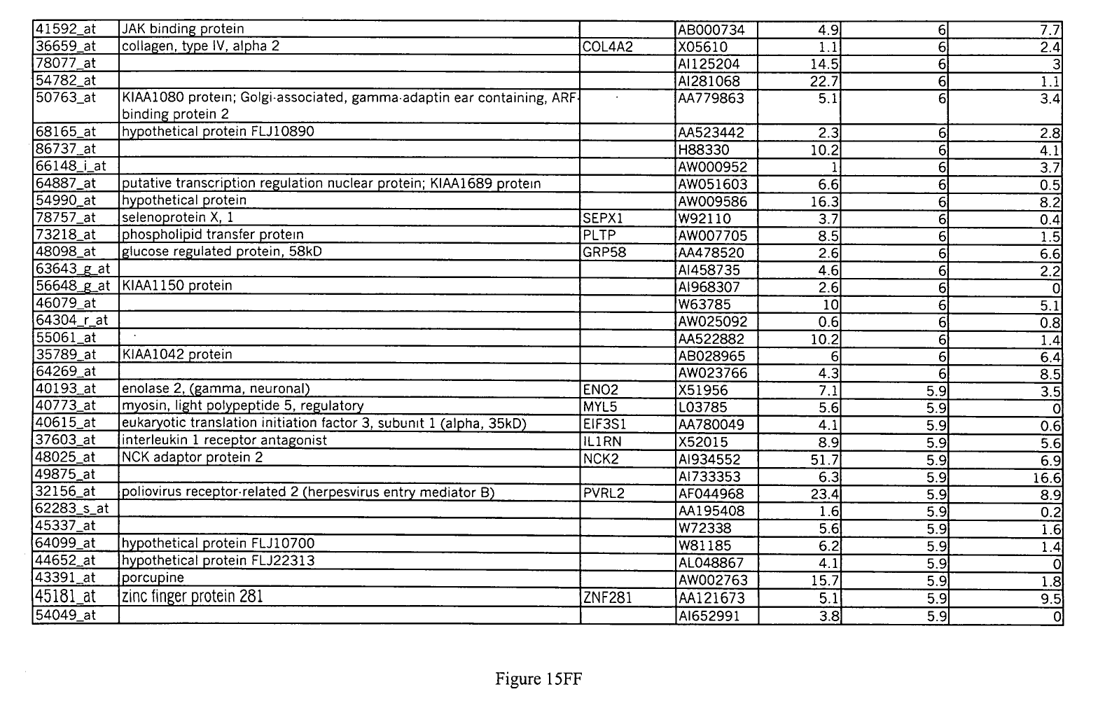

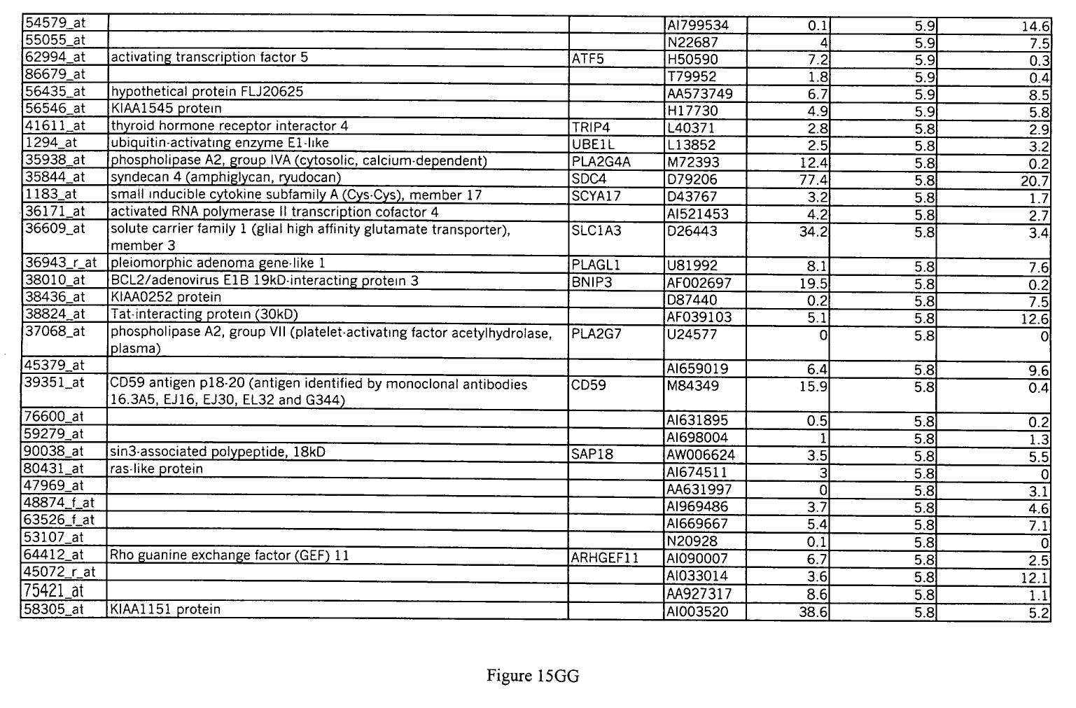

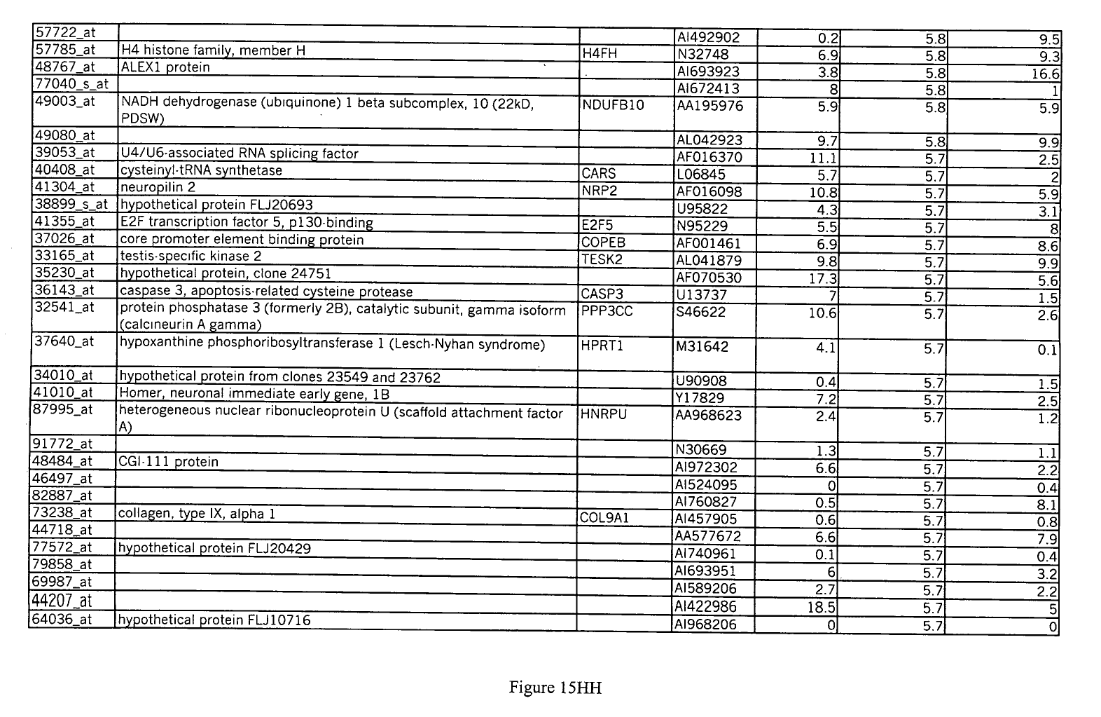

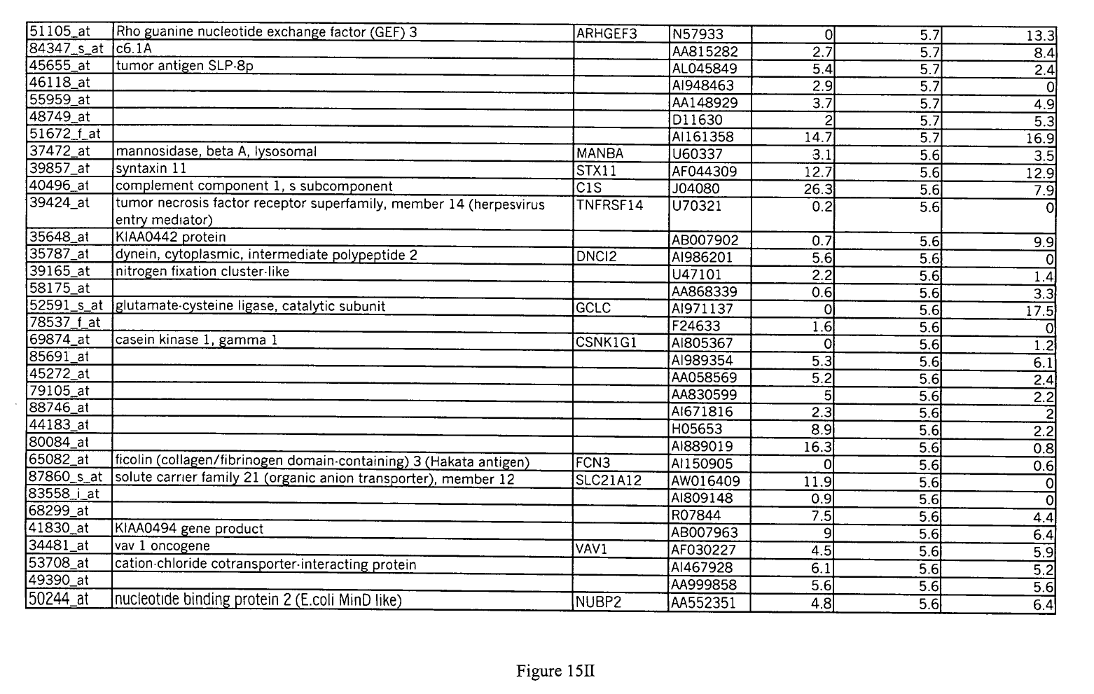

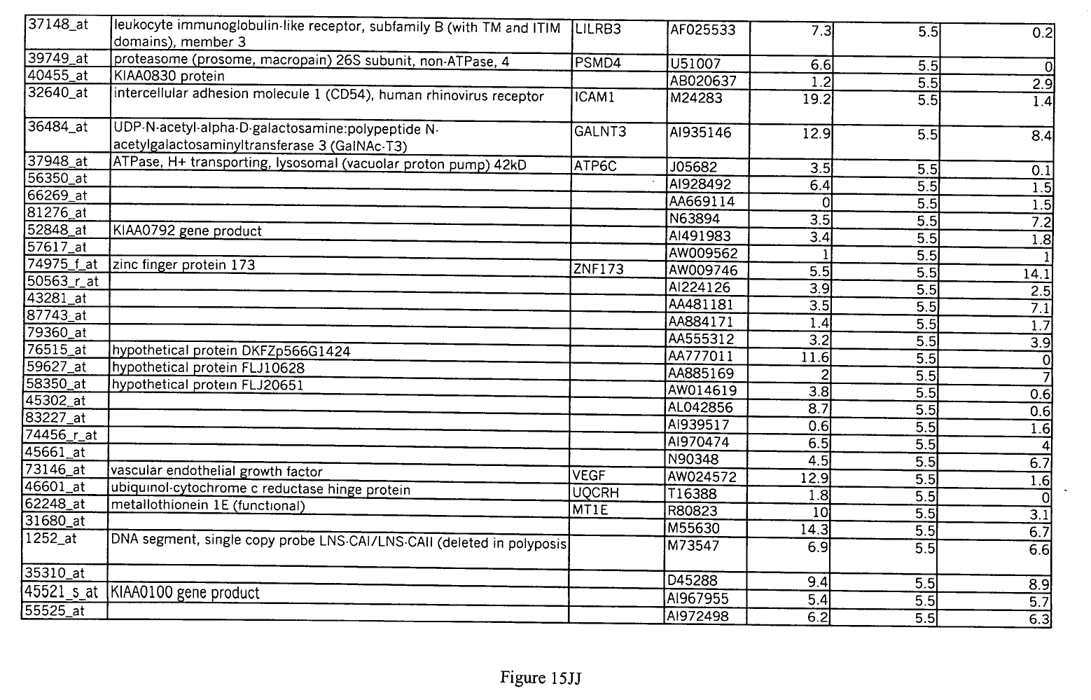

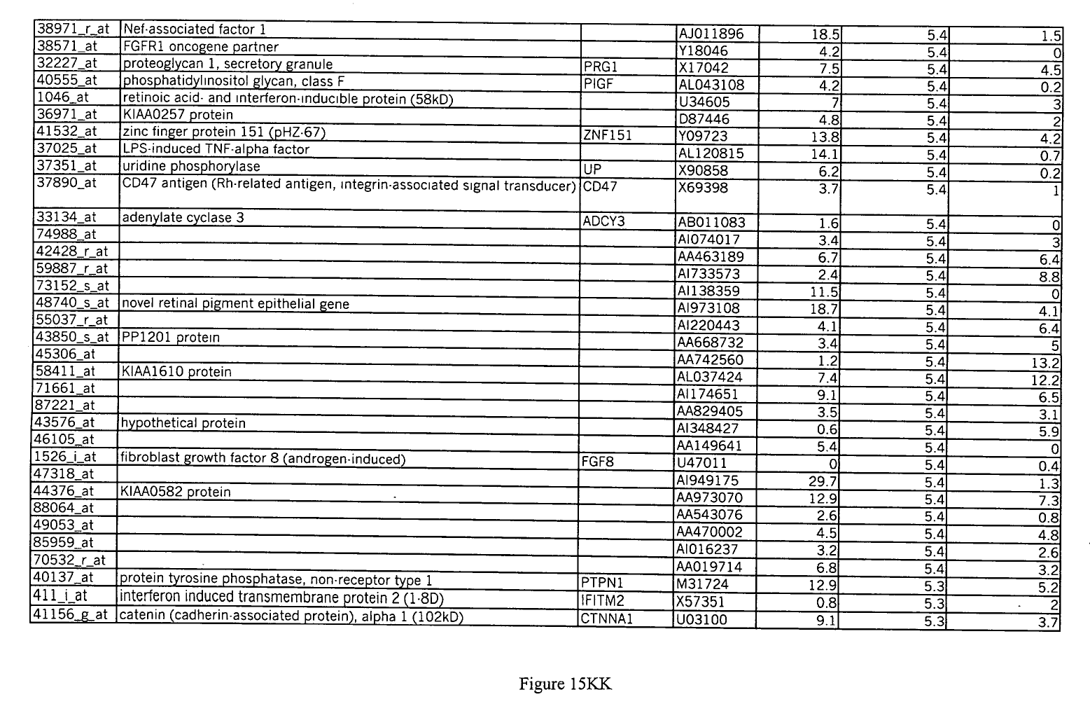

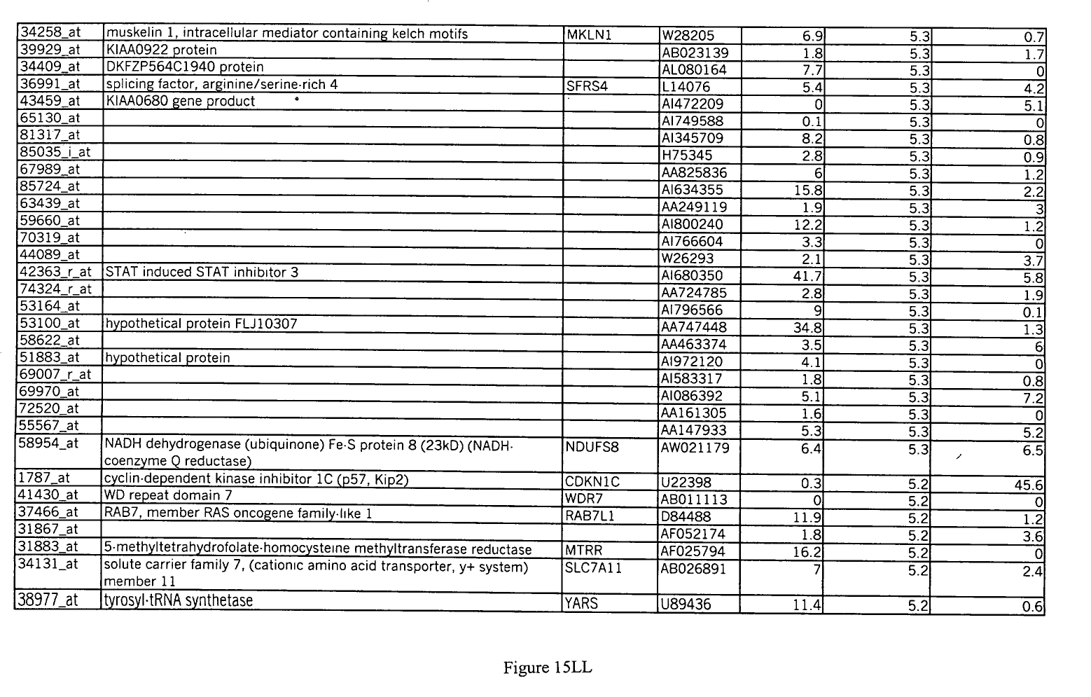

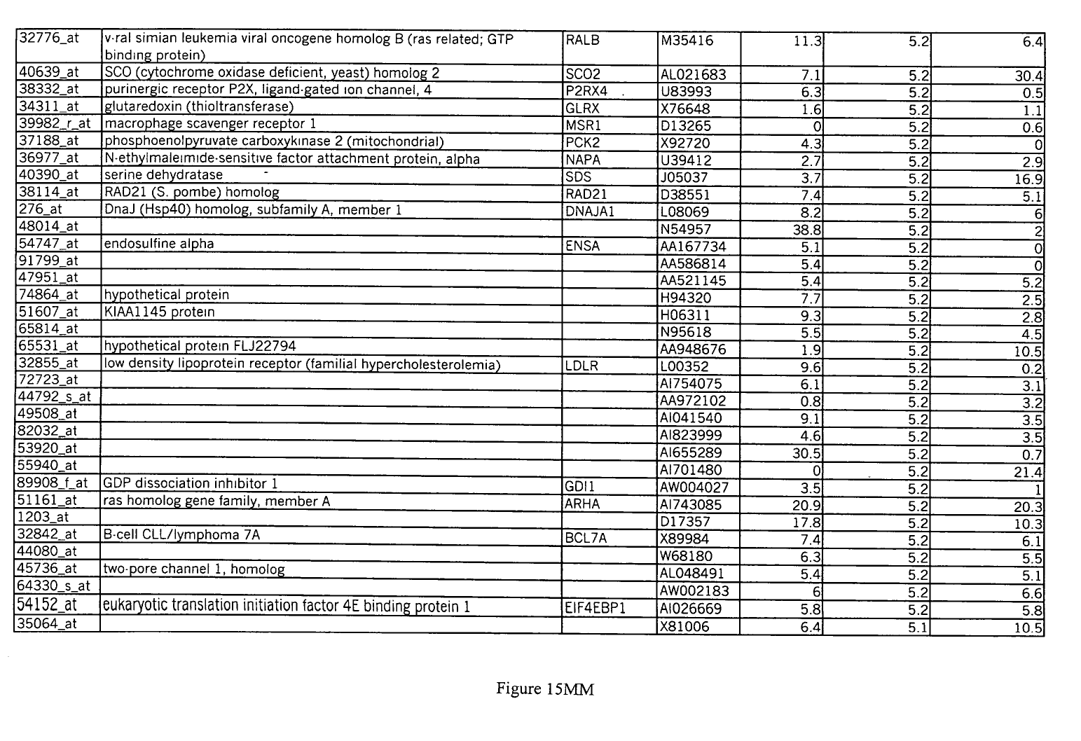

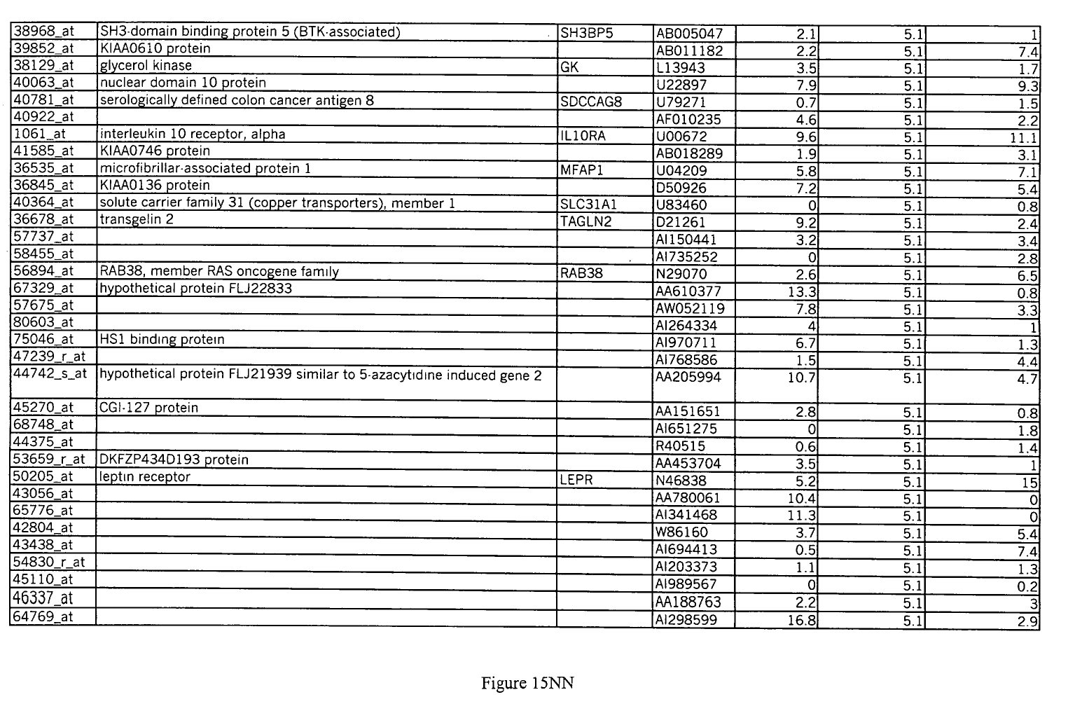

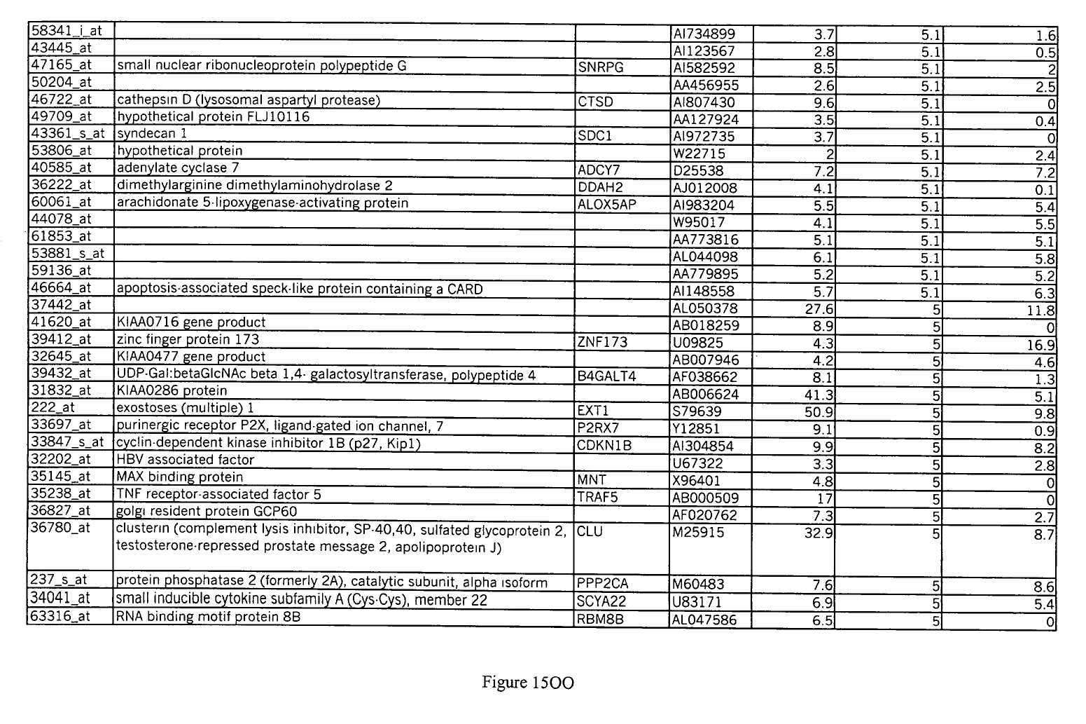

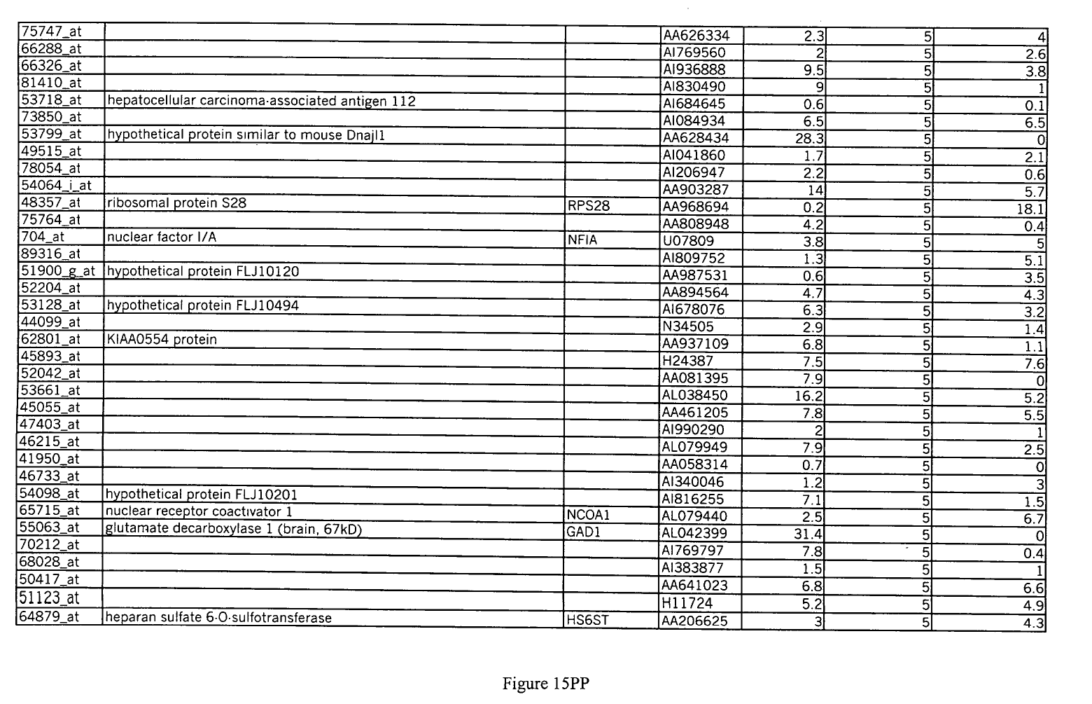

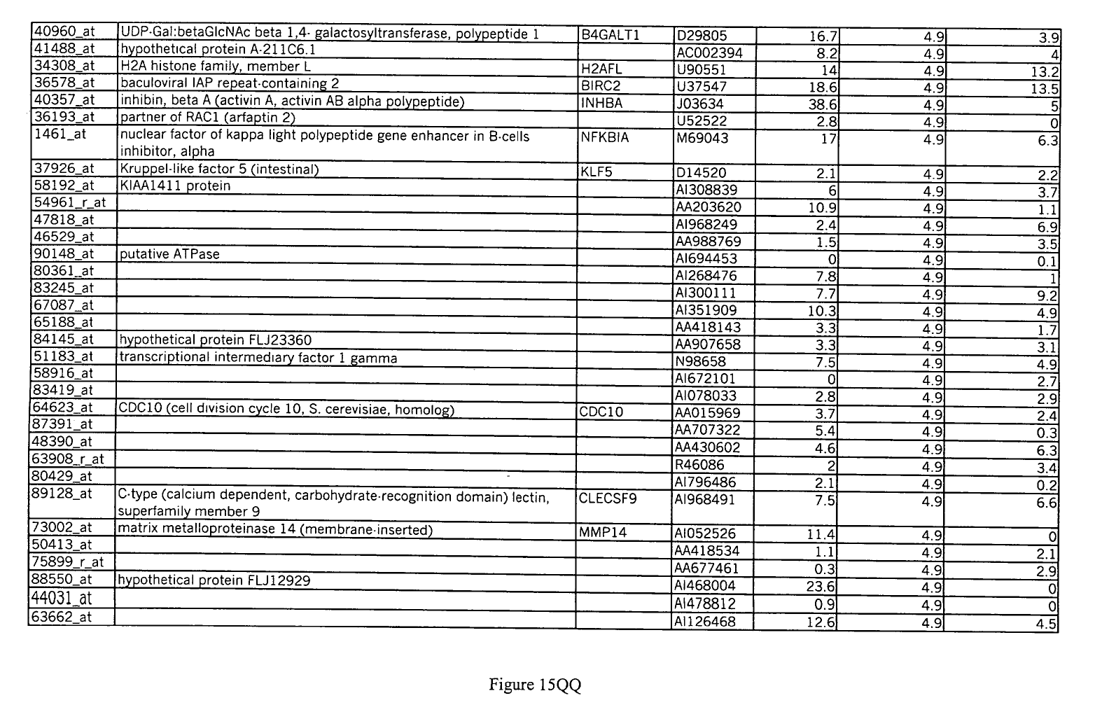

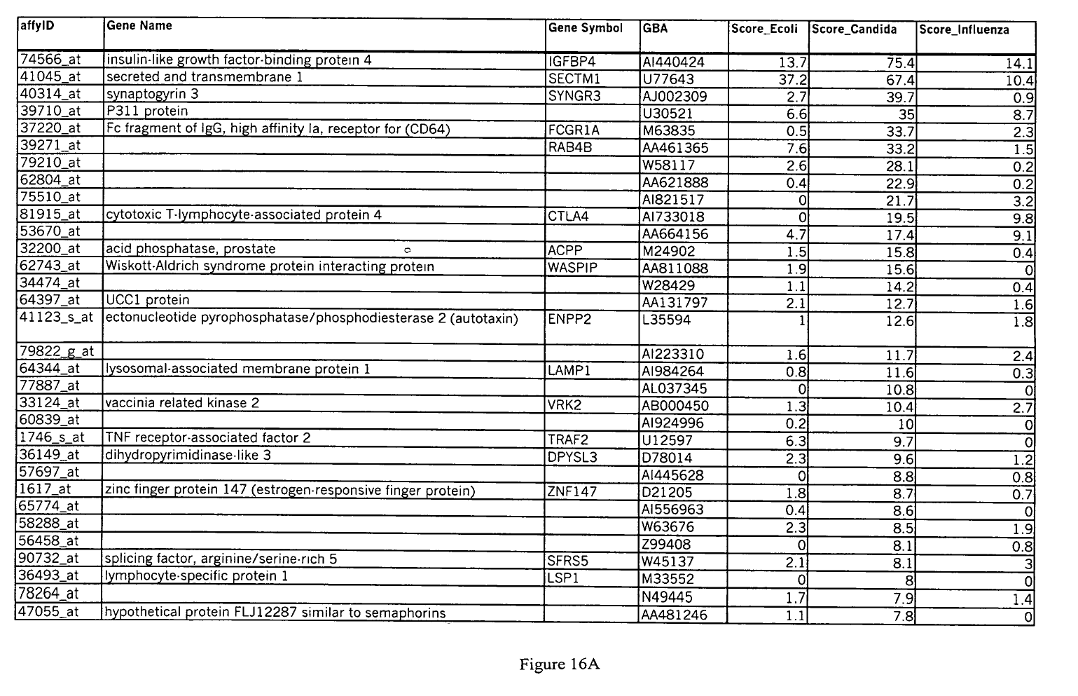

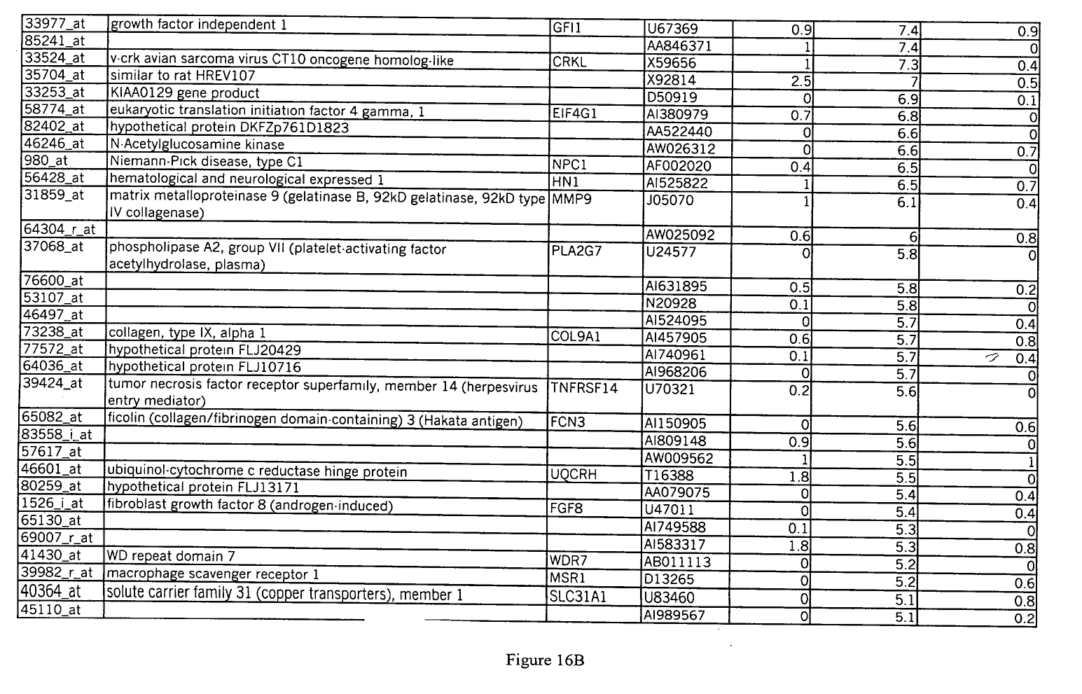

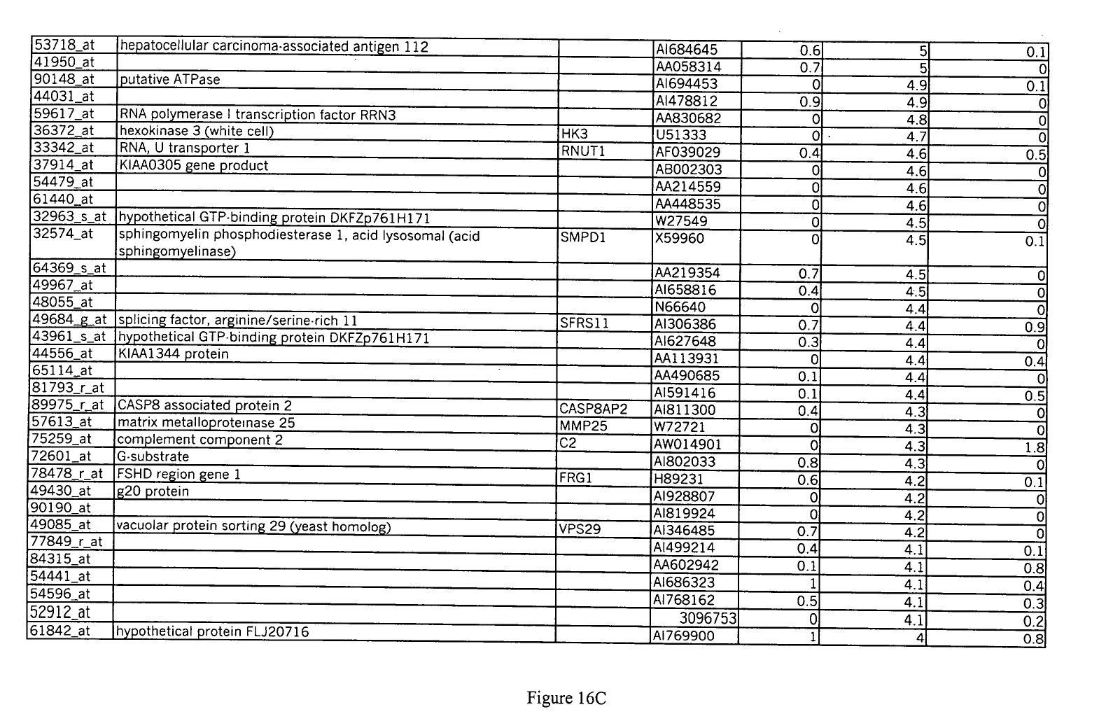

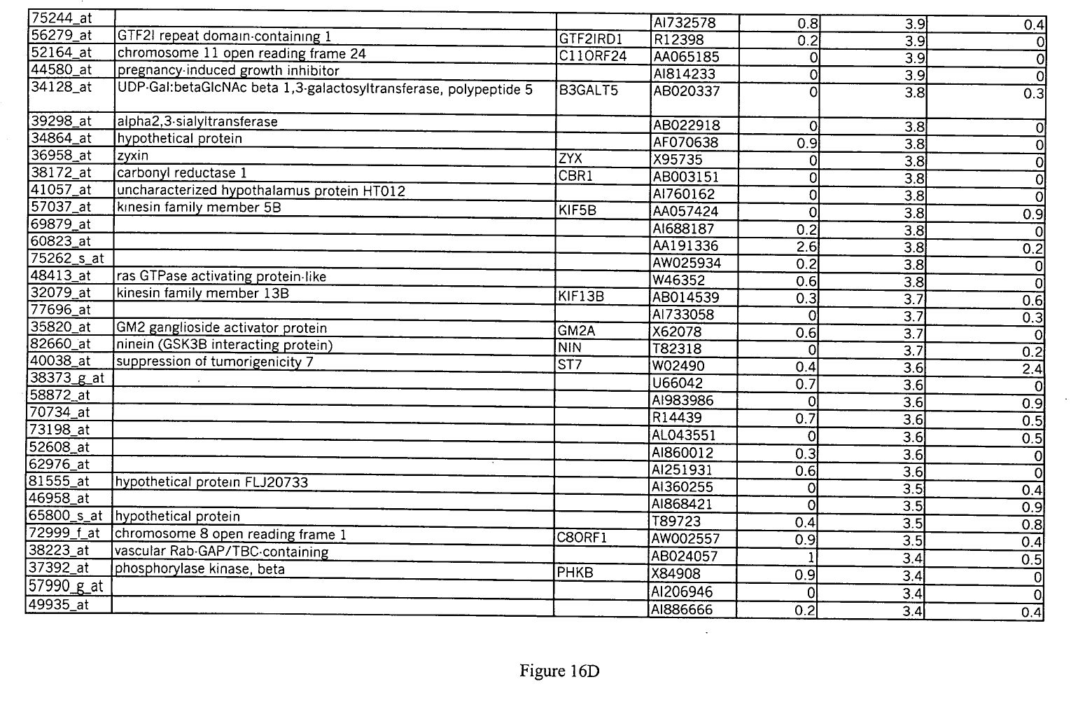

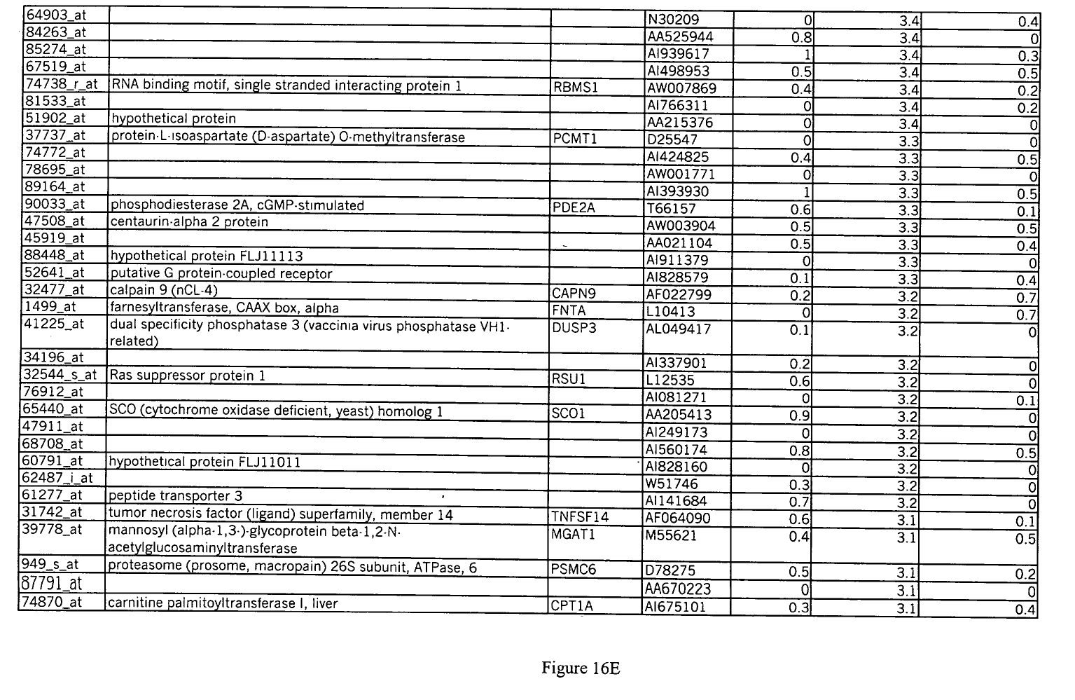

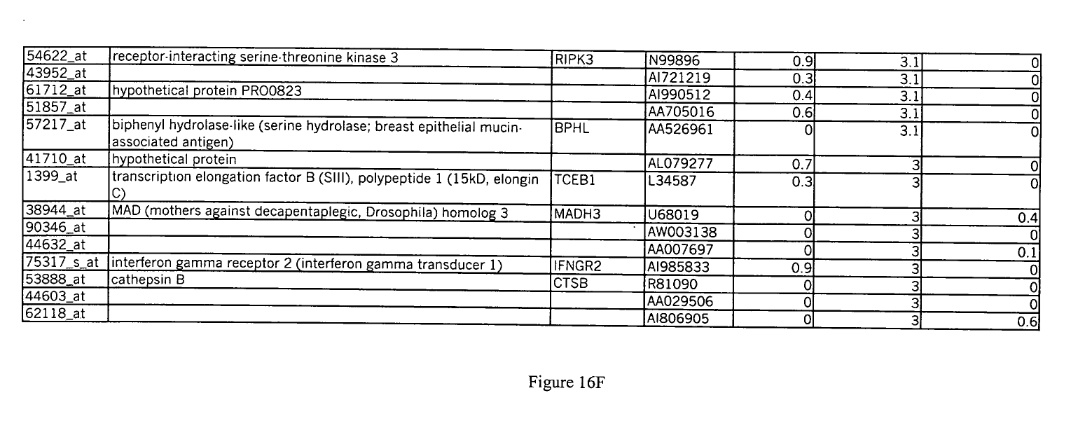

To determine if additional dendritic cell genes that are regulated in response to various pathogens could be identified, the methods described above were repeated utilizing a DNA chip (microarray) containing 60,000 genes (Affymetrix GeneChip® human genome U95 set (5 array set)). One donor was used for the results described herein. FIGS. 11A-11AAAAAAA list dendritic cell genes induced upon stimulation with E. coli. FIGS. 12A-12AA lists dendritic cell genes specifically induced by stimulation with E. coli. FIGS. 13A-13ZZZZZZ list dendritic cell genes induced upon stimulation with influenza virus. FIGS. 14A-14BB lists dendritic genes specifically induced by stimulation with influenza virus. FIGS. 15A-15KKKK list dendritic cell genes induced upon stimulation with Candida albicans. FIGS. 16A-16F list dendritic cell genes specifically induced upon stimulation with Candida albicans. FIGS. 17A-17II list dendritic genes induced by E. coli, influenza virus, and Candida albicans.

The GeneChip® human genome U95 set allowed the identification of additional genes. The expanded gene profiles described herein for the various pathogens enhance the methods of the present invention for identification of infectious agents and therapeutic targets.

What is claimed is:

1. A method of identifying a pathogen comprising the steps of:
a) isolating mRNA from one or more dendritic cells; and
b) determining gene expression of at least one stimulus-specific gene,
wherein expression of a stimulus-specific gene is indicative of infection by a pathogen to which the stimulus-specific gene is specific.

2. The method of claim 1, wherein stimulus-specific gene expression is increased.

3. The method of claim 1, wherein stimulus-specific gene expression is decreased.

4. The method of claim 1, wherein stimulus-specific gene is not expressed.

5. A method of identifying a pathogen comprising the steps of:
a) contacting immature dendritic cells with a pathogen or immunogenic components thereof;
b) isolating and labeling mRNA from said dendritic cells;
c) detecting labeled mRNA from said dendritic cells such that a gene profile is produced; and
d) analyzing the gene profile relative to one or more reference gene profile(s) such that at least one stimulus-specific gene is identified thereby identifying a pathogen for which the stimulus-specific gene is specific.

6. A method of diagnosing infection in a mammal comprising the steps of:
b) isolating mRNA from one or more dendritic cells in a mammal;
b) contacting said mRNA with at least one stimulus-responsive gene probe wherein hybridization of a stimulus-responsive probe to said mRNA is indicative of infection in said mammal.

7. The method of claim 6, wherein the stimulus-responsive probe is stimulus-specific probe.

8. The method of claim 6, wherein the stimulus-responsive probe is a common-stimulus-responsive probe.

9. A method of diagnosing infection by a pathogen in a mammal comprising the steps of;
a) isolating mRNA from one or more dendritic cells in a mammal;
b) determining gene expression of at least one-stimulus-specific gene wherein expression of a stimulus-specific gene is indicative of infection by a pathogen to which the stimulus-specific gene is specific.

10. The method of claim 9, wherein stimulus-specific gene expression is increased.

11. The method of claim 9, wherein stimulus-specific gene expression is decreased.

12. A method for predicting prognosis for an infected individual comprising the steps of:
a) analyzing gene profiles of stimulus-responsive genes, wherein a gene profile is correlated with a clinical prognosis.

13. The method of claim 12, wherein the stimulus-responsive genes are stimulus-specific.

14. The method of claim 12, wherein the stimulus-responsive genes are common stimulus-responsive genes.

15. A method of formulating a therapeutic regimen comprising the steps of:
a) identifying the pathogen; and
b) formulating the therapeutic regimen accordingly.

16. The method of claim 15, comprising a further step of repeated assessment of patient for a pathogen and formulating a therapeutic regimen.

17. A method of optimizing a vaccine comprising the steps of:
a) contacting one or more immature dendritic cells with test vaccines;
b) isolating mRNA from said dendritic cells;
c) determining gene profiles in said dendritic cells; and
d) selecting a test vaccine which elicits a gene profile indicative of an optimized vaccine.

18. An ex vivo therapeutic treatment for a pathogen comprising the steps of:
a) contacting a patient's dendritic cells with a pathogen or components thereof such that said dendritic cells become activated;
b) returning activated dendritic cells to the patient such that activated dendritic cells trigger an immune response against said pathogen.

19. An ex vivo therapeutic treatment for a tumor comprising the steps of:
a) contacting a patient's dendritic cells with tumor cells or components thereof such that said dendritic cells become activated;
b) returning activated dendritic cells to the patient such that activated dendritic cells trigger an immune response against said tumor cells or components thereof.

20. An ex vivo therapeutic treatment for autoimmunity comprising the steps of:
a) contacting a patient's dendritic cells with self-antigens or components thereof such that said dendritic cells become activated;
c) returning activated dendritic cells to the patient such that activated dendritic cells do not trigger an immune response against said self-antigens.

21. An ex vivo therapeutic treatment for graft-rejection comprising the steps of:
a) contacting a patient's dendritic cells with graft-tissue or components thereof such that said dendritic cells become activated;
b) returning activated dendritic cells to the patient such that activated dendritic cells do not trigger an immune response against graft-tissue.

22. A method of measuring the immune response to a stimulus comprising the steps of:
a) contacting one or more dendritic cells with a stimulus;
b) isolating mRNA from said dendritic cells; and
c) determining a gene profile such that at least one stimulus-responsive gene is identified which is indicative of an immune response.

23. A method of measuring the immune response to a stimulus comprising the steps of:
a) contacting dendritic cells with a stimulus;
b) isolating and labeling mRNA from said dendritic cells;
c) contacting a DNA microarray with labeled mRNA from said dendritic cells; and
d) measuring and analyzing the gene profile relative to control stimulus such that at least one stimulus-responsive gene is identified which is indicative of an immune response.

24. The method of claim 23, wherein dendritic cells are obtained from peripheral blood.

25. The method of claim 23, wherein the stimulus is selected from the group consisting of bacteria, fungi, viruses, or components thereof.

26. The method of claim 23, wherein the stimulus is selected from the group consisting of Escherichia coli, Staphylococcus aurens, influenza virus, Candida albicans, lipopolysaccharide (LPS), polyI:C, and yeast mannan.

27. The method of claim 23, wherein the stimulus is selected from the group consisting of physical, chemical, or electrical.

28. The method of claim 23, wherein the stimulus is selected from the group consisting of inorganic chemicals and organic chemicals.

29. The method of claim 23, wherein the stimulus comprises a combination selected from the group consisting of: inorganic chemicals and organic chemicals.

30. The method of claim 23, wherein the DNA microarray is Affymetrix HU 6800.

31. The method of claim 23, wherein the stimulus-responsive gene's expression is increased in response to the stimulus.

32. The method of claim 23, wherein the stimulus-responsive gene's expression is decreased in response to the stimulus.

33. The method of claim 23, wherein the stimulus-responsive gene is stimulus-specific.

34. A method of measuring the gene profile in dendritic cells in response to a stimulus comprising the steps of:
a) contacting immature dendritic cells with a stimulus;
b) isolating and labeling mRNA from said dendritic cells;
c) contacting a DNA microarray with labeled mRNA from said dendritic cells; and
d) measuring and analyzing the gene profile relative to control stimulus such that at least one stimulus-responsive gene is identified.

35. The method of claim 34, wherein dendritic cells are obtained from peripheral blood.

36. The method of claim 34, wherein the stimulus is selected from the group consisting of bacteria, fungi, viruses, or components thereof.

37. The method of claim 34, wherein the stimulus is selected from the group consisting of Escherichia coli, Staphylococcus aurens, influenza virus, Candida albicans, lipopolysaccharide (LPS), polyI:C, and yeast mannan.

38. The method of claim 34, wherein the stimulus is selected from the group consisting of physical, chemical, or electrical.

39. The method of claim 34, wherein the stimulus is selected from the group consisting of inorganic chemicals and organic chemicals.

40. The method of claim 34, wherein the stimulus comprises a combination selected from the group consisting of: inorganic chemicals and organic chemicals.

41. The method of claim 34, wherein the DNA microarray is Affymetrix HU 6800.

42. The method of claim 34, wherein the stimulus-responsive gene's expression is increased in response to the stimulus.

43. The method of claim 34, wherein the stimulus-responsive gene's expression is decreased in response to the stimulus.

44. The method of claim 34, wherein the stimulus-responsive gene is stimulus-specific.

45. A method for generating a database of stimulus-responsive genes comprising the steps of:
a) contacting immature dendritic cells with a stimulus;
b) isolating and labeling mRNA from said dendritic cells;
c) contacting a DNA microarray with labeled mRNA from said dendritic cells; and
d) measuring and analyzing the gene profile relative to control stimulus such that a database containing at least one stimulus-responsive gene is generated.

46. A method of generating a database of stimulus-specific genes comprising the steps of:
a) contacting immature dendritic cells with a stimulus;
b) isolating and labeling mRNA from said dendritic cells;
c) contacting a DNA microarray with labeled mRNA from said dendritic cells; and
d) measuring and analyzing the gene profile relative to control stimulus such that a database of stimulus-specific genes containing at least one stimulus-specific gene is generated.

47. A method of generating a database of common stimulus-responsive genes comprising the steps of:
a) contacting dendritic cells with a stimulus;
b) isolating and labeling mRNA from said dendritic cells;
c) contacting a DNA microarray with labeled mRNA from said dendritic cells; and
d) measuring and analyzing the gene profile relative to control stimulus such that a database of common stimulus-responsive genes containing at least one common stimulus-responsive gene is generated

48. A database of stimulus-responsive genes.

49. A database of stimulus-specific genes.

50. A database of common stimulus-responsive genes.

51. A method of identifying a pathogen comprising the steps of:
a) contacting one or more immature dendritic cells with a stimulus;
b) isolating mRNA from said dendritic cells; and
c) determining a gene profile such that at least one stimulus-specific gene is identified thereby identifying a pathogen for which the stimulus-specific gene is specific.

52. A method of identifying a pathogen comprising the steps of:
a) contacting one or more immature dendritic cells with a stimulus;
b) isolating and labeling mRNA from said dendritic cells;
c) contacting a DNA microarray with labeled mRNA from said dendritic cells; and
d) measuring and analyzing the gene profile relative to control stimulus such that at least one stimulus-specific gene is identified thereby identifying a pathogen for which the stimulus-specific gene is specific.

53. A method of diagnosing infection by a pathogen comprising the steps of:
a) isolating mRNA from dendritic cells; and
b) determining a gene profile such that at least one stimulus-specific gene is identified thereby identifying a pathogen for which the stimulus-specific gene is specific which is indicative of infection.

54. A method of diagnosing infection by a pathogen comprising the steps of:
a) isolating and labeling mRNA from dendritic cells;
b) contacting a DNA microarray with labeled mRNA from said dendritic cells; and
c) measuring and analyzing the gene profile relative to control stimulus such that at least one stimulus-specific gene is identified thereby identifying a pathogen for which the stimulus-specific gene is specific which is indicative of infection.

55. A method of diagnosing infection in a mammal comprising the steps of:
a) isolating proteins from one or more dendritic cells from said mammal;
b) contacting said proteins with at least one stimulus-specific antibody; wherein binding of a stimulus-specific antibody to said proteins are indicative of infection in said mammal.

56. A gene profile comprising E. coli-specific genes.

57. A gene profile comprising Candida albican-specific genes.

58. A gene profile comprising influenza virus-specific genes.
